(19) United States
(12) Patent Application Publication
(10) Pub. No.: US 2016/0168140 A1
(43) Pub. Date: Jun. 16, 2016

(54) HETEROCYCLIC COMPOUNDS AS ANTIBIOTIC POTENTIATORS

(71) Applicants: THE UNIVERSITY OF NORTH CAROLINA AT CHAPEL HILL, Chapel Hill, NC (US); SYNERECA PHARMACEUTICALS, Oklahoma City, OK (US)

(72) Inventors: Michael L. JONES, Chapel Hill, NC (US); John C. LILLY, Oklahoma City, OK (US); Sudha ANKALA, Oklahoma City, OK (US); Scott SINGLETON, Oklahoma City, OK (US)

(73) Assignees: SYNERECA PHARMACEUTICALS, Oklahoma City, OK (US); THE UNIVERSITY OF NORTH CAROLINA AT CHAPEL HILL, Chapel Hill, NC (US)

(21) Appl. No.: 14/965,759

(22) Filed: Dec. 10, 2015

Related U.S. Application Data
(60) Provisional application No. 62/091,418, filed on Dec. 12, 2014.
(60) Provisional application No. 62/216,768, filed on Sep. 10, 2015.

(51) Int. Cl. C07D 417/10; A61K 31/517; C07D 239/95; C07D 405/04; C07D 409/04; C07D 401/04; C07D 403/04; C07D 231/56; A61K 31/416; C07D 209/08; A61K 31/404; C07D 239/88; A61K 31/5377; C07D 401/14; A61K 45/06; C07D 239/94

(52) U.S. Cl. CPC C07D 417/10; C07D 239/94; A61K 31/517; C07D 239/95; C07D 405/04; C07D 409/04; C07D 401/04; C07D 403/04; C07D 231/56; A61K 31/416; C07D 209/08; A61K 31/404; C07D 239/88; A61K 31/5377; C07D 401/14; A61K 45/06

(57) ABSTRACT

The invention relates to heterocyclic compounds and their use as antibiotics and/or as antibiotic potentiators. The compounds may act as colistin potentiators and SOS inhibitors.

### CROSS-REFERENCE TO RELATED APPLICATIONS

This application claims the benefit of the priority dates of U.S. Provisional Application No. 62/091,418, filed on Dec. 12, 2014, and U.S. Provisional Application No. 62/216,768, filed on Sep. 10, 2015, both of which are hereby incorporated by reference in their entirety.

### BACKGROUND OF THE INVENTION

The emergence of multi-drug-resistant pathogens has become a serious problem in the chemotherapy of bacterial infectious diseases. One of the strategies that can be used to overcome this problem is to find new bacterial protein targets that provide functions essential for cell growth or replication; and to screen for agents that disrupt in some way that essential function. Another strategy is to improve the efficacy of existing antimicrobial drugs by countering bacterial mechanisms of drug resistance.

Adaptive resistance mutations may be caused by activation of the SOS DNA repair and mutagenesis pathway. The SOS response pathway is initiated by the accumulation of single-stranded DNA (ssDNA), promoting activation of RecA, inactivation of LexA repressor, and induction of SOS genes, including SOS error prone polymerases. Bactericidal antibiotics are powerful instigators of the SOS response. Bactericidal antibiotics can induce a common mechanism of cell death by stimulating the formation of lethal amounts of oxidative radicals (hydroxyl radicals), which activates RecA and the SOS response. E. coli strains lacking RecA are much more sensitive to bactericidal antibiotics or bacteriostatic antibiotics that are activators of the SOS response. Thus, RecA can contribute to increased tolerance to antibiotic treatment by enhancing repair of DNA damage that occurs either directly by antibiotic-induced DNA damage or indirectly from metabolic and oxidative stress. RecA-mediated repair can also induce a hypermutable state that can promote acquisition of antibiotic resistance. If DNA damage is not successfully repaired, then mutagenic polymerases (PolIV and PolV) are induced, causing mutagenesis to occur and enabling bacteria to develop antibiotic resistance. Bacteria can also develop antibiotic resistance by obtaining resistant genes from foreign DNA using the SOS response-mediated horizontal gene transfer pathway. Thus, inhibition of the SOS response can make bacteria more susceptible to antibiotics. Use of compounds that inhibit the SOS response in combination with antibiotics can help in using lower doses of antibiotics and in combating antibiotic resistant bacterial infections.

Colistin (also called polymyxin E) belongs to the polymyxin group of antibiotics. It was first isolated in Japan in 1949 from Bacillus polymyxa var. colistinus and became available for clinical use in 1959. Colistin was given as an intramuscular injection for the treatment of Gram-negative infections, but fell out of favor after aminoglycosides became available because of its significant side effects. It was later used as topical therapy as part of selective digestive tract decontamination and is still used in aerosolized form for patients with cystic fibrosis.

Side effects of colisitins—Nephrotoxicity and neurotoxicity are the most common adverse effects of polymyxins. One study showed that as many as 20% of patients experienced a decline in renal function after polymyxin therapy. However, the side effects can be reduced if smaller amounts of drug are administered. Accordingly, use of compounds that potentiate the activity of colistin would be beneficial in reducing the doses of colistin required to treat Gram negative infections.

The quinolones are a family of synthetic broad-spectrum antibacterial drugs. Quinolones act by interfering with the unwinding and duplicating of bacteria DNA. One class of quinolone antibiotics is the fluoroquinilone class, which have a fluorine atom attached to the central ring system, typically at the 6-position or C-7 position. The majority of quinolones in clinical use are fluoroquinolones.

Resistance to quinolones can evolve rapidly, even during a course of treatment. Numerous pathogens, including Staphylococcus aureus, enterococci, and Streptococcus pyogenes now exhibit resistance worldwide. Widespread veterinary usage of quinolones, in particular in Europe, has been implicated.

Though considered to be very important and necessary drugs required to treat severe and life-threatening bacterial infections, the associated antibiotic misuse remains unchecked, which has contributed to the problem of bacterial resistance. The overuse of antibiotics such as happens with children suffering from otitis media (ear infections) has given rise to a breed of super-bacteria that are resistant to antibiotics entirely. According, there is a pressing need of drugs that prevent the emergence of antibiotic resistance, or drugs that permit the continued use of existing antibiotics at approved, or lower than approved, doses by increasing their potency or increasing the susceptibility of bacteria to these drugs.

The present invention relates generally to the field of heterocyclic compounds and their use as antibiotic potentiators. The compounds of the present invention can increase the potency of known antibiotics such as colistin, quinolones, and fluoroquinolones. In some embodiments, the compounds of the present invention are antimicrobial.

### SUMMARY OF THE INVENTION

Disclosed herein, in one aspect, are compounds of formula Ia, formula IIa, or formula IIIa:

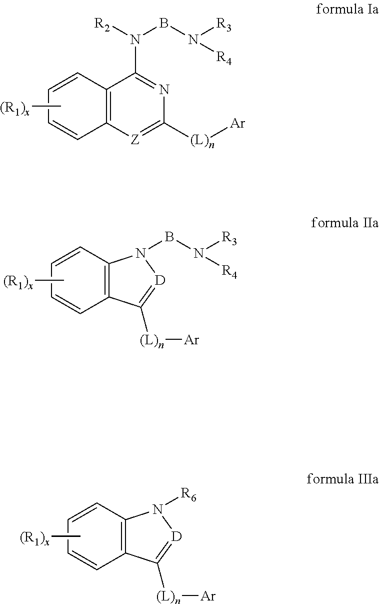

wherein,
each L is independently selected from the group consisting of C₁-C₆ alkylene, C₂-C₆ alkenylene, C₂-C₆ alkynylene, —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —NR₇C(O)—, —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-,
n is 0 or 1,
Ar is aryl, optionally substituted with one to five R₅ groups; or heteroaryl, optionally substituted with one to four R₅ groups,
B is an alkylene group, optionally interrupted by an oxygen, a carbonyl (C═O), or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,
D is CR₇ or N,
each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl, or optionally substituted heteroaryl; or two R₁ groups are linked together to form an optionally substituted 5- or 6-membered aromatic moiety or an optionally substituted 4- to 7-membered non-aromatic cyclic moiety,
R₂ is hydrogen, optionally substituted C₁-C₆ alkyl, C(O)R₇, or C₁-C₆-alkyloxycarbonyl,
R₃ and R₄ are independently hydrogen, optionally substituted C₁-C₆ alkyl, optionally substituted aryl, C(O)R₇, or C₁-C₆-alkyloxycarbonyl; or R₃ and R₄, together with the nitrogen to which they are attached, form a 4- to 6-membered heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇,
each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), optionally substituted heterocyclyl, optionally substituted C₃-C₇ cycloalkyl, or optionally substituted heteroaryl; and/or two R₅ groups are linked together to form an optionally substituted 5- or 6-membered aromatic moiety or an optionally substituted 4- to 7-membered non-aromatic cyclic moiety,
R₆ is hydrogen, optionally substituted C₁-C₆ alkyl, optionally substituted aryl-(C₁-C₆ alkylene), optionally substituted C₁-C₆-alkylcarbonyl, or C₁-C₆-alkyloxycarbonyl,
each R₇ is independently hydrogen, C₁-C₆ alkyl, or C₁-C₆ haloalkyl, and
each R₈ is independently C₁-C₆ alkyl or C₁-C₆ haloalkyl,
Z is CH or N,
x is 0, 1, 2, 3, or 4, and
if x is 0, Ar is not unsubstituted;

or a stereoisomer or a tautomer or a pharmaceutically acceptable salt thereof.

In another aspect, disclosed herein are compositions comprising a compound disclosed herein. In some embodiments, the compositions further comprise an antimicrobial agent.

In another aspect, disclosed herein are methods of treating a microbial infection in a subject in need thereof, the methods comprising administering a compound disclosed herein.

In another aspect, disclosed herein are methods of treating a microbial infection in a subject in need thereof, the methods comprising administering an antimicrobial agent and a compound disclosed herein.

In another aspect, disclosed herein are methods of increasing the activity of an antimicrobial agent, the methods comprising administering the antimicrobial agent in combination with at least one compound that potentiates the activity of the antimicrobial agent. In some embodiments, the at least one compound that potentiates the activity of the antimicrobial agent is a compound disclosed herein.

### BRIEF DESCRIPTION OF THE DRAWINGS

FIG. 1 shows time-kill assay results for colistin alone.

FIG. 2 shows time-kill assay results for colistin in combination with compound I.

FIG. 3 shows time-kill assay results for colistin in combination with compound III.

FIG. 4 shows time-kill assay results for colistin in combination with compound II.

FIG. 5 demonstrates the protective dose 50% (PD50) of colistin methanesulfonate (CMS) alone and CMS in combination with compounds MLJ-02-07A and MLJ-02-086B in a murine model of Acinetobacter baumannii clinical isolate Ab23 infection.

### DETAILED DESCRIPTION OF THE PREFERRED EMBODIMENTS

### Definitions

As used herein, the term “alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₅ alkyl). In certain embodiments, an alkyl comprises one to six carbon atoms (e.g., C₁-C₆ alkyl). In certain embodiments, an alkyl comprises one to three carbon atoms (e.g., C₁-C₃ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like. In some embodiments, an alkyl group is optionally substituted by one or more of the following substituents: halo, cyano, nitro, oxo, thioxo, —OR^{a}, —SR^{a}, —OC(O)—R^{b}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)₂R^{b}, —S(O)₂OR^{a} and —S(O)₂N(R^{a})₂ where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl and each R^{b} is independently alkyl, fluoroalkyl, carbocyclyl, carbocyclylalkyl, aryl, aralkyl, heterocyclyl, heterocyclylalkyl, heteroaryl or heteroarylalkyl.

The term “alkenyl” refers to a straight or branched hydrocarbon chain radical group consisting solely of carbon and hydrogen atoms, containing at least one double bond, and having from two to twelve carbon atoms. In certain embodiments, an alkenyl comprises two to eight carbon atoms. In other embodiments, an alkenyl comprises two to six carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. In some embodiments, an alkenyl group is optionally substituted by one or more of the following substituents: halo, cyano, nitro, oxo, thioxo, —OR^{a}, —SR^{a}, —OC(O)—R^{b}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)₂R^{b}, —S(O)₂OR^{a} and —S(O)₂N(R^{a})₂ where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl and each R^{b} is independently alkyl, fluoroalkyl, carbocyclyl, carbocyclylalkyl, aryl, aralkyl, heterocyclyl, heterocyclylalkyl, heteroaryl or heteroarylalkyl.

The term “alkynyl” refers to a straight or branched hydrocarbon chain radical group consisting solely of carbon and hydrogen atoms, containing at least one triple bond, having from two to twelve carbon atoms. In certain embodiments, an alkynyl comprises two to eight carbon atoms. In certain embodiments, an alkynyl comprises two to six carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. In some embodiments, an alkynyl group is optionally substituted by one or more of the following substituents: halo, cyano, nitro, oxo, thioxo, —OR^{a}, —SR^{a}, —OC(O)—R^{b}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)₂R^{b}, —S(O)₂OR^{a} and —S(O)₂N(R^{a})₂ where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl and each R^{b} is independently alkyl, fluoroalkyl, carbocyclyl, carbocyclylalkyl, aryl, aralkyl, heterocyclyl, heterocyclylalkyl, heteroaryl or heteroarylalkyl.

The terms “alkylene” or “alkylene chain” refer to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. In some embodiments, an alkylene chain has one to six carbons. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. In some embodiments, an alkylene chain is optionally substituted by one or more of the following substituents: halo, cyano, nitro, oxo, thioxo, —OR^{a}, —SR^{a}, —OC(O)—R^{b}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)₂R^{b}, —S(O)₂OR^{a} and —S(O)₂N(R^{a})₂ where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl and each R^{b} is independently alkyl, fluoroalkyl, carbocyclyl, carbocyclylalkyl, aryl, aralkyl, heterocyclyl, heterocyclylalkyl, heteroaryl or heteroarylalkyl.

The terms “alkenylene” or “alkenylene chain” refer to a straight or branched divalent hydrocarbon chain linking the rest of the molecule to a radical group, consisting solely of carbon and hydrogen, containing at least one double bond and having from two to twelve carbon atoms, for example, ethenylene, propenylene, n-butenylene, and the like. In some embodiments, an alkenylene chain has two to six carbons. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain. In some embodiments, an alkenylene chain is optionally substituted by one or more of the following substituents: halo, cyano, nitro, oxo, thioxo, —OR^{a}, —SR^{a}, —OC(O)—R^{b}, —N(R^{a})₂, —C(O)R^{a}, —C(O)OR^{a}, —C(O)N(R^{a})₂, —N(R^{a})C(O)OR^{a}, —N(R^{a})S(O)₂R^{b}, —S(O)₂OR^{a} and —S(O)₂N(R^{a})₂ where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl and each R^{b} is independently alkyl, fluoroalkyl, carbocyclyl, carbocyclylalkyl, aryl, aralkyl, heterocyclyl, heterocyclylalkyl, heteroaryl or heteroarylalkyl.

The terms “alkynylene” or “alkynylene chain” refer to a straight or branched divalent hydrocarbon chain linking the rest of the molecule to a radical group, consisting solely of carbon and hydrogen, containing at least one triple bond and having from two to twelve carbon atoms, for example, ethenylene, propenylene, n-butenylene, and the like. In some embodiments, an alkynylene chain has two to six carbons. The alkynylene chain is attached to the rest of the molecule through a triple bond or a single bond and to the radical group through a triple bond or a single bond. The points of attachment of the alkynylene chain to the rest of the molecule and to the radical group can be through one carbon or any two carbons within the chain. In some embodiments, an alkynylene chain is optionally substituted by one or more of the following substituents: halo, cyano, nitro, oxo, thioxo, —OR^{a}, —SR^{a}, —OC(O)—R^{b}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)₂R^{b}, —S(O)₂OR^{a} and —S(O)₂N(R^{a})₂ where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl and each R^{b} is independently alkyl, fluoroalkyl, carbocyclyl, carbocyclylalkyl, aryl, aralkyl, heterocyclyl, heterocyclylalkyl, heteroaryl or heteroarylalkyl.

The terms “aryl” or “aromatic moiety” refer to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hickel theory. In some embodiments, aryl group has 6- to 10-carbon atoms. Aryl groups include, but are not limited to, groups such as phenyl, fluorenyl, and naphthyl. In some embodiments, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{c}—OR^{a}, —R^{c}—OC(O)— R^{b}, —R^{c}—N(R^{a})₂, —R^{c}—C(O)R^{a}, —R^{c}—C(O)OR^{a}, —R^{c}—C(O)N(R^{a})₂, —R^{c}—O—R^{d}—C(O)N(R^{a})₂, —R^{c}—N(R^{a})C(O)OR^{a}, —R^{c}—N(R^{a})C(O)R^{a}, —R^{c}—N(R^{a})S(O)₂R^{b}, —R^{c}—S(O)₂OR^{a} and —R^{c}—S(O)₂N(R^{a})₂, where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently alkyl, fluoroalkyl, carbocyclyl, carbocyclylalkyl, aryl, aralkyl, heterocyclyl, heterocyclylalkyl, heteroaryl or heteroarylalkyl, each R^{c} is independently a direct bond or a straight or branched alkylene, alkenylene, or alkynylene chain, and R^{d} is a straight or branched alkylene, alkenylene, or alkynylene chain, and where each of the above substituents is unsubstituted unless otherwise indicated.

The term “aralkyl” refers to a radical of the formula —R^{e}-aryl where R^{e} is an alkylene chain as defined above, for example, benzyl, diphenylmethyl and the like. In some embodiments, the alkylene chain part of the aralkyl radical is optionally substituted as described above for an alkylene chain. In some embodiments, the aryl part of the aralkyl radical is optionally substituted as described above for an aryl group.

The term “aralkenyl” refers to a radical of the formula —R^{f}-aryl where R^{f} is an alkenylene chain as defined above. In some embodiments, the aryl part of the aralkenyl radical is optionally substituted as described above for an aryl group. In some embodiments, the alkenylene chain part of the aralkenyl radical is optionally substituted as defined above for an alkenylene group.

The term “aralkynyl” refers to a radical of the formula —R^{g}-aryl, where R^{g} is an alkynylene chain as defined above. In some embodiments, the aryl part of the aralkynyl radical is optionally substituted as described above for an aryl group. In some embodiments, the alkynylene chain part of the aralkynyl radical is optionally substituted as defined above for an alkynylene chain.

The term “carbocyclyl” refers to a stable non-aromatic monocyclic or polycyclic hydrocarbon radical consisting solely of carbon and hydrogen atoms, which may include fused or bridged ring systems, having from three to fifteen carbon atoms. In certain embodiments, a carbocyclyl comprises three to ten carbon atoms. In some embodiments, a carbocyclyl comprises five to seven carbon atoms. In some embodiments, a carbocyclyl comprises four to seven carbon atoms. In some embodiments, a carbocyclyl comprises five to six carbon atoms. The carbocyclyl is attached to the rest of the molecule by a single bond. Carbocyclyl may be saturated, (i.e., containing single C— C bonds only) or unsaturated (i.e., containing one or more double bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl,7,7-dimethyl-bicyclo[2.2.1]heptanyl, and the like. In some embodiments, the term “carbocyclyl” is meant to include carbocyclyl radicals that are optionally substituted by one or more substituents independently selected from alkyl, alkenyl, alkynyl, halo, fluoro alkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{c}—OR^{a}, —R^{c}—SR^{a}, —R^{c}—OC(O)—R^{b}, —R^{c}—N(R^{a})₂, —R^{c}—C(O)R^{a}, —R^{c}—C(O)OR^{a}, —R^{c}—C(O)N(R^{a})₂, —R^{c}—O—R^{d}—C(O)N(R^{a})₂, —R^{c}—N(R^{a})C(O)OR^{a}, —R^{c}—N(R^{a})C(O)R^{a}, —R^{c}—N(R^{a})S(O)₂R^{b}, —R^{c}—S(O)₂OR^{a} and —R^{c}—S(O)₂N(R^{a})₂ where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently alkyl, fluoroalkyl, carbocyclyl, carbocyclylalkyl, aryl, aralkyl, heterocyclyl, heterocyclylalkyl, heteroaryl or heteroarylalkyl, and each R^{c} is independently a direct bond or a straight or branched alkylene, alkenylene, or alkynylene chain.

The term “carbocyclylalkyl” refers to a radical of the formula —R^{e}-carbocyclyl where R^{e} is an alkylene chain as defined above. In some embodiments, the alkylene chain and the carbocyclyl radical are optionally substituted as defined above.

The term “fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group

The term “heterocyclyl” refers to a stable 4- to 18-membered non-aromatic ring radical that comprises three to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. In some embodiments, one or more nitrogen atoms, if present, are optionally quaternized. The heterocyclyl radical is partially or fully saturated. The heterocyclyl may be attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1, 1-dioxo-thiomorpholinyl. In some embodiments, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{c}—OR^{a}, —R^{c}—SR^{a}, —R^{c}—OC(O)—R^{b}, —R^{c}—N(R^{a})₂, —R—C(O)R^{a}, —R^{c}—C(O)OR^{a}, —R^{c}—C(O)N(R^{a})₂, —R^{c}—O—R^{d}—C(O)N(R^{a})₂, —R^{c}—N(R^{a})C(O)OR^{a}, —R^{c}—N(R^{a})C(O)R^{a}, —R^{c}—N(R^{a})S(O)₂R^{b}, —R^{c}—S(O)₂OR^{a} and —R^{c}—S(O)₂N(R^{a})₂, where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently alkyl, fluoroalkyl, carbocyclyl, carbocyclylalkyl, aryl, aralkyl, heterocyclyl, heterocyclylalkyl, heteroaryl or heteroarylalkyl, and each R^{c} is independently a direct bond or a straight or branched alkylene, alkenylene, or alkynylene chain.

The term “non-aromatic cyclic moiety” refers to a carbocyclyl or heterocyclyl radical as defined above. In some embodiments, a non-aromatic cyclic moiety is selected from a carbocyclyl radical as defined above and a heterocyclyl radical as defined above. In some embodiments, a non-aromatic cyclic moiety is a carbocyclyl radical as defined above. In some embodiments, a non-aromatic cyclic moiety is a heterocyclyl radical as defined above.

The term “heterocyclylalkyl” refers to a radical of the formula —R^{e}-heterocyclyl where R^{e} is an alkylene chain as defined above. If the heterocyclyl is a nitrogen-containing heterocyclyl, the heterocyclyl is optionally attached to the alkyl radical at the nitrogen atom. In some embodiments, the alkylene chain of the heterocyclylalkyl radical is optionally substituted as defined above for an alkylene chain. In some embodiments, the heterocyclyl part of the heterocyclylalkyl radical is optionally substituted as defined above for a heterocyclyl group.

The term “heteroaryl” refers to a radical derived from a 5- to 18-membered aromatic ring radical that comprises one to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hickel theory. Heteroaryl includes fused or bridged ring systems. In some embodiments, the heteroatom(s) in the heteroaryl radical is optionally oxidized. In some embodiments, one or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6, 7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolin yl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). In some embodiments, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{c}—OR^{a}, —R^{c}—SR^{a}, —R^{c}—OC(O)—R^{b}, —R^{c}—N(R^{a})₂, —R^{c}—C(O)R^{a}, —R^{c}—C(O)OR^{a}, —R^{c}—C(O)N(R^{a})₂, —R^{c}—O—R^{d}—C(O)N(R^{a})₂, —R^{c}—N(R^{a})C(O)OR^{a}, —R^{c}—N(R^{a})C(O)R^{a}, —R^{c}—N(R^{a})S(O)₂R^{b}, —R^{c}—S(O)₂OR^{a} and —R^{c}—S(O)₂N(R^{a})₂, where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently alkyl, fluoroalkyl, carbocyclyl, carbocyclylalkyl, aryl, aralkyl, heterocyclyl, heterocyclylalkyl, heteroaryl or heteroarylalkyl, and each R^{c} is independently a direct bond or a straight or branched alkylene, alkenylene, or alkynylene chain.

The term “aromatic moiety” refers to an aryl or heteroaryl radical as defined above. In some embodiments, an aromatic moiety is a aryl radical as defined above. In some embodiments, an aromatic moiety is a heteroaryl radical as defined above. In some embodiments, an aromatic moiety is selected from a aryl radical as defined above and a heteroaryl radical as defined above.

The term “heteroarylalkyl” refers to a radical of the formula —R^{e}-heteroaryl, where R^{e} is an alkylene chain as defined above. In some embodiments, the heteroaryl is a nitrogen-containing heteroaryl, and the heteroaryl is optionally attached to the alkyl radical at the nitrogen atom. In some embodiments, the alkylene chain of the heteroarylalkyl radical is optionally substituted as defined above for an alkylene chain. In some embodiments, the heteroaryl part of the heteroarylalkyl radical is optionally substituted as defined above for a heteroaryl group.

The term “cyano” refers to the —CN radical.

The term “oxo” refers to the ═O radical.

The term “thioxo” refers to the ═S radical.

The terms “optional” or “optionally” mean that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

The term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached. The term “epimer” refers to diastereomers that have opposite configuration at only one of two or more tetrahedral stereogenic centers present in the respective molecular entities.

The term “stereogenic center” is an atom, bearing groups such that an interchanging of any two groups leads to a stereoisomer.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

As used herein, the term “tautomers” refers to particular isomers of a compound in which a hydrogen and double bond have changed position with respect to the other atoms of the molecule. Examples of tautomers include keto-enol forms, imine-enamine forms, amide-imino alcohol forms, amidine-aminidine forms, nitroso-oxime forms, thio ketone-enethiol forms, N-nitroso-hydroxyazo forms, nitro-aci-nitro forms, and pyridione-hydroxypyridine forms.

The term “racemic” refers to a mixture of equal parts of enantiomers and which mixture is optically inactive.

The term “resolution” refers to the separation or concentration or depletion of one of the two enantiomeric forms of a molecule.

The terms “a” and “an” refer to one or more.

As used herein, the term “about” will be understood by persons of ordinary skill in the art and will vary to some extent depending upon the context in which it is used. If there are uses of the term which are not clear to a person of ordinary skill in the art, given the context in which it is used, “about” will mean up to plus or minus 10% of the particular term.

The term “treating” or “treatment” refers to administering a therapy in an amount, manner, or mode effective to improve a condition, symptom, or parameter associated with a disorder or to prevent progression of a disorder, to either a statistically significant degree or to a degree detectable to one skilled in the art. An effective amount, manner, or mode can vary depending on the subject and may be tailored to the patient.

Open terms such as “include,” “including,” “contain,” “containing” and the like mean “comprising.” The term “comprising” is not intended to exclude that in other certain embodiments, for example, an embodiment of any composition of matter, composition, method, or process, or the like, described herein, may “consist of” or “consist essentially of” the described features.

As used herein, the term “minimum inhibitory concentration” (MIC) refers to the minimum concentration of an antibiotic necessary to prevent the microorganism from growing.

As used herein, the term “protective dose 50” (PD50) refers to the dose of the antibiotic that is protective for 50% of the tested population (median dose).

### Compounds of the Invention

In one aspect, disclosed herein are compounds of formula Ia, formula IIa, or formula IIIa:

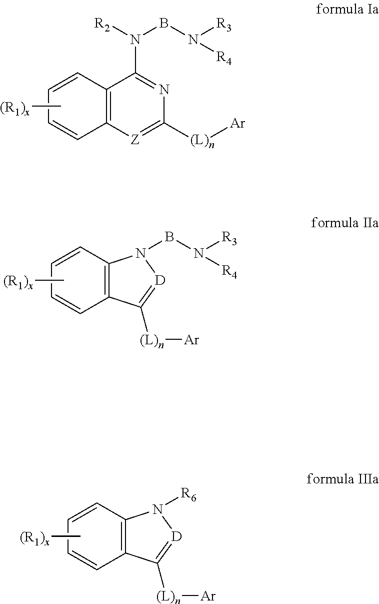

wherein,
each L is independently selected from the group consisting of C₁-C₆ alkylene, C₂-C₆ alkenylene, C₂-C₆ alkynylene, —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —NR₇C(O)—, —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-,
n is 0 or 1,
Ar is aryl, optionally substituted with one to five R₅ groups; or heteroaryl, optionally substituted with one to four R₅ groups,
B is an alkylene group, optionally interrupted by an oxygen, a carbonyl (C═O) or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,
D is CR₇ or N,
each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl, or optionally substituted heteroaryl; or two R₁ groups are linked together to form an optionally substituted 5- or 6-membered aromatic moiety or an optionally substituted 4- to 7-membered non-aromatic cyclic moiety,
R₂ is hydrogen, optionally substituted C₁-C₆ alkyl, C(O)R₇, or C₁-C₆-alkyloxycarbonyl,
R₃ and R₄ are independently hydrogen, optionally substituted C₁-C₆ alkyl, optionally substituted aryl, C(O)R₇, or C₁-C₆-alkyloxycarbonyl; or R₃ and R₄, together with the nitrogen to which they are attached, form a 4- to 6-membered heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇,
each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), optionally substituted heterocyclyl, optionally substituted C₃-C₇ cycloalkyl, or optionally substituted heteroaryl; and/or two R₅ groups are linked together to form an optionally substituted 5- or 6-membered aromatic moiety or an optionally substituted 4- to 7-membered non-aromatic cyclic moiety,
R₆ is hydrogen, optionally substituted C₁-C₆ alkyl, optionally substituted aryl-(C₁-C₆ alkylene), optionally substituted C₁-C₆-alkylcarbonyl, or C₁-C₆-alkyloxycarbonyl,
each R₇ is independently hydrogen, C₁-C₆ alkyl, or C₁-C₆ haloalkyl, and
each R₈ is independently C₁-C₆ alkyl or C₁-C₆ haloalkyl,
Z is CH or N,
x is 0, 1, 2, 3, or 4, and
if x is 0, Ar is not unsubstituted;

or a stereoisomer or a tautomer or a pharmaceutically acceptable salt thereof.

In some embodiments, the invention is a compound of formula Ia, formula IIa, or formula IIIa:

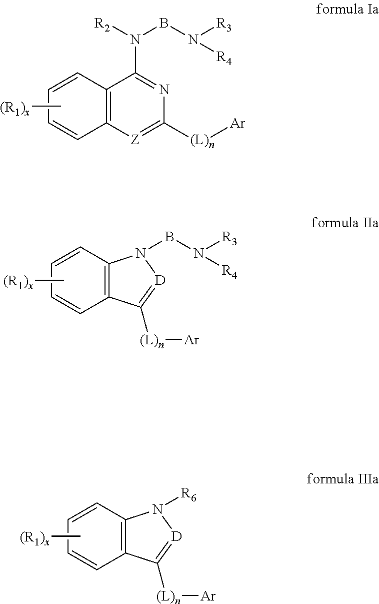

wherein,
each L is independently selected from the group consisting of C₁-C₆ alkylene, C₂-C₆ alkenylene, C₂-C₆ alkynylene, —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —NR₇C(O)—, —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-,
n is 0 or 1,
Ar is aryl, optionally substituted with one to five R₅ groups; or heteroaryl, optionally substituted with one to four R₅ groups,
B is an alkylene group, optionally interrupted by a carbonyl (C═O) or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,
D is CR₇ or N,
each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl, or optionally substituted heteroaryl; or two R₁ groups are linked together to form an optionally substituted 5- or 6-membered aromatic moiety or an optionally substituted 4- to 7-membered non-aromatic cyclic moiety,
R₂ is hydrogen, optionally substituted C₁-C₆ alkyl, C(O)R₇, or C₁-C₆-alkyloxycarbonyl,
R₃ and R₄ are independently hydrogen, optionally substituted C₁-C₆ alkyl, optionally substituted aryl, C(O)R₇, or C₁-C₆-alkyloxycarbonyl; or R₃ and R₄, together with the nitrogen to which they are attached, form a 4- to 6-membered heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇,
each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), optionally substituted heterocyclyl, or optionally substituted heteroaryl; and/or two R₅ groups are linked together to form an optionally substituted 5- or 6-membered aromatic moiety or an optionally substituted 4- to 7-membered non-aromatic cyclic moiety,
R₆ is hydrogen, optionally substituted C₁-C₆ alkyl, optionally substituted aryl-(C₁-C₆ alkylene), or C₁-C₆-alkyloxycarbonyl,
each R₇ is independently hydrogen, C₁-C₆ alkyl, or C₁-C₆ haloalkyl, and
each R₈ is independently C₁-C₆ alkyl or C₁-C₆ haloalkyl,
Z is CH or N,
x is 0, 1, 2, 3, or 4, and
if x is 0, Ar is not unsubstituted;

or a stereoisomer or a tautomer or a pharmaceutically acceptable salt thereof.

In some embodiments, the invention is a compound of formula Ia, or a stereoisomer or a tautomer or a pharmaceutically acceptable salt thereof.

In some embodiments of a compound of formula Ia, or a stereoisomer or a tautomer or a pharmaceutically acceptable salt thereof, Ar is aryl optionally substituted with one to five R₅ groups. In some embodiments of a compound of formula Ia, or a stereoisomer or a tautomer or a pharmaceutically acceptable salt thereof, Ar is phenyl or naphthyl, wherein phenyl and naphthyl are optionally substituted with one to five R₅ groups. In some embodiments of a compound of formula Ia, or a stereoisomer or a tautomer or a pharmaceutically acceptable salt thereof, Ar is phenyl substituted with one to five R₅ groups. In some embodiments of a compound of formula Ia, or a stereoisomer or a tautomer or a pharmaceutically acceptable salt thereof, Ar is phenyl substituted with one to four R₅ groups. In some embodiments of a compound of formula Ia, or a stereoisomer or a tautomer or a pharmaceutically acceptable salt thereof, Ar is phenyl substituted with one to three R₅ groups. In some embodiments of a compound of formula Ia, or a stereoisomer or a tautomer or a pharmaceutically acceptable salt thereof, Ar is phenyl substituted with one or two R₅ groups.

In some embodiments of a compound of formula Ia, or a stereoisomer or a tautomer or a pharmaceutically acceptable salt thereof, Ar is heteroaryl, optionally substituted with one to four R₅ groups. In some embodiments of a compound of formula Ia, or a stereoisomer or a tautomer or a pharmaceutically acceptable salt thereof, Ar is a 5- to 10-membered heteroaryl ring optionally substituted with one to four R₅ groups. In some embodiments of a compound of formula Ia, or a stereoisomer or a tautomer or a pharmaceutically acceptable salt thereof, Ar is a 5- to 10-membered heteroaryl ring optionally substituted with one to three R₅ groups. In some embodiments of a compound of formula Ia, or a stereoisomer or a tautomer or a pharmaceutically acceptable salt thereof, Ar is a 5- to 10-membered heteroaryl ring optionally substituted with one or two R₅ groups. In some embodiments of a compound of formula Ia, or a stereoisomer or a tautomer or a pharmaceutically acceptable salt thereof, Ar is a 5- to 10-membered heteroaryl ring optionally substituted with an R₅ group. In some embodiments of a compound of formula Ia, or a stereoisomer or a tautomer or a pharmaceutically acceptable salt thereof, Ar is indolyl, indazolyl, benzofuranyl, 1,3-benzodioxalyl or benzothiophenyl; and Ar is optionally substituted with one to four R₅ groups independently selected from halogen, C₁-C₆ alkyl, aryl-(C₁-C₆ alkylene), or C₁-C₆-alkyloxycarbonyl.

In some embodiments of a compound of formula Ia, or a stereoisomer or a tautomer or a pharmaceutically acceptable salt thereof, each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇(O)OR₈, NR₇SO₂R₈, optionally substituted aryl-(C₁-C₆ alkylene), optionally substituted aryl, optionally substituted heterocyclyl-(C₁-C₆ alkylene), optionally substituted heterocyclyl, or optionally substituted heteroaryl. In some embodiments of a compound of formula Ia, or a stereoisomer or a tautomer or a pharmaceutically acceptable salt thereof, each R₅ is independently halogen, C₁-C₆ alkyl, C₁-C₆ haloalkyl, C₁-C₆ hydroxyalkyl, C₁-C₆ alkoxy, C₁-C₆ haloalkoxy, cyano, optionally substituted aryl-(C₁-C₆ alkylene), or optionally substituted heterocyclyl-(C₁-C₆ alkylene). In some embodiments of a compound of formula Ia, or a stereoisomer or a tautomer or a pharmaceutically acceptable salt thereof, aryl is optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano; and heterocyclyl is 5- to 6-membered heterocyclyl optionally substituted with one to four groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano. In some embodiments of a compound of formula Ia, or a stereoisomer or a tautomer or a pharmaceutically acceptable salt thereof, each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, C(O)OR₇, C(O)N(R₇)₂, C(O)R₇, aryl, or aryl-(C₁-C₆ alkylene), wherein aryl and aryl-(C₁-C₆ alkylene) are optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano. In some embodiments of a compound of formula Ia, or a stereoisomer or a tautomer or a pharmaceutically acceptable salt thereof, each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, C(O)OR₇, C(O)N(R₇)₂, C(O)R₇, phenyl or benzyl, wherein phenyl and benzyl are optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano.

In some embodiments of a compound of formula Ia, or a stereoisomer or a tautomer or a pharmaceutically acceptable salt thereof, two R₅ groups are linked together to form an optionally substituted 5- or 6-membered aromatic moiety or an optionally substituted 4- to 7-membered non-aromatic cyclic moiety. In further embodiments of a compound of formula Ia, or a stereoisomer or a tautomer or a pharmaceutically acceptable salt thereof, the 5- or 6-membered aromatic moiety and the 4- to 7-membered non-aromatic cyclic moiety are optionally substituted with one or more groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), and optionally substituted heteroaryl. In still further embodiments of a compound of formula Ia, or a stereoisomer or a tautomer or a pharmaceutically acceptable salt thereof, aryl-(C₁-C₆ alkylene), aryl, heterocyclyl-(C₁-C₆ alkylene), and heteroaryl are optionally substituted with one or more groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano.

In some embodiments of a compound of formula Ia, or a stereoisomer or a tautomer or a pharmaceutically acceptable salt thereof, R₃ and R₄ are independently hydrogen, optionally substituted C₁-C₆ alkyl, optionally substituted aryl, C(O)R₇, or C₁-C₆-alkyloxycarbonyl. In some embodiments of a compound of formula Ia, or a stereoisomer or a tautomer or a pharmaceutically acceptable salt thereof, R₃ and R₄ are independently hydrogen, optionally substituted C₁-C₆ alkyl, C(O)R₇, C₁-C₆-alkyloxycarbonyl, or phenyl optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano. In some embodiments of a compound of formula Ia, or a stereoisomer or a tautomer or a pharmaceutically acceptable salt thereof, R₃ and R₄ are independently hydrogen or C₁-C₆ alkyl. In some embodiments of a compound of formula Ia, or a stereoisomer or a tautomer or a pharmaceutically acceptable salt thereof, R₃ and R₄ are independently hydrogen or C₁-C₃ alkyl. In some embodiments of a compound of formula Ia, or a stereoisomer or a tautomer or a pharmaceutically acceptable salt thereof, R₃ and R₄ are both hydrogen. In some embodiments of a compound of formula Ia, or a stereoisomer or a tautomer or a pharmaceutically acceptable salt thereof, R₃ and R₄ are both methyl. In some embodiments of a compound of formula Ia, or a stereoisomer or a tautomer or a pharmaceutically acceptable salt thereof, R₃ and R₄ are both ethyl.

In some embodiments of a compound of formula Ia, or a stereoisomer or a tautomer or a pharmaceutically acceptable salt thereof, R₃ and R₄, together with the nitrogen to which they are attached, form a 4- to 6-membered heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇. In some embodiments of a compound of formula Ia, or a stereoisomer or a tautomer or a pharmaceutically acceptable salt thereof, R₃ and R₄, together with the nitrogen to which they are attached, form a 4- to 6-membered heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇; wherein the heterocycle is azetidine, pyrrolidine, piperidine, azepane, piperazine, or homopiperazine.

In some embodiments of a compound of formula Ia, or a stereoisomer or a tautomer or a pharmaceutically acceptable salt thereof, R₂ is hydrogen or optionally substituted C₁-C₆ alkyl. In some embodiments of a compound of formula Ia, or a stereoisomer or a tautomer or a pharmaceutically acceptable salt thereof, R₂ is hydrogen or C₁-C₆ alkyl optionally substituted with halogen, N(R₇)₂, C₁-C₆ alkyl, C₁-C₆ haloalkyl, C₁-C₆ alkoxy, or C₁-C₆ haloalkoxy. In some embodiments of a compound of formula Ia, or a stereoisomer or a tautomer or a pharmaceutically acceptable salt thereof, R₂ is hydrogen or C₁-C₆ alkyl. In some embodiments of a compound of formula Ia, or a stereoisomer or a tautomer or a pharmaceutically acceptable salt thereof, R₂ is hydrogen. In some embodiments of a compound of formula Ia, or a stereoisomer or a tautomer or a pharmaceutically acceptable salt thereof, R₂ is C₁-C₆ alkyl.

In some embodiments of a compound of formula Ia, or a stereoisomer or a tautomer or a pharmaceutically acceptable salt thereof, each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇(O)OR₈, NR₇SO₂R₈, optionally substituted aryl, or optionally substituted heteroaryl. In some embodiments of a compound of formula Ia, or a stereoisomer or a tautomer or a pharmaceutically acceptable salt thereof, each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, phenyl optionally substituted with one or more groups selected from halogen, C₁-C₆ alkyl, C₁-C₆ haloalkyl, C₁-C₆ alkoxy, C₁-C₆ haloalkoxy, nitro, and cyano, or 5- to 6-membered heteroaryl optionally substituted with one or more groups selected from halogen, C₁-C₆ alkyl, C₁-C₆ haloalkyl, C₁-C₆ alkoxy, and C₁-C₆ haloalkoxy. In some embodiments of a compound of formula Ia, or a stereoisomer or a tautomer or a pharmaceutically acceptable salt thereof, each R₁ is independently halogen, C₁-C₆ alkyl, C₁-C₆ haloalkyl, C₁-C₆ alkoxy, C₁-C₆ haloalkoxy, nitro, cyano, SO₂R₈, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, phenyl optionally substituted with one or more groups selected from halogen, C₁-C₆ alkyl, C₁-C₆ haloalkyl, C₁-C₆ alkoxy, C₁-C₆ haloalkoxy, nitro, and cyano, or pyridyl optionally substituted with one or more groups selected from halogen, C₁-C₆ alkyl, C₁-C₆ haloalkyl, C₁-C₆ alkoxy, and C₁-C₆ haloalkoxy. In some embodiments of a compound of formula Ia, or a stereoisomer or a tautomer or a pharmaceutically acceptable salt thereof, each R₁ is independently halogen, C₁-C₆ alkyl, C₁-C₆ haloalkyl, C₁-C₆ alkoxy, nitro, cyano, SO₂R₈, C(O)OR₇, N(R₇)₂, NR₇C(O)R₈, phenyl optionally substituted with one or more groups selected from halogen, C₁-C₆ alkyl, C₁-C₆ haloalkyl, or pyridyl. In some embodiments of a compound of formula Ia, or a stereoisomer or a tautomer or a pharmaceutically acceptable salt thereof, each R₁ is independently halogen, C₁-C₆ alkyl, C₁-C₆ haloalkyl, nitro, cyano, SO₂R₈, N(R₇)₂, NR₇C(O)R₈, pyridyl, or phenyl optionally substituted with one or more groups selected from halogen, C₁-C₆ alkyl, and C₁-C₆ haloalkyl. In some embodiments of a compound of formula Ia, or a stereoisomer or a tautomer or a pharmaceutically acceptable salt thereof, each R₁ is independently halogen, C₁-C₆ alkyl, C₁-C₆ haloalkyl, C₁-C₆ alkoxy, C₁-C₆ haloalkoxy, N(R₇)₂, (R₇)₂N—(C₁-C₆ alkylene)-, nitro, cyano, SO₂R₈, SO₂N(R₇)₂, C(O)N(R₇)₂, or NR₇C(O)R₈.

In some embodiments of a compound of formula Ia, or a stereoisomer or a tautomer or a pharmaceutically acceptable salt thereof, x is 0, 1, 2, or 3. In some embodiments of a compound of formula Ia, or a stereoisomer or a tautomer or a pharmaceutically acceptable salt thereof, x is 0, 1, or 2. In some embodiments of a compound of formula Ia, or a stereoisomer or a tautomer or a pharmaceutically acceptable salt thereof, x is 0 or 1. In some embodiments of a compound of formula Ia, or a stereoisomer or a tautomer or a pharmaceutically acceptable salt thereof, x is 0. In some embodiments of a compound of formula Ia, or a stereoisomer or a tautomer or a pharmaceutically acceptable salt thereof, x is 1. In some embodiments of a compound of formula Ia, or a stereoisomer or a tautomer or a pharmaceutically acceptable salt thereof, x is 2.

In some embodiments of a compound of formula Ia, or a stereoisomer or a tautomer or a pharmaceutically acceptable salt thereof, two R₁ groups are linked together to form an optionally substituted 5- or 6-membered aromatic moiety or an optionally substituted 4- to 7-membered non-aromatic cyclic moiety. In further embodiments of a compound of formula Ia, or a stereoisomer or a tautomer or a pharmaceutically acceptable salt thereof, the 5- or 6-membered aromatic moiety and the 4- to 7-membered non-aromatic cyclic moiety are optionally substituted with one or more groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), and optionally substituted heteroaryl. In still further embodiments of a compound of formula Ia, or a stereoisomer or a tautomer or a pharmaceutically acceptable salt thereof, aryl-(C₁-C₆ alkylene), aryl, heterocyclyl-(C₁-C₆ alkylene), and heteroaryl are optionally substituted with one or more groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano.

In some embodiments of a compound of formula Ia, or a stereoisomer or a tautomer or a pharmaceutically acceptable salt thereof, each L is independently selected from the group consisting of C₁-C₆ alkylene, C₂-C₆ alkenylene, C₂-C₆ alkynylene, —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-. In some embodiments of a compound of formula Ia, or a stereoisomer or a tautomer or a pharmaceutically acceptable salt thereof, each L is independently selected from the group consisting of C₁-C₆ alkylene and C₂-C₆ alkenylene. In some embodiments of a compound of formula Ia, or a stereoisomer or a tautomer or a pharmaceutically acceptable salt thereof, L is C₁-C₆ alkylene. In some embodiments of a compound of formula Ia, or a stereoisomer or a tautomer or a pharmaceutically acceptable salt thereof, L is —CH₂—CH₂—. In some embodiments of a compound of formula Ia, or a stereoisomer or a tautomer or a pharmaceutically acceptable salt thereof, L is C₂-C₆ alkenylene. In some embodiments of a compound of formula Ia, or a stereoisomer or a tautomer or a pharmaceutically acceptable salt thereof, L is independently trans-CH═CH— or cis-CH═CH—. In some embodiments of a compound of formula Ia, or a stereoisomer or a tautomer or a pharmaceutically acceptable salt thereof, L is trans-CH═CH—. In some embodiments of a compound of formula Ia, or a stereoisomer or a tautomer or a pharmaceutically acceptable salt thereof, L is cis-CH═CH—. In some embodiments of a compound of formula Ia, or a stereoisomer or a tautomer or a pharmaceutically acceptable salt thereof, each L is C₂-C₆ alkynylene. In some embodiments of a compound of formula Ia, or a stereoisomer or a tautomer or a pharmaceutically acceptable salt thereof, L is independently selected from the group consisting of —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-. In some embodiments of a compound of formula Ia, or a stereoisomer or a tautomer or a pharmaceutically acceptable salt thereof, L is independently selected from the group consisting of —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), and —(C₁-C₆ alkylene)-NR₇—(C₁-C₆ alkylene). In some embodiments of a compound of formula Ia, or a stereoisomer or a tautomer or a pharmaceutically acceptable salt thereof, L is independently selected from the group consisting of —NR₇C(O)—, —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-. In some embodiments of a compound of formula Ia, or a stereoisomer or a tautomer or a pharmaceutically acceptable salt thereof, L is —NR₇C(O)—. In some embodiments of a compound of formula Ia, or a stereoisomer or a tautomer or a pharmaceutically acceptable salt thereof, L is —C(O)—. In some embodiments of a compound of formula Ia, or a stereoisomer or a tautomer or a pharmaceutically acceptable salt thereof, L is —C(O)C(O)NR₇—. In some embodiments of a compound of formula Ia, or a stereoisomer or a tautomer or a pharmaceutically acceptable salt thereof, L —C(O)NR₇—. In some embodiments of a compound of formula Ia, or a stereoisomer or a tautomer or a pharmaceutically acceptable salt thereof, L is —C(O)NR₇-(alkylene)-. In some embodiments of a compound of formula Ia, or a stereoisomer or a tautomer or a pharmaceutically acceptable salt thereof, L is —C(O)—(C₁-C₆ alkylene)-. In some embodiments of a compound of formula Ia, or a stereoisomer or a tautomer or a pharmaceutically acceptable salt thereof, L is —(C₁-C₆ alkylene)-C(O)—. In some embodiments of a compound of formula Ia, or a stereoisomer or a tautomer or a pharmaceutically acceptable salt thereof, L is —(C₁-C₆ alkylene)-C(O)—(C₁-C₆ alkylene)-.

In some embodiments of a compound of formula Ia, or a stereoisomer or a tautomer or a pharmaceutically acceptable salt thereof, n is 0 or 1. In some embodiments of a compound of formula Ia, or a stereoisomer or a tautomer or a pharmaceutically acceptable salt thereof, n is 0. In some embodiments of a compound of formula Ia, or a stereoisomer or a tautomer or a pharmaceutically acceptable salt thereof, n is 1.

In some embodiments of a compound of formula Ia, or a stereoisomer or a tautomer or a pharmaceutically acceptable salt thereof, B is alkylene interrupted by a carbonyl (C═O) or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy. In some embodiments of a compound of formula Ia, or a stereoisomer or a tautomer or a pharmaceutically acceptable salt thereof, B is alkylene interrupted by a carbonyl (C═O) and optionally substituted by one to four groups, which are independently C₁-C₆ alkyl, C₁-C₆ haloalkyl, C₁-C₆ alkoxy, or C₁-C₆ haloalkoxy. In some embodiments of a compound of formula Ia, or a stereoisomer or a tautomer or a pharmaceutically acceptable salt thereof, B is alkylene interrupted by a sulfonyl group (SO₂), and optionally substituted by one to four groups, which are independently C₁-C₆ alkyl, C₁-C₆ haloalkyl, C₁-C₆ alkoxy, or C₁-C₆ haloalkoxy. In some embodiments of a compound of formula Ia, or a stereoisomer or a tautomer or a pharmaceutically acceptable salt thereof, B is alkylene optionally interrupted by a carbonyl (C═O) or sulfonyl group (SO₂). In some embodiments of a compound of formula Ia, or a stereoisomer or a tautomer or a pharmaceutically acceptable salt thereof, B is alkylene interrupted by a carbonyl (C═O). In some embodiments of a compound of formula Ia, or a stereoisomer or a tautomer or a pharmaceutically acceptable salt thereof, B is alkylene interrupted by a sulfonyl group (SO₂). In some embodiments of a compound of formula Ia, or a stereoisomer or a tautomer or a pharmaceutically acceptable salt thereof, B is alkylene. In some embodiments of a compound of formula Ia, or a stereoisomer or a tautomer or a pharmaceutically acceptable salt thereof, B is —COCH₂—, COCH(CH₃)—, —(CH₃)₂—, —(CH₃)₃—, or —CH(CH₃)(CH₂)₃—. In some embodiments of a compound of formula Ia, or a stereoisomer or a tautomer or a pharmaceutically acceptable salt thereof, B is —(CH₃)₂—, —(CH₃)₃—, or —CH(CH₃)(CH₂)₃—.

In some embodiments of a compound of formula Ia, or a stereoisomer or a tautomer or a pharmaceutically acceptable salt thereof, Z is N. In some embodiments of a compound of formula Ia, or a stereoisomer or a tautomer or a pharmaceutically acceptable salt thereof, Z is CH.

In some embodiments of a compound of formula Ia, or a stereoisomer or a tautomer or a pharmaceutically acceptable salt thereof, Z is N; n is 1; x is 0, 1, or 2; L is trans-CH═CH—; B is an alkylene group; Ar is phenyl substituted with one to four R₅ groups. In some embodiments of a compound of formula Ia, or a stereoisomer or a tautomer or a pharmaceutically acceptable salt thereof, Z is N; n is 1; x is 0, 1, or 2; L is trans-CH═CH—; B is an alkylene group; Ar is phenyl substituted with one to four R₅ groups; R₂ is hydrogen or C₁-C₆ alkyl; and R₃ and R₄ are independently hydrogen or C₁-C₃ alkyl. In some embodiments of a compound of formula Ia, or a stereoisomer or a tautomer or a pharmaceutically acceptable salt thereof, Z is N; n is 1; x is 0, 1, or 2; L is trans-CH═CH—; B is an alkylene group; Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, C(O)OR₇, C(O)N(R₇)₂, C(O)R₇, aryl or aryl-(C₁-C₆ alkylene), wherein aryl and aryl-(C₁-C₆ alkylene) are optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano; and each R₁ is independently halogen, C₁-C₆ alkyl, C₁-C₆ haloalkyl, nitro, cyano, SO₂R₈, N(R₇)₂, NR₇C(O)R₈, pyridyl, or phenyl optionally substituted with one or more groups selected from halogen, C₁-C₆ alkyl, and C₁-C₆ haloalkyl.

In some embodiments of a compound of formula Ia, or a stereoisomer or a tautomer or a pharmaceutically acceptable salt thereof, Z is N; n is 1; x is 0, 1, or 2; L is C₁-C₆ alkylene; B is an alkylene group; Ar is phenyl substituted with one to four R₅ groups. In some embodiments of a compound of formula Ia, or a stereoisomer or a tautomer or a pharmaceutically acceptable salt thereof, Z is N; n is 1; x is 0, 1, or 2; L is C₁-C₆ alkylene; B is an alkylene group; Ar is phenyl substituted with one to four R₅ groups; R₂ is hydrogen or C₁-C₆ alkyl; and R₃ and R₄ are independently hydrogen or C₁-C₃ alkyl. In some embodiments of a compound of formula Ia, or a stereoisomer or a tautomer or a pharmaceutically acceptable salt thereof, Z is N; n is 1; x is 0, 1, or 2; L is C₁-C₆ alkylene; B is an alkylene group; Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, C(O)OR₇, C(O)N(R₇)₂, C(O)R₇, aryl, or aryl-(C₁-C₆ alkylene), wherein aryl and aryl-(C₁-C₆ alkylene) are optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano; and each R₁ is independently halogen, C₁-C₆ alkyl, C₁-C₆ haloalkyl, nitro, cyano, SO₂R₈, N(R₇)₂, NR₇C(O)R₈, pyridyl, or phenyl optionally substituted with one or more groups selected from halogen, C₁-C₆ alkyl, and C₁-C₆ haloalkyl. In some embodiments of a compound of formula Ia, or a stereoisomer or a tautomer or a pharmaceutically acceptable salt thereof, Z is N; n is 1; x is 0, 1, or 2; L is ethylene; B is an alkylene group; Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, C(O)OR₇, C(O)N(R₇)₂, C(O)R₇, aryl, or aryl-(C₁-C₆ alkylene), wherein aryl and aryl-(C₁-C₆ alkylene) are optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano; and each R₁ is independently halogen, C₁-C₆ alkyl, C₁-C₆ haloalkyl, nitro, cyano, SO₂R₈, N(R₇)₂, NR₇C(O)R₈, pyridyl, or phenyl optionally substituted with one or more groups selected from halogen, C₁-C₆ alkyl, and C₁-C₆ haloalkyl.

In some embodiments of a compound of formula Ia, or a stereoisomer or a tautomer or a pharmaceutically acceptable salt thereof, Z is N; n is 0; x is 0, 1, or 2; and B is an alkylene group.

In some embodiments of a compound of formula Ia, or a stereoisomer or a tautomer or a pharmaceutically acceptable salt thereof, Z is N; n is 0; x is 0, 1, or 2; B is an alkylene group; and Ar is phenyl substituted with one to four R₅ groups. In some embodiments of a compound of formula Ia, or a stereoisomer or a tautomer or a pharmaceutically acceptable salt thereof, Z is N; n is 0; x is 0, 1, or 2; B is an alkylene group; Ar is phenyl substituted with one to four R₅ groups; R₂ is hydrogen or C₁-C₆ alkyl; and R₃ and R₄ are independently hydrogen or C₁-C₃ alkyl. In some embodiments of a compound of formula Ia, or a stereoisomer or a tautomer or a pharmaceutically acceptable salt thereof, Z is N; n is 0; x is 0, 1, or 2; B is an alkylene group; Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, C(O)OR₇, C(O)N(R₇)₂, C(O)R₇, aryl, or aryl-(C₁-C₆ alkylene), wherein aryl and aryl-(C₁-C₆ alkylene) are optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano; and each R₁ is independently halogen, C₁-C₆ alkyl, C₁-C₆ haloalkyl, nitro, cyano, SO₂R₈, N(R₇)₂, NR₇C(O)R₈, pyridyl, or phenyl optionally substituted with one or more groups selected from halogen, C₁-C₆ alkyl, and C₁-C₆ haloalkyl.

In some embodiments of a compound of formula Ia, or a stereoisomer or a tautomer or a pharmaceutically acceptable salt thereof, Z is N; n is 0; x is 0, 1, or 2; B is an alkylene group; Ar is naphthyl. In some embodiments of a compound of formula Ia, or a stereoisomer or a tautomer or a pharmaceutically acceptable salt thereof, Z is N; n is 0; x is 0, 1, or 2; B is an alkylene group; Ar is naphthyl; R₂ is hydrogen or C₁-C₆ alkyl; and R₃ and R₄ are independently hydrogen or C₁-C₃ alkyl. In some embodiments of a compound of formula Ia, or a stereoisomer or a tautomer or a pharmaceutically acceptable salt thereof, Z is N; n is 0; x is 0, 1, or 2; B is an alkylene group; Ar is naphthyl; R₂ is hydrogen or C₁-C₆ alkyl; R₃ and R₄ are independently hydrogen or C₁-C₃ alkyl; and each R₁ is independently halogen, C₁-C₆ alkyl, C₁-C₆ haloalkyl, nitro, cyano, SO₂R₈, N(R₇)₂, NR₇C(O)R₈, pyridyl, or phenyl optionally substituted with one or more groups selected from halogen, C₁-C₆ alkyl, and C₁-C₆ haloalkyl.

In some embodiments of a compound of formula Ia, or a stereoisomer or a tautomer or a pharmaceutically acceptable salt thereof, Z is N; n is 0; x is 0, 1, or 2; B is an alkylene group; Ar is heteroaryl optionally substituted with one to four R₅ groups. In some embodiments of a compound of formula Ia, or a stereoisomer or a tautomer or a pharmaceutically acceptable salt thereof, Z is N; n is 0; x is 0, 1, or 2; B is an alkylene group; Ar is heteroaryl optionally substituted with one to four R₅ groups; R₂ is hydrogen or C₁-C₆ alkyl; and R₃ and R₄ are independently hydrogen or C₁-C₃ alkyl. In some embodiments of a compound of formula Ia, or a stereoisomer or a tautomer or a pharmaceutically acceptable salt thereof, Z is N; n is 0; x is 0, 1, or 2; B is an alkylene group; Ar is heteroaryl optionally substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, C(O)OR₇, C(O)N(R₇)₂, C(O)R₇, aryl, or aryl-(C₁-C₆ alkylene), wherein aryl and aryl-(C₁-C₆ alkylene) are optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano; and each R₁ is independently halogen, C₁-C₆ alkyl, C₁-C₆ haloalkyl, nitro, cyano, SO₂R₈, N(R₇)₂, NR₇C(O)R₈, pyridyl, or phenyl optionally substituted with one or more groups selected from halogen, C₁-C₆ alkyl, and C₁-C₆ haloalkyl. In some embodiments of a compound of formula Ia, or a stereoisomer or a tautomer or a pharmaceutically acceptable salt thereof, Z is N; n is 0; x is 0, 1, or 2; B is an alkylene group; Ar is indolyl, indazolyl, benzofuranyl, 1,3-benzodioxalyl, or benzothiophenyl; and Ar is optionally substituted with one to four R₅ groups independently selected from halogen, C₁-C₆ alkyl, aryl-(C₁-C₆ alkylene), or C₁-C₆-alkyloxycarbonyl; R₂ is hydrogen or C₁-C₆ alkyl; R₃ and R₄ are independently hydrogen or C₁-C₃ alkyl; and each R₁ is independently halogen, C₁-C₆ alkyl, C₁-C₆ haloalkyl, nitro, cyano, SO₂R₈, N(R₇)₂, NR₇C(O)R₈, pyridyl, or phenyl optionally substituted with one or more groups selected from halogen, C₁-C₆ alkyl, and C₁-C₆ haloalkyl.

In some embodiments of a compound of formula Ia, or a stereoisomer or a tautomer or a pharmaceutically acceptable salt thereof, Z is N; n is 1; x is 0, 1, or 2; L is —(C₁-C₆ alkylene)-O—; B is an alkylene group; Ar is phenyl substituted with one to four R₅ groups. In some embodiments of a compound of formula Ia, or a stereoisomer or a tautomer or a pharmaceutically acceptable salt thereof, Z is N; n is 1; x is 0, 1, or 2; L is —(C₁-C₆ alkylene)-O—; B is an alkylene group; Ar is phenyl substituted with one to four R₅ groups; R₂ is hydrogen or C₁-C₆ alkyl; and R₃ and R₄ are independently hydrogen or C₁-C₃ alkyl. In some embodiments of a compound of formula Ia, or a stereoisomer or a tautomer or a pharmaceutically acceptable salt thereof, Z is N; n is 1; x is 0, 1, or 2; L is —(C₁-C₆ alkylene)-O—; B is an alkylene group; Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, C(O)OR₇, C(O)N(R₇)₂, C(O)R₇, aryl, or aryl-(C₁-C₆ alkylene), wherein aryl and aryl-(C₁-C₆ alkylene) are optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano; and each R₁ is independently halogen, C₁-C₆ alkyl, C₁-C₆ haloalkyl, nitro, cyano, SO₂R₈, N(R₇)₂, NR₇C(O)R₈, pyridyl, or phenyl optionally substituted with one or more groups selected from halogen, C₁-C₆ alkyl, and C₁-C₆ haloalkyl.

In some embodiments of a compound of formula Ia, or a stereoisomer or a tautomer or a pharmaceutically acceptable salt thereof, Z is N; n is 1; x is 0, 1, or 2; L is —C(O)NR₇-(alkylene)-; B is an alkylene group; Ar is phenyl substituted with one to four R₅ groups. In some embodiments of a compound of formula Ia, or a stereoisomer or a tautomer or a pharmaceutically acceptable salt thereof, Z is N; n is 1; x is 0, 1, or 2; L is —C(O)NR₇-(alkylene)-; B is an alkylene group; Ar is phenyl substituted with one to four R₅ groups; R₂ is hydrogen or C₁-C₆ alkyl; and R₃ and R₄ are independently hydrogen or C₁-C₃ alkyl. In some embodiments of a compound of formula Ia, or a stereoisomer or a tautomer or a pharmaceutically acceptable salt thereof, Z is N; n is 1; x is 0, 1, or 2; L is —C(O)NR₇-(alkylene)-; B is an alkylene group; Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, C(O)OR₇, C(O)N(R₇)₂, C(O)R₇, aryl, or aryl-(C₁-C₆ alkylene), wherein aryl and aryl-(C₁-C₆ alkylene) are optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano; and each R₁ is independently halogen, C₁-C₆ alkyl, C₁-C₆ haloalkyl, nitro, cyano, SO₂R₈, N(R₇)₂, NR₇C(O)R₈, pyridyl, or phenyl optionally substituted with one or more groups selected from halogen, C₁-C₆ alkyl, and C₁-C₆ haloalkyl.

In some embodiments of a compound of formula Ia, or a stereoisomer or a tautomer or a pharmaceutically acceptable salt thereof, Z is N; n is 1; x is 0, 1, or 2; L is —NR₇C(O)—; B is an alkylene group; Ar is phenyl substituted with one to four R₅ groups. In some embodiments of a compound of formula Ia, or a stereoisomer or a tautomer or a pharmaceutically acceptable salt thereof, Z is N; n is 1; x is 0, 1, or 2; L is —NR₇C(O)—; B is an alkylene group; Ar is phenyl substituted with one to four R₅ groups; R₂ is hydrogen or C₁-C₆ alkyl; and R₃ and R₄ are independently hydrogen or C₁-C₃ alkyl. In some embodiments of a compound of formula Ia, or a stereoisomer or a tautomer or a pharmaceutically acceptable salt thereof, Z is N; n is 1; x is 0, 1, or 2; L is —NR₇C(O)—; B is an alkylene group; Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, C(O)OR₇, C(O)N(R₇)₂, C(O)R₇, aryl, or aryl-(C₁-C₆ alkylene), wherein aryl and aryl-(C₁-C₆ alkylene) are optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano; and each R₁ is independently halogen, C₁-C₆ alkyl, C₁-C₆ haloalkyl, nitro, cyano, SO₂R₈, N(R₇)₂, NR₇C(O)R₈, pyridyl, or phenyl optionally substituted with one or more groups selected from halogen, C₁-C₆ alkyl, and C₁-C₆ haloalkyl.

In some embodiments, the invention is a compound of formula IIa or a stereoisomer or a tautomer or a pharmaceutically acceptable salt thereof.

In some embodiments of a compound of formula IIa, or a stereoisomer or a tautomer or a pharmaceutically acceptable salt thereof, Ar is aryl optionally substituted with one to five R₅ groups. In some embodiments of a compound of formula IIa, or a stereoisomer or a tautomer or a pharmaceutically acceptable salt thereof, Ar is phenyl or naphthyl, wherein phenyl and naphthyl are optionally substituted with one to five R₅ groups. In some embodiments of a compound of formula IIa, or a stereoisomer or a tautomer or a pharmaceutically acceptable salt thereof, Ar is phenyl substituted with one to five R₅ groups. In some embodiments of a compound of formula IIa, or a stereoisomer or a tautomer or a pharmaceutically acceptable salt thereof, Ar is phenyl substituted with one to four R₅ groups. In some embodiments of a compound of formula IIa, or a stereoisomer or a tautomer or a pharmaceutically acceptable salt thereof, Ar is phenyl substituted with one to three R₅ groups. In some embodiments of a compound of formula IIa, or a stereoisomer or a tautomer or a pharmaceutically acceptable salt thereof, Ar is phenyl substituted with one or two R₅ groups. In some embodiments of a compound of formula IIa, or a stereoisomer or a tautomer or a pharmaceutically acceptable salt thereof, Ar is phenyl substituted with one R₅ group. In some embodiments of a compound of formula IIa, or a stereoisomer or a tautomer or a pharmaceutically acceptable salt thereof, Ar is phenyl substituted with two R₅ groups.

In some embodiments of a compound of formula IIa, or a stereoisomer or a tautomer or a pharmaceutically acceptable salt thereof, Ar is heteroaryl, optionally substituted with one to four R₅ groups. In some embodiments of a compound of formula IIa, or a stereoisomer or a tautomer or a pharmaceutically acceptable salt thereof, Ar is a 5- to 10-membered heteroaryl ring optionally substituted with one to four R₅ groups. In some embodiments of a compound of formula IIa, or a stereoisomer or a tautomer or a pharmaceutically acceptable salt thereof, Ar is a 5- to 10-membered heteroaryl ring optionally substituted with one to three R₅ groups. In some embodiments of a compound of formula IIa, or a stereoisomer or a tautomer or a pharmaceutically acceptable salt thereof, Ar is a 5- to 10-membered heteroaryl ring optionally substituted with one or two R₅ groups. In some embodiments of a compound of formula IIa, or a stereoisomer or a tautomer or a pharmaceutically acceptable salt thereof, Ar is a 5- to 10-membered heteroaryl ring optionally substituted with two R₅ groups. In some embodiments of a compound of formula IIa, or a stereoisomer or a tautomer or a pharmaceutically acceptable salt thereof, Ar is a 5- to 10-membered heteroaryl ring optionally substituted with one R₅ group.

In some embodiments of a compound of formula IIa, or a stereoisomer or a tautomer or a pharmaceutically acceptable salt thereof, each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇(O)OR₈, NR₇SO₂R₈, optionally substituted aryl-(C₁-C₆ alkylene), optionally substituted aryl, optionally substituted heterocyclyl-(C₁-C₆ alkylene), optionally substituted heterocyclyl, or optionally substituted heteroaryl. In some embodiments of a compound of formula IIa, or a stereoisomer or a tautomer or a pharmaceutically acceptable salt thereof, each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, benzyl, naphthyl, or phenyl, wherein benzyl, naphthyl and phenyl are optionally substituted with one to three groups independently selected from halogen, C₁-C₆ haloalkyl, and C₁-C₆ haloalkoxy. In some embodiments of a compound of formula IIa, or a stereoisomer or a tautomer or a pharmaceutically acceptable salt thereof, each R₅ is independently F, CL, Br, or CF₃.

In some embodiments of a compound of formula IIa, or a stereoisomer or a tautomer or a pharmaceutically acceptable salt thereof, two R₅ groups are linked together to form an optionally substituted 5- or 6-membered aromatic moiety or an optionally substituted 4- to 7-membered non-aromatic cyclic moiety. In further embodiments of a compound of formula IIa, or a stereoisomer or a tautomer or a pharmaceutically acceptable salt thereof, the 5- or 6-membered aromatic moiety and the 4- to 7-membered non-aromatic cyclic moiety are optionally substituted with one or more groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), and optionally substituted heteroaryl. In still further embodiments of a compound of formula IIa, or a stereoisomer or a tautomer or a pharmaceutically acceptable salt thereof, aryl-(C₁-C₆ alkylene), aryl, heterocyclyl-(C₁-C₆ alkylene), and heteroaryl are optionally substituted with one or more groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano.

In some embodiments of a compound of formula IIa, or a stereoisomer or a tautomer or a pharmaceutically acceptable salt thereof, R₃ and R₄ are independently hydrogen, optionally substituted C₁-C₆ alkyl, optionally substituted aryl, C(O)R₇, or C₁-C₆-alkyloxycarbonyl. In some embodiments of a compound of formula IIa, or a stereoisomer or a tautomer or a pharmaceutically acceptable salt thereof, R₃ and R₄ are independently hydrogen, optionally substituted C₁-C₆ alkyl, C(O)R₇, C₁-C₆-alkyloxycarbonyl, or phenyl optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano. In some embodiments of a compound of formula IIa, or a stereoisomer or a tautomer or a pharmaceutically acceptable salt thereof, R₃ and R₄ are independently hydrogen; C(O)R₇; C₁-C₆-alkyloxycarbonyl; C₁-C₆ alkyl optionally substituted with one or more groups independently selected from C₁-C₆ alkyl, C₁-C₆ haloalkyl, C₁-C₆ alkoxy, and C₁-C₆ haloalkoxy; or phenyl optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano. In some embodiments of a compound of formula IIa, or a stereoisomer or a tautomer or a pharmaceutically acceptable salt thereof, R₃ and R₄ are independently hydrogen or C₁-C₆ alkyl. In some embodiments of a compound of formula IIa, or a stereoisomer or a tautomer or a pharmaceutically acceptable salt thereof, R₃ and R₄ are independently hydrogen or C₁-C₃ alkyl. In some embodiments of a compound of formula IIa, or a stereoisomer or a tautomer or a pharmaceutically acceptable salt thereof, R₃ and R₄ are both hydrogen. In some embodiments of a compound of formula IIa, or a stereoisomer or a tautomer or a pharmaceutically acceptable salt thereof, R₃ and R₄ are both methyl. In some embodiments of a compound of formula IIa, or a stereoisomer or a tautomer or a pharmaceutically acceptable salt thereof, R₃ and R₄ are both ethyl.

In some embodiments of a compound of formula IIa, or a stereoisomer or a tautomer or a pharmaceutically acceptable salt thereof, R₃ and R₄, together with the nitrogen to which they are attached, form a 4- to 6-membered heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇. In some embodiments of a compound of formula IIa, or a stereoisomer or a tautomer or a pharmaceutically acceptable salt thereof, R₃ and R₄, together with the nitrogen to which they are attached, form a heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇; wherein the heterocycle is azetidine, pyrrolidine, piperidine, azepane, piperazine, or homopiperazine.

In some embodiments of a compound of formula IIa, or a stereoisomer or a tautomer or a pharmaceutically acceptable salt thereof, each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇(O)OR₈, NR₇SO₂R₈, optionally substituted aryl, or optionally substituted heteroaryl. In some embodiments of a compound of formula IIa, or a stereoisomer or a tautomer or a pharmaceutically acceptable salt thereof, each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, phenyl optionally substituted with one or more groups selected from halogen, C₁-C₆ alkyl, C₁-C₆ haloalkyl, C₁-C₆ alkoxy, C₁-C₆ haloalkoxy, nitro, and cyano, or 5- to 6-membered heteroaryl optionally substituted with one or more groups selected from halogen, C₁-C₆ alkyl, C₁-C₆ haloalkyl, C₁-C₆ alkoxy, and C₁-C₆ haloalkoxy. In some embodiments of a compound of formula IIa, or a stereoisomer or a tautomer or a pharmaceutically acceptable salt thereof, each R₁ is independently halogen, C₁-C₆ alkyl, or C₁-C₆ haloalkyl. In some embodiments of a compound of formula IIa, or a stereoisomer or a tautomer or a pharmaceutically acceptable salt thereof, each R₁ is independently F, Cl, Br, methyl, or ethyl.

In some embodiments of a compound of formula IIa, or a stereoisomer or a tautomer or a pharmaceutically acceptable salt thereof, x is 0, 1, 2, or 3. In some embodiments of a compound of formula IIa, or a stereoisomer or a tautomer or a pharmaceutically acceptable salt thereof, x is 0, 1, or 2. In some embodiments of a compound of formula IIa, or a stereoisomer or a tautomer or a pharmaceutically acceptable salt thereof, x is 0 or 1. In some embodiments of a compound of formula IIa, or a stereoisomer or a tautomer or a pharmaceutically acceptable salt thereof, x is 0. In some embodiments of a compound of formula IIa, or a stereoisomer or a tautomer or a pharmaceutically acceptable salt thereof, x is 1. In some embodiments of a compound of formula IIa, or a stereoisomer or a tautomer or a pharmaceutically acceptable salt thereof, x is 2.

In some embodiments of a compound of formula IIa, or a stereoisomer or a tautomer or a pharmaceutically acceptable salt thereof, two R₁ groups are linked together to form an optionally substituted 5- or 6-membered aromatic moiety or an optionally substituted 4- to 7-membered non-aromatic cyclic moiety. In further embodiments of a compound of formula IIa, or a stereoisomer or a tautomer or a pharmaceutically acceptable salt thereof, the 5- or 6-membered aromatic moiety and the 4- to 7-membered non-aromatic cyclic moiety are optionally substituted with one or more groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), and optionally substituted heteroaryl. In still further embodiments of a compound of formula IIa, or a stereoisomer or a tautomer or a pharmaceutically acceptable salt thereof, aryl-(C₁-C₆ alkylene), aryl, heterocyclyl-(C₁-C₆ alkylene), and heteroaryl are optionally substituted with one or more groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano.

In some embodiments of a compound of formula IIa, or a stereoisomer or a tautomer or a pharmaceutically acceptable salt thereof, L is independently selected from the group consisting of C₁-C₆ alkylene, C₂-C₆ alkenylene, C₂-C₆ alkynylene, —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-. In some embodiments of a compound of formula IIa, or a stereoisomer or a tautomer or a pharmaceutically acceptable salt thereof, L is independently selected from the group consisting of C₁-C₆ alkylene and C₂-C₆ alkenylene. In some embodiments of a compound of formula IIa, or a stereoisomer or a tautomer or a pharmaceutically acceptable salt thereof, L is C₁-C₆ alkylene. In some embodiments of a compound of formula IIa, or a stereoisomer or a tautomer or a pharmaceutically acceptable salt thereof, L is —CH₂—CH₂—. In some embodiments of a compound of formula IIa, or a stereoisomer or a tautomer or a pharmaceutically acceptable salt thereof, L is C₂-C₆ alkenylene. In some embodiments of a compound of formula IIa, or a stereoisomer or a tautomer or a pharmaceutically acceptable salt thereof, L is independently trans-CH═CH— or cis-CH═CH—. In some embodiments of a compound of formula IIa, or a stereoisomer or a tautomer or a pharmaceutically acceptable salt thereof, L is trans-CH═CH—. In some embodiments of a compound of formula IIa, or a stereoisomer or a tautomer or a pharmaceutically acceptable salt thereof, L is cis-CH═CH—. In some embodiments of a compound of formula IIa, or a stereoisomer or a tautomer or a pharmaceutically acceptable salt thereof, L is C₂-C₆ alkynylene. In some embodiments of a compound of formula IIa, or a stereoisomer or a tautomer or a pharmaceutically acceptable salt thereof, L is independently selected from the group consisting of —(C₁-C₆ alkylene)-O—, —(C₁-C₆ alkylene)-NR—, —NR₇—(C₁-C₆ alkylene)-, —O—(C₁-C₆ alkylene)-, —(C₁-C₆ alkylene)-O—(C₁-C₆ alkylene), and —(C₁-C₆ alkylene)-NR₇—(C₁-C₆ alkylene). In some embodiments of a compound of formula IIa, or a stereoisomer or a tautomer or a pharmaceutically acceptable salt thereof, L is independently selected from the group consisting of —(C₁-C₆ alkylene)-O— and —(C₁-C₆ alkylene)-NR₇—. In some embodiments of a compound of formula IIa, or a stereoisomer or a tautomer or a pharmaceutically acceptable salt thereof, L is independently selected from the group consisting of —NR₇C(O)—, —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-. In some embodiments of a compound of formula IIa, or a stereoisomer or a tautomer or a pharmaceutically acceptable salt thereof, L is —C(O)NR₇—. In some embodiments of a compound of formula IIa, or a stereoisomer or a tautomer or a pharmaceutically acceptable salt thereof, L is —C(O)—. In some embodiments of a compound of formula IIa, or a stereoisomer or a tautomer or a pharmaceutically acceptable salt thereof, L is —C(O)C(O)NR₇—. In some embodiments of a compound of formula IIa, or a stereoisomer or a tautomer or a pharmaceutically acceptable salt thereof, L —C(O)NR₇—. In some embodiments of a compound of formula IIa, or a stereoisomer or a tautomer or a pharmaceutically acceptable salt thereof, L is —C(O)NR₇-(alkylene)-. In some embodiments of a compound of formula IIa, or a stereoisomer or a tautomer or a pharmaceutically acceptable salt thereof, L is —C(O)—(C₁-C₆ alkylene)-. In some embodiments of a compound of formula IIa, or a stereoisomer or a tautomer or a pharmaceutically acceptable salt thereof, L is —(C₁-C₆ alkylene)-C(O)—. In some embodiments of a compound of formula IIa, or a stereoisomer or a tautomer or a pharmaceutically acceptable salt thereof, L is —(C₁-C₆ alkylene)-C(O)—(C₁-C₆ alkylene)-.

In some embodiments of a compound of formula IIa, or a stereoisomer or a tautomer or a pharmaceutically acceptable salt thereof, n is 0 or 1. In some embodiments of a compound of formula IIa, or a stereoisomer or a tautomer or a pharmaceutically acceptable salt thereof, n is 1. In some embodiments of a compound of formula IIa, or a stereoisomer or a tautomer or a pharmaceutically acceptable salt thereof, n is 0.

In some embodiments of a compound of formula IIa, or a stereoisomer or a tautomer or a pharmaceutically acceptable salt thereof, B is alkylene interrupted by a carbonyl (C═O) or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy. In some embodiments of a compound of formula IIa, or a stereoisomer or a tautomer or a pharmaceutically acceptable salt thereof, B is alkylene interrupted by a carbonyl (C═O) and optionally substituted by one to four groups, which are independently C₁-C₆ alkyl, C₁-C₆ haloalkyl, C₁-C₆ alkoxy, or C₁-C₆ haloalkoxy. In some embodiments of a compound of formula IIa, or a stereoisomer or a tautomer or a pharmaceutically acceptable salt thereof, B is alkylene interrupted by a sulfonyl group (SO₂), and optionally substituted by one to four groups, which are independently C₁-C₆ alkyl, C₁-C₆ haloalkyl, C₁-C₆ alkoxy, or C₁-C₆ haloalkoxy. In some embodiments of a compound of formula IIa, or a stereoisomer or a tautomer or a pharmaceutically acceptable salt thereof, B is alkylene optionally interrupted by a carbonyl (C═O) or sulfonyl group (SO₂). In some embodiments of a compound of formula IIa, or a stereoisomer or a tautomer or a pharmaceutically acceptable salt thereof, B is alkylene interrupted by a carbonyl (C═O). In some embodiments of a compound of formula IIa, or a stereoisomer or a tautomer or a pharmaceutically acceptable salt thereof, B is alkylene interrupted by a sulfonyl group (SO₂). In some embodiments of a compound of formula IIa, or a stereoisomer or a tautomer or a pharmaceutically acceptable salt thereof, B is alkylene or —C(O)—(C₁-C₆ alkylene)-. In some embodiments of a compound of formula IIa, or a stereoisomer or a tautomer or a pharmaceutically acceptable salt thereof, B is alkylene. In some embodiments of a compound of formula IIa, or a stereoisomer or a tautomer or a pharmaceutically acceptable salt thereof, B is —C(O)—(C₁-C₆ alkylene)-.

In some embodiments of a compound of formula IIa, or a stereoisomer or a tautomer or a pharmaceutically acceptable salt thereof, D is CR₇. In some embodiments of a compound of formula IIa, or a stereoisomer or a tautomer or a pharmaceutically acceptable salt thereof, D is CH. In some embodiments of a compound of formula IIa, or a stereoisomer or a tautomer or a pharmaceutically acceptable salt thereof, D is —C(C₁-C₆ alkyl). In some embodiments of a compound of formula IIa, or a stereoisomer or a tautomer or a pharmaceutically acceptable salt thereof, D is N.

In some embodiments of a compound of formula IIa, or a stereoisomer or a tautomer or a pharmaceutically acceptable salt thereof, D is CH; n is 1; x is 0, 1, or 2; L is trans-CH═CH—; B is an alkylene group; Ar is phenyl substituted with one to four R₅ groups. In some embodiments of a compound of formula IIa, or a stereoisomer or a tautomer or a pharmaceutically acceptable salt thereof, D is CH; n is 1; x is 0, 1, or 2; L is trans-CH═CH—; B is an alkylene group; Ar is phenyl substituted with one to four R₅ groups; R₂ is hydrogen or C₁-C₆ alkyl; and R₃ and R₄ are independently hydrogen or C₁-C₃ alkyl. In some embodiments of a compound of formula IIa, or a stereoisomer or a tautomer or a pharmaceutically acceptable salt thereof, D is CH; n is 1; x is 0, 1, or 2; L is trans-CH═CH—; B is an alkylene group; Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, benzyl, naphthyl, or phenyl, wherein benzyl, naphthyl and phenyl are optionally substituted with one to three groups independently selected from halogen, C₁-C₆ haloalkyl, and C₁-C₆ haloalkoxy; and each R₁ is independently halogen, C₁-C₆ alkyl, or C₁-C₆ haloalkyl.

In some embodiments of a compound of formula IIa, or a stereoisomer or a tautomer or a pharmaceutically acceptable salt thereof, D is CH; n is 1; x is 0, 1, or 2; L is trans-CH═CH—; B is B is alkylene interrupted by a carbonyl (C═O); Ar is phenyl substituted with one to four R₅ groups. In some embodiments of a compound of formula IIa, or a stereoisomer or a tautomer or a pharmaceutically acceptable salt thereof, D is CH; n is 1; x is 0, 1, or 2; L is trans-CH═CH—; B is B is alkylene interrupted by a carbonyl (C═O); Ar is phenyl substituted with one to four R₅ groups; R₂ is hydrogen or C₁-C₆ alkyl; and R₃ and R₄ are independently hydrogen or C₁-C₃ alkyl. In some embodiments of a compound of formula IIa, or a stereoisomer or a tautomer or a pharmaceutically acceptable salt thereof, D is CH; n is 1; x is 0, 1, or 2; L is trans-CH═CH—; B is B is alkylene interrupted by a carbonyl (C═O); Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, benzyl, naphthyl, or phenyl, wherein benzyl, naphthyl and phenyl are optionally substituted with one to three groups independently selected from halogen, C₁-C₆ haloalkyl, and C₁-C₆ haloalkoxy; and each R₁ is independently halogen, C₁-C₆ alkyl, or C₁-C₆ haloalkyl.

In some embodiments of a compound of formula IIa, or a stereoisomer or a tautomer or a pharmaceutically acceptable salt thereof, D is CH; n is 1; x is 0, 1, or 2; L is C₁-C₆ alkylene; B is an alkylene group; Ar is phenyl substituted with one to four R₅ groups. In some embodiments of a compound of formula IIa, or a stereoisomer or a tautomer or a pharmaceutically acceptable salt thereof, D is CH; n is 1; x is 0, 1, or 2; L is C₁-C₆ alkylene; B is an alkylene group; Ar is phenyl substituted with one to four R₅ groups; R₂ is hydrogen or C₁-C₆ alkyl; and R₃ and R₄ are independently hydrogen or C₁-C₃ alkyl. In some embodiments of a compound of formula IIa, or a stereoisomer or a tautomer or a pharmaceutically acceptable salt thereof, D is CH; n is 1; x is 0, 1, or 2; L is C₁-C₆ alkylene; B is an alkylene group; Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, benzyl, naphthyl, or phenyl, wherein benzyl, naphthyl and phenyl are optionally substituted with one to three groups independently selected from halogen, C₁-C₆ haloalkyl, and C₁-C₆ haloalkoxy; and each R₁ is independently halogen, C₁-C₆ alkyl, or C₁-C₆ haloalkyl. In some embodiments of a compound of formula IIa, or a stereoisomer or a tautomer or a pharmaceutically acceptable salt thereof, D is CH; n is 1; x is 0, 1, or 2; L is ethylene; B is an alkylene group; Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, benzyl, naphthyl, or phenyl, wherein benzyl, naphthyl and phenyl are optionally substituted with one to three groups independently selected from halogen, C₁-C₆ haloalkyl, and C₁-C₆ haloalkoxy; and each R₁ is independently halogen, C₁-C₆ alkyl, or C₁-C₆ haloalkyl.

In some embodiments of a compound of formula IIa, or a stereoisomer or a tautomer or a pharmaceutically acceptable salt thereof, D is CH; n is 1; x is 0, 1, or 2; L is —C(O)NR₇—; B is an alkylene group; Ar is phenyl substituted with one to four R₅ groups. In some embodiments of a compound of formula IIa, or a stereoisomer or a tautomer or a pharmaceutically acceptable salt thereof, D is CH; n is 1; x is 0, 1, or 2; L is —C(O)NR₇—; B is an alkylene group; Ar is phenyl substituted with one to four R₅ groups; R₂ is hydrogen or C₁-C₆ alkyl; and R₃ and R₄ are independently hydrogen or C₁-C₃ alkyl. In some embodiments of a compound of formula IIa, or a stereoisomer or a tautomer or a pharmaceutically acceptable salt thereof, D is CH; n is 1; x is 0, 1, or 2; L is —C(O)NR₇—; B is an alkylene group; Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, benzyl, naphthyl, or phenyl, wherein benzyl, naphthyl and phenyl are optionally substituted with one to three groups independently selected from halogen, C₁-C₆ haloalkyl, and C₁-C₆ haloalkoxy; and each R₁ is independently halogen, C₁-C₆ alkyl, or C₁-C₆ haloalkyl.

In some embodiments of a compound of formula IIa, or a stereoisomer or a tautomer or a pharmaceutically acceptable salt thereof, D is CH; n is 0; x is 0, 1, or 2; and B is an alkylene group.

In some embodiments of a compound of formula IIa, or a stereoisomer or a tautomer or a pharmaceutically acceptable salt thereof, D is CH; n is 0; x is 0, 1, or 2; B is an alkylene group; and Ar is phenyl substituted with one to four R₅ groups. In some embodiments of a compound of formula IIa, or a stereoisomer or a tautomer or a pharmaceutically acceptable salt thereof, D is CH; n is 0; x is 0, 1, or 2; B is an alkylene group; Ar is phenyl substituted with one to four R₅ groups; R₂ is hydrogen or C₁-C₆ alkyl; and R₃ and R₄ are independently hydrogen or C₁-C₃ alkyl. In some embodiments of a compound of formula IIa, or a stereoisomer or a tautomer or a pharmaceutically acceptable salt thereof, D is CH; n is 0; x is 0, 1, or 2; B is an alkylene group; Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, benzyl, naphthyl, or phenyl, wherein benzyl, naphthyl and phenyl are optionally substituted with one to three groups independently selected from halogen, C₁-C₆ haloalkyl, and C₁-C₆ haloalkoxy; and each R₁ is independently halogen, C₁-C₆ alkyl, or C₁-C₆ haloalkyl.

In some embodiments of a compound of formula IIa, or a stereoisomer or a tautomer or a pharmaceutically acceptable salt thereof, D is CH; n is 0; x is 0, 1, or 2; B is alkylene interrupted by a carbonyl (C═O); and Ar is phenyl substituted with one to four R₅ groups. In some embodiments of a compound of formula IIa, or a stereoisomer or a tautomer or a pharmaceutically acceptable salt thereof, D is CH; n is 0; x is 0, 1, or 2; B is alkylene interrupted by a carbonyl (C═O); Ar is phenyl substituted with one to four R₅ groups; R₂ is hydrogen or C₁—C₆ alkyl; and R₃ and R₄ are independently hydrogen or C₁-C₃ alkyl. In some embodiments of a compound of formula IIa, or a stereoisomer or a tautomer or a pharmaceutically acceptable salt thereof, D is CH; n is 0; x is 0, 1, or 2; B is alkylene interrupted by a carbonyl (C═O); Ar is phenyl substituted with one to four R₅ groups; R₂ is hydrogen or C₁-C₆ alkyl; R₃ and R₄ are independently hydrogen or C₁-C₃ alkyl; each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, benzyl, naphthyl, or phenyl, wherein benzyl, naphthyl and phenyl are optionally substituted with one to three groups independently selected from halogen, C₁-C₆ haloalkyl, and C₁-C₆ haloalkoxy; and each R₁ is independently halogen, C₁-C₆ alkyl, or C₁-C₆ haloalkyl.

In some embodiments, the invention is a compound of formula IIIa or a stereoisomer or a tautomer or a pharmaceutically acceptable salt thereof.

In some embodiments of a compound of formula IIIa, or a stereoisomer or a tautomer or a pharmaceutically acceptable salt thereof, Ar is aryl optionally substituted with one to five R₅ groups. In some embodiments of a compound of formula IIIa, or a stereoisomer or a tautomer or a pharmaceutically acceptable salt thereof, Ar is phenyl or naphthyl, wherein phenyl and naphthyl are optionally substituted with one to five R₅ groups. In some embodiments of a compound of formula IIIa, or a stereoisomer or a tautomer or a pharmaceutically acceptable salt thereof, Ar is phenyl substituted with one to five R₅ groups. In some embodiments of a compound of formula IIIa, or a stereoisomer or a tautomer or a pharmaceutically acceptable salt thereof, Ar is phenyl substituted with one to four R₅ groups. In some embodiments of a compound of formula IIIa, or a stereoisomer or a tautomer or a pharmaceutically acceptable salt thereof, Ar is phenyl substituted with one to three R₅ groups. In some embodiments of a compound of formula IIIa, or a stereoisomer or a tautomer or a pharmaceutically acceptable salt thereof, Ar is phenyl substituted with one or two R₅ groups.

In some embodiments of a compound of formula IIIa, or a stereoisomer or a tautomer or a pharmaceutically acceptable salt thereof, Ar is heteroaryl, optionally substituted with one to four R₅ groups. In some embodiments of a compound of formula IIIa, or a stereoisomer or a tautomer or a pharmaceutically acceptable salt thereof, Ar is a 5- to 10-membered heteroaryl ring optionally substituted with one to four R₅ groups. In some embodiments of a compound of formula IIIa, or a stereoisomer or a tautomer or a pharmaceutically acceptable salt thereof, Ar is a 5- to 10-membered heteroaryl ring optionally substituted with one to three R₅ groups. In some embodiments of a compound of formula IIIa, or a stereoisomer or a tautomer or a pharmaceutically acceptable salt thereof, Ar is a 5- to 10-membered heteroaryl ring optionally substituted with one or two R₅ groups. In some embodiments of a compound of formula IIIa, or a stereoisomer or a tautomer or a pharmaceutically acceptable salt thereof, Ar is a 5- to 10-membered heteroaryl ring optionally substituted with one R₅ group. In some embodiments of a compound of formula IIIa, or a stereoisomer or a tautomer or a pharmaceutically acceptable salt thereof, Ar is a pyridine or pyridazine optionally substituted with one to four R₅ groups independently selected from halogen and C₁-C₆ alkyl.

In some embodiments of a compound of formula IIIa, or a stereoisomer or a tautomer or a pharmaceutically acceptable salt thereof, each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇(O)OR₈, NR₇SO₂R₈, optionally substituted aryl-(C₁-C₆ alkylene), optionally substituted aryl, optionally substituted heterocyclyl-(C₁-C₆ alkylene), optionally substituted heterocyclyl, or optionally substituted heteroaryl. In some embodiments of a compound of formula IIIa, or a stereoisomer or a tautomer or a pharmaceutically acceptable salt thereof, each R₅ is independently halogen, C₁-C₆ alkyl, C₁-C₆ haloalkyl, C₁-C₆ hydroxyalkyl, C₁-C₆ alkoxy, C₁-C₆ haloalkoxy, cyano, optionally substituted aryl-(C₁-C₆ alkylene), or optionally substituted heterocyclyl-(C₁-C₆ alkylene). In further embodiments of a compound of formula IIIa, or a stereoisomer or a tautomer or a pharmaceutically acceptable salt thereof, aryl and aryl-(C₁-C₆ alkylene) are optionally substituted with one to five groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano; and optionally substituted heterocyclyl-(C₁-C₆ alkylene) is (5- to 6-membered heterocyclyl)-(C₁-C₆ alkylene) optionally substituted with one to four groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano. In some embodiments of a compound of formula IIIa, or a stereoisomer or a tautomer or a pharmaceutically acceptable salt thereof, each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, benzyl, naphthyl, or phenyl, wherein benzyl, naphthyl and phenyl are optionally substituted with one to three groups independently selected from halogen, C₁-C₆ haloalkyl, and C₁-C₆ haloalkoxy. In some embodiments of a compound of formula IIIa, or a stereoisomer or a tautomer or a pharmaceutically acceptable salt thereof, each R₅ is independently halogen, C₁-C₆ haloalkyl, or C₁-C₆ haloalkoxy. In some embodiments of a compound of formula IIIa, or a stereoisomer or a tautomer or a pharmaceutically acceptable salt thereof, each R₅ is independently F, Cl, Br, CF₃, OCF₃, CH₃, cyano, nitro, C(O)N(R₇)₂, NR₇C(O)R₈, or —CH₂OH.

In some embodiments of a compound of formula IIIa, or a stereoisomer or a tautomer or a pharmaceutically acceptable salt thereof, two R₅ groups are linked together to form an optionally substituted 5- or 6-membered aromatic moiety or an optionally substituted 4- to 7-membered non-aromatic cyclic moiety. In further embodiments of a compound of formula IIIa, or a stereoisomer or a tautomer or a pharmaceutically acceptable salt thereof, the 5- or 6-membered aromatic moiety and the 4- to 7-membered non-aromatic cyclic moiety are optionally substituted with one or more groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), and optionally substituted heteroaryl. In still further embodiments of a compound of formula IIIa, or a stereoisomer or a tautomer or a pharmaceutically acceptable salt thereof, aryl-(C₁-C₆ alkylene), aryl, heterocyclyl-(C₁-C₆ alkylene), and heteroaryl are optionally substituted with one or more groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano.

In some embodiments of a compound of formula IIIa, or a stereoisomer or a tautomer or a pharmaceutically acceptable salt thereof, each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇(O)OR₈, NR₇SO₂R₈, optionally substituted aryl, or optionally substituted heteroaryl. In some embodiments of a compound of formula IIIa, or a stereoisomer or a tautomer or a pharmaceutically acceptable salt thereof, each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, phenyl optionally substituted with one or more groups selected from halogen, C₁-C₆ alkyl, C₁-C₆ haloalkyl, C₁-C₆ alkoxy, C₁-C₆ haloalkoxy, nitro, and cyano, or 5- to 6-membered heteroaryl optionally substituted with one or more groups selected from halogen, C₁-C₆ alkyl, C₁-C₆ haloalkyl, C₁-C₆ alkoxy, and C₁-C₆ haloalkoxy. In some embodiments of a compound of formula IIIa, or a stereoisomer or a tautomer or a pharmaceutically acceptable salt thereof, each R₁ is independently halogen, C₁-C₆ alkyl, C₁-C₆ haloalkyl, C₁-C₆ alkoxy, C₁-C₆ haloalkoxy, nitro, cyano, C(O)OR₇, or phenyl optionally substituted with one or more groups selected from halogen, C₁-C₆ alkyl, C₁-C₆ haloalkyl, C₁-C₆ alkoxy, C₁-C₆ haloalkoxy, nitro, and cyano. In some embodiments of a compound of formula IIIa, or a stereoisomer or a tautomer or a pharmaceutically acceptable salt thereof, each R₁ is independently halogen, C₁-C₆ alkyl, or C₁-C₆ haloalkyl. In some embodiments of a compound of formula IIIa, or a stereoisomer or a tautomer or a pharmaceutically acceptable salt thereof, each R₁ is independently F, Cl, Br, CF₃, nitro, cyano, methoxy, methyl, C(O)CH₃, or phenyl optionally substituted with one or more groups selected from halogen, C₁-C₆ alkyl, and C₁-C₆ haloalkyl.

In some embodiments of a compound of formula IIIa, or a stereoisomer or a tautomer or a pharmaceutically acceptable salt thereof, two R₁ groups are linked together to form an optionally substituted 5- or 6-membered aromatic moiety or an optionally substituted 4- to 7-membered non-aromatic cyclic moiety. In further embodiments of a compound of formula IIIa, or a stereoisomer or a tautomer or a pharmaceutically acceptable salt thereof, the 5- or 6-membered aromatic moiety and the 4- to 7-membered non-aromatic cyclic moiety are optionally substituted with one or more groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), and optionally substituted heteroaryl. In still further embodiments of a compound of formula IIIa, or a stereoisomer or a tautomer or a pharmaceutically acceptable salt thereof, aryl-(C₁-C₆ alkylene), aryl, heterocyclyl-(C₁-C₆ alkylene), and heteroaryl are optionally substituted with one or more groups independently selected from halogen, C₁-C₆ alkyl, C₁-C₆ haloalkyl, C₁-C₆ hydroxyalkyl, C₁-C₆ alkoxy, C₁-C₆ haloalkoxy, nitro, and cyano.

In some embodiments of a compound of formula IIIa, or a stereoisomer or a tautomer or a pharmaceutically acceptable salt thereof, x is 0, 1, 2, or 3. In some embodiments of a compound of formula IIIa, or a stereoisomer or a tautomer or a pharmaceutically acceptable salt thereof, x is 0, 1, or 2. In some embodiments of a compound of formula IIIa, or a stereoisomer or a tautomer or a pharmaceutically acceptable salt thereof, x is 0 or 1. In some embodiments of a compound of formula IIIa, or a stereoisomer or a tautomer or a pharmaceutically acceptable salt thereof, x is 0. In some embodiments of a compound of formula IIIa, or a stereoisomer or a tautomer or a pharmaceutically acceptable salt thereof, x is 1. In some embodiments of a compound of formula IIIa, or a stereoisomer or a tautomer or a pharmaceutically acceptable salt thereof, x is 2.

In some embodiments of a compound of formula IIIa, or a stereoisomer or a tautomer or a pharmaceutically acceptable salt thereof, L is independently selected from the group consisting of C₁-C₆ alkylene, C₂-C₆ alkenylene, C₂-C₆ alkynylene, —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-. In some embodiments of a compound of formula IIIa, or a stereoisomer or a tautomer or a pharmaceutically acceptable salt thereof, L is independently selected from the group consisting of C₁-C₆ alkylene and C₂-C₆ alkenylene. In some embodiments of a compound of formula IIIa, or a stereoisomer or a tautomer or a pharmaceutically acceptable salt thereof, L is C₁-C₆ alkylene. In some embodiments of a compound of formula IIIa, or a stereoisomer or a tautomer or a pharmaceutically acceptable salt thereof, L is —CH₂—CH₂—. In some embodiments of a compound of formula IIIa, or a stereoisomer or a tautomer or a pharmaceutically acceptable salt thereof, L is C₂-C₆ alkenylene. In some embodiments of a compound of formula IIIa, or a stereoisomer or a tautomer or a pharmaceutically acceptable salt thereof, L is independently trans-CH═CH— or cis-CH═CH—. In some embodiments of a compound of formula IIIa, or a stereoisomer or a tautomer or a pharmaceutically acceptable salt thereof, L is trans-CH═CH—. In some embodiments of a compound of formula IIIa, or a stereoisomer or a tautomer or a pharmaceutically acceptable salt thereof, L is cis-CH═CH—. In some embodiments of a compound of formula IIIa, or a stereoisomer or a tautomer or a pharmaceutically acceptable salt thereof, L is C₂-C₆ alkynylene. In some embodiments of a compound of formula IIIa, or a stereoisomer or a tautomer or a pharmaceutically acceptable salt thereof, L is independently selected from the group consisting of —(C₁-C₆ alkylene)-O—, —(C₁-C₆ alkylene)-NR—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-. In some embodiments of a compound of formula IIIa, or a stereoisomer or a tautomer or a pharmaceutically acceptable salt thereof, L is independently selected from the group consisting of —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), and —(C₁-C₆ alkylene)-NR₇—(C₁-C₆ alkylene). In some embodiments of a compound of formula IIIa, or a stereoisomer or a tautomer or a pharmaceutically acceptable salt thereof, L is independently selected from the group consisting of —(CH₂)NH—, —C(O)—, —C(O)C(O)NH—, —C(O)NH—, —C(O)NH(CH₂)—, and —C(O)(CH₂)—. In some embodiments of a compound of formula IIIa, or a stereoisomer or a tautomer or a pharmaceutically acceptable salt thereof, L is —(CH₂)NH. In some embodiments of a compound of formula IIIa, or a stereoisomer or a tautomer or a pharmaceutically acceptable salt thereof, L is —C(O)—. In some embodiments of a compound of formula IIIa, or a stereoisomer or a tautomer or a pharmaceutically acceptable salt thereof, L is —C(O)C(O)NH—. In some embodiments of a compound of formula IIIa, or a stereoisomer or a tautomer or a pharmaceutically acceptable salt thereof, L is —C(O)NH—. In some embodiments of a compound of formula IIIa, or a stereoisomer or a tautomer or a pharmaceutically acceptable salt thereof, L is —C(O)NH(CH₂)—. In some embodiments of a compound of formula IIIa, or a stereoisomer or a tautomer or a pharmaceutically acceptable salt thereof, L is —C(O)(CH₂)—.

In some embodiments of a compound of formula IIIa, or a stereoisomer or a tautomer or a pharmaceutically acceptable salt thereof, n is 0 or 1. In some embodiments of a compound of formula IIIa, or a stereoisomer or a tautomer or a pharmaceutically acceptable salt thereof, n is 1. In some embodiments of a compound of formula IIIa, or a stereoisomer or a tautomer or a pharmaceutically acceptable salt thereof, n is 0.

In some embodiments of a compound of formula IIIa, or a stereoisomer or a tautomer or a pharmaceutically acceptable salt thereof, R₆ is hydrogen, C₁-C₆ alkyl, C₁-C₆ haloalkyl, optionally substituted benzyl, or C₁-C₆-carboxyalkyl. In some embodiments of a compound of formula IIIa, or a stereoisomer or a tautomer or a pharmaceutically acceptable salt thereof, R₆ is hydrogen, C₁-C₆ alkyl, C₁-C₆ haloalkyl, C₁-C₆-carboxyalkyl, or benzyl optionally substituted with one to five groups independently selected from halogen, C₁-C₆ alkyl, C₁-C₆ haloalkyl, C₁-C₆ alkoxy, C₁-C₆ haloalkoxy, nitro, and cyano. In some embodiments of a compound of formula IIIa, or a stereoisomer or a tautomer or a pharmaceutically acceptable salt thereof, R₆ is hydrogen, C₁-C₆ alkyl, C₁-C₆ haloalkyl, C₁-C₆-carboxyalkyl, or benzyl optionally substituted with one to five halogens.

In some embodiments of a compound of formula IIIa, or a stereoisomer or a tautomer or a pharmaceutically acceptable salt thereof, D is CR₇. In some embodiments of a compound of formula IIIa, or a stereoisomer or a tautomer or a pharmaceutically acceptable salt thereof, D is CH. In some embodiments of a compound of formula IIIa, or a stereoisomer or a tautomer or a pharmaceutically acceptable salt thereof, D is —C(C₁-C₆ alkyl). In some embodiments of a compound of formula IIIa, or a stereoisomer or a tautomer or a pharmaceutically acceptable salt thereof, D is N.

In some embodiments of a compound of formula IIIa, or a stereoisomer or a tautomer or a pharmaceutically acceptable salt thereof, D is CH; n is 1; x is 0, 1, or 2; L is trans-CH═CH—; Ar is phenyl substituted with one to four R₅ groups; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is trans-CH═CH—; Ar is phenyl substituted with one to four R₅ groups; R₂ is hydrogen or C₁-C₆ alkyl; and R₃ and R₄ are independently hydrogen or C₁-C₃ alkyl; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is trans-CH═CH—; Ar is phenyl substituted with one to four R₅ groups; R₂ is hydrogen or C₁-C₆ alkyl; R₃ and R₄ are independently hydrogen or C₁-C₃ alkyl; R₆ is hydrogen, C₁-C₆ alkyl, C₁-C₆ haloalkyl, C₁-C₆-carboxyalkyl, or benzyl optionally substituted with one to five halogens; each R₅ is independently F, Cl, Br, CF₃, OCF₃, CH₃, cyano, nitro, C(O)N(R₇)₂, NR₇C(O)R₈, or —CH₂OH; and each R₁ is independently F, Cl, Br, CF₃, nitro, cyano, methoxy, methyl, C(O)CH₃, or phenyl optionally substituted with one or more groups selected from halogen, C₁-C₆ alkyl, and C₁-C₆ haloalkyl.

In some embodiments of a compound of formula IIIa, or a stereoisomer or a tautomer or a pharmaceutically acceptable salt thereof, D is CH; n is 1; x is 0, 1, or 2; L is C₁-C₆ alkylene; Ar is phenyl substituted with one to four R₅ groups; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is C₁-C₆ alkylene; Ar is phenyl substituted with one to four R₅ groups; R₂ is hydrogen or C₁-C₆ alkyl; and R₃ and R₄ are independently hydrogen or C₁-C₃ alkyl; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is C₁-C₆ alkylene; Ar is phenyl substituted with one to four R₅ groups; R₂ is hydrogen or C₁-C₆ alkyl; R₃ and R₄ are independently hydrogen or C₁-C₃ alkyl; R₆ is hydrogen, C₁-C₆ alkyl, C₁-C₆ haloalkyl, C₁-C₆-carboxyalkyl, or benzyl optionally substituted with one to five halogens; each R₅ is independently F, Cl, Br, CF₃, OCF₃, CH₃, cyano, nitro, C(O)N(R₇)₂, NR₇C(O)R₈, or —CH₂OH; and each R₁ is independently F, Cl, Br, CF₃, nitro, cyano, methoxy, methyl, C(O)CH₃, or phenyl optionally substituted with one or more groups selected from halogen, C₁-C₆ alkyl, and C₁-C₆ haloalkyl.

In some embodiments of a compound of formula IIIa, or a stereoisomer or a tautomer or a pharmaceutically acceptable salt thereof, D is CH; n is 1; x is 0, 1, or 2; L is —C(O)NR₇—; Ar is phenyl substituted with one to four R₅ groups; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is —C(O)NR₇—; Ar is phenyl substituted with one to four R₅ groups; R₂ is hydrogen or C₁-C₆ alkyl; and R₃ and R₄ are independently hydrogen or C₁-C₃ alkyl; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is —C(O)NR₇—; Ar is phenyl substituted with one to four R₅ groups; R₂ is hydrogen or C₁-C₆ alkyl; R₃ and R₄ are independently hydrogen or C₁-C₃ alkyl; R₆ is hydrogen, C₁-C₆ alkyl, C₁-C₆ haloalkyl, C₁-C₆-carboxyalkyl, or benzyl optionally substituted with one to five halogens; each R₅ is independently F, Cl, Br, CF₃, OCF₃, CH₃, cyano, nitro, C(O)N(R₇)₂, NR₇C(O)R₈, or —CH₂OH; and each R₁ is independently F, Cl, Br, CF₃, nitro, cyano, methoxy, methyl, C(O)CH₃, or phenyl optionally substituted with one or more groups selected from halogen, C₁-C₆ alkyl, and C₁-C₆ haloalkyl.

In some embodiments of a compound of formula IIIa, or a stereoisomer or a tautomer or a pharmaceutically acceptable salt thereof, D is CH; n is 1; x is 0, 1, or 2; L is —C(O)NR₇-(alkylene)-; Ar is phenyl substituted with one to four R₅ groups; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is —C(O)NR₇-(alkylene)-; Ar is phenyl substituted with one to four R₅ groups; R₂ is hydrogen or C₁-C₆ alkyl; and R₃ and R₄ are independently hydrogen or C₁-C₃ alkyl; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is —C(O)NR₇-(alkylene)-; Ar is phenyl substituted with one to four R₅ groups; R₂ is hydrogen or C₁-C₆ alkyl; R₃ and R₄ are independently hydrogen or C₁-C₃ alkyl; R₆ is hydrogen, C₁-C₆ alkyl, C₁-C₆ haloalkyl, C₁-C₆-carboxyalkyl, or benzyl optionally substituted with one to five halogens; each R₅ is independently F, Cl, Br, CF₃, OCF₃, CH₃, cyano, nitro, C(O)N(R₇)₂, NR₇C(O)R₈, or —CH₂OH; and each R₁ is independently F, Cl, Br, CF₃, nitro, cyano, methoxy, methyl, C(O)CH₃, or phenyl optionally substituted with one or more groups selected from halogen, C₁-C₆ alkyl, and C₁-C₆ haloalkyl.

In some embodiments of a compound of formula IIIa, or a stereoisomer or a tautomer or a pharmaceutically acceptable salt thereof, D is CH; n is 1; x is 0, 1, or 2; L is —(C₁-C₆ alkylene)-NR₇—; Ar is phenyl substituted with one to four R₅ groups; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is —(C₁-C₆ alkylene)-NR₇—; Ar is phenyl substituted with one to four R₅ groups; R₂ is hydrogen or C₁-C₆ alkyl; and R₃ and R₄ are independently hydrogen or C₁-C₃ alkyl; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is —(C₁-C₆ alkylene)-NR₇—; Ar is phenyl substituted with one to four R₅ groups; R₂ is hydrogen or C₁-C₆ alkyl; R₃ and R₄ are independently hydrogen or C₁-C₃ alkyl; R₆ is hydrogen, C₁-C₆ alkyl, C₁-C₆ haloalkyl, C₁-C₆-carboxyalkyl, or benzyl optionally substituted with one to five halogens; each R₅ is independently F, Cl, Br, CF₃, OCF₃, CH₃, cyano, nitro, C(O)N(R₇)₂, NR₇C(O)R₈, or —CH₂OH; and each R₁ is independently F, Cl, Br, CF₃, nitro, cyano, methoxy, methyl, C(O)CH₃, or phenyl optionally substituted with one or more groups selected from halogen, C₁-C₆ alkyl, and C₁-C₆ haloalkyl.

In some embodiments of a compound of formula IIIa, or a stereoisomer or a tautomer or a pharmaceutically acceptable salt thereof, D is CH; n is 1; x is 0, 1, or 2; L is —C(O)—(C₁-C₆ alkylene)-; Ar is phenyl substituted with one to four R₅ groups; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is —C(O)—(C₁-C₆ alkylene)-; Ar is phenyl substituted with one to four R₅ groups; R₂ is hydrogen or C₁-C₆ alkyl; and R₃ and R₄ are independently hydrogen or C₁-C₃ alkyl; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is —C(O)—(C₁-C₆ alkylene)-; Ar is phenyl substituted with one to four R₅ groups; R₂ is hydrogen or C₁-C₆ alkyl; R₃ and R₄ are independently hydrogen or C₁-C₃ alkyl; R₆ is hydrogen, C₁-C₆ alkyl, C₁-C₆ haloalkyl, C₁-C₆-carboxyalkyl, or benzyl optionally substituted with one to five halogens; each R₅ is independently F, Cl, Br, CF₃, OCF₃, CH₃, cyano, nitro, C(O)N(R₇)₂, NR₇C(O)R₈, or —CH₂OH; and each R₁ is independently F, Cl, Br, CF₃, nitro, cyano, methoxy, methyl, C(O)CH₃, or phenyl optionally substituted with one or more groups selected from halogen, C₁-C₆ alkyl, and C₁-C₆ haloalkyl.

In some embodiments of a compound of formula IIIa, or a stereoisomer or a tautomer or a pharmaceutically acceptable salt thereof, D is CH; n is 1; x is 0, 1, or 2; L is —C(O)—; Ar is phenyl substituted with one to four R₅ groups; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is —C(O)—; Ar is phenyl substituted with one to four R₅ groups; R₂ is hydrogen or C₁-C₆ alkyl; and R₃ and R₄ are independently hydrogen or C₁-C₃ alkyl; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 1; x is 0, 1, or 2; L is —C(O)—; Ar is phenyl substituted with one to four R₅ groups; R₂ is hydrogen or C₁-C₆ alkyl; R₃ and R₄ are independently hydrogen or C₁-C₃ alkyl; R₆ is hydrogen, C₁-C₆ alkyl, C₁-C₆ haloalkyl, C₁-C₆-carboxyalkyl, or benzyl optionally substituted with one to five halogens; each R₅ is independently F, Cl, Br, CF₃, OCF₃, CH₃, cyano, nitro, C(O)N(R₇)₂, NR₇C(O)R₈, or —CH₂OH; and each R₁ is independently F, Cl, Br, CF₃, nitro, cyano, methoxy, methyl, C(O)CH₃, or phenyl optionally substituted with one or more groups selected from halogen, C₁-C₆ alkyl, and C₁-C₆ haloalkyl.

In some embodiments of a compound of formula IIIa, or a stereoisomer or a tautomer or a pharmaceutically acceptable salt thereof, D is CH; n is 0; x is 0, 1, or 2; Ar is phenyl substituted with one to four R₅ groups; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 0; x is 0, 1, or 2; Ar is phenyl substituted with one to four R₅ groups; R₂ is hydrogen or C₁-C₆ alkyl; and R₃ and R₄ are independently hydrogen or C₁-C₃ alkyl; and R₆ is hydrogen, C₁-C₆ alkyl, C₁-C₆ haloalkyl, C₁-C₆-carboxyalkyl, or benzyl optionally substituted with one to five halogens. In some embodiments of a compound of formula IIIa, or a stereoisomer or a tautomer or a pharmaceutically acceptable salt thereof, D is CH; n is 0; x is 0, 1, or 2; Ar is phenyl substituted with one to four R₅ groups; R₂ is hydrogen or C₁-C₆ alkyl; R₃ and R₄ are independently hydrogen or C₁-C₃ alkyl; R₆ is hydrogen, C₁-C₆ alkyl, C₁-C₆ haloalkyl, C₁-C₆-carboxyalkyl, or benzyl optionally substituted with one to five halogens; each R₅ is independently F, Cl, Br, CF₃, OCF₃, CH₃, cyano, nitro, C(O)N(R₇)₂, NR₇C(O)R₈, or —CH₂OH; and each R₁ is independently F, Cl, Br, CF₃, nitro, cyano, methoxy, methyl, C(O)CH₃, or phenyl optionally substituted with one or more groups selected from halogen, C₁-C₆ alkyl, and C₁-C₆ haloalkyl.

In one embodiment, the present invention is a compound of formula I, formula II, or formula III:

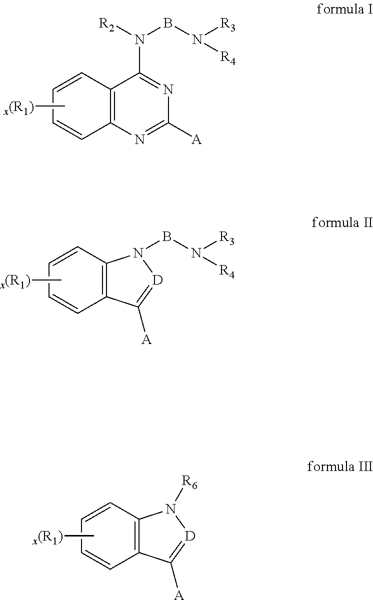

wherein,

A is one of the following groups:

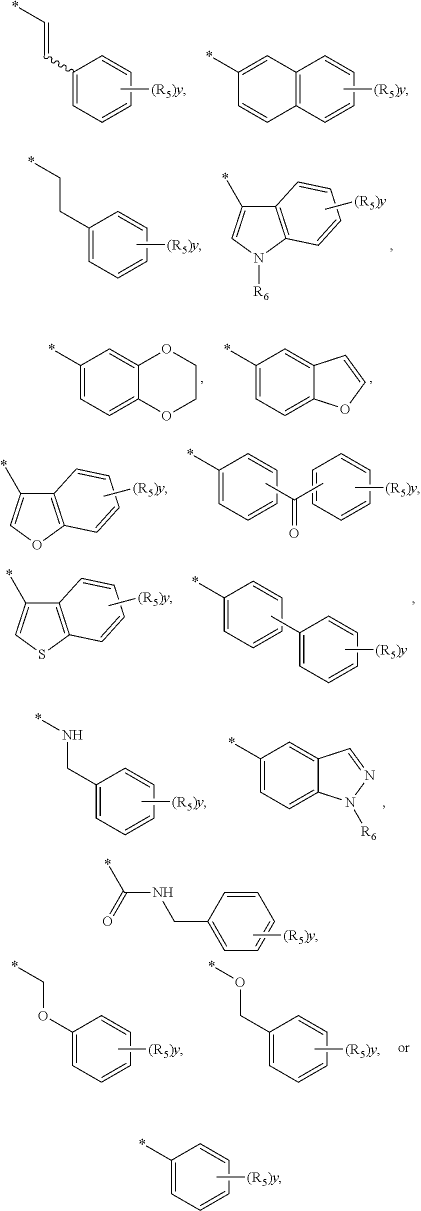

wherein * indicates the point of attachment of group A,

B is an alkylene group, optionally interrupted by a carbonyl (C═O) or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,

D is CH or a heteroatom,

each R₁ is independently halogen, C₁-C₆ alkyl, C₁-C₆ haloalkyl, nitro, or C₁-C₆-alkylamido,

each R₅ is independently halogen, C₁-C₆ alkyl, or C₁-C₆ haloalkyl,

R₂, R₃ and R₄ are independently hydrogen or optionally substituted C₁-C₆ alkyl, or C₁-C₆-alkyloxycarbonyl,

R₆ is hydrogen, optionally substituted C₁-C₆ alkyl, or C₁-C₆-alkyloxycarbonyl,

the wavy bond of formula A indicates that the double bond may be in a cis or trans configuration,

x is 0, 1, 2, 3, or 4,

y is 0, 1, 2, 3, 4, or 5, wherein both x and y are not simultaneously 0, and

and tautomers or pharmaceutically acceptable salts thereof.

In one embodiment, the invention is a compound of formula I and tautomers or pharmaceutically acceptable salts thereof.

In some embodiments, the invention is a compound of formula I, wherein:

A is one of the following groups:

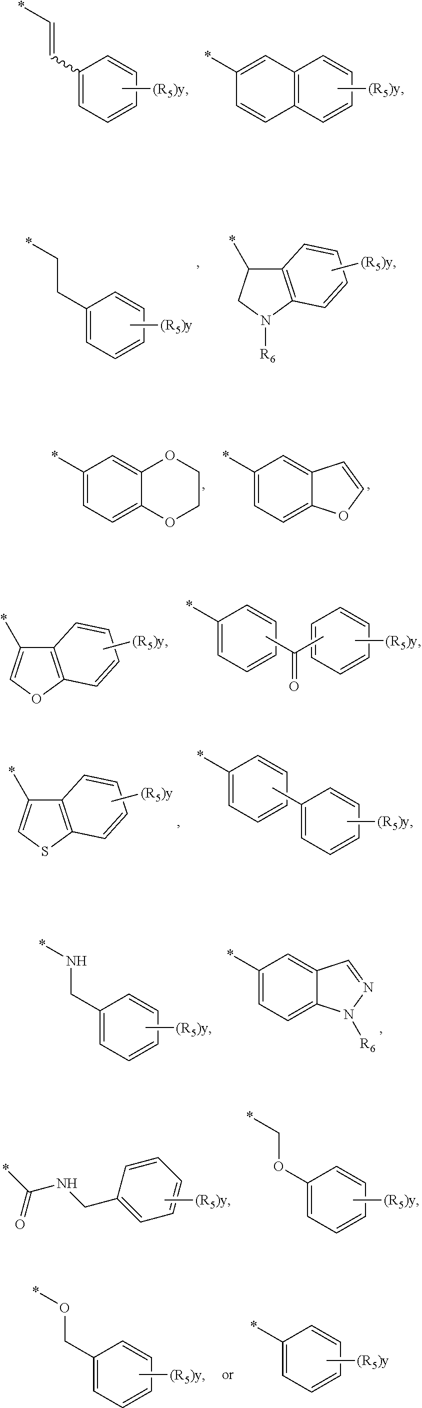

wherein * indicates the point of attachment of group A,

B is a methylene, ethylene, propylene, or butylene group, optionally interrupted by a carbonyl (C═O) or sulfonyl group (SO₂), and optionally substituted by one to four groups, which are independently C₁-C₄ alkyl, C₁-C₄ haloalkyl, C₁-C₄ alkoxy, or C₁-C₄ haloalkoxy,

each R₁ is independently halogen, C₁-C₄ alkyl, C₁-C₄ haloalkyl, nitro, or C₁-C₄-alkylamido,

each R₅ is independently halogen, C₁-C₄ alkyl, or C₁-C₄ haloalkyl,

R₂, R₃ and R₄ are independently hydrogen or optionally substituted C₁-C₄ alkyl, or C₁-C₄-alkyloxycarbonyl,

R₆ is hydrogen, optionally substituted C₁-C₄ alkyl, or C₁-C₄-alkyloxycarbonyl,

the wavy bond of formula A indicates that the double bond may be in a cis or trans configuration,

x is 1, 2, 3, or 4,

y is 1, 2, 3, 4, or 5,

or tautomers or pharmaceutically acceptable salts thereof.

In some embodiments, the invention is a compound of formula I, wherein:

A is one of the following groups:

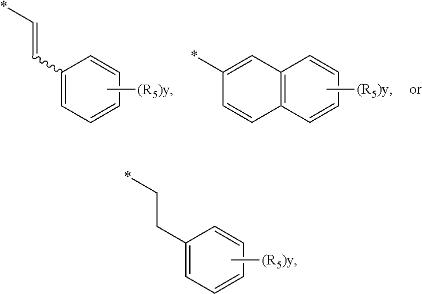

wherein * indicates the point of attachment of group A,

B is (CH₂)₃, optionally substituted by one to three C₁-C₆ alkyl groups, and

or tautomers or pharmaceutically acceptable salts thereof.

In some embodiments, the invention is a compound of formula I, wherein:

A is one of the following groups:

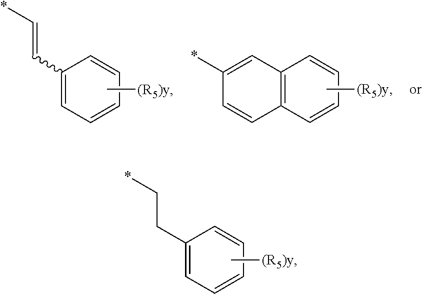

wherein * indicates the point of attachment of group A,

B is (CH₂)₃, optionally substituted by one or two C₁-C₃ alkyl groups,

each R₁ is chloro, fluoro, C₁-C₃ alkyl or C₁-C₃ haloalkyl,

x is 1 or 2,

each R₅ is halogen,

y is 1, 2 or 3,

R₂ is hydrogen,

R₃ and R₄ are independently C₁-C₃ alkyl,

or tautomers or pharmaceutically acceptable salts thereof.

In some embodiments, the invention is a compound of formula I, wherein:

A is one of the following groups:

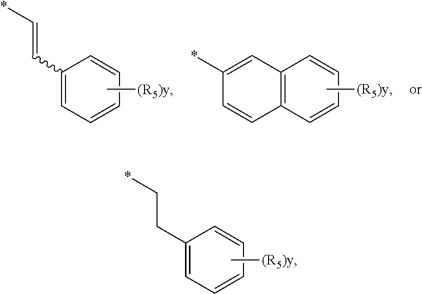

wherein * indicates the point of attachment of group A,

B is an ethylene, propylene, or butylene group, optionally substituted by one to four groups, which are independently C₁-C₃ alkyl, C₁-C₃ haloalkyl, C₁-C₃ alkoxy, or C₁-C₃ haloalkoxy,

each R₁ is independently halogen, C₁-C₃ alkyl, C₁-C₃ haloalkyl, nitro, or C₁-C₃-alkylamido,

each R₅ is independently halogen, C₁-C₃ alkyl, or C₁-C₃ haloalkyl,

R₂, R₃ and R₄ are independently hydrogen or optionally substituted C₁-C₃ alkyl, or C₁-C₃-alkyloxycarbonyl,

R₆ is hydrogen, optionally substituted C₁-C₃ alkyl, or C₁-C₃-alkyloxycarbonyl,

the wavy bond of formula A indicates that the double bond may be in a cis or trans configuration,

x is 1, 2, or 3,

y is 1, 2, or 3,

or tautomers or pharmaceutically acceptable salts thereof.

In some embodiments, the invention is a compound of formula I, wherein:

A is one of the following groups:

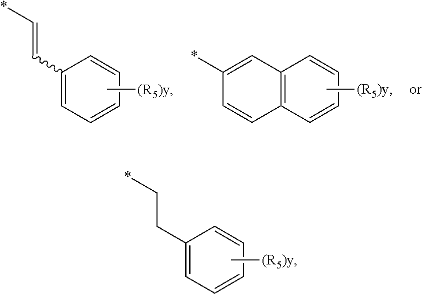

wherein * indicates the point of attachment of group A,

B is a (CH₂)₃ or (CH₂)₄ group, optionally substituted by one to four groups, which are independently methyl, ethyl, methyl or ethyl substituted by one to four chloro or fluoro groups, methoxy, ethoxy, methoxy or ethoxy substituted by one to four chloro or fluoro groups,

each R₁ is independently fluoro, chloro, bromo, methyl, ethyl, methyl or ethyl substituted by one to four chloro or fluoro groups, nitro, acetamido, propanamido,

each R₅ is independently fluoro, chloro, bromo, methyl, ethyl, methyl or ethyl substituted by one to four chloro or fluoro groups,

R₂, R₃ and R₄ are independently hydrogen, methyl, ethyl, methoxycarbonyl, or butoxycarbonyl,

R₆ is hydrogen, methyl, ethyl, benzyl, methoxycarbonyl, or butoxycarbonyl,

the double bond is in a trans configuration,

x is 1 or 2,

y is 1, 2, or 3,

or tautomers or pharmaceutically acceptable salts thereof.

In some embodiments, the invention is a compound of formula I, wherein:

A is one of the following groups:

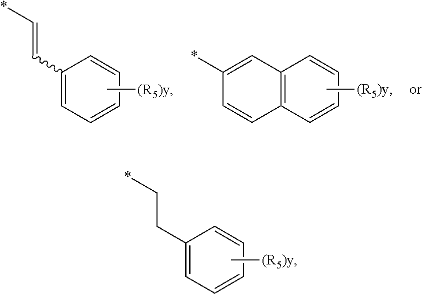

wherein * indicates the point of attachment of group A,

B is a (CH₂)₃ or (CH₂)₄ group, optionally substituted by one to four groups, which are independently methyl, ethyl, methyl or ethyl substituted by one to two chloro or fluoro groups, methoxy, ethoxy, methoxy or ethoxy substituted by one to two chloro or fluoro groups,

each R₁ is independently fluoro, chloro, bromo, methyl, ethyl, CH₂Cl, CHCl₂, CH₂F, CHF₂, CF₃, CH₂CF₃, CF₂CF₃, nitro, acetamido, propanamido,

each R₅ is independently fluoro, chloro, bromo, methyl, ethyl CH₂Cl, CHCl₂, CH₂F, CHF₂, CF₃, CH₂CF₃, or CF₂CF₃,

R₂, R₃ and R₄ are independently hydrogen, methyl, ethyl, or butoxycarbonyl,

R₆ is hydrogen, methyl, ethyl, benzyl, or butoxycarbonyl,

the double bond is in a trans configuration,

x is 1 or 2,

y is 1, 2, or 3,

or tautomers or pharmaceutically acceptable salts thereof.

In some embodiments, the invention is a compound of formula I, wherein:

A is one of the following groups:

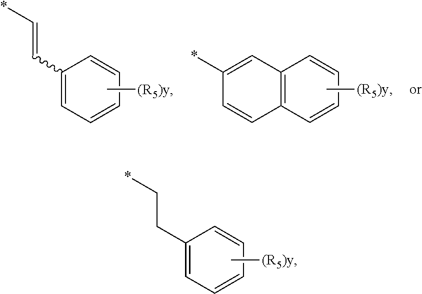

wherein * indicates the point of attachment of group A,

B is a (CH₂)₃ or (CH₂)₄ group, optionally substituted by one to four groups, which are independently methyl or ethyl,

each R₁ is independently fluoro, chloro, bromo, methyl, ethyl, CF₃, CF₂CF₃, nitro, or acetamido,

each R₅ is independently fluoro, chloro, bromo, methyl, ethyl, or CF₃,

R₂, R₃ and R₄ are independently hydrogen, methyl, ethyl, or butoxycarbonyl,

R₆ is hydrogen, methyl, ethyl, benzyl, or butoxycarbonyl,

the double bond is in a trans configuration,

x is 1 or 2,

y is 1, 2, or 3,

or tautomers or pharmaceutically acceptable salts thereof.

In some embodiments, the invention is a compound of formula I, wherein:

A is one of the following groups:

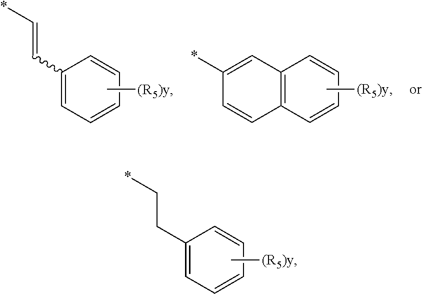

wherein * indicates the point of attachment of group A,

B is a (CH₂)₃ or (CH₂)₄ group, optionally substituted by one or two methyl or ethyl groups,

each R₁ is independently fluoro, chloro, bromo, CF₃, nitro, or acetamido,

each R₅ is independently fluoro, chloro, bromo, or CF₃,

R₂, R₃ and R₄ are independently hydrogen, methyl, ethyl, or butoxycarbonyl,

R₆ is hydrogen, methyl, ethyl, benzyl, or butoxycarbonyl,

the double bond is in a trans configuration,

x is 1 or 2,

y is 1, 2, or 3,

or tautomers or pharmaceutically acceptable salts thereof.

In some embodiments, the invention is a compound of formula I, wherein:

A is one of the following groups:

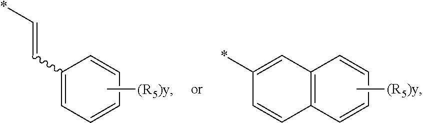

wherein * indicates the point of attachment of group A,

B is (CH₂)₃, substituted by one or two methyl or ethyl groups,

each R₁ is chloro, fluoro, methyl or trifluoromethyl,

x is 1 or 2,

each R₅ is fluoro or chloro,

y is 2 or 3,

R₂ is hydrogen,

R₃ and R₄ are independently methyl or ethyl,

R₆ and R₇ are hydrogen,

or tautomers or pharmaceutically acceptable salts thereof.

In some embodiments, the invention is a compound of formula I,

wherein A is one of the following groups:

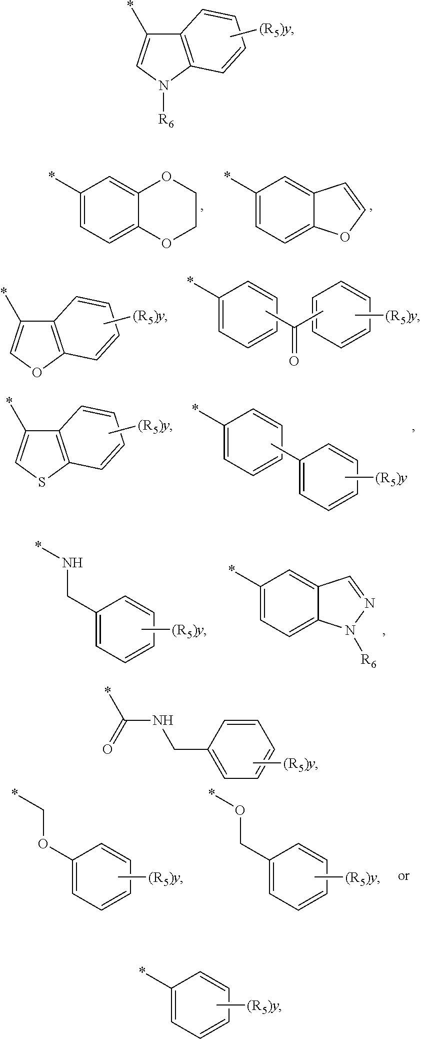

B is (CH₂)₃ or (CH₂)₄, substituted by one or two methyl groups,

each R₁ is chloro, bromo, nitro, or trifluoromethyl,

x is 1 or 2,

each R₅ is independently fluoro or chloro,

y is 2 or 3,

R₂ is hydrogen or methyl,

R₃ and R₄ are independently hydrogen, methyl, or ethyl,

and tautomers or pharmaceutically acceptable salts thereof.

In one embodiment, the invention is a compound of formula I,

wherein A is one of the following groups:

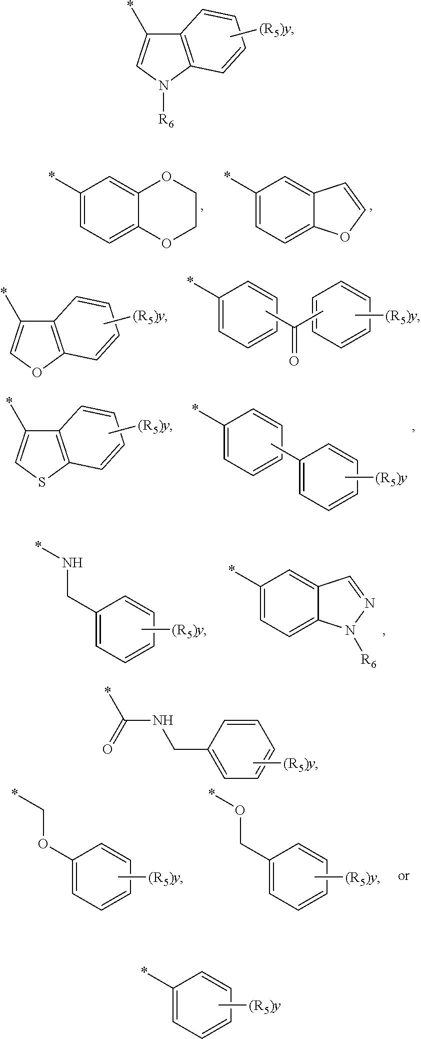

wherein * indicates the point of attachment of group A,

B is CH₂, (CH₂)₃, or CH(CH₃)(CH₂)₃, wherein B is substituted by one or two methyl groups,

each R₁ is chloro, bromo, nitro, or trifluoromethyl,

x is 1 or 2,

each R₅ is independently fluoro or chloro,

y is 2 or 3,

R₂ is methyl,

R₃ and R₄ are independently methyl or ethyl,

and tautomers or pharmaceutically acceptable salts thereof.

In one embodiment, the invention is a compound of formula I,

wherein A is one of the following groups:

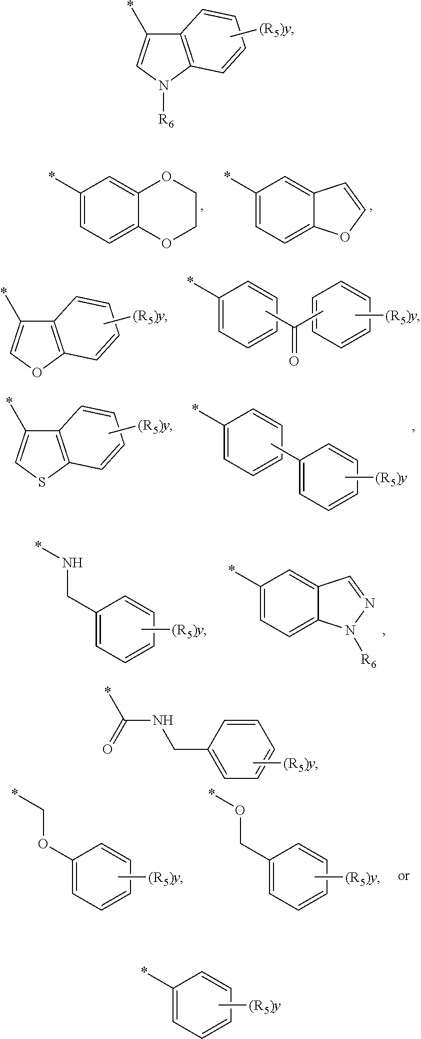

wherein * indicates the point of attachment of group A,

B is CH₂, (CH₂)₃, Or CH(CH₃)(CH₂)₃, wherein B is substituted by one or two methyl groups,

each R₁ is chloro, bromo, nitro, or trifluoromethyl,

x is 1 or 2,

y is 0,

R₂ is methyl,

R₃ and R₄ are independently methyl or ethyl,

and tautomers or pharmaceutically acceptable salts thereof.

In one embodiment, the invention is a compound of formula I,

wherein A is one of the following groups:

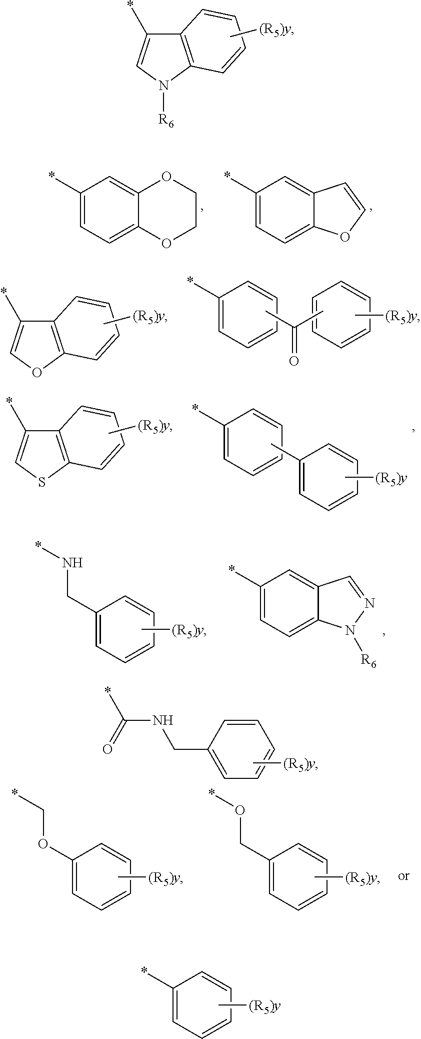

B is (CH₂)₃ or CH(CH₃)(CH₂)₃,

each R₁ is chloro, bromo, nitro, or trifluoromethyl,

x is 1 or 2,

y is 0,

R₂ is hydrogen or methyl,

R₃ and R₄ are independently hydrogen, methyl, or ethyl,

and tautomers or pharmaceutically acceptable salts thereof.

In one embodiment, the invention is a compound of formula I,

wherein A is one of the following groups:

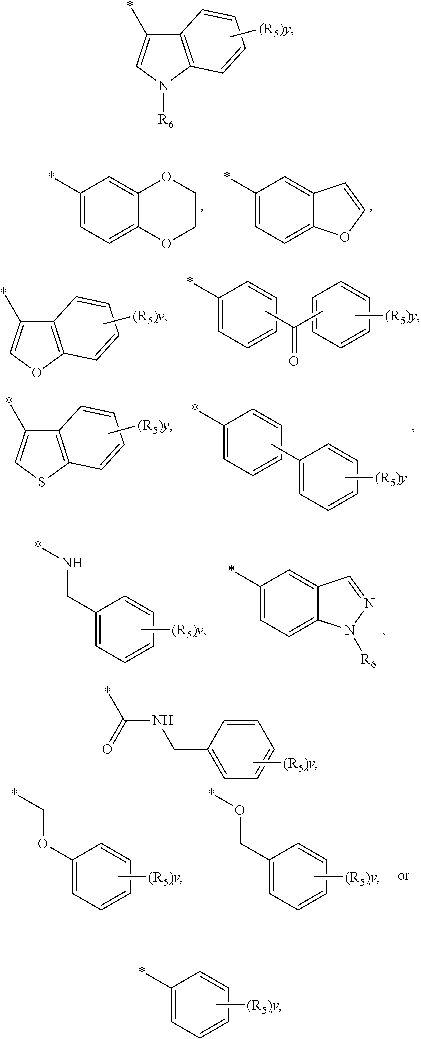

B is (CH₂)₃ Or CH(CH₃)(CH₂)₃,

each R₁ is chloro, bromo, nitro, or trifluoromethyl,

x is 1 or 2,

y is 0,

R₂ is hydrogen,

R₃ and R₄ are independently methyl or ethyl,

and tautomers or pharmaceutically acceptable salts thereof.

In some embodiments, the invention is a compound having the formula I, which is:

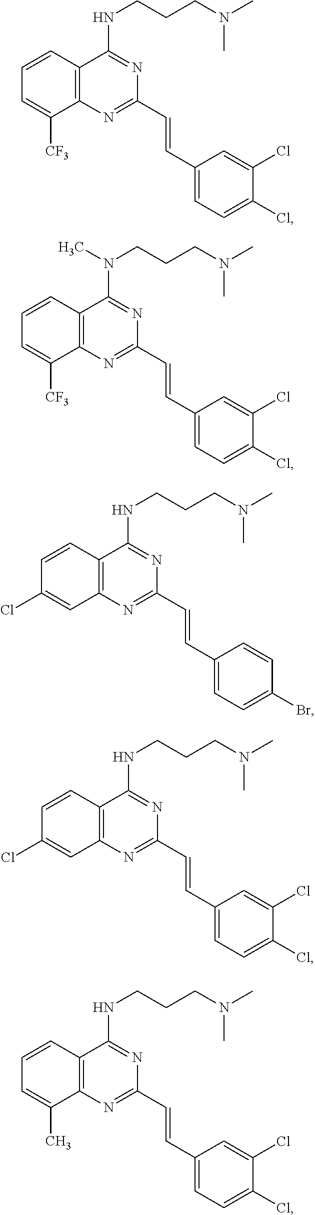

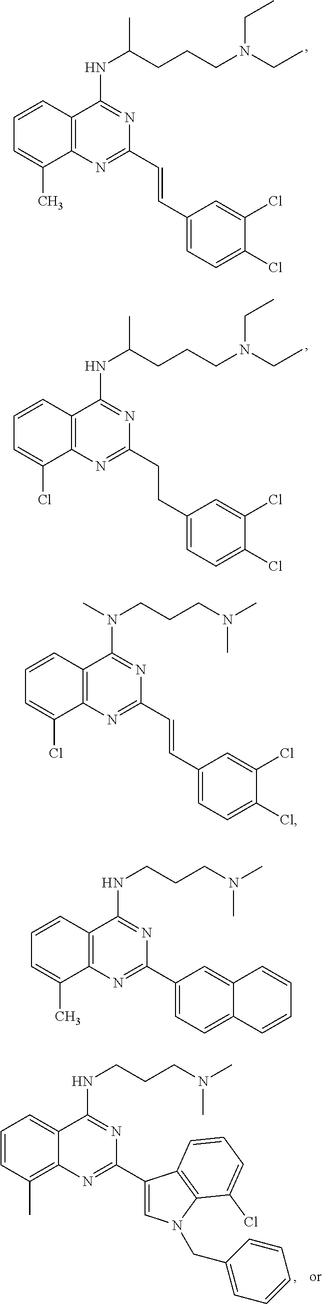

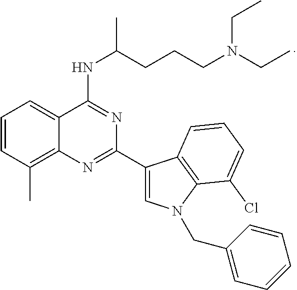 [SMILES: CCN(CC)CCCC(C)Nc1nc(-c2cn(Cc3ccccc3)c3c(Cl)cccc23)nc2c(C)cccc12]

In some embodiments, the invention is a compound selected from:

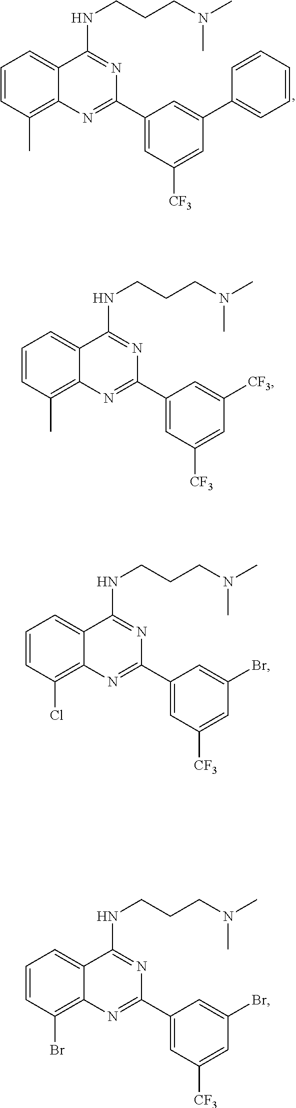 [SMILES: Cc1cccc2c(NCCCN(C)C)nc(-c3cc(-c4ccccc4)cc(C(F)(F)F)c3)nc12 ; Cc1cccc2c(NCCCN(C)C)nc(-c3cc(C(F)(F)F)cc(C(F)(F)F)c3)nc12 ; CN(C)CCCNc1nc(-c2cc(Br)cc(C(F)(F)F)c2)nc2c(Cl)cccc12 ; CN(C)CCCNc1nc(-c2cc(Br)cc(C(F)(F)F)c2)nc2c(Br)cccc12]

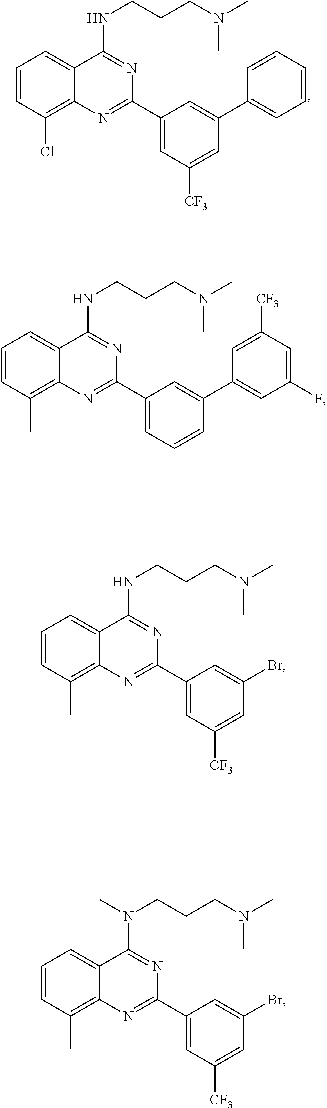 [SMILES: CN(C)CCCNc1nc(-c2cc(-c3ccccc3)cc(C(F)(F)F)c2)nc2c(Cl)cccc12 ; Cc1cccc2c(NCCCN(C)C)nc(-c3cccc(-c4cc(F)cc(C(F)(F)F)c4)c3)nc12 ; Cc1cccc2c(NCCCN(C)C)nc(-c3cc(Br)cc(C(F)(F)F)c3)nc12 ; Cc1cccc2c(N(C)CCCN(C)C)nc(-c3cc(Br)cc(C(F)(F)F)c3)nc12]

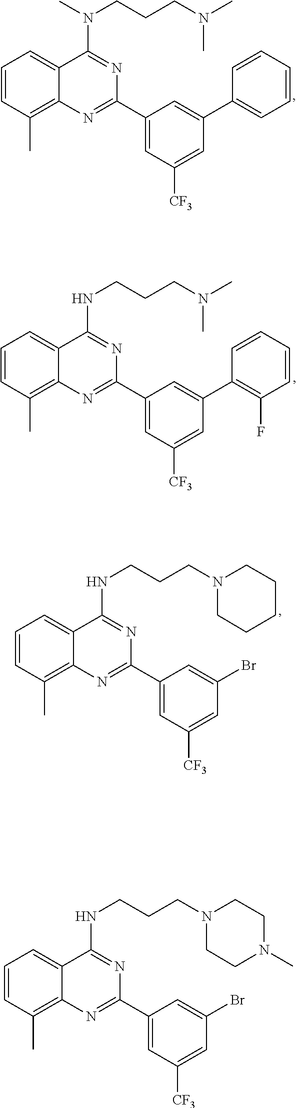 [SMILES: Cc1cccc2c(N(C)CCCN(C)C)nc(-c3cc(-c4ccccc4)cc(C(F)(F)F)c3)nc12 ; Cc1cccc2c(NCCCN(C)C)nc(-c3cc(-c4ccccc4F)cc(C(F)(F)F)c3)nc12 ; Cc1cccc2c(NCCCN3CCCCC3)nc(-c3cc(Br)cc(C(F)(F)F)c3)nc12 ; Cc1cccc2c(NCCCN3CCN(C)CC3)nc(-c3cc(Br)cc(C(F)(F)F)c3)nc12]

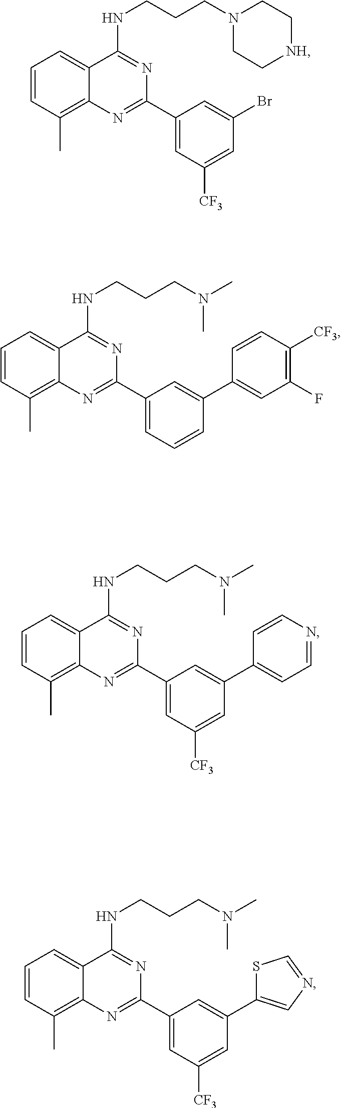 [SMILES: Cc1cccc2c(NCCCN3CCNCC3)nc(-c3cc(Br)cc(C(F)(F)F)c3)nc12 ; Cc1cccc2c(NCCCN(C)C)nc(-c3cccc(-c4ccc(C(F)(F)F)c(F)c4)c3)nc12 ; Cc1cccc2c(NCCCN(C)C)nc(-c3cc(-c4ccncc4)cc(C(F)(F)F)c3)nc12 ; Cc1cccc2c(NCCCN(C)C)nc(-c3cc(-c4cncs4)cc(C(F)(F)F)c3)nc12]

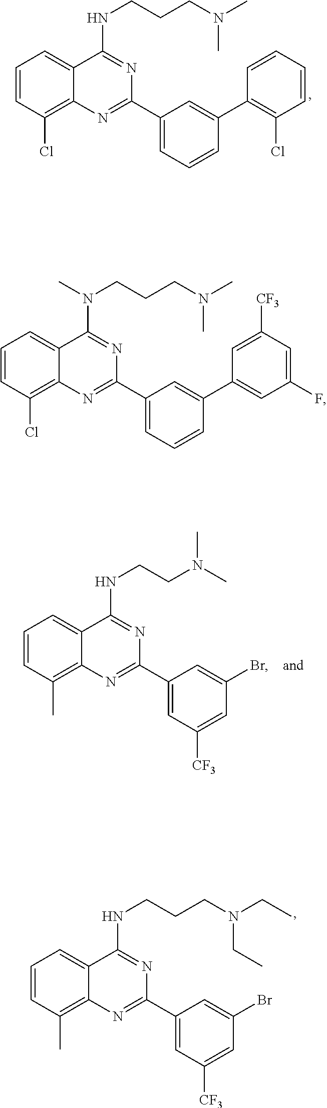 [SMILES: CN(C)CCCNc1nc(-c2cccc(-c3ccccc3Cl)c2)nc2c(Cl)cccc12 ; CN(C)CCCN(C)c1nc(-c2cccc(-c3cc(F)cc(C(F)(F)F)c3)c2)nc2c(Cl)cccc12 ; Cc1cccc2c(NCCN(C)C)nc(-c3cc(Br)cc(C(F)(F)F)c3)nc12 ; CCN(CC)CCCNc1nc(-c2cc(Br)cc(C(F)(F)F)c2)nc2c(C)cccc12]

or a stereoisomer or a tautomer or a pharmaceutically acceptable salt thereof.

In one embodiment, the invention is a compound of formula II and tautomers or pharmaceutically acceptable salts thereof.

In some embodiment, the invention is a compound of formula II, wherein:

A is one of the following groups:

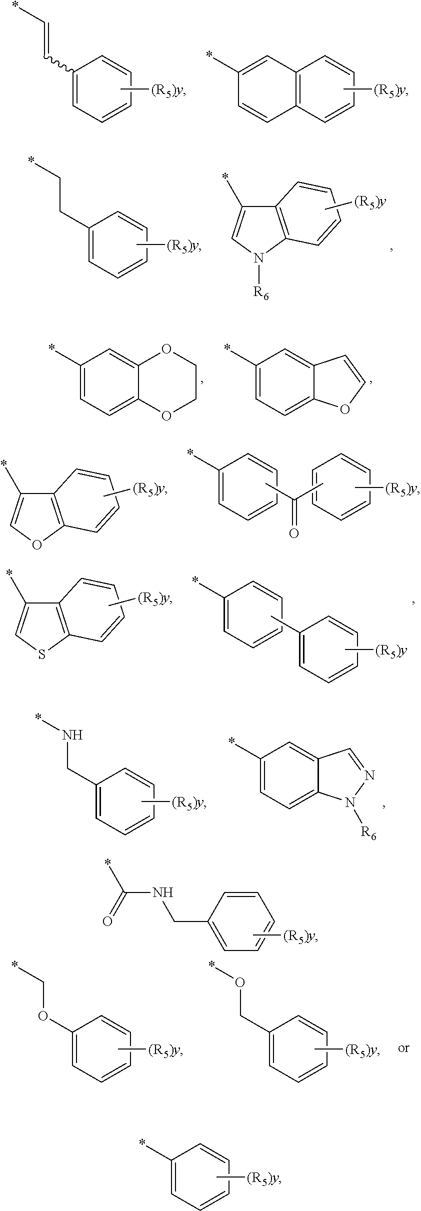

wherein * indicates the point of attachment of group A,

D is N or CH,

B is a methylene, ethylene, propylene, or butylene group, optionally substituted by one to four groups, which are independently C₁-C₄ alkyl, C₁-C₄ haloalkyl, C₁-C₄ alkoxy, or C₁-C₄ haloalkoxy,

each R₁ is independently halogen, C₁-C₄ alkyl, C₁-C₄ haloalkyl, nitro, or C₁-C₄-alkylamido,

each R₅ is independently halogen, C₁-C₄ alkyl, or C₁-C₄ haloalkyl,

R₂, R₃ and R₄ are independently hydrogen or optionally substituted C₁-C₄ alkyl, or C₁-C₄-alkyloxycarbonyl,

R₆ is hydrogen, optionally substituted C₁-C₄ alkyl, or C₁-C₄-alkyloxycarbonyl,

the wavy bond of formula A indicates that the double bond may be in a cis or trans configuration,

x is 1, 2, 3, or 4,

y is 1, 2, 3, 4, or 5,

or tautomers or pharmaceutically acceptable salts thereof.

In some embodiments, the invention is a compound of formula II, wherein:

A is one of the following groups:

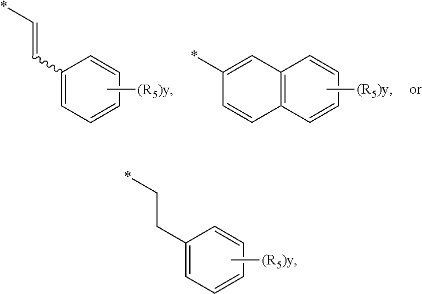

wherein * indicates the point of attachment of group A,

B is an ethylene, propylene, or butylene group, optionally substituted by one to four groups, which are independently C₁-C₃ alkyl, C₁-C₃ haloalkyl, C₁-C₃ alkoxy, or C₁-C₃ haloalkoxy,

D is N or CH,

each R₁ is independently halogen, C₁-C₃ alkyl, C₁-C₃ haloalkyl, nitro, or C₁-C₃-alkylamido,

each R₅ is independently halogen, C₁-C₃ alkyl, or C₁-C₃ haloalkyl,

R₂, R₃ and R₄ are independently hydrogen or optionally substituted C₁-C₃ alkyl, or C₁-C₃-alkyloxycarbonyl,

R₆ is hydrogen, optionally substituted C₁-C₃ alkyl, or C₁-C₃-alkyloxycarbonyl,

the wavy bond of formula A indicates that the double bond may be in a cis or trans configuration,

x is 1, 2, or 3,

y is 1, 2, or 3,

or tautomers or pharmaceutically acceptable salts thereof.

In some embodiments, the invention is a compound of formula II, wherein:

A is one of the following groups:

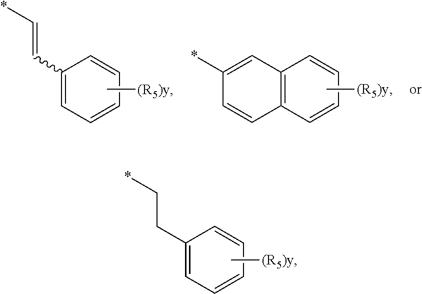

wherein * indicates the point of attachment of group A,

B is (CH₂)₂ or (CH₂)₃, optionally substituted by one to three C₁-C₆ alkyl groups, and

D is CH,

or tautomers or pharmaceutically acceptable salts thereof.

In some embodiments, the invention is a compound of formula II, wherein:

A is one of the following groups:

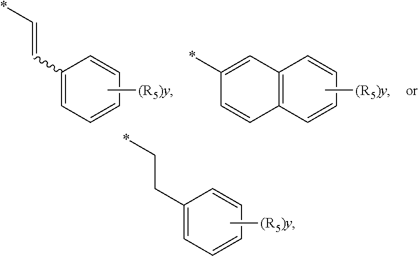

wherein * indicates the point of attachment of group A,

B is (CH₂)₂ or (CH₂)₃, optionally substituted by one or two C₁-C₃ alkyl groups,

each R₁ is chloro, fluoro, bromo, C₁-C₃ alkyl or C₁-C₃ haloalkyl,

x is 1 or 2,

each R₅ is halogen,

y is 1, 2 or 3,

R₂ is hydrogen,

R₃ and R₄ are independently C₁-C₃ alkyl,

or tautomers or pharmaceutically acceptable salts thereof

In some embodiments, invention is a compound of formula II, wherein:

A is one of the following groups:

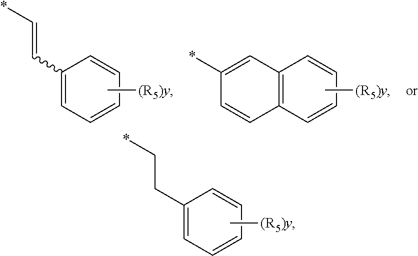

wherein * indicates the point of attachment of group A,

B is a (CH₂)₃ or (CH₂)₄ group, optionally substituted by one to four groups, which are independently methyl, ethyl, methyl or ethyl substituted by one to four chloro or fluoro groups, methoxy, ethoxy, methoxy or ethoxy substituted by one to four chloro or fluoro groups,

D is N or CH,

each R₁ is independently fluoro, chloro, bromo, methyl, ethyl, methyl or ethyl substituted by one to four chloro or fluoro groups, nitro, acetamido, propanamido,

each R₅ is independently fluoro, chloro, bromo, methyl, ethyl, methyl or ethyl substituted by one to four chloro or fluoro groups,

R₂, R₃ and R₄ are independently hydrogen, methyl, ethyl, methoxycarbonyl, or butoxycarbonyl,

R₆ is hydrogen, methyl, ethyl, benzyl, methoxycarbonyl, or butoxycarbonyl,

the double bond is in a trans configuration,

x is 1 or 2,

y is 1, 2, or 3,

or tautomers or pharmaceutically acceptable salts thereof.

In some embodiments, the invention is a compound of formula II, wherein:

A is one of the following groups:

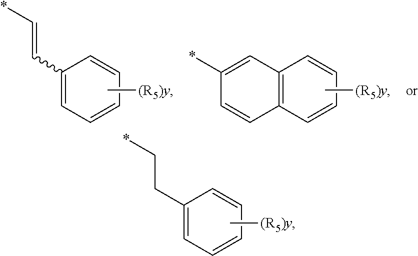

wherein * indicates the point of attachment of group A,

B is a (CH₂)₃ or (CH₂)₄ group, optionally substituted by one to four groups, which are independently methyl, ethyl, methyl or ethyl substituted by one to two chloro or fluoro groups, methoxy, ethoxy, methoxy or ethoxy substituted by one to two chloro or fluoro groups,

D is N or CH,

each R₁ is independently fluoro, chloro, bromo, methyl, ethyl, CH₂Cl, CHCl₂, CH₂F, CHF₂, CF₃, CH₂CF₃, CF₂CF₃, nitro, acetamido, propanamido,

each R₅ is independently fluoro, chloro, bromo, methyl, ethyl CH₂Cl, CHCl₂, CH₂F, CHF₂, CF₃, CH₂CF₃, or CF₂CF₃,

R₂, R₃ and R₄ are independently hydrogen, methyl, ethyl, or butoxycarbonyl,

R₆ is hydrogen, methyl, ethyl, benzyl, or butoxycarbonyl,

the double bond is in a trans configuration,

x is 1 or 2,

y is 1, 2, or 3,

or tautomers or pharmaceutically acceptable salts thereof.

In some embodiments, the invention is a compound of formula II, wherein:

A is one of the following groups:

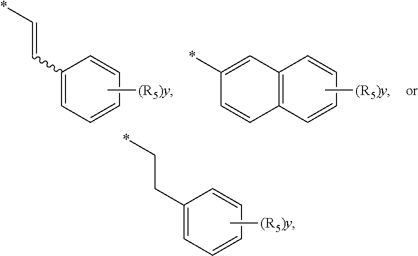

wherein * indicates the point of attachment of group A,

B is a (CH₂)₃ or (CH₂)₄ group, optionally substituted by one to four groups, which are independently methyl or ethyl,

D is CH,

each R₁ is independently fluoro, chloro, bromo, methyl, ethyl, CF₃, CF₂CF₃, nitro, or acetamido,

each R₅ is independently fluoro, chloro, bromo, methyl, ethyl, or CF₃,

R₂, R₃ and R₄ are independently hydrogen, methyl, ethyl, or butoxycarbonyl,

R₆ is hydrogen, methyl, ethyl, benzyl, or butoxycarbonyl,

the double bond is in a trans configuration,

x is 1 or 2,

y is 1, 2, or 3,

or tautomers or pharmaceutically acceptable salts thereof.

In some embodiments, the invention is a compound of formula II, wherein:

A is one of the following groups:

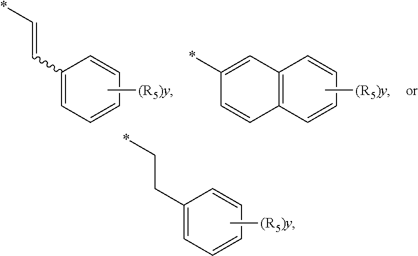

wherein * indicates the point of attachment of group A,

B is a (CH₂)₃ or (CH₂)₄ group, optionally substituted by one or two methyl or ethyl groups,

D is CH,

each R₁ is independently fluoro, chloro, bromo, CF₃, nitro, or acetamido,

each R₅ is independently fluoro, chloro, bromo, or CF₃,

R₂, R₃ and R₄ are independently hydrogen, methyl, ethyl, or butoxycarbonyl,

R₆ is hydrogen, methyl, ethyl, benzyl, or butoxycarbonyl,

the double bond is in a trans configuration,

x is 1 or 2,

y is 1, 2, or 3,

or tautomers or pharmaceutically acceptable salts thereof.

In some embodiments, the invention is a compound of formula II,

wherein A is one of the following groups:

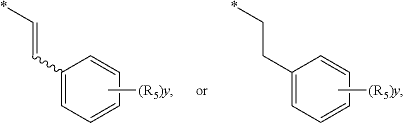

wherein * indicates the point of attachment of group A,

B is (CH₂)₂ or (CH₂)₃, substituted by one or two methyl or ethyl groups,

each R₁ is chloro, fluoro, methyl or trifluoromethyl,

x is 1 or 2,

each R₅ is fluoro or chloro,

y is 2 or 3,

R₂ is hydrogen,

R₃ and R₄ are independently methyl or ethyl,

R₆ and R₇ are hydrogen,

or tautomers or pharmaceutically acceptable salts thereof.

In some embodiments, the invention is a compound of formula II,

wherein A is one of the following groups:

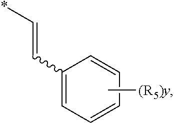

wherein * indicates the point of attachment of group A,

B is (CH₂)₃, substituted by one or two methyl groups,

each R₁ is chloro or trifluoromethyl,

x is 1 or 2,

each R₅ is independently fluoro or chloro,

y is 2,

R₂ is methyl,

R₃ and R₄ are independently methyl or ethyl,

or tautomers or pharmaceutically acceptable salts thereof.

In some embodiments, the invention is a compound of formula II,

wherein A is one of the following groups:

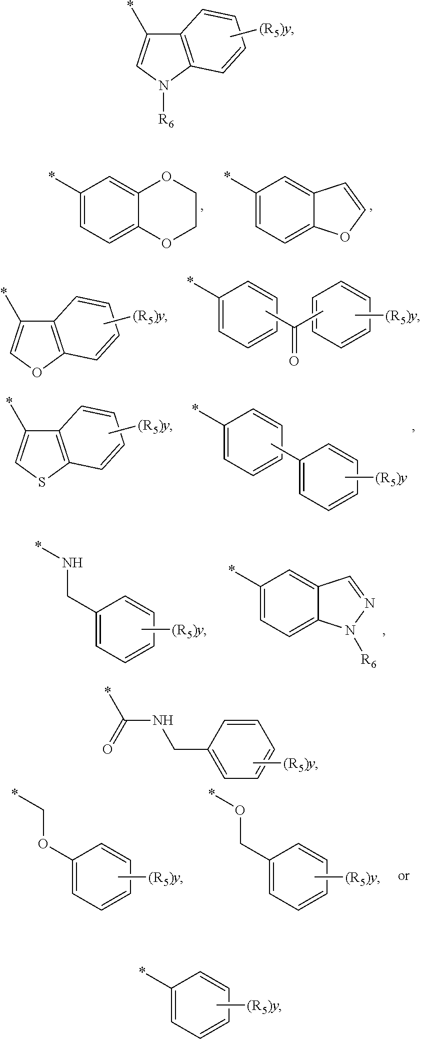

B is (CH₂)₃ Or (CH₂)₄, substituted by one or two methyl groups,

D is CH,

each R₁ is chloro, bromo, nitro, or trifluoromethyl,

x is 1 or 2,

each R₅ is independently fluoro or chloro,

R₂ is hydrogen or methyl,

R₃ and R₄ are independently hydrogen, methyl, or ethyl,

and tautomers or pharmaceutically acceptable salts thereof.

In some embodiments, the invention is a compound of formula II,

wherein A is one of the following groups:

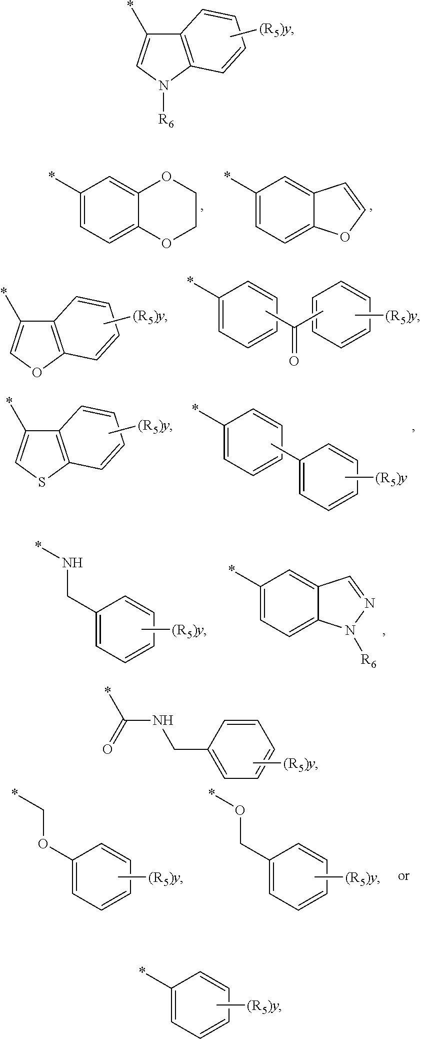

B is —COCH₂, —(CH₂)₃ or (CH₂)₄, substituted by one or two methyl groups,

D is CH,

each R₁ is chloro, bromo, nitro, or trifluoromethyl,

x is 1 or 2,

each R₅ is independently fluoro or chloro,

y is 2 or 3,

R₂ is hydrogen or methyl,

R₃ and R₄ are independently hydrogen, methyl, or ethyl,

and tautomers or pharmaceutically acceptable salts thereof.

In one embodiment, the invention is a compound of formula II, wherein A is one of the following groups:

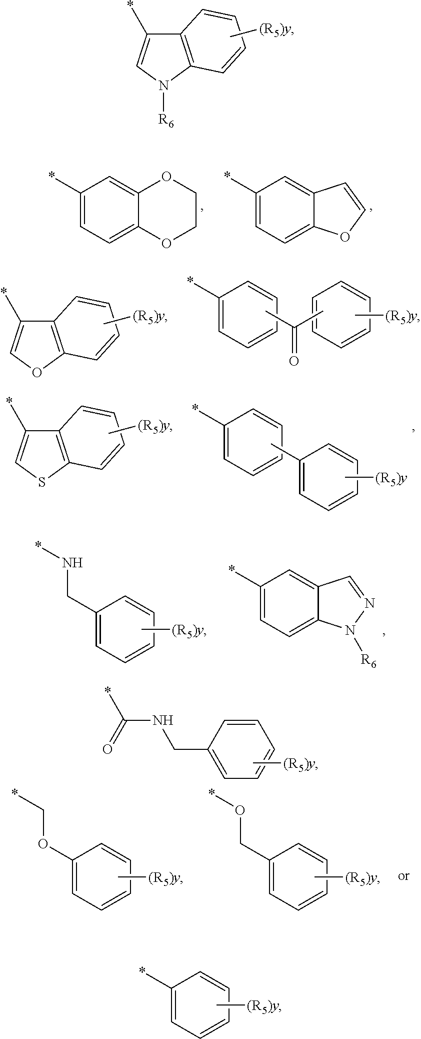

B is (CH₂)₃ Or CH(CH₃)(CH₂)₃,

D is N,

each R₁ is chloro, bromo, nitro, or trifluoromethyl,

x is 1 or 2,

y is 0,

R₂ is hydrogen or methyl,

R₃ and R₄ are independently hydrogen, methyl, or ethyl,

and tautomers or pharmaceutically acceptable salts thereof.

In one embodiment, the invention is a compound of formula II,

wherein A is one of the following groups:

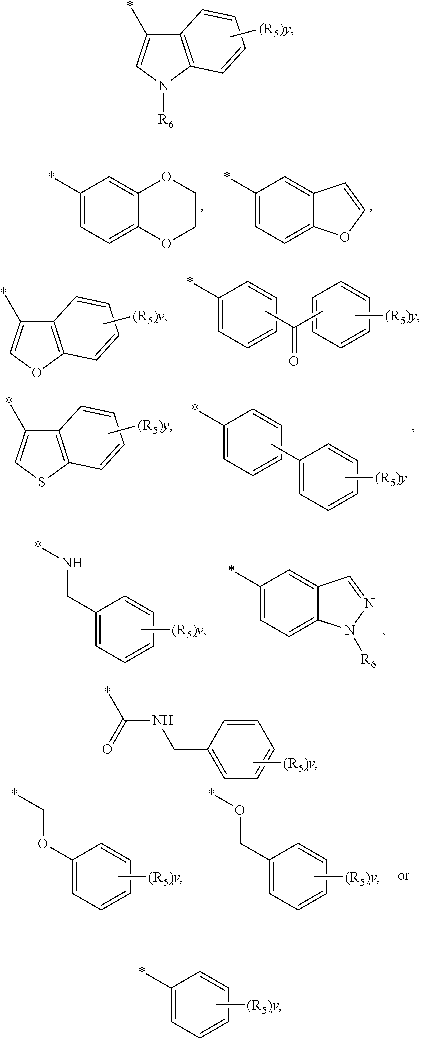

B is COCH₂—, (CH₂)₃ or CH(CH₃)(CH₂)₃,

D in N,

x is 1 or 2,

y is 0,

R₂ is hydrogen or methyl,

R₃ and R₄ are independently hydrogen, methyl, or ethyl,

and tautomers or pharmaceutically acceptable salts thereof.

In one embodiment, the invention is a compound of formula II,

wherein A is one of the following groups:

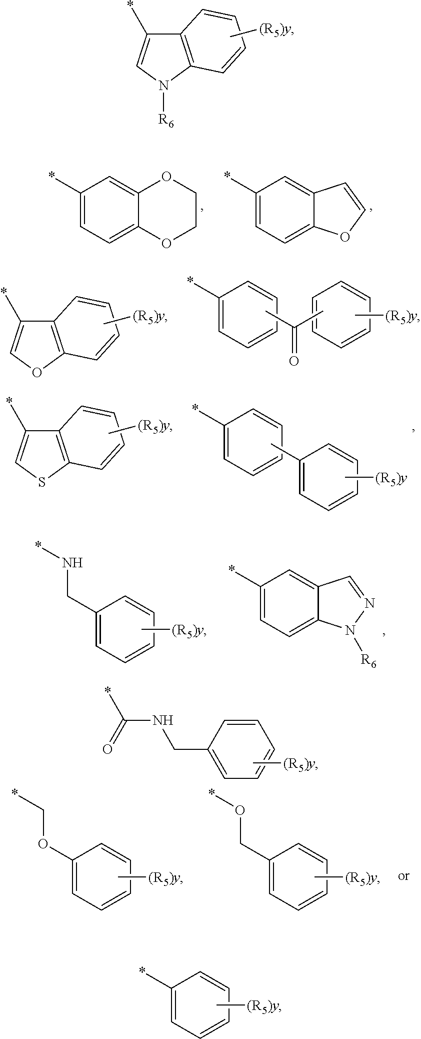

B is (CH₂)₃ Or CH(CH₃)(CH₂)₃,

each R₁ is chloro, bromo, nitro, or trifluoromethyl,

x is 1 or 2,

y is 0,

R₂ is hydrogen,

R₃ and R₄ are independently methyl or ethyl,

and tautomers or pharmaceutically acceptable salts thereof.

In one embodiment, the invention is a compound of formula II,

wherein A is one of the following groups:

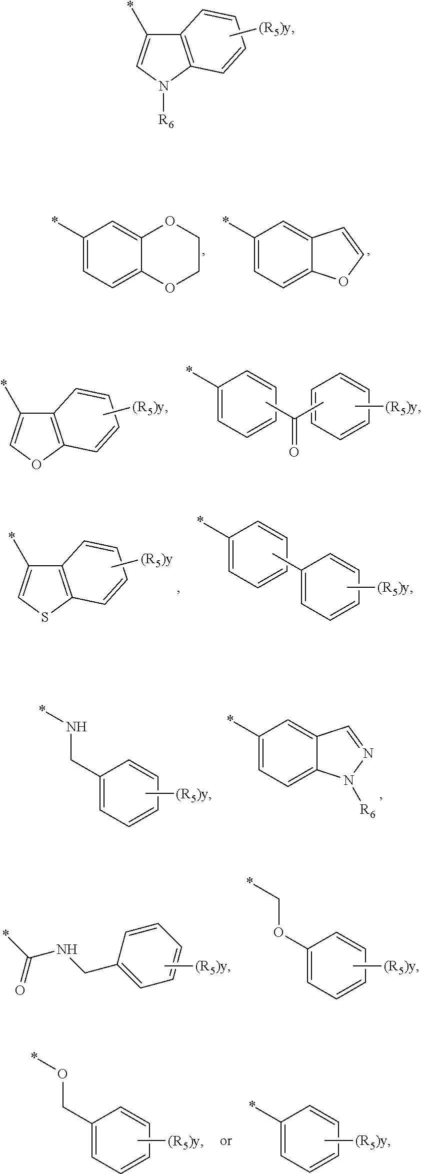

B is COCH₂—, COCH(CH₃)—, (CH₂)₃ or CH(CH₃)(CH₂)₃,

each R₁ is chloro, bromo, nitro, or trifluoromethyl,

x is 1 or 2,

y is 0,

R₂ is hydrogen,

R₃ and R₄ are independently methyl or ethyl,

and tautomers or pharmaceutically acceptable salts thereof.

In one embodiment, the invention is a compound of formula III and tautomers or pharmaceutically acceptable salts thereof.

In some embodiment, the invention is a compound of formula III, wherein:

A is one of the following groups:

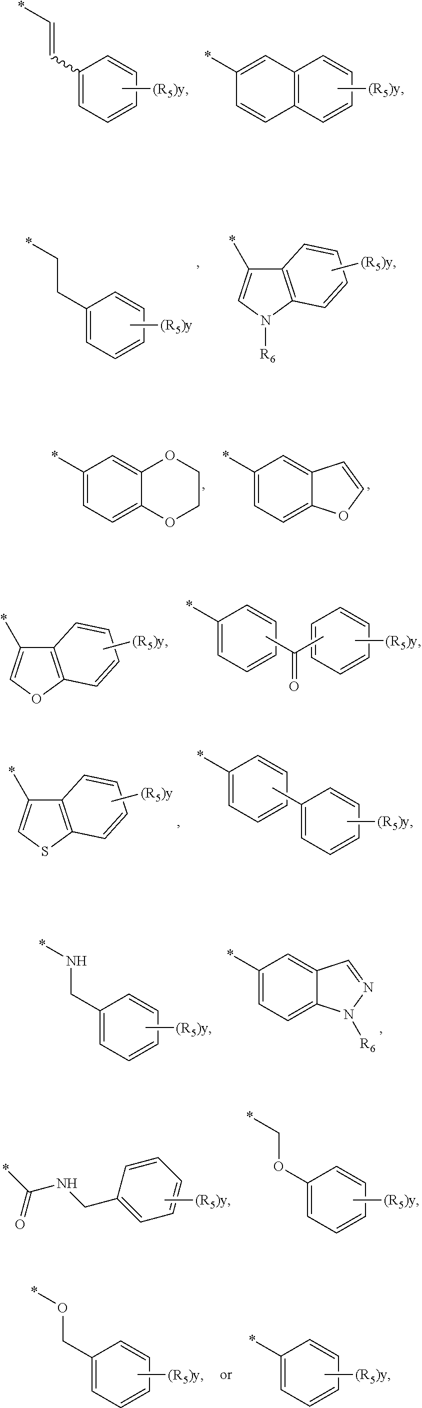

wherein H indicates the point of attachment of group A,

D is N or CH,

each R₁ is independently halogen, C₁-C₄ alkyl, C₁-C₄ haloalkyl, nitro, or C₁-C₄-alkylamido,

each R₅ is independently halogen, C₁-C₄ alkyl, or C₁-C₄ haloalkyl,

R₂, R₃ and R₄ are independently hydrogen or optionally substituted C₁-C₄ alkyl, or C₁-C₄-alkyloxycarbonyl,

R₆ is hydrogen, optionally substituted C₁-C₄ alkyl, or C₁-C₄-alkyloxycarbonyl,

the wavy bond of formula A indicates that the double bond may be in a cis or trans configuration,

x is 1, 2, 3, or 4,

y is 1, 2, 3, 4, or 5,

or tautomers or pharmaceutically acceptable salts thereof.

In some embodiments, the invention is a compound of formula III, wherein:

A is one of the following groups:

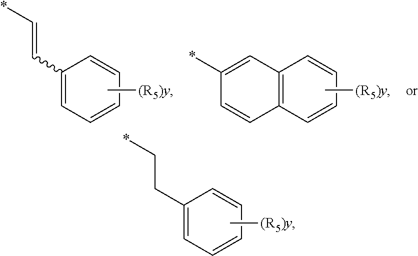

wherein * indicates the point of attachment of group A,

B is an ethylene, propylene, or butylene group, optionally substituted by one to four groups, which are independently C₁-C₃ alkyl, C₁-C₃ haloalkyl, C₁-C₃ alkoxy, or C₁-C₃ haloalkoxy,

each R₁ is independently halogen, C₁-C₃ alkyl, C₁-C₃ haloalkyl, nitro, or C₁-C₃-alkylamido,

each R₅ is independently halogen, C₁-C₃ alkyl, or C₁-C₃ haloalkyl,

R₂, R₃ and R₄ are independently hydrogen or optionally substituted C₁-C₃ alkyl, or C₁-C₃-alkyloxycarbonyl,

R₆ is hydrogen, optionally substituted C₁-C₃ alkyl, or C₁-C₃-alkyloxycarbonyl,

the wavy bond of formula A indicates that the double bond may be in a cis or trans configuration,

x is 1, 2, or 3,

y is 1, 2, or 3,

or tautomers or pharmaceutically acceptable salts thereof.

In some embodiments, the invention is a compound of formula III, wherein:

A is one of the following groups:

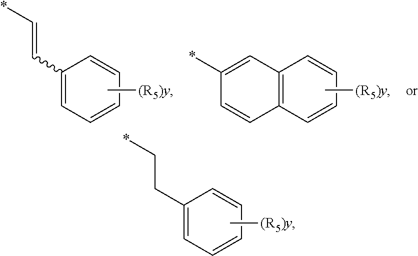

wherein * indicates the point of attachment of group A,

D is N or CH,

each R₁ is independently fluoro, chloro, bromo, methyl, ethyl, methyl or ethyl substituted by one to four chloro or fluoro groups, nitro, acetamido, propanamido,

each R₅ is independently fluoro, chloro, bromo, methyl, ethyl, methyl or ethyl substituted by one to four chloro or fluoro groups,

R₂, R₃ and R₄ are independently hydrogen, methyl, ethyl, methoxycarbonyl, or butoxycarbonyl,

R₆ is hydrogen, methyl, ethyl, benzyl, methoxycarbonyl, or butoxycarbonyl,

the double bond is in a trans configuration,

x is 1 or 2,

y is 1, 2, or 3,

or tautomers or pharmaceutically acceptable salts thereof.

In some embodiments, the invention is a compound of formula III, wherein:

A is one of the following groups:

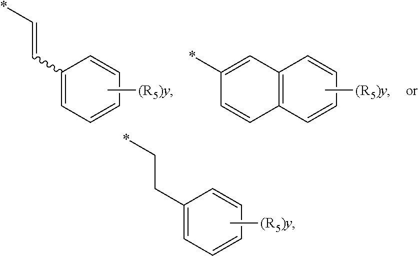

wherein * indicates the point of attachment of group A,

D is N or CH,

each R₁ is independently fluoro, chloro, bromo, methyl, ethyl, CH₂Cl, CHCl₂, CH₂F, CHF₂, CF₃, CH₂CF₃, CF₂CF₃, nitro, acetamido, propanamido,

each R₅ is independently fluoro, chloro, bromo, methyl, ethyl CH₂Cl, CHCl₂, CH₂F, CHF₂, CF₃, CH₂CF₃, or CF₂CF₃,

R₂, R₃ and R₄ are independently hydrogen, methyl, ethyl, or butoxycarbonyl,

R₆ is hydrogen, methyl, ethyl, benzyl, or butoxycarbonyl,

the double bond is in a trans configuration,

x is 1 or 2,

y is 1, 2, or 3,

or tautomers or pharmaceutically acceptable salts thereof.

In some embodiments, the invention is a compound of formula III, wherein:

A is one of the following groups:

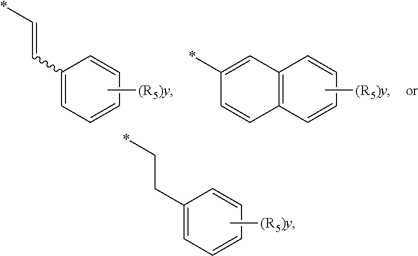

wherein * indicates the point of attachment of group A,

D is N or CH,

each R₁ is independently fluoro, chloro, bromo, methyl, ethyl, CF₃, CF₂CF₃, nitro, or acetamido,

each R₅ is independently fluoro, chloro, bromo, methyl, ethyl, or CF₃,

R₂, R₃ and R₄ are independently hydrogen, methyl, ethyl, or butoxycarbonyl,

R₆ is hydrogen, methyl, ethyl, benzyl, or butoxycarbonyl,

the double bond is in a trans configuration,

x is 1 or 2,

y is 1, 2, or 3,

or tautomers or pharmaceutically acceptable salts thereof.

In some embodiments, the invention is a compound of formula III, wherein:

A is one of the following groups:

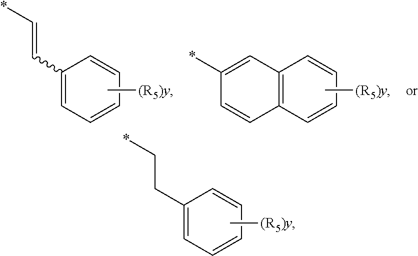

wherein * indicates the point of attachment of group A,

D is N or CH,

each R₁ is independently fluoro, chloro, bromo, CF₃, nitro, or acetamido,

each R₅ is independently fluoro, chloro, bromo, or CF₃,

R₂, R₃ and R₄ are independently hydrogen, methyl, ethyl, or butoxycarbonyl,

R₆ is hydrogen, methyl, ethyl, benzyl, or butoxycarbonyl,

the double bond is in a trans configuration,

x is 1 or 2,

y is 1, 2, or 3,

or tautomers or pharmaceutically acceptable salts thereof.

In some embodiments, the invention is a compound of formula III,

wherein A is one of the following groups:

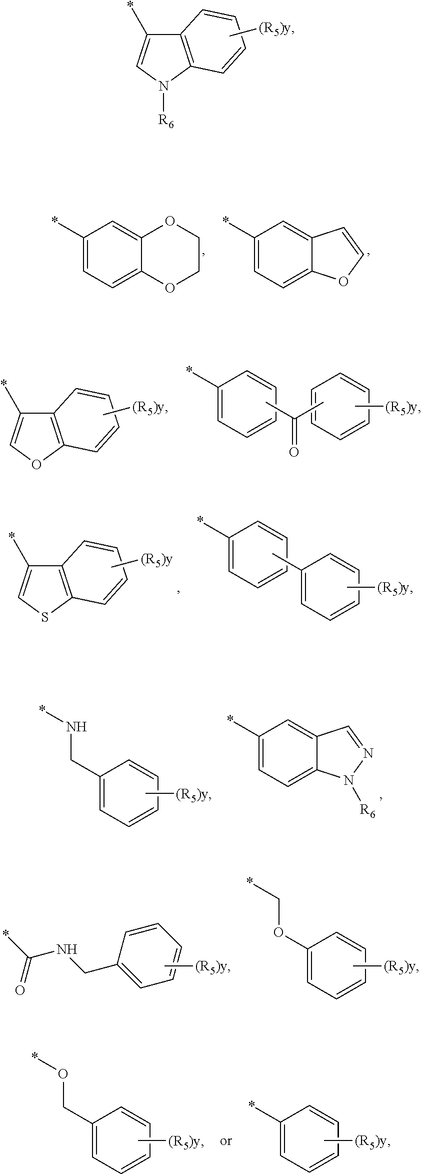

D is CH,

each R₁ is chloro, bromo, nitro, or trifluoromethyl,

x is 1 or 2,

each R₅ is independently fluoro or chloro,

y is 2 or 3,

R₂ is hydrogen or methyl,

R₃ and R₄ are independently hydrogen, methyl, or ethyl,

and tautomers or pharmaceutically acceptable salts thereof.

In one embodiment, the invention is a compound of formula III,

wherein A is one of the following groups:

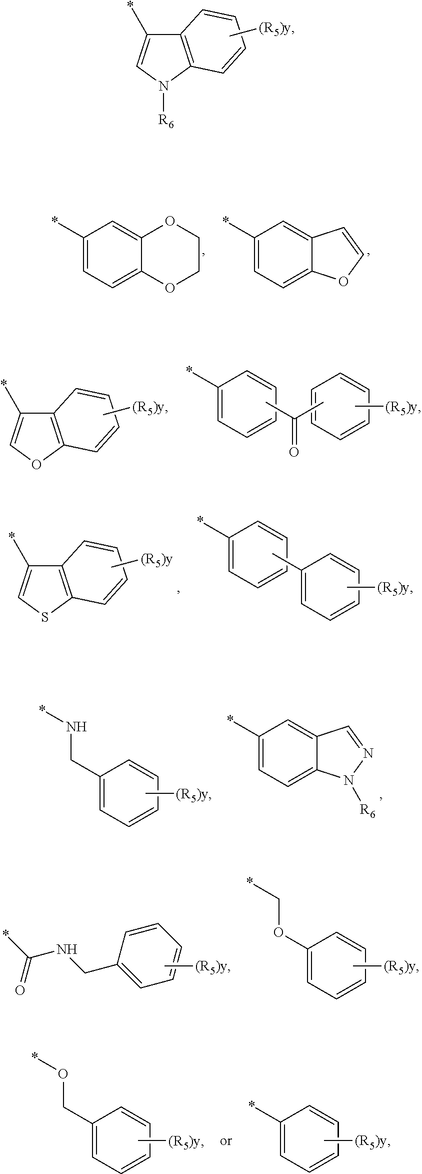

D is N,

each R₁ is chloro, bromo, nitro, or trifluoromethyl,

x is 1 or 2,

y is 0,

R₂ is hydrogen or methyl,

R₃ and R₄ are independently hydrogen, methyl, or ethyl,

and tautomers or pharmaceutically acceptable salts thereof.

In one embodiment, the invention is a compound of formula III,

wherein A is one of the following groups:

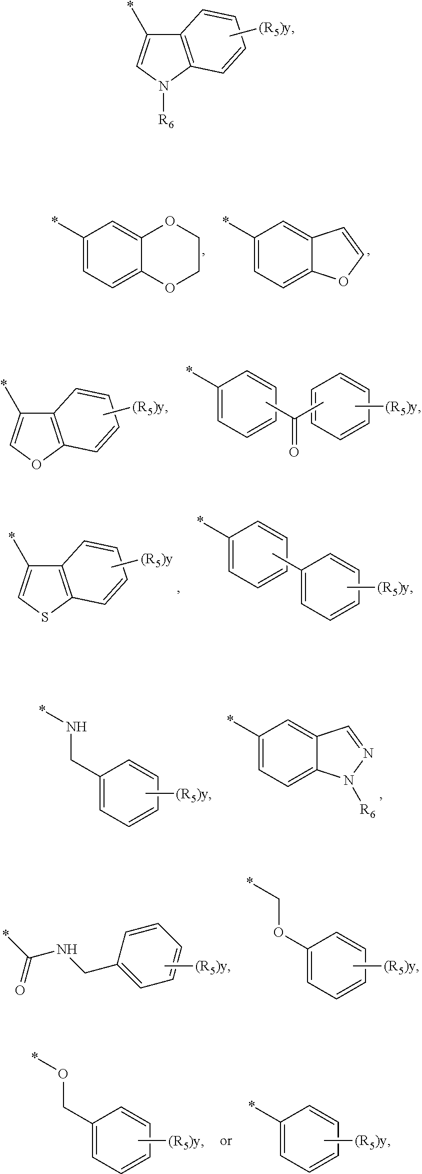

D is CH,

each R₁ is chloro, bromo, nitro, or trifluoromethyl,

x is 1 or 2,

y is 0,

R₂ is hydrogen,

R₃ and R₄ are independently methyl or ethyl,

and tautomers or pharmaceutically acceptable salts thereof.

In some embodiments, the invention is a compound having the formula II or III, which is:

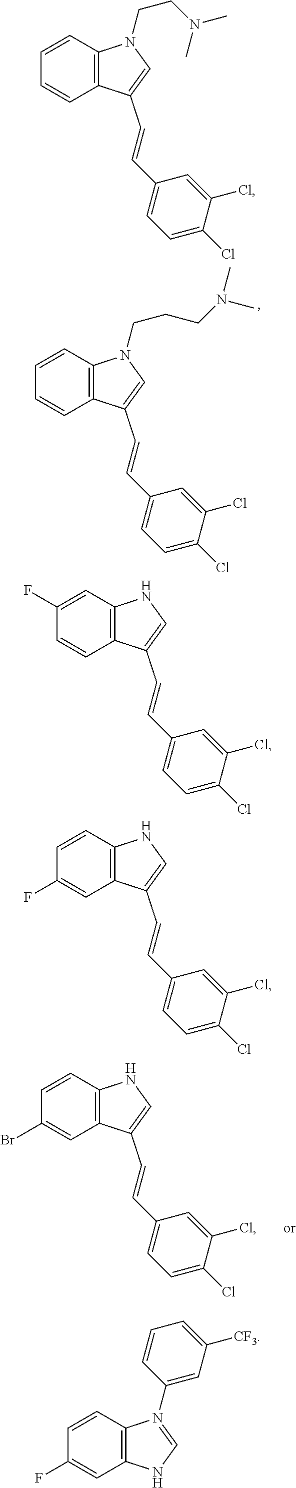

In some embodiments, the invention is a compound selected from

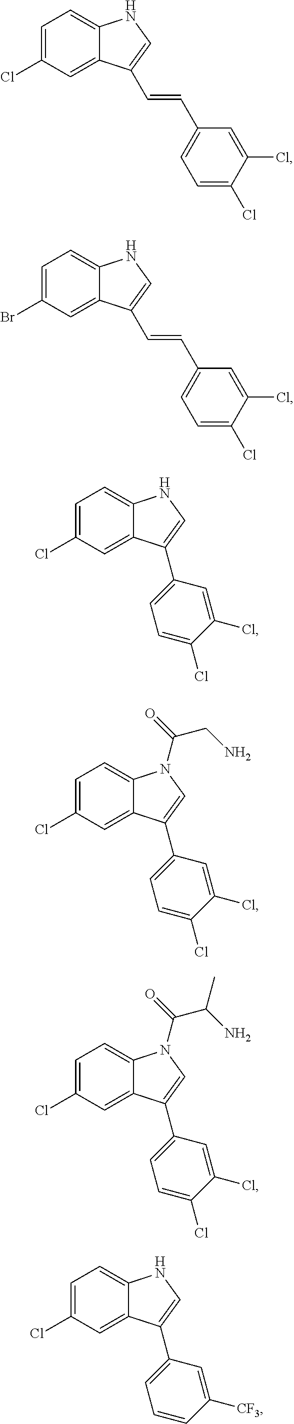

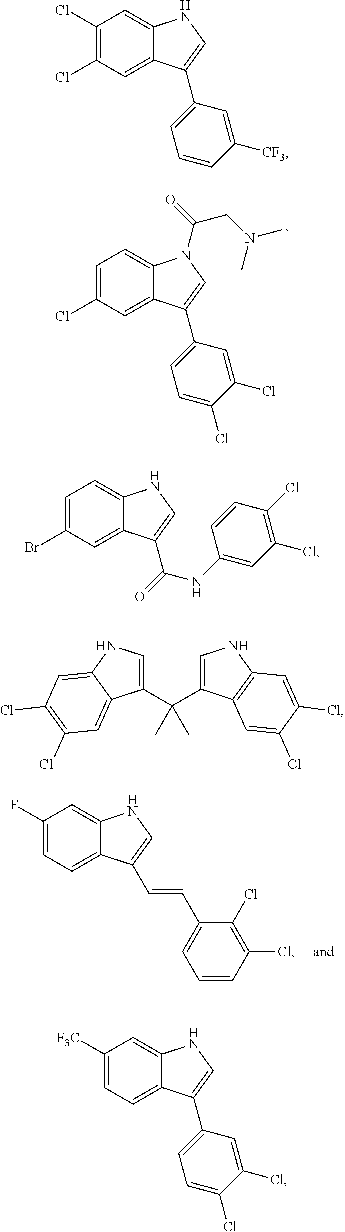

or a tautomer or a stereoisomer or a pharmaceutically acceptable salt thereof.

In some embodiments, the invention is a compound of formula Ia, wherein B is CH(CH₃)(CH₂)₃, and L, Ar, R₁, R₂, R₃, and R₄, are as indicated in Table 1.

In some embodiments, the invention is a compound of formula I, wherein A is

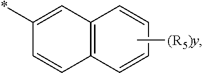

B is (CH₂)₃, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula I, wherein A is

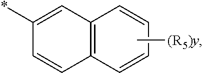

B is CH(CH₃)(CH₂)₃, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula I, wherein A is

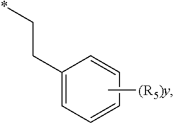

B is (CH₂)₃, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula I, wherein A is

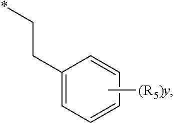

B is CH(CH₃)(CH₂)₃, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula II,

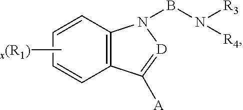

where A is

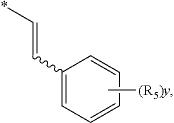

the double bond is in the trans configuration, B is (CH₂)₃, D is CH, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula II, wherein A is

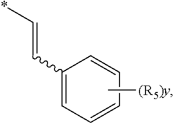

the double bond is in the trans configuration, B is CH(CH₃)(CH₂)₃, D is CH, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments the invention is a compound of formula II, wherein A is

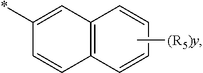

B is (CH₂)₃, D is CH, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula II, wherein A is

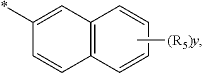

B is CH(CH₃)(CH₂)₃, D is CH, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula II, wherein A is

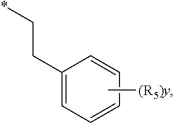

B is (CH₂)₃, D is CH, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula II, wherein A is

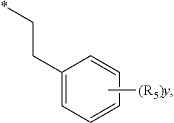

B is CH(CH₃)(CH₂)₃, D is CH, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula II, wherein A is

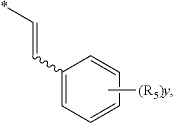

the double bond is in the trans configuration, B is (CH₂)₃, D is N, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula II, wherein A is

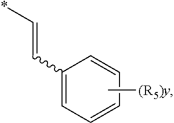

the double bond is in the trans configuration, B is CH(CH₃)(CH₂)₃, D is N, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula II, wherein A is

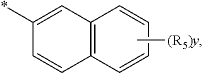

B is (CH₂)₃, D is N, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula II, wherein A is

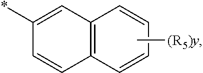

B is CH(CH₃)(CH₂)₃, D is N, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula II, wherein A is

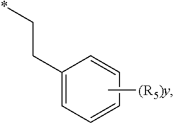

B is (CH₂)₃, D is N, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula II, wherein A is

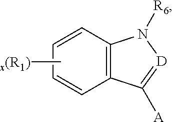

B is CH(CH₃)(CH₂)₃, D is N, and R₁, R₂, R₃, and R₄, are as indicated in Table 1. In further embodiments, R₅, is 3,4-dichloro; 3,5-dichloro; 3,4-difluoro; or 2,4,6-trichloro.

In some embodiments, the invention is a compound of formula III,

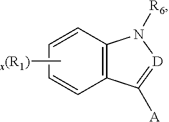

wherein A is

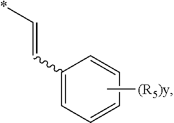

the double bond is in the trans configuration, D is CH, R₆ is H, and R₁ is as indicated in Table 1. In further embodiments, R₅, is 3,4-dichloro; 3,5-dichloro; 3,4-difluoro; or 2,4,6-trichloro.

In some embodiments, the invention is a compound of formula III, wherein A is

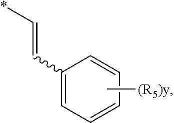

the double bond is in the trans configuration, D is CH, R₆ is butoxycarbonyl, and R₁ is as indicated in Table 1. In further embodiments, R₅, is 3,4-dichloro; 3,5-dichloro; 3,4-difluoro; or 2,4,6-trichloro.

In some embodiments, the invention is a compound of formula III, wherein A is

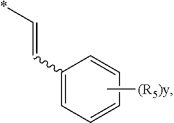

the double bond is in the trans configuration, D is CH, R₆ is benzyl, and R₁ is as indicated in Table 1. In further embodiments, R₅, is 3,4-dichloro; 3,5-dichloro; 3,4-difluoro; or 2,4,6-trichloro.

In some embodiments, the invention is a compound of formula III, wherein A is

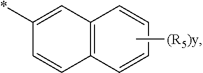

D is CH, R₆ is H, and R₁ is as indicated in Table 1. In further embodiments, R₅, is 3,4-dichloro; 3,5-dichloro; 3,4-difluoro; or 2,4,6-trichloro.

In some embodiments, the invention is a compound of formula III, wherein A is

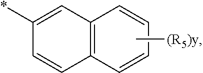

D is CH, R₆ is butoxycarbonyl, and R₁ is as indicated in Table 1. In further embodiments, R₅, is 3,4-dichloro; 3,5-dichloro; 3,4-difluoro; or 2,4,6-trichloro.

In some embodiments, the invention is a compound of formula III, wherein A is

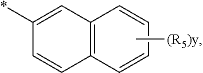

D is CH, R₆ is benzyl, and R₁ is as indicated in Table 1. In further embodiments, R₅, is 3,4-dichloro; 3,5-dichloro; 3,4-difluoro; or 2,4,6-trichloro.

In some embodiments, the invention is a compound of formula III, wherein A is

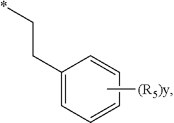

D is CH, R₆ is H, and R₁ is as indicated in Table 1. In further embodiments, R₅, is 3,4-dichloro; 3,5-dichloro; 3,4-difluoro; or 2,4,6-trichloro.

In some embodiments, the invention is a compound of formula III, wherein A is

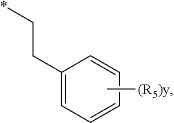

D is CH, R₆ is butoxycarbonyl, and R₁ is as indicated in Table 1. In further embodiments, R₅, is 3,4-dichloro; 3,5-dichloro; 3,4-difluoro; or 2,4,6-trichloro 1.

In some embodiments, the invention is a compound of formula III, wherein A is

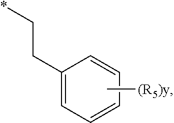

D is CH, R₆ is benzyl, and R₁ is as indicated in Table 1. In further embodiments, R₅, is 3,4-dichloro; 3,5-dichloro; 3,4-difluoro; or 2,4,6-trichloro.

The isostere concept was formulated by Irving Langmuir in 1919, and later modified by Grimm. Hans Erlenmeyer extended the concept to biological systems in 1932. Classical isosteres are defined as being atoms, ions and molecules that had identical outer shells of electrons, This definition has now been broadened to include groups that produce compounds that can sometimes have similar biological activities. Some evidence for the validity of this notion was the observation that some pairs, such as benzene and thiophene, thiophene and furan, and even benzene and pyridine, exhibited similarities in many physical and chemical properties.

A biologically-active compound containing an isostere is called a bioisostere. This is frequently used in drug design: the bioisostere will still be recognized and accepted by the body, but its functions there will be altered as compared to the parent molecule.

One of skill in the art will readily recognize that simple isosteres of the compounds disclosed herein, wherein a carbon is replaced with a nitrogen or vice versa, can also be prepared readily through methods known to a skilled artisan. Such isosteres are part of the disclosed invention herein.

### Salts, Solvates, Tautomers and Radioisotopes

Compounds of the invention encompass all the salts of the disclosed compounds of Formulae I-III. Compounds of the invention also encompass all the salts of the disclosed compounds of Formulae Ia-IIIa. The present invention preferably includes all non-toxic pharmaceutically acceptable salts thereof of the disclosed compounds. Examples of pharmaceutically acceptable addition salts include inorganic and organic acid addition salts and basic salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like.

Acid addition salts can be formed by mixing a solution of the particular compound of the present invention with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, or the like. Basic salts can be formed by mixing a solution of the compound of the present invention with a solution of a pharmaceutically acceptable non-toxic base such as sodium hydroxide, potassium hydroxide, choline hydroxide, sodium carbonate and the like.

Compounds of the invention also encompass solvates of any of the disclosed compounds of Formulae I-III. Compounds of the invention also encompass solvates of any of the disclosed compounds of Formulae Ia-IIIa. Solvates typically do not significantly alter the physiological activity or toxicity of the compounds, and as such may function as pharmacological equivalents. The term “solvate” as used herein is a combination, physical association and/or solvation of a compound of the present invention with a solvent molecule such as, e.g. a disolvate, monosolvate or hemisolvate, where the ratio of solvent molecule to compound of the present invention is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. compounds of the invention may be present as solvated forms with a pharmaceutically acceptable solvent, such as water, methanol, ethanol, and the like, and it is intended that the invention includes both solvated and unsolvated forms of compounds of any of Formulae I-III and Ia-IIIa. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1) Article 12 (2004), and A. L. Bingham et al, Chem. Commun.: 603-604 (2001). A typical, non-limiting, process of preparing a solvate would involve dissolving a compound of any of Formulae I-III and Ia-IIIa in a desired solvent (organic, water, or a mixture thereof) at temperatures above about 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

Compounds of the invention also encompass tautomers of any of the disclosed compounds of Formulae I-III. Compounds of the invention also encompass tautomers of any of the disclosed compounds of Formulae Ia-IIIa. “Tautomers” are particular isomers of a compound in which a hydrogen and double bond have changed position with respect to the other atoms of the molecule. For a pair of tautomers to exist there must be a mechanism for interconversion. Examples of tautomers include keto-enol forms, imine-enamine forms, amide-imino alcohol forms, amidine-aminidine forms, nitroso-oxime forms, thio ketone-enethiol forms, N-nitroso-hydroxyazo forms, nitro-aci-nitro forms, and pyridione-hydroxypyridine forms.

Compounds of the invention can be isotopically-labeled (i.e., radio-labeled). Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively, and preferably ³H, ¹³C, and ¹⁴C.

Isotopically-labeled compounds of the invention can be prepared by methods known in the art in view of this disclosure. For example, tritiated compounds of the invention can be prepared by introducing tritium into the particular compound by catalytic dehalogenation with tritium. This method may include reacting a suitable halogen-substituted precursor of a compound of the invention with tritium gas in the presence of an appropriate catalyst such as Pd/C in the presence of a base. Other suitable methods for preparing tritiated compounds can be found in Filer, Isotopes in the Physical and Biomedical Sciences, Vol. 1, Labeled Compounds (Part A), Chapter 6 (1987). ¹⁴C-labeled compounds can be prepared by employing starting materials having a ¹⁴C carbon.

### Methods of Treatment

In some embodiments, the invention is a method of treating a microbial infection in a subject in need thereof, the method comprising or consisting essentially of administering a compound disclosed herein. In some embodiments, the compound is a compound of formula Ia, formula IIa, or formula IIIa. In some embodiments, the compound is a compound of formula I, formula II, or formula III. In some embodiments, the compound is a compound of formula I, formula II, formula III, formula Ia, formula IIa, or formula IIIa. In some embodiments, the compound is selected from Tables 1-15.

In some embodiments, the invention is a method of treating a microbial infection in a subject in need thereof, the method comprising administering a compound of formula Ia, formula IIa, or formula IIIa:

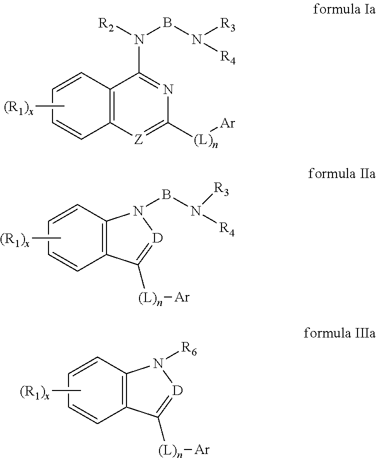

wherein
each L is independently selected from the group consisting of C₁-C₆ alkylene, C₂-C₆ alkenylene, C₂-C₆ alkynylene, —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —NR₇C(O)—, —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-,
n is 0 or 1,
Ar is aryl, optionally substituted with one to five R₅ groups; or heteroaryl, optionally substituted with one to four R₅ groups,
B is an alkylene group, optionally interrupted by an oxygen, a carbonyl (C═O), or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,
D is CR₇ or N,
each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl, or optionally substituted heteroaryl; or two R₁ groups are linked together to form an optionally substituted 5- or 6-membered aromatic moiety or an optionally substituted 4- to 7-membered non-aromatic cyclic moiety,
R₂ is hydrogen, optionally substituted C₁-C₆ alkyl, C(O)R₇, or C₁-C₆-alkyloxycarbonyl,
R₃ and R₄ are independently hydrogen, optionally substituted C₁-C₆ alkyl, optionally substituted aryl, C(O)R₇, or C₁-C₆-alkyloxycarbonyl; or R₃ and R₄, together with the nitrogen to which they are attached, form a 4- to 6-membered heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇,
each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), optionally substituted heterocyclyl, optionally substituted C₃-C₇ cycloalkyl, or optionally substituted heteroaryl; and/or two R₅ groups are linked together to form an optionally substituted 5- or 6-membered aromatic moiety or an optionally substituted 4- to 7-membered non-aromatic cyclic moiety,
R₆ is hydrogen, optionally substituted C₁-C₆ alkyl, optionally substituted aryl-(C₁-C₆ alkylene), optionally substituted C₁-C₆-alkylcarbonyl, or C₁-C₆-alkyloxycarbonyl,
each R₇ is independently hydrogen, C₁-C₆ alkyl, or C₁-C₆ haloalkyl, and
each R₈ is independently C₁-C₆ alkyl or C₁-C₆ haloalkyl,
Z is CH or N,
x is 0, 1, 2, 3, or 4, and
if x is 0, Ar is not unsubstituted;

or a stereoisomer or a tautomer or a pharmaceutically acceptable salt thereof.

In some embodiments, the invention is a method of treating a microbial infection in a subject in need thereof, the method comprising administering an antimicrobial agent and a compound disclosed herein.

In some embodiments, the invention is a method of treating a microbial infection in a subject in need thereof, the method comprising administering an antimicrobial agent and a compound of formula Ia, formula IIa, or formula IIIa:

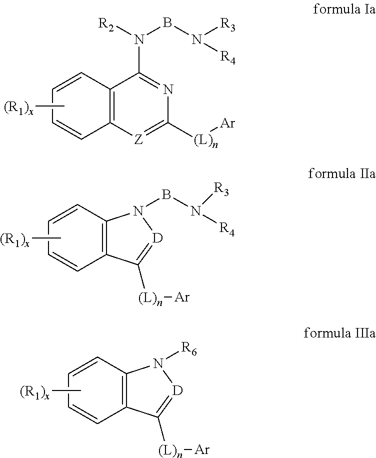

wherein,
each L is independently selected from the group consisting of C₁-C₆ alkylene, C₂-C₆ alkenylene, C₂-C₆ alkynylene, —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —NR₇C(O)—, —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-,
n is 0 or 1,
Ar is aryl, optionally substituted with one to five R₅ groups; or heteroaryl, optionally substituted with one to four R₅ groups,
B is an alkylene group, optionally interrupted by a carbonyl (C═O) or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,
D is CR₇ or N,
each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl, or optionally substituted heteroaryl; or two R₁ groups are linked together to form an optionally substituted 5- or 6-membered aromatic moiety or an optionally substituted 4- to 7-membered non-aromatic cyclic moiety,
R₂ is hydrogen, optionally substituted C₁-C₆ alkyl, C(O)R₇, or C₁-C₆-alkyloxycarbonyl,
R₃ and R₄ are independently hydrogen, optionally substituted C₁-C₆ alkyl, optionally substituted aryl, C(O)R₇, or C₁-C₆-alkyloxycarbonyl; or R₃ and R₄, together with the nitrogen to which they are attached, form a 4- to 6-membered heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇,
each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), optionally substituted heterocyclyl, or optionally substituted heteroaryl; and/or two R₅ groups are linked together to form an optionally substituted 5- or 6-membered aromatic moiety or an optionally substituted 4- to 7-membered non-aromatic cyclic moiety,
R₆ is hydrogen, optionally substituted C₁-C₆ alkyl, optionally substituted aryl-(C₁-C₆ alkylene), or C₁-C₆-alkyloxycarbonyl,
each R₇ is independently hydrogen, C₁-C₆ alkyl, or C₁-C₆ haloalkyl, and
each R₈ is independently C₁-C₆ alkyl or C₁-C₆ haloalkyl,
Z is CH or N,
x is 0, 1, 2, 3, or 4, and
if x is 0, Ar is not unsubstituted;
or a stereoisomer or a tautomer or a pharmaceutically acceptable salt thereof.

In some embodiments, the antimicrobial agent targets gram-negative bacteria. In some embodiments, the antimicrobial agent targets gram-positive bacteria. In some embodiments, the antimicrobial agent targets gram-negative and gram-positive bacteria. In some embodiments, the antimicrobial agent is a macrocyclic antibiotic, a quinolone antibiotic, a beta-lactam antibiotic, or an aminoglycoside antibiotic. In some embodiments, the antimicrobial agent is a macrocyclic antibiotic. In some embodiments, the macrocyclic antibiotic is a polymyxin antibiotic. In some embodiments, the polymyxin antibiotic is colistin. In some embodiments, the polymyxin antibiotic is polymyxin B. In some embodiments, the antimicrobial agent is a quinolone, fluoroquinolone, or beta-lactam antibiotic. In some embodiments, the antimicrobial agent is a quinolone antibiotic. Quinolone antibiotics include, but are not limited to, cinoxacin, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, sparfloxacin, temafloxacin, tosufloxacin, clinafloxacin, gatifloxacin, gemifloxacin, moxifloxacin, sitafloxacin, trovafloxacin, prulifloxacin, delafloxacin, and nemonoxacin. In some embodiments, the antimicrobial agent is a fluoroquinolone antibiotic. Fluoroquinolone antibiotics include, but are not limited to, ciprofloxacin, levofloxacin, moxifloxacin, lomifloxacin, norfloxacin, ofloxacin, sparfloxacin, and trovafloxacin. In some embodiments, the fluoroquinolone is ciprofloxacin. In some embodiments, the fluoroquinolone is levofloxacin. In some embodiments, the antimicrobial agent is a beta-lactam antibiotic. Beta-lactam antibiotics include, but are not limited to, penicillins, cephalosporins, cephamycins, and carbapenems. Exemplary beta-lactams include, but are not limited to, benzathine penicillin, penicillin G, penicillin V, procaine penicillin, cloxacillin, dicloxacillin, flucloxacillin, methicillin, nafcillin, oxacillin, temocillin, ampicillin, amoxicillin, epicillin, mecillinam, carbenicillin, ticarcillin, azlocillin, pivampicillin, hetacillin, bacampicillin, metampicillin, talampicillin, epicillin, mezlocillin, piperacillin, sulbencillin, faropenem, ertapenem, doripenem, imipenem, meropenem, biapenem, panipenem, cefazolin, cefalexin, cefadroxil, cefapirin, cefazedone, cefazaflur, cefradine, cefroxadine, deftezole, cefaloglycin, cefacetrile, cefalonium, cefaloridine, cefalotin, cefatrizine, cefaclor, cefotetan, cefoxitin, cefprozil, cefuroxime, cefuroxime axetil, cefamandole, cefminox, cefonicid, ceforanide, cefotiam, cefbuperazone, cefuzonam, cefmetazole, carbacephem, cefixime, ceftriaxone, ceftazidime, cefdinir, cefoperazone, cefcapene, cefdaloxime, ceftizoxime, cefmenoxime, cefotaxime, cefpiramide, cefpodoxime, ceftibuten, cefditoren, cefetamet, cefodizime, cefpimizole, cefsulodin, cefteram, ceftiolene, flomoxef, latamoxef, cefepime, cefozopran, cefpirome, cefquinome, ceftaroline fosamil, ceftolozane, ceftobiprole, ceftiofur, cefquinome, cefovecin, aztreonam, tigemonam, carumonam, nocardicin A, sulbactam, tazobactam, clavam, and avibactam. In some embodiments, the beta-lactam antibiotic is meropenem. In some embodiments, the antimicrobial agent is an aminoglycoside antibiotic.

Aminoglycoside antibiotics include, but are not limited to, kanamycin A, amikacin, tobramycin, dibekacin, gentamicin, sisomicin, netilmicin, neomycin B, neomycin C, neomycin E, and streptomycin. In some embodiments, the aminoglycoside is tobramycin.

In some embodiments, the invention is a method of treating a microbial infection in a subject in need thereof, the method comprising administering a quinolone antibiotic and a compound of formula Ia, formula IIa, or formula IIIa.

In some embodiments, the invention is a method of treating a microbial infection in a subject in need thereof, the method comprising administering a fluoroquinolone antibiotic and a compound of formula Ia, formula IIa, or formula IIIa.

In some embodiments, the invention is a method of treating a microbial infection in a subject in need thereof, the method comprising administering a beta-lactam antibiotic and a compound of formula Ia, formula IIa, or formula IIIa.

In some embodiments, the invention is a method of treating a microbial infection in a subject in need thereof, the method comprising administering an aminoglycoside antibiotic and a compound of formula Ia, formula IIa, or formula IIIa.

In some embodiments, the invention is a method of treating a microbial infection in a subject in need thereof, the method comprising administering a macrocyclic antibiotic and a compound of formula Ia, formula IIa, or formula IIIa. In some embodiments, the antimicrobial agent is a macrocyclic antibiotic. In one embodiment, the macrocyclic antibiotic is polymyxin. In a particular embodiment, the invention is a method of treating a microbial infection in a subject in need thereof, the method comprising administering a polymyxin antibiotic which is colistin and a compound of formula Ia, formula IIa, or formula IIIa.

In some embodiments, the invention is a method of treating a microbial infection in a subject in need thereof, the method comprising administering an antimicrobial agent and a compound of formula I, formula II, or formula III:

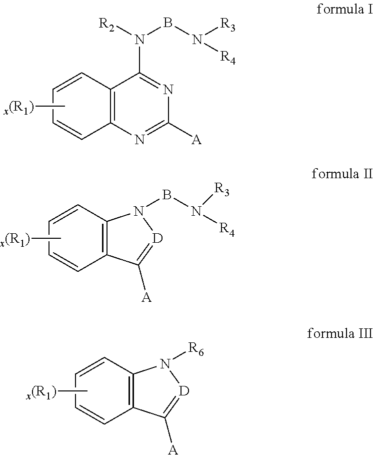

wherein

A is one of the following groups:

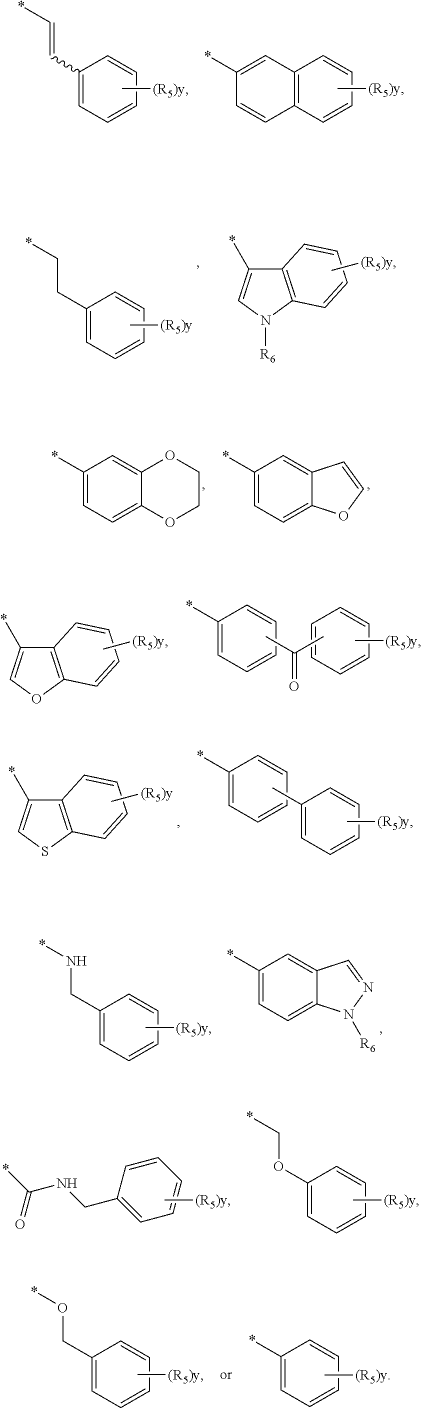

wherein * indicates the point of attachment of group A,

B is an alkylene group, optionally substituted by one to four groups, which are independently C₁-C₆ alkyl, C₁-C₆ haloalkyl, C₁-C₆ alkoxy, or C₁-C₆ haloalkoxy,

D is CH or a heteroatom,

each R₁ is independently halogen, C₁-C₆ alkyl, C₁-C₆ haloalkyl, nitro, or C₁-C₆-alkylamido,

each R₅ is independently halogen, C₁-C₆ alkyl, or C₁-C₆ haloalkyl,

R₂, R₃ and R₄ are independently hydrogen or optionally substituted C₁-C₆ alkyl, or C₁-C₆-alkyloxycarbonyl,

R₆ is hydrogen, optionally substituted C₁-C₆ alkyl, or C₁-C₆-alkyloxycarbonyl,

the wavy bond of formula A indicates that the double bond may be in a cis or trans configuration,

x is 0, 1, 2, 3, or 4,

y is 0, 1, 2, 3, 4, or 5, wherein both x and y are not simultaneously 0, and

and tautomers or pharmaceutically acceptable salts thereof.

In some embodiments, the invention is a method of treating a microbial infection in a subject in need thereof, the method comprising administering a macrocyclic antibiotic and a compound of formula I, formula II, or formula III. In some embodiments, the antimicrobial agent is a macrocyclic antibiotic, a quinolone antibiotic, a beta-lactam antibiotic, or an aminoglycoside antibiotic. In some embodiments, the antimicrobial agent is a macrocyclic antibiotic. In one embodiment, the macrocyclic antibiotic is polymyxin. In a particular embodiment, the invention is a method of treating a microbial infection in a subject in need thereof, the method comprising administering a polymyxin antibiotic which is colistin and a compound of formula I, formula II, or formula III.

In some embodiments, the invention is a method of treating a microbial infection in a subject in need thereof, the method comprising administering an antimicrobial agent and a compound of formula I or formula II:

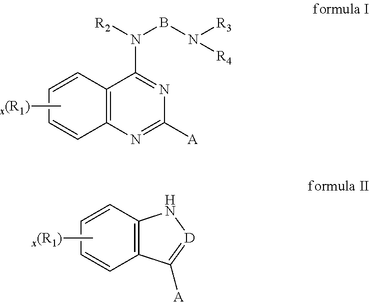

wherein,

A is one of the following groups:

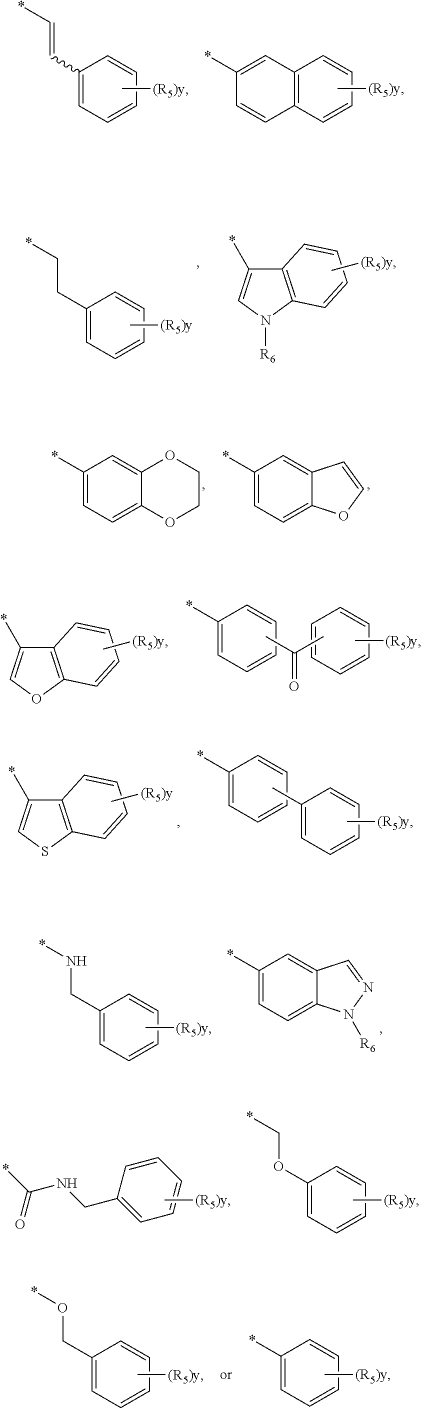

wherein * indicates the point of attachment of group A,

B is an alkylene group, optionally interrupted by a carbonyl (C═O) or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,

D is CH or a heteroatom,

each R₁ is independently halogen, C₁-C₆ alkyl, C₁-C₆ haloalkyl, nitro, or C₁-C₆-alkylamido,

each R₅ is independently halogen, C₁-C₆ alkyl, or C₁-C₆ haloalkyl,

R₂, R₃ and R₄ are independently hydrogen or optionally substituted C₁-C₆ alkyl, or C₁-C₆-alkyloxycarbonyl,

R₆ is hydrogen, optionally substituted C₁-C₆ alkyl, or C₁-C₆-alkyloxycarbonyl,

the wavy bond of formula A indicates that the double bond may be in a cis or trans configuration,

x is 0, 1, 2, 3, or 4,

y is 0, 1, 2, 3, 4, or 5, wherein both x and y are not simultaneously 0, and

or tautomers or pharmaceutically acceptable salts thereof.

In some embodiments, the antimicrobial agent is a macrocyclic antibiotic, a quinolone antibiotic, a beta-lactam antibiotic, or an aminoglycoside antibiotic. In some embodiments, the antimicrobial agent is a macrocyclic antibiotic. In one embodiment, the macrocyclic antibiotic is polymyxin. In a particular embodiment, the invention is a method of treating a microbial infection in a subject in need thereof, the method comprising administering a polymyxin antibiotic which is colistin and a compound of formula I or formula II. In some embodiments, the antimicrobial agent is a quinolone, fluoroquinolone, or beta-lactam antibiotic. In some embodiments, the antimicrobial agent is a quinolone antibiotic. In some embodiments, the antimicrobial agent is a fluoroquinolone antibiotic. In some embodiments, the antimicrobial agent is a beta-lactam antibiotic.

In particular embodiments, the invention is a method of treating a microbial infection in a subject in need thereof, the method comprising administering an antimicrobial agent and a compound of formula I or tautomers or pharmaceutically acceptable salts thereof to the subject, the compound of formula I being:

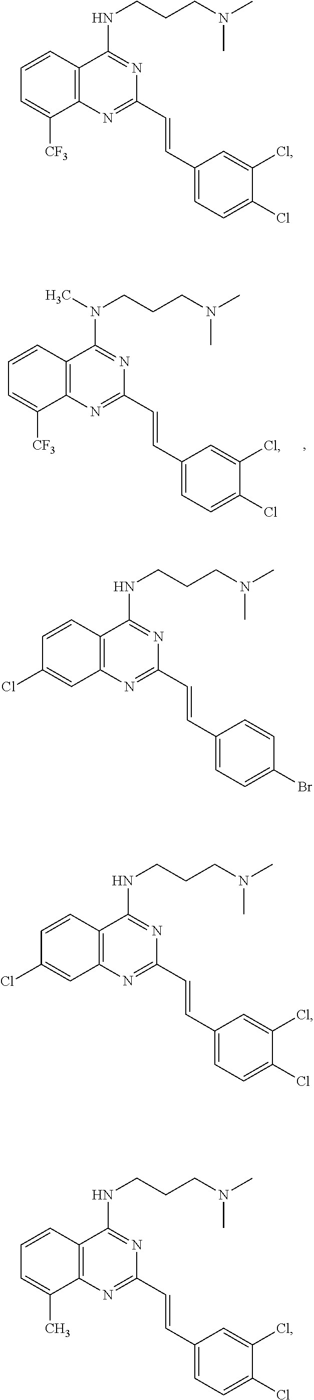 [SMILES: CN(C)CCCNc1nc(/C=C/c2ccc(Cl)c(Cl)c2)nc2c(C(F)(F)F)cccc12 ; CN(C)CCCN(C)c1nc(/C=C/c2ccc(Cl)c(Cl)c2)nc2c(C(F)(F)F)cccc12 ; CN(C)CCCNc1nc(/C=C/c2ccc(Br)cc2)nc2cc(Cl)ccc12 ; CN(C)CCCNc1nc(/C=C/c2ccc(Cl)c(Cl)c2)nc2cc(Cl)ccc12 ; Cc1cccc2c(NCCCN(C)C)nc(/C=C/c3ccc(Cl)c(Cl)c3)nc12]

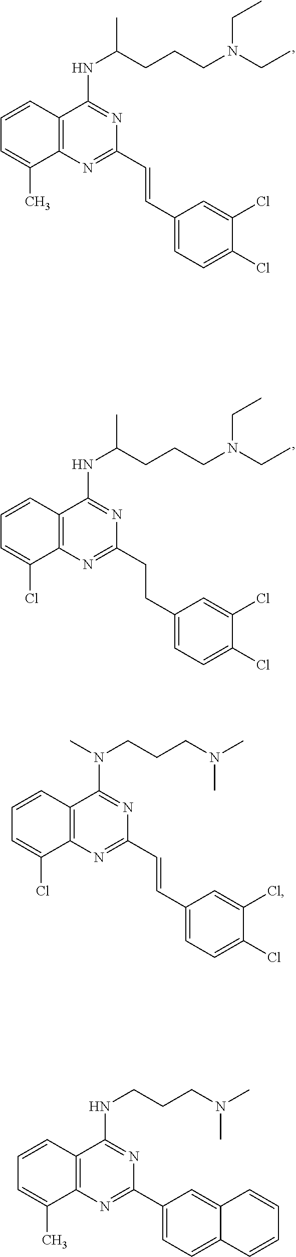 [SMILES: CCN(CC)CCCC(C)Nc1nc(/C=C/c2ccc(Cl)c(Cl)c2)nc2c(C)cccc12 ; CCN(CC)CCCC(C)Nc1nc(CCc2ccc(Cl)c(Cl)c2)nc2c(Cl)cccc12 ; CN(C)CCCN(C)c1nc(/C=C/c2ccc(Cl)c(Cl)c2)nc2c(Cl)cccc12 ; Cc1cccc2c(NCCCN(C)C)nc(-c3ccc4ccccc4c3)nc12]

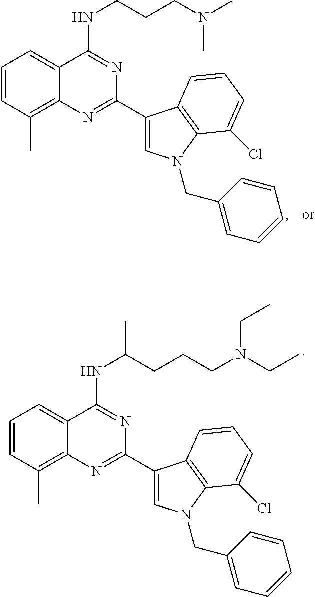 [SMILES: Cc1cccc2c(NCCCN(C)C)nc(-c3cn(Cc4ccccc4)c4c(Cl)cccc34)nc12 ; CCN(CC)CCCC(C)Nc1nc(-c2cn(Cc3ccccc3)c3c(Cl)cccc23)nc2c(C)cccc12]

In some embodiments, the invention is a method of treating a microbial infection in a subject in need thereof, the method comprising administering an antimicrobial agent and a compound to the subject, wherein the compound is selected from:

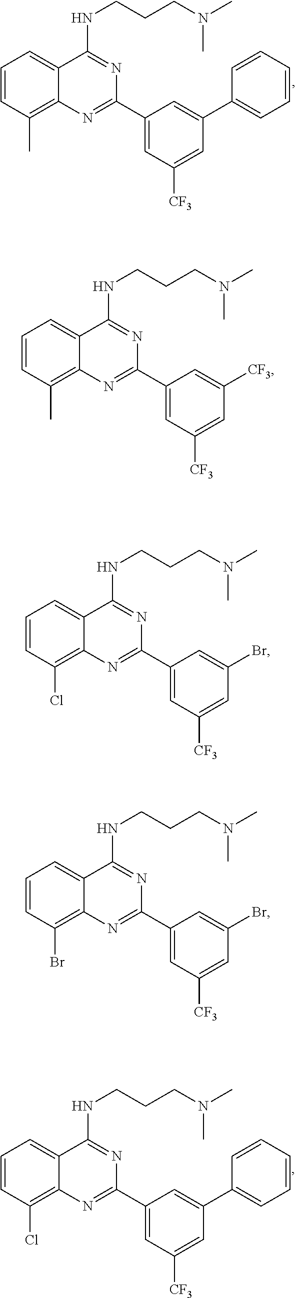 [SMILES: Cc1cccc2c(NCCCN(C)C)nc(-c3cc(-c4ccccc4)cc(C(F)(F)F)c3)nc12 ; Cc1cccc2c(NCCCN(C)C)nc(-c3cc(C(F)(F)F)cc(C(F)(F)F)c3)nc12 ; CN(C)CCCNc1nc(-c2cc(Br)cc(C(F)(F)F)c2)nc2c(Cl)cccc12 ; CN(C)CCCNc1nc(-c2cc(Br)cc(C(F)(F)F)c2)nc2c(Br)cccc12 ; CN(C)CCCNc1nc(-c2cc(-c3ccccc3)cc(C(F)(F)F)c2)nc2c(Cl)cccc12]

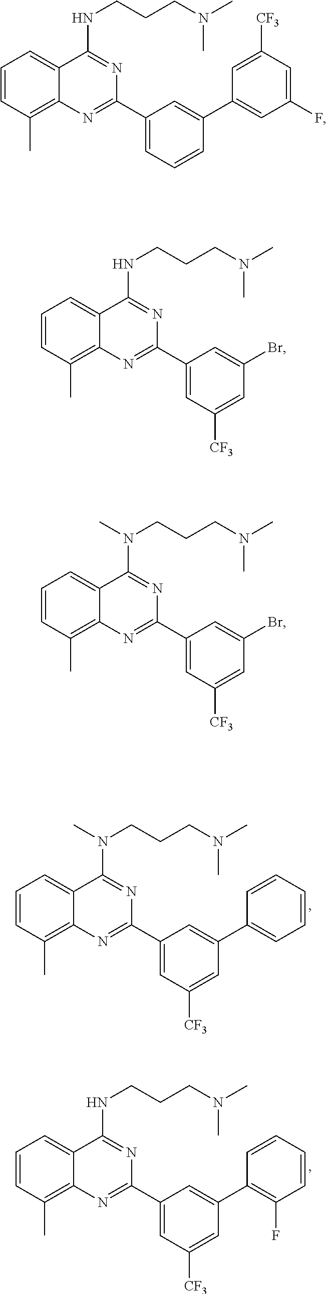 [SMILES: Cc1cccc2c(NCCCN(C)C)nc(-c3cccc(-c4cc(F)cc(C(F)(F)F)c4)c3)nc12 ; Cc1cccc2c(NCCCN(C)C)nc(-c3cc(Br)cc(C(F)(F)F)c3)nc12 ; Cc1cccc2c(N(C)CCCN(C)C)nc(-c3cc(Br)cc(C(F)(F)F)c3)nc12 ; Cc1cccc2c(N(C)CCCN(C)C)nc(-c3cc(-c4ccccc4)cc(C(F)(F)F)c3)nc12 ; Cc1cccc2c(NCCCN(C)C)nc(-c3cc(-c4ccccc4F)cc(C(F)(F)F)c3)nc12]

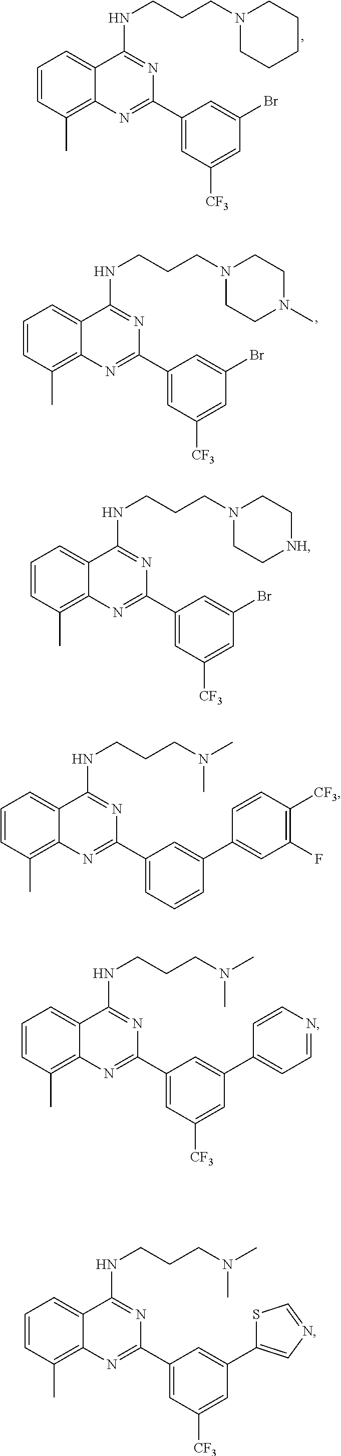

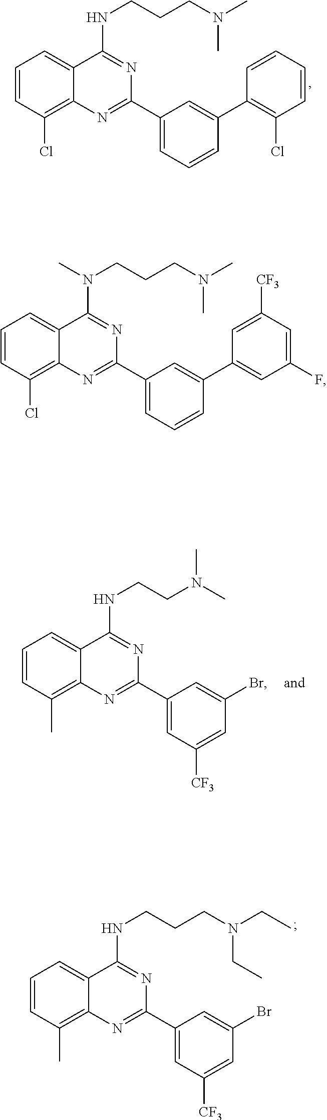 [SMILES: CN(C)CCCNc1nc(-c2cccc(-c3ccccc3Cl)c2)nc2c(Cl)cccc12 ; CN(C)CCCN(C)c1nc(-c2cccc(-c3cc(F)cc(C(F)(F)F)c3)c2)nc2c(Cl)cccc12 ; Cc1cccc2c(NCCN(C)C)nc(-c3cc(Br)cc(C(F)(F)F)c3)nc12 ; "and" ; CCN(CC)CCCNc1nc(-c2cc(Br)cc(C(F)(F)F)c2)nc2c(C)cccc12]

or a tautomer or a pharmaceutically acceptable salt thereof.

In other embodiments, the invention is a method of treating a microbial infection in a subject in need thereof, the method comprising administering a quinolone, fluoroquinolone, or a beta-lactam antibiotic and a compound of formula I, formula II, or formula III. Quinolone antibiotics include and are not limited to nalidixic acid. In some embodiments, the fluoroquinolone antibiotics include and are not limited to ciprofloxacin, levofloxacin, moxifloxacin, lomifloxacin, norfloxacin, ofloxacin, sparfloxacin, or trovafloxacin.

In particular embodiments, the invention is a method of treating a microbial infection in a subject in need thereof, the method comprising administering an antimicrobial agent and a compound of formula II or formula III, or tautomers or pharmaceutically acceptable salts thereof to the subject, the compound of formula II or formula III being:

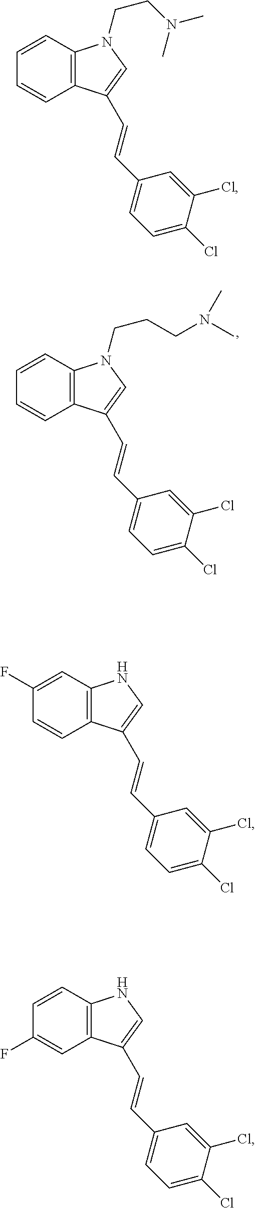

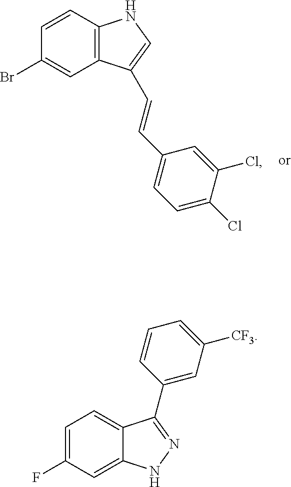 [SMILES: Clc1ccc(/C=C/c2c[nH]c3ccc(Br)cc23)cc1Cl ; "or" ; Fc1ccc2c(-c3cccc(C(F)(F)F)c3)n[nH]c2c1]

In some embodiments, the invention is a method of treating a microbialP1 infection in a subject in need thereof, the method comprising administering an antimicrobial agent and a compound to the subject, wherein the compound is selected from:

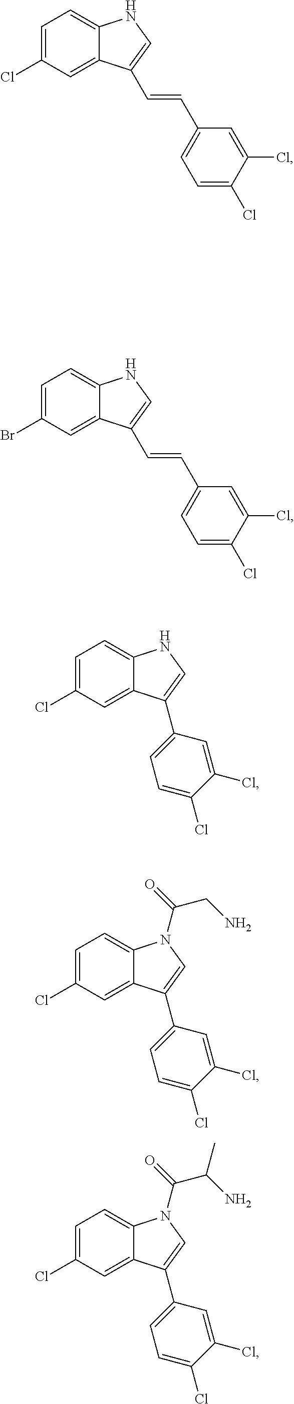

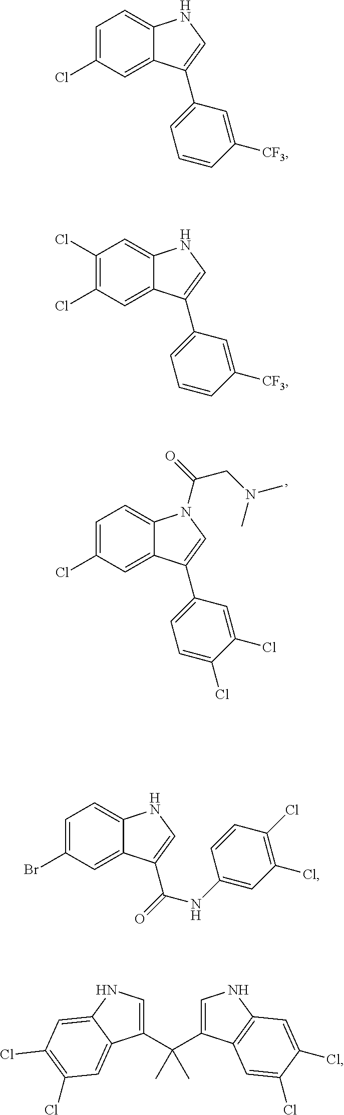 [SMILES: FC(F)(F)c1cccc(-c2c[nH]c3ccc(Cl)cc23)c1 ; FC(F)(F)c1cccc(-c2c[nH]c3cc(Cl)c(Cl)cc23)c1 ; CN(C)CC(=O)n1cc(-c2ccc(Cl)c(Cl)c2)c2cc(Cl)ccc21 ; O=C(Nc1ccc(Cl)c(Cl)c1)c1c[nH]c2ccc(Br)cc12 ; CC(C)(c1c[nH]c2cc(Cl)c(Cl)cc12)c1c[nH]c2cc(Cl)c(Cl)cc12]

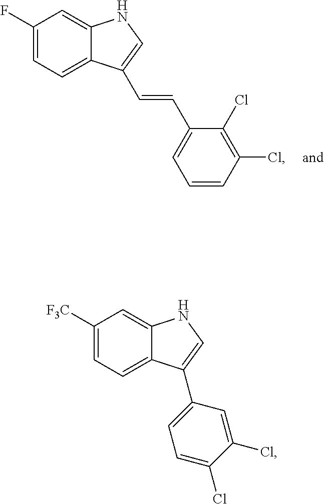 [SMILES: Fc1ccc2c(/C=C/c3cccc(Cl)c3Cl)c[nH]c2c1 ; "and" ; FC(F)(F)c1ccc2c(-c3ccc(Cl)c(Cl)c3)c[nH]c2c1]

or a tautomer or a stereoisomer or a pharmaceutically acceptable salt thereof.

In some embodiments, the invention is a method of increasing the activity of an antimicrobial agent, the method comprising administering the antimicrobial agent in combination with at least one compound that potentiates the activity of the antimicrobial agent. In one embodiment, the at least one compound that potentiates the activity of the antimicrobial agent inhibits RecA. In another embodiment, the at least one compound that potentiates the activity of the antimicrobial agent inhibits RecA-mediated strand exchange. In yet other embodiments, the at least one compound that potentiates the activity of the antimicrobial agent inhibits the SOS response in bacteria. In particular embodiments, the at least one compound that inhibits the SOS response in bacteria is:

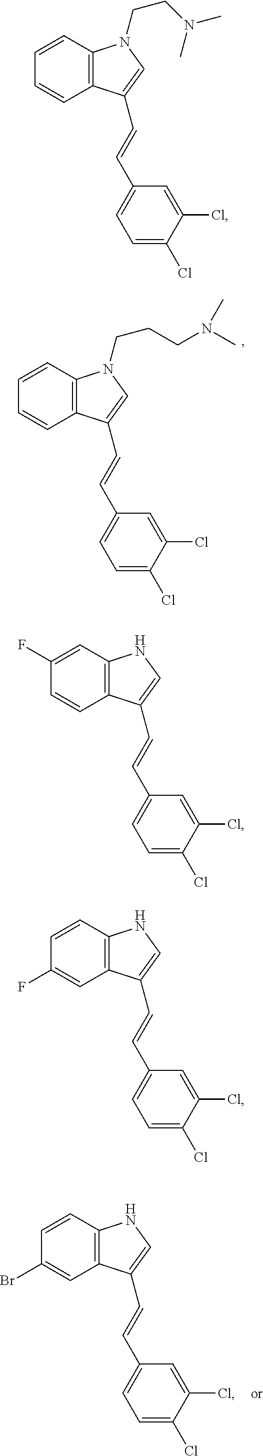 [SMILES: CN(C)CCn1cc(/C=C/c2ccc(Cl)c(Cl)c2)c2ccccc21 ; CN(C)CCCn1cc(/C=C/c2ccc(Cl)c(Cl)c2)c2ccccc21 ; Fc1ccc2c(/C=C/c3ccc(Cl)c(Cl)c3)c[nH]c2c1 ; Fc1ccc2[nH]cc(/C=C/c3ccc(Cl)c(Cl)c3)c2c1 ; Clc1ccc(/C=C/c2c[nH]c3ccc(Br)cc23)cc1Cl]

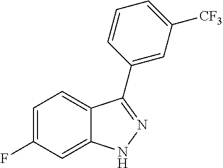 [SMILES: Fc1ccc2c(-c3cccc(C(F)(F)F)c3)n[nH]c2c1]

In some embodiments, the at least one compound that inhibits the SOS response in bacteria is selected from:

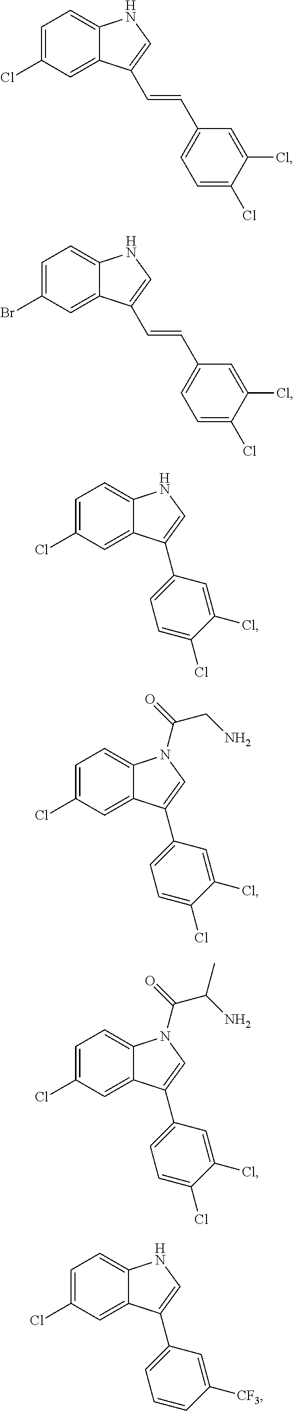

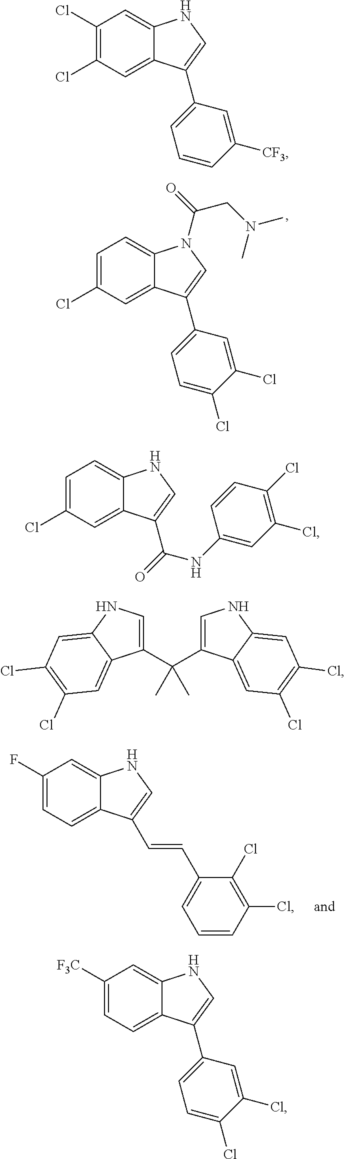

or a tautomer or a stereoisomer or a pharmaceutically acceptable salt thereof. In some embodiments, the at least one compound that potentiates the activity of the antimicrobial agent does not inhibit the SOS response in bacteria.

In a particular embodiment, the invention is a method of increasing the activity of an antimicrobial agent, the method comprising administering the antimicrobial agent in combination with at least one compound that potentiates the activity of the antimicrobial agent and potentiates the activity of colistin, and does not inhibit RecA. In particular embodiments, the compound that potentiates the activity of colistin is:

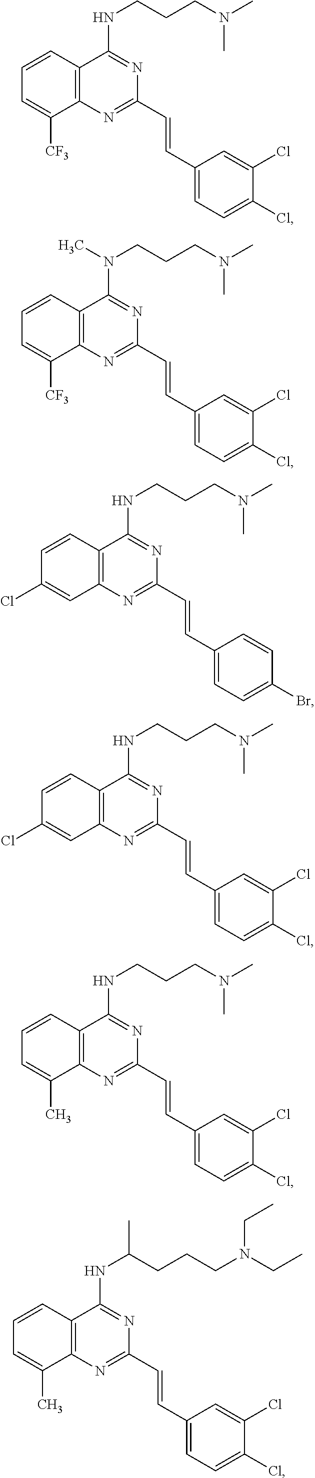

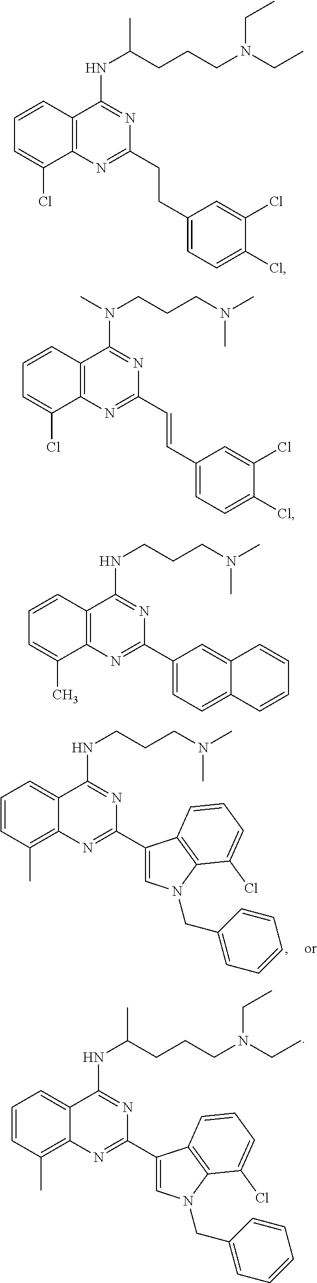

In some embodiments, the compound that potentiates the activity of colistin is selected from:

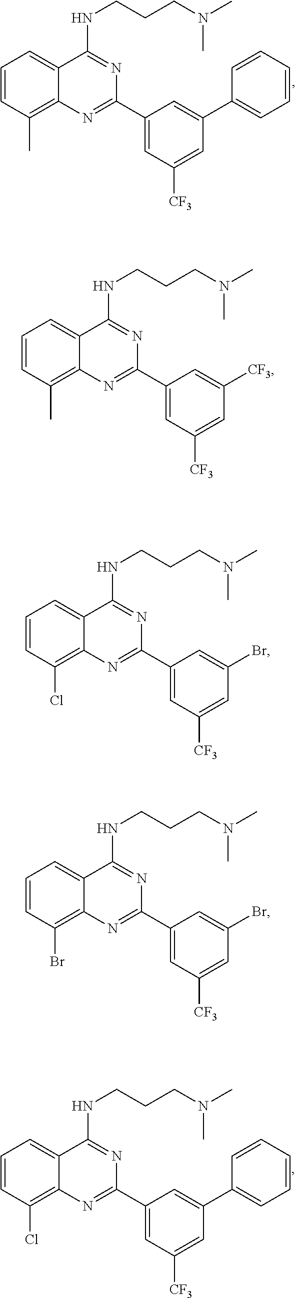 [SMILES: Cc1cccc2c(NCCCN(C)C)nc(-c3cc(-c4ccccc4)cc(C(F)(F)F)c3)nc12 ; Cc1cccc2c(NCCCN(C)C)nc(-c3cc(C(F)(F)F)cc(C(F)(F)F)c3)nc12 ; CN(C)CCCNc1nc(-c2cc(Br)cc(C(F)(F)F)c2)nc2c(Cl)cccc12 ; CN(C)CCCNc1nc(-c2cc(Br)cc(C(F)(F)F)c2)nc2c(Br)cccc12 ; CN(C)CCCNc1nc(-c2cc(-c3ccccc3)cc(C(F)(F)F)c2)nc2c(Cl)cccc12]

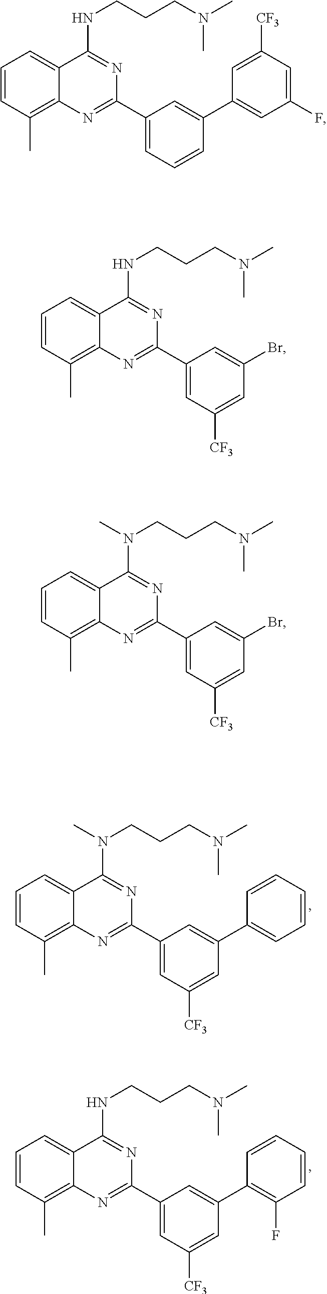 [SMILES: Cc1cccc2c(NCCCN(C)C)nc(-c3cccc(-c4cc(F)cc(C(F)(F)F)c4)c3)nc12 ; Cc1cccc2c(NCCCN(C)C)nc(-c3cc(Br)cc(C(F)(F)F)c3)nc12 ; Cc1cccc2c(N(C)CCCN(C)C)nc(-c3cc(Br)cc(C(F)(F)F)c3)nc12 ; Cc1cccc2c(N(C)CCCN(C)C)nc(-c3cc(-c4ccccc4)cc(C(F)(F)F)c3)nc12 ; Cc1cccc2c(NCCCN(C)C)nc(-c3cc(-c4ccccc4F)cc(C(F)(F)F)c3)nc12]

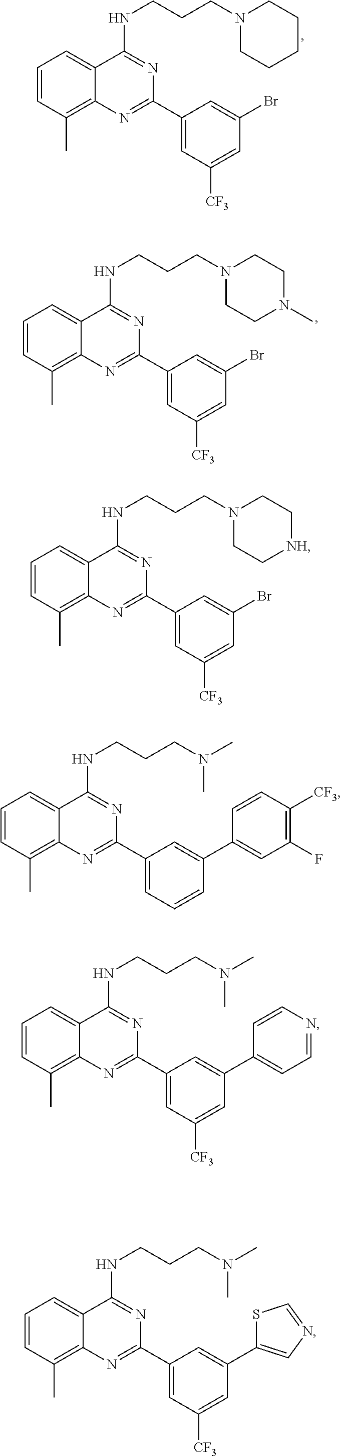

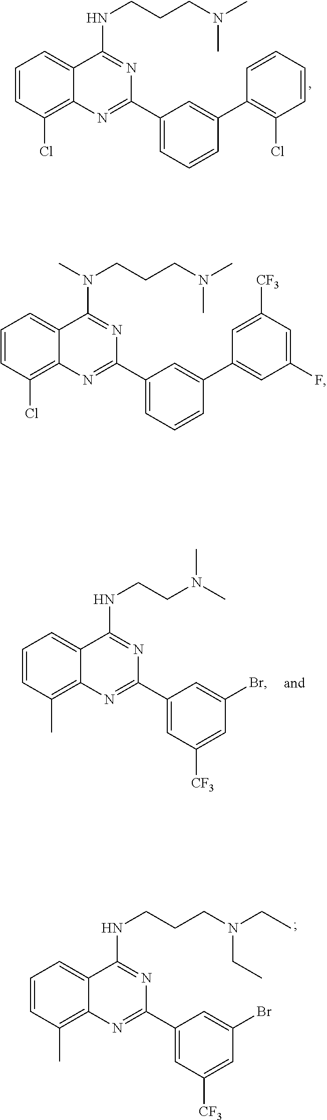 [SMILES: CN(C)CCCNc1nc(-c2cccc(-c3ccccc3Cl)c2)nc2c(Cl)cccc12 ; CN(C)CCCN(C)c1nc(-c2cccc(-c3cc(F)cc(C(F)(F)F)c3)c2)nc2c(Cl)cccc12 ; Cc1cccc2c(NCCN(C)C)nc(-c3cc(Br)cc(C(F)(F)F)c3)nc12 ; "and" ; CCN(CC)CCCNc1nc(-c2cc(Br)cc(C(F)(F)F)c2)nc2c(C)cccc12]

or a tautomer or a pharmaceutically acceptable salt thereof.

In some embodiments, the invention is a method of increasing the activity of an antimicrobial agent, the method comprising administering the antimicrobial agent in combination with at least one compound of formula Ia, formula IIa, or formula IIIa:

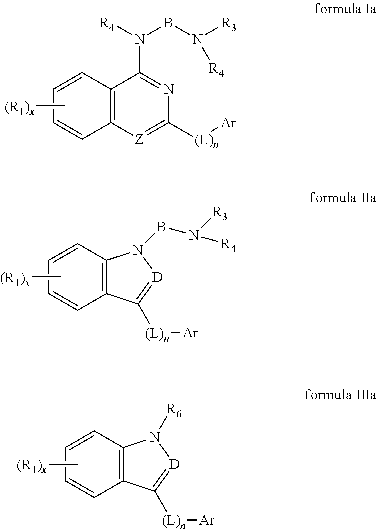

wherein,
each L is independently selected from the group consisting of C₁-C₆ alkylene, C₂-C₆ alkenylene, C₂-C₆ alkynylene, —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —NR₇C(O)—, —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-,
n is 0 or 1,
Ar is aryl, optionally substituted with one to five R₅ groups; or heteroaryl, optionally substituted with one to four R₅ groups,
B is an alkylene group, optionally interrupted by a carbonyl (C═O) or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,
D is CR₇ or N,
each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl, or optionally substituted heteroaryl; or two R₁ groups are linked together to form an optionally substituted 5- or 6-membered aromatic moiety or an optionally substituted 4- to 7-membered non-aromatic cyclic moiety,
R₂ is hydrogen, optionally substituted C₁-C₆ alkyl, C(O)R₇, or C₁-C₆-alkyloxycarbonyl,
R₃ and R₄ are independently hydrogen, optionally substituted C₁-C₆ alkyl, optionally substituted aryl, C(O)R₇, or C₁-C₆-alkyloxycarbonyl; or R₃ and R₄, together with the nitrogen to which they are attached, form a 4- to 6-membered heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇,
each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), optionally substituted heterocyclyl, or optionally substituted heteroaryl; and/or two R₅ groups are linked together to form an optionally substituted 5- or 6-membered aromatic moiety or an optionally substituted 4- to 7-membered non-aromatic cyclic moiety,
R₆ is hydrogen, optionally substituted C₁-C₆ alkyl, optionally substituted aryl-(C₁-C₆ alkylene), or C₁-C₆-alkyloxycarbonyl,
each R₇ is independently hydrogen, C₁-C₆ alkyl, or C₁-C₆ haloalkyl, and
each R₈ is independently C₁-C₆ alkyl or C₁-C₆ haloalkyl,
Z is CH or N,
x is 0, 1, 2, 3, or 4, and
if x is 0, Ar is not unsubstituted;
or a stereoisomer or a tautomer or a pharmaceutically acceptable salt thereof.

In some embodiments, the antimicrobial agent is a macrocyclic antibiotic, a quinolone antibiotic, a beta-lactam antibiotic, or an aminoglycoside antibiotic. In some embodiments, the antimicrobial agent is a macrocyclic antibiotic. In some embodiments, the macrocyclic antibiotic is a polymyxin antibiotic. In some embodiments, the polymyxin antibiotic is colistin. In some embodiments, the antimicrobial agent is a quinolone, fluoroquinolone, or beta-lactam antibiotic. In some embodiments, the antimicrobial agent is a quinolone antibiotic. In some embodiments, the antimicrobial agent is a fluoroquinolone antibiotic. In some embodiments, the antimicrobial agent is a beta-lactam antibiotic. In some embodiments, the fluoroquinolone antibiotic is ciprofloxacin, levofloxacin, moxifloxacin, lomifloxacin, norfloxacin, ofloxacin, sparfloxacin, or trovafloxacin.

In some embodiments, the invention is a method of increasing the activity of an antimicrobial agent, the method comprising administering the antimicrobial agent in combination with at least one compound of formula I, formula II, or formula III:

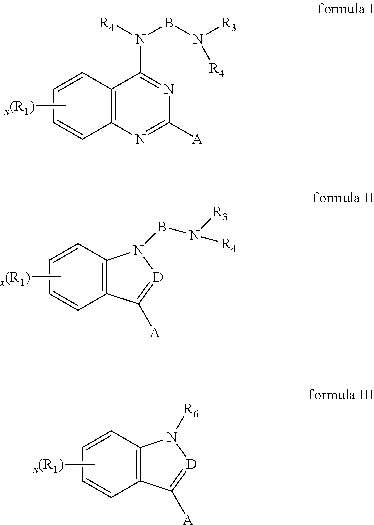

wherein,

A is one of the following groups:

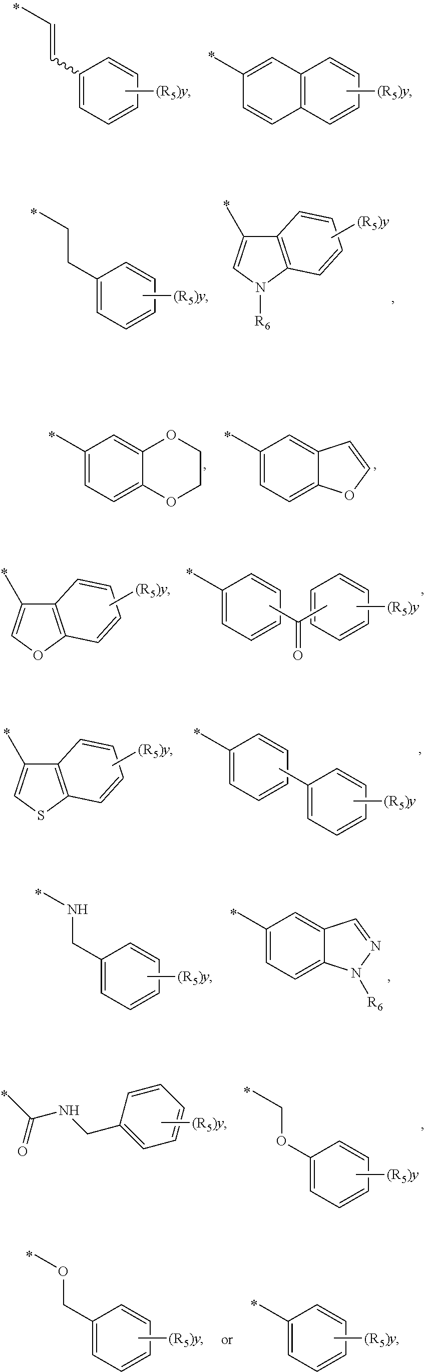

wherein * indicates the point of attachment of group A,

B is an alkylene group, optionally substituted by one to four groups, which are independently C₁-C₆ alkyl, C₁-C₆ haloalkyl, C₁-C₆ alkoxy, or C₁-C₆ haloalkoxy,

D is CH or a heteroatom,

each R₁ is independently halogen, C₁-C₆ alkyl, C₁-C₆ haloalkyl, nitro, or C₁-C₆-alkylamido,

each R₅ is independently halogen, C₁-C₆ alkyl, or C₁-C₆ haloalkyl,

R₂, R₃ and R₄ are independently hydrogen or optionally substituted C₁-C₆ alkyl, or C₁-C₆-alkyloxycarbonyl,

R₆ is hydrogen, optionally substituted C₁-C₆ alkyl, or C₁-C₆-alkyloxycarbonyl,

the wavy bond of formula A indicates that the double bond may be in a cis or trans configuration,

x is 0, 1, 2, 3, or 4,

y is 0, 1, 2, 3, 4, or 5, wherein both x and y are not simultaneously 0, and

and tautomers or pharmaceutically acceptable salts thereof.

In one embodiment, the invention is a method of increasing the activity of a macrocyclic antibiotic, comprises administering the macrocyclic antibiotic with a compound of formula I, formula II, or formula III. In some embodiments, macrocyclic antibiotic is polymyxin. In a particular embodiment, the polymyxin antibiotic is colistin.

In other embodiments, the invention is a method of increasing the activity of a quinolone, fluoroquinolone, or beta-lactam antibiotic, comprising administering the quinolone, fluoroquinolone, or beta-lactam antibiotic with a compound of formula I, formula II, or formula III. In one embodiment, the quinolone, fluoroquinolone, or beta-lactam antibiotic is a fluoroquinolone antibiotic. In a particular embodiment, the fluoroquinolone antibiotic is ciprofloxacin, levofloxacin, moxifloxacin, lomifloxacin, norfloxacin, ofloxacin, sparfloxacin, or trovafloxacin.

In some embodiments, the invention is a method of increasing the activity of an antimicrobial agent, the method comprising administering the antimicrobial agent in combination with at least one compound that potentiates the activity of the antimicrobial agent, wherein the at least one compound that potentiates the activity of the antimicrobial agent is a compound of formula I or formula II:

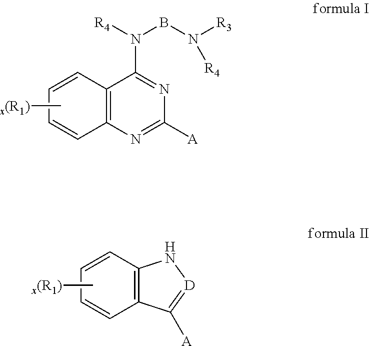

wherein,

A is one of the following groups:

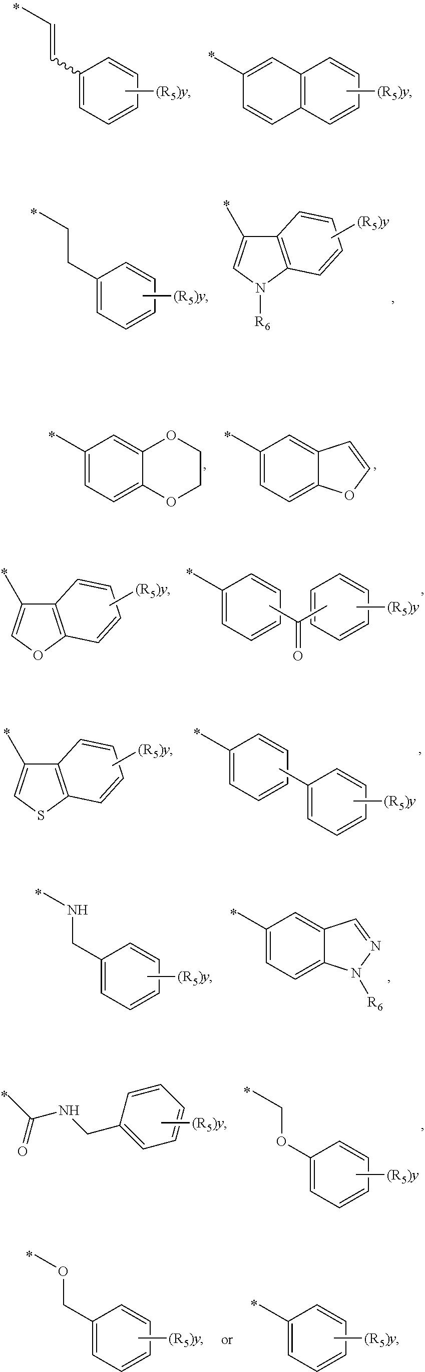

wherein * indicates the point of attachment of group A,

B is an alkylene group, optionally interrupted by a carbonyl (C═O) or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,

D is CH or a heteroatom,

each R₁ is independently halogen, C₁-C₆ alkyl, C₁-C₆ haloalkyl, nitro, or C₁-C₆-alkylamido,

each R₅ is independently halogen, C₁-C₆ alkyl, or C₁-C₆ haloalkyl,

R₂, R₃ and R₄ are independently hydrogen or optionally substituted C₁-C₆ alkyl, or C₁-C₆-alkyloxycarbonyl,

R₆ is hydrogen, optionally substituted C₁-C₆ alkyl, or C₁-C₆-alkyloxycarbonyl,

the wavy bond of formula A indicates that the double bond may be in a cis or trans configuration,

x is 0, 1, 2, 3, or 4,

y is 0, 1, 2, 3, 4, or 5, wherein both x and y are not simultaneously 0, and

or tautomers or pharmaceutically acceptable salts thereof.

In some embodiments, the antimicrobial agent is a macrocyclic antibiotic, a quinolone antibiotic, a beta-lactam antibiotic, or an aminoglycoside antibiotic. In some embodiments, the antimicrobial agent is a macrocyclic antibiotic. In some embodiments, the macrocyclic antibiotic is polymyxin. In some embodiments, the polymyxin antibiotic is colistin. In some embodiments, the antimicrobial agent is a quinolone, fluoroquinolone, or beta-lactam antibiotic. In some embodiments, the antimicrobial agent is a quinolone antibiotic. In some embodiments, the antimicrobial agent is a fluoroquinolone antibiotic. In some embodiments, the antimicrobial agent is a beta-lactam antibiotic.

In one aspect, the compounds of the invention decrease the minimum inhibitory concentration (MIC) of an antibiotic. Thus, in some embodiments, the invention is a method of decreasing the MIC of an antibiotic against a bacterium, comprising contacting the bacterium with the antibiotic and a compound of formula I, formula II, or formula III. In some embodiments, the invention is a method of decreasing the MIC of an antibiotic against a bacterium, comprising contacting the bacterium with the antibiotic and a compound of formula Ia, formula IIa, or formula IIIa. In some embodiments, the MIC of the antibiotic is decreased by about 5%, about 10%, about 15%, about 20%, about 25%, about 30%, about 35%, about 40%, about 45%, about 50%, about 55%, about 60%, about 65%, about 70%, about 75%, or about 80%. In some embodiments, the MIC of the antibiotic is decreased from about 10% to about 20%, from about 20% to about 30%, from about 30% to about 40%, from about 40% to about 50%, from about 50% to about 60%, from about 60% to about 70%, or from about 70% to about 80%. In some embodiments, the MIC of the antibiotic is decreased by about 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-70-, 75-, 80-, 85-, 90-, 95-, 100-, 125-, 150-, 175-, 200-, 225-, 250-, 275-, 300-, 325-, 350-, 375-400-, 425-, 450-, 475-, 500-, 525-, 550-, 575-, 600-, 625-, 650-, 675-, 700-, 725-, 750-, 775-, 800-, 825-, 850-, 875-, 900-, 925-, 950-, 975-, 1000-, 1100-, 1200-, 1300-, 1400-, 1500-, 1600-, 1700-, 1800-, 1900-, 2000-, 2500-, 3000-, 3500-, 4000-, 4500-, 5000-fold, or more, including increments therein. In some embodiments, the MIC of the antibiotic is decreased from about 2-fold to about 5000-fold, from about 2-fold to about 3500-fold, from about 2-fold to about 1000-fold, from about 10-fold to about 5000-fold, from about 10-fold to about 1000-fold. In some embodiments, the antimicrobial agent is a macrocyclic antibiotic, a quinolone antibiotic, a beta-lactam antibiotic, or an aminoglycoside antibiotic. In some embodiments, the antibiotic is a macrocyclic antibiotic. In some embodiments, the macrocyclic antibiotic is polymyxin. In a particular embodiment, the polymyxin antibiotic is colistin.

In one aspect, the compounds of the invention decrease the protective dose 50% (PD50) of an antibiotic. Thus, in some embodiments, the invention is a method of decreasing the PD50 of an antibiotic against a bacterium, comprising contacting the bacterium with the antibiotic and a compound of formula I, formula II, or formula III. In some embodiments, the invention is a method of decreasing the PD50 of an antibiotic against a bacterium, comprising contacting the bacterium with the antibiotic and a compound of formula Ia, formula IIa, or formula IIIa. In some embodiments, the PD50 of the antibiotic is decreased by about 5%, about 10%, about 15%, about 20%, about 25%, about 30%, about 35%, about 40%, about 45%, about 50%, about 55%, about 60%, about 65%, about 70%, about 75%, or about 80%. In some embodiments, the PD50 of the antibiotic is decreased from about 10% to about 20%, from about 20% to about 30%, from about 30% to about 40%, from about 40% to about 50%, from about 50% to about 60%, from about 60% to about 70%, or from about 70% to about 80%. In some embodiments, the PD50 of the antibiotic is decreased by about 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100-, 125-, 150-, 175-, 200-, 225-, 250-, 275-, 300-, 325-, 350-, 375-, 400-, 425-, 450-, 475-, 500-, 525-, 550-, 575-, 600-, 625-, 650-, 675-, 700-, 725-, 750-, 775-, 800-, 825-, 850-, 875-, 900-, 925-, 950-, 975-, 1000-, 1100-, 1200-, 1300-, 1400-, 1500-, 1600-, 1700-, 1800-, 1900-, 2000-, 2500-, 3000-, 3500-, 4000-, 4500-, 5000-fold, or more, including increments therein. In some embodiments, the PD50 of the antibiotic is decreased from about 2-fold to about 5000-fold, from about 2-fold to about 3500-fold, from about 2-fold to about 1000-fold, from about 10-fold to about 5000-fold, from about 10-fold to about 1000-fold. In some embodiments, the antimicrobial agent is a macrocyclic antibiotic, a quinolone antibiotic, a beta-lactam antibiotic, or an aminoglycoside antibiotic. In some embodiments, the antibiotic is a macrocyclic antibiotic. In some embodiments, the macrocyclic antibiotic is polymyxin. In a particular embodiment, the polymyxin antibiotic is colistin.

### Compositions

In some embodiments, the invention is a composition comprising at least one compound of formula Ia, formula IIa, or formula IIIa:

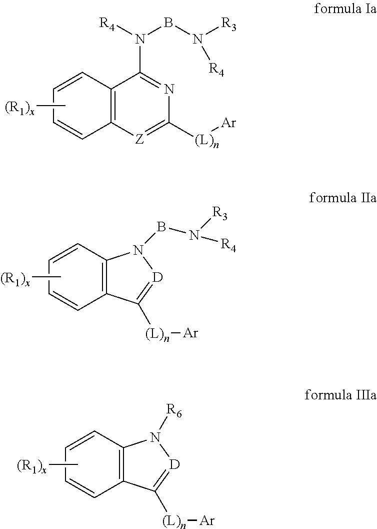

wherein,
each L is independently selected from the group consisting of C₁-C₆ alkylene, C₂-C₆ alkenylene, C₂-C₆ alkynylene, —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —NR₇C(O)—, —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-,
n is 0 or 1,
Ar is aryl, optionally substituted with one to five R₅ groups; or heteroaryl, optionally substituted with one to four R₅ groups,
B is an alkylene group, optionally interrupted by an oxygen, a carbonyl (C═O), or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,
D is CR₇ or N,
each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl, or optionally substituted heteroaryl; or two R₁ groups are linked together to form an optionally substituted 5- or 6-membered aromatic moiety or an optionally substituted 4- to 7-membered non-aromatic cyclic moiety,
R₂ is hydrogen, optionally substituted C₁-C₆ alkyl, C(O)R₇, or C₁-C₆-alkyloxycarbonyl,
R₃ and R₄ are independently hydrogen, optionally substituted C₁-C₆ alkyl, optionally substituted aryl, C(O)R₇, or C₁-C₆-alkyloxycarbonyl; or R₃ and R₄, together with the nitrogen to which they are attached, form a 4- to 6-membered heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇,
each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), optionally substituted heterocyclyl, optionally substituted C₃-C₇ cycloalkyl, or optionally substituted heteroaryl; and/or two R₅ groups are linked together to form an optionally substituted 5- or 6-membered aromatic moiety or an optionally substituted 4- to 7-membered non-aromatic cyclic moiety,
R₆ is hydrogen, optionally substituted C₁-C₆ alkyl, optionally substituted aryl-(C₁-C₆ alkylene), optionally substituted C₁-C₆-alkylcarbonyl, or C₁-C₆-alkyloxycarbonyl,
each R₇ is independently hydrogen, C₁-C₆ alkyl, or C₁-C₆ haloalkyl, and
each R₈ is independently C₁-C₆ alkyl or C₁-C₆ haloalkyl,
Z is CH or N,
x is 0, 1, 2, 3, or 4, and
if x is 0, Ar is not unsubstituted;

or a stereoisomer or a tautomer or a pharmaceutically acceptable salt thereof.

In some embodiments, the invention is a composition comprising at least one compound selected from Tables 1-15.

In some embodiments, the invention is a composition comprising an antimicrobial agent and at least one compound of formula Ia, formula IIa, or formula IIIa:

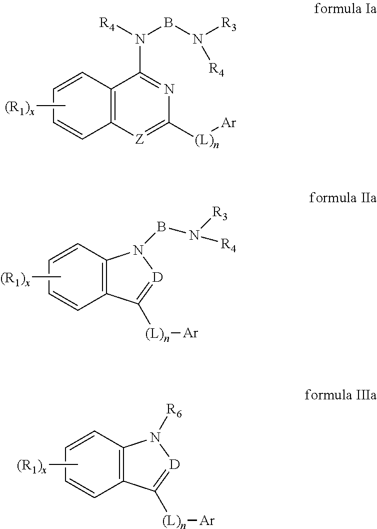

wherein,
each L is independently selected from the group consisting of C₁-C₆ alkylene, C₂-C₆ alkenylene, C₂-C₆ alkynylene, —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —NR₇C(O)—, —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-,
n is 0 or 1,
Ar is aryl, optionally substituted with one to five R₅ groups; or heteroaryl, optionally substituted with one to four R₅ groups,
B is an alkylene group, optionally interrupted by an oxygen, a carbonyl (C═O), or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,
D is CR₇ or N,
each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl, or optionally substituted heteroaryl; or two R₁ groups are linked together to form an optionally substituted 5- or 6-membered aromatic moiety or an optionally substituted 4- to 7-membered non-aromatic cyclic moiety,
R₂ is hydrogen, optionally substituted C₁-C₆ alkyl, C(O)R₇, or C₁-C₆-alkyloxycarbonyl,
R₃ and R₄ are independently hydrogen, optionally substituted C₁-C₆ alkyl, optionally substituted aryl, C(O)R₇, or C₁-C₆-alkyloxycarbonyl; or R₃ and R₄, together with the nitrogen to which they are attached, form a 4- to 6-membered heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇,
each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), optionally substituted heterocyclyl, optionally substituted C₃-C₇ cycloalkyl, or optionally substituted heteroaryl; and/or two R₅ groups are linked together to form an optionally substituted 5- or 6-membered aromatic moiety or an optionally substituted 4- to 7-membered non-aromatic cyclic moiety,
R₆ is hydrogen, optionally substituted C₁-C₆ alkyl, optionally substituted aryl-(C₁-C₆ alkylene), optionally substituted C₁-C₆-alkylcarbonyl, or C₁-C₆-alkyloxycarbonyl,
each R₇ is independently hydrogen, C₁-C₆ alkyl, or C₁-C₆ haloalkyl, and
each R₈ is independently C₁-C₆ alkyl or C₁-C₆ haloalkyl,
Z is CH or N,
x is 0, 1, 2, 3, or 4, and
if x is 0, Ar is not unsubstituted;

or a stereoisomer or a tautomer or a pharmaceutically acceptable salt thereof.

In some embodiments, the invention is a composition comprising an antimicrobial agent and at least one compound of formula Ia, formula IIa, or formula IIIa:

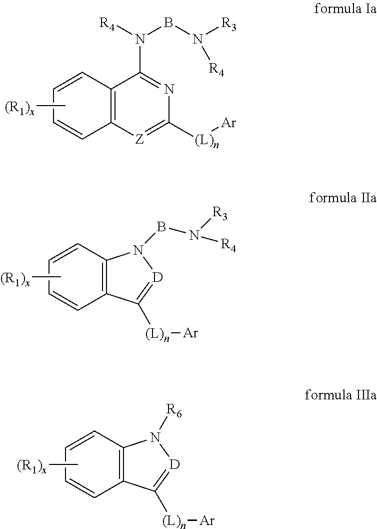

wherein,
each L is independently selected from the group consisting of C₁-C₆ alkylene, C₂-C₆ alkenylene, C₂-C₆ alkynylene, —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —NR₇C(O)—, —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-,
n is 0 or 1,
Ar is aryl, optionally substituted with one to five R₅ groups; or heteroaryl, optionally substituted with one to four R₅ groups,
B is an alkylene group, optionally interrupted by a carbonyl (C═O) or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,
D is CR₇ or N,
each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl, or optionally substituted heteroaryl; or two R₁ groups are linked together to form an optionally substituted 5- or 6-membered aromatic moiety or an optionally substituted 4- to 7-membered non-aromatic cyclic moiety,
R₂ is hydrogen, optionally substituted C₁-C₆ alkyl, C(O)R₇, or C₁-C₆-alkyloxycarbonyl,
R₃ and R₄ are independently hydrogen, optionally substituted C₁-C₆ alkyl, optionally substituted aryl, C(O)R₇, or C₁-C₆-alkyloxycarbonyl; or R₃ and R₄, together with the nitrogen to which they are attached, form a 4- to 6-membered heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇,
each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), optionally substituted heterocyclyl, or optionally substituted heteroaryl; and/or two R₅ groups are linked together to form an optionally substituted 5- or 6-membered aromatic moiety or an optionally substituted 4- to 7-membered non-aromatic cyclic moiety,
R₆ is hydrogen, optionally substituted C₁-C₆ alkyl, optionally substituted aryl-(C₁-C₆ alkylene), or C₁-C₆-alkyloxycarbonyl,
each R₇ is independently hydrogen, C₁-C₆ alkyl, or C₁-C₆ haloalkyl, and
each R₈ is independently C₁-C₆ alkyl or C₁-C₆ haloalkyl,
Z is CH or N,
x is 0, 1, 2, 3, or 4, and
if x is 0, Ar is not unsubstituted;

or a stereoisomer or a tautomer or a pharmaceutically acceptable salt thereof.

In some embodiments, the invention is a composition comprising at least one compound of formula Ia, formula IIa, or formula IIIa and a macrocyclic antibiotic. In another embodiment, the invention is a composition comprising at least one compound of formula Ia, formula IIa, or formula IIIa in a single dosage form and polymyxin. In a particular embodiment, the invention is a composition, comprising at least one compound of formula Ia, formula IIa, or formula IIIa and colistin.

In other embodiments, the invention is a composition, comprising at least one compound of formula Ia, formula IIa, or formula IIIa and an antimicrobial agent, which is a quinolone, fluoroquinolone, or beta-lactam antibiotic. In some embodiments, the antimicrobial agent is a fluoroquinolone antibiotic.

In other embodiments, the invention is a composition, comprising at least one compound of formula Ia, formula IIa, or formula IIIa and an antimicrobial agent, which is a macrocyclic antibiotic, a quinolone antibiotic, a beta-lactam antibiotic, or an aminoglycoside antibiotic. In some embodiments, the antimicrobial agent is a fluoroquinolone antibiotic.

In some embodiments, the invention is a composition comprising an antimicrobial agent and at least one compound of formula I, formula II, or formula III:

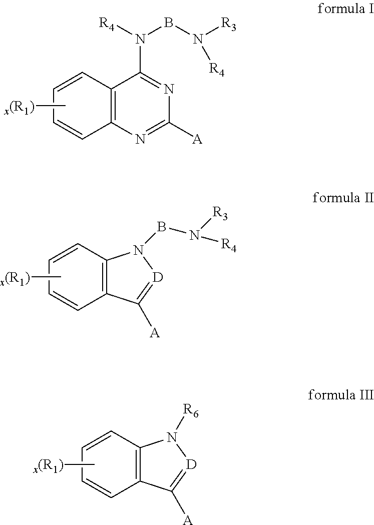

wherein

A is one of the following groups:

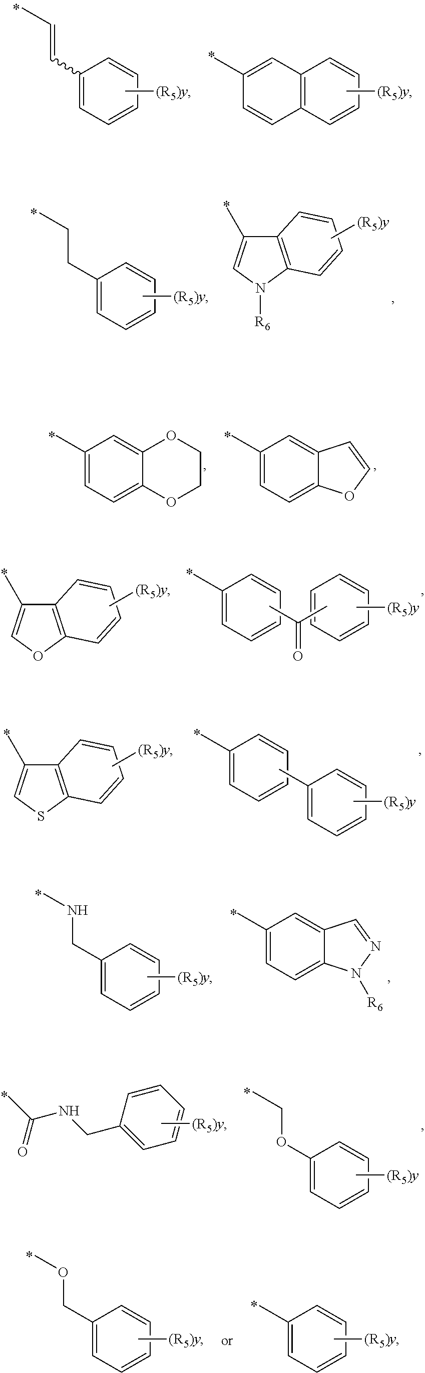

wherein * indicates the point of attachment of group A,

B is an alkylene group, optionally substituted by one to four groups, which are independently C₁-C₆ alkyl, C₁-C₆ haloalkyl, C₁-C₆ alkoxy, or C₁-C₆ haloalkoxy,

D is CH or a heteroatom,

each R₁ is independently halogen, C₁-C₆ alkyl, C₁-C₆ haloalkyl, nitro, or C₁-C₆-alkylamido,

each R₅ is independently halogen, C₁-C₆ alkyl, or C₁-C₆ haloalkyl,

R₂, R₃ and R₄ are independently hydrogen or optionally substituted C₁-C₆ alkyl, or C₁-C₆-alkyloxycarbonyl,

R₆ is hydrogen, optionally substituted C₁-C₆ alkyl, or C₁-C₆-alkyloxycarbonyl,

the wavy bond of formula A indicates that the double bond may be in a cis or trans configuration,

x is 0, 1, 2, 3, or 4,

y is 0, 1, 2, 3, 4, or 5, wherein both x and y are not simultaneously 0, and

and tautomers or pharmaceutically acceptable salts thereof.

In one embodiment, the invention is a composition comprising at least one compound of formula I, formula II, or formula III and a macrocyclic antibiotic. In another embodiment, the invention is a composition comprising at least one compound of formula I, formula II, or formula III in a single dosage form and polymyxin. In a particular embodiment, the invention is a composition, comprising at least one compound of formula I, formula II, or formula III and colistin.

In other embodiments, the invention is a composition, comprising at least one compound of formula I, formula II, or formula III and an antimicrobial agent, which is a quinolone, fluoroquinolone, or beta-lactam antibiotic. In some embodiments, the antimicrobial agent is a fluoroquinolone antibiotic.

In other embodiments, the invention is a composition, comprising at least one compound of formula I, formula II, or formula III and an antimicrobial agent, which is a macrocyclic antibiotic, a quinolone antibiotic, a beta-lactam antibiotic, or an aminoglycoside antibiotic. In some embodiments, the antimicrobial agent is a fluoroquinolone antibiotic.

In some embodiments, the invention is a composition comprising an antimicrobial agent and at least one compound of formula I or formula II:

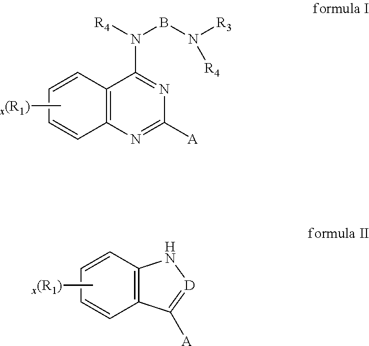

wherein

A is one of the following groups:

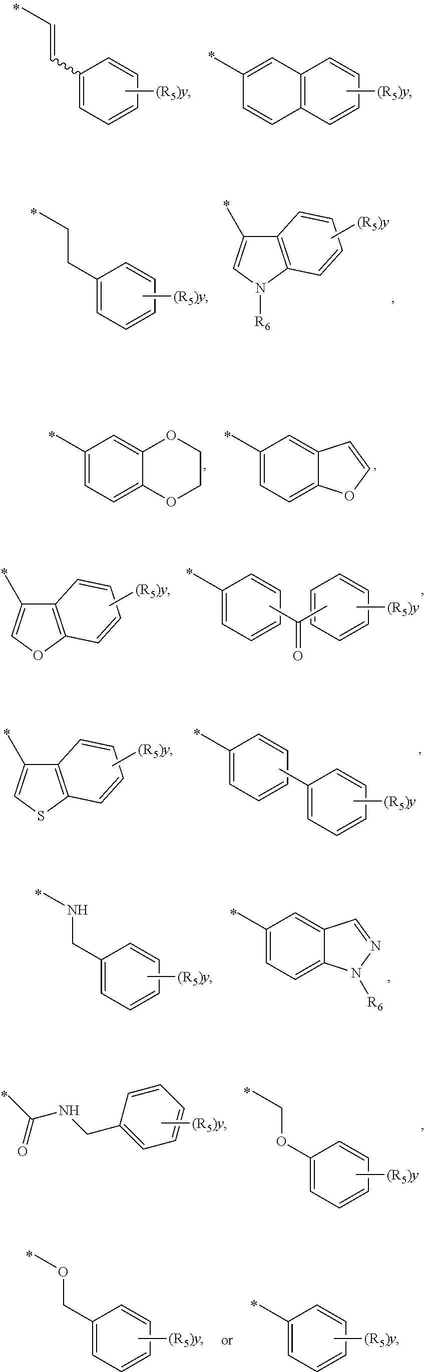

wherein * indicates the point of attachment of group A,

B is an alkylene group, optionally interrupted by a carbonyl (C═O) or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,

D is CH or a heteroatom,

each R₁ is independently halogen, C₁-C₆ alkyl, C₁-C₆ haloalkyl, nitro, or C₁-C₆-alkylamido,

each R₅ is independently halogen, C₁-C₆ alkyl, or C₁-C₆ haloalkyl,

R₂, R₃ and R₄ are independently hydrogen or optionally substituted C₁-C₆ alkyl, or C₁-C₆-alkyloxycarbonyl,

R₆ is hydrogen, optionally substituted C₁-C₆ alkyl, or C₁-C₆-alkyloxycarbonyl,

the wavy bond of formula A indicates that the double bond may be in a cis or trans configuration,

x is 0, 1, 2, 3, or 4,

y is 0, 1, 2, 3, 4, or 5, wherein both x and y are not simultaneously 0, and

or tautomers or pharmaceutically acceptable salts thereof.

In some embodiments, the antimicrobial agent is a macrocyclic antibiotic, a quinolone antibiotic, a beta-lactam antibiotic, or an aminoglycoside antibiotic. In some embodiments, the antimicrobial agent is a macrocyclic antibiotic. In some embodiments, the macrocyclic antibiotic is polymyxin. In some embodiments, the polymyxin antibiotic is colistin. In some embodiments, the antimicrobial agent is a quinolone, fluoroquinolone, or beta-lactam antibiotic. In some embodiments, the antimicrobial agent is a quinolone antibiotic. In some embodiments, the antimicrobial agent is a fluoroquinolone antibiotic. In some embodiments, the antimicrobial agent is a beta-lactam antibiotic.

In particular embodiments, the invention is a composition comprising an antimicrobial agent and at least one compound having the following structure:

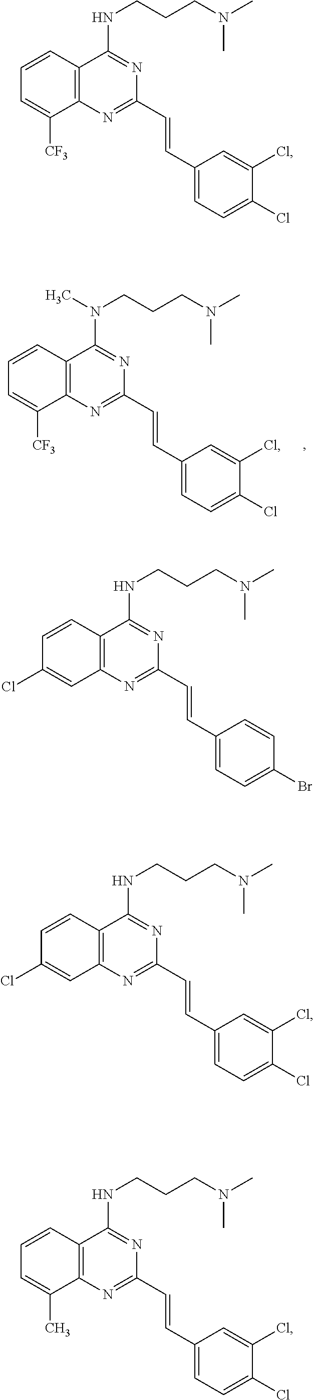 [SMILES: CN(C)CCCNc1nc(/C=C/c2ccc(Cl)c(Cl)c2)nc2c(C(F)(F)F)cccc12 ; CN(C)CCCN(C)c1nc(/C=C/c2ccc(Cl)c(Cl)c2)nc2c(C(F)(F)F)cccc12 ; CN(C)CCCNc1nc(/C=C/c2ccc(Br)cc2)nc2cc(Cl)ccc12 ; CN(C)CCCNc1nc(/C=C/c2ccc(Cl)c(Cl)c2)nc2cc(Cl)ccc12 ; Cc1cccc2c(NCCCN(C)C)nc(/C=C/c3ccc(Cl)c(Cl)c3)nc12]

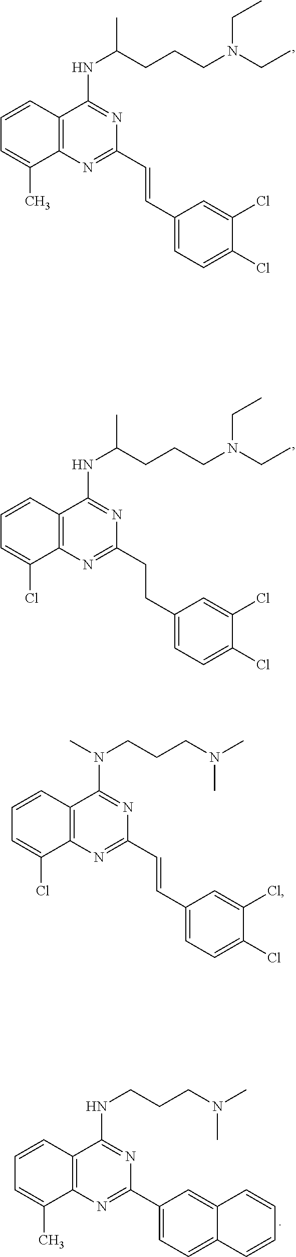 [SMILES: CCN(CC)CCCC(C)Nc1nc(/C=C/c2ccc(Cl)c(Cl)c2)nc2c(C)cccc12 ; CCN(CC)CCCC(C)Nc1nc(CCc2ccc(Cl)c(Cl)c2)nc2c(Cl)cccc12 ; CN(C)CCCN(C)c1nc(/C=C/c2ccc(Cl)c(Cl)c2)nc2c(Cl)cccc12 ; Cc1cccc2c(NCCCN(C)C)nc(-c3ccc4ccccc4c3)nc12]

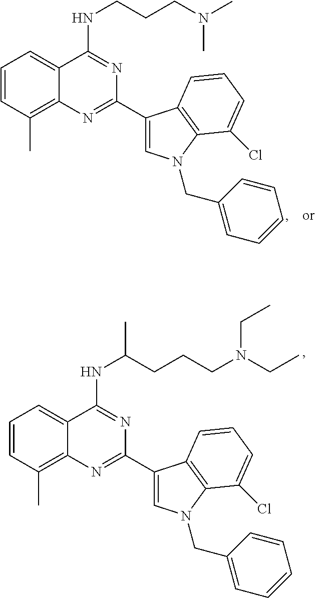 [SMILES: Cc1cccc2c(NCCCN(C)C)nc(-c3cn(Cc4ccccc4)c4c(Cl)cccc34)nc12 ; CCN(CC)CCCC(C)Nc1nc(-c2cn(Cc3ccccc3)c3c(Cl)cccc23)nc2c(C)cccc12]

or tautomers or pharmaceutically acceptable salts thereof.

In some embodiments, the invention is a composition comprising an antimicrobial agent and a compound selected from:

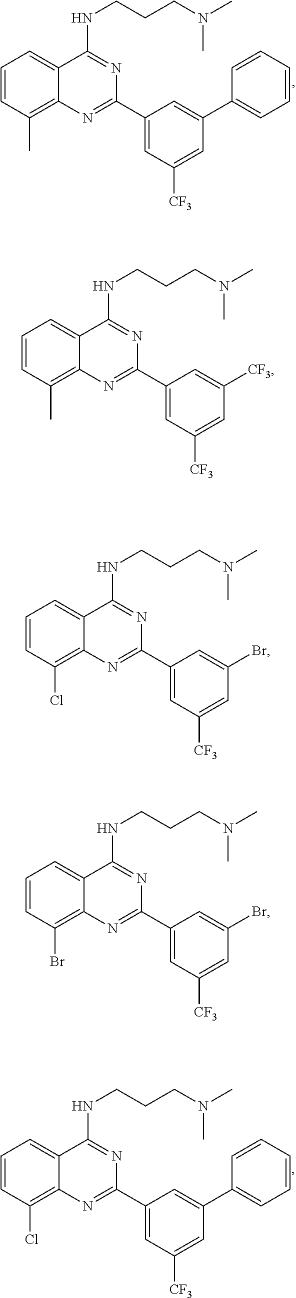 [SMILES: Cc1cccc2c(NCCCN(C)C)nc(-c3cc(-c4ccccc4)cc(C(F)(F)F)c3)nc12 ; Cc1cccc2c(NCCCN(C)C)nc(-c3cc(C(F)(F)F)cc(C(F)(F)F)c3)nc12 ; CN(C)CCCNc1nc(-c2cc(Br)cc(C(F)(F)F)c2)nc2c(Cl)cccc12 ; CN(C)CCCNc1nc(-c2cc(Br)cc(C(F)(F)F)c2)nc2c(Br)cccc12 ; CN(C)CCCNc1nc(-c2cc(-c3ccccc3)cc(C(F)(F)F)c2)nc2c(Cl)cccc12]

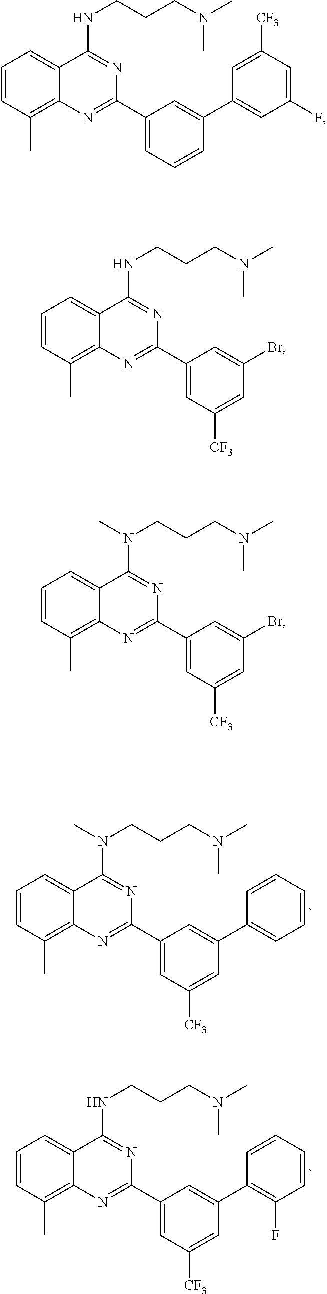 [SMILES: Cc1cccc2c(NCCCN(C)C)nc(-c3cccc(-c4cc(F)cc(C(F)(F)F)c4)c3)nc12 ; Cc1cccc2c(NCCCN(C)C)nc(-c3cc(Br)cc(C(F)(F)F)c3)nc12 ; Cc1cccc2c(N(C)CCCN(C)C)nc(-c3cc(Br)cc(C(F)(F)F)c3)nc12 ; Cc1cccc2c(N(C)CCCN(C)C)nc(-c3cc(-c4ccccc4)cc(C(F)(F)F)c3)nc12 ; Cc1cccc2c(NCCCN(C)C)nc(-c3cc(-c4ccccc4F)cc(C(F)(F)F)c3)nc12]

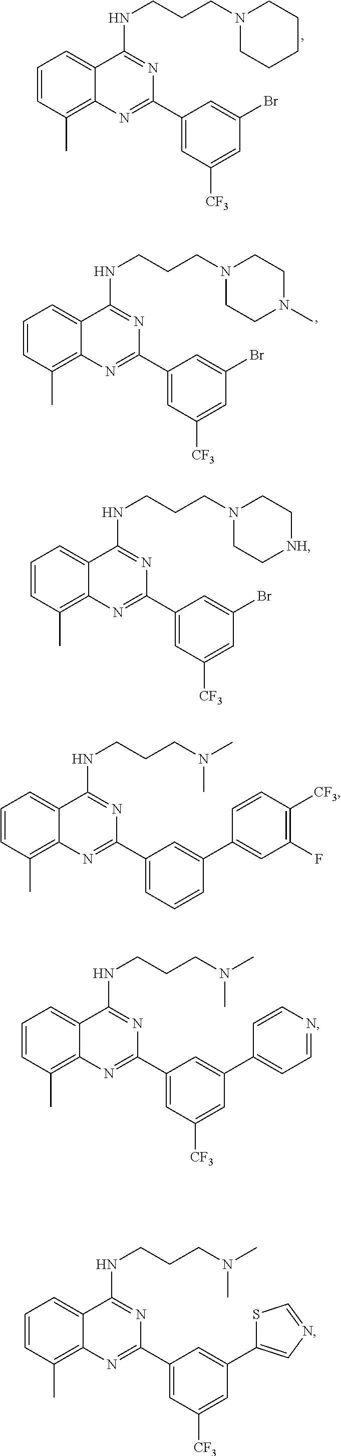

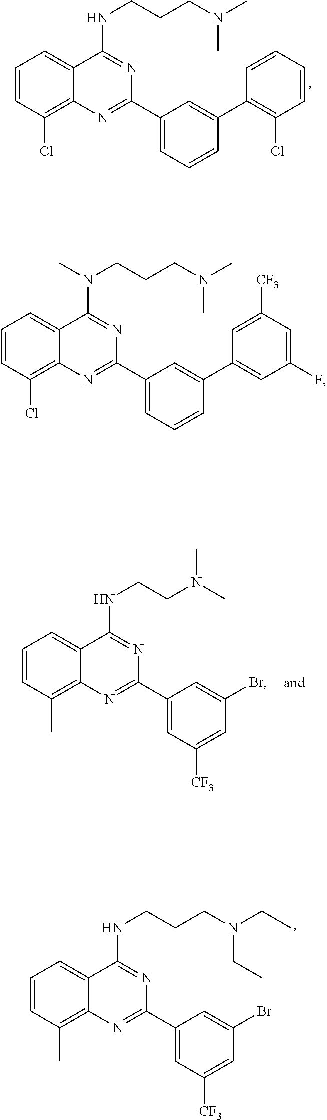 [SMILES: CN(C)CCCNc1nc(-c2cccc(-c3ccccc3Cl)c2)nc2c(Cl)cccc12 ; CN(C)CCCN(C)c1nc(-c2cccc(-c3cc(F)cc(C(F)(F)F)c3)c2)nc2c(Cl)cccc12 ; Cc1cccc2c(NCCN(C)C)nc(-c3cc(Br)cc(C(F)(F)F)c3)nc12 ; "and" ; CCN(CC)CCCNc1nc(-c2cc(Br)cc(C(F)(F)F)c2)nc2c(C)cccc12]

or a stereoisomer or a tautomer or a pharmaceutically acceptable salt thereof.

In other embodiments, the invention is a composition comprising an antimicrobial agent and a compound having the following structure:

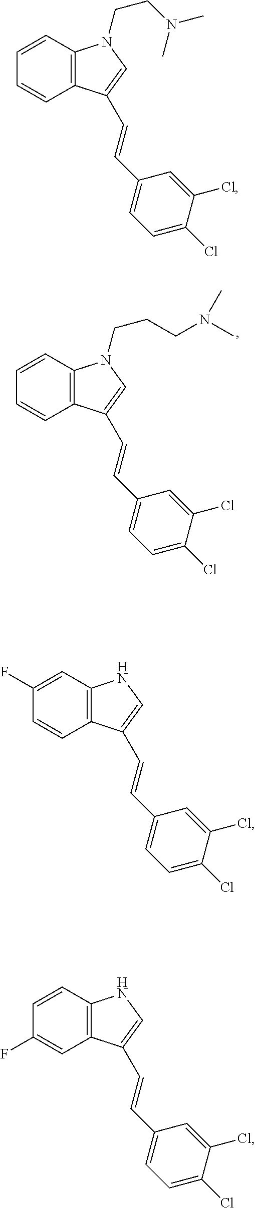

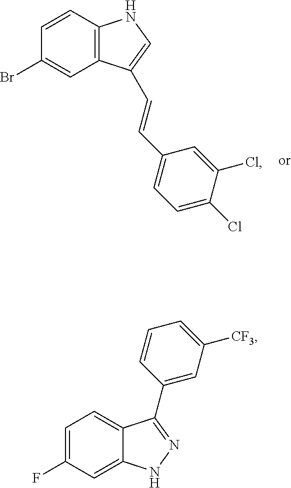 [SMILES: Clc1ccc(/C=C/c2c[nH]c3ccc(Br)cc23)cc1Cl ; "or" ; Fc1ccc2c(-c3cccc(C(F)(F)F)c3)n[nH]c2c1]

or tautomers or pharmaceutically acceptable salts thereof.

In some embodiments, the invention is a composition comprising an antimicrobial agent and a compound selected from:

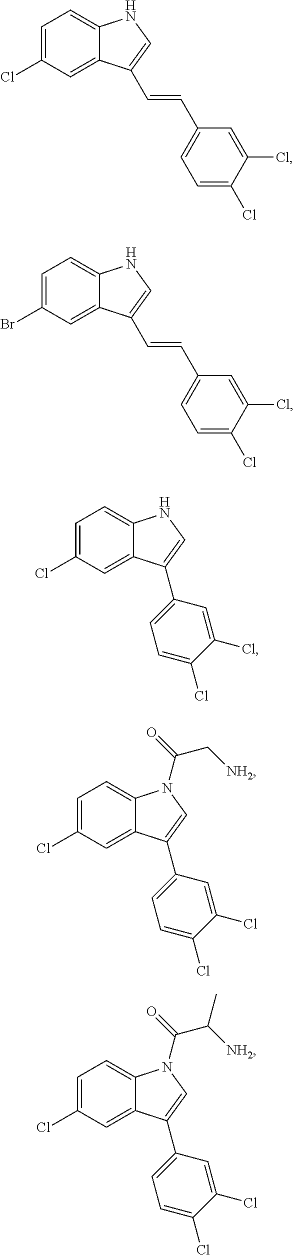

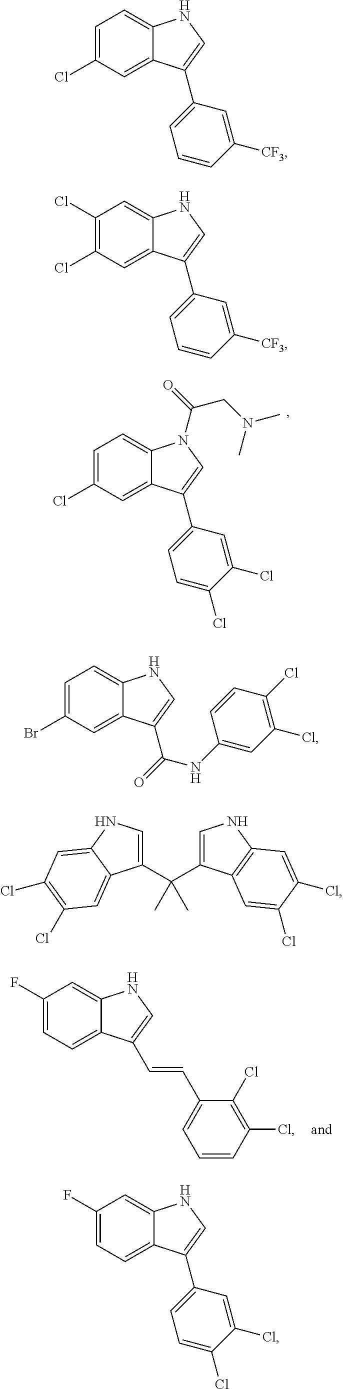

or a tautomer or a stereoisomer or a pharmaceutically acceptable salt thereof.

In some embodiments, the invention is a composition comprising an antimicrobial agent and at least one compound selected from Tables 1-15.

### Pharmaceutical Dosage Forms

When administered to a patient, a compound of the invention can be administered as a component of a composition that comprises a pharmaceutically acceptable carrier or excipient. A compound of the invention can be administered by any appropriate route, as determined by the medical practitioner. Methods of administration may include intradermal, intramuscular, intraperitoneal, parenteral, intravenous, subcutaneous, intranasal, epidural, oral, sublingual, buccal, intracerebral, intravaginal, transdermal, transmucosal, rectal, by inhalation, or topical (particularly to the ears, nose, eyes, or skin). Delivery can be either local or systemic. In certain embodiments, administration will result in the release of a compound of the invention into the bloodstream.

Pharmaceutical compositions of the invention can take the form of solutions, suspensions, emulsions, tablets, pills, pellets, powders, multi-particulates, capsules, capsules containing liquids, capsules containing powders, capsules containing multiparticulates, lozenges, sustained-release formulations, suppositories, transdermal patches, transmucosal films, sublingual tablets or tabs, aerosols, sprays, or any other form suitable for use. In one embodiment, the composition is in the form of a tablet. In another embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro ed., 19th ed. 1995), incorporated herein by reference.

Pharmaceutical compositions of the invention preferably comprise a suitable amount of a pharmaceutically acceptable excipient so as to provide the form for proper administration to the patient. Such a pharmaceutical excipient can be a diluent, suspending agent, solubilizer, binder, disintegrant, preservative, coloring agent, lubricant, and the like. The pharmaceutical excipient can be a liquid, such as water or an oil, including those of petroleum, animal, vegetable, or synthetic origin, such as peanut oil, soybean oil, mineral oil, sesame oil, and the like. The pharmaceutical excipient can be saline, gum acacia, gelatin, starch paste, talc, keratin, colloidal silica, urea, and the like. In addition, auxiliary, stabilizing, thickening, lubricating, and coloring agents can be used. In one embodiment, the pharmaceutically acceptable excipient is sterile when administered to a patient. Water is a particularly useful excipient when a compound of the invention is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The invention compositions, if desired, can also contain minor amounts of wetting or emulsifying agents, or pH buffering agents. Specific examples of pharmaceutically acceptable carriers and excipients that can be used to formulate oral dosage forms are described in the Handbook of Pharmaceutical Excipients, American Pharmaceutical Association (1986).

In certain embodiments, the compounds of the invention are formulated for oral administration. A compound of the invention to be orally delivered can be in the form of tablets, capsules, gelcaps, caplets, lozenges, aqueous or oily solutions, suspensions, granules, powders, emulsions, syrups, or elixirs, for example. When a compound of the invention is incorporated into oral tablets, such tablets can be compressed, tablet triturates, enteric-coated, sugar-coated, film-coated, multiply compressed or multiply layered.

An orally administered compound of the invention can contain one or more additional agents such as, for example, sweetening agents such as fructose, aspartame or saccharin; flavoring agents such as peppermint, oil of wintergreen, or cherry; coloring agents; and preserving agents, and stabilizers, to provide stable, pharmaceutically palatable dosage forms. Techniques and compositions for making solid oral dosage forms are described in Pharmaceutical Dosage Forms: Tablets (Lieberman, Lachman and Schwartz, eds., 2nd ed.) published by Marcel Dekker, Inc. Techniques and compositions for making tablets (compressed and molded), capsules (hard and soft gelatin) and pills are also described in Remington's Pharmaceutical Sciences 1553-1593 (Arthur Osol, ed., 16th ed., Mack Publishing, Easton, Pa. 1980). Liquid oral dosage forms include aqueous and nonaqueous solutions, emulsions, suspensions, and solutions and/or suspensions reconstituted from non-effervescent granules, optionally containing one or more suitable solvents, preservatives, emulsifying agents, suspending agents, diluents, sweeteners, coloring agents, flavoring agents, and the like. Techniques and compositions for making liquid oral dosage forms are described in Pharmaceutical Dosage Forms: Disperse Systems, (Lieberman, Rieger and Banker, eds.) published by Marcel Dekker, Inc.

When a compound of the invention is formulated for parenteral administration by injection (e.g., continuous infusion or bolus injection), the formulation can be in the form of a suspension, solution, or emulsion in an oily or aqueous vehicle, and such formulations can further comprise pharmaceutically necessary additives such as one or more stabilizing agents, suspending agents, dispersing agents, and the like. When a compound of the invention is to be injected parenterally, it can be, e.g., in the form of an isotonic sterile solution. A compound of the invention can also be in the form of a powder for reconstitution as an injectable formulation.

In certain embodiments, a compound of the invention can be delivered in an immediate release form. In other embodiments, a compound of the invention can be delivered in a controlled-release system or sustained-release system. Controlled- or sustained-release pharmaceutical compositions can have a common goal of improving drug therapy over the results achieved by their non-controlled or non-sustained-release counterparts. In one embodiment, a controlled- or sustained-release composition comprises a minimal amount of a compound of the invention to treat or prevent the condition (or a symptom thereof) in a minimum amount of time. Advantages of controlled- or sustained-release compositions include extended activity of the drug, reduced dosage frequency, and increased compliance. In addition, controlled- or sustained-release compositions can favorably affect the time of onset of action or other characteristics, such as blood levels of the compound of the invention, and can thus reduce the occurrence of adverse side effects.

Controlled- or sustained-release compositions can initially immediately release an amount of a compound of the invention that promptly produces the desired therapeutic or prophylactic effect, and gradually and continually release other amounts of the compound of the invention to maintain a level of therapeutic or prophylactic effect over an extended period of time. To maintain a constant level of the compound of the invention in the body, the compound of the invention can be released from the dosage form at a rate that will replace the amount of compound of the invention being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds. Controlled-release and sustained-release means for use according to the present invention may be selected from those known in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, each of which is incorporated herein by reference. Such dosage forms can be used to provide controlled- or sustained-release of one or more active ingredients using, for example, hydroxypropylmethyl cellulose, other polymer matrices, gels, permeable membranes, osmotic systems, multilayer coatings, microparticles, multiparticulates, liposomes, microspheres, or a combination thereof to provide the desired release profile in varying proportions. Suitable controlled- or sustained-release formulations known in the art, including those described herein, can be readily selected for use with the active ingredients of the invention in view of this disclosure. See also Goodson, “Dental Applications” (pp. 1 15-138) in Medical Applications of Controlled Release, Vol. 2, Applications and Evaluation, R. S. Langer and D. L. Wise eds., CRC Press (1984). Other controlled- or sustained-release systems that are discussed in the review by Langer, Science 249: 1527-1533 (1990) can be selected for use according to the present invention. In one embodiment, a pump can be used (Langer, Science 249: 1527-1533 (1990); Sefton, CRC Crit. Re Biomed. Eng. 74:201 (1987); Buchwald et al, Surgery 88:507 (1980); and Saudek et al, N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 25:61 (1983); Levy et al, Science 225: 190 (1985); During et al, Ann. Neurol. 25:351 (1989); and Howard et al, J. Neurosurg. 7i:105 (1989)). In yet another embodiment, a controlled- or sustained-release system can be placed in proximity of a target of a compound of the invention, e.g., the spinal column, brain, or gastrointestinal tract, thus requiring only a fraction of the systemic dose.

When in tablet or pill form, a pharmaceutical composition of the invention can be coated to delay disintegration and absorption in the gastrointestinal tract, thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

Pharmaceutical compositions of the invention include single unit dosage forms suitable for oral administration such as, but not limited to, tablets, capsules, gelcaps, and caplets that are adapted for controlled- or sustained-release.

### Methods of Making Compounds of the Invention

In one embodiment, the invention is a method of preparing a compound of formula I,

wherein A is:

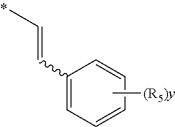

and B, R₁, R₂, R₃, R₄, R₅, R₆, x, and y are as defined above,

comprising reacting a compound of formula III

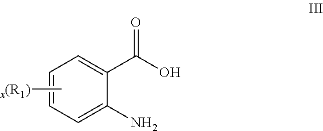

with acetic anhydride in the presence of a base to obtain a compound of formula IV

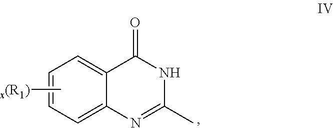

reacting the compound of formula IV with a compound of formula V,

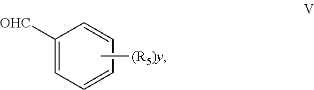

to obtain a compound of formula VI,

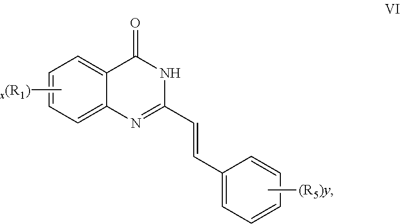

and reacting the compound of formula VI with a compound of formula VII

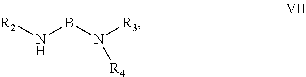

to obtain a compound of formula I.

In one embodiment, the invention is a method of preparing a compound of formula I, wherein A is:

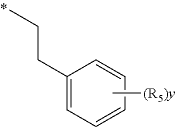

comprising reacting a compound of formula VI

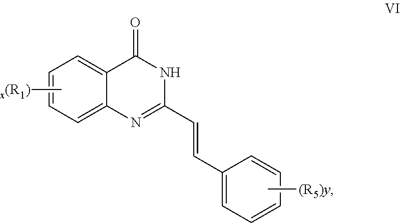

with hydrogen, optionally in the presence of a catalyst, under conditions sufficient to form a compound of formula I.

In some embodiments, the invention is a method of preparing a compound of formula II, wherein

A is:

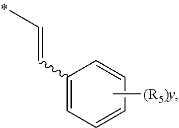

comprising

reacting a compound of formula VIII:

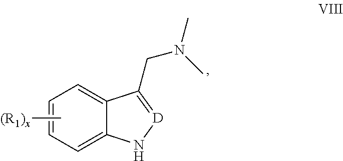

with a compound of formula V

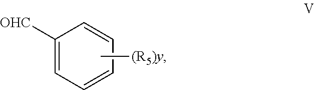

to obtain a compound of formula II.

In some embodiments, the invention is a method of preparing a compound of formula III, wherein

A is:

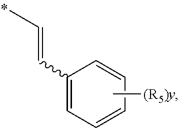

and D, R₁, R₅, x, and y are as defined above,

comprising

reacting a compound of formula VIII:

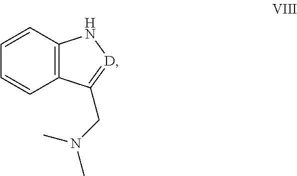

with a compound of formula V,

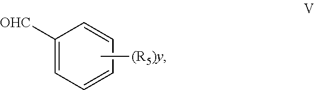

under conditions sufficient to obtain a compound of formula II.

In one embodiment, the invention is a method of preparing a compound of formula I, wherein A is:

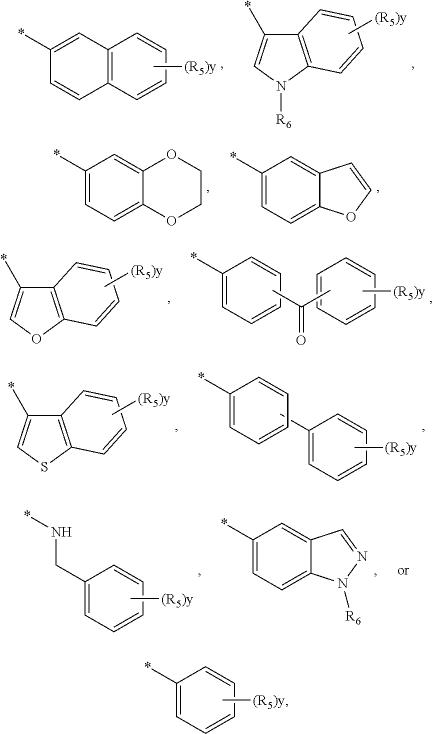

and B, R₁, R₂, R₃, R₄, R₅, R₆, x, and y are as defined above,

comprising reacting a compound of formula IX

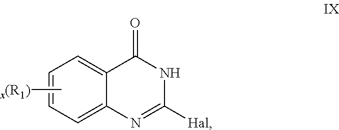

wherein Hal is IX, R₁ and x are as defined above,

with a compound of formula X,

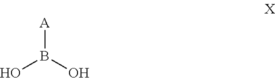

optionally in the presence of a catalyst under conditions sufficient to form a compound of formula I.

In some embodiments, the invention is a method of preparing a compound of formula III, wherein A is:

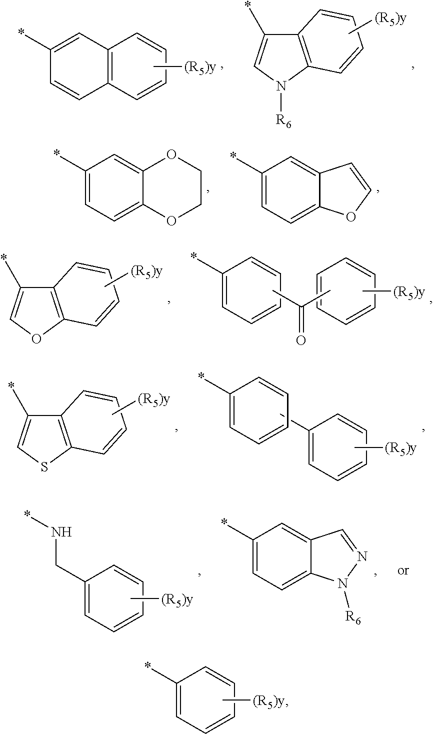

and R₁, R₅, R₆, x, and y are as defined above,

comprising reacting a compound of formula XI

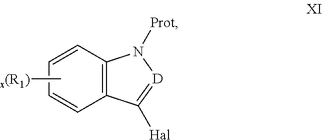

wherein Hal is halogen, Prot is a protecting group, D, R₁ and x are as defined above,

with a compound of formula X,

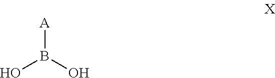

optionally in the presence of a catalyst under conditions sufficient to form a compound of formula III.

### Exemplary Compounds of the Invention

The following compounds of Tables 1-15 are exemplary compounds of the invention. These compound are/were made using the procedures, or analogous procedures, disclosed herein. Substitutions shown in each table entry number (“No.”) are used with the corresponding general structure for the table to depict a particular compound. For example, with regard to compound No. 1 of Table 1, the substitution pattern taken together with the general structure associated with Table 1 generates the following corresponding formula for compound No. 1:

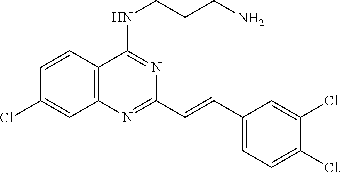 [SMILES: NCCCNc1nc(/C=C/c2ccc(Cl)c(Cl)c2)nc2cc(Cl)ccc12]

Tables 1, 2, and 3 depict compounds of Formula Ia:

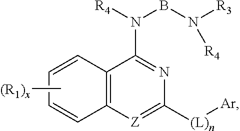

wherein Z is N.

TABLE 1

| No. | R₁ | R₂ | R₃ | R₄ | B | L (n = 1) | Ar |
|---|---|---|---|---|---|---|---|
| 1 | 7-Cl | H | H | H | (CH₂)₃ | trans-CH═CH | 3,4-(Cl)₂—Ph |
| 2 | 8-Cl | H | H | H | (CH₂)₃ | trans-CH═CH | 3,4-(Cl)₂—Ph |
| 3 | 5-Cl | H | H | H | (CH₂)₃ | trans-CH═CH | 3,4-(Cl)₂—Ph |
| 4 | 6-Cl | H | H | H | (CH₂)₃ | trans-CH═CH | 3,4-(Cl)₂—Ph |
| 5 | 5-CH₃ | H | H | H | (CH₂)₃ | trans-CH═CH | 3,4-(Cl)₂—Ph |
| 6 | 6-CH₃ | H | H | H | (CH₂)₃ | trans-CH═CH | 3,4-(Cl)₂—Ph |
| 7 | 7-CH₃ | H | H | H | (CH₂)₃ | trans-CH═CH | 3,4-(Cl)₂—Ph |
| 8 | 8-CH₃ | H | H | H | (CH₂)₃ | trans-CH═CH | 3,4-(Cl)₂—Ph |
| 9 | 5-ethyl | H | H | H | (CH₂)₃ | trans-CH═CH | 3,4-(Cl)₂—Ph |
| 10 | 6-ethyl | H | H | H | (CH₂)₃ | trans-CH═CH | 3,4-(Cl)₂—Ph |
| 11 | 7-ethyl | H | H | H | (CH₂)₃ | trans-CH═CH | 3,4-(Cl)₂—Ph |
| 12 | 8-ethyl | H | H | H | (CH₂)₃ | trans-CH═CH | 3,4-(Cl)₂—Ph |
| 13 | 5,6-(Cl)₂ | H | H | H | (CH₂)₃ | trans-CH═CH | 3,4-(Cl)₂—Ph |
| 14 | 6,7-(Cl)₂ | H | H | H | (CH₂)₃ | trans-CH═CH | 3,4-(Cl)₂—Ph |
| 15 | 7,8-(Cl)₂ | H | H | H | (CH₂)₃ | trans-CH═CH | 3,4-(Cl)₂—Ph |
| 16 | 5,7-(Cl)₂ | H | H | H | (CH₂)₃ | trans-CH═CH | 3,4-(Cl)₂—Ph |
| 17 | 6,8-(Cl)₂ | H | H | H | (CH₂)₃ | trans-CH═CH | 3,4-(Cl)₂—Ph |
| 18 | 5,8-(Cl)₂ | H | H | H | (CH₂)₃ | trans-CH═CH | 3,4-(Cl)₂—Ph |
| 19 | 5-F | H | H | H | (CH₂)₃ | trans-CH═CH | 3,4-(Cl)₂—Ph |
| 20 | 6-F | H | H | H | (CH₂)₃ | trans-CH═CH | 3,4-(Cl)₂—Ph |
| 21 | 7-F | H | H | H | (CH₂)₃ | trans-CH═CH | 3,4-(Cl)₂—Ph |
| 22 | 8-F | H | H | H | (CH₂)₃ | trans-CH═CH | 3,4-(Cl)₂—Ph |
| 23 | 5-Br | H | H | H | (CH₂)₃ | trans-CH═CH | 3,4-(Cl)₂—Ph |
| 24 | 6-Br | H | H | H | (CH₂)₃ | trans-CH═CH | 3,4-(Cl)₂—Ph |
| 25 | 7-Br | H | H | H | (CH₂)₃ | trans-CH═CH | 3,4-(Cl)₂—Ph |
| 26 | 8-Br | H | H | H | (CH₂)₃ | trans-CH═CH | 3,4-(Cl)₂—Ph |
| 27 | 5-CF₃ | H | H | H | (CH₂)₃ | trans-CH═CH | 3,4-(Cl)₂—Ph |
| 28 | 6-CF₃ | H | H | H | (CH₂)₃ | trans-CH═CH | 3,4-(Cl)₂—Ph |
| 29 | 7-CF₃ | H | H | H | (CH₂)₃ | trans-CH═CH | 3,4-(Cl)₂—Ph |
| 30 | 8-CF₃ | H | H | H | (CH₂)₃ | trans-CH═CH | 3,4-(Cl)₂—Ph |
| 31 | 7-Cl | CH₃ | H | H | (CH₂)₃ | trans-CH═CH | 3,4-(Cl)₂—Ph |
| 32 | 8-Cl | CH₃ | H | H | (CH₂)₃ | trans-CH═CH | 3,4-(Cl)₂—Ph |
| 33 | 5-Cl | CH₃ | H | H | (CH₂)₃ | trans-CH═CH | 3,4-(Cl)₂—Ph |
| 34 | 6-Cl | CH₃ | H | H | (CH₂)₃ | trans-CH═CH | 3,4-(Cl)₂—Ph |
| 35 | 5-CH₃ | CH₃ | H | H | (CH₂)₃ | trans-CH═CH | 3,4-(Cl)₂—Ph |
| 36 | 6-CH₃ | CH₃ | H | H | (CH₂)₃ | trans-CH═CH | 3,4-(Cl)₂—Ph |
| 37 | 7-CH₃ | CH₃ | H | H | (CH₂)₃ | trans-CH═CH | 3,4-(Cl)₂—Ph |
| 38 | 8-CH₃ | CH₃ | H | H | (CH₂)₃ | trans-CH═CH | 3,4-(Cl)₂—Ph |
| 39 | 5-ethyl | CH₃ | H | H | (CH₂)₃ | trans-CH═CH | 3,4-(Cl)₂—Ph |
| 40 | 6-ethyl | CH₃ | H | H | (CH₂)₃ | trans-CH═CH | 3,4-(Cl)₂—Ph |
| 41 | 7-ethyl | ethyl | H | H | (CH₂)₃ | trans-CH═CH | 3,4-(Cl)₂—Ph |
| 42 | 8-ethyl | CH₃ | H | H | (CH₂)₃ | trans-CH═CH | 3,4-(Cl)₂—Ph |
| 43 | 5,6-(Cl)₂ | CH₃ | H | H | (CH₂)₃ | trans-CH═CH | 3,4-(Cl)₂—Ph |
| 44 | 6,7-(Cl)₂ | CH₃ | H | H | (CH₂)₃ | trans-CH═CH | 3,4-(Cl)₂—Ph |
| 45 | 7,8-(Cl)₂ | CH₃ | H | H | (CH₂)₃ | trans-CH═CH | 3,4-(Cl)₂—Ph |
| 46 | 5,7-(Cl)₂ | CH₃ | H | H | (CH₂)₃ | trans-CH═CH | 3,4-(Cl)₂—Ph |
| 47 | 6,8-(Cl)₂ | CH₃ | H | H | (CH₂)₃ | trans-CH═CH | 3,4-(Cl)₂—Ph |
| 48 | 5,8-(Cl)₂ | CH₃ | H | H | (CH₂)₃ | trans-CH═CH | 3,4-(Cl)₂—Ph |
| 49 | 5-F | CH₃ | H | H | (CH₂)₃ | trans-CH═CH | 3,4-(Cl)₂—Ph |
| 50 | 6-F | CH₃ | H | H | (CH₂)₃ | trans-CH═CH | 3,4-(Cl)₂—Ph |
| 51 | 7-F | CH₃ | H | H | (CH₂)₃ | trans-CH═CH | 3,4-(Cl)₂—Ph |
| 52 | 8-F | CH₃ | H | H | (CH₂)₃ | trans-CH═CH | 3,4-(Cl)₂—Ph |
| 53 | 5-Br | CH₃ | H | H | (CH₂)₃ | trans-CH═CH | 3,4-(Cl)₂—Ph |
| 54 | 6-Br | CH₃ | H | H | (CH₂)₃ | trans-CH═CH | 3,4-(Cl)₂—Ph |
| 55 | 7-Br | CH₃ | H | H | (CH₂)₃ | trans-CH═CH | 3,4-(Cl)₂—Ph |
| 56 | 8-Br | CH₃ | H | H | (CH₂)₃ | trans-CH═CH | 3,4-(Cl)₂—Ph |
| 57 | 5-CF₃ | CH₃ | H | H | (CH₂)₃ | trans-CH═CH | 3,4-(Cl)₂—Ph |
| 58 | 6-CF₃ | CH₃ | H | H | (CH₂)₃ | trans-CH═CH | 3,4-(Cl)₂—Ph |
| 59 | 7-CF₃ | CH₃ | H | H | (CH₂)₃ | trans-CH═CH | 3,4-(Cl)₂—Ph |
| 60 | 8-CF₃ | CH₃ | H | H | (CH₂)₃ | trans-CH═CH | 3,4-(Cl)₂—Ph |
| 61 | 7-Cl | H | CH₃ | CH₃ | (CH₂)₃ | trans-CH═CH | 3,4-(Cl)₂—Ph |
| 62 | 8-Cl | H | CH₃ | CH₃ | (CH₂)₃ | trans-CH═CH | 3,4-(Cl)₂—Ph |
| 63 | 5-Cl | H | CH₃ | CH₃ | (CH₂)₃ | trans-CH═CH | 3,4-(Cl)₂—Ph |
| 64 | 6-Cl | H | CH₃ | CH₃ | (CH₂)₃ | trans-CH═CH | 3,4-(Cl)₂—Ph |
| 65 | 5-CH₃ | H | CH₃ | CH₃ | (CH₂)₃ | trans-CH═CH | 3,4-(Cl)₂—Ph |
| 66 | 6-CH₃ | H | CH₃ | CH₃ | (CH₂)₃ | trans-CH═CH | 3,4-(Cl)₂—Ph |
| 67 | 7-CH₃ | H | CH₃ | CH₃ | (CH₂)₃ | trans-CH═CH | 3,4-(Cl)₂—Ph |
| 68 | 8-CH₃ | H | CH₃ | CH₃ | (CH₂)₃ | trans-CH═CH | 3,4-(Cl)₂—Ph |
| 69 | 5-ethyl | H | CH₃ | CH₃ | (CH₂)₃ | trans-CH═CH | 3,4-(Cl)₂—Ph |
| 70 | 6-ethyl | H | CH₃ | CH₃ | (CH₂)₃ | trans-CH═CH | 3,4-(Cl)₂—Ph |
| 71 | 7-ethyl | H | CH₃ | CH₃ | (CH₂)₃ | trans-CH═CH | 3,4-(Cl)₂—Ph |
| 72 | 8-ethyl | H | CH₃ | CH₃ | (CH₂)₃ | trans-CH═CH | 3,4-(Cl)₂—Ph |
| 73 | 5,6-(Cl)₂ | H | CH₃ | CH₃ | (CH₂)₃ | trans-CH═CH | 3,4-(Cl)₂—Ph |
| 74 | 6,7-(Cl)₂ | H | CH₃ | CH₃ | (CH₂)₃ | trans-CH═CH | 3,4-(Cl)₂—Ph |
| 75 | 7,8-(Cl)₂ | H | CH₃ | CH₃ | (CH₂)₃ | trans-CH═CH | 3,4-(Cl)₂—Ph |
| 76 | 5,7-(Cl)₂ | H | CH₃ | CH₃ | (CH₂)₃ | trans-CH═CH | 3,4-(Cl)₂—Ph |
| 77 | 6,8-(Cl)₂ | H | CH₃ | CH₃ | (CH₂)₃ | trans-CH═CH | 3,4-(Cl)₂—Ph |
| 78 | 5,8-(Cl)₂ | H | CH₃ | CH₃ | (CH₂)₃ | trans-CH═CH | 3,4-(Cl)₂—Ph |
| 79 | 5-F | H | CH₃ | CH₃ | (CH₂)₃ | trans-CH═CH | 3,4-(Cl)₂—Ph |
| 80 | 6-F | H | CH₃ | CH₃ | (CH₂)₃ | trans-CH═CH | 3,4-(Cl)₂—Ph |
| 81 | 7-F | H | CH₃ | CH₃ | (CH₂)₃ | trans-CH═CH | 3,4-(Cl)₂—Ph |
| 82 | 8-F | H | CH₃ | CH₃ | (CH₂)₃ | trans-CH═CH | 3,4-(Cl)₂—Ph |
| 83 | 5-Br | H | CH₃ | CH₃ | (CH₂)₃ | trans-CH═CH | 3,4-(Cl)₂—Ph |
| 84 | 6-Br | H | CH₃ | CH₃ | (CH₂)₃ | trans-CH═CH | 3,4-(Cl)₂—Ph |
| 85 | 7-Br | H | CH₃ | CH₃ | (CH₂)₃ | trans-CH═CH | 3,4-(Cl)₂—Ph |
| 86 | 8-Br | H | CH₃ | CH₃ | (CH₂)₃ | trans-CH═CH | 3,4-(Cl)₂—Ph |
| 87 | 5-CF₃ | H | CH₃ | CH₃ | (CH₂)₃ | trans-CH═CH | 3,4-(Cl)₂—Ph |
| 88 | 6-CF₃ | H | CH₃ | CH₃ | (CH₂)₃ | trans-CH═CH | 3,4-(Cl)₂—Ph |
| 89 | 7-CF₃ | H | CH₃ | CH₃ | (CH₂)₃ | trans-CH═CH | 3,4-(Cl)₂—Ph |
| 90 | 8-CF₃ | H | CH₃ | CH₃ | (CH₂)₃ | trans-CH═CH | 3,4-(Cl)₂—Ph |
| 91 | 7-Cl | H | ethyl | ethyl | (CH₂)₃ | trans-CH═CH | 3,4-(Cl)₂—Ph |
| 92 | 8-Cl | H | ethyl | ethyl | (CH₂)₃ | trans-CH═CH | 3,4-(Cl)₂—Ph |
| 93 | 5-Cl | H | ethyl | ethyl | (CH₂)₃ | trans-CH═CH | 3,4-(Cl)₂—Ph |
| 94 | 6-Cl | H | ethyl | ethyl | (CH₂)₃ | trans-CH═CH | 3,4-(Cl)₂—Ph |
| 95 | 5-CH₃ | H | ethyl | ethyl | (CH₂)₃ | trans-CH═CH | 3,4-(Cl)₂—Ph |
| 96 | 6-CH₃ | H | ethyl | ethyl | (CH₂)₃ | trans-CH═CH | 3,4-(Cl)₂—Ph |
| 97 | 7-CH₃ | H | ethyl | ethyl | (CH₂)₃ | trans-CH═CH | 3,4-(Cl)₂—Ph |
| 98 | 8-CH₃ | H | ethyl | ethyl | (CH₂)₃ | trans-CH═CH | 3,4-(Cl)₂—Ph |
| 99 | 5-ethyl | H | ethyl | ethyl | (CH₂)₃ | trans-CH═CH | 3,4-(Cl)₂—Ph |
| 100 | 6-ethyl | H | ethyl | ethyl | (CH₂)₃ | trans-CH═CH | 3,4-(Cl)₂—Ph |
| 101 | 7-ethyl | H | ethyl | ethyl | (CH₂)₃ | trans-CH═CH | 3,4-(Cl)₂—Ph |
| 102 | 8-ethyl | H | ethyl | ethyl | (CH₂)₃ | trans-CH═CH | 3,4-(Cl)₂—Ph |
| 103 | 5,6-(Cl)₂ | H | ethyl | ethyl | (CH₂)₃ | trans-CH═CH | 3,4-(Cl)₂—Ph |
| 104 | 6,7-(Cl)₂ | H | ethyl | ethyl | (CH₂)₃ | trans-CH═CH | 3,4-(Cl)₂—Ph |
| 105 | 7,8-(Cl)₂ | H | ethyl | ethyl | (CH₂)₃ | trans-CH═CH | 3,4-(Cl)₂—Ph |
| 106 | 5,7-(Cl)₂ | H | ethyl | ethyl | (CH₂)₃ | trans-CH═CH | 3,4-(Cl)₂—Ph |
| 107 | 6,8-(Cl)₂ | H | ethyl | ethyl | (CH₂)₃ | trans-CH═CH | 3,4-(Cl)₂—Ph |
| 108 | 5,8-(Cl)₂ | H | ethyl | ethyl | (CH₂)₃ | trans-CH═CH | 3,4-(Cl)₂—Ph |
| 109 | 5-F | H | ethyl | ethyl | (CH₂)₃ | trans-CH═CH | 3,4-(Cl)₂—Ph |
| 110 | 6-F | H | ethyl | ethyl | (CH₂)₃ | trans-CH═CH | 3,4-(Cl)₂—Ph |
| 111 | 7-F | H | ethyl | ethyl | (CH₂)₃ | trans-CH═CH | 3,4-(Cl)₂—Ph |
| 112 | 8-F | H | ethyl | ethyl | (CH₂)₃ | trans-CH═CH | 3,4-(Cl)₂—Ph |
| 113 | 5-Br | H | ethyl | ethyl | (CH₂)₃ | trans-CH═CH | 3,4-(Cl)₂—Ph |
| 114 | 6-Br | H | ethyl | ethyl | (CH₂)₃ | trans-CH═CH | 3,4-(Cl)₂—Ph |
| 115 | 7-Br | H | ethyl | ethyl | (CH₂)₃ | trans-CH═CH | 3,4-(Cl)₂—Ph |
| 116 | 8-Br | H | ethyl | ethyl | (CH₂)₃ | trans-CH═CH | 3,4-(Cl)₂—Ph |
| 117 | 5-CF₃ | H | ethyl | ethyl | (CH₂)₃ | trans-CH═CH | 3,4-(Cl)₂—Ph |
| 118 | 6-CF₃ | H | ethyl | ethyl | (CH₂)₃ | trans-CH═CH | 3,4-(Cl)₂—Ph |
| 119 | 7-CF₃ | H | ethyl | ethyl | (CH₂)₃ | trans-CH═CH | 3,4-(Cl)₂—Ph |
| 120 | 8-CF₃ | H | ethyl | ethyl | (CH₂)₃ | trans-CH═CH | 3,4-(Cl)₂—Ph |
| 121 | 7-Cl | H | CH₃ | CH₃ | (CH₂)₃ | trans-CH═CH | 3,5-(Cl)₂—Ph |
| 122 | 8-Cl | H | CH₃ | CH₃ | (CH₂)₃ | trans-CH═CH | 3,5-(Cl)₂—Ph |
| 123 | 5-Cl | H | CH₃ | CH₃ | (CH₂)₃ | trans-CH═CH | 3,5-(Cl)₂—Ph |
| 124 | 6-Cl | H | CH₃ | CH₃ | (CH₂)₃ | trans-CH═CH | 3,5-(Cl)₂—Ph |
| 125 | 5-CH₃ | H | CH₃ | CH₃ | (CH₂)₃ | trans-CH═CH | 3,5-(Cl)₂—Ph |
| 126 | 6-CH₃ | H | CH₃ | CH₃ | (CH₂)₃ | trans-CH═CH | 3,5-(Cl)₂—Ph |
| 127 | 7-CH₃ | H | CH₃ | CH₃ | (CH₂)₃ | trans-CH═CH | 3,5-(Cl)₂—Ph |
| 128 | 8-CH₃ | H | CH₃ | CH₃ | (CH₂)₃ | trans-CH═CH | 3,5-(Cl)₂—Ph |
| 129 | 5-ethyl | H | CH₃ | CH₃ | (CH₂)₃ | trans-CH═CH | 3,5-(Cl)₂—Ph |
| 130 | 6-ethyl | H | CH₃ | CH₃ | (CH₂)₃ | trans-CH═CH | 3,5-(Cl)₂—Ph |
| 131 | 7-ethyl | H | CH₃ | CH₃ | (CH₂)₃ | trans-CH═CH | 3,5-(Cl)₂—Ph |
| 132 | 8-ethyl | H | CH₃ | CH₃ | (CH₂)₃ | trans-CH═CH | 3,5-(Cl)₂—Ph |
| 133 | 5,6-(Cl)₂ | H | CH₃ | CH₃ | (CH₂)₃ | trans-CH═CH | 3,5-(Cl)₂—Ph |
| 134 | 6,7-(Cl)₂ | H | CH₃ | CH₃ | (CH₂)₃ | trans-CH═CH | 3,5-(Cl)₂—Ph |
| 135 | 7,8-(Cl)₂ | H | CH₃ | CH₃ | (CH₂)₃ | trans-CH═CH | 3,5-(Cl)₂—Ph |
| 136 | 5,7-(Cl)₂ | H | CH₃ | CH₃ | (CH₂)₃ | trans-CH═CH | 3,5-(Cl)₂—Ph |
| 137 | 6,8-(Cl)₂ | H | CH₃ | CH₃ | (CH₂)₃ | trans-CH═CH | 3,5-(Cl)₂—Ph |
| 138 | 5,8-(Cl)₂ | H | CH₃ | CH₃ | (CH₂)₃ | trans-CH═CH | 3,5-(Cl)₂—Ph |
| 139 | 5-F | H | CH₃ | CH₃ | (CH₂)₃ | trans-CH═CH | 3,5-(Cl)₂—Ph |
| 140 | 6-F | H | CH₃ | CH₃ | (CH₂)₃ | trans-CH═CH | 3,5-(Cl)₂—Ph |
| 141 | 7-F | H | CH₃ | CH₃ | (CH₂)₃ | trans-CH═CH | 3,5-(Cl)₂—Ph |
| 142 | 8-F | H | CH₃ | CH₃ | (CH₂)₃ | trans-CH═CH | 3,5-(Cl)₂—Ph |
| 143 | 5-Br | H | CH₃ | CH₃ | (CH₂)₃ | trans-CH═CH | 3,5-(Cl)₂—Ph |
| 144 | 6-Br | H | CH₃ | CH₃ | (CH₂)₃ | trans-CH═CH | 3,5-(Cl)₂—Ph |
| 145 | 7-Br | H | CH₃ | CH₃ | (CH₂)₃ | trans-CH═CH | 3,5-(Cl)₂—Ph |
| 146 | 8-Br | H | CH₃ | CH₃ | (CH₂)₃ | trans-CH═CH | 3,5-(Cl)₂—Ph |
| 147 | 5-CF₃ | H | CH₃ | CH₃ | (CH₂)₃ | trans-CH═CH | 3,5-(Cl)₂—Ph |
| 148 | 6-CF₃ | H | CH₃ | CH₃ | (CH₂)₃ | trans-CH═CH | 3,5-(Cl)₂—Ph |
| 149 | 7-CF₃ | H | CH₃ | CH₃ | (CH₂)₃ | trans-CH═CH | 3,5-(Cl)₂—Ph |
| 150 | 8-CF₃ | H | CH₃ | CH₃ | (CH₂)₃ | trans-CH═CH | 3,5-(Cl)₂—Ph |
| 151 | 7-Cl | H | ethyl | ethyl | (CH₂)₃ | trans-CH═CH | 3,5-(Cl)₂—Ph |
| 152 | 8-Cl | H | ethyl | ethyl | (CH₂)₃ | trans-CH═CH | 3,5-(Cl)₂—Ph |
| 153 | 5-Cl | H | ethyl | ethyl | (CH₂)₃ | trans-CH═CH | 3,5-(Cl)₂—Ph |
| 154 | 6-Cl | H | ethyl | ethyl | (CH₂)₃ | trans-CH═CH | 3,5-(Cl)₂—Ph |
| 155 | 5-CH₃ | H | ethyl | ethyl | (CH₂)₃ | trans-CH═CH | 3,5-(Cl)₂—Ph |
| 156 | 6-CH₃ | H | ethyl | ethyl | (CH₂)₃ | trans-CH═CH | 3,5-(Cl)₂—Ph |
| 157 | 7-CH₃ | H | ethyl | ethyl | (CH₂)₃ | trans-CH═CH | 3,5-(Cl)₂—Ph |
| 158 | 8-CH₃ | H | ethyl | ethyl | (CH₂)₃ | trans-CH═CH | 3,5-(Cl)₂—Ph |
| 159 | 5-ethyl | H | ethyl | ethyl | (CH₂)₃ | trans-CH═CH | 3,5-(Cl)₂—Ph |
| 160 | 6-ethyl | H | ethyl | ethyl | (CH₂)₃ | trans-CH═CH | 3,5-(Cl)₂—Ph |
| 161 | 7-ethyl | H | ethyl | ethyl | (CH₂)₃ | trans-CH═CH | 3,5-(Cl)₂—Ph |
| 162 | 8-ethyl | H | ethyl | ethyl | (CH₂)₃ | trans-CH═CH | 3,5-(Cl)₂—Ph |
| 163 | 5,6-(Cl)₂ | H | ethyl | ethyl | (CH₂)₃ | trans-CH═CH | 3,5-(Cl)₂—Ph |
| 164 | 6,7-(Cl)₂ | H | ethyl | ethyl | (CH₂)₃ | trans-CH═CH | 3,5-(Cl)₂—Ph |
| 165 | 7,8-(Cl)₂ | H | ethyl | ethyl | (CH₂)₃ | trans-CH═CH | 3,5-(Cl)₂—Ph |
| 166 | 5,7-(Cl)₂ | H | ethyl | ethyl | (CH₂)₃ | trans-CH═CH | 3,5-(Cl)₂—Ph |
| 167 | 6,8-(Cl)₂ | H | ethyl | ethyl | (CH₂)₃ | trans-CH═CH | 3,5-(Cl)₂—Ph |
| 168 | 5,8-(Cl)₂ | H | ethyl | ethyl | (CH₂)₃ | trans-CH═CH | 3,5-(Cl)₂—Ph |
| 169 | 5-F | H | ethyl | ethyl | (CH₂)₃ | trans-CH═CH | 3,5-(Cl)₂—Ph |
| 170 | 6-F | H | ethyl | ethyl | (CH₂)₃ | trans-CH═CH | 3,5-(Cl)₂—Ph |
| 171 | 7-F | H | ethyl | ethyl | (CH₂)₃ | trans-CH═CH | 3,5-(Cl)₂—Ph |
| 172 | 8-F | H | ethyl | ethyl | (CH₂)₃ | trans-CH═CH | 3,5-(Cl)₂—Ph |
| 173 | 5-Br | H | ethyl | ethyl | (CH₂)₃ | trans-CH═CH | 3,5-(Cl)₂—Ph |
| 174 | 6-Br | H | ethyl | ethyl | (CH₂)₃ | trans-CH═CH | 3,5-(Cl)₂—Ph |
| 175 | 7-Br | H | ethyl | ethyl | (CH₂)₃ | trans-CH═CH | 3,5-(Cl)₂—Ph |
| 176 | 8-Br | H | ethyl | ethyl | (CH₂)₃ | trans-CH═CH | 3,5-(Cl)₂—Ph |
| 177 | 5-CF₃ | H | ethyl | ethyl | (CH₂)₃ | trans-CH═CH | 3,5-(Cl)₂—Ph |
| 178 | 6-CF₃ | H | ethyl | ethyl | (CH₂)₃ | trans-CH═CH | 3,5-(Cl)₂—Ph |
| 179 | 7-CF₃ | H | ethyl | ethyl | (CH₂)₃ | trans-CH═CH | 3,5-(Cl)₂—Ph |
| 180 | 8-CF₃ | H | ethyl | ethyl | (CH₂)₃ | trans-CH═CH | 3,5-(Cl)₂—Ph |
| 181 | 7-Cl | H | CH₃ | CH₃ | (CH₂)₃ | trans-CH═CH | 3,4-(F)₂—Ph |
| 182 | 8-Cl | H | CH₃ | CH₃ | (CH₂)₃ | trans-CH═CH | 3,4-(F)₂—Ph |
| 183 | 5-Cl | H | CH₃ | CH₃ | (CH₂)₃ | trans-CH═CH | 3,4-(F)₂—Ph |
| 184 | 6-Cl | H | CH₃ | CH₃ | (CH₂)₃ | trans-CH═CH | 3,4-(F)₂—Ph |
| 185 | 5-CH₃ | H | CH₃ | CH₃ | (CH₂)₃ | trans-CH═CH | 3,4-(F)₂—Ph |
| 186 | 6-CH₃ | H | CH₃ | CH₃ | (CH₂)₃ | trans-CH═CH | 3,4-(F)₂—Ph |
| 187 | 7-CH₃ | H | CH₃ | CH₃ | (CH₂)₃ | trans-CH═CH | 3,4-(F)₂—Ph |
| 188 | 8-CH₃ | H | CH₃ | CH₃ | (CH₂)₃ | trans-CH═CH | 3,4-(F)₂—Ph |
| 189 | 5-ethyl | H | CH₃ | CH₃ | (CH₂)₃ | trans-CH═CH | 3,4-(F)₂—Ph |
| 190 | 6-ethyl | H | CH₃ | CH₃ | (CH₂)₃ | trans-CH═CH | 3,4-(F)₂—Ph |
| 191 | 7-ethyl | H | CH₃ | CH₃ | (CH₂)₃ | trans-CH═CH | 3,4-(F)₂—Ph |
| 192 | 8-ethyl | H | CH₃ | CH₃ | (CH₂)₃ | trans-CH═CH | 3,4-(F)₂—Ph |
| 193 | 5,6-(Cl)₂ | H | CH₃ | CH₃ | (CH₂)₃ | trans-CH═CH | 3,4-(F)₂—Ph |
| 194 | 6,7-(Cl)₂ | H | CH₃ | CH₃ | (CH₂)₃ | trans-CH═CH | 3,4-(F)₂—Ph |
| 195 | 7,8-(Cl)₂ | H | CH₃ | CH₃ | (CH₂)₃ | trans-CH═CH | 3,4-(F)₂—Ph |
| 196 | 5,7-(Cl)₂ | H | CH₃ | CH₃ | (CH₂)₃ | trans-CH═CH | 3,4-(F)₂—Ph |
| 197 | 6,8-(Cl)₂ | H | CH₃ | CH₃ | (CH₂)₃ | trans-CH═CH | 3,4-(F)₂—Ph |
| 198 | 5,8-(Cl)₂ | H | CH₃ | CH₃ | (CH₂)₃ | trans-CH═CH | 3,4-(F)₂—Ph |
| 199 | 5-F | H | CH₃ | CH₃ | (CH₂)₃ | trans-CH═CH | 3,4-(F)₂—Ph |
| 200 | 6-F | H | CH₃ | CH₃ | (CH₂)₃ | trans-CH═CH | 3,4-(F)₂—Ph |
| 201 | 7-F | H | CH₃ | CH₃ | (CH₂)₃ | trans-CH═CH | 3,4-(F)₂—Ph |
| 202 | 8-F | H | CH₃ | CH₃ | (CH₂)₃ | trans-CH═CH | 3,4-(F)₂—Ph |
| 203 | 5-Br | H | CH₃ | CH₃ | (CH₂)₃ | trans-CH═CH | 3,4-(F)₂—Ph |
| 204 | 6-Br | H | CH₃ | CH₃ | (CH₂)₃ | trans-CH═CH | 3,4-(F)₂—Ph |
| 205 | 7-Br | H | CH₃ | CH₃ | (CH₂)₃ | trans-CH═CH | 3,4-(F)₂—Ph |
| 206 | 8-Br | H | CH₃ | CH₃ | (CH₂)₃ | trans-CH═CH | 3,4-(F)₂—Ph |
| 207 | 5-CF₃ | H | CH₃ | CH₃ | (CH₂)₃ | trans-CH═CH | 3,4-(F)₂—Ph |
| 208 | 6-CF₃ | H | CH₃ | CH₃ | (CH₂)₃ | trans-CH═CH | 3,4-(F)₂—Ph |
| 209 | 7-CF₃ | H | CH₃ | CH₃ | (CH₂)₃ | trans-CH═CH | 3,4-(F)₂—Ph |
| 210 | 8-CF₃ | H | CH₃ | CH₃ | (CH₂)₃ | trans-CH═CH | 3,4-(F)₂—Ph |
| 211 | 7-Cl | H | ethyl | ethyl | (CH₂)₃ | trans-CH═CH | 3,4-(F)₂—Ph |
| 212 | 8-Cl | H | ethyl | ethyl | (CH₂)₃ | trans-CH═CH | 3,4-(F)₂—Ph |
| 213 | 5-Cl | H | ethyl | ethyl | (CH₂)₃ | trans-CH═CH | 3,4-(F)₂—Ph |
| 214 | 6-Cl | H | ethyl | ethyl | (CH₂)₃ | trans-CH═CH | 3,4-(F)₂—Ph |
| 215 | 5-CH₃ | H | ethyl | ethyl | (CH₂)₃ | trans-CH═CH | 3,4-(F)₂—Ph |
| 216 | 6-CH₃ | H | ethyl | ethyl | (CH₂)₃ | trans-CH═CH | 3,4-(F)₂—Ph |
| 217 | 7-CH₃ | H | ethyl | ethyl | (CH₂)₃ | trans-CH═CH | 3,4-(F)₂—Ph |
| 218 | 8-CH₃ | H | ethyl | ethyl | (CH₂)₃ | trans-CH═CH | 3,4-(F)₂—Ph |
| 219 | 5-ethyl | H | ethyl | ethyl | (CH₂)₃ | trans-CH═CH | 3,4-(F)₂—Ph |
| 220 | 6-ethyl | H | ethyl | ethyl | (CH₂)₃ | trans-CH═CH | 3,4-(F)₂—Ph |
| 221 | 7-ethyl | H | ethyl | ethyl | (CH₂)₃ | trans-CH═CH | 3,4-(F)₂—Ph |
| 222 | 8-ethyl | H | ethyl | ethyl | (CH₂)₃ | trans-CH═CH | 3,4-(F)₂—Ph |
| 223 | 5,6-(Cl)₂ | H | ethyl | ethyl | (CH₂)₃ | trans-CH═CH | 3,4-(F)₂—Ph |
| 224 | 6,7-(Cl)₂ | H | ethyl | ethyl | (CH₂)₃ | trans-CH═CH | 3,4-(F)₂—Ph |
| 225 | 7,8-(Cl)₂ | H | ethyl | ethyl | (CH₂)₃ | trans-CH═CH | 3,4-(F)₂—Ph |
| 226 | 5,7-(Cl)₂ | H | ethyl | ethyl | (CH₂)₃ | trans-CH═CH | 3,4-(F)₂—Ph |
| 227 | 6,8-(Cl)₂ | H | ethyl | ethyl | (CH₂)₃ | trans-CH═CH | 3,4-(F)₂—Ph |
| 228 | 5,8-(Cl)₂ | H | ethyl | ethyl | (CH₂)₃ | trans-CH═CH | 3,4-(F)₂—Ph |
| 229 | 5-F | H | ethyl | ethyl | (CH₂)₃ | trans-CH═CH | 3,4-(F)₂—Ph |
| 230 | 6-F | H | ethyl | ethyl | (CH₂)₃ | trans-CH═CH | 3,4-(F)₂—Ph |
| 231 | 7-F | H | ethyl | ethyl | (CH₂)₃ | trans-CH═CH | 3,4-(F)₂—Ph |
| 232 | 8-F | H | ethyl | ethyl | (CH₂)₃ | trans-CH═CH | 3,4-(F)₂—Ph |
| 233 | 5-Br | H | ethyl | ethyl | (CH₂)₃ | trans-CH═CH | 3,4-(F)₂—Ph |
| 234 | 6-Br | H | ethyl | ethyl | (CH₂)₃ | trans-CH═CH | 3,4-(F)₂—Ph |
| 235 | 7-Br | H | ethyl | ethyl | (CH₂)₃ | trans-CH═CH | 3,4-(F)₂—Ph |
| 236 | 8-Br | H | ethyl | ethyl | (CH₂)₃ | trans-CH═CH | 3,4-(F)₂—Ph |
| 237 | 5-CF₃ | H | ethyl | ethyl | (CH₂)₃ | trans-CH═CH | 3,4-(F)₂—Ph |
| 238 | 6-CF₃ | H | ethyl | ethyl | (CH₂)₃ | trans-CH═CH | 3,4-(F)₂—Ph |
| 239 | 7-CF₃ | H | ethyl | ethyl | (CH₂)₃ | trans-CH═CH | 3,4-(F)₂—Ph |
| 240 | 8-CF₃ | H | ethyl | ethyl | (CH₂)₃ | trans-CH═CH | 3,4-(F)₂—Ph |
| 241 | 7-Cl | H | CH₃ | CH₃ | (CH₂)₃ | trans-CH═CH | 2,4,6-(Cl)₃—Ph |
| 242 | 8-Cl | H | CH₃ | CH₃ | (CH₂)₃ | trans-CH═CH | 2,4,6-(Cl)₃—Ph |
| 243 | 5-Cl | H | CH₃ | CH₃ | (CH₂)₃ | trans-CH═CH | 2,4,6-(Cl)₃—Ph |
| 244 | 6-Cl | H | CH₃ | CH₃ | (CH₂)₃ | trans-CH═CH | 2,4,6-(Cl)₃—Ph |
| 245 | 5-CH₃ | H | CH₃ | CH₃ | (CH₂)₃ | trans-CH═CH | 2,4,6-(Cl)₃—Ph |
| 246 | 6-CH₃ | H | CH₃ | CH₃ | (CH₂)₃ | trans-CH═CH | 2,4,6-(Cl)₃—Ph |
| 247 | 7-CH₃ | H | CH₃ | CH₃ | (CH₂)₃ | trans-CH═CH | 2,4,6-(Cl)₃—Ph |
| 248 | 8-CH₃ | H | CH₃ | CH₃ | (CH₂)₃ | trans-CH═CH | 2,4,6-(Cl)₃—Ph |
| 249 | 5-ethyl | H | CH₃ | CH₃ | (CH₂)₃ | trans-CH═CH | 2,4,6-(Cl)₃—Ph |
| 250 | 6-ethyl | H | CH₃ | CH₃ | (CH₂)₃ | trans-CH═CH | 2,4,6-(Cl)₃—Ph |
| 251 | 7-ethyl | H | CH₃ | CH₃ | (CH₂)₃ | trans-CH═CH | 2,4,6-(Cl)₃—Ph |
| 252 | 8-ethyl | H | CH₃ | CH₃ | (CH₂)₃ | trans-CH═CH | 2,4,6-(Cl)₃—Ph |
| 253 | 5,6-(Cl)₂ | H | CH₃ | CH₃ | (CH₂)₃ | trans-CH═CH | 2,4,6-(Cl)₃—Ph |
| 254 | 6,7-(Cl)₂ | H | CH₃ | CH₃ | (CH₂)₃ | trans-CH═CH | 2,4,6-(Cl)₃—Ph |
| 255 | 7,8-(Cl)₂ | H | CH₃ | CH₃ | (CH₂)₃ | trans-CH═CH | 2,4,6-(Cl)₃—Ph |
| 256 | 5,7-(Cl)₂ | H | CH₃ | CH₃ | (CH₂)₃ | trans-CH═CH | 2,4,6-(Cl)₃—Ph |
| 257 | 6,8-(Cl)₂ | H | CH₃ | CH₃ | (CH₂)₃ | trans-CH═CH | 2,4,6-(Cl)₃—Ph |
| 258 | 5,8-(Cl)₂ | H | CH₃ | CH₃ | (CH₂)₃ | trans-CH═CH | 2,4,6-(Cl)₃—Ph |
| 259 | 5-F | H | CH₃ | CH₃ | (CH₂)₃ | trans-CH═CH | 2,4,6-(Cl)₃—Ph |
| 260 | 6-F | H | CH₃ | CH₃ | (CH₂)₃ | trans-CH═CH | 2,4,6-(Cl)₃—Ph |
| 261 | 7-F | H | CH₃ | CH₃ | (CH₂)₃ | trans-CH═CH | 2,4,6-(Cl)₃—Ph |
| 262 | 8-F | H | CH₃ | CH₃ | (CH₂)₃ | trans-CH═CH | 2,4,6-(Cl)₃—Ph |
| 263 | 5-Br | H | CH₃ | CH₃ | (CH₂)₃ | trans-CH═CH | 2,4,6-(Cl)₃—Ph |
| 264 | 6-Br | H | CH₃ | CH₃ | (CH₂)₃ | trans-CH═CH | 2,4,6-(Cl)₃—Ph |
| 265 | 7-Br | H | CH₃ | CH₃ | (CH₂)₃ | trans-CH═CH | 2,4,6-(Cl)₃—Ph |
| 266 | 8-Br | H | CH₃ | CH₃ | (CH₂)₃ | trans-CH═CH | 2,4,6-(Cl)₃—Ph |
| 267 | 5-CF₃ | H | CH₃ | CH₃ | (CH₂)₃ | trans-CH═CH | 2,4,6-(Cl)₃—Ph |
| 268 | 6-CF₃ | H | CH₃ | CH₃ | (CH₂)₃ | trans-CH═CH | 2,4,6-(Cl)₃—Ph |
| 269 | 7-CF₃ | H | CH₃ | CH₃ | (CH₂)₃ | trans-CH═CH | 2,4,6-(Cl)₃—Ph |
| 270 | 8-CF₃ | H | CH₃ | CH₃ | (CH₂)₃ | trans-CH═CH | 2,4,6-(Cl)₃—Ph |
| 271 | 7-Cl | H | ethyl | ethyl | (CH₂)₃ | trans-CH═CH | 2,4,6-(Cl)₃—Ph |
| 272 | 8-Cl | H | ethyl | ethyl | (CH₂)₃ | trans-CH═CH | 2,4,6-(Cl)₃—Ph |
| 273 | 5-Cl | H | ethyl | ethyl | (CH₂)₃ | trans-CH═CH | 2,4,6-(Cl)₃—Ph |
| 274 | 6-Cl | H | ethyl | ethyl | (CH₂)₃ | trans-CH═CH | 2,4,6-(Cl)₃—Ph |
| 275 | 5-CH₃ | H | ethyl | ethyl | (CH₂)₃ | trans-CH═CH | 2,4,6-(Cl)₃—Ph |
| 276 | 6-CH₃ | H | ethyl | ethyl | (CH₂)₃ | trans-CH═CH | 2,4,6-(Cl)₃—Ph |
| 277 | 7-CH₃ | H | ethyl | ethyl | (CH₂)₃ | trans-CH═CH | 2,4,6-(Cl)₃—Ph |
| 278 | 8-CH₃ | H | ethyl | ethyl | (CH₂)₃ | trans-CH═CH | 2,4,6-(Cl)₃—Ph |
| 279 | 5-ethyl | H | ethyl | ethyl | (CH₂)₃ | trans-CH═CH | 2,4,6-(Cl)₃—Ph |
| 280 | 6-ethyl | H | ethyl | ethyl | (CH₂)₃ | trans-CH═CH | 2,4,6-(Cl)₃—Ph |
| 281 | 7-ethyl | H | ethyl | ethyl | (CH₂)₃ | trans-CH═CH | 2,4,6-(Cl)₃—Ph |
| 282 | 8-ethyl | H | ethyl | ethyl | (CH₂)₃ | trans-CH═CH | 2,4,6-(Cl)₃—Ph |
| 283 | 5,6-(Cl)₂ | H | ethyl | ethyl | (CH₂)₃ | trans-CH═CH | 2,4,6-(Cl)₃—Ph |
| 284 | 6,7-(Cl)₂ | H | ethyl | ethyl | (CH₂)₃ | trans-CH═CH | 2,4,6-(Cl)₃—Ph |
| 285 | 7,8-(Cl)₂ | H | ethyl | ethyl | (CH₂)₃ | trans-CH═CH | 2,4,6-(Cl)₃—Ph |
| 286 | 5,7-(Cl)₂ | H | ethyl | ethyl | (CH₂)₃ | trans-CH═CH | 2,4,6-(Cl)₃—Ph |
| 287 | 6,8-(Cl)₂ | H | ethyl | ethyl | (CH₂)₃ | trans-CH═CH | 2,4,6-(Cl)₃—Ph |
| 288 | 5,8-(Cl)₂ | H | ethyl | ethyl | (CH₂)₃ | trans-CH═CH | 2,4,6-(Cl)₃—Ph |
| 289 | 5-F | H | ethyl | ethyl | (CH₂)₃ | trans-CH═CH | 2,4,6-(Cl)₃—Ph |
| 290 | 6-F | H | ethyl | ethyl | (CH₂)₃ | trans-CH═CH | 2,4,6-(Cl)₃—Ph |
| 291 | 7-F | H | ethyl | ethyl | (CH₂)₃ | trans-CH═CH | 2,4,6-(Cl)₃—Ph |
| 292 | 8-F | H | ethyl | ethyl | (CH₂)₃ | trans-CH═CH | 2,4,6-(Cl)₃—Ph |
| 293 | 5-Br | H | ethyl | ethyl | (CH₂)₃ | trans-CH═CH | 2,4,6-(Cl)₃—Ph |
| 294 | 6-Br | H | ethyl | ethyl | (CH₂)₃ | trans-CH═CH | 2,4,6-(Cl)₃—Ph |
| 295 | 7-Br | H | ethyl | ethyl | (CH₂)₃ | trans-CH═CH | 2,4,6-(Cl)₃—Ph |
| 296 | 8-Br | H | ethyl | ethyl | (CH₂)₃ | trans-CH═CH | 2,4,6-(Cl)₃—Ph |
| 297 | 5-CF₃ | H | ethyl | ethyl | (CH₂)₃ | trans-CH═CH | 2,4,6-(Cl)₃—Ph |
| 298 | 6-CF₃ | H | ethyl | ethyl | (CH₂)₃ | trans-CH═CH | 2,4,6-(Cl)₃—Ph |
| 299 | 7-CF₃ | H | ethyl | ethyl | (CH₂)₃ | trans-CH═CH | 2,4,6-(Cl)₃—Ph |
| 300 | 8-CF₃ | H | ethyl | ethyl | (CH₂)₃ | trans-CH═CH | 2,4,6-(Cl)₃—Ph |

TABLE 2

| No. | R₁ | R₂ | R₃ | R₄ | B | L (n = 1) | Ar |
|---|---|---|---|---|---|---|---|
| 301 | 7-Cl | H | CH₃ | CH₃ | (CH₂)₃ | trans- | 4-Br—Ph |
| | | | | | | CH═CH | |
| 302 | 8-NO₂ | H | CH₃ | CH₃ | (CH₂)₃ | trans- | 3,4-(Cl)₂—Ph |
| | | | | | | CH═CH | |
| 303 | H | H | CH₃ | CH₃ | (CH₂)₃ | trans- | 3,4-(Cl)₂—Ph |
| | | | | | | CH═CH | |
| 304 | 6-CH₃ | H | CH₃ | CH₃ | (CH₂)₃ | trans- | 3,4-(Cl)₂—Ph |
| | | | | | | CH═CH | |
| 305 | 7-Cl | H | ethyl | ethyl | CH(CH₃)(CH₂)₃ | trans- | 2-naphthyl |
| | | | | | | CH═CH | |
| 306 | 7-Cl | H | ethyl | ethyl | CH(CH₃)(CH₂)₃ | trans- | 3-Br—Ph |
| | | | | | | CH═CH | |
| 307 | 7-Cl | H | ethyl | ethyl | CH(CH₃)(CH₂)₃ | trans- | 4-CF₃—Ph |
| | | | | | | CH═CH | |
| 308 | 7-Cl | H | ethyl | ethyl | CH(CH₃)(CH₂)₃ | trans- | 4-NO₂—Ph |
| | | | | | | CH═CH | |
| 309 | 7-Cl | H | ethyl | ethyl | CH(CH₃)(CH₂)₃ | trans- | 4-CO₂CH₃—Ph |
| | | | | | | CH═CH | |
| 310 | 7-Cl | H | ethyl | ethyl | CH(CH₃)(CH₂)₃ | trans- | 4-Cl—Ph |
| | | | | | | CH═CH | |
| 311 | H | H | ethyl | ethyl | CH(CH₃)(CH₂)₃ | trans- | Ph |
| | | | | | | CH═CH | |
| 312 | 8-CF₃ | H | ethyl | ethyl | CH(CH₃)(CH₂)₃ | trans- | 3,4-(Cl)₂—Ph |
| | | | | | | CH═CH | |
| 313 | 8-CH₃ | H | ethyl | ethyl | CH(CH₃)(CH₂)₃ | trans- | 3,4-(Cl)₂—Ph |
| | | | | | | CH═CH | |
| 314 | 8-NO₂ | H | ethyl | ethyl | CH(CH₃)(CH₂)₃ | trans- | 3,4-(Cl)₂—Ph |
| | | | | | | CH═CH | |
| 315 | 7-CF₃ | H | ethyl | ethyl | CH(CH₃)(CH₂)₃ | trans- | 3,4-(Cl)₂—Ph |
| | | | | | | CH═CH | |
| 316 | 8-Br | H | ethyl | ethyl | CH(CH₃)(CH₂)₃ | trans- | 3,4-(Cl)₂—Ph |
| | | | | | | CH═CH | |
| 317 | 6-Cl | H | ethyl | ethyl | CH(CH₃)(CH₂)₃ | trans- | 3,4-(Cl)₂—Ph |
| | | | | | | CH═CH | |
| 318 | 6-Br | H | ethyl | ethyl | CH(CH₃)(CH₂)₃ | trans- | 3,4-(Cl)₂—Ph |
| | | | | | | CH═CH | |
| 319 | 8-F | H | ethyl | ethyl | CH(CH₃)(CH₂)₃ | trans- | 3,4-(Cl)₂—Ph |
| | | | | | | CH═CH | |
| 320 | 8-Cl | H | ethyl | ethyl | CH(CH₃)(CH₂)₃ | trans- | 3,4-(Cl)₂—Ph |
| | | | | | | CH═CH | |
| 321 | 6,7-(F)₂ | H | ethyl | ethyl | CH(CH₃)(CH₂)₃ | trans- | 3,4-(Cl)₂—Ph |
| | | | | | | CH═CH | |
| 322 | H | H | ethyl | ethyl | CH(CH₃)(CH₂)₃ | trans- | 3,4-(Cl)₂—Ph |
| | | | | | | CH═CH | |
| 323 | 6-SO₂CH₃ | H | ethyl | ethyl | CH(CH₃)(CH₂)₃ | trans- | 3,4-(Cl)₂—Ph |
| | | | | | | CH═CH | |
| 324 | 6-F | H | ethyl | ethyl | CH(CH₃)(CH₂)₃ | trans- | 3,4-(Cl)₂—Ph |
| | | | | | | CH═CH | |
| 325 | 7-Cl | H | ethyl | ethyl | CH(CH₃)(CH₂)₃ | trans- | 3,4-(Cl)₂—Ph |
| | | | | | | CH═CH | |
| 326 | 6-CH₃ | H | CH₃ | CH₃ | (CH₂)₂ | trans- | 3,4-(Cl)₂—Ph |
| | | | | | | CH═CH | |
| 327 | 6,7-(Cl)₂ | CH₃ | CH₃ | CH₃ | (CH₂)₃ | trans- | 3,4-(Cl)₂—Ph |
| | | | | | | CH═CH | |
| 328 | 8-CF₃ | H | CH₃ | CH₃ | (CH₂)₃ | trans- | Ph |
| | | | | | | CH═CH | |
| 329 | 8-CF₃ | H | CH₃ | CH₃ | (CH₂)₃ | trans- | (3-Cl-4-F)—Ph |
| | | | | | | CH═CH | |
| 330 | 8-CF₃ | H | CH₃ | CH₃ | (CH₂)₃ | trans- | 3,4-(F)₂—Ph |
| | | | | | | CH═CH | |
| 331 | 8-CF₃ | H | CH₃ | CH₃ | (CH₂)₃ | trans- | 2,4,5- |
| | | | | | | CH═CH | (CF₃)₃—Ph |
| 332 | 8-CF₃ | H | CH₃ | CH₃ | (CH₂)₃ | trans- | (3-CF₃-4- |
| | | | | | | CH═CH | F)—Ph |
| 333 | 8-CF₃ | H | CH₃ | CH₃ | (CH₂)₃ | trans- | 3-pyridyl |
| | | | | | | CH═CH | |
| 334 | 8-CF₃ | H | CH₃ | CH₃ | (CH₂)₃ | trans- | 3-CN—Ph |
| | | | | | | CH═CH | |
| 335 | 8-CF₃ | H | CH₃ | CH₃ | (CH₂)₃ | trans- | 2,5-(Cl)₂—Ph |
| | | | | | | CH═CH | |
| 335a | 7-OCH₃, | H | CH₃ | CH₃ | (CH₂)₃ | trans- | 3,4-(Cl)₂—Ph |
| | 8-CH₃ | | | | | CH═CH | |

TABLE 3

| | | | | | | L | | |
|---|---|---|---|---|---|---|---|---|
| No. | R₁ | R₂ | R₃ | R₄ | B | (n = 1)^{†} | Ar | MS* |
| 336 | 8-CH₃ | H | CH₃ | CH₃ | (CH₂)₃ | n = 0 | [3-CF₃-5- | 466 |
| | | | | | | | (pyridin-4-yl)]- | |
| | | | | | | | Ph | |
| 337 | 8-Cl | H | CH₃ | CH₃ | (CH₂)₃ | n = 0 | (3-Br-5-CF₃)—Ph | 487/489 |
| 338 | 8-Br | H | CH₃ | CH₃ | (CH₂)₃ | n = 0 | (3-Br-5-CF₃)—Ph | 533 |
| 339 | 8-CH₃ | H | CH₃ | CH₃ | (CH₂)₃ | n = 0 | 3,5-(CF₃)₂—Ph | 457 |
| 340 | 8-Cl | H | CH₃ | CH₃ | (CH₂)₃ | n = 0 | (3-CF₃-5-Ph)—Ph | 485 |
| 341 | 8-CH₃ | H | CH₃ | CH₃ | (CH₂)₃ | n = 0 | [3-CF₃-5-(2-F—Ph)]—Ph | 483 |
| 342 | 8-CH₃ | CH₃ | H | CH₃ | (CH₂)₃ | n = 0 | (3-Br-5-CF₃)—Ph | 457/469 |
| 343 | 8-CH₃ | H | CH₃ | CH₃ | (CH₂)₃ | n = 0 | (3-CF₃-5-Ph)—Ph | 465 |
| 344 | 8-CH₃ | H | CH₃ | CH₃ | (CH₂)₃ | n = 0 | 3-[(3-F-5- | 483 |
| | | | | | | | CF₃)—Ph]—Ph | |
| 345 | 8-CH₃ | H | CH₃ | CH₃ | (CH₂)₂ | n = 0 | (3-Br-5-CF₃)—Ph | 453/455 |
| 346 | 8-CH₃ | H | CH₃ | CH₃ | (CH₂)₃ | n = 0 | [3-CF₃-5-(5- | 472 |
| | | | | | | | thiazolyl)]-Ph | |
| 347 | 8-CH₃ | H | —(CH₂)₅— | | (CH₂)₃ | n = 0 | (3-Br-5-CF₃)—Ph | 507/509 |
| 348 | 8-CH₃ | H | —(CH₂)₂NH(CH₂)₂— | | (CH₂)₃ | n = 0 | (3-Br-5-CF₃)—Ph | 508/509 |
| 349 | 8-CH₃ | CH₃ | H | CH₃ | (CH₂)₃ | n = 0 | (3-CF₃-5-Ph)—Ph | 465 |
| 350 | 8-Cl | CH₃ | H | CH₃ | (CH₂)₃ | n = 0 | 3-[(3-F-5- | 503 |
| | | | | | | | CF₃)—Ph]—Ph | |
| 351 | 8-Cl | H | CH₃ | CH₃ | (CH₂)₃ | n = 0 | 3-(2-Cl—Ph)Ph | 451 |
| 352 | 8-CH₃ | H | ethyl | ethyl | (CH₂)₃ | n = 0 | (3-Br-5-CF₃)—Ph | 495/497 |
| 353 | 8-CH₃ | H | CH₃ | CH₃ | (CH₂)₃ | n = 0 | (3-Br-5-CF₃)—Ph | 467/469 |
| 354 | 8-CH₃ | H | —(CH₂)₂NMe(CH₂)₂— | | (CH₂)₃ | n = 0 | (3-Br-5-CF₃)—Ph | 522/524 |
| 355 | 8-CH₃ | H | CH₃ | CH₃ | (CH₂)₃ | n = 0 | 3-[(3-F-4- | 483 |
| | | | | | | | CF₃)—Ph]—Ph | |

*MS = experimental mass spectrometry data; [M + H]
^{†}n = 1, unless otherwise noted

Exemplary compounds of formula IIa:

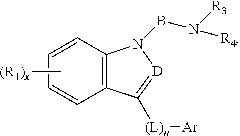

wherein D is CH, are depicted in Table 4.

TABLE 4

| No. | R₁ | R₃ | R₄ | B | L (n = 1)^{†} | Ar |
|---|---|---|---|---|---|---|
| 356 | 5-Br | CH₃ | CH₃ | (CH₂)₃ | trans- | 3,4-(Cl)₂—Ph |
| | | | | | CH═CH | |
| 357 | 6-F | —(CH₂)₂NMe(CH₂)₂— | | (CH₂)₂ | trans- | 3,4-(Cl)₂—Ph |
| | | | | | CH═CH | |
| 358 | 5-CH₃ | CH₃ | CH₃ | (CH₂)₃ | C(O)NH | 3,4-(Cl)₂—Ph |
| 359 | 5-Cl | CH₃ | CH₃ | (CH₂)₃ | n = 0 | 3,4-(Cl)₂—Ph |
| 360 | 5-Cl | H | H | C(O)CH₂ | n = 0 | 3,4-(Cl)₂—Ph |
| 361 | 5-Cl | H | H | C(O)CH(CH₃) | n = 0 | 3,4-(Cl)₂—Ph |
| 362 | 5-Cl | CH₃ | CH₃ | C(O)CH₂ | n = 0 | 3,4-(Cl)₂—Ph |
| 362a | 5-Cl | CH₃ | CH₃ | C(O)OCH₂CH₂ | n = 0 | 3,4-(Cl)₂—Ph |
| 362b | 5-Br | CH₃ | CH₃ | C(O)CH₂ | n = 0 | 3,4-(Cl)₂—Ph |
| 362c | 5-Cl | CH₃ | CH₃ | C(O)CH₂ | trans- | 3,4-(Cl)₂—Ph |
| | | | | | CH═CH | |
| 362d | 5-Br | H | H | C(O)CHCH₂CH₃ | trans- | 3,4-(Cl)₂—Ph |
| | | | | | CH═CH | |
| 362e | 5-Br | CH₃ | CH₃ | C(O)CH₂ | trans- | 3,4-(Cl)₂—Ph |
| | | | | | CH═CH | |
| 362f | 5-Cl | H | H | C(O)CHCH₂CH₃ | n = 0 | 3,4-(Cl)₂—Ph |
| 362g | 5-Cl | —(CH₂)₂NMe(CH₂)₂— | | C(O)CH₂ | n = 0 | 3,4-(Cl)₂—Ph |
| 362h | 5-CF₃ | CH₃ | CH₃ | C(O)CH₂ | trans- | 3,4-(Cl)₂—Ph |
| | | | | | CH═CH | |

^{†}n = 1, unless otherwise noted

Exemplary compounds of formula IIIa:

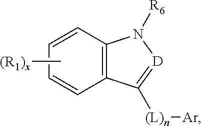

wherein D is CH, are listed in Table 5.

TABLE 5

| No. | R₁ | R₆ | L (n = 1)† | Ar | ¹H NMR* |
|---|---|---|---|---|---|
| 363 | 5-Br | H | trans-CH═CH | 3,4-(Cl)₂—Ph | 11.80 (bs, 1H, NH), |
| | | | | | 8.03-8.06 (s, 1H), 7.98- |
| | | | | | 8.01 (d, 1H), 7.88-7.92 |
| | | | | | (d, 1H), 7.64-7.73 (m, |
| | | | | | 3H). |
| 364 | 6-CF₃ | H | trans-CH═CH | 3,4-(Cl)₂—Ph | 11.81 (bs, 1H, NH), |
| | | | | | 8.24-8.27 (m, 1H), 7.90- |
| | | | | | 7.93 (m, 2H), 7.78 (s, |
| | | | | | 1H), 7.56-7.64 (m, 3H), |
| | | | | | 7.38-7.43 (m, 1H), 7.15 |
| | | | | | (d, J = 16.8 hz, 1H). |
| 365 | 5-Cl | H | trans-CH═CH | 3,4-(Cl)₂—Ph | 11.57 (bs, 1H, NH), |
| | | | | | 8.10-8.12 (m, 1H), 7.91- |
| | | | | | 7.94 (m, 1H), 7.74-7.78 |
| | | | | | (m, 1H), 7.52-7.62 (m, |
| | | | | | 3H), 7.42-7.50 (m, 1H), |
| | | | | | 7.14-7.18 (m, 1H), 7.07 |
| | | | | | (d, J = 16.6 hz, 1H). |
| 366 | 6-F | H | trans-CH═CH | 3,4-(Cl)₂—Ph | 11.40 (bs, 1H, NH), |
| | | | | | 8.02-8.08 (m, 1H), 7.87 |
| | | | | | (s, 1H), 7.65-7.69 (m, |
| | | | | | 1H), 7.50-7.60 (m, 3H), |
| | | | | | 7.18-7.24 (m, 1H), 7.09 |
| | | | | | (d, J = 16.4 hz, 1H), |
| | | | | | 6.94-7.02 (m, 1H). |
| 367 | 6-Br | H | trans-CH═CH | 3,4-(Cl)₂—Ph | 11.51 (bs, 1H, NH), 8.01 |
| | | | | | (d, 1H), 7.87 (s, 1H), |
| | | | | | 7.69 (s, 1H), 7.61-7.62 |
| | | | | | (m, 1H), 7.50-7.59 (m, |
| | | | | | 2H), 7.22-7.28 (m, 1H), |
| | | | | | 7.08 (d, J = 16.6 hz, 1H). |
| 368 | 5-F | H | trans-CH═CH | 3,4-(Cl)₂—Ph | 11.50 (bs, 1H, NH), |
| | | | | | 7.82-7.92 (m, 2H), 7.72- |
| | | | | | 7.78 (m, 1H), 7.50-7.62 |
| | | | | | (m, 3H), 7.40-7.46 (m, |
| | | | | | 1H), 6.98-7.10 (m, 2H). |
| 369 | 5-CF₃ | H | trans-CH═CH | 3,4-(Cl)₂—Ph | 11.8 (bs, 1H, NH), 8.38- |
| | | | | | 8.42 (m, 1H), 7.94-7.98 |
| | | | | | (m, 1H), 7.88-7.92 (s, |
| | | | | | 1H), 7.56-7.70 (m, 4H), |
| | | | | | 7,44-7.48 (m, 1H), 7.08- |
| | | | | | 7.16 (d, 1H). |
| 370 | 5-CH₃ | H | trans-CH═CH | 3,4-(Cl)₂—Ph | 11.2 (bs, 1H, NH), 7.82- |
| | | | | | 7.88 (m, 2H), 7.58-7.62 |
| | | | | | (m, 1H), 7.56-7.57 (m, |
| | | | | | 2H), 7.52 (d, 1H), 7.28- |
| | | | | | 7.32 (d, 1H), 6.98-7.08 |
| | | | | | (m, 2H). |
| 371 | 4-CF₃ | H | trans-CH═CH | 3,4-(Cl)₂—Ph | 12.0 (bs, 1H, NH), 8.01- |
| | | | | | 8.06 (m, 1H), 7.70-7.76 |
| | | | | | (m, 1H), 7.56-7.64 (m, |
| | | | | | 2H), 7.38-7.50 (m, 3H), |
| | | | | | 7.22-7.30 (m, 1H), 6.94- |
| | | | | | 7.02 (d, 1H). |
| 372 | 4-F | H | trans-CH═CH | 3,4-(Cl)₂—Ph | 11.7 (bs, 1H, NH), 7.78- |
| | | | | | 7.82 (m, 1H), 7.70-7.74 |
| | | | | | (m, 1H), 7.56-7.60 (m, |
| | | | | | 1H), 7.46-7.53 (m, 2H), |
| | | | | | 7.24-7.28 (m, 1H), 7.08- |
| | | | | | 7.15 (m, 1H), 7.00-7.07 |
| | | | | | (d, 1H), 6.82-6.90 (m, |
| | | | | | 1H). |
| 373 | 5-OCH₃ | H | trans-CH═CH | 3,4-(Cl)₂—Ph | 11.26 (bs, 1H, NH), |
| | | | | | 7.87-7.88 (m, 1H), 7.62- |
| | | | | | 7.66 (m, 1H), 7.46-7.60 |
| | | | | | (m, 4H), 7.30.7.34 (m, |
| | | | | | 1H), 6.96-7.04 (d, |
| | | | | | J = 16.4 hz, 1H), 6.80- |
| | | | | | 6.84 (m, 1H), 3.85 (s, |
| | | | | | 3H). |
| 374 | 5,6-(Cl)₂ | H | trans-CH═CH | 3,4-(Cl)₂—Ph | 11.61 (bs, 1H, NH), 8.13 |
| | | | | | (s, 1H), 7.92 (s, 1H), |
| | | | | | 7.78-7.82 (m, 1H), 7.67 |
| | | | | | (s, 1H), 7.52-7.62 (m, |
| | | | | | 3H), 7.10 (d, J = 16.4 hz, |
| | | | | | 1H) |
| 375 | 5-CN | H | trans-CH═CH | 3,4-(Cl)₂—Ph | 11.90 (bs, 1H, NH), |
| | | | | | 8.64-8.68 (m, 1H), 7.92- |
| | | | | | 7.96 (m, 1H), 7.86-7.88 |
| | | | | | (s, 1H), 7.56-7.64 (m, |
| | | | | | 4H), 7.50-7.53 (m, 1H), |
| | | | | | 7.20 (d, 1H). |
| 376 | 5-CO₂CH₃ | H | trans-CH═CH | 3,4-(Cl)₂—Ph | 11.80 (bs, 1H, NH), |
| | | | | | 8.64-8.68 (m, 1H), 7.92- |
| | | | | | 7.96 (m, 1H), 7.82-7.86 |
| | | | | | (m, 1H), 7.78-7.82 (m, |
| | | | | | 1H), 7.58-7.70 (m, 3H), |
| | | | | | 7.48-7.54 (m, 1H), 7.04- |
| | | | | | 7.10 (d, 1H). |
| 377 | H | H | trans-CH═CH | 3,4-(Cl)₂—Ph | 11.4 (bs, 1H, NH), 8.02- |
| | | | | | 8.07 (d, 1H), 7.86 (s, |
| | | | | | 1H), 7.64-7.68 (m, 1H), |
| | | | | | 7.50-7.60 (m, 3H), 7.40- |
| | | | | | 7.46 (d, 1H), 7.04-7.20 |
| | | | | | (m, 3H). |
| 378 | 6-F | H | trans-CH═CH | 3,4-(Br)₂—Ph | 11.42 (bs, 1H, NH), |
| | | | | | 8.03-8.08 (m, 1H), 7.98- |
| | | | | | 8.01 (m, 1H), 7.64-7.71 |
| | | | | | (m, 2H), 7.50-7.56 (m, |
| | | | | | 2H), 7.18-7.23 (m, 1H), |
| | | | | | 7.06 (d, J = 16.8 hz, 1H), |
| | | | | | 6.94-7.02 (m, 1H). |
| 379 | 5-NO₂ | H | trans-CH═CH | 3,4-(Cl)₂—Ph | 12.01 (bs, 1H, NH), 9.00 |
| | | | | | (s, 1H), 8.01-8.02 (m, |
| | | | | | 1H), 7.95-8.01 (s, 2H), |
| | | | | | 7.69-7.75 (d, J = 16.4 hz, |
| | | | | | 1H), 7.55-7.65 (m, 2H), |
| | | | | | 7.10-7.20 (d, J = 16.4 hz, |
| | | | | | 1H). |
| 380 | 5-NO₂ | H | cis-CH═CH | 3,4-(Cl)₂—Ph | 11.96 (bs, 1H, NH), |
| | | | | | 7.90-8.20 (m, 2H), 7.48- |
| | | | | | 7.62 (m, 4H), 7.28-7.32 |
| | | | | | (d, 1H), 6.90-6.98 (d, |
| | | | | | J = 12.1 hz, 1H), 6.54- |
| | | | | | 6.62 (d, J = 12.1 hz, 1H). |
| 381 | 7-Cl | H | trans-CH═CH | 3,4-(Cl)₂—Ph | 11.75 (bs, 1H, NH), |
| | | | | | 8.02-8.06 (d, 1H), 7.88 |
| | | | | | (s, 1H), 7.76 (s, 1H), |
| | | | | | 7.52-7.60 (m, 3H), 7.24- |
| | | | | | 7.28 (m, 1H), 7.10-7.17 |
| | | | | | (m, 2H). |
| 382 | 6-CN | H | trans-CH═CH | 3,4-(Cl)₂—Ph | 11.99 (bs, 1H, NH), 8.22 |
| | | | | | (d, J = 8.2 hz, 1H), 7.98 |
| | | | | | (s, 1H), 7.89-7.95 (m, |
| | | | | | 2H), 7.54-7.63 (m, 3H), |
| | | | | | 7.44-7.48 (m, 1H), 7.15 |
| | | | | | (d, J = 16.7 hz, 1H). |
| 383 | 6-F | H | trans-CH═CH | 2,3-(Cl)₂—Ph | 11.50 (bs, 1H, NH), |
| | | | | | 7.90-7.95 (m, 1H), 7.82- |
| | | | | | 7.88 (m, 1H), 7.74-7.78 |
| | | | | | (m, 1H), 7.44-7.56 (m, |
| | | | | | 2H), 7.30-7.40 (m, 2H), |
| | | | | | 7.22-7.28 (m, 1H), 7.00- |
| | | | | | 7.08 (m, 1H). |
| 384 | 6-F | H | trans-CH═CH | 2,5-(Cl)₂—Ph | 11.50 (bs, 1H, NH), |
| | | | | | 7.90-7.98 (m, 2H), 7.74- |
| | | | | | 7.78 (m, 1H), 7.58-7.66 |
| | | | | | (d, 1H), 7.46-7.50 (m, |
| | | | | | 1H), 7.20-7.30 (m, 3H), |
| | | | | | 7.00-7.08 (m, 1H). |
| 385 | 6-F | H | trans-CH═CH | (4-F-3-CF₃)—Ph | 11.40 (bs, 1H, NH), |
| | | | | | 8.07-8.11 (m, 1H), 7.90- |
| | | | | | 7.99 (m, 2H), 7.67 (s, |
| | | | | | 1H), 7.45-7.55 (m, 2H), |
| | | | | | 7.17-7.24 (m, 2H), 6.96- |
| | | | | | 7.02 (m, 1H). |
| 386 | 6-F | H | trans-CH═CH | 3,5-(CF₃)₂—Ph | 11.51 (bs, 1H, NH), |
| | | | | | 8.26-8.31 (m, 2H), 8.14- |
| | | | | | 8.20 (m, 1H), 7.76-7.84 |
| | | | | | (m, 2H), 7.70-7.74 (m, |
| | | | | | 1H), 7.33 (d, J = 16.4 hz, |
| | | | | | 1H), 7.22-7.27 (m, 1H), |
| | | | | | 6.98-7.05 (m, 1H). |
| 387 | 6-F | H | trans-CH═CH | 4-CF₃—Ph | 11.69 (bs, 1H, NH), |
| | | | | | 8.04-8.09 (m, 1H), 7.64- |
| | | | | | 7.82 (m, 5H), 7.80 (d, |
| | | | | | J = 16.4 hz, 1H), 7.15- |
| | | | | | 7.26 (m, 2H), 6.96-7.04 |
| | | | | | (m, 1H). |
| 388 | 6-F | H | trans-CH═CH | 3,5-(Cl)₂—Ph | 11.47 (bs, 1H, NH), |
| | | | | | 8.04-8.10 (m, 1H), 7.64- |
| | | | | | 7.70 (m, 3H), 7.60 (d, |
| | | | | | J = 16.4 hz, 1H), 7.32- |
| | | | | | 7.38 (m, 1H), 7.19-7.24 |
| | | | | | (m, 1H), 7.08 (d, J = 16.4 |
| | | | | | hz, 1H), 6.94-7.02 (m, |
| | | | | | 1H). |
| 389 | 6-F | H | trans-CH═CH | 3,5-(Br)₂—Ph | 11.42 (bs, 1H, NH), |
| | | | | | 8.04-8.10 (m, 1H), 7.82- |
| | | | | | 7.85 (m, 2H), 7.64-7.68 |
| | | | | | (m, 1H), 7.56-7.62 (m, |
| | | | | | 2H), 7.19-7.24 (m, 1H), |
| | | | | | 7.03-7.10 (d, 1H), 6.95- |
| | | | | | 7.02 (m, 1H). |
| 390 | 6-F | H | trans-CH═CH | 2,4-(Cl)₂—Ph | 11.50 (bs, 1H, NH), |
| | | | | | 7.86-7.94 (m, 2H), 7.72- |
| | | | | | 7.77 (m, 1H), 7.58-7.62 |
| | | | | | (m, 1H), 7.52 (d, J = 16.4 |
| | | | | | hz, 1H), 7.38-7.44 (m, |
| | | | | | 1H), 7.20-7.30 (m, 2H), |
| | | | | | 7.00-7.06 (m, 1H). |
| 391 | 6-F | H | trans-CH═CH | 2,3,5-(Cl)₃—Ph | |
| 392 | 6-F | H | trans-CH═CH | (3-CF₃-4-Cl)— | 11.42 (bs, 1H, NH), |
| | | | | Ph | 8.08-8.14 (m, 1H), 8.02- |
| | | | | | 8.06 (m, 1H), 7.88-7.94 |
| | | | | | (m, 1H), 7.55-8.06 (m, |
| | | | | | 1H), 7.88-7.94 (m, 1H), |
| | | | | | 7.55-7.72 (m, 3H), 7.16- |
| | | | | | 7.24 (m, 2H), 6.95-7.04 |
| | | | | | (m, 1H). |
| 393 | 6-F | H | trans-CH═CH | (3-Cl-4-CF₃)— | 11.51 (bs, 1H, NH), |
| | | | | Ph | 8.06-8.14 (m, 1H), 7.93- |
| | | | | | 7.97 (m, 1H), 7.65-7.81 |
| | | | | | (m, 4H), 7.21-7.26 (m, |
| | | | | | 1H), 7.16 (d, J = 16.8 hz, |
| | | | | | 1H), 6.96-7.04 (m, 1H). |
| 394 | 6-F | H | trans-CH═CH | 4-OCF₃—Ph | 11.43 (bs, 1H, NH), |
| | | | | | 8.00-8.08 (m, 1H), 7.64- |
| | | | | | 7.74 (m, 3H), 7.40-7.49 |
| | | | | | (m, 1H), 7.29-7.38 (m, |
| | | | | | 2H), 7.10, 7.26 (m, 2H), |
| | | | | | 6.94-7.03 (m, 1H). |
| 395 | 6-F | H | trans-CH═CH | 3-OCF₃—Ph | 11.45 (bs, 1H, NH), |
| | | | | | 8.02-8.10 (m, 1H), 7.66- |
| | | | | | 7.70 (m, 1H), 7.44-7.64 |
| | | | | | (m, 4H), 7.19-7.24 (m, |
| | | | | | 1H), 7.12-7.18 (m, 2H), |
| | | | | | 6.95-7.02 (m, 1H). |
| 396 | 6-F | H | trans-CH═CH | 3-CF₃—Ph | 11.41 (bs, 1H, NH), |
| | | | | | 8.06-8.12 (m, 1H), 7.86- |
| | | | | | 7.96 (m, 2H), 7.66-7.70 |
| | | | | | (m, 1H), 7.50-7.62 (m, |
| | | | | | 3H), 7.19-7.23 (m, 2H), |
| | | | | | 6.94-7.02 (m, 1H). |
| 397 | 6-F | H | trans-CH═CH | 3,4,5-(Cl)₃—Ph | 11.50 (bs, 1H, NH), |
| | | | | | 8.04-8.10 (m, 1H), 7.86- |
| | | | | | 7.90 (m, 2H), 7.60-7.70 |
| | | | | | (m, 3H), 7.18-7.25 (m, |
| | | | | | 1H), 7.03-7.10 (d, 1H), |
| | | | | | 6.96-7.02 (m, 1H). |
| 398 | 6-F | H | trans-CH═CH | 3,4-(F)₂—Ph | 11.0 (bs, 1H, NH), 7.98- |
| | | | | | 8.06 (m, 1H), 7.62-7.72 |
| | | | | | (m, 2H), 7.36-7.46 (m, |
| | | | | | 3H), 7.18-7.23 (m, 1H), |
| | | | | | 7.07 (d, J = 16.8 hz, 1H), |
| | | | | | 6.94-7.01 (m, 1H). |
| 399 | 6-F | H | trans-CH═CH | 3-F—Ph | 11.40 (bs, 1H, NH), |
| | | | | | 7.98-8.06 (m, 1H), 7.64- |
| | | | | | 7.68 (s, 1H), 7.30-7.52 |
| | | | | | (m, 4H), 7.16-7.24 (m, |
| | | | | | 1H), 7.04-7.14 (d, 1H), |
| | | | | | 6.94-7.04 (m, 2H). |
| 400 | 6-F | H | trans-CH═CH | 4-CH₂OH—Ph | 11.35 (bs, 1H, NH), |
| | | | | | 7.96-8.02 (m, 1H), 7.63 |
| | | | | | (s, 1H), 7.50-7.56 (m, |
| | | | | | 2H), 7.37 (d, J = 16.4 hz, |
| | | | | | 1H), 7.28-7.32 (m, 2H), |
| | | | | | 7.16-7.22 (m, 1H), 7.08 |
| | | | | | (d, J = 16.8 hz, 1H), 6.92- |
| | | | | | 7.00 (m, 1H), 5.14 (t, |
| | | | | | 1H, OH), 4.48-4.52 (d, |
| | | | | | 2H). |
| 401 | 6-F | H | trans-CH═CH | 3,4-(CH₃)₂—Ph | 11.30 (bs, 1H, NH), |
| | | | | | 7.94-8.01 (m, 1H), 7.58- |
| | | | | | 7.62 (m, 1H), 7.24-7.38 |
| | | | | | (m, 3H), 7.04-7.14 (d, |
| | | | | | 1H), 6.94-7.04 (m, 2H). |
| 402 | 6-F | H | trans-CH═CH | 3-Cl—Ph | 11.40 (bs, 1H, NH), |
| | | | | | 8.02-8.08 (m, 1H), 7.62- |
| | | | | | 7.70 (m, 2H), 7.46-7.56 |
| | | | | | (m, 2H), 7.30-7.40 (m, |
| | | | | | 1H), 7.18-7.26 (m, 2H), |
| | | | | | 7.09 (d, J = 16.4 hz, 1H), |
| | | | | | 6.92-7.02 (m, 1H). |
| 403 | 6-F | H | trans-CH═CH | 4-Cl—Ph | 11.42 (bs, 1H, NH), |
| | | | | | 8.79.8.06 (m, 1H), 7.60- |
| | | | | | 7.80 (m, 1H), 7.58-7.63 |
| | | | | | (m, 2H), 7.36-7.48 (m, |
| | | | | | 3H), 7.20-7.26 (m, 1H), |
| | | | | | 7.10 (d, J = 16.4 hz, 1H), |
| | | | | | 6.95-7.02 (m, 1H). |
| 404 | 6-F | H | trans-CH═CH | 3-Br—Ph | 11.42 (bs, 1H, NH), |
| | | | | | 8.02-8.08 (m, 1H), 7.80- |
| | | | | | 7.84 (m, 1H), 7.64-7.68 |
| | | | | | (m, 1H), 7.55-7.60 (m, |
| | | | | | 1H), 7.46-7.52 (d, 1H), |
| | | | | | 7.33-7.38 (m, 1H), 7.26- |
| | | | | | 7.33 (m, 1H), 7.18-7.24 |
| | | | | | (m,1H), 7.04-7.12 (d, |
| | | | | | 1H), 6.94-7.02 (m, 1H). |
| 405 | 6-F | H | trans-CH═CH | 4-Br—Ph | 11.40 (bs, 1H, NH), |
| | | | | | 8.00-8.04 (m, 1H), 7.64- |
| | | | | | 7.68 (m, 1H). 7.49-7.58 |
| | | | | | (m, 4H), 7.50 (d, J = 16.4 |
| | | | | | hz, 1H), 7.18-7.25 (m, |
| | | | | | 1H), 7.07 (d, J = 16.4 hz, |
| | | | | | 1H), 6.94-7.01 (m, 1H). |
| 406 | 6-F | H | trans-CH═CH | 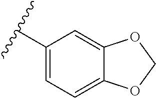 [SMILES: *c1ccc2c(c1)OCO2] | 11.31 (bs, 1H, NH), 7.58-8.00 (m, 1H) 7.55- 7.60 (m, 1H), 7.14-7.30 (m, 3H), 6.84-7.06 (m, 4H), 6.02 (s, 2H). |
| 407 | 6-F | H | trans-CH═CH | 3-NO₂—Ph | 11.42 (bs, 1H, NH), |
| | | | | | 8.40-8.46 (m, 1H), 8.90- |
| | | | | | 8.96 (m, 1H), 7.98-8.08 |
| | | | | | (m, 2H), 7.70-7.74 (m, |
| | | | | | 1H), 7.60-7.68 (m, 2H), |
| | | | | | 7.20-7.30 (m, 2H), 6.96- |
| | | | | | 7.24 (m, 1H). |
| 408 | 6-F | H | trans-CH═CH | 3-pyridyl | 11.40 (bs, 1H, NH), |
| | | | | | 8.74-8.78 (m, 1H), 8.34- |
| | | | | | 8.40 (m, 1H), 7.96-8.06 |
| | | | | | (m, 2H), 7.68 (s, 1H), |
| | | | | | 7.50-7.58 (d, 1H), 7.32- |
| | | | | | 7.38 (m, 1H), 7.18-7.24 |
| | | | | | (m, 1H), 7.06-7.14 (d, |
| | | | | | 1H), 6.98-7.20 (m, 1H). |
| 409 | 6-F | H | trans-CH═CH | 3-CN—Ph | 11.45 (bs, 1H, NH), |
| | | | | | 8.02-8.10 (m, 2H), 7.86- |
| | | | | | 7.92 (m, 1H), 7.66-7.70 |
| | | | | | (m, 1H), 7.50-7.64 (m, |
| | | | | | 3H), 7.18-7.24 (m, 1H), |
| | | | | | 7.13 (d, J = 16.7 hz, 1H), |
| | | | | | 6.95-7.03 (m, 1H). |
| 410 | 6-F | H | trans-CH═CH | 3-(NH₂CO)—Ph | |
| 411 | 5-Br | H | (CH₂)₂ | 3,4-(Cl)₂—Ph | 10.98 (bs, 1H, NH), |
| | | | | | 7.68-7.72 (m, 1H), 7.53- |
| | | | | | 7.56 (m, 1H), 7.48-7.52 |
| | | | | | (d, 1H), 7.28-7.32 (d, |
| | | | | | 1H), 7.22-7.26 (m, 1H), |
| | | | | | 7.12-7.18 (m, 2H), 2.90- |
| | | | | | 3.01 (m, 4H). |
| 412 | H | H | (CH₂)₂ | 3,4-(Cl)₂—Ph | 10.76 (bs, 1H, NH), |
| | | | | | 7.40-7.60 (m, 3H), 7.20- |
| | | | | | 7.38 (m, 2H), 7.00-7.10 |
| | | | | | (m, 2H), 6.90-7.00 (m, |
| | | | | | 1H), 2.90-3.36 (m, 4H). |
| 413 | 6-F | CH3 | trans-CH═CH | 3,4-(Cl)₂—Ph | 8.04-8.10 (m, 1H), 7.85- |
| | | | | | 7.89 (s, 1H), 7.62-7.65 |
| | | | | | (s, 1H), 7.55-7,59 (m, |
| | | | | | 2H), 7.50 (d, J = 16.8 hz, |
| | | | | | 1H), 7.34-7.39 (m, 1H), |
| | | | | | 7.06 (d, J = 16.8 hz, 1H), |
| | | | | | 6.98-7.04 (m, 1H), 3.56 |
| | | | | | (s, 3H). |
| 414 | 6-F | 4-F-benzyl | trans-CH═CH | 3,4-(Cl)₂—Ph | 8.04-8.12 (m, 1H), 7.89 |
| | | | | | (s, 1H), 7.81 (s, 1H), |
| | | | | | 7.42-7.60 (m, 4H), 7.30- |
| | | | | | 7.38 (m, 2H), 7.14-7.20 |
| | | | | | (m, 2H), 7.06-7.13 (d, |
| | | | | | J = 16.8 hz, 1H), 6.98- |
| | | | | | 7.04 (m, 1H), 5.41 (s, |
| | | | | | 2H). |
| 415 | 6-F | (CH2)2Cl | trans-CH═CH | 3,4-(Cl)₂—Ph | 8.04-8.10 (m, 1H), 7.89 |
| | | | | | (s, 1H), 7.75 (s, 1H), |
| | | | | | 7.45-7.60 (m, 4H), 7.00- |
| | | | | | 7.12 (m, 2H), 4.53-4.58 |
| | | | | | (t, 2H), 3.90-4.04 (t, |
| | | | | | 2H). |
| 416 | 6-F | CH2CO2H | trans-CH═CH | 3,4-(Cl)₂—Ph | 8.00-8.10 (m, 1H), 7.85- |
| | | | | | 7.90 (s, 1H), 7.46-7.65 |
| | | | | | (m, 4H), 7.30-7.40 (m, |
| | | | | | 1H), 6.95-7.10 (m, 2H), |
| | | | | | 4.96 (s, 2H). |
| 417 | 6-F | (CH2)2CO2H | trans-CH═CH | 3,4-(Cl)₂—Ph | 12.40 (bs, 1H, —OH), |
| | | | | | 8.02-8.08 (m, 1H), 7.8 |
| | | | | | (s, 1H), 7.68 (s, 1H), |
| | | | | | 7.56-7.60 (m, 2H), 7.43- |
| | | | | | 7.53 (m, 2H), 6.98-7.10 |
| | | | | | (m, 2H), 4.39 (t, 2H), |
| | | | | | 2.78 (t, 2H). |
| 418 | 5-Br | H | CH₂NH | 3,4-(Cl)₂—Ph | 11.13 (bs, 1H, NH), |
| | | | | | 7.81-7.82 (m, 1H), 7.40- |
| | | | | | 7.42 (m, 1H), 7.30-7.36 |
| | | | | | (m, 1H), 7.16-7.24 (m, |
| | | | | | 2H), 6.82-6.85 (m, 1H), |
| | | | | | 6.62-6.68 (m, 1H), 6.44- |
| | | | | | 6.50 (m, 1H), 4.30-4.40 |
| | | | | | (m, 2H). |
| 419 | 5-Br | H | C(O)NHCH₂ | 3,4-(Cl)₂—Ph | 11.8 (bs, 1H, NH), 8.6 |
| | | | | | (t, 1H, NH), 8.28-8.32 |
| | | | | | (m, 1H), 8.10-8.14 (m, |
| | | | | | 1H), 7.50-7.72 (m, 2H), |
| | | | | | 7.40-7.46 (m, 1H), 7.26- |
| | | | | | 7.36 (m, 3H), 4.40-4.50 |
| | | | | | (m, 2H). |
| 420 | 5-Br | H | C(O)NH | 3,4-(Cl)₂—Ph | 12.00 (bs, 1H, NH), |
| | | | | | 10.04 (bs, 1H, NH), |
| | | | | | 8.32-8.38 (m, 2H), 8.16- |
| | | | | | 8.20 (m, 1H), 7.69-7.75 |
| | | | | | (m, 1H), 7.57-7.62 (d, |
| | | | | | 1H), 7.46-7.50 (d, 1H), |
| | | | | | 7.31-7.36 (m, 1H). |
| 421 | 5-CH₃ | H | C(O)NH | 3,4-(Cl)₂—Ph | |
| 422 | 5-Br | H | C(O)C(O)NH | 3,4-(Cl)₂—Ph | |
| 423 | 5-Br | H | C(O)NH | 3-(4-chloro- | |
| | | | | pyridazine) | |
| 424 | 6-Br | H | C(O)NH | 3,4-(Cl)₂—Ph | |
| 425 | 5-Cl | H | C(O)NH | 3,4-(Cl)₂—Ph | |
| 426 | 5-Cl | H | C(O) | 3,4-(Cl)₂—Ph | |
| 427 | 5-Cl | H | C(O)NH | 2-(3,4-(Cl)₂- | |
| | | | | pyridine) | |
| 428 | 5-Br | H | C(O)NH | 3-CF₃—Ph | |
| 429 | 5-Cl | H | n = 0 | 3,4-(Cl)₂—Ph | 11.75 (bs, 1H, NH), |
| | | | | | 7.92-7.98 (m, 2H), 7.87- |
| | | | | | 7.91 (m, 1H), 7.82-7.86 |
| | | | | | (m, 1H), 7.64-7.72 (m, |
| | | | | | 2H), 7.46-7.51 (m, 1H), |
| | | | | | 7.16-7.21 (m, 1H). |
| 430 | 5-Cl | H | n = 0 | 3-CF₃—Ph | 11.75 (bs, 1H, NH), |
| | | | | | 7.96-8.04 (m, 2H), 7.90- |
| | | | | | 7.93 (m, 1H), 7.82-7.85 |
| | | | | | (m, 1H), 7.64-7.71 (m, |
| | | | | | 1H), 7.56-7.62 (m, 1H), |
| | | | | | 7.47-7.53 (m, 1H), 7.17- |
| | | | | | 7.23 (m, 1H). |
| 431 | 5,6-(Cl)₂ | H | n = 0 | 3-CF₃—Ph | 11.80 (bs, 1H, NH), |
| | | | | | 7.98-8.05 (m, 2H), 7.90- |
| | | | | | 7.95 (s, 1H), 7.65-7.74 |
| | | | | | (m, 2H), 7.58-7.63 (m, |
| | | | | | 1H). |
| 432 | 5,6-(Cl)₂ | H | n = 0 | 3,4-(Cl)₂—Ph | 11.80 (bs, 1H, NH), |
| | | | | | 8.03-8.06 (s, 1H), 7.98- |
| | | | | | 8.01 (d, 1H), 7.88-7.92 |
| | | | | | (d, 1H), 7.64-7.73 (m, |
| | | | | | 3H). |
| 433 | 6-Cl | H | n = 0 | 3,4-(Cl)₂—Ph | 11.7 (bs, 1H, NH), 7.84- |
| | | | | | 7.94 (m, 3H), 7.64-7.73 |
| | | | | | (m, 2H), 7.50-7.53 (m, |
| | | | | | 1H), 7.12-7.17 (m, 1H). |
| 434 | 5,6-(Cl)₂ | H | n = 0 | (3-Cl-5-CF₃)— | 11.90 (bs, 1H, NH), |
| | | | | Ph | 8.11-8.13 (s, 1H), 8.02- |
| | | | | | 8.08 (m, 2H), 7.91-7.94 |
| | | | | | (s, 1H), 7.72-7.75 (s, |
| | | | | | 1H), 7.69-7.71 (s, 1H). |
| 435 | 5,6-(Cl)₂ | H | n = 0 | 3,5-(CF₃)₂—Ph | 12.00 (bs, 1H, NH), |
| | | | | | 8.24-8.28 (s, 2H), 8.18- |
| | | | | | 8.21 (s, 1H), 8.02-8.04 |
| | | | | | (s, 1H), 7.92-7.95 (s, |
| | | | | | 1H), 7.74-7.76 (s, 1H). |
| 436 | 5,6-(Cl)₂ | H | n = 0 | (3-CF₃-4-Cl)— | 11.90 (bs, 1H, NH), |
| | | | | Ph | 8.06-8.09 (m, 1H), 7.99- |
| | | | | | 8.05 (m, 3H), 7.72-7.78 |
| | | | | | (m, 2H). |
| 437 | 5-[3,4- | H | n = 0 | 3,4-(Cl)₂—Ph | 11.65 (bs, 1H, NH), 8.08 |
| | (Cl)₂—Ph]- | | | | (s, 1H), 7.90-7.99 (m, |
| | | | | | 3H), 7.79-7.85 (m, 1H), |
| | | | | | 7.64-7.74 (m, 3H), 7.56 |
| | | | | | (d, 1H), 7.48-7.52 (m, |
| | | | | | 1H). |
| 438 | 5-(3-CF₃— | H | n = 0 | 3,4-(Cl)₂—Ph | 11.7 (bs, 1H, NH), 8.08- |
| | Ph)- | | | | 8.12 (m, 1H), 8.02-8.06 |
| | | | | | (m, 1H), 7.92-8.00 (m, |
| | | | | | 3H), 7.79-7.84 (m, 1H), |
| | | | | | 7.66-7.73 (m, 3H), 7.50- |
| | | | | | 7.62 (m, 2H). |
| 439 | 5-Br | H | C(O)CH₂ | 3,4-(Cl)₂—Ph | 12.25 (bs, 1H, NH), |
| | | | | | 8.58-8.61 (m, 1H), 8.28- |
| | | | | | 8.30 (m, 1H), 7.61-7.64 |
| | | | | | (m, 1H), 7.55-7.59 (d, |
| | | | | | 1H), 7.45-7.49 (d, 1H), |
| | | | | | 7.30-7.38 (m, 3H), 4.24 |
| | | | | | (s, 2H). |
| 440 | 5,6-(Cl)₂ | H | C(CH₃)₂ | 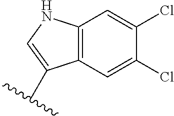 [SMILES: *c1c[nH]c2cc(Cl)c(Cl)cc12] | 11.19 (bs, 2H, NH), 7.56 (s, 2H), 7.52 (m, 1H), 7.14 (s, 2H), 1.77 (s, 6H). |
| 440a | 5-Cl | 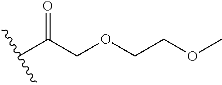 [SMILES: *C(=O)COCCOC] | n = 0 | 3,4-(Cl)₂—Ph | |

*400 MHz, DMSO-d₆ solvent
†n = 1, unless otherwise noted

An exemplary compound of formula IIIa:

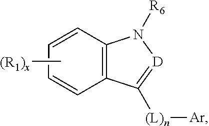

wherein D is N and n=0, is listed in Table 6.

TABLE 6

| No. | R₁ | R₆ | Ar | MS* |
|---|---|---|---|---|
| 441 | 6-CF₃ | H | 3,4-(Cl)₂—Ph | 331 |

*MS = experimental mass spectrometry data; [M + H]

An exemplary compound of formula IIIa:

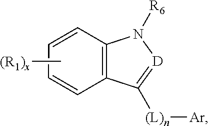

wherein D is C(CH₃), is listed in Table 7.

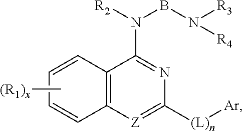

Table 8 depicts compounds of Formula Ia:

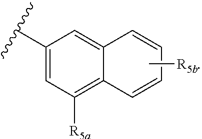

wherein Z is N, n is 0, and Ar is

TABLE 7

| No. | R₁ | R₆ | L (n = 1)^{†} | Ar |
|---|---|---|---|---|
| 442 | 5-Cl | H | trans-CH═CH | 3,4-(Cl)₂—Ph |

^{†}n = 1, unless otherwise noted

TABLE 8

| No. | R₁ | R₂ | R₃ | R₄ | B | R₅ₐ | R_{5b} | MS* |
|---|---|---|---|---|---|---|---|---|
| 443 | 8-CH₃ | H | ethyl | ethyl | CH(CH₃)(CH₂)₃ | H | 6,7-(Cl)₂ | 495 |
| 444 | 8-CH₃ | H | CH₃ | CH₃ | (CH₂)₃ | H | 6,7-(Cl)₂ | 439 |
| 445 | 8-CH₃ | H | ethyl | ethyl | CH(CH₃)(CH₂)₃ | Br | H | 505/507 |
| 446 | 8-CH₃ | H | CH₃ | CH₃ | (CH₂)₃ | Br | H | 449/451 |
| 447 | 8-CH₃ | H | ethyl | ethyl | CH(CH₃)(CH₂)₃ | benzyl | H | 517 |
| 448 | 8-CH₃ | H | CH₃ | CH₃ | (CH₂)₃ | benzyl | H | 461 |
| 449 | 8-(3-pyridyl) | H | ethyl | ethyl | CH(CH₃)(CH₂)₃ | H | H | 490 |
| 450 | 8-(3-pyridyl) | H | CH₃ | CH₃ | (CH₂)₃ | H | H | 434 |
| 451 | 8-CH₃ | H | ethyl | ethyl | CH(CH₃)(CH₂)₃ | benzyl | 6,7-(Cl)₂ | 585 |
| 452 | 8-CH₃ | H | CH₃ | CH₃ | (CH₂)₃ | benzyl | 6,7-(Cl)₂ | 529 |
| 453 | 8-CH₃ | H | ethyl | ethyl | CH(CH₃)(CH₂)₃ | H | C6-C7 C₄H₄ | 577 |
| 454 | 8-CH₃ | H | CH₃ | CH₃ | (CH₂)₃ | H | C6-C7 C₄H₄ | 421 |

*MS = experimental mass spectrometry data; [M + H]

Table 9 depicts compounds of Formula Ia:

TABLE 9

| No. | R₁ | R₂ | R₃ | R₄ | B | Ar | MS* |
|---|---|---|---|---|---|---|---|
| 455 | 8-CH₃ | H | Me | Me | (CH₂)₃ | 3-Br—Ph | 399/401 |
| 456 | 8-CH₃ | H | Me | Me | (CH₂)₃ | (2-Br-5-CF₃)—Ph | 467/469 |
| 457 | 8-CH₃ | H | Me | Me | (CH₂)₃ | (4-Br-5-CF₃)—Ph | 467/469 |
| 458 | 8-CH₃ | H | Me | Me | (CH₂)₃ | 3,4-(Cl)₂—Ph | 389 |
| 459 | 8-CH₃ | H | —(CH₂)₂O(CH₂)₂— | | (CH₂)₃ | (3-Br-5-CF₃)—Ph | 509/511 |
| 460 | 8-CH₃ | H | —(CH₂)₂NMe(CH₂)₂— | | (CH₂)₃ | (3-Br-5-CF₃)—Ph | 522/524 |
| 461 | 8-CH₃ | H | —(CH₂)₅— | | (CH₂)₃ | (3-Br-5-CF₃)—Ph | 507/509 |
| 462 | 8-CH₃ | H | —(CH₂)₂NH(CH₂)₂— | | (CH₂)₃ | (3-Br-5-CF₃)—Ph | 508/50 |
| 463 | 8-CH₃ | H | Me | Me | (CH₂)₃ | 4-[(3-F-5-CF₃)—Ph]—Ph | 483 |
| 464 | 8-CH₃ | H | Me | Me | (CH₂)₃ | 4-[(2-F-5-CF₃)—Ph]—Ph | 483 |

*MS = experimental mass spectrometry data; [M + H]

wherein Z is N, n is 0.

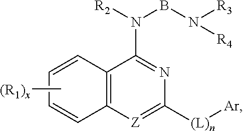

Table 10 depicts compounds of Formula Ia:

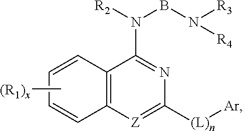

wherein Z is N, n is 0, and Ar is

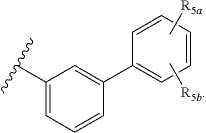

TABLE 10

| No. | R₁ | R₂ | R₃ | R₄ | B | R₅ₐ | R_{5b} | MS* |
|---|---|---|---|---|---|---|---|---|
| 465 | 8-CH₃ | H | ethyl | ethyl | CH(CH₃)(CH₂)₃ | 3-CF₃ | — | 521 |
| 466 | 8-CH₃ | H | CH₃ | CH₃ | (CH₂)₃ | 3-CF₃ | — | 465 |
| 467 | 8-CH₃ | H | ethyl | ethyl | CH(CH₃)(CH₂)₃ | 3-Cl | 4-Cl | 521 |
| 468 | 8-CH₃ | H | CH₃ | CH₃ | (CH₂)₃ | 3-Cl | 4-Cl | 465 |
| 469 | 8-CH₃ | H | ethyl | ethyl | CH(CH₃)(CH₂)₃ | 3-Cl | 5-Cl | 521 |
| 470 | 8-CH₃ | H | CH₃ | CH₃ | (CH₂)₃ | 3-Cl | 5-Cl | 465 |
| 471 | 8-CH₃ | H | ethyl | ethyl | CH(CH₃)(CH₂)₃ | 2-F | 4-CF₃ | 539 |
| 472 | 8-CH₃ | H | CH₃ | CH₃ | (CH₂)₃ | 2-F | 4-CF₃ | 483 |
| 473 | 8-CH₃ | H | ethyl | ethyl | CH(CH₃)(CH₂)₃ | 2-F | 5-CF₃ | 539 |
| 474 | 8-CH₃ | H | CH₃ | CH₃ | (CH₂)₃ | 2-F | 5-CF₃ | 483 |
| 475 | 8-CH₃ | H | ethyl | ethyl | CH(CH₃)(CH₂)₃ | 3-F | 5-CF₃ | 539 |
| 476 | 8-CH₃ | H | ethyl | ethyl | CH(CH₃)(CH₂)₃ | 3-F | 4-CF₃ | 539 |
| 477 | 8-CH₃ | CH₃ | CH₃ | CH₃ | (CH₂)₃ | 3-CF₃ | — | 479 |
| 478 | 8-CH₃ | H | H | CH₃ | (CH₂)₃ | 3-CF₃ | — | 451 |
| 479 | 7-CF₃ | H | ethyl | ethyl | CH(CH₃)(CH₂)₃ | — | — | 507 |
| 480 | 7-CF₃ | H | CH₃ | CH₃ | (CH₂)₃ | — | — | 451 |
| 481 | 7-CF₃ | H | ethyl | ethyl | CH(CH₃)(CH₂)₃ | 2-F | 5-CF₃ | 593 |
| 482 | 7-CF₃ | H | CH₃ | CH₃ | (CH₂)₃ | 2-F | 5-CF₃ | 537 |
| 483 | 8-CH₃ | H | ethyl | ethyl | CH(CH₃)(CH₂)₃ | 2-Cl | 3-Cl | 521 |
| 484 | 8-CH₃ | H | CH₃ | CH₃ | (CH₂)₃ | 2-Cl | 3-Cl | 465 |
| 485 | 8-Cl | H | ethyl | ethyl | CH(CH₃)(CH₂)₃ | — | — | 473 |
| 486 | 8-Cl | H | CH₃ | CH₃ | (CH₂)₃ | — | — | 417 |
| 487 | 8-CH₃ | CH₃ | H | CH₃ | (CH₂)₃ | 2-F | 5-CF₃ | 483 |
| 488 | 8-Cl | H | CH₃ | CH₃ | (CH₂)₃ | 3-Cl | 4-Cl | 485/487 |
| 489 | 8-Cl | H | CH₃ | CH₃ | (CH₂)₃ | 3-F | 4-CF₃ | 503 |
| 490 | 8-Cl | H | CH₃ | CH₃ | (CH₂)₃ | 2-Cl | 3-Cl | 485/487 |
| 491 | 8-Cl | H | CH₃ | CH₃ | (CH₂)₃ | 3-F | 5-CF₃ | 503 |
| 491a | 8-CH₃ | H | H | H | (CH₂)₃ | 2-F | 5-CF₃ | 455 |

*MS = experimental mass spectrometry data; [M + H]

Table 11 depicts compounds of Formula Ia:

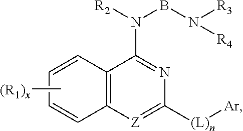

wherein Z is N, n is 0, and Ar is

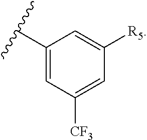

TABLE 11

| No. | R₁ | R₂ | R₃ | R₄ | B | R₅ | MS* |
|---|---|---|---|---|---|---|---|
| 492 | 8-CH₃ | H | ethyl | ethyl | CH(CH₃)(CH₂)₃ | Ph | 521 |
| 493 | 8-CH₃ | H | ethyl | ethyl | CH(CH₃)(CH₂)₃ | 3-CF₃—Ph | 589 |
| 494 | 8-CH₃ | H | CH₃ | CH₃ | (CH₂)₃ | 3-CF₃—Ph | 533 |
| 495 | 8-CH₃ | H | ethyl | ethyl | CH(CH₃)(CH₂)₃ | 4-CF₃—Ph | 589 |
| 496 | 8-CH₃ | H | CH₃ | CH₃ | (CH₂)₃ | 4-CF₃—Ph | 533 |
| 497 | 8-CH₃ | H | ethyl | ethyl | CH(CH₃)(CH₂)₃ | 3,4-(CH₂)₃—Ph | 549 |
| 498 | 8-CH₃ | H | CH₃ | CH₃ | (CH₂)₃ | 3,4-(CH₂)₃—Ph | 493 |
| 499 | 8-CH₃ | H | CH₃ | CH₃ | (CH₂)₃ | — | 389 |
| 500 | 8-CH₃ | H | ethyl | ethyl | CH(CH₃)(CH₂)₃ | morpholino | 530 |
| 501 | 8-CH₃ | H | CH₃ | CH₃ | (CH₂)₃ | morpholino | 474 |
| 502 | 8-CH₃ | CH₃ | CH₃ | CH₃ | (CH₂)₃ | Br | 481/483 |
| 503 | 8-F | H | CH₃ | CH₃ | (CH₂)₃ | Br | 471/473 |
| 504 | 8-CH₃ | H | CH₃ | CH₃ | (CH₂)₃ | OCH₃ | 419 |
| 505 | 8-CF3 | H | CH₃ | CH₃ | (CH₂)₃ | Br | 521/523 |
| 506 | 8-CH₃ | H | CH₃ | CH₃ | (CH₂)₃ | Cl | 423 |
| 507 | 8-CH₃ | H | CH₃ | CH₃ | (CH₂)₃ | CH₃ | 403 |
| 508 | 8-CH₃ | H | CH₃ | CH₃ | (CH₂)₃ | F | 407 |
| 509 | 8-CH₃ | H | CH₃ | CH₃ | (CH₂)₃ | 2-CF₃—Ph | 533 |
| 510 | 8-CH₃ | H | CH₃ | CH₃ | (CH₂)₃ | 3-pyridyl | 466 |
| 511 | 7-Cl, 8-CH₃ | H | CH₃ | CH₃ | (CH₂)₃ | Br | 501/503 |
| 512 | 7-Br-8-CH₃ | H | CH₃ | CH₃ | (CH₂)₃ | Br | 547 |
| 513 | 8-CH₃ | H | CH₃ | CH₃ | (CH₂)₃ | 2-Cl—Ph | 499 |
| 514 | 8-CF3 | H | CH₃ | CH₃ | (CH₂)₃ | Ph | 519 |
| 515 | 6-Br | H | CH₃ | CH₃ | (CH₂)₃ | Br | 533 |
| 516 | 6-CH₃ | H | CH₃ | CH₃ | (CH₂)₃ | Br | 467/469 |
| 517 | 8-CH₃ | H | CH₃ | CH₃ | (CH2)₄ | Br | 481/483 |
| 518 | 7-SONH₂ | H | CH₃ | CH₃ | (CH₂)₃ | Br | 532/534 |
| 519 | 7-SONH₂ | H | CH₃ | CH₃ | (CH₂)₃ | Ph | 530 |
| 520 | 5,8-(CH₃)₂ | H | CH₃ | CH₃ | (CH₂)₃ | Br | 481/483 |
| 521 | 8-CH₃ | H | CH₃ | CH₃ | (CH₂)₃ | cyclohexyl | 471 |
| 522 | 5,8-(CH₃)₂ | H | CH₃ | CH₃ | (CH₂)₃ | Ph | 479 |
| 523 | 8-isopropyl | H | CH₃ | CH₃ | (CH₂)₃ | Br | 495/497 |
| 524 | 8-isopropyl | H | CH₃ | CH₃ | (CH₂)₃ | Ph | 493 |
| 524a | 8-CH₃ | H | H | H | (CH₂)₃ | Br | 439/441 |
| 524b | 8-CH₃ | H | CH₃ | CH₃ | (CH₂)₃ | 2-pyridyl | 466 |
| 524c | 8-CH₃ | H | CH₃ | CH₃ | (CH₂)₃ | 5-pyrmidinyl | 467 |
| 524d | 7-Ph, 8-CH₃ | H | CH₃ | CH₃ | (CH₂)₃ | Br | 543/545 |
| 524e | 7-Ph, 8-CH₃ | H | CH₃ | CH₃ | (CH₂)₃ | Ph | 541 |
| 524f | 8-CH₃ | H | CH₃ | CH₃ | (CH₂)₂ | Ph | 451 |
| 524g | 6-Br, 8-CH₃ | H | CH₃ | CH₃ | (CH₂)₃ | Br | 547 |
| 524h | 8-CH₃ | H | CH₃ | CH₃ | (CH₂)₃ | 5-(1H-pyrazolo) | 455 |
| 524i | 8-CH₃ | H | H | H | (CH₂)₃ | Ph | 437 |
| 524j | 8-isopropyl | CH₃ | H | CH₃ | (CH₂)₃ | Ph | 493 |
| 524k | 8-CH₃ | CH₃ | H | CH₃ | (CH₂)₃ | 2-Cl—Ph | 499 |
| 524m | 8-CH₃ | H | H | H | (CH₂)₃ | 2-Cl—Ph | 471 |
| 524n | 8-CH₃ | CH₃ | H | H | (CH₂)₃ | Br | 453/455 |

*MS = experimental mass spectrometry data; [M + H]

Exemplary compounds of formula IIIa:

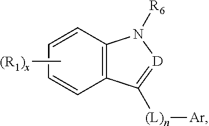

wherein D is N, n is 0, and Ar is

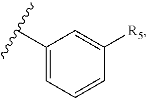

are listed in Table 12.

TABLE 12

| No. | R₁ | R₆ | R₅ | MS* |
|---|---|---|---|---|
| 525 | 5-Cl | H | 3-CF₃—Ph | 373 |
| 526 | 6-Cl | H | Ph | 305 |
| 527 | 6-CF₃ | H | 3,4-(Cl)₂—Ph | 407 |
| 528 | 6-CF₃ | H | (3-F-4-CF₃)—Ph | 425 |
| 529 | 6-CF₃ | H | 3-CF₃—Ph | 407 |
| 530 | 6-CF₃ | H | 3,5-(Cl)₂—Ph | 407 |
| 531 | 6-CF₃ | H | (2-F-4-CF₃)—Ph | 425 |
| 532 | 5-Cl | H | 3,4-(Cl)₂—Ph | 373 |
| 533 | 5-Cl | 3-chlorobenzyl | Ph | 429 |

*MS = experimental mass spectrometry data; [M + H]

Exemplary compounds of formula IIIa:

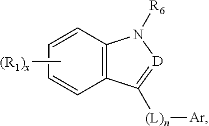

wherein D is N and n is 0, are listed in Table 13.

TABLE 13

| No. | R₁ | R₆ | Ar | MS* |
|---|---|---|---|---|
| 534 | 6-F | H | 3-Cl—Ph | 247 |
| 535 | 6-F | H | 3-CH₃—Ph | 227 |
| 536 | 6-CF₃ | H | 3-CF₃—Ph | 331 |
| 537 | 6-F | H | 3-CF₃ | 231 |
| 538 | 6-F | H | 3-CN—Ph | 238 |
| 539 | 5-Cl | H | (3-F-4-CF₃)—Ph | 315 |
| 540 | 6-CF₃ | H | 2-naphthyl | 313 |
| 541 | 6-CF₃ | H | 3,4-(Cl)₂—Ph | 331 |
| 542 | 6-CF₃ | 4-chlorobenzyl | 3,4-(Cl)₂—Ph | 455 |
| 543 | 6-CF₃ | H | 2,3-(Cl)₂—Ph | 331 |
| 544 | 6-CF₃ | H | (2-F-4-CF₃)—Ph | 349 |
| 545 | 6-CF₃ | 3-chlorobenzyl | 3,4-(Cl)₂—Ph | 455 |
| 546 | 6-CF₃ | H | (3-F-4-CF₃)—Ph | 349 |
| 547 | 6-CF₃ | H | 2-CF₃—Ph | 331 |
| 548 | 6-CF₃ | H | (3-Br-5-CF₃)—Ph | 409 |
| 549 | 6-CF₃ | H | (4-F-3-CF₃)—Ph | 349 |
| 550 | 6-CF₃ | H | (3-F-5-CF₃)—Ph | 349 |
| 551 | 6-CF₃ | H | (3-Cl-5-CF₃)—Ph | 365 |
| 552 | 6-CF₃ | H | (3-MeO-5-CF₃)—Ph | 361 |
| 553 | 6-CF₃ | H | (3-MeOC(O)- | 389 |
| | | | 5-CF₃)—Ph | |
| 554 | 6-CF₃ | H | (3-NH₂-5-CF₃)—Ph | 346 |
| 555 | 5-CF₃ | H | 3,4-(Cl)₂—Ph | 331 |
| 556 | 6-CF₃ | H | 3,5-(Cl)₂—Ph | 277 |
| 557 | 5-CF₃ | H | 3,4-(Cl)₂—Ph | 331 |
| 558 | 6-CF₃ | 3,4-dichlorobenzoyl | 3,4-(Cl)₂—Ph | 503 |
| 559 | 6-Br | H | 3,4-(Cl)₂—Ph | 341 |
| 559a | 6-CF₃ | N,N-dimethylpropyl | 3,4-(Cl)₂—Ph | 416 |
| 559b | 6-CF₃ | dimethylglycinoyl | 3,4-(Cl)₂—Ph | 416 |
| 559c | 6-CF₃ | N,N-dimethylethyl | 3,4-(Cl)₂—Ph | 402 |
| 559d | 6-CF₃ | 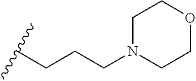 [SMILES: *CCCN1CCOCC1] | 3,4-(Cl)₂—Ph | 458 |

*MS = experimental mass spectrometry data; [M + H]

Table 14 depicts compounds of Formula Ia:

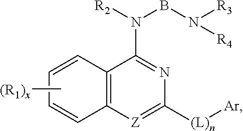

wherein Z is N, n is 0, and Ar is

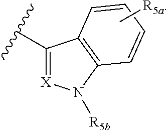

TABLE 14

| No. | R₁ | R₂ | R₃ | R₄ | B | R₅ₐ | R_{5b} | X | MS* |
|---|---|---|---|---|---|---|---|---|---|
| 560 | 8-CH₃ | H | ethyl | ethyl | CH(CH₃)(CH₂)₃ | 7-Cl | CH₃ | C | 464 |
| 561 | 8-CH₃ | H | CH₃ | CH₃ | (CH₂)₃ | 7-Cl | CH₃ | C | 408 |
| 562 | H | H | ethyl | ethyl | CH(CH₃)(CH₂)₃ | 7-Cl | benzyl | C | 526 |
| 563 | H | H | CH₃ | CH₃ | (CH₂)₃ | 7-Cl | benzyl | C | 470 |
| 564 | 8-F | H | ethyl | ethyl | CH(CH₃)(CH₂)₃ | 7-Cl | benzyl | C | 544 |
| 565 | 8-F | H | CH₃ | CH₃ | (CH₂)₃ | 7-Cl | benzyl | C | 488 |
| 566 | 8-Cl | H | ethyl | ethyl | CH(CH₃)(CH₂)₃ | 7-Cl | benzyl | C | 560 |
| 567 | 8-Cl | H | CH₃ | CH₃ | (CH₂)₃ | 7-Cl | benzyl | C | 504 |
| 568 | 8-Br | H | ethyl | ethyl | CH(CH₃)(CH₂)₃ | 7-Cl | benzyl | C | 604/606 |
| 569 | 8-Br | H | CH₃ | CH₃ | (CH₂)₃ | 7-Cl | benzyl | C | 548/550 |
| 570 | 8-(4-pyridyl) | H | ethyl | ethyl | CH(CH₃)(CH₂)₃ | 7-Cl | benzyl | C | 603 |
| 571 | 8-(4-pyridyl) | H | CH₃ | CH₃ | (CH₂)₃ | 7-Cl | benzyl | C | 547 |
| 572 | 8-CH₃ | H | ethyl | ethyl | CH(CH₃)(CH₂)₃ | 7-Cl | (4-CH₃)benzyl | C | 554 |
| 573 | 8-CH₃ | H | CH₃ | CH₃ | (CH₂)₃ | 7-Cl | (4-CH₃)benzyl | C | 498 |
| 574 | 8-CH₃ | H | ethyl | ethyl | CH(CH₃)(CH₂)₃ | 7-Cl | (3-CH₃)benzyl | C | 554 |
| 575 | 8-CH₃ | H | CH₃ | CH₃ | (CH₂)₃ | 7-Cl | (3-CH₃)benzyl | C | 498 |
| 576 | 8-CH₃ | H | ethyl | ethyl | CH(CH₃)(CH₂)₃ | 7-Cl | (4-Cl)benzyl | C | 574 |
| 577 | 8-CH₃ | H | CH₃ | CH₃ | (CH₂)₃ | 7-Cl | (4-Cl)benzyl | C | 518 |
| 578 | 8-CH₃ | H | ethyl | ethyl | CH(CH₃)(CH₂)₃ | 7-Cl | (3-Cl)benzyl | C | 574 |
| 579 | 8-CH₃ | H | CH₃ | CH₃ | (CH₂)₃ | 7-Cl | (3-Cl)benzyl | C | 518 |
| 580 | 8-CH₃ | H | H | CH₃ | (CH₂)₃ | 7-Cl | benzyl | C | 470 |
| 581 | 8-CH₃ | H | H | ethyl | CH(CH₃)(CH₂)₃ | 7-Cl | benzyl | C | 512 |
| 582 | 8-CH₃ | CH₃ | CH₃ | CH₃ | (CH₂)₃ | 7-Cl | (4-Cl)benzyl | C | 532 |
| 583 | 8-CH₃ | H | H | CH₃ | (CH₂)₃ | 7-Cl | (4-Cl)benzyl | C | 504 |
| 584 | 7-Cl | H | ethyl | ethyl | CH(CH₃)(CH₂)₃ | 7-Cl | benzyl | C | 560 |
| 585 | 7-Cl | H | CH₃ | CH₃ | (CH₂)₃ | 7-Cl | benzyl | C | 504 |
| 586 | 8-CH₃ | H | CH₃ | CH₃ | (CH₂)₃ | 6-Cl | benzyl | C | 484 |
| 587 | 8-CH₃ | H | CH₃ | CH₃ | (CH₂)₃ | 5-Cl | benzyl | C | 484 |
| 588 | 8-CH₃ | H | CH₃ | CH₃ | (CH₂)₃ | 4-Cl | benzyl | C | 484 |
| 589 | 8-CH₃ | H | CH₃ | CH₃ | (CH₂)₃ | 6,7-(Cl)₂ | benzyl | C | 518 |
| 590 | 8-CH₃ | H | CH₃ | CH₃ | (CH₂)₃ | 7-Cl | benzyl | N | 504 |

*MS = experimental mass spectrometry data; [M + H]

Table 15 depicts compounds of Formula Ia:

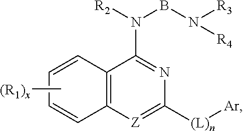

wherein n is 0.

TABLE 15

| No. | R₁ | R₂ | R₃ | R₄ | B | Ar | MS* |
|---|---|---|---|---|---|---|---|
| 591 | 8- Cl | H | CH₃ | CH₃ | (CH₂)₃ | 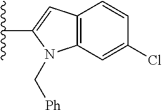 [SMILES: *c1cc2ccc(Cl)cc2n1Cc1ccccc1] | 504 |
| 592 | 8- Cl | H | CH₃ | CH₃ | (CH₂)₃ | 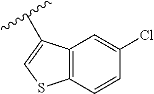 [SMILES: *c1csc2ccc(Cl)cc12] | 431 |

*MS = experimental mass spectrometry data; [M + H]

### EXAMPLES

### Example 1

### Synthesis of Compounds

Using the synthetic schemes shown above, the following compounds were prepared.

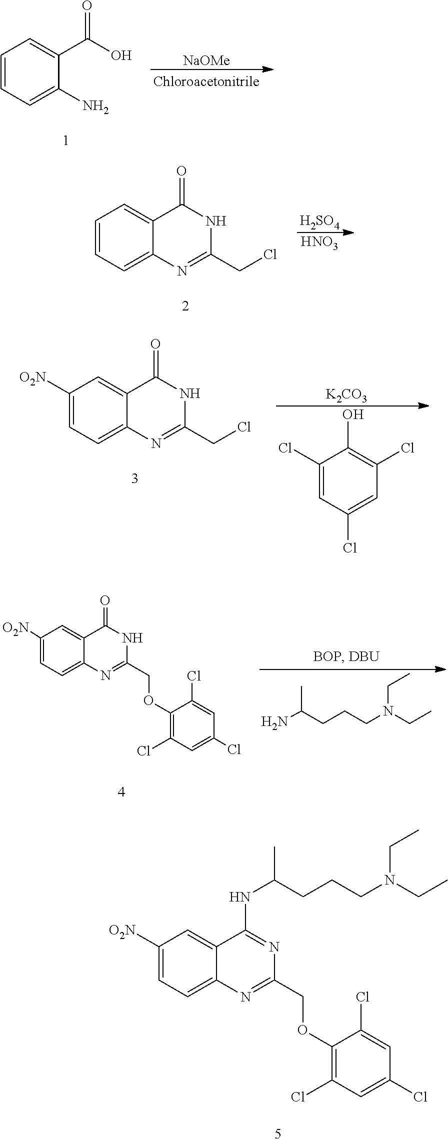

A 250 mL round bottom flask was equipped with a stir bar and addition funnel. 1 (Aldrich, 1.37 g, 10 mmol, 1 eq) was added to the addition funnel, and the system was evacuated and back-filled with Ar (×3). Anhydrous MeOH was added to the flask (4 mL) and addition funnel (50 mL). NaOMe (0.5 M in MeOH, 4 mL, 2 mmol, 0.2 eq) was added to the reaction flask. Chloroacetonitrile (1.9 mL, 2.26 g, 30 mmol, 3 eq) was added dropwise to the resulting solution with stirring. After approximately 40 min the solution of 1 was added dropwise with stirring. After stirring overnight a slurry had formed. The solids were collected via vacuum filtration. The filter cake was rinsed with MeOH (×2), water (×2), MeOH (×1) and air dried. 1.37 g (7.0 mmol, 70% yield) of 2 was collected as a white solid. Mass spectrum (ESI⁺): m/z=195 [M+1].

A 25 mL pear-shaped flask with stir bar was charged with conc. H₂SO₄ (0.7 mL) and fuming HNO₃ (0.7 mL). 2 (0.5 g, 2.5 mmol) was added to the colorless solution with stirring. After 4 h the resulting yellow solution poured onto ice. The resulting mixture was vacuum filtered to collect the solids. The filter cake was rinsed with water (×2) and air dried. 0.532 g (2.2 mmol, 89% yield) of 3 was collected as a white solid. Mass spectrum (ESI⁺): m/z=240 [M+1].

A 50 mL round bottom flask with stir bar was charged with 2,4,6-trichlorophenol (Aldrich, 0.378 g, 1.9 mmol, 1.1 eq). The flask was evacuated and back-filled with Ar (×3). Anhydrous DMF (4 mL) was added followed by K₂CO₃ (0.265 g, 1.9 mmol, 1.1 eq). The resulting mixture was stirred at room temperature for 10 min before cooling to 0° C. 3 (0.417 g, 1.7 mmol, 1 eq) was added, and the reaction was stirred at 0° C. to room temperature overnight. The reaction was quenched with water and extracted with EtOAc (×3). The combined organics were dried over MgSO₄. The solids were filtered off, and the volatiles were removed via rotary evaporation. DCM was added to the resulting residue. After stirring for 30 min the mixture was plug filtered through silica gel, rinsing repeatedly with DCM. The filtrate was concentrated via rotary evaporation yielding 0.208 g (0.52 mmol, 30% yield) of 4 as an off-white solid.

A 50 mL round bottom flask with stir bar was charged with 4 (0.06 g, 0.15 mmol, 1 eq). The flask was evacuated and back-filled with Ar (×3). Anhydrous CH₃CN (5 mL) was added followed by (benzotriazol-1-yloxy)tris(dimethylamino)phosphoniumhexafluorophosphate (0.086 g, 0.19 mmol, 1.3 eq). The resulting stirred suspension was treated with 1,8-diazabicyclo[5.4.0]undec-7-ene (0.034 mL, 0.034 g, 0.22 mmol, 1.5 eq) and a homogeneous solution rapidly formed. After 10 min 2-amino-5-diethylaminopentane (0.044 mL, 0.036 g, 0.22 mmol, 1.5 eq) was added. The reaction was stirred at temperature for 10 min before heating to 60° C. After 2 h the reaction was cooled to room temperature, and the volatiles were removed via rotary evaporation. The residue was partitioned between EtOAc/water, and the layers were separated. The organic layer was washed with water (×2), brine (×1), and dried over MgSO₄. The solids were filtered off, and the volatiles were removed via rotary evaporation. Purification via flash chromatography on silica gel eluting with 0-10% MeOH in DCM yielded 0.0397 g (0.073 mmol, 49% yield) of 5 as a sticky yellow semisolid. ¹H NMR (CDCl₃, 300 MHz) δ 8.81 (s, 1H), 8.49-8.45 (m, 1H), 7.95 (d, J=9.0 Hz, 1H), 7.81 (d, J=6.3 Hz, 1H), 7.32 (s, 2H), 5.17 (s, 2H), 4.44-4.42 (m, 1H), 2.76-2.55 (m, 6H), 1.83-1.60 (m, 4H), 1.30 (d, J=6.6 Hz, 3H), 1.09-1.05 (m, 6H). Mass spectrum (ESI⁺): m/z=540 [M+1].

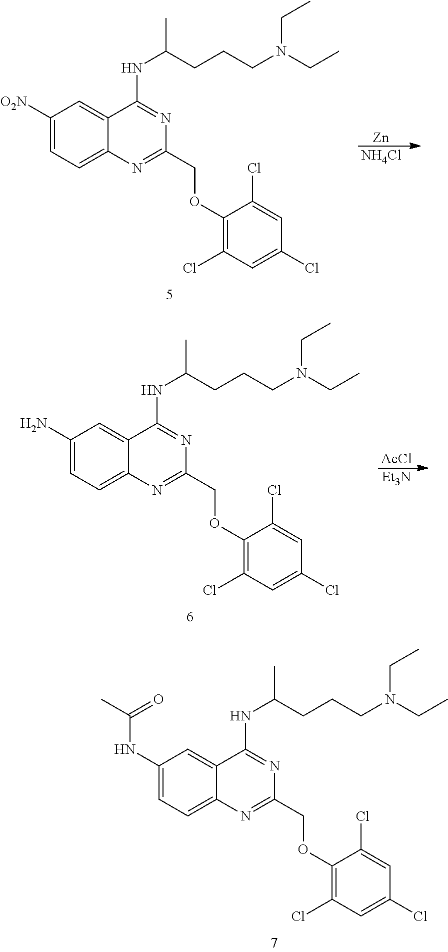

A 50 ml round bottom flask with stir bar was charged with 5 (0.075 g, 0.14 mmol, 1 eq). THF (2 mL) was added followed by NH₄Cl (aq, saturated, 1 mL). The resulting mixture was treated with Zn (0.0453 g, 0.69 mmol, 5 eq) with vigorous stirring. After 4 h the mixture was diluted with EtOAc and filtered through Celite. The mixture was diluted with water, and the layers were separated. The organic layer was washed with brine (×1) and dried over Na₂SO₄. The solids were filtered off, and the volatiles were removed via rotary evaporation. 0.043 g (0.084 mmol, 60% yield) of crude 6 was isolated and carried forward without purification. Mass spectrum (ESI⁺): m/z=510 [M+1].

A 25 mL round bottom flask with stir bar was charged with 6 (0.043 g, 0.084 mmol, 1 eq). The flask was evacuated and back-filled with Ar (×3). Anhydrous DCM (5 mL) was added, and the resulting solution was cooled to 0° C. with stirring. Et₃N (0.02 mL, 0.0129 g, 0.127 mmol, 1.5 eq) was added followed by acetyl chloride (6 μL, 6.6 mg, 0.084 mmol, 1 eq) 5 min later. After 30 min the cooling bath was removed, and the reaction was allow to warm to room temperature. After stirring at temperature for 4 h the reaction was quenched with NaHCO₃ (aq, saturated). Additional DCM was added, and the layers were separated. The organic layer was dried over Na₂SO₄. The solids were filtered off, and the volatiles were removed via rotary evaporation. Purification via flash chromatography on silica gel eluting with 0-20% MeOH in DCM yielded the crude product. The crude was purified by plug filtration through silica gel eluting with MeOH/NH₃ (aq, sat)/EtOAc (10:1.5:88.5) yielding 0.0108 g (0.02 mmol, 23% yield) of 7. ¹H NMR (CDCl₃, 300 MHz) δ 8.50 (s, 1H), 8.41 (s, 1H), 7.75 (d, J=8.7 Hz, 1H), 7.48 (d, J=9.3 Hz, 1H), 7.29 (s, 2H), 5.78 (d, J=7.8 Hz, 1H), 5.14 (s, 2H), 4.44-4.39 (m, 1H), 2.57-2.45 (m, 6H), 2.24 (s, 3H), 1.67-1.59 (m, 4H), 1.25 (d, J=6.6 Hz, 3H), 1.03-0.98 (m, 6H). Mass spectrum (ESI⁺): m/z=552 [M+1].

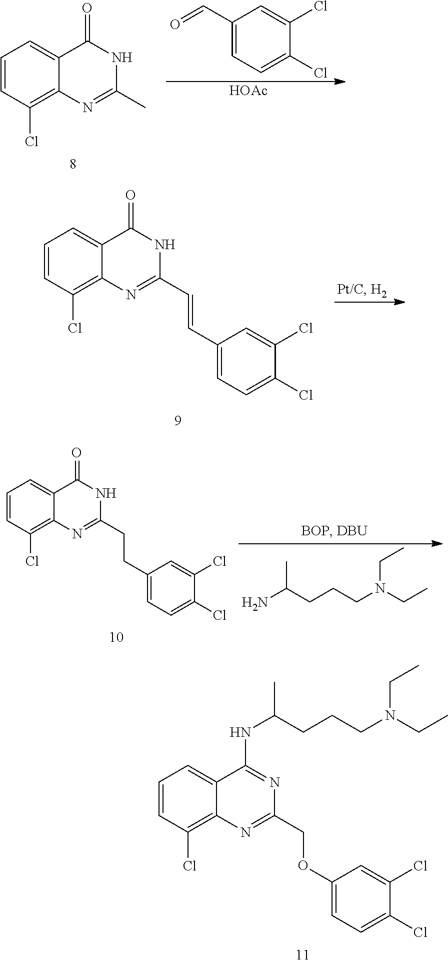

A 20 mL screw-top vial with stir bar was charged with 8 (Combi-Blocks, 0.5 g, 2.57 mmol, 1 eq) and 3,4-dichlorobenzaldehyde (Combi-Blocks, 0.450 g, 2.57 mmol, 1 eq). HOAc (5 mL) was added, and the vial was closed. The resulting mixture was heated to 120° C. with stirring overnight. After cooling to room temperature the mixture was diluted with hexanes, and the solids collected via vacuum filtration. The filter cake was rinsed with hexanes (×3) and dried under vacuum. 0.816 g (2.3 mmol, 90% yield) of 9 was collected as a pale yellow solid. Mass spectrum (ESI⁺): m/z=351 [M+1].

A 100 mL round bottom flask with stir bar was charged with 9 (0.250 g, 0.71 mmol, 1 eq) and 5% Pt/C (0.080 g, 32% by wt.). EtOAc (50 mL) was added, and the resulting mixture was stirred vigorously under H₂ (balloon pressure) overnight. The mixture was filtered through Celite, rinsing with 1:1 EtOAc/DCM (600 mL). Silica gel (2.5 g) was added to the filtrate, and the volatiles were removed via rotary evaporation. Purification via flash chromatography (40 gram column) on silica gel eluting with 0-30% EtOAc in DCM yielded 0.091 g (0.26 mmol, 36% yield) of 10 as a white solid. Mass spectrum (ESI⁺): m/z=353 [M+1].

A 20 mL screw-top vial with stir bar was charged with 10 (0.051 g, 0.14 mmol, 1 eq) and BOP (0.0829 g, 0.19 mmol, 1.3 eq). The vial was evacuated and back-filled with Ar (×3). Anhydrous DMF (5 mL) was added. After stirring for 5 min DBU (0.032 mL, 0.033 g, 0.21 mmol, 1.5 eq) was added. A homogeneous solution gradually formed. After 20 min 2-amino-5-diethylaminopentane (0.042 mL, 0.034 g, 0.21 mmol, 1.5 eq) was added. The reaction was stirred at temperature for 30 min before heating to 50° C. After 1 h the reaction was cooled to room temperature and poured into water. The resulting mixture was extracted with EtOAc (×1), and the layers were separated. The organic layer was washed with water (×2), brine (×1), and dried over Na₂SO₄. The solids were filtered off, and the volatiles were removed via rotary evaporation. Purification via flash chromatography on silica gel eluting with 0-15% MeOH in DCM yielded 0.0457 g (0.093 mmol, 66% yield) of 11 as an oil. ¹H NMR (CDCl₃, 300 MHz) δ 7.65-7.62 (m, 1H), 7.55-7.47 (m, 2H), 7.38-7.30 (m, 3H), 7.11-7.08 (m, 1H), 4.49-4.43 (m, 1H), 3.18-3.04 (m, 4H), 2.56-2.44 (m, 6H), 1.67-1.55 (m, 4H), 1.30 (d, J=6.6 Hz, 3H), 1.03-0.98 (m, 6H). Mass spectrum (ESI⁺): m/z=493 [M+1].

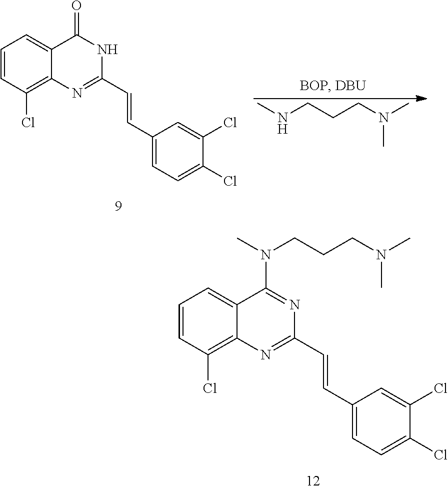

Conversion of 9 to 12 was carried according to the method described for the synthesis of 11 above using N,N,N′-trimethyl-1,3-propanediamine (Aldrich). Purification via flash chromatography on silica gel eluting with 0-20% MeOH in DCM yielded 12 in 30% yield as a yellow oil. ¹H NMR (CDCl₃, 300 MHz) δ 7.83 (d, J=15.9 Hz, 1H), 7.71-7.68 (m, 2H), 7.57-7.52 (m, 1H), 7.45-7.36 (m, 3H), 7.15 (d, J=15.9 Hz, 1H), 3.72 (broad s, 2H), 3.19 (s, 3H), 2.33-2.28 (m, 2H), 2.19 (s, 6H), 1.92-1.88 (m, 2H). Mass spectrum (ESI⁺): m/z=449 [M+1].

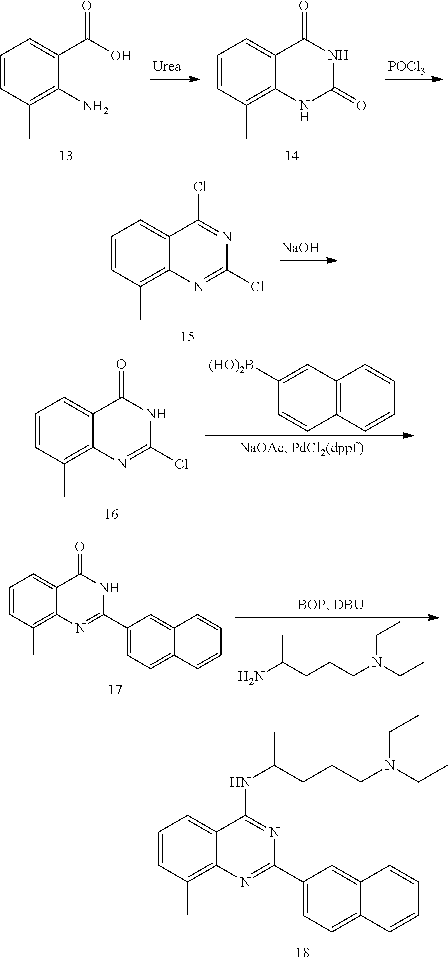

A 100 mL pear-shaped flask with stir bar was charged with 13 (Combi-Blocks, 2.0 g, 13.2 mmol, 1 eq) and urea (7.94 g, 132 mmol, 10 eq). The flask was evacuated and back-filled with Ar (×3). The resulting stirred mixture was heated to 150° C. overnight. After cooling to 100° C. water (40 mL) was added. The resulting hot mixture was filtered to collect the solids. The filter cake was triturated with MeOH (×3) and dried under vacuum. 2.82 g (16 mmol, 120% yield) of crude 14 was isolated as a pale solid. The crude was used in the next step without further purification.

A 50 mL round bottom flask with a reflux condenser and stir bar was charged with 14 (1.0 g, 5.7 mmol, 1 eq). The flask was evacuated and back-filled with Ar (×3). POCl₃ (2.6 mL, 4.35 g, 28.3 mmol, 5 eq) was added followed by DIPEA (2.0 mL, 1.47 g, 11.3 mmol, 2 eq). The resulting mixture was heated to reflux with stirring. After 5 h the reaction was cooled to room temperature and poured onto ice with vigorous stirring. The resulting suspension was cooled to 0° C. and adjusted to pH≈7-8 with NH₄OH (aq, concentrated). The solids were collected via vacuum filtration. The solids were suspended in DCM and plug filtered through silica gel, eluting with DCM. The filtrate was concentrated to dryness via rotary evaporation yielding 0.467 g (2.19 mmol, 38% yield) of 15 as a white solid.

A 100 mL round bottom flask with stir bar was charged with 15 (0.367 g, 1.72 mmol, 1 eq). THF (8 mL) was added. The resulting stirred solution was treated with 1N NaOH (4 mL, 4 mmol, 2.3 eq). After 2 h the volatiles were removed via rotary evaporation. The resulting mixture was adjusted to pH≈7 with 1N HCl. The solids were collected via vacuum filtration and air dried. 0.282 g (1.45 mmol, 84% yield) of 16 was isolated as a white solid. Mass spectrum (ESI⁺): m/z=195 [M+1].

A 25 mL pear-shaped flask with stir bar was charged with 16 (0.150 g, 0.77 mmol, 1 eq), naphthalene-2-boronic acid (Combi-Blocks, 0.199 g, 1.16 mmol, 1.5 eq), and NaOAc (0.283 g, 3.45 mmol, 4.47 eq). The flask was evacuated and back-filled with Ar (×3). EtOH/Toluene/water (2:5:2, 9 mL, sparged with Ar) was added. The resulting mixture was treated with PdCl₂(dppf) (0.113 g, 0.15 mmol, 0.2 eq) with stirring. The mixture was heated to 80° C. overnight. After cooling to room temperature the volatiles were removed via rotary evaporation. The resulting solids were triturated with DCM, and the solids were collect via vacuum filtration yielding 0.103 g (0.36 mmol, 47% yield) of 17. Mass spectrum (ESI⁺): m/z=287 [M+1].

Conversion of 17 to 18 was carried according to the method described for the synthesis of 11 above. Purification via flash chromatography on silica gel eluting with 0-15% MeOH in DCM yielded 18 in 79% yield as a colorless oil. ¹H NMR (CDCl₃, 300 MHz) δ 9.11 (s, 1H), 8.78-8.75 (m, 1H), 8.04-7.87 (m, 3H), 7.60-7.49 (m, 4H), 7.33-7.28 (m, 2H), 5.87-5.84 (m, 1H), 4.77-4.71 (m, 1H), 2.84 (s, 3H), 2.58-2.49 (m, 6H), 1.85-1.65 (m, 4H), 1.43 (d, J=6.3 Hz, 3H), 1.03-0.98 (m, 6H). Mass spectrum (ESI⁺): m/z=427 [M+1].

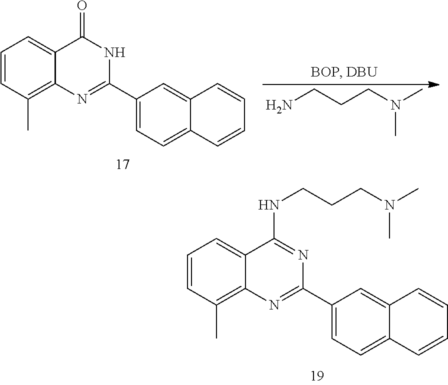

Conversion of 17 to 19 was carried according to the method described for the synthesis of 11 above using 3-dimethylamino-1-propylamine (Aldrich). Purification via flash chromatography on silica gel eluting with 0-20% MeOH in DCM yielded 19 in 79% yield as a white solid. ¹H NMR (CDCl₃, 300 MHz) δ 9.15 (s, 1H), 8.82-8.79 (m, 1H), 8.42 (broad s, 1H), 8.05-7.88 (m, 3H), 7.58-7.46 (m, 4H), 7.32-7.26 (m, 1H), 3.99-3.93 (m, 2H), 2.85 (s, 3H), 2.64-2.60 (m, 2H), 2.39 (s, 6H), 1.98-1.92 (m, 2H). Mass spectrum (ESI⁺): m/z=371 [M+1].

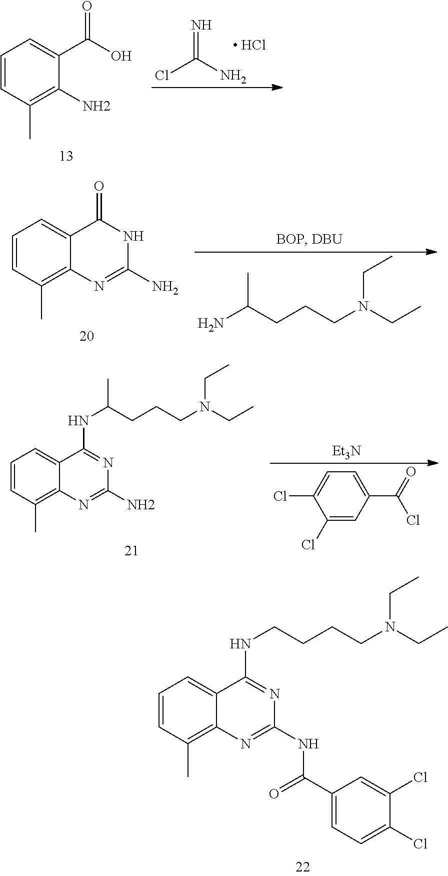

A 100 mL pear-shaped flask with stir bar was charged with 13 (1.32 g, 8.7 mmol, 1 eq), chloroformamidine hydrochloride (Oakwood Chemical, 2.01 g, 17.5 mmol, 2 eq), and dimethyl sulfone (4.11 g, 43.7 mmol, 5 eq). The flask was evacuated and back-filled with Ar (×3). Sulfolane (0.33 mL, 0.42 g, 3.5 mmol, 0.4 eq) was added. The resulting mixture was heated to 170° C. with stirring. After 1 h the hot reaction was quenched with water, and the resulting mixture was cooled to room temperature. The solids were collected via vacuum filtration. The solids were suspended in 1:1 MeOH/water, and the resulting mixture was filtered. The two filtrates were combined, and the pH was adjusted to 7-8 with NH₄OH (aq, conc.). A yellow solid formed and was collected via vacuum filtration. The solid was triturated with DCM (×3) and air dried yielding 1.34 g (7.6 mmol, 88% yield) of impure 20. The material was used without further purification. Mass spectrum (ESI⁺): m/z=176 [M+1].

A 20 mL vial with stir bar was charged with 20 (0.2 g, 1.14 mmol, 1 eq) and BOP (0.656 g, 1.48 mmol, 1.3 eq). The vial was evacuated and back-filled with Ar (×3). Anh. DMF (5 mL) was added, and the reaction was cooled to 0° C. with stirring. DBU (0.26 mL, 0.26 g, 1.71 mmol, 1.5 eq) was added followed by 2-amino-5-diethylaminopentane (0.33 mL, 0.27 g, 1.17 mmol, 1.5 eq) 10 min later. After 10 min the cooling bath was removed. The reaction was stirred at room temperature for 1 h before heating to 40° C. After 1 h the reaction was cooled to room temperature, diluted with water, and filtered through Celite to remove any insoluble material. The filtrate was extracted with EtOAc (×2). The combined organic fractions were washed with water (×2), brine (×1), and dried over Na₂SO₄. The solids were filtered off, and the volatiles were removed via rotary evaporation. The residue was partitioned between DCM and dilute HCl (pH≈2). The layers were separated. The aqueous layer was washed with DCM (×1). The aqueous layer was adjusted to pH≈7 with 1N NaOH. The mixture was extracted with EtOAc (×2). The combined EtOAc fractions were washed with water (×1), brine (×1), and dried over Na₂SO₄. The solids were filtered off, and the volatiles were removed via rotary evaporation. 0.056 g (0.18 mmol, 16% yield) of 21 was collected as a yellow oil. Mass spectrum (ESI⁺): m/z=316 [M+1].

A 25 ml round bottom flask with stir bar was charged with 21 (0.017 g, 0.05 mmol, 1 eq). The flask was evacuated and back-filled with Ar (×3). Anh. DCM (2 mL) was added. 3,4-dichlorobenzoyl chloride (0.0124 g, 0.06 mmol, 1.1 eq) and Et₃N (0.02 mL, 0.0145 g, 0.14 mmol, 2.7 eq) were added sequentially with stirring. After stirring overnight the volatiles were removed via rotary evaporation. The resulting crude was purified by prep TLC eluting with 13% MeOH in DCM followed by prep TLC eluting with 10:2:88 MeOH/NH₃ (aq, sat)/EtOAc. 0.015 g (0.029 mmol, 59% yield) of 22 was collected as a white sold. ¹H NMR (CDCl₃, 300 MHz) δ 8.45 (broad s, 1H), 8.14 (broad s, 1H), 7.61-7.48 (m, 3H), 7.29-7.24 (m, 2H), 4.77 (broad s, 1H), 268-2.46 (m, 9H), 1.76-1.59 (m, 4H), 1.32 (d, J=6.3 Hz, 3H), 1.24-1.04 (m, 6H). Mass spectrum (ESI⁺): m/z=488 [M+1].

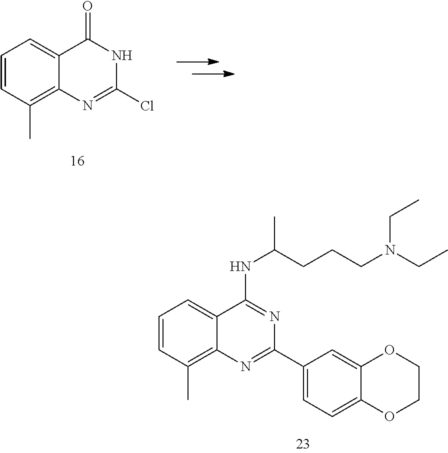 [SMILES: Cc1cccc2c(=O)[nH]c(Cl)nc12 ; CCN(CC)CCCC(C)Nc1nc(-c2ccc3c(c2)OCCO3)nc2c(C)cccc12]

Conversion of 16 to 23 was carried according to the method described for the synthesis of 18 above using 1,4-benzodioxane-6-boronic acid (Combi-Blocks). Purification via flash chromatography on silica gel eluting with 0-20% MeOH in DCM yielded 23 in 65% yield (final step) as a white solid. ¹H NMR (CDCl₃, 300 MHz) δ 8.17-8.10 (m, 2H), 7.54-7.48 (m, 2H), 7.27-7.22 (m, 1H), 6.95 (d, J=8.1 Hz, 1H), 5.76 (d, J=7.8 Hz, 1H), 4.69-4.60 (m, 1H), 4.32 (s, 4H), 2.75 (s, 3H), 2.56-2.45 (m, 6H), 1.79-1.58 (m, 4H), 1.36 (d, J=6.3 Hz, 3H), 1.02-0.98 (m, 6H). Mass spectrum (ESI⁺): m/z=435 [M+1].

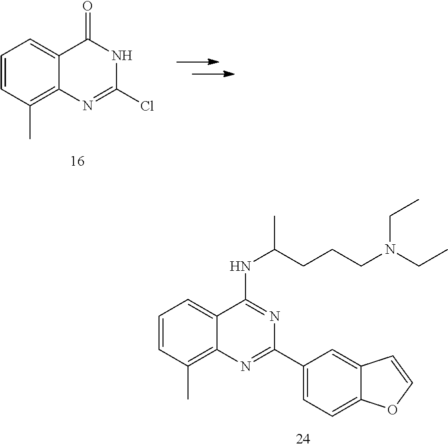 [SMILES: Cc1cccc2c(=O)[nH]c(Cl)nc12 ; CCN(CC)CCCC(C)Nc1nc(-c2ccc3occc3c2)nc2c(C)cccc12]

Conversion of 16 to 24 was carried according to the method described for the synthesis of 18 above using benzofuran-5-boronic acid (Combi-Blocks). Purification via flash chromatography on silica gel eluting with 0-20% MeOH in DCM yielded 24 in 73% yield (final step) as an amber oil. ¹H NMR (CDCl₃, 300 MHz) δ 8.89 (d, J=0.9 Hz, 1H), 8.68-8.64 (m, 1H), 7.65 (d, J=2.1 Hz, 1H), 7.59-7.52 (m, 3H), 7.29-7.24 (m, 1H), 6.88 (d, J=1.2 Hz, 1H), 5.82 (d, J=7.2 Hz, 1H), 4.75-4.67 (m, 1H), 2.81 (s, 3H), 2.57-2.48 (m, 6H), 1.83-1.66 (m, 4H), 1.41 (d, J=6.3 Hz, 3H), 1.03-0.98 (m, 6H). Mass spectrum (ESI⁺): m/z=417 [M+1].

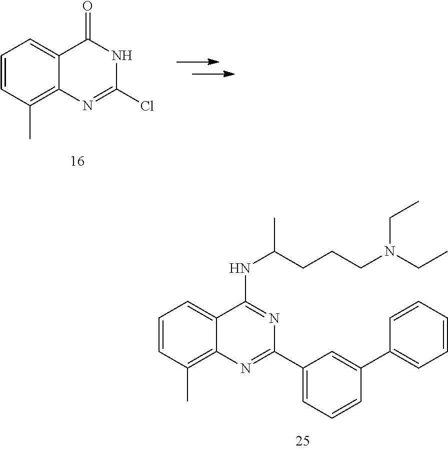 [SMILES: Cc1cccc2c(=O)[nH]c(Cl)nc12 ; CCN(CC)CCCC(C)Nc1nc(-c2cccc(-c3ccccc3)c2)nc2c(C)cccc12]

Conversion of 16 to 25 was carried according to the method described for the synthesis of 18 above using 3-biphenylboronic acid (Combi-Blocks). Purification via flash chromatography on silica gel eluting with 0-20% MeOH in DCM yielded 25 in 62% yield (final step) as an amber oil. ¹H NMR (CDCl₃, 300 MHz) δ 8.87 (s, 1H), 8.61 (d, J=7.8 Hz, 1H), 7.72-7.66 (m, 3H), 7.55-7.46 (m, 5H), 7.40-7.25 (m, 2H), 5.87 (d, J=7.2 Hz, 1H), 4.70-4.67 (m, 1H), 2.79 (s, 3H), 2.55-2.46 (m, 6H), 1.79-1.63 (m, 4H), 1.39 (d, J=6.6 Hz, 3H), 1.01-0.96 (m, 6H). Mass spectrum (ESI⁺): m/z=453 [M+1].

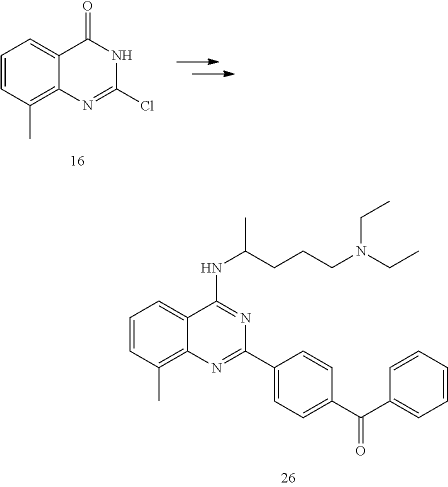 [SMILES: Cc1cccc2c(=O)[nH]c(Cl)nc12 ; CCN(CC)CCCC(C)Nc1nc(-c2ccc(C(=O)c3ccccc3)cc2)nc2c(C)cccc12]

Conversion of 16 to 26 was carried according to the method described for the synthesis of 18 above using 4-benzoylphenylboronic acid (Combi-Blocks). Purification via flash chromatography on silica gel eluting with 0-20% MeOH in DCM yielded 26 in 60% yield (final step) as an amber oil. ¹H NMR (CDCl₃, 300 MHz) δ 8.74 (d, J=8.1 Hz, 1H), 7.93-7.84 (m, 4H), 7.63-7.47 (m, 5H), 7.33-7.28 (m, 1H), 6.04 (d, J=7.5 Hz, 1H), 4.70-4.66 (m, 1H), 2.79 (s, 3H), 2.58-2.48 (m, 6H), 1.81-1.64 (m, 4H), 1.40 (d, J=6.3 Hz, 3H), 1.03-0.98 (m, 6H). Mass spectrum (ESI⁺): m/z=481 [M+1].

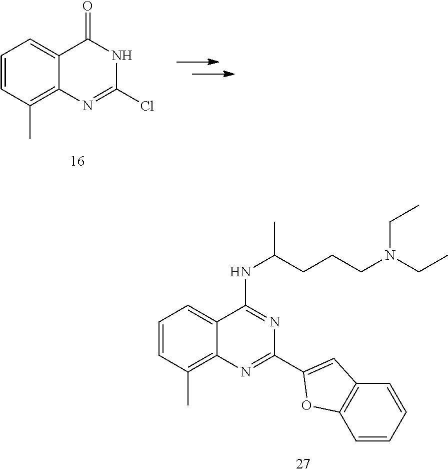 [SMILES: Cc1cccc2c(=O)[nH]c(Cl)nc12 ; CCN(CC)CCCC(C)Nc1nc(-c2cc3ccccc3o2)nc2c(C)cccc12]

Conversion of 16 to 27 was carried according to the method described for the synthesis of 18 above using benzofuran-2-boronic acid (Combi-Blocks). Purification via flash chromatography on silica gel eluting with 0-20% MeOH in DCM followed by prep TLC eluting with 1:10:89 NH₄OH (aq, sat)/MeOH/EtOAc yielded 27 in 5% yield (final step). ¹H NMR (CDCl₃, 300 MHz) δ 7.70-7.54 (m, 5H), 7.38-7.23 (m, 3H), 6.00 (d, J=7.5 Hz, 1H), 4.67-4.63 (m, 1H), 2.83 (s, 3H), 2.59-2.53 (m, 6H), 1.79-1.69 (m, 4H), 1.40 (d, J=6.3 Hz, 3H), 1.04-0.99 (m, 6H). Mass spectrum (ESI⁺): m/z=417 [M+1].

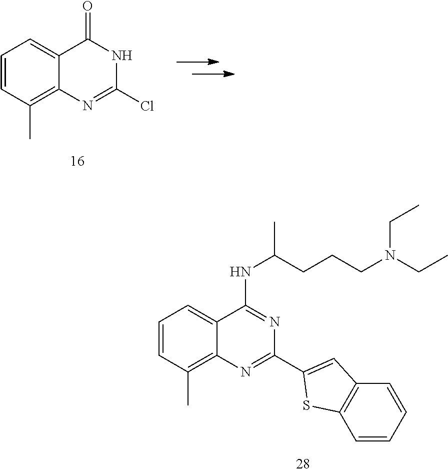 [SMILES: Cc1cccc2c(=O)[nH]c(Cl)nc12 ; CCN(CC)CCCC(C)Nc1nc(-c2cc3ccccc3s2)nc2c(C)cccc12]

Conversion of 16 to 28 was carried according to the method described for the synthesis of 18 above using benzo(b)thiophene-2-boronic acid (Combi-Blocks). Purification via flash chromatography on silica gel eluting with 0-20% MeOH in DCM followed by prep TLC eluting with 1:10:89 NH₄OH (aq, sat)/MeOH/EtOAc yielded 27 in 10% yield (final step). ¹H NMR (CDCl₃, 300 MHz) δ 8.29 (s, 1H), 7.88-7.84 (m, 2H), 7.58-7.51 (m, 2H), 7.37-7.29 (m, 2H), 5.95 (d, J=7.2 Hz, 1H), 4.66-4.62 (m, 1H), 2.77 (s, 3H), 2.60-2.50 (m, 6H), 1.82-1.69 (m, 4H), 1.41 (d, J=6.3 Hz, 3H), 1.04-1.00 (m, 6H). Mass spectrum (ESI⁺): m/z=433 [M+1].

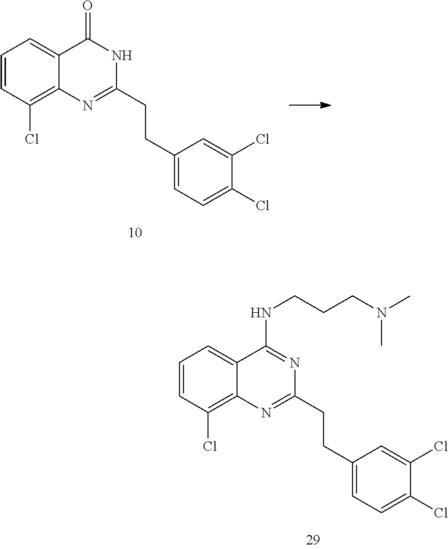 [SMILES: O=c1[nH]c(CCc2ccc(Cl)c(Cl)c2)nc2c(Cl)cccc12 ; CN(C)CCCNc1nc(CCc2ccc(Cl)c(Cl)c2)nc2c(Cl)cccc12]

Conversion of 10 to 29 was carried according to the method described for the synthesis of 11 above using 3-dimethylamino-1-propylamine. Purification via flash chromatography on silica gel eluting with 8-20% MeOH in DCM yielded 29 in 52% yield as a pale yellow solid. ¹H NMR (CDCl₃, 300 MHz) δ 8.31 (broad s, 1H), 7.64 (d, J=8.4 Hz, 1H), 7.53-7.50 (m, 1H), 7.39-7.30 (m, 2H), 7.12-7.09 (m, 1H), 3.73-3.67 (m, 2H), 3.17-3.05 (m, 4H), 2.50-2.46 (m, 2H), 2.28 (s, 6H), 1.90-1.82 (m, 2H). Mass spectrum (ESI⁺): m/z=437 [M+1].

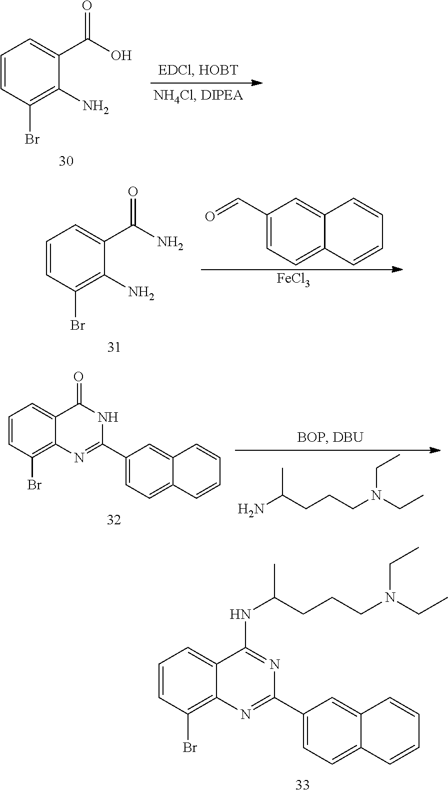

A 100 mL round bottom flask with stir bar was charged with 30 (Combi-Blocks, 0.500 g, 3.0 mmol, 1 eq) and NH₄Cl (0.486 g, 9.1 mmol, 3 eq). The flask was evacuated and back-filled with Ar (×3). Anh. DMF (15 mL) was added, and the mixture was cooled to 0° C. with stirring. 1-hydroxybenzotriazole hydrate (0.449 g, 3.3 mmol, 1.1 eq) was added followed by 1-(3-dimethylaminopropyl)-3-ethylcarbodiimide hydrochloride (0.636 g, 3.3 mmol, 1.1 eq) 5 min later. After 20 min N,N-diisopropylethylamine was added. The reaction was stirred at 0° C. to room temperature overnight. The reaction was diluted with NaHCO₃ (aq, sat) and extracted with EtOAc (×2). The combined EtOAc fractions were washed with water (×2), brine (×1), and dried over Na₂SO₄. The solids were filtered off, and the volatiles were removed via rotary evaporation. The resulting solid was triturated with 20% DCM/hexanes, and the solids were collect via vacuum filtration. 0.409 g (2.5 mmol, 83% yield) of 31 was collected as an off-white solid.

A sealed-tube vessel with stir bar was charged with 31 (0.2733 g, 1.3 mmol, 1 eq), 2-naphthaldehyde (Aldrich, 0.208 g, 1.33 mmol, 1.05 eq), and FeCl₃ (0.103 g, 0.64 mmol, 0.5 eq). CH₃CN/water (1:1, 3 mL) was added. The vessel was closed, and the mixture was heated to 170° C. overnight. After cooling to room temperature the reaction was diluted with water. The solids were collected via vacuum filtration. The filter cake was triturated with 1:1 EtOAc/DCM and air dried yielding 0.429 g (1.2 mmol, 94% yield) of 32 as a tan solid. Mass spectrum (ESI⁺): m/z=351 [M+1].

Conversion of 32 to 33 was carried according to the method described for the synthesis of 11 above. Purification via flash chromatography on silica gel eluting with 0-20% MeOH in DCM yielded 33 in 50% yield as an oil. ¹H NMR (CDCl₃, 300 MHz) δ 9.14 (s, 1H), 8.80-8.76 (m, 1H), 8.04-7.87 (m, 4H), 7.72 (d, J=8.4 Hz, 1H), 7.54-7.48 (m, 2H), 7.23-7.21 (m, 1H), 6.29 (d, J=7.2 Hz, 1H), 4.78-4.70 (m, 1H), 2.59-2.49 (m, 6H), 1.90-1.63 (m, 4H), 1.44 (d, J=6.3 Hz, 3H), 1.04-1.01 (m, 6H). Mass spectrum (ESI⁺): m/z=491, 493 [M+1].

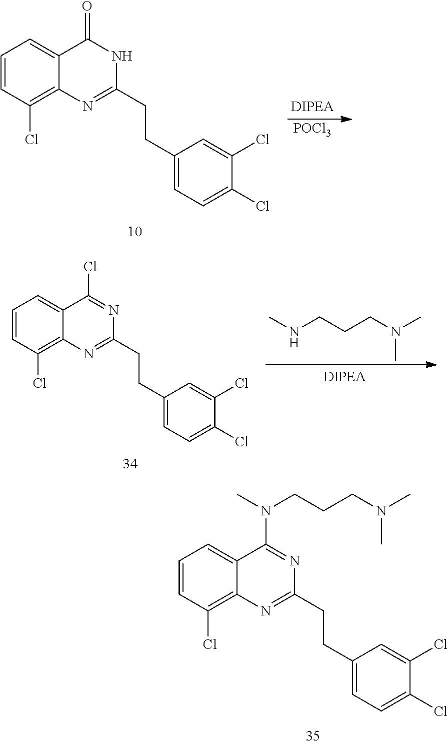

A 25 mL round bottom flask with stir bar was charged with 10 (0.01 g, 0.028 mmol, 1 eq). The flask was evacuated and back-filled with Ar (×3). Anh DCM (3 mL) was added. DIPEA (0.074 mL, 0.054 g, 0.42 mmol, 15 eq) and POCl₃ (0.026 mL, 0.043 g, 0.28 mmol, 10 eq) were added sequentially with stirring. A homogeneous solution gradually formed. The reaction was stirred overnight. The volatiles were removed via rotary evaporation yielding crude 34. The flask containing the crude 34 was evacuated and back-filled with Ar (×3). Anh DCM (2 mL) was added. The resulting stirred solution was treated with an DIPEA (0.049 mL, 0.037 g, 0.28 mmol, 10 eq) followed by N,N,N′-trimethyl-1,3-propanediamine (0.021 mL, 0.016 g, 0.14 mmol, 5 eq) 5 min later. The reaction was stirred overnight. The volatiles were removed via rotary evaporation. Purification via flash chromatography on silica gel eluting with 3-20% MeOH in DCM yielded 0.0050 g (0.011 mmol, 40% yield for 2 steps) of 35 as an oil. ¹H NMR (CDCl₃, 300 MHz) δ 7.65 (d, J=8.4 Hz, 1H), 7.55-7.50 (m, 1H), 7.37-7.30 (m, 3H), 7.11-7.08 (m, 1H), 3.65 (broad s, 2H), 3.13 (broad s, 7H), 2.34-2.32 (m, 2H), 2.23 (s, 6H), 1.91-1.86 (m, 2H). Mass spectrum (ESI⁺): m/z=451 [M+1].

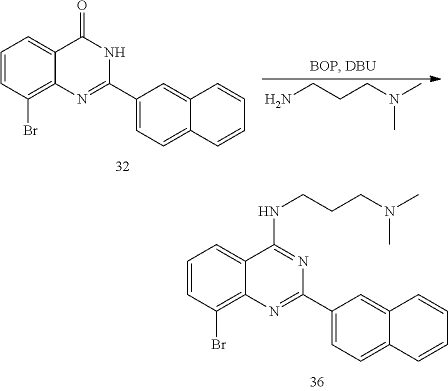

Conversion of 32 to 36 was carried according to the method described for the synthesis of 11 above using 3-dimethylamino-1-propylamine. Purification via flash chromatography on silica gel eluting with 0-20% MeOH in DCM yielded 36 in 23% yield as an oil. ¹H NMR (CDCl₃, 300 MHz) δ 9.14 (s, 1H), 8.80-8.71 (m, 2H), 8.04-7.77 (m, 5H), 7.52-7.49 (m, 2H), 7.28-7.23 (m, 1H), 4.00-3.99 (m, 2H), 2.80-2.77 (m, 2H), 2.51 (s, 6H), 2.08-2.05 (m, 2H). Mass spectrum (ESI⁺): m/z=435, 437 [M+1].

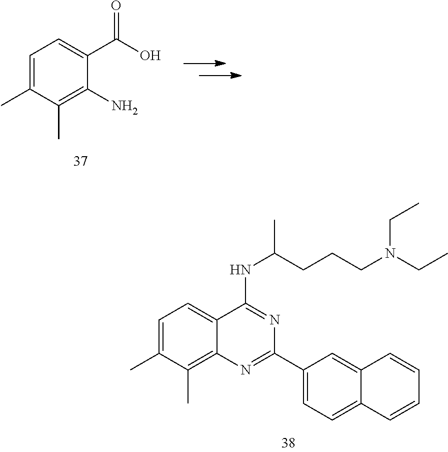 [SMILES: Cc1ccc(C(=O)O)c(N)c1C ; CCN(CC)CCCC(C)Nc1nc(-c2ccc3ccccc3c2)nc2c(C)c(C)ccc12]

Conversion of 37 (Combi-Blocks) to 38 was carried out in a manner similar to the method described for the synthesis of 33 above. Purification via flash chromatography on silica gel eluting with 0-20% MeOH in DCM yielded 38 in 50% yield (final step) as an oil. ¹H NMR (CDCl₃, 300 MHz) δ 9.11 (s, 1H), 8.79-8.76 (m, 1H), 8.04-7.87 (m, 3H), 7.52-7.46 (m, 3H), 7.25-7.20 (m, 1H), 5.81 (d, J=7.5 Hz, 1H), 4.77-4.73 (m, 1H), 2.80 (s, 3H), 2.58-2.47 (m, 9H), 1.92-1.66 (m, 4H), 1.42 (d, J=6.6 Hz, 3H), 1.03-0.98 (m, 6H). Mass spectrum (ESI⁺): m/z=441 [M+1].

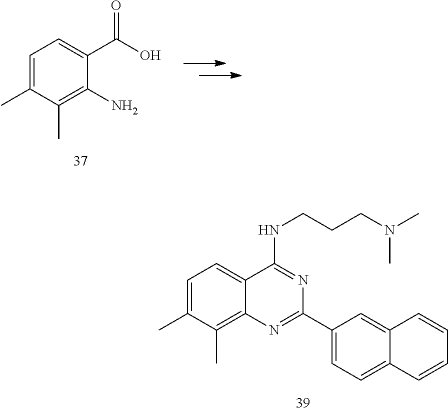 [SMILES: Cc1ccc(C(=O)O)c(N)c1C ; Cc1ccc2c(NCCCN(C)C)nc(-c3ccc4ccccc4c3)nc2c1C]

Conversion of 37 to 39 was carried out in a manner similar to the method described for the synthesis of 33 above. Purification via flash chromatography on silica gel eluting with 3-20% MeOH in DCM yielded 39 in 77% yield (final step) as an oil. ¹H NMR (CDCl₃, 300 MHz) δ 9.12 (s, 1H), 8.80-8.77 (m, 2H), 8.17 (broad s, 1H), 8.03-7.86 (m, 3H), 7.52-7.43 (m, 3H), 7.24-7.21 (m, 1H), 3.98-3.93 (m, 2H), 2.79 (s, 3H), 2.66-2.62 (m, 2H), 2.47 (s, 3H), 2.40 (s, 6H), 2.01-1.93 (m, 2H). Mass spectrum (ESI⁺): m/z=385 [M+1].

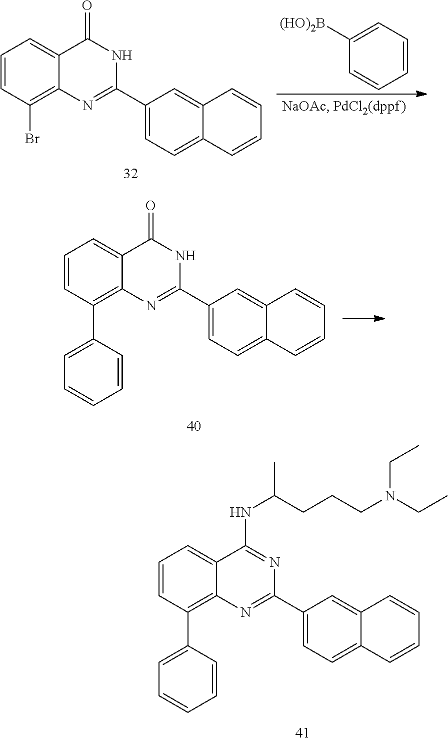 [SMILES: O=c1[nH]c(-c2ccc3ccccc3c2)nc2c(Br)cccc12 ; OB(O)c1ccccc1 ; O=c1[nH]c(-c2ccc3ccccc3c2)nc2c(-c3ccccc3)cccc12 ; CCN(CC)CCCC(C)Nc1nc(-c2ccc3ccccc3c2)nc2c(-c3ccccc3)cccc12]

A 20 mL screw-top vial with stir bar was charged with 32 (0.100 g, 0.28 mmol, 1 eq), phenylboronic acid (Combi-Blocks, 0.0521 g, 0.43 mmol, 1.5 eq), and NaOAc (0.105 g, 1.28 mmol, 4.5 eq). 1,4-dioxane/water (3:1, 7 mL) was added. The resulting stirred mixture was sparged with Ar (˜10 min). The vial was sealed and the reaction was heated to 95° C. overnight. After cooling to room temperature the reaction was diluted with water/hexanes, and the solids were collected via vacuum filtration. The filter cake was triturated with 20% DCM/hexanes and air dried. 0.083 g (0.24 mmol, 85% yield) of 40 was isolated as an ash colored solid. Mass spectrum (ESI⁺): m/z=349 [M+1].

Conversion of 40 to 41 was carried out in a manner similar to the method described for the synthesis of 11 above. Purification via flash chromatography on silica gel eluting with 3-20% MeOH in DCM yielded 41 in 63% yield as an oil. ¹H NMR (CDCl₃, 300 MHz) δ 9.02 (s, 1H), 8.61 (d, J=9 Hz, 1H), 7.97-7.77 (m, 7H), 7.58-7.43 (m, 6H), 6.12 (d, J=6.6 Hz, 1H), 4.81-4.77 (m, 1H), 2.64-2.57 (m, 6H), 1.88-1.74 (m, 4H), 1.46 (d, J=6.6 Hz, 3H), 1.06-1.01 (m, 6H). Mass spectrum (ESI⁺): m/z=489 [M+1].

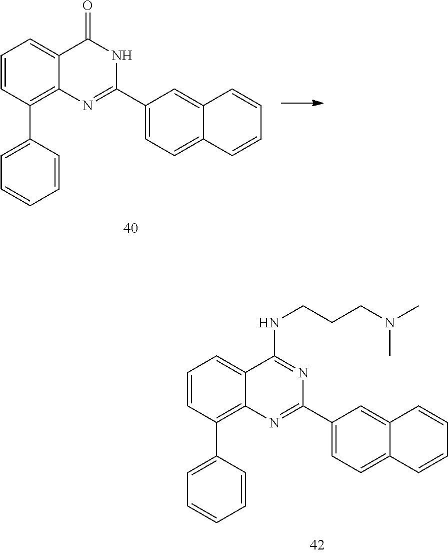 [SMILES: O=c1[nH]c(-c2ccc3ccccc3c2)nc2c(-c3ccccc3)cccc12 ; CN(C)CCCNc1nc(-c2ccc3ccccc3c2)nc2c(-c3ccccc3)cccc12]

Conversion of 40 to 42 was carried out in a manner similar to the method described for the synthesis of 11 above using 3-dimethylamino-1-propylamine. Purification via flash chromatography on silica gel eluting with 3-20% MeOH in DCM yielded 42 in 65% yield as a sticky solid. ¹H NMR (CDCl₃, 300 MHz) δ 9.04 (s, 1H), 8.63-8.60 (m, 1H), 8.53 (broad s, 1H), 7.97-7.79 (m, 6H), 7.71 (d, J=8.1 Hz, 1H), 7.58-7.42 (m, 6H), 4.02-3.97 (m, 2H), 2.73-2.69 (m, 2H), 2.46 (s, 6H), 2.04-2.00 (m, 2H). Mass spectrum (ESI⁺): m/z=433 [M+1].

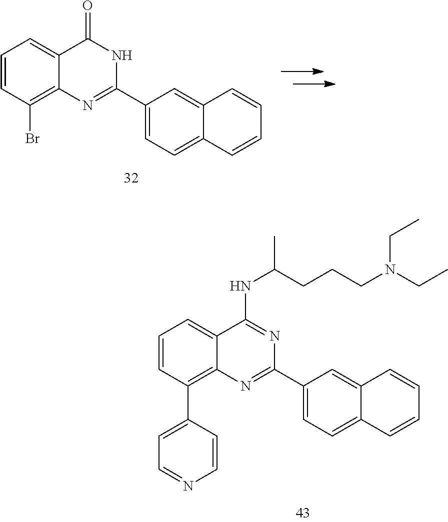 [SMILES: O=c1[nH]c(-c2ccc3ccccc3c2)nc2c(Br)cccc12 ; CCN(CC)CCCC(C)Nc1nc(-c2ccc3ccccc3c2)nc2c(-c3ccncc3)cccc12]

Conversion of 32 to 43 was carried out in a manner similar to the method described for the synthesis of 41 above using pyridine-4-boronic acid (Combi-Blocks). Purification via flash chromatography on silica gel eluting with 5-20% MeOH in DCM yielded 43 in 45% yield (final step) as an oil. ¹H NMR (CDCl₃, 300 MHz) δ 9.01 (s, 1H), 8.79 (d, J=4.8 Hz, 2H), 8.58 (d, J=8.4 Hz, 1H), 7.98-7.81 (m, 7H), 7.51-7.47 (m, 3H), 6.21 (d, J=6.6 Hz, 1H), 4.81-4.76 (m, 1H), 2.61-2.52 (m, 6H), 1.88-1.70 (m, 4H), 1.47 (d, J=6.6 Hz, 3H), 1.05-1.00 (m, 6H). Mass spectrum (ESI⁺): m/z=490 [M+1].

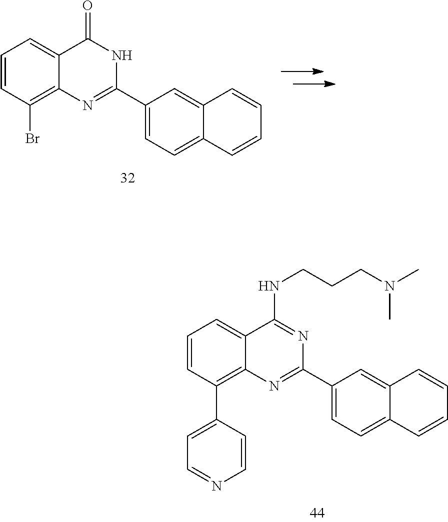 [SMILES: O=c1[nH]c(-c2ccc3ccccc3c2)nc2c(Br)cccc12 ; CN(C)CCCNc1nc(-c2ccc3ccccc3c2)nc2c(-c3ccncc3)cccc12]

Conversion of 32 to 44 was carried out in a manner similar to the method described for the synthesis of 41 above. Purification via flash chromatography on silica gel eluting with 5-20% MeOH in DCM yielded 44 in 49% yield (final step) as an oil. ¹H NMR (CDCl₃, 300 MHz) δ 9.03 (s, 1H), 8.78 (broad s, 3H), 8.60-8.57 (m, 1H), 7.98-7.95 (m, 1H), 7.90-7.79 (m, 5H), 7.73 (d, J=8.4 Hz, 1H), 7.51-7.46 (m, 3H), 4.03-3.97 (m, 2H), 2.71-2.70 (m, 2H), 2.44 (s, 6H), 2.04-1.97 (m, 2H). Mass spectrum (ESI⁺): m/z=434 [M+1].

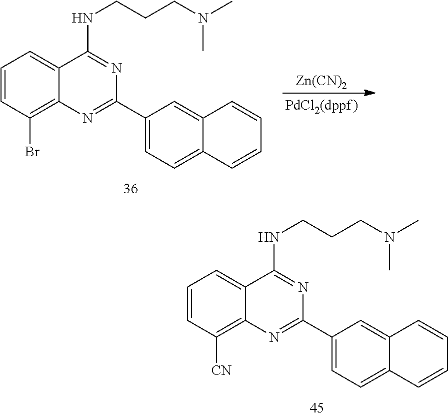

A 1 dram vial with stir bar was charged with 36 (0.017 g, 0.053 mmol, 1 eq). The vial was evacuated and back-filled with Ar (×3). Anh DMF (1 mL) was added. The vial was sealed with a septum, and the stirred solution was sparged with Ar (˜5 min). Zn(CN)₂ (0.0063 g, 0.053 mmol, 1 eq) and PdCl₂(dppf) (0.0079 g, 0.011 mmol, 0.2 eq) were added. The vial was closed (screw cap), and the reaction was heated to 110° C. overnight. After cooling to room temperature the reaction was poured into NH₄OH/ice with stirring. After 30 min the mixture was diluted with EtOAc and filtered through Celite. The layers were separated. The organic layer was washed with water (×2), brine (×1), and dried over Na₂SO₄. The solids were filtered off, and the volatiles were removed via rotary evaporation. Purification via flash chromatography on silica gel eluting with 5-20% MeOH in DCM followed by prep TLC eluting with 2:10:88 (NH₄OH (aq, sat)/MeOH/EtOAc yielded 0.0105 g (0.028 mmol, 52% yield) of 45 as a solid. ¹H NMR (CDCl₃, 300 MHz) δ 9.17 (s, 1H), 9.12 (broad s, 1H), 8.81-8.78 (m, 1H), 8.05-8.03 (m, 2H), 7.94-7.38 (m, 3H), 7.55-7.50 (m, 2H), 7.39-7.36 (m, 1H), 3.99-3.94 (m, 2H), 2.73-2.69 (m, 2H), 2.44 (s, 6H), 2.00-1.96 (m, 2H). Mass spectrum (ESI⁺): m/z=382 [M+1].

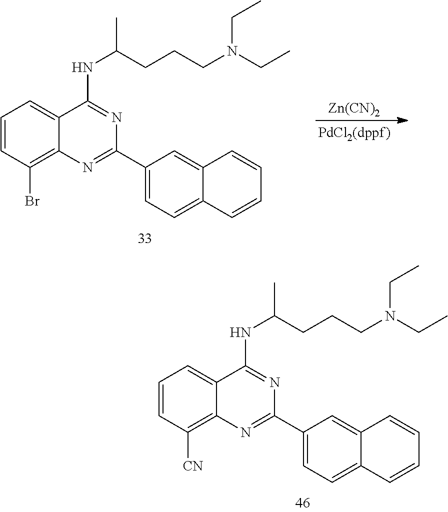

Conversion of 33 to 46 was carried out in a manner similar to the method described for the synthesis of 45 above. Purification via flash chromatography on silica gel eluting with 5-20% MeOH in DCM followed by prep TLC eluting with 2:10:88 (NH₄OH (aq, sat)/MeOH/EtOAc yielded 0.0054 g (0.0012 mmol, 26% yield) of 46 as an oil. ¹H NMR (CDCl₃, 300 MHz) δ 9.12 (s, 1H), 8.80-8.78 (m, 1H), 8.45-8.42 (m, 1H), 8.08-8.03 (m, 2H), 7.95-7.88 (m, 2H), 7.56-7.37 (m, 4H), 4.79 (broad s, 1H), 2.87-2.73 (m, 6H), 1.99-1.87 (m, 4H), 1.46 (d, J=6.6 Hz, 3H), 1.17-1.25 (m, 6H). Mass spectrum (ESI⁺): m/z=438 [M+1].

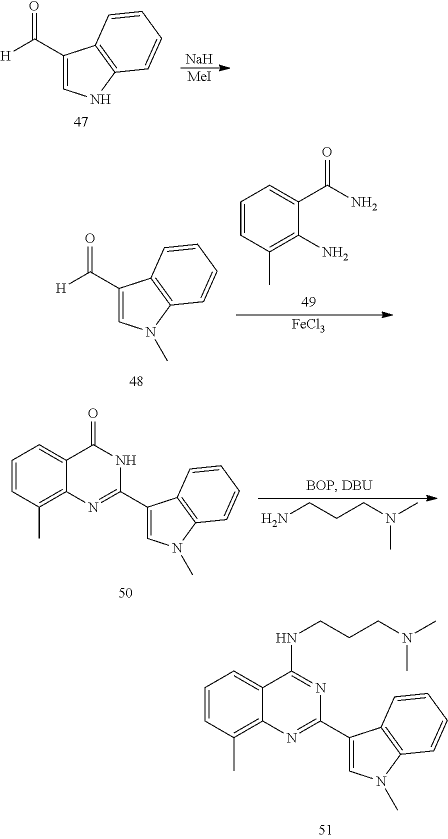

A 100 mL round bottom flask with stir bar was charged with 47 (Combi-Blocks, 0.500 g, 3.4 mmol, 1 eq). The flask was evacuated and back-filled with Ar (×3). Anh. DMF (10 mL) was added. The resulting solution was cooled to 0° C. with stirring. NaH (60% in oil, 0.179 g, 4.5 mmol, 1.3 eq) was added. After 45 min MeI (0.43 mL, 0.98 g, 6.9 mmol, 2 eq) was added, and the reaction was stirred at 0° C. to room temperature overnight. The reaction was diluted with water and extracted with EtOAc (×1). The layers were separated. The organic layer was washed with water (×2), brine (×1), and dried over Na₂SO₄. The solids were filtered off, and the volatiles were removed via rotary evaporation. The crude was plug filtered through silica gel eluting with 20% EtOAc/hexanes yielding 0.53 g (3.3 mmol, 98% yield) of 48 as a red oil which gradually solidified.

49 was prepared from 13 in 76% yield following the procedure for the synthesis of 31 above.

A 20 mL vial with stir bar was charged with 49 (0.0822 g, 0.55 mmol, 1 eq), 48 (0.0915 g, 0.57 mmol, 1.05 eq), and FeCl₃ (0.0444 g, 0.27 mmol, 0.5 eq). Water (1 mL) was added. The vial was closed (screw cap), and the mixture was heated to 110° C. overnight. After cooling to room temperature the mixture was diluted with water. The solids were collected via vacuum filtration. The filter cake was triturated with water followed by MeOH and air dried. 0.06 g (0.21 mmol, 38% yield) of crude 50 was isolated as a solid. Mass spectrum (ESI⁺): m/z=290 [M+1].

Conversion of 50 to 51 was carried according to the method described for the synthesis of 11 above using 3-dimethylamino-1-propylamine. Purification via flash chromatography on silica gel eluting with 5-20% MeOH in DCM yielded 51 in 25% yield as an oil. ¹H NMR (CDCl₃, 300 MHz) δ 8.92-8.89 (m, 1H), 8.10 (s, 1H), 7.98 (broad s, 1H), 7.53-7.50 (m, 2H), 7.53-7.17 (m, 4H), 3.87 (broad s, 5H), 2.81 (s, 3H), 2.66 (m, 2H), 2.41 (s, 6H), 2.01-1.93 (m, 2H). Mass spectrum (ESI⁺): m/z=374 [M+1].

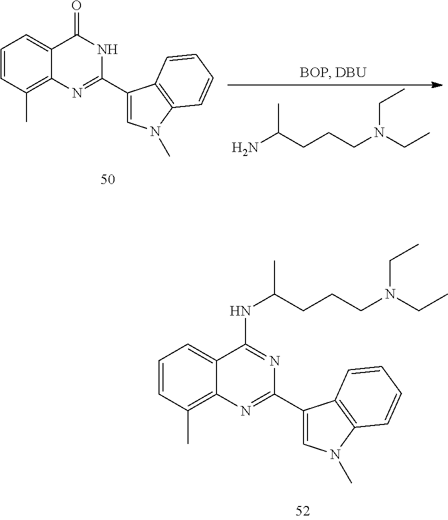 [SMILES: Cc1cccc2c(=O)[nH]c(-c3cn(C)c4ccccc34)nc12 ; CCN(CC)CCCC(C)N ; CCN(CC)CCCC(C)Nc1nc(-c2cn(C)c3ccccc23)nc2c(C)cccc12]

Conversion of 50 to 52 was carried according to the method described for the synthesis of 11 above. Purification via flash chromatography on silica gel eluting with 4-20% MeOH in DCM yielded 52 in 23% yield as an oil. ¹H NMR (CDCl₃, 300 MHz) δ 8.90-8.87 (m, 1H), 8.08 (s, 1H), 7.57-7.52 (m, 2H), 7.38-7.18 (m, 4H), 5.76 (d, J=6.9 Hz, 1H), 4.71-4.67 (m, 1H), 3.89 (s, 3H), 2.82 (s, 3H), 2.65-2.58 (m, 6H), 1.80-1.62 (m, 4H), 1.40 (d, J=6.3 Hz, 3H), 1.06-1.01 (m, 6H). Mass spectrum (ESI⁺): m/z=430 [M+1].

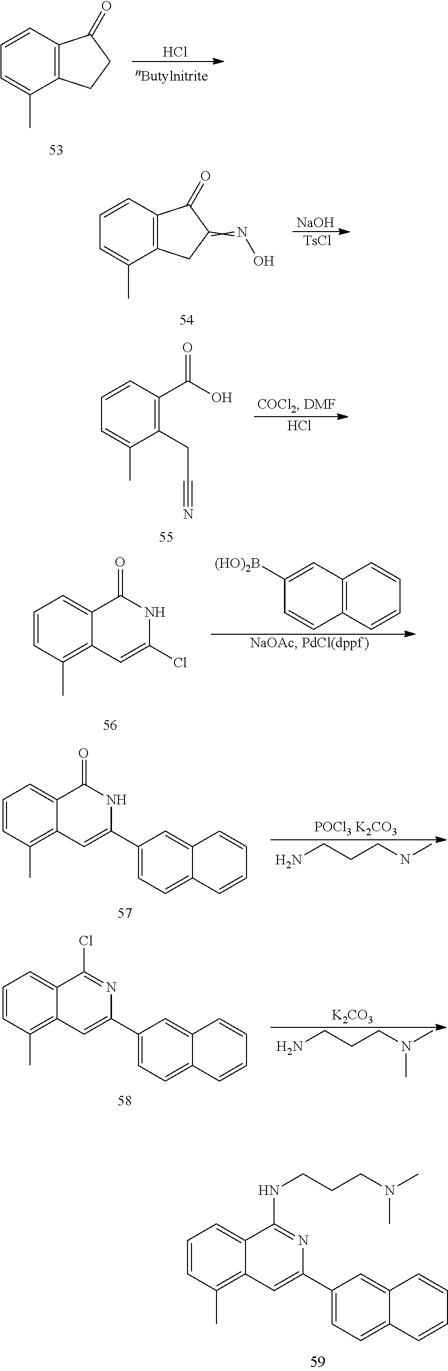

A 50 mL round bottom flask with stir bar charged with 53 (Aldrich, 0.500 g, 3.4 mmol, 1 eq). The system was evacuated and back-filled with Ar (×3). Anh. MeOH (13 mL) was added, and the resulting solution was treated with HCl (37%, 0.34 mL, 0.15 g, 4.4 mmol, 1.3 eq) with stirring. The reaction was heated to 40° C., and a solution of n-butylnitrite (0.44 mL, 0.39 g, 3.8 mmol, 1.1 eq) in an h. MeOH (3 mL) was added dropwise. After 5 h the reaction was cooled to room temperature, and the volatiles were removed via rotary evaporation. The residue was triturated with water, and the solids were collected via vacuum filtration. The filter cake was triturated with 10% MeOH/water and air dried. 0.442 g (2.5 mmol, 74% yield) of 54 was collected as a yellow solid.

A 50 mL round bottom flask with stir bar was charged with 54 (0.442 g, 2.5 mmol, 1 eq). NaOH (1 M, 10 mL, 10 mmol, 4 eq) was added, and the mixture was heated to 50° C. with stirring. TsCl (0.625 g, 3.3 mmol, 1.3 eq) was added, and the temperature was increased to 85° C. After 15 min the reaction was cooled to room temperature, and the mixture was filtered. The filtrate was adjusted to pH=2-3 with HCl (1 M). The solid which formed was collected via vacuum filtration. The filter cake was rinsed with water and air dried. 0.2875 g (1.6 mmol, 66% yield) of 55 was collected. Mass spectrum (ESI⁺): m/z=176 [M+1].

A 25 mL round bottom flask with stir bar was charged with 55 (0.167 g, 0.95 mmol, 1 eq). The flask was evacuated and back-filled with Ar (×3). Anh. DCM (5 mL) was added. The resulting stirred solution was treated with COCl₂ (0.32 mL, 0.48 g, 3.8 mmol, 4 eq) followed by DMF (2 drops). A homogeneous solution gradually formed. After 2 h the volatiles were removed via rotary evaporation. The residue was dissolved in toluene, and the volatiles were removed via rotary evaporation to yield the acid chloride. The flask was evacuated and back-filled with Ar (×3). HCl (4.0 M in 1,4-dioxane, 3 mL, 12 mmol, 12 eq) was added with stirring. A homogeneous solution gradually formed. After stirring overnight the volatiles were removed via rotary evaporation. The residue was diluted with water, and the solids were collected via vacuum filtration. The filter cake was triturated with 20% DCM/hexanes and air dried yielding 0.182 g (0.94 mmol, 99% yield) of 56 as an off-white solid. Mass spectrum (ESI⁺): m/z=194 [M+1].

Conversion of 56 to 57 was carried according to the method described for the synthesis of 40 above using naphthalene-2-boronic acid (Combi-Blocks). 57 was isolated in 98% yield as a charcoal colored solid. Mass spectrum (ESI⁺): m/z=286 [M+1].

A 25 mL round bottom flask with stir bar and reflux condenser was charged with 57 (0.03 g, 0.1 mmol, 1 eq). The system was evacuated and back-filled with Ar (×3). POCl₃ (1 mL) was added, and the reaction was heated to reflux. After 5 h the reaction was cooled to room temperature. The volatiles were removed via rotary evaporation. The residue was dissolve in toluene, and the volatiles were removed via rotary evaporation. The flask was evacuated and back-filled with Ar (×3). Anhydrous DMF (3 mL) was added. The resulting stirred solution was treated with K₂CO₃ (0.0291 g, 0.21 mmol, 2 eq) followed by 3-dimethylamino-1-propylamine (0.020 mL, 0.016 g, 0.016 mmol, 1.5 eq) 30 min later. The reaction was heated to 60° C. overnight. The reaction was cooled to room temperature and diluted with water. The resulting mixture was filtered through Celite, rinsing with water. The flask and filter cake were rinsed with EtOAc (×3). The combined EtOAc fractions were washed with brine (×1) and dried over Na₂SO₄. The solids were filtered off, and the volatiles were removed via rotary evaporation. The crude was purified by plug filtration through silica gel eluting with DCM. 0.022 g (0.07 mmol, 72% yield) of 58 was isolated. Mass spectrum (ESI⁺): m/z=304 [M+1].

A 20 mL vial with stir bar was charged with 58 (0.022 g, 0.072 mmol, 1 eq) and K₂CO₃ (0.02 g, 0.14 mmol, 2 eq). The vial was evacuated and back-filled with Ar (×3). Anhydrous DMF (1 mL) was added followed by 3-dimethylamino-1-propylamine (0.018 mL, 0.015 g, 0.14 mmol, 2 eq). The vial was sealed (screw cap), and the mixture was heated to 100° C. overnight with stirring. The reaction was cooled to room temperature, diluted with EtOAc/water, and filtered through Celite. The layers were separated. The organic layer was washed with water (×2), brine (×1), and dried over Na₂SO₄. The solids were filtered off, and the volatiles were removed via rotary evaporation. Purification via flash chromatography on silica gel yielded the crude product. The crude was plug filtered through basic alumina eluting with 1:1 EtOAc/hexanes followed by 10% MeOH in EtOAc yielded 0.0029 g (0.008 mmol, 11% yield) of 59. ¹H NMR (CDCl₃, 300 MHz) δ 8.66 (d, J=8.7 Hz, 1H), 7.98-7.85 (m, 3H), 7.67 (d, J=8.1 Hz, 1H), 7.60 (s, 1H), 7.49-7.30 (m, 4H), 3.94-3.90 (m, 1H), 2.70 (s, 3H), 2.67-2.63 (m, 2H), 2.41 (s, 6H), 2.04-2.00 (m, 2H). Mass spectrum (ESI⁺): m/z=370 [M+1].

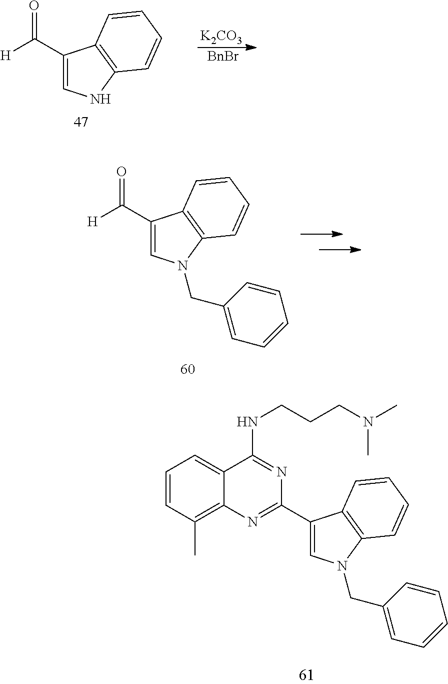

A 50 mL round bottom flask with stir bar was charged with 47 (0.40 g, 2.75 mmol, 1 eq). The flask was evacuated and back-filled with Ar (×3). Anh. DMF (5 mL) was added. The resulting solution was treated sequentially with BnBr (0.36 mL, 0.52 g, 3.03 mmol, 1.1 eq) and K₂CO₃ (0.4379 g, 3.17 mmol, 1.15 eq). The reaction was stirred overnight. The reaction was diluted with 1:1 EtOAc/hexanes and filtered through Celite. The filtrate was washed with water (×3), brine (×1), and dried over Na₂SO₄. The solids were filtered off, and the volatiles were removed via rotary evaporation.

Conversion of 60 to 61 was carried according to the method described for the synthesis of 51 above. Purification via flash chromatography on silica gel eluting with 0-20% MeOH in DCM yielded 61 in 9% yield (final step) as an oil. ¹H NMR (CDCl₃, 300 MHz) δ 8.96-8.93 (m, 1H), 8.21-8.20 (m, 1H), 8.01 (broad s, 1H), 7.54 (broad s, 2H), 7.28-7.20 (m, 10H), 5.44 (s, 2H), 3.89 (broad s, 2H), 2.82 (s, 3H), 2.70 (broad s, 2H), 2.45 (s, 6H), 2.00 (broad s, 2H). Mass spectrum (ESI⁺): m/z=450 [M+1].

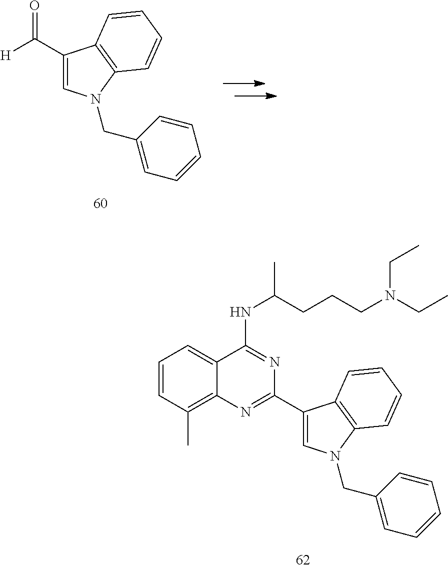 [SMILES: O=Cc1cn(Cc2ccccc2)c2ccccc12 ; CCN(CC)CCCC(C)Nc1nc(-c2cn(Cc3ccccc3)c3ccccc23)nc2c(C)cccc12]

Conversion of 60 to 62 was carried according to the method described for the synthesis of 51 above. Purification via flash chromatography on silica gel eluting with 0-20% MeOH in DCM yielded 62 in 9% yield (final step) as an oil. ¹H NMR (CDCl₃, 300 MHz) δ 8.93 (d, J=7.8 Hz, 1H), 8.19 (s, 1H), 7.57-7.53 (m, 2H), 7.32-7.16 (m, 9H), 5.76 (d, J=7.8 Hz, 1H), 5.44 (s, 2H), 4.69-4.65 (m, 1H), 2.82 (s, 3H), 2.62-2.55 (m, 6H), 1.80-1.70 (m, 4H), 1.39 (d, J=6.6 Hz, 3H), 1.03-0.98 (m, 6H). Mass spectrum (ESI⁺): m/z=506 [M+1].

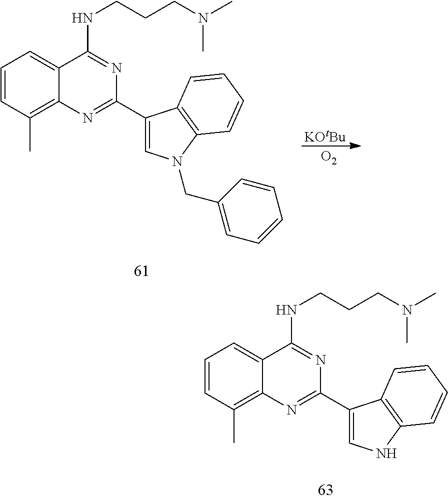 [SMILES: Cc1cccc2c(NCCCN(C)C)nc(-c3cn(Cc4ccccc4)c4ccccc34)nc12 ; "O2" ; Cc1cccc2c(NCCCN(C)C)nc(-c3c[nH]c4ccccc34)nc12 ; "KOtBu"]

A 25 mL round bottom flask with stir bar was charged with 61 (8.4 mg, 0.019 mmol, 1 eq). The flask was evacuated and back-filled with Ar (×3). Anh. DMSO (1 mL) was added followed by KO^{t}Bu (0.5 M in THF, 0.38 mL, 0.19 mmol, 10 eq). The resulting stirred solution was sparged with O₂ (15 min). The reaction was stirred under O₂ for 5 h. The reaction was quenched with water and diluted with EtOAc/NaHCO₃ (aq, sat). The layers were separated. The organic layer was washed with brine (×1) and dried over Na₂SO₄. The solids were filtered off, and the volatiles were removed via rotary evaporation. Purification by prep TLC eluting with 14% MeOH in DCM followed by prep TLC eluting with 10:2:88 (MeOH/NH₃ (aq, sat)/EtOAc) yielded 1.8 mg (26% yield) of 63 as an off-white solid. ¹H NMR (CDCl₃, 300 MHz) δ 8.91 (d, J=6.3 Hz, 1H), 8.45 (broad s, 1H), 8.23 (s, 1H), 7.80 (broad s, 1H), 7.66-7.64 (m, 1H), 7.54 (d, J=7.2 Hz, 1H), 7.42 (d, J=7.2 Hz, 1H), 7.32-7.21 (m, 3H), 3.92-3.88 (m, 2H), 2.82 (s, 3H), 2.77 (m, 2H), 2.48 (s, 6H), 2.00 (m, 2H). Mass spectrum (ESI⁺): m/z=360 [M+1].

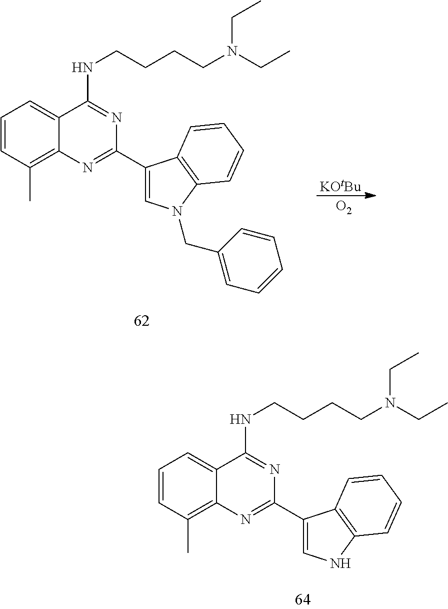 [SMILES: CCN(CC)CCCCNc1nc(-c2cn(Cc3ccccc3)c3ccccc23)nc2c(C)cccc12 ; "O2" ; CCN(CC)CCCCNc1nc(-c2c[nH]c3ccccc23)nc2c(C)cccc12 ; "KOtBu"]

Conversion of 62 to 64 was carried according to the method described for the synthesis of 63 above. 64 was isolated in 30% yield as an off-white solid. ¹H NMR (CDCl₃, 300 MHz) δ 8.90 (d, J=8.1 Hz, 1H), 8.76 (s, 1H), 8.22 (s, 1H), 7.60-7.53 (m, 2H), 7.42 (d, J=7.2 Hz, 1H), 7.32-7.20 (m, 5H), 5.78 (d, J=5.7 Hz, 1H), 4.66 (m, 1H), 2.83 (s, 3H), 2.71-2.61 (m, 6H), 1.87-1.62 (m, 4H), 1.39 (d, J=6.3 Hz, 3H), 1.07-1.02 (m, 6H). Mass spectrum (ESI⁺): m/z=416 [M+1].

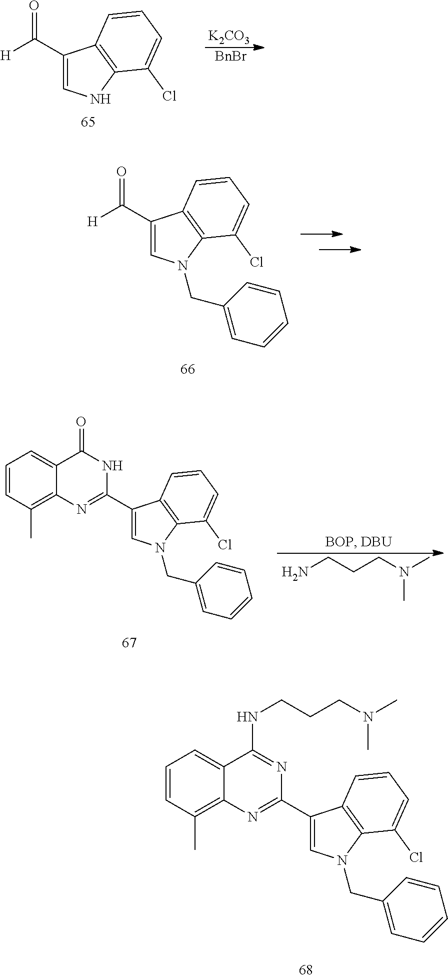

Conversion of 65 (Matrix Scientific) to 66 was carried according to the method described for the synthesis of 60 above. 66 was isolated in 93% yield as peach colored solid.

A 2.5 mL microwave vial with stir bar was charged with 49 (0.0797 g, 0.53 mmol, 1 eq), 66 (0.1432 g, 0.53 mmol, 1 eq), and InCl₃ (0.1174 g, 0.53 mmol, 1 eq). The vial was sealed with a septum. The vial was evacuated and back-filled with Ar (×3). Anh. DMF (2 mL) was added, and the reaction was stirred at room temperature over the weekend. LCMS indicated the reaction was not complete. The vial was capped, and the reaction was irradiated at 150° C. for 1 h. After cooling to room temperature the reaction was diluted with water, and the solids were collected via vacuum filtration. LCMS indicated the reaction was still not complete. The solids were dissolved in DMSO (2 mL), and the reaction was heated to 150° C. overnight. After cooling to room temperature the reaction was diluted with water. The resulting solids were collected via vacuum filtration, rinsing with water. The filter cake was triturated with MeOH and air dried yielding 0.089 g of crude 67. The crude was used in the next step without further purification. Mass spectrum (ESI⁺): m/z=400 [M+1].

Conversion of 67 to 68 was carried according to the method described for the synthesis of 11 above using 3-dimethylamino-1-propylamine. Purification via flash chromatography on silica gel eluting with 0-20% MeOH in DCM yielded 68 in 23% yield as a pale yellow solid. ¹H NMR (CDCl₃, 300 MHz) δ 8.98-8.95 (m, 1H), 8.21 (broad s, 1H), 8.16 (s, 1H), 7.53 (d, J=6.9 Hz, 1H), 7.44 (d, J=8.4 Hz, 1H), 7.30-7.18 (m, 6H), 7.08 (d, J=6.9 Hz, 2H), 5.87 (s, 2H), 3.87-3.82 (m, 2H), 2.80 (s, 3H), 2.62-2.58 (m, 2H), 2.37 (s, 6H), 1.94-1.86 (m, 2H). Mass spectrum (ESI⁺): m/z=484 [M+1].

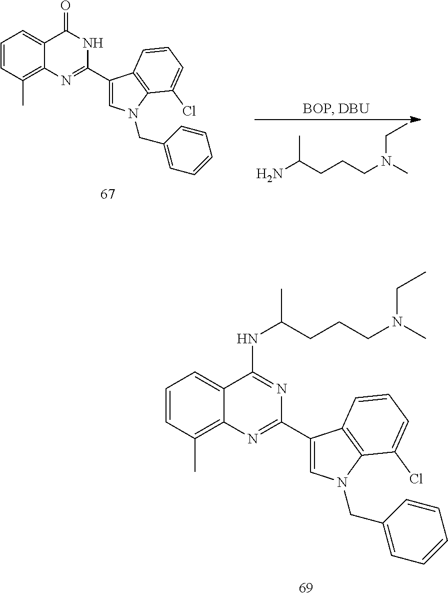

Conversion of 67 to 69 was carried according to the method described for the synthesis of 11 above. Purification via flash chromatography on silica gel eluting with 0-20% MeOH in DCM yielded 69 in 19% yield as a yellow oil. ¹H NMR (CDCl₃, 300 MHz) δ 8.97-8.94 (m, 1H), 8.14 (s, 1H), 7.55-7.49 (m, 2H), 7.31-7.18 (m, 6H), 7.08 (d, J=6.9 Hz, 2H), 5.87 (s, 2H), 5.72 (d, J=7.5 Hz, 1H), 4.63-4.58 (m, 1H), 2.81 (s, 3H), 2.54-2.46 (m, 6H), 1.77-1.61 (m, 4H), 1.37 (d, J=6.3 Hz, 3H), 1.00-0.95 (m, 6H). Mass spectrum (ESI⁺): m/z=540 [M+1].

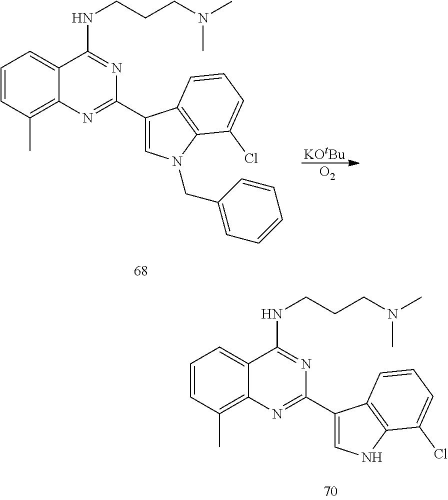

Conversion of 68 to 70 was carried according to the method described for the synthesis of 63 above. 70 was isolated in 32% yield as an off-white solid. ¹H NMR (CDCl₃, 300 MHz) δ 8.82 (d, J=6.9 Hz, 1H), 8.58 (broad s, 1H), 8.28 (s, 1H), 8.070 (broad s, 1H), 7.55-7.53 (m, 2H), 7.25-7.20 (m, 3H), 3.88 (m, 2H), 2.81 (s, 3H), 2.71-2.67 (m, 2H), 2.43 (s, 6H), 2.02-1.96 (m, 2H). Mass spectrum (ESI⁺): m/z=394 [M+1].

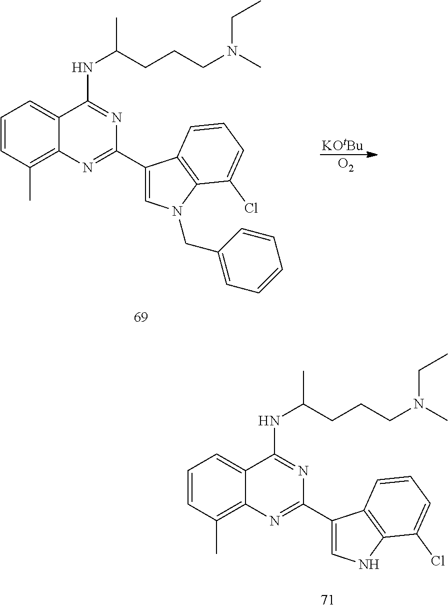 [SMILES: CCN(C)CCCC(C)Nc1nc(-c2cn(Cc3ccccc3)c3c(Cl)cccc23)nc2c(C)cccc12 ; "O2" ; CCN(C)CCCC(C)Nc1nc(-c2c[nH]c3c(Cl)cccc23)nc2c(C)cccc12 ; "KOtBu"]

Conversion of 69 to 71 was carried according to the method described for the synthesis of 63 above. 71 was isolated in 35% yield as an off-white solid. ¹H NMR (CDCl₃, 300 MHz) δ 8.93 (broad s, 1H), 8.82-8.79 (m, 1H), 8.28 (d, J=2.4 Hz, 1H), 7.63 (d, J=7.8 Hz, 1H), 7.55 (d, J=7.2 Hz, 1H), 7.25-7.20 (m, 3H), 5.91 (broad s, 1H), 4.65 (m, 1H), 2.82 (s, 3H), 2.75-2.68 (m, 6H), 1.89-1.62 (m, 4H), 1.40 (d, J=6.6 Hz, 3H), 1.10-1.05 (m, 6H). Mass spectrum (ESI⁺): m/z=450 [M+1].

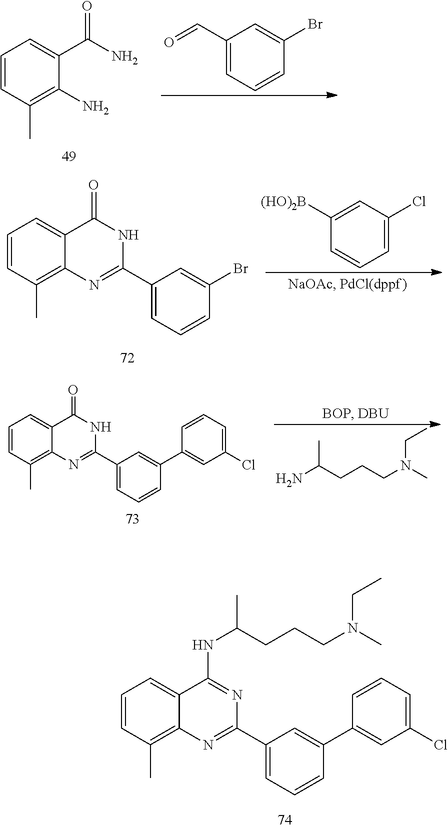

A 25 mL pear-shaped flask with stir bar was charged with 49 (0.30 g, 2.0 mmol, 1 eq) and 3-bromobenzaldehyde (Aldrich, 0.37 g, 2.0 mmol, 1 eq). DMSO (2 mL) was added followed by a couple of drops of water. The resulting stirred solution, open to the air, was heated to 100° C. overnight. LCMS indicated the reaction was not complete. The temperature was increased to 140° C. After 3 h the reaction was cooled to room temperature and diluted with water. The solids were collected via vacuum filtration. The filter cake was rinsed with water followed by 20% DCM/hexanes and air dried. 0.523 g (1.7 mmol, 83% yield) of 72 was collected as an off-white solid. Mass spectrum (ESI⁺): m/z=315 [M+1].

Conversion of 72 to 73 was carried according to the method described for the synthesis of 40 above using 3-chlorobenzeneboronic acid (Combi-Blocks). 73 was isolated in 100% yield as a charcoal colored solid. Mass spectrum (ESI⁺): m/z=347 [M+1].

Conversion of 73 to 74 was carried according to the method described for the synthesis of 11 above. Purification via flash chromatography on silica gel eluting with 0-100% solvent B (Solvent A=EtOAc; Solvent B=10:1:89 MeOH/NH₃ (aq, sat)/EtOAc yielded 74 in 24% yield as a sticky amber semi-solid. ¹H NMR (CDCl₃, 300 MHz) δ 8.82 (s, 1H), 8.62 (d, J=7.8 Hz, 1H), 7.70-7.53 (m, 6H), 7.44-7.27 (m, 4H), 5.90 (d, J=7.5 Hz, 1H), 4.70-4.63 (m, 1H), 2.80 (s, 3H), 2.57-2.48 (m, 6H), 2.24 (s, 3H), 1.83-1.64 (m, 4H), 1.40 (d, J=6.3 Hz, 3H), 1.01-0.97 (m, 6H). Mass spectrum (ESI⁺): m/z=487 [M+1].

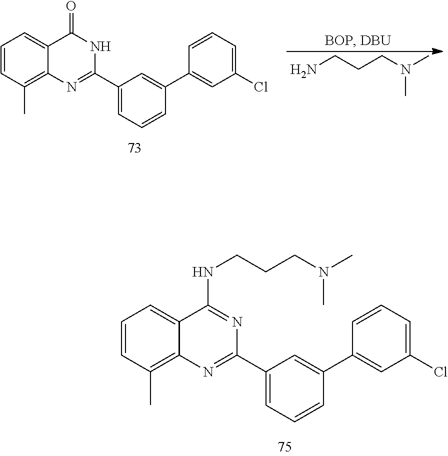

Conversion of 73 to 75 was carried according to the method described for the synthesis of 11 above using 3-dimethylamino-1-propylamine. Purification via flash chromatography on silica gel eluting with 0-100% solvent B (Solvent A=EtOAc; Solvent B=10:1:89 MeOH/NH₃ (aq, sat)/EtOAc yielded 75 in 62% yield as a sticky amber semi-solid. ¹H NMR (CDCl₃, 300 MHz) δ 8.83 (s, 1H), 8.64 (d, J=7.5 Hz, 1H), 8.40 (s, 1H), 7.71-7.28 (m, 9H), 3.90-3.89 (m, 2H), 2.80 (s, 3H), 2.63-2.60 (m, 2H), 2.37 (s, 6H), 1.96-1.87 (m, 2H). Mass spectrum (ESI⁺): m/z=431 [M+1].

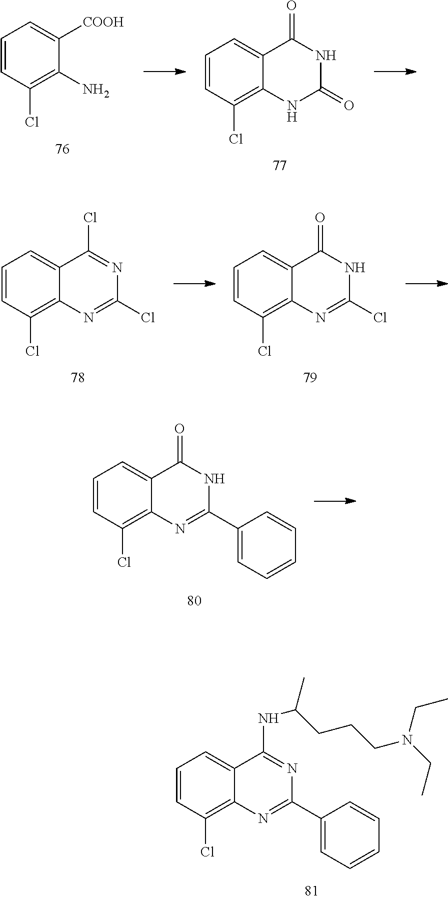 [SMILES: Nc1c(Cl)cccc1C(=O)O ; O=c1[nH]c(=O)c2cccc(Cl)c2[nH]1 ; Clc1nc(Cl)c2cccc(Cl)c2n1 ; O=c1[nH]c(Cl)nc2c(Cl)cccc12 ; O=c1[nH]c(-c2ccccc2)nc2c(Cl)cccc12 ; CCN(CC)CCCC(C)Nc1nc(-c2ccccc2)nc2c(Cl)cccc12]

2-Amino-3-chlorobenzoic acid 76 (5.0 g, 29.24 mmol) and urea (3.5 g, 58.27 mmol) were heated together at 140° C. for 2 hours and at 180° C. for 24 h. Then reaction mixture was cooled and triturated with hot water followed by ethyl acetate. The solid was filtered and dried to give 77 (3.8 g, 67%). Mass spectrum (ESI⁺): m/z=197 [M+1].

To the compound 77 (3.8 g, 19.33 mmol), 60 mL POCl₃ was added. The mixture was then heated at reflux for 16 hours. Excess POCl₃ was removed in vacuum, and the residue was purified by column chromatography to provide 78 (3.11 g, 76%). Mass spectrum (ESI⁺): m/z=233 [M+1].

A mixture of 45 mL of 1N NaOH, 100 mL of THF, and 3.11 g of 78 was stirred at room temperature under N₂ for 2 h. The solution was acidified with to PH 5. Then reaction mixture was diluted with water and ethyl acetate. Organic layer was separated, dried and evaporated. 79 was carried to the next step in the form of crude residue. Mass spectrum (ESI⁺): m/z=215 [M+1].

To the chloro compound 79 (75 mg, 0.35 mmol), phenyl boronic acid (49 mg, 0.40 mmol), sodium acetate (136 mg, 1.66 mmol) in ethanol/toluene/water (2/5/2 ml), PdCl₂(dppf) (0.07 mmol) was added and heated to 80° C. for 16 h. Then reaction mixture was cooled and diluted with ethyl acetate. Layers were separated and the organic layer was washed with water, brine and dried. Crude residue was subjected to column chromatography to yield 80 (30 mg, 30%). Mass spectrum (ESI⁺): m/z=257 [M+1].

To the suspension of quinazolinone 80 (30 mg, 0.12 mmol) in acetonitrile, BOP (69 mg, 0.16 mmol) was added followed by DBU (27 mg, 0.18 mmol) and stirred for 20 min at room temperature. Then 2-amino-5-diethylaminopentane (24 mg, 0.18 mmol) was added and stirred for 15 min at room temperature and 1.5 h at 60° C. Then the reaction mixture was cooled, diluted with ethyl acetate, washed with water, brine and dried. Crude residue was column chromatographed to yield 81 in 28% yield.

¹H NMR (CDCl₃): δ 8.60-8.57 (m, 2H), 7.96-7.81 (m, 2H), 7.50-7.31 (m, 4H), 3.01-2.83 (m, 7H), 1.80-1.76 (m, 4H), 1.42-1.39 (d, 3H, J=6 Hz), 1.17-1.12 (t, 6H, J=6.6 Hz).

Mass spectrum (ESI⁺): m/z=397 [M+1].

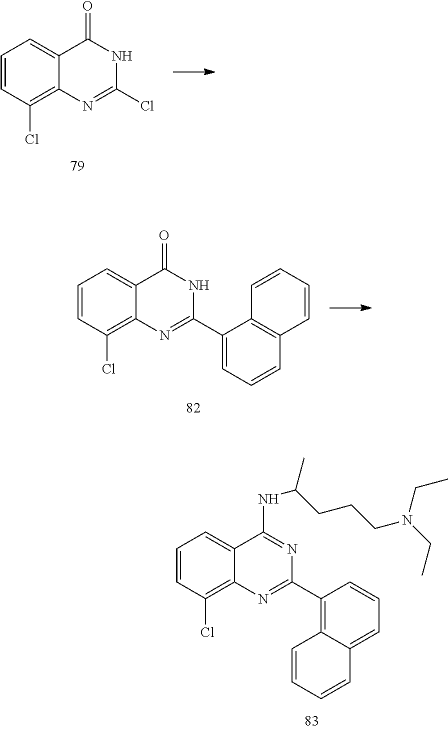 [SMILES: O=c1[nH]c(Cl)nc2c(Cl)cccc12 ; O=c1[nH]c(-c2cccc3ccccc23)nc2c(Cl)cccc12 ; CCN(CC)CCCC(C)Nc1nc(-c2cccc3ccccc23)nc2c(Cl)cccc12]

To the chloro compound 79 (90 mg, 0.42 mmol), naphthyl boronic acid (108 mg, 0.63 mmol), sodium acetate (163 mg, 1.99 mmol) in ethanol/toluene/water (2/5/2 ml), PdCl₂(dppf) (62 mg, 0.08 mmol) was added and heated to 80° C. for 16 h. Then reaction mixture was cooled and diluted with ethyl acetate. Layers were separated and the organic layer was washed with water, brine and dried. Crude residue was subjected to column chromatography to yield 82 in 50% yield. Mass spectrum (ESI⁺): m/z=307 [M+1].

To the suspension of quinazolinone 7 (70 mg, 0.23 mmol) in acetonitrile, BOP (131 mg, 0.30 mmol) was added followed by DBU (52 mg, 0.34 mmol) and stirred for 20 min at room temperature. Then 2-amino-5-diethylaminopentane (46 mg, 0.34 mmol) was added and stirred for 15 min at room temperature and 2.5 h at 60° C. Then the reaction mixture was cooled, diluted with ethyl acetate, washed with water, brine and dried. Crude residue was column chromatographed to yield 83 in 34% yield.

¹H NMR (CDCl₃): δ 9.12 (s, 1H), 8.77-8.74 (d, 1H, J=6 Hz), 8.04-7.80 (m, 4H), 7.52-7.49 (m, 2H), 7.34-7.29 (t, 1H, J=7.8 Hz), 6.72 (br s, 1H), 2.79-2.62 (m, 7H), 2.05-1.81 (m, 4H), 1.46-1.44 (d, 3H, J=6 Hz), 1.13-1.08 (t, 6H, J=7.2 Hz).

Mass spectrum (ESI⁺): m/z=447 [M+1].

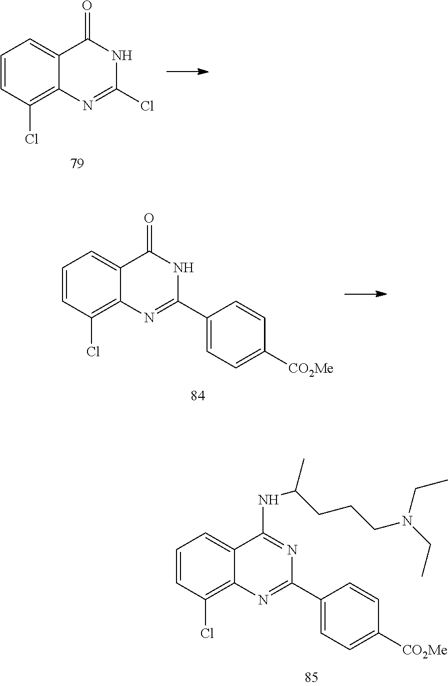 [SMILES: O=c1[nH]c(Cl)nc2c(Cl)cccc12 ; COC(=O)c1ccc(-c2nc3c(Cl)cccc3c(=O)[nH]2)cc1 ; CCN(CC)CCCC(C)Nc1nc(-c2ccc(C(=O)OC)cc2)nc2c(Cl)cccc12]

To the chloro compound 79 (90 mg, 0.42 mmol), 4-methoxycarbonylphenylboronic acid (113 mg, 0.63 mmol), sodium acetate (163 mg, 1.99 mmol) in ethanol/toluene/water (2/5/2 ml), PdCl₂(dppf) (62 mg, 0.08 mmol) was added and heated to 80° C. for 16 h. Then reaction mixture was cooled and diluted with ethyl acetate. Layers were separated and the organic layer was washed with water, brine and dried. Crude residue was subjected to column chromatography to yield 84 in 65% yield.

Mass spectrum (ESI⁺): m/z=315 [M+1].

To the suspension of quinazolinone 9 (63 mg, 0.20 mmol) in acetonitrile, BOP (115 mg, 0.26 mmol) was added followed by DBU (46 mg, 0.30 mmol) and stirred for 20 min at room temperature. Then 2-amino-5-diethylaminopentane (46 mg, 0.34 mmol) was added and stirred for 15 min at room temperature and 2.5 h at 60° C. Then the reaction mixture was cooled, diluted with ethyl acetate, washed with water, brine and dried. Crude residue was column chromatographed to yield 85 in 34% yield.

¹H NMR (CDCl₃): δ 9.12 (s, 1H), 8.69-8.66 (d, 2H, J=6 Hz), 8.15-8.12 (d, 2H, J=6 Hz), 7.88-7.8 (m, 3H), 7.37-7.32 (t, 1H, J=7.8 Hz), 6.35 (br s, 1H), 3.95 (s, 3H), 2.85-2.78 (m, 7H), 1.92-1.76 (m, 4H), 1.40-1.38 (d, 3H, J=6 Hz), 1.14-1.09 (t, 6H, J=7.2 Hz).

Mass spectrum (ESI⁺): m/z=455 [M+1].

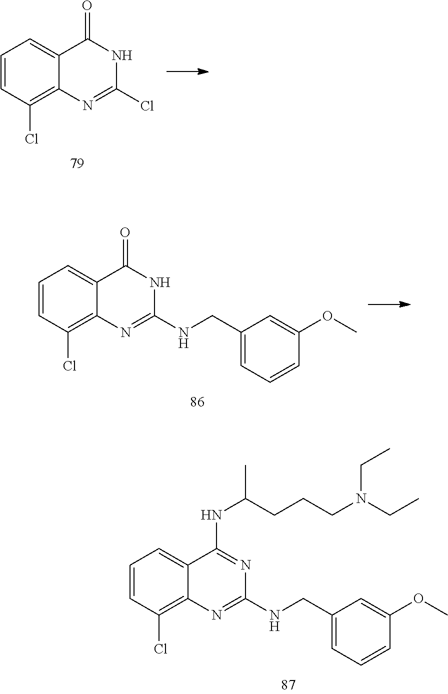 [SMILES: O=c1[nH]c(Cl)nc2c(Cl)cccc12 ; COc1cccc(CNc2nc3c(Cl)cccc3c(=O)[nH]2)c1 ; CCN(CC)CCCC(C)Nc1nc(NCc2cccc(OC)c2)nc2c(Cl)cccc12]

To the 2,8-dichloroquinazolin-4(3H) one 79 (60 mg, 0.28 mmol) in i-PrOH (3 ml), DIPEA (72 mg, 0.56 mmol) was added followed by 3-methoxy benzylamine (96 mg, 0.70 mmol). Reaction mixture was heated to 80° C. for 16 h. Then reaction mixture was cooled and volatiles removed in vacuum. Crude residue was subjected to column chromatography to yield 86 in 86% yield.

Mass spectrum (ESI⁺): m/z=316 [M+1].

To the suspension of quinazolinone 86 (75 mg, 0.23 mmol) in acetonitrile, BOP (137 mg, 0.31 mmol) was added followed by DBU (27 mg, 0.18 mmol) and stirred for 20 min at room temperature. Then 2-amino-5-diethylaminopentane (52 mg, 0.34 mmol) was added and stirred for 15 min at room temperature and 1.5 h at 60° C. Then the reaction mixture was cooled, diluted with ethyl acetate, washed with water, brine and dried. Crude residue was column chromatographed to yield 87 in 55% yield.

¹H NMR (CDCl₃):7.72-7.69 (d, 2H, J=6 Hz), 7.62-7.59 (d, 2H, J=6 Hz), 8.15-8.12 (d, 2H, J=6 Hz), 7.28-7.16 (m, 3H), 6.77 (br s, 1H), 4.67 (s, 2H), 3.77 (s, 3H), 2.75-2.60 (m, 7H), 1.78-1.52 (m, 4H), 1.24-1.22 (d, 3H, J=6 Hz), 1.12-1.07 (t, 6H, J=7.2 Hz).

Mass spectrum (ESI⁺): m/z=456 [M+1].

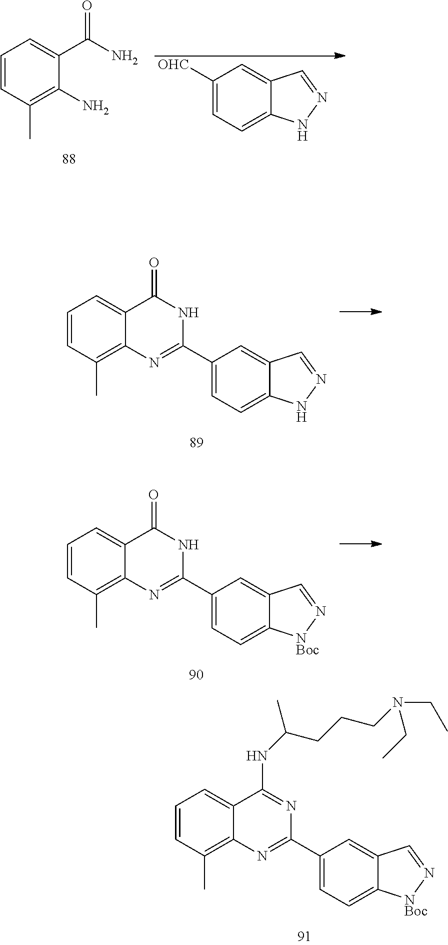 [SMILES: Cc1cccc(C(N)=O)c1N ; O=Cc1ccc2[nH]ncc2c1 ; Cc1cccc2c(=O)[nH]c(-c3ccc4[nH]ncc4c3)nc12 ; Cc1cccc2c(=O)[nH]c(-c3ccc4c(cnn4C(=O)OC(C)(C)C)c3)nc12 ; CCN(CC)CCCC(C)Nc1nc(-c2ccc3c(cnn3C(=O)OC(C)(C)C)c2)nc2c(C)cccc12]

To the amine 88 (150 mg, 2 mmol), 1H-indazole-4-carbaldehyde (145 mg, 1 mmol), iron(iii) chloride (65 mg, 0.4 mmol) was added followed by water (5 ml). Reaction mixture was then heated to 100° C. for 24 h. Then reaction mixture was cooled, filtered and the crude was carried to the next step.

Mass spectrum (ESI⁺): m/z=277 [M+1].

To the quinazolinone 89 (250 mg, 0.97 mmol) in DMF (5 ml), DMAP (23 mg, 0.19 mmol) and (Boc)₂O (316 mg, 1.45 mmol) were added and stirred at rt for 3 h. Then the reaction mixture was diluted with water, extracted with ethyl acetate. Organic layer was washed with water, brine and dried. Crude residue was column chromatographed to yield 53% of 90.

Mass spectrum (ESI⁺): m/z=377 [M+1].

To the solution of quinazolinone 90 (70 mg, 0.19 mmol) in DMF (5 ml), BOP (107 mg, 0.24 mmol) was added followed by DBU (43 mg, 0.48 mmol) and stirred for 20 min at room temperature. Then 2-amino-5-diethylaminopentane (76 mg, 0.28 mmol) was added and stirred for 15 min at room temperature and 6 h at 60° C. Then the reaction mixture was cooled, diluted with ethyl acetate, washed with water, brine and dried. Crude residue was column chromatographed to yield 91 in 20% yield.

¹H NMR (CDCl₃): 8.84 (s, 1H), 8.66-8.63 (d, 1H, J=6 Hz), 8.21-8.18 (d, 1H, J=6 Hz), 7.64-7.55 (m, 3H), 7.29-7.26 (t, 2H, J=6 Hz), 6.70-6.69 (d, 1H, J=3.6 Hz), 5.84 (br s, 1H), 4.76-4.72 (m, 1H), 2.81 (s, 3H), 2.60-2.53 (m, 7H), 1.78-1.70 (m, 12H), 1.42-1.40 (d, 3H, J=6 Hz), 1.04-0.99 (t, 6H, J=7.2 Hz).

Mass spectrum (ESI⁺): m/z=517 [M+1].

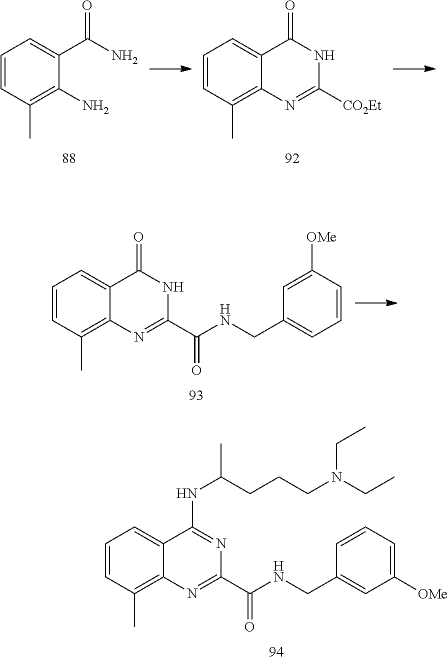 [SMILES: Cc1cccc(C(N)=O)c1N ; CCOC(=O)c1nc2c(C)cccc2c(=O)[nH]1 ; COc1cccc(CNC(=O)c2nc3c(C)cccc3c(=O)[nH]2)c1 ; CCN(CC)CCCC(C)Nc1nc(C(=O)NCc2cccc(OC)c2)nc2c(C)cccc12]

To the compound 88, diethyl oxalate was added and heated at 180-185° C. for 24 h. Then reaction mixture was cooled and filtered. Resulting crude was carried to the next step without any further purification.

To the ester 92 (80 mg, 0.34 mmol) in EtOH (3 ml), DIPEA (88 mg, 0.68 mmol) was added followed by 3-methoxy benzylamine (71 mg, 0.52 mmol). Reaction mixture was heated to 80° C. for 16 h. Then reaction mixture was cooled and volatiles removed in vacuum. Crude residue was subjected to column chromatography to yield 11 in 50% yield.

Mass spectrum (ESI⁺): m/z=324 [M+1].

To the solution of quinazolinone 93 (55 mg, 0.17 mmol) in DMF (3 ml), BOP (98 mg, 0.22 mmol) was added followed by DBU (38 mg, 0.25 mmol) and stirred for 20 min at room temperature. Then 2-amino-5-diethylaminopentane (40 mg, 0.25 mmol) was added and stirred for 15 min at room temperature and 1.5 h at 60° C. Then the reaction mixture was cooled, diluted with ethyl acetate, washed with water, brine and dried. Crude residue was column chromatographed to yield 94 in 30% yield.

¹H NMR (CDCl₃): 8.53 (brs, 1H), 7.61-7.58 (d, 2H, J=6 Hz), 740-7.38 (t, 1H, J=6H), 7.62-7.59 (d, 1H, J=6 Hz), 7.29-7.26 (d, 1H, J=6 Hz), 7.01-6.95 (m, 1H), 6.84-6.80 (m, 1H), 6.34-6.32 (d, 1H, J=6 Hz), 472-4.70 (d, 2H, J=6 Hz), 4.62-4.58 (m, 1H), 3.80 (s, 3H), 2.72 (s, 3H), 2.54-2.42 (m, 7H), 1.69-1.57 (m, 4H), 1.32-1.30 (d, 3H, J=6 Hz), 1.01-0.96 (t, 6H, J=7.2 Hz).

Mass spectrum (ESI⁺): m/z=464 [M+1].

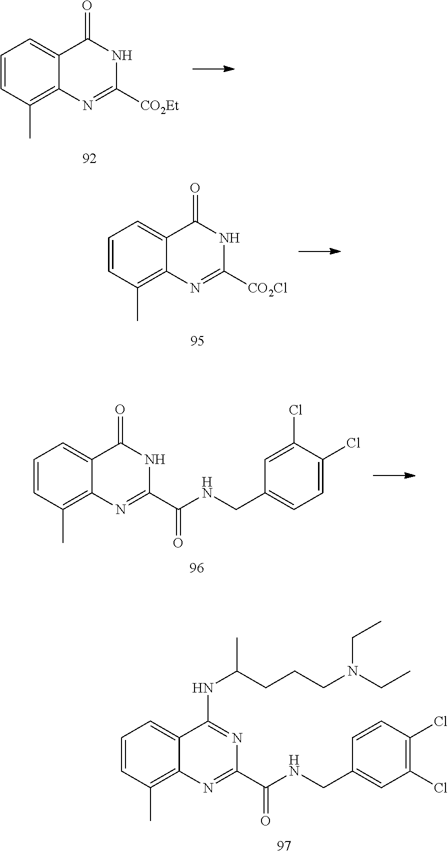

To the compound 92 (90 mg, 0.39 mmol) in THF (3 ml), 1N NaOH (1 ml) was added and stirred at room temperature for 3 h. Then reaction mixture was acidified with concentrated HCl to pH 2. Reaction mixture was diluted with water, and extracted with 10% MeOH/90% DCM. Organic layer was dried and evaporated. Crude material was carried to the next step.

To this crude in DCM, oxaloyl chloride was added followed by 1 drop of DMF. Reaction mixture was stirred for 24 h at room temperature. Volatiles were removed in vacuum and the crude was carried to the next step.

To the acid chloride 95 (70 mg, 0.31 mmol) in DCM, 3,4-dichloroaniline (84 mg, 0.46 mmol) and sodium bicarbonate (106 mg, 1.26 mmol) in water (4 ml) was added and stirred overnight at room temperature. Then reaction mixture was diluted with DCM, washed with water, brine and dried. Crude residue was column purified to yield 96 in 44% yield.

Mass spectrum (ESI⁺): m/z=362 [M+1].

To the solution of quinazolinone 96 (61 mg, 0.17 mmol) in DMF (3 ml), BOP (98 mg, 0.22 mmol) was added followed by DBU (38 mg, 0.25 mmol) and stirred for 20 min at room temperature. Then 2-amino-5-diethylaminopentane (42 mg, 0.25 mmol) was added and stirred for 15 min at room temperature and 6 h at 60° C. Then the reaction mixture was cooled, diluted with ethyl acetate, washed with water, brine and dried. Crude residue was column chromatographed to yield 97 in 40% yield.

¹H NMR (CDCl₃): 8.59 (brs, 1H), 7.62-7.58 (m, 2H), 7.50 (s, 1H), 741-7.37 (t, 2H, J=6H), 7.24 (s, 1H), 6.36 (br s, 1H), 4.68-4.66 (d, 2H, J=6 Hz), 2.73 (s, 3H), 2.71-2.41 (m, 7H), 1.73-1.58 (m, 4H), 1.32-1.30 (d, 3H, J=6 Hz), 1.01-0.96 (t, 6H, J=7.2 Hz).

Mass spectrum (ESI⁺): m/z=502 [M+1].

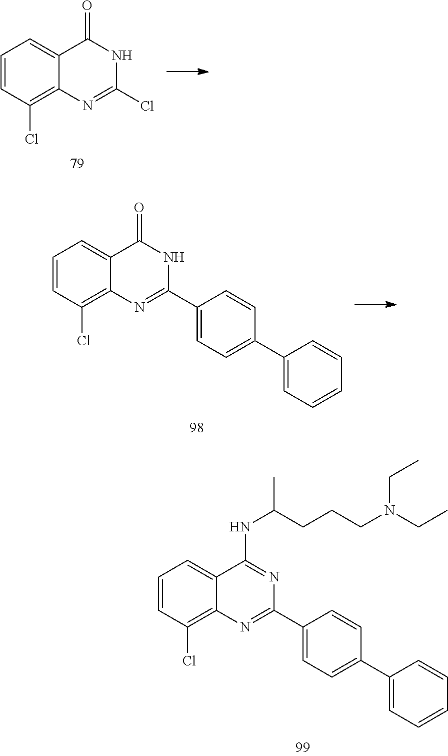 [SMILES: O=c1[nH]c(Cl)nc2c(Cl)cccc12 ; O=c1[nH]c(-c2ccc(-c3ccccc3)cc2)nc2c(Cl)cccc12 ; CCN(CC)CCCC(C)Nc1nc(-c2ccc(-c3ccccc3)cc2)nc2c(Cl)cccc12]

To the 2,8-dichloroquinazolin-4(3H) one 79 (70 mg, 0.33 mmol) in ethanol/toluene/water (2/5/2 ml), 1,1′ biphenyl-4-yl-boronic acid (49 mg, 0.40 mmol) and sodium acetate (136 mg, 1.66 mmol) were added followed by PdCl₂(dppf) (51 mg, 0.07 mmol). Then the reaction mixture was heated to 80° C. for 16 h. Then reaction mixture was cooled and diluted with ethyl acetate. Organic layer was separated and washed with water, brine and dried. Crude residue was column chromatographed to yield 98 in 65% yield.

Mass spectrum (ESI⁺): m/z=333 [M+1].

To the solution of quinazolinone 98 (60 mg, 0.18 mmol) in DMF (3 ml), BOP (103 mg, 0.23 mmol) was added followed by DBU (41 mg, 0.27 mmol) and stirred for 20 min at room temperature. Then 2-amino-5-diethylaminopentane (43 mg, 0.27 mmol) was added and stirred for 15 min at room temperature and 6 h at 60° C. Then the reaction mixture was cooled, diluted with ethyl acetate, washed with water, brine and dried. Crude residue was column chromatographed to yield 99 in 40% yield.

¹H NMR (CDCl₃): 8.71-8.68 (d, 2H, J=6 Hz), 7.82-7.67 (m, 5H), 7.47-7.26 (m, 5H), 6.43 (br s, 1H), 4.70 (br s, 1H), 2.66-2.58 (m, 7H), 1.86-1.72 (m, 4H), 1.42-1.40 (d, 3H, J=6 Hz), 1.07-1.02 (t, 6H, J=7.2 Hz).

Mass spectrum (ESI⁺): m/z=473 [M+1].

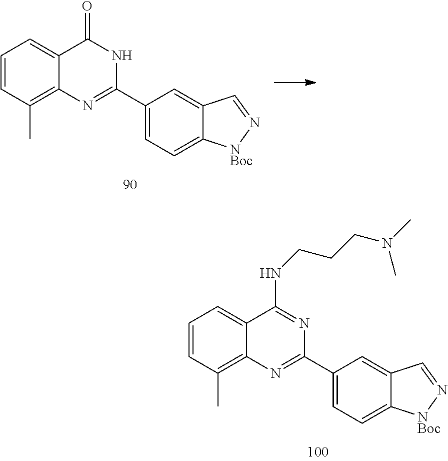 [SMILES: Cc1cccc2c(=O)[nH]c(-c3ccc4c(cnn4C(=O)OC(C)(C)C)c3)nc12 ; Cc1cccc2c(NCCCN(C)C)nc(-c3ccc4c(cnn4C(=O)OC(C)(C)C)c3)nc12]

To the solution of quinazolinone 90 (120 mg, 0.32 mmol) in DMF (3 ml), BOP (183 mg, 0.41 mmol) was added followed by DBU (73 mg, 0.48 mmol) and stirred for 20 min at room temperature. Then (3-aminopropyl) dimethylamine (52 mg, 0.51 mmol) was added and stirred for 15 min at room temperature and 6 h at 60° C. Then the reaction mixture was cooled, diluted with ethyl acetate, washed with water, brine and dried. Crude residue was column chromatographed to yield 100 in 50% yield.

¹H NMR (CDCl₃): 9.08 (s, 1H), 8.78-8.75 (d, 1H, J=6 Hz), 8.38 (br s, 1H), 8.19 (s, 1H), 8.21-8.18 (d, 1H, J=6 Hz) 7.56-7.46 (m, 3H), 7.29-7.26 (t, 1H, J=6 Hz), 5.16 (br s, 1H), 3.94-3.90 (m, 2H), 3.20-3.14 (m, 2H), 2.80 (s, 3H), 2.64-2.43 (m, 2H), 2.39-2.17 (m, 6H), 1.99-1.92 (m, 2H), 1.69-1.51 (m, 2H), 1.44 (s, 9H).

Mass spectrum (ESI⁺): m/z=461 [M+1].

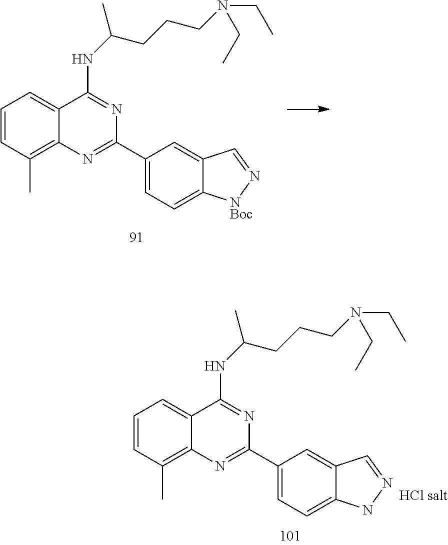

To the Boc compound 91 (12 mg, 0.023 mmol), 4N HCl in dioxane (2 ml) was added and stirred at room temperature for 5 h. Volatiles were removed in vacuum to yield 101 in quantitative yield.

¹H NMR (CDCl₃): 8.76 (s, 1H), 8.20 (s, 2H), 7.74-7.71 (m, 3H), 7.53 (s, 1H), 4.76-4.72 (m, 1H), 2.81 (s, 3H), 2.60-2.53 (m, 7H), 1.78-1.70 (m, 12H), 1.42-1.40 (d, 3H, J=6 Hz), 1.04-0.99 (t, 6H, J=7.2 Hz).

Mass spectrum (ESI⁺): m/z=452 [M+1].

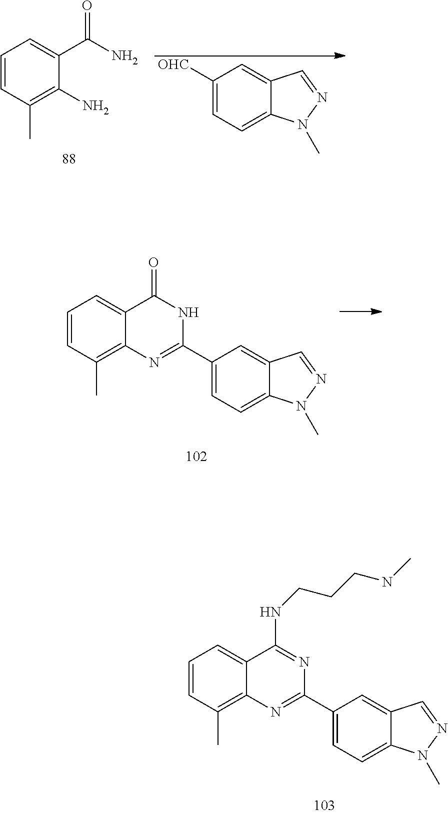 [SMILES: Cc1cccc(C(N)=O)c1N ; Cn1ncc2cc(C=O)ccc21 ; Cc1cccc2c(=O)[nH]c(-c3ccc4c(cnn4C)c3)nc12 ; CNCCCNc1nc(-c2ccc3c(cnn3C)c2)nc2c(C)cccc12]

To the amine 88 (250 mg, 1.67 mmol), 1-methyl-1H-indazole-5-carbaldehyde (267 mg, 1.67 mmol), iron(iii) chloride (108 mg, 0.67 mmol) was added followed by water (5 ml). Reaction mixture was then heated to 100° C. for 24 h. Then reaction mixture was cooled, filtered and the crude was carried to the next step.

To the solution of quinazolinone 102 (100 mg, 0.34 mmol) in DMF (3 ml), BOP (198 mg, 0.45 mmol) was added followed by DBU (78 mg, 0.51 mmol) and stirred for 20 min at room temperature. Then 2-amino-5-diethylaminopentane (52 mg, 0.51 mmol) was added and stirred for 15 min at room temperature and 6 h at 60° C. Then the reaction mixture was cooled, diluted with ethyl acetate, washed with water, brine and dried. Crude residue was column chromatographed to yield 103 in 50% yield.

¹H NMR (CDCl₃): 9.05 (s, 1H), 8.79-8.76 (d, 1H, J=6 Hz), 8.37 (br s, 1H), 8.10 (s, 1H), 7.56-7.43 (m, 3H), 7.29-7.26 (m, 1H), 4.12 (s, 3H), 3.96-3.91 (m, 2H), 2.81 (s, 3H), 2.65-2.61 (m, 2H), 2.40 (s, 6H), 1.96-1.92 (m, 2H).

Mass spectrum (ESI⁺): m/z=517 [M+1].

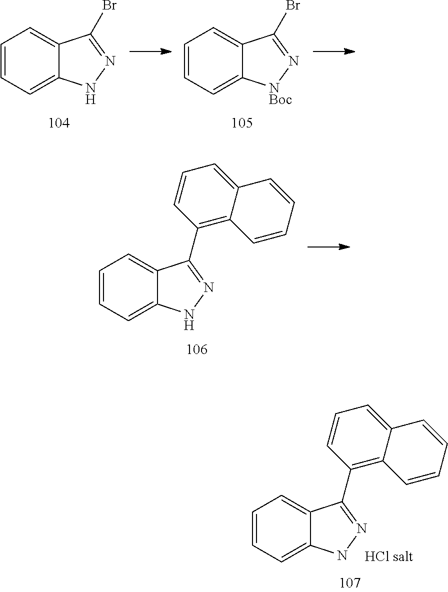 [SMILES: Brc1n[nH]c2ccccc12 ; CC(C)(C)OC(=O)n1nc(Br)c2ccccc21 ; c1ccc2c(-c3n[nH]c4ccccc34)cccc2c1 ; c1ccc2c(-c3n[nH]c4ccccc34)cccc2c1]

To the indazole 104 (3.0 g, 15.22 mmol) in THF (10 ml), DMAP (186 mg, 1.52 mmol) and (Boc)₂O (3.64 g, 16.7 mmol) were added and stirred at room temperature for 3 h. Then the reaction mixture was diluted with water, extracted with ethyl acetate. Organic layer was washed with water, brine and dried. Crude residue was column chromatographed to yield 89% of 105.

Mass spectrum (ESI⁺): m/z=297 [M+1].

To the bromo compound 105 (100 mg, 0.34 mmol), in dioxane\water (3\1 ml), naphthylboronic acid (69 mg, 0.40 mmol), sodium acetate (132 mg, 1.61 mmol) and PdCl₂(dppf) (50 mg, 0.07 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 106 in 51% yield.

Mass spectrum (ESI⁺): m/z=345 [M+1].

To the Boc compound 106 (30 mg, 0.09 mmol), 4N HCl in dioxane (2 ml) was added and stirred at room temperature for 16 h. Volatiles were removed in vacuum to yield 107 in quantitative yield.

Mass spectrum (ESI⁺): m/z=280 [M+1].

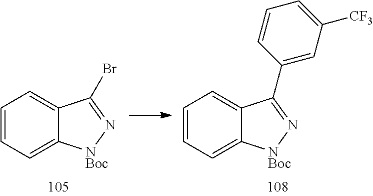 [SMILES: CC(C)(C)OC(=O)n1nc(Br)c2ccccc21 ; CC(C)(C)OC(=O)n1nc(-c2cccc(C(F)(F)F)c2)c2ccccc21]

To the bromo compound 105 (100 mg, 0.34 mmol), in dioxane\water (3\1 ml) 3-trifluoromethylphenylboronic acid (76 mg, 0.40 mmol), sodium acetate (132 mg, 1.61 mmol) and PdCl₂(dppf) (50 mg, 0.07 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 108 in 63% yield.

¹H NMR (CDCl₃): 8.28-8.19 (m, 3H), 7.97-7.94 (d, 1H, J=6 Hz), 7.75-7.56 (m, 3H), 7.43-7.38 t, 1H, J=7.8 Hz), 1.76 (s, 9H).

Mass spectrum (ESI⁺): m/z=363 [M+1].

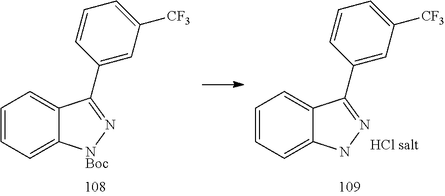

To the Boc compound 108 (40 mg, 0.11 mmol), 4N HCl in dioxane (2 ml) was added and stirred at room temperature for 16 h. Volatiles were removed in vacuum to yield 109 in quantitative yield.

¹H NMR (CDCl₃): 10.76 (br s, 1H), 8.27-7.99 (m, 5H), 7.63-7.43 (m, 2H), 7.40-7.25 (m, 1H).

Mass spectrum (ESI⁺): m/z=298 [M+1].

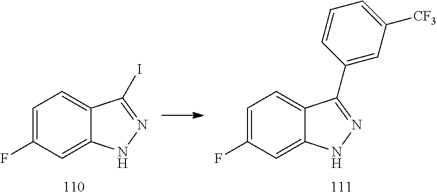 [SMILES: Fc1ccc2c(I)n[nH]c2c1 ; Fc1ccc2c(-c3cccc(C(F)(F)F)c3)n[nH]c2c1]

To the iodo compound 110 (78 mg, 0.30 mmol), in dioxane\water (3\1 ml), 3(trifluoromethyl)phenylboronic acid (68 mg, 0.36 mmol), sodium acetate (117 mg, 1.42 mmol) and PdCl₂(dppf) (44 mg, 0.06 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 111 in 72% yield.

¹H NMR (CDCl₃): 10.41 (br s, 1H), 8.1 (s, 1H), 8.14-8.12 (d, 1H, J=6 Hz), 7.96-7.92 (m, 1H), 7.70-7.61 (m, 2H), 7.16-7.13 (d, 1H, J=6 Hz), 7.05-7.02 (t, 1H, j=6 Hz).

Mass spectrum (ESI⁺): m/z=281 [M+1].

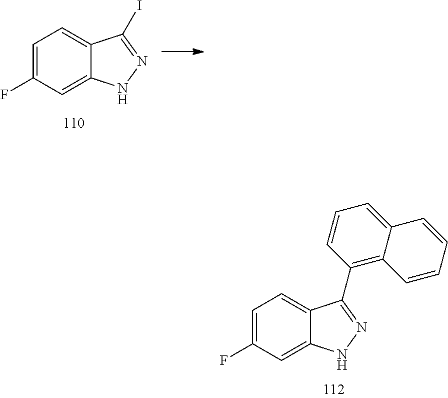 [SMILES: Fc1ccc2c(I)n[nH]c2c1 ; Fc1ccc2c(-c3cccc4ccccc34)n[nH]c2c1]

To the iodo compound 110 (78 mg, 0.30 mmol), in dioxane\water (3\1 ml), naphthylboronic acid (62 mg, 0.36 mmol), sodium acetate (117 mg, 1.42 mmol) and PdCl₂(dppf) (44 mg, 0.06 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 112 in 64% yield.

¹H NMR (CDCl₃): 8.38 (s, 1H), 8.10-7.89 (m, 5H), 7.54-7.52 (d, 2H, J=6 Hz), 7.21-7.18 (d, 1H, j=6 Hz), 7.04-7.01 (t, 2H, j=6 Hz).

Mass spectrum (ESI⁺): m/z=263 [M+1].

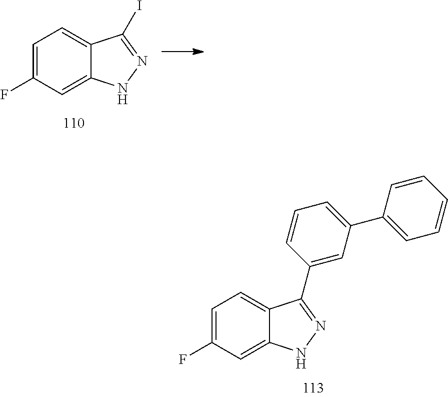 [SMILES: Fc1ccc2c(I)n[nH]c2c1 ; Fc1ccc2c(-c3cccc(-c4ccccc4)c3)n[nH]c2c1]

To the iodo compound 110 (78 mg, 0.30 mmol), in dioxane\water (3\1 ml), 3-biphenylboronic acid (71 mg, 0.36 mmol), sodium acetate (117 mg, 1.42 mmol) and PdCl₂(dppf) (44 mg, 0.06 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 113 in 65% yield.

¹H NMR (CDCl₃): 10.87 (br s, 1H), 8.17 (s, 1H), 8.00-7.91 (m, 2H), 7.68-7.46 (m, 4H), 7.49-7.25 (m, 3H), 7.02-6.97 (m, 2H).

Mass spectrum (ESI⁺): m/z=289 [M+1].

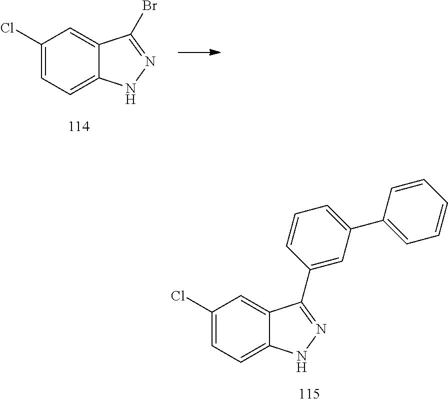 [SMILES: Clc1ccc2[nH]nc(Br)c2c1 ; Clc1ccc2[nH]nc(-c3cccc(-c4ccccc4)c3)c2c1]

To the bromo compound 114 (69 mg, 0.30 mmol), in dioxane\water (3\1 ml), 3-biphenylboronic acid (71 mg, 0.36 mmol), sodium acetate (117 mg, 1.42 mmol) and PdCl₂(dppf) (44 mg, 0.06 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 115 in 60% yield.

¹H NMR (CDCl₃): 10.22 (br s, 1H), 8.20 (s, 1H), 8.13-8.10 (d, 2H, J=6 Hz), 7.98 (s, 1H), 7.70-7.65 (m, 4H), 7.48-7.26 (m, 4H).

Mass spectrum (ESI⁺): m/z=305 [M+1].

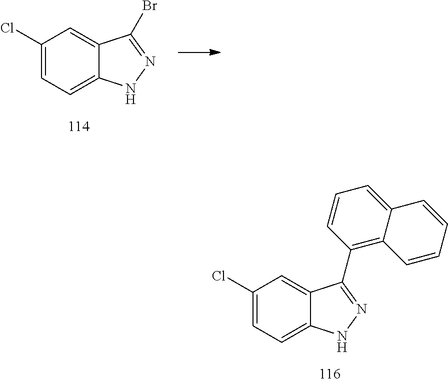 [SMILES: Clc1ccc2[nH]nc(Br)c2c1 ; Clc1ccc2[nH]nc(-c3cccc4ccccc34)c2c1]

To the bromo compound 114 (69 mg, 0.30 mmol), in dioxane\water (3\1 ml), naphthylboronic acid (62 mg, 0.36 mmol), sodium acetate (117 mg, 1.42 mmol) and PdCl₂(dppf) (44 mg, 0.06 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 116 in 55% yield.

¹H NMR (CDCl₃): 8.38 (s, 1H), 8.12-7.89 (m, 5H), 7.57-7.39 (m, 2H), 7.51-7.39 (m, 2H).

Mass spectrum (ESI⁺): m/z=279 [M+1].

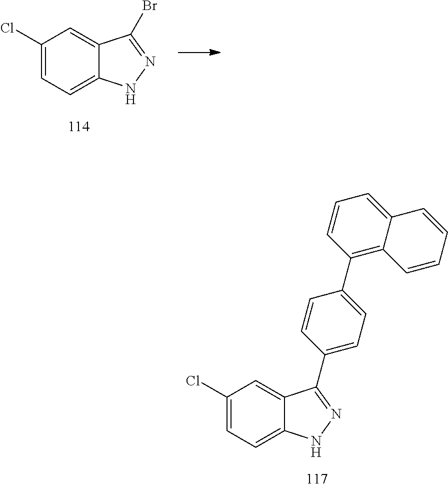 [SMILES: Clc1ccc2[nH]nc(Br)c2c1 ; Clc1ccc2[nH]nc(-c3ccc(-c4cccc5ccccc45)cc3)c2c1]

To the bromo compound 114 (65 mg, 0.30 mmol), in dioxane\water (3\1 ml), 4-(2-naphthyl)phenylboronic acid (89 mg, 0.36 mmol), sodium acetate (117 mg, 1.42 mmol) and PdCl₂(dppf) (44 mg, 0.06 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 117 in 48% yield.

¹H NMR (CDCl₃): 8.13 (s, 1H), 8.08-8.05 (d, 3H, J=6 Hz), 7.97-7.74 (m, 5H), 7.53-7.26 (m, 5H).

Mass spectrum (ESI⁺): m/z=355 [M+1].

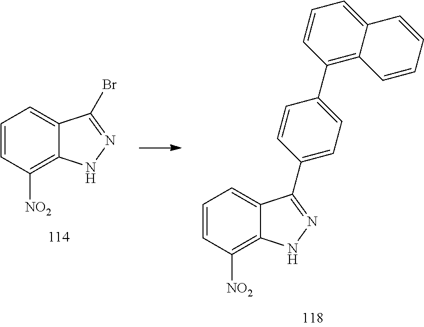 [SMILES: O=[N+]([O-])c1cccc2c(Br)n[nH]c12 ; O=[N+]([O-])c1cccc2c(-c3ccc(-c4cccc5ccccc45)cc3)n[nH]c12]

To the nitro compound 114 (72 mg, 0.30 mmol), in dioxane\water (3\1 ml), 4-(2-naphthyl)phenylboronic acid (89 mg, 0.36 mmol), sodium acetate (117 mg, 1.42 mmol) and PdCl₂(dppf) (44 mg, 0.06 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 118 in 45% yield.

¹H NMR (CDCl₃): 8.49-8.41 (m, 2H), 8.14-8.08 (m, 4H), 7.74-7.62 (m, 4H), 7.34-7.26 (m, 4H).

Mass spectrum (ESI⁺): m/z=366 [M+1].

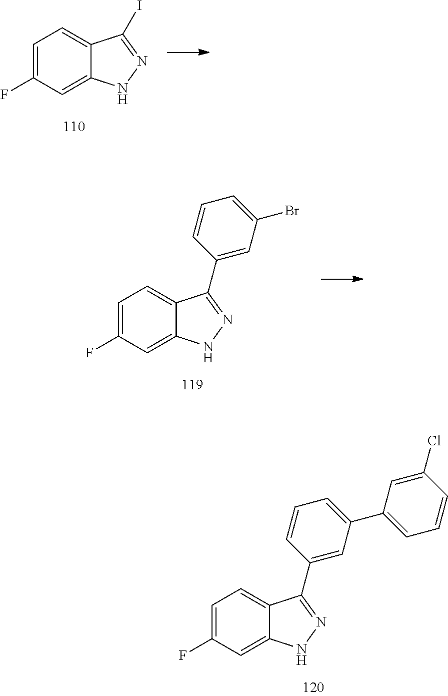 [SMILES: Fc1ccc2c(I)n[nH]c2c1 ; Fc1ccc2c(-c3cccc(Br)c3)n[nH]c2c1 ; Fc1ccc2c(-c3cccc(-c4cccc(Cl)c4)c3)n[nH]c2c1]

To the iodo compound 110 (400 mg, 1.53 mmol), in dioxane\water (103 ml), 3-bromophenylboronic acid (369 mg, 1.83 mmol), sodium acetate (591 mg, 7.20 mmol) and PdCl₂(dppf) (224 mg, 0.31 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 119 in 15% yield.

Mass spectrum (ESI⁺): m/z=291 [M+1].

To the bromo compound 119 (60 mg, 0.21 mmol), in dioxane\water (3\1 ml), 3-chlorophenylboronic acid (39 mg, 0.25 mmol), sodium acetate (82 mg, 0.99 mmol) and PdCl₂(dppf) (31 mg, 0.04 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 120 in 30% yield.

¹H NMR (CDCl₃): 8.13 (s, 1H), 7.95-7.66 (m, 2H), 7.64-7.33 (m, 6H), 6.9-6.96 (t, 1H, J=6 Hz), 6.76-6.75 (s, 1H, J=1.8 Hz).

Mass spectrum (ESI⁺): m/z=323 [M+1].

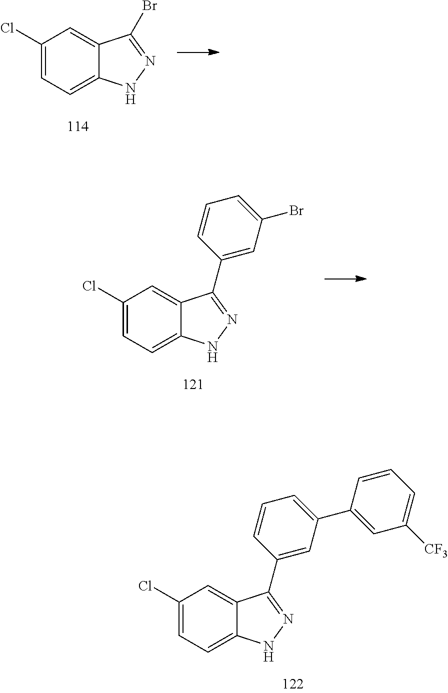 [SMILES: Clc1ccc2[nH]nc(Br)c2c1 ; Clc1ccc2[nH]nc(-c3cccc(Br)c3)c2c1 ; FC(F)(F)c1cccc(-c2cccc(-c3n[nH]c4ccc(Cl)cc34)c2)c1]

To the bromo compound 114 (320 mg, 1.38 mmol), in dioxane\water (6\2 ml), 3-bromophenylboronic acid (333 mg, 1.66 mmol), sodium acetate (536 mg, 6.54 mmol) and PdCl₂(dppf) (202 mg, 0.28 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 121 in 40% yield.

Mass spectrum (ESI⁺): m/z=307 [M+1].

To the bromo compound 121 (62 mg, 0.20 mmol), in dioxane\water (3\1 ml), 3-trifluoromethylphenylboronic acid (46 mg, 0.24 mmol), sodium acetate (78 mg, 0.95 mmol) and PdCl₂(dppf) (29 mg, 0.04 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 122 in 32% yield.

¹H NMR (CDCl₃): 8.47 (s, 1H), 8.24-8.20 (m, 1H), 7.96-7.28 (m, 14H).

Mass spectrum (ESI⁺): m/z=373 [M+1].

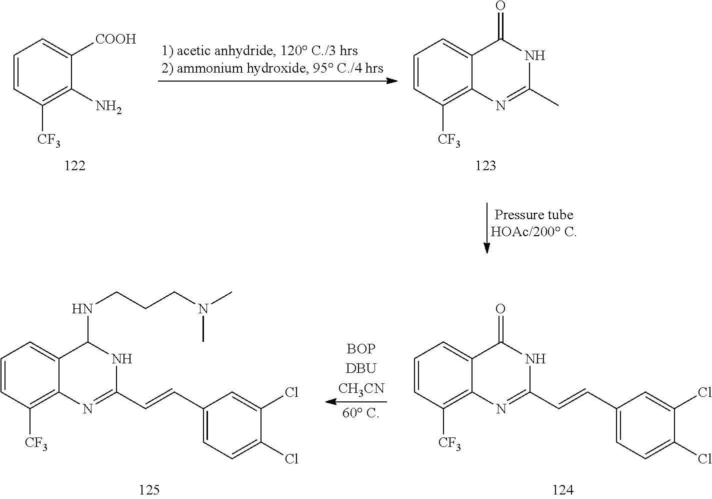 [SMILES: Nc1c(C(=O)O)cccc1C(F)(F)F ; Cc1nc2c(C(F)(F)F)cccc2c(=O)[nH]1 ; CN(C)CCCNC1NC(/C=C/c2ccc(Cl)c(Cl)c2)=Nc2c1cccc2C(F)(F)F ; O=c1[nH]c(/C=C/c2ccc(Cl)c(Cl)c2)nc2c(C(F)(F)F)cccc12]

To compound 122 (10 g, 0.0487 mol) was added acetic anhydride (27 g, 25 ml, 0.254 mol) and the mixture warmed to 120° C. for 3 h with stirring. The mixture was then concentrated in vacuo (50° C.) to remove excess acetic anhydride (bp 138° C.) to give a dry solid. Ammonium hydroxide (28% NH₃, 100 ml) was added and the mixture heated to 95° C. for 4 hrs. The mixture was cooled, vacuum filtered and the resultant solid washed with water, saturated NaHCO₃ solution and more water. The obtained solid was dried under vacuum to give 7.37 g of 2-methyl-8-(trifluoromethyl)quinazolin-4(3H)-one (compound 123) (66%).

¹H NMR (DMSO-d₆, 400 MHz): δ 12.5 (bs, 1H), δ 8.35 (d, J=8.0 Hz, 1H), δ 8.14 (d, J=7.4 Hz, 1H), δ 7.56-7.60 (m, 1H), δ 2.40 (s, 3H).

2-methyl-8-(trifluoromethyl)quinazolin-4(3H)-one (2.0 g, 8.76 mmol) was placed in a glass pressure tube. 3,4-dichlorobenzaldehyde (1.69 g, 9.64 mmol) was added and acetic acid (40 ml) was added to the mixture. The mixture was heated in an oil bath (200° C.) for 36 hrs. The reaction was cooled and the precipitate was vacuum filtered, washed with water, saturated NaHCO₃ solution, water and a small amount of diethyl ether to remove the excess aldehyde. The solid was dried in a vacuum desiccator to give 2.37 grams of the styryl quinazolinone product (E)-2-(3,4-dichlorostyryl)-8-(trifluoromethyl)quinazolin-4(3H)-one (compound 124) (70%).

¹H NMR (DMSO-d₆, 400 MHz): δ (12.6 (bs, 1H), δ 8.37 (d, J=7.8 Hz, 1H), δ 8.17 (d, J=7.0 Hz, 1H), δ 7.95 (s, 1 Hz), δ 7.87 (d, J=16.0 Hz, 1H), δ 7.7 (d, J=8.2 Hz, 1H), δ 7.59-7.66 (m, 2H), δ 7.07 (d, J=16.0 Hz, 1H).

(E)-2-(3,4-dichlorostyryl)-8-(trifluoromethyl)quinazolin-4(3H)-one (1.0 g, 2.596 mmol) and BOP (Benzotriazol-1-yloxy)tris(dimethylamino)phosphonium hexafluorophosphate) (1.493 g, 3.375 mmol) was stirred with acetonitrile (20 ml). DBU (1,8-Diazabicyclo[5.4.0]undec-7-ene) (593 mg, 0.583 ml, 3.894 mmol) was added to the mixture and stirred 10-15 minutes. The amine 3-(dimethylamino)-1-propylamine (398 mg, 0.457 ml, 3.894 mmol) was added and the mixture was heated to 70° C. for 3 hours and then stirred at room temperature overnight. The solvent was removed in vacuo and the residue partitioned with EtOAc and brine. The organic layer was dried (Na₂SO₄), filtered and concentrated in vacuo. Purification by silica flash chromatography (9:1 DCM:MeOH) provided 500 mg of the expected product (E)-N¹-(2-(3,4-dichlorostyryl)-8-(trifluoromethyl)quinazolin-4-yl)-N³,N³-dimethylpropane-1,3-diamine (compound 125) (39%).

¹H NMR (DMSO-d₆, 400 Mz): δ 8.59 (m, 1H), δ 8.46 (d, J=7.8 Hz, 1H), δ 8.12 (d, J=7.42 Hz, 1H), δ 8.05 (s, 1H), δ 7.90 (d, J=16.0 Hz, 1H), δ 7.72-7.75 (m, 1H), δ 7.65 (d, J=8.2 Hz, 1H), δ 7.56-7.60 (m, 1H), δ 7.20 (d, J=16.0 Hz, 1H), δ 3.70 (q, 2H), δ 2.40 (t, 2H), δ 1.85 (m, 2H).

Mass spectrum (ESI⁺): m/z=469 [M+1].

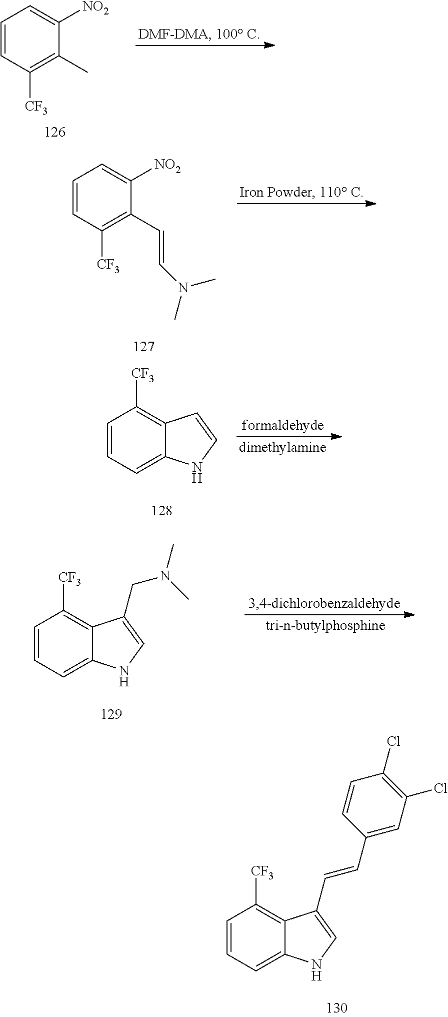 [SMILES: Cc1c([N+](=O)[O-])cccc1C(F)(F)F ; CN(C)/C=C/c1c([N+](=O)[O-])cccc1C(F)(F)F ; FC(F)(F)c1cccc2[nH]ccc12 ; CN(C)Cc1c[nH]c2cccc(C(F)(F)F)c12 ; FC(F)(F)c1cccc2[nH]cc(/C=C/c3ccc(Cl)c(Cl)c3)c12]

Under an atmosphere of argon, a solution of 6-trifluoromethyl-2-nitrotoluene (compound 126) (5.0 g, 24 mmol) in DMF (30 ml) was treated with N,N-dimethylformamide dimethyl acetal (16 g, 18 ml, 135 mmol). The mixture was heated to 100° C. and maintained for 6 hours. After cooling, the mixture was poured into ice-water and extracted with EtOAc and the organic layer washed with sat'd NaHCO₃ solution, NH₄Cl solution, LiCl solution, and brine. The organic extract was dried (MgSO₄) and concentrated in vacuo. The residue was solubilized in hexane and passed through a pre-equilibrated pad of SiO₂ eluting with 95/5 Hexane/EtOAc. The product (compound 127) was further purified by SiO₂ flash chromatography (98/2 Hexane/EtOAc).

The intermediated enamine (127) was dissolved in acetic acid (150 ml) and iron powder (4.3 g) was added and the mixture was heated to 110° C. under an argon atmosphere overnight (16 h). The mixture as cooled and 2N HCl was added to dissolve the mixture. The organic layer was neutralized with a saturated solution of K₂CO₃ and the mixture extracted with EtOAc. The organic layer was dried (Na₂SO₄) and subsequently purified by SiO₂ flash chromatography (95/5 Hexane/EtOAc) to give 0.77 g of the desired product 4-(trifluoromethyl)-1H-Indole (17%) (compound 128).

¹H NMR (DMSO-d₆, 400 MHz): δ 11.64 (bs, 1H), δ 7.7 (d, J=8.2 Hz, 1H), δ 7.58-7.59 (m, 1H), δ 7.37 (d, J=7.2 Hz, 1H), δ 7.23-7.27 (m, 1H), δ 6.5 (d, J=1.37 Hz, 1H).

Mass spectrum (ESI⁺): m/z=186 [M+1].

A solution of aqueous formaldehyde (37%, 0.465 ml, 6.24 mmol) and dimethylamine (40%, 0.79 ml, 6.25 mmol) in 10 ml EtOH was cooled to 0° C. 4-trifluoromethylindole (0.77 g, 4.16 mmol) (compound 128) was dissolved in a HOAc:EtOH mixture (1:1, 10 ml) and added dropwise to the reaction mixture. After stirring at this temperature for 2 hrs, the mixture was allowed to warm to room temperature and stirred overnight.

The mixture was cautiously added to a sat'd solution of NaHCO₃. 1N NaOH was added until the pH was between 9-10. The resulting mixture was extracted with CH₂Cl₂ (3×). The organic extracts were combined and washed with brine, dried over MgSO₄, filtered and concentrated in vacuo to give 1.0 g of N,N-dimethyl-1-(4-(trifluoromethyl)-1H-indol-3-yl)methanamine (compound 129). This was used as-is without further purification.

The crude 4-trifluoromethylgramine (1.0 g, 4.12 mmol) was dissolved in acetonitrile (20 ml). Under an argon atmosphere was added 3,4-dichlorobenzaldehyde (1.08 g, 6.18 mmol) and then added tri-n-butylphosphine (1.25 g, 1.5 ml, 6.18 mmol). The mixture was warmed to 78° C. overnight (16 hrs). Cool and concentrate in vacuo. The residue was taken up in a mixture of Hexane/EtOAc (9:1, 20 ml) and passed through a pre-equilibrated pad of SiO₂ and eluted with 9:1 Hexane/EtOAc to give a partially purified product. Further purification by SiO₂ flash chromatography eluting with 95/5 Hexane/EtOAc) provided 382 mg of the desired (E)-3-(3,4-dichlorostyryl)-4-(trifluoromethyl)-1H-indole (compound 130) (26%).

¹H NMR (DMSO-d₆, 400 MHz): δ 12.0 (bs, 1H), d 8.05 (d, 2.73 Hz, 1H), d 7.76 (d, J=8.2 Hz, 1H), d 7.60-7.68 (m, 2H), d 7.40-7.49 (m, 3H), d 7.29 (m, 1H), d 6.99 (d, J=16 Hz, 1H).

¹⁹F NMR (DMSO-d₆, 400 MHz): one peak d-57.20 ppm (singlet).

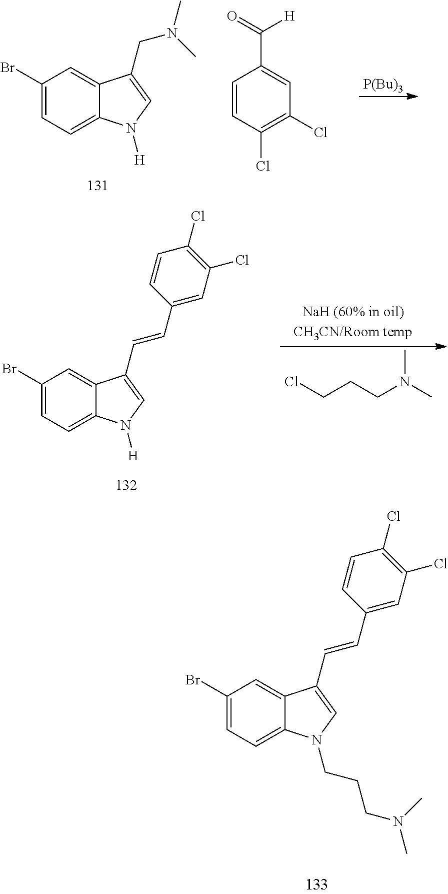

The commercially available 5-bromo-3-[(dimethylamino)methyl]indole, bromogramine) (131) (1.02 g, 3.95 mmol, 1 equiv) was dissolved in 10-15 ml acetonitrile. To this was added 3,4-dichlorobenzaldehyde (1.04 g, 5.93 mmol, 1.5 equiv). The flask was flushed with argon and tri-n-butylphosphine was added (1.2 grams (1.48 ml), 5.93 mmol, 1.5 equiv). This mixture was warmed to 80° C. for 24 hours. Cool and concentrate in vacuo (bath temp 30° C.). To the residue was added 50 ml 80/20 hexane:EtOAc and this was passed through a prepared pad of SiO₂ in a medium frit buchner funnel and eluted with 80/20 hexane: EtOAc. The cleaned product was isolated and further purified by SiO₂ flash chromatography (99/1 hexane: EtOAc) to give 0.4 grams of the desired product (compound 132) (28%).

¹H NMR (DMSO-d₆, 400 MHz) δ 11.60 (bs, 1H), d 8.24 (d, J=2 Hz, 1H), d 7.92 (d, J=2 Hz, 1H), d 7.75 (s, 1H), d 7.52-7.62 (m, 3H), d 7.4 (d, 8.8 Hz, 1H), d 7.28 (dd, J=8.8 Hz, J=2 Hz, 1H), d 7.6 (d, J=16.4 Hz, 1H).

The indole 132 (400 mg, 1.089 mmol) was dissolved in acetonitrile (20 ml). Sodium hydride (60% in oil, 148 mg, 3.7 mmol) and stirred 30 min. The 3-chloro-N,N-dimethylpropan-1-amine (480 mg, 3.9 mmol) was added and heated to 50° C. for 2 hrs. The solvent was then evaporated in vacuo and the residue partitioned with EtOAc and water, dried (Na₂SO₄), filtered and evaporated in vacuo. Purification with Si-gel chromatography (9; 1 DCM/MeOH) provided a mixture of the cis and trans isomers. Further purification via silica gel chromatography (9:1 EtOAc/MeOH) provided the desired trans product (compound 133). ¹H NMR (CDCl₃, 400 MHz): 1.95 (m, 2H), 2.2 (m, 8H, overlapping peaks), 4.15 (m, 2H), 6.85 (d, J=16.2 Hz, 1H), 7.16 (d, J=16.2 Hz, 1H), 7.22-7.3 (m, 3H), 7.32-7.4 (m, 2H), 7.54 (s, 1H), 8.03 (s, 1H).

Mass spectrum (ESI⁺): m/z=451 [M+1].

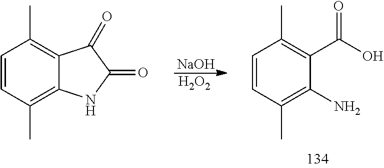

A 100 mL round bottom flask was charged with NaOH (0.9133 g, 22.8 mmol, 4 eq) and a stir bar. Water (23 mL) was added. 4,7-dimethyl-2,3-dihydro-1H-indole-2,3-dion (Enamine, 1.0 g, 5.71 mmol, 1 eq) was added to the resulting stirred solution. H₂O₂(30% by wt, 1.62 g, 14.3 mmol, 2.5 eq) was added dropwise. After 3 h TLC indicated complete reaction. The mixture was cautiously adjusted to pH≈2 with HCl (foaming). The mixture was filtered, rinsing with water. The filtrate was extracted with EtOAc (×2). The combined organics were washed with water (×2), brine (×1), and dried over Na₂SO₄. The solids were filtered off, and the volatiles were removed via rotary evaporation. The resulting solid was triturated with 5% DCM in hexanes and collected via vacuum filtration. The filter cake was triturated with 10% EtOAc in hexanes (×1), hexanes (×1), and dried. 0.504 g (3.1 mmol, 54% yield) of 134 was isolated as a tan solid. Mass spectrum (ESI⁺): m/z=166 [M+1]observed.

A 25 mL round bottom flask was charged with 2 (0.11 g, 0.60 mmol) and a stir bar. EtOAc (10 mL) was added, and the flask was purged with Ar. 10% Pd/C (0.015 g, z 15% by wt.) was added. The resulting mixture was stirred vigorously under H₂ (balloon pressure). After 90 min the mixture was filtered through Celite. The filter cake was rinsed with EtOAc, 10% MeOH in EtOAc, and 20% MeOH in DCM until no additional product could be detected by TLC in the filtrate. The filtrate was concentrated yielding 0.0922 g (0.61 mmol, ≈100% yield) of 3 as a yellow solid. Mass spectrum (ESI⁺): m/z=153 [M+1]observed.

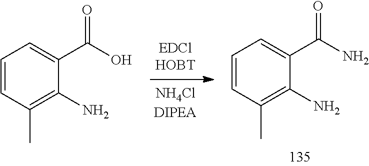

A 500 mL round bottom flask was charged with 2-amino-3-methylbenzoic acid (Combi-Blocks, 4.0 g, 26.5 mmol, 1 eq), NH₄Cl (4.25 g, 79.4 mmol, 3 eq) and a stir bar. The flask was evacuated and back-filled with Ar (×3). Anhydrous DCM (100 mL) and anhydrous DMF (20 mL) were added. The resulting stirred mixture was cooled to 0° C. 1-hydroxybenzotriazole hydrate (3.93 g, 29.1 mmol, 1.1 eq) was added followed by 1-(3-dimethylaminopropyl)-3-ethylcarbodiimide hydrochloride (5.56 g, 29.1 mmol, 1.1 eq) 5 min later. After 1 h DIPEA (32 mL, 23.9 g, 185 mmol, 7 eq) was added dropwise. The reaction was stirred at 0° C. to room temperature overnight. The volatiles were removed via rotary evaporation. The residue was diluted with water and adjusted to pH≈8-9 with conc. NH₃(aq). The resulting mixture was extracted with EtOAc (×3). The combined organics were dried over Na₂SO₄. The solids were filtered off, and the volatiles were removed via rotary evaporation. The residue was diluted with DCM and the resulting mixture was diluted with hexanes. The solids were collected via vacuum filtration. The filter cake was triturated with 10% DCM in hexanes and dried. 2.56 g (17.0 mmol, 64% yield) of 135 was collected as an off-white solid. Mass spectrum (ESI⁺): m/z=151 [M+1]observed.

The following anthranilamides were synthesized in a manner similar to that described for 135 above using either DMF or DCM/DMF as solvent.

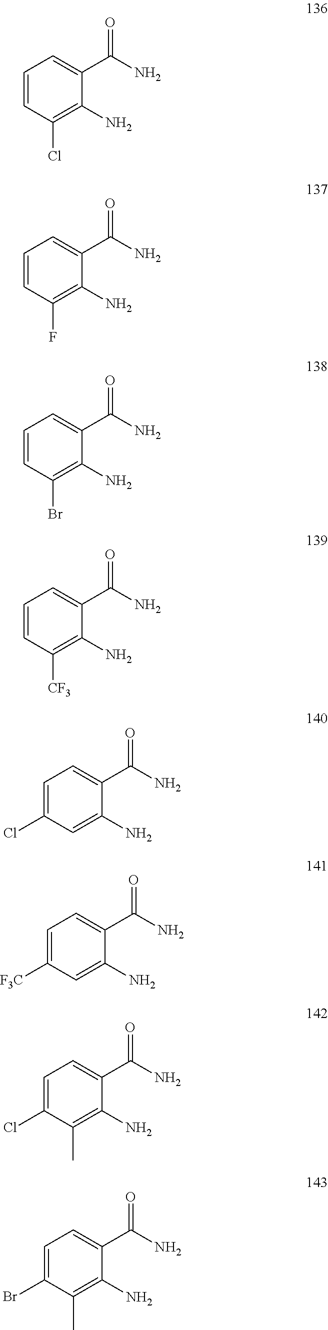

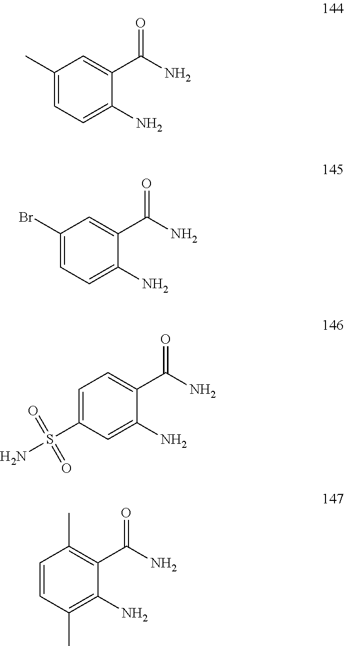

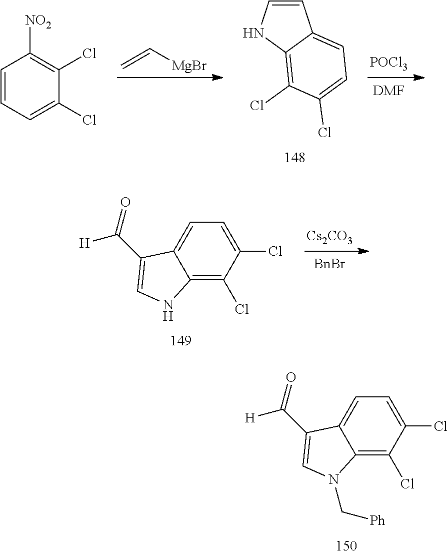

An oven dried 100 mL, pear-shaped flask, stir bar, and addition funnel were assembled and cooled to room temperature under vacuum. 2,3-Dichloronitrobenzene (Combi-Blocks, 1.25 g, 6.51 mmol, 1 eq) was added. The system was evacuated and back-filled with Ar (×3). Anhydrous THF (50 mL) was added, and the resulting stirred solution was cooled to −40° C. Vinylmagnesium bromide (Acros, 0.7M in THF, 30 mL, 20.8 mmol, 3.2 eq) was added dropwise via the addition funnel. After 70 min at temperature the reaction was quenched with NH₄Cl (aq, saturated). The resulting mixture was extracted with MTBE (×1), and the layers were separated. The organic layer was washed with water (×1), brine (×1), and dried over Na₂SO₄. The solids were filtered off and silica gel (13 g) was added. The volatiles were removed via rotary evaporation. Purification via flash chromatography on silica gel eluting with 0-10% EtOAc in hexanes yielded 0.6928 g (3.7 mmol, 57% yield) of 148 as an orange oil which slowly solidified.

A 2-necked, 25 mL round bottom flask was equipped with a stir bar and addition funnel. The system was evacuated and back-filled with Ar (×3). Anhydrous DMF (4 mL) was added to the flask. After cooling to 0° C. POCl₃ (0.38 mL, 0.628 g, 4.10 mmol, 1.1 eq) was added dropwise with stirring. After 10 min the cooling bath was removed. A solution of 148 (0.6928 g, 3.7 mmol, 1 eq) in anhydrous DMF (11 mL) was added dropwise via the addition funnel. After 3 h the mixture was poured into ice/NaHCO₃ (aq, saturated). The resulting mixture was extracted with EtOAc (×2). The combined organics were washed with water (×2), brine (×1), and dried over Na₂SO₄. The solids were filtered off, and the volatiles were removed via rotary evaporation. Trituration of the resulting solid with 10% MTBE in hexanes yielded 0.539 g (2.5 mmol, 68% yield) of 149 as a pale orange solid. Mass spectrum (ESI⁺): m/z=214 [M+1]observed.

A 50 mL round bottom with stir bar was charged with 149 (0.25 g, 1.2 mmol, 1 eq). The flask was evacuated and back-filled with Ar (×3). Anhydrous DMF (7 mL) was added followed by benzyl bromide (0.16 mL, 0.2297 g, 1.3 mmol, 1.15 eq). After 5 min Cs₂CO₃ (0.4186 g, 1.3 mmol, 1.1 eq) was added. The flask was covered in foil, and the reaction was stirred overnight. The reaction was diluted with water. After stirring for approx. 20 min the solids were collected via vacuum filtration. The filter cake was triturated with water (×2), 5% MTBE in hexanes (×2), and hexanes (×1). After drying 0.3226 g (1.1 mmol, 91% yield) of 150 was isolated as a pale yellow solid. Mass spectrum (ESI⁺): m/z=304 [M+1]observed.

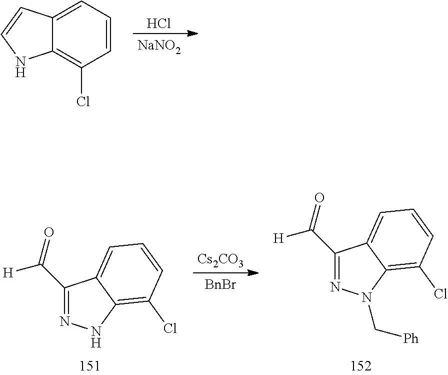

A 250 mL Erlenmeyer flask was charged with NaNO₂ (4.55 g, 66 mmol, 10 eq) and a stir bar. Water (130 mL) was added. 7-chloro-1H-indole (1.0 g, 6.6 mmol, 1 eq) was added followed by acetone (10 mL). The mixture was cooled to 0° C. with stirring. HCl (6N, 8.8 mL, 52.8 mmol, 8 eq) was added dropwise with vigorous stirring. After completion of the addition the cooling bath was removed, and the reaction was stirred overnight. The reaction was extracted with EtOAc, and the layers were separated. The organic layer was washed with water (×2), brine (×1), and dried over Na₂SO₄. The solids were filtered off. Silica gel (10 g) was added, and the volatiles were removed via rotary evaporation. Purification via flash chromatography on silica gel eluting with 0-45% EtOAc in hexanes yielded 0.3583 g (2.0 mmol, 30% yield) of 151 as a purple solid. Mass spectrum (ESI⁺): m/z=181 [M+1]observed.

151 was converted into 152 in a manner similar to that described for 150 above. Mass spectrum (ESI⁺): m/z=271 [M+1]observed.

The following N-substituted indoles were prepared in a manner similar to that described for 150 above.

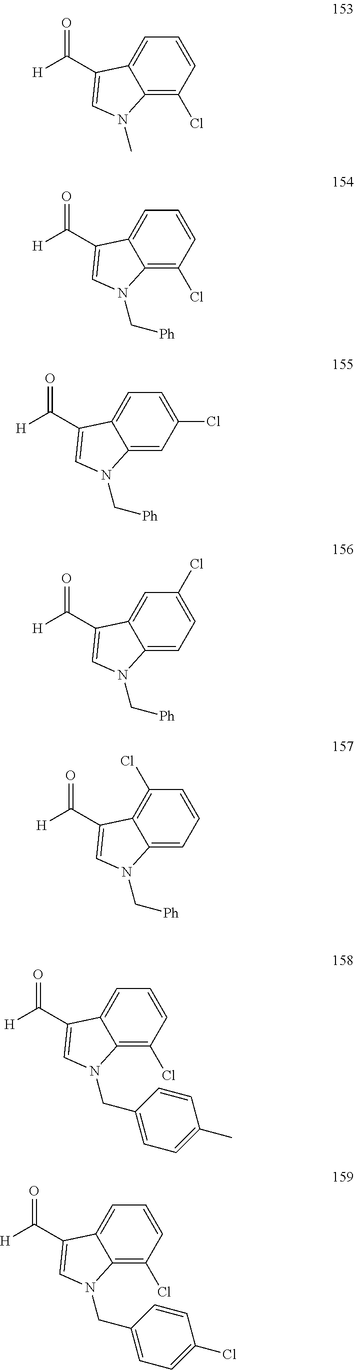

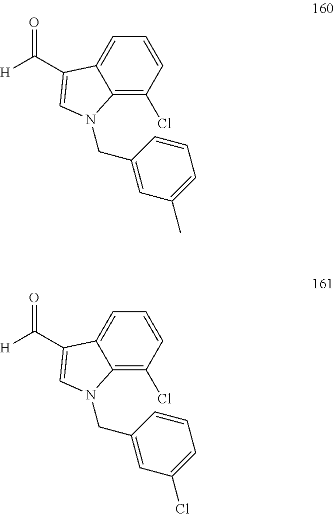 [SMILES: Cc1cccc(Cn2cc(C=O)c3cccc(Cl)c32)c1 ; O=Cc1cn(Cc2cccc(Cl)c2)c2c(Cl)cccc12]

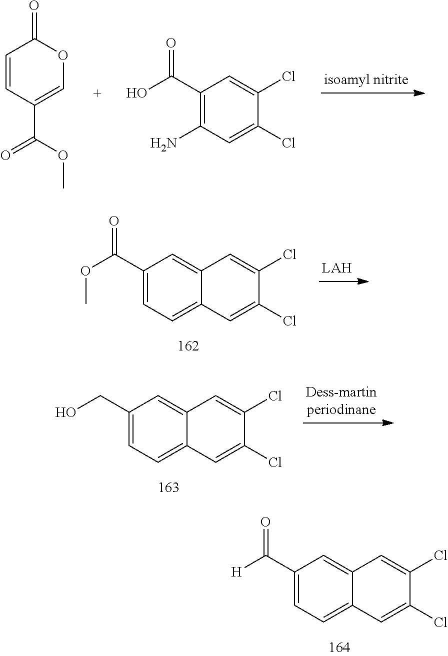 [SMILES: COC(=O)c1ccc(=O)oc1 ; Nc1cc(Cl)c(Cl)cc1C(=O)O ; COC(=O)c1ccc2cc(Cl)c(Cl)cc2c1 ; OCc1ccc2cc(Cl)c(Cl)cc2c1 ; O=Cc1ccc2cc(Cl)c(Cl)cc2c1]

An oven-dried 2-necked, 50 mL round bottom flask, stir bar, and reflux condenser were assembled and cooled to room temperature under vacuum. Methyl coumalate (Combi-Blocks, 0.3116 g, 2.0 mmol, 1 eq) and 2-amino-4,5-dichlorobenzoic acid (AstaTech, 0.50 g, 2.4 mmol, 1.2 eq) were added to the flask, and the system was evacuated and back-filled with Ar (×3). Anhydrous toluene (10 mL) was added, and the mixture was heated to 80° C. with stirring. Isoamyl nitrite (0.33 mL, 0.284 g, 2.4 mmol, 1.2 eq) was added via slow dropwise addition. The resulting red slurry was heated to reflux. After 2 h TLC indicated unreacted methyl coumalate was present. The reaction was returned to 80° C. and treated with additional isoamyl nitrite (0.33 mL). The reaction was returned to reflux. After 2 h TLC indicated no improvement in the reaction. The reaction was cooled to room temperature. The reaction was washed with 2N NaOH (×2), sodium bisulfite (aq, 5%, ×1), water (×1), 1N HCl (×1), water (×1), and dried over Na₂SO₄. The solids were filtered off, and the volatiles were removed via rotary evaporation. The solids were filtered off, and the volatiles were removed via rotary evaporation. Purification via flash chromatography on silica gel eluting with 0-10% EtOAc in hexanes yielded 0.090 g (0.35 mmol, 18% yield) of 162 as a yellow solid. Mass spectrum (ESI⁺): m/z=255 [M+1]observed.

A 50 mL round bottom flask was charged with 162 (0.090 g, 0.35 mmol, 1 eq) and a stir bar. The flask was evacuated and back-filled with Ar (×3). Anhydrous THF (10 mL) was added, and the resulting stirred solution was cooled to 0° C. LAH (2.0 M in THF, 0.19 mL, 0.37 mmol, 1.05 eq) was added dropwise. After 2 h at temperature the reaction was quenched by the addition of Na₂SO₄ (aq, saturated). The mixture was filtered through Celite, rinsing with EtOAc. The layers were separated. The organic layer was washed with brine (×1) and dried over Na₂SO₄. The solids were filtered off. Silica gel (2 g) was added, and the volatiles were removed via rotary evaporation. Purification via flash chromatography on silica gel eluting with 0-35% EtOAc in hexanes yielded 0.0734 g (0.32 mmol, 92% yield) of 163 as a white solid. Mass spectrum (ESI⁺): m/z=227 [M+1]observed.

A 50 mL round bottom flask was charged with 163 (0.0734 g, 0.32 mmol, 1 eq) and a stir bar. The flask was evacuated and back-filled with Ar (×3). Anhydrous DCM (8 mL) was added. The resulting stirred solution was treated with Dess-martin periodinane (0.1714 g, 0.4 mmol, 1.25 eq). After 45 min the reaction was quenched with NaHCO₃ (aq, saturated), and the layers were separated. The organic layer was dried over Na₂SO₄. The solids were filtered off, and the volatiles were removed via rotary evaporation yielding 0.0676 g (0.3 mmol, 94% yield) of 164. Mass spectrum (ESI⁺): m/z=225 [M+1]observed.

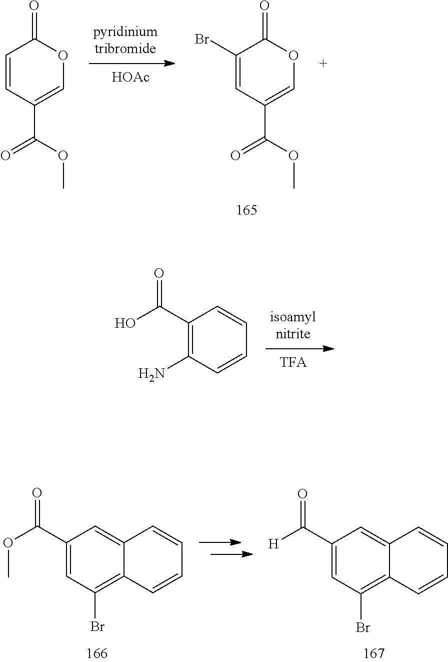

A 50 mL round bottom flask with addition funnel was charged with pyridinium tribromide (8.09 g, 25.3 mmol, 1.3 eq) and a stir bar. Methyl coumalate (3.0 g, 19.5 mmol, 1 eq) was added to the addition funnel. The system was evacuated and back-filled with Ar (×3). HOAc was added to the flask (17 mL) and addition funnel (10 mL). The reaction flask was heated to reflux. The methyl coumalate solution was added dropwise. After 4 h the reaction was cooled to room temperature and diluted with water. The solution was extracted with 1:1 EtOAc/hexanes, and the layers were separated. The organic layer was washed with water (×2), brine (×1), and dried over Na₂OS₄. The solids were filtered off, and the volatiles were removed via rotary evaporation. The resulting crude was cautiously diluted with NaHCO₃ (aq, saturated). The resulting solids were collected via vacuum filtration. The filter cake was triturated with additional NaHCO₃ and water. After drying 3.76 g (16.1 mmol, 83% yield) of 165 was isolated as a pale yellow solid. Mass spectrum (ESI⁺): m/z=233/235 [M+1]observed.

An oven dried 50 mL, 3-necked round bottom flask fitted with a reflux condenser and 2 addition funnels was cooled to room temperature under vacuum. The flask was charged 165 (0.750 g, 3.2 mmol, 1 eq) and a stir bar. Anthranilic acid (0.8828 g, 6.4 mmol, 2 eq) was added to one of the addition funnels. The system was evacuated and back-filled with Ar (×3). Isoamyl nitrite (0.86 mL, 0.754 g, 6.4 mmol, 2 eq) was added to the empty addition funnel. Anhydrous 1,2-DME (24 mL) was divided evenly among the flask and 2 addition funnels. TFA (25 μL, 0.0367 g, 0.32 mmol, 0.1 eq) was added to the flask, and the solution was heated to reflux. The anthranilic acid and isoamyl nitrite solutions were added dropwise at matching rates. The reaction was refluxed for 1 h following the completion of addition. The reaction was cooled to 50° C. and diluted with toluene. After cooling to room temperature the reaction was washed with 2N NaOH (×2), sodium bisulfite (aq, 5%, ×1), water (×1), 2N HCl (×1), water (×1), and dried over Na₂SO₄. The solids were filtered off, and the volatiles were removed via rotary evaporation. The residue was dissolved in DCM and silica gel (10 g) was added. The volatiles were removed via rotary evaporation. Purification via flash chromatography on silica gel eluting with 0-20% EtOAc in hexanes yielded 0.6684 g (2.5 mmol, 78% yield) of 166 as a yellow solid. Mass spectrum (ESI⁺): m/z=265/267 [M+1]observed.

166 was converted to 167 in a manner similar to that described above for 164. Mass spectrum (ESI⁺): m/z=236/238 [M+1]observed.

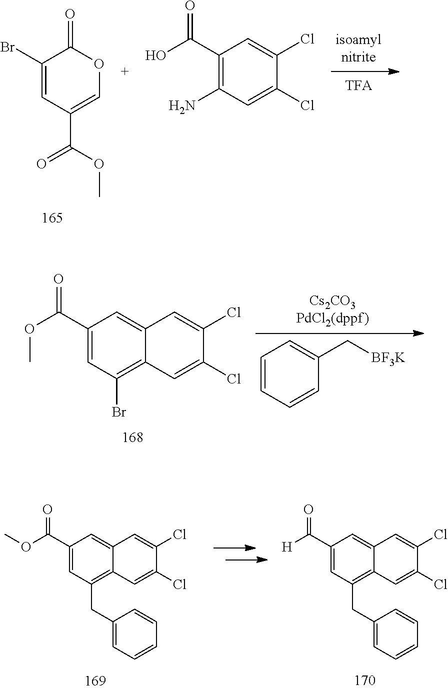

An oven-dried 2-necked, 50 mL round bottom flask equipped with stir bar and reflux condenser were cooled to room temperature under vacuum. The flask was charged with 2-amino-4,5-dichlorobenzoic acid (1.3262 g, 6.44 mmol, 1.5 eq) and 165 (1.0 g, 4.29 mmol, 1 eq). The system was evacuated and back-filled with Ar (×3). Anhydrous 1,2-DME (25 mL) was added followed by TFA (30 μL, 0.049 g, 0.43 mmol, 0.1 eq). The mixture was heated to reflux with stirring. Isoamyl nitrite (0.86 mL, 0.754 g, 6.44 mmol, 1.5 eq) was added dropwise. The reaction was refluxed for 90 min after the addition was complete before anhydrous toluene (10 mL) was added via rapid dropwise addition. After an additional 30 min at temperature the reaction was cooled to 50° C. Toluene (15 mL) was added, and the reaction was cooled to room temperature. The layers were separated. The organic layer was washed with 2N NaOH (×1), sodium bisulfite (aq, 5%, ×1), water (×1), 2N HCl (×1), and dried over Na₂SO₄. The solids were filtered off, and the volatiles were removed via rotary evaporation. The residue was dissolved in DCM/MeOH. DMSO (3 mL) was added, and the mixture was concentrated to remove the DCM and MeOH. Water (100 mL) was added, and the resulting solids were collected via vacuum filtration. The filter cake was triturated with water (×2), hexanes (×2), 20% toluene in hexanes (×2), and 10% DCM in hexanes (×2). After drying 0.581 g (1.74 mmol, 40% yield) of 168 was collected as a salmon colored solid. Mass spectrum (ESI⁺): m/z=333/335 [M+1]observed.

A 40 mL screw-top vial was charged with 168 (0.300 g, 0.90 mmol, 1 eq), potassium benzyltrifluoroborate (Combi-Blocks, 0.2223 g, 1.12 mmol, 1.25 eq), Cs₂CO₃ (0.878 g, 2.69 mmol, 3 eq), and a stir bar. 1,4-dioxane/water (10:1, 15 mL) was added. The vial was sealed with a septum, and the stirred mixture was sparged with Ar (10 min). PdCl₂(dppf) (0.0657 g, 0.09 mmol, 0.1 eq) was added. The vial was closed with the lid, and the reaction was heated to 90° C. overnight. After cooling to room temperature the reaction was diluted with water/EtOAc. The layers were separated. The organic layer was washed with water (×1), brine (×1), and dried over Na₂SO₄. The solids were filtered off, and the volatiles were removed via rotary evaporation. The residue was dissolved in DCM. Silica gel (5 g) was added, and the volatiles were removed via rotary evaporation. Purification via flash chromatography on silica gel eluting with 0-60% DCM in hexanes yielded 0.2002 g (0.58 mmol, 65% yield) of 169 as a white solid. Mass spectrum (ESI⁺): m/z=345 [M+1]observed.

169 was converted to 170 in a manner similar to that described above for 164. Mass spectrum (ESI⁺): m/z=315 [M+1]observed.

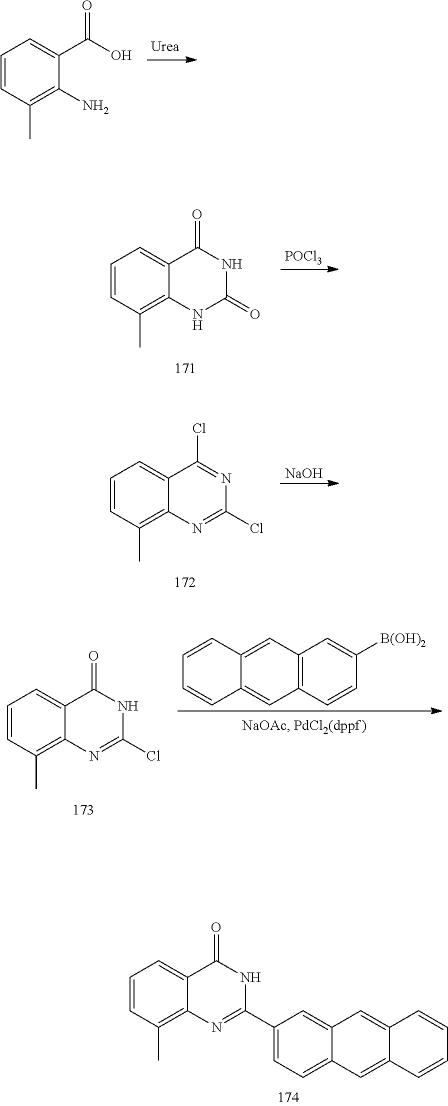

A 100 mL pear-shaped flask with stir bar was charged with 2-amino-3-methylbenzoic acid (2.0 g, 13.2 mmol, 1 eq) and urea (7.94 g, 132 mmol, 10 eq). The flask was evacuated and back-filled with Ar (×3). The resulting stirred mixture was heated to 150° C. overnight. After cooling to 100° C. water (40 mL) was added. The resulting hot mixture was filtered to collect the solids. The filter cake was triturated with MeOH (×3) and dried under vacuum. 2.82 g (16 mmol, 120% yield) of crude 171 was isolated as a pale solid. The crude was used in the next step without further purification.

A 50 mL round bottom flask with a reflux condenser and stir bar was charged with 171 (1.0 g, 5.7 mmol, 1 eq). The flask was evacuated and back-filled with Ar (×3). POCl₃ (2.6 mL, 4.35 g, 28.3 mmol, 5 eq) was added followed by DIPEA (2.0 mL, 1.47 g, 11.3 mmol, 2 eq). The resulting mixture was heated to reflux with stirring. After 5 h the reaction was cooled to room temperature and poured onto ice with vigorous stirring. The resulting suspension was cooled to 0° C. and adjusted to pH=7-8 with NH₄OH (aq, concentrated). The solids were collected via vacuum filtration. The solids were suspended in DCM and plug filtered through silica gel, eluting with DCM. The filtrate was concentrated to dryness via rotary evaporation yielding 0.467 g (2.19 mmol, 38% yield) of 172 as a white solid. Mass spectrum (ESI⁺): m/z=213 [M+1]observed.

A 100 mL round bottom flask with stir bar was charged with 172 (0.367 g, 1.72 mmol, 1 eq). THF (8 mL) was added. The resulting stirred solution was treated with 1N NaOH (4 mL, 4 mmol, 2.3 eq). After 2 h the volatiles were removed via rotary evaporation. The resulting mixture was adjusted to pH≈7 with 1N HCl. The solids were collected via vacuum filtration and air dried. 0.282 g (1.45 mmol, 84% yield) of 173 was isolated as a white solid.

Mass spectrum (ESI⁺): m/z=195 [M+1]observed.

A 20 mL, screw-top vial was charged with 173 (0.109 g, 0.56 mmol, 1 eq), anthracen-2-ylboronic acid (Ark Pharm, 0.1866 g, 0.84 mmol, 1.5 eq), NaOAc (0.2068 g, 2.5 mmol, 4.5 eq) and a stir bar. 1,4-dioxane (5 mL) and water (2 mL) were added. The vial was sealed with a septum, and the stirred mixture was sparged with Ar (10 min). PdCl₂(dppf) (0.0826 g, 0.11 mmol, 0.2 eq) was added. The vial was sealed with a lid, and the reaction was heated to 90° C. overnight. After cooling to room temperature the reaction was diluted with water/hexanes, and the solids were collected via vacuum filtration. The filter cake was triturated with 10% EtOAc in hexanes (×1) and 10% DCM in hexanes (×1). After drying 0.267 g (0.79 mmol, 142% yield) of impure 174 was isolated. The material was used without purification.

Mass spectrum (ESI⁺): m/z=337 [M+1]observed.

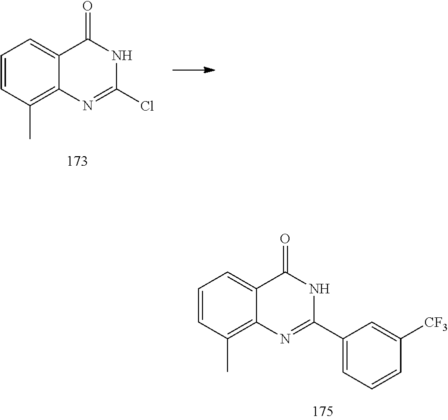 [SMILES: Cc1cccc2c(=O)[nH]c(Cl)nc12 ; Cc1cccc2c(=O)[nH]c(-c3cccc(C(F)(F)F)c3)nc12]

173 was converted to 175 in a manner similar to that described above for 174 using 3-trifluoromethylphenylboronic acid (Combi-Blocks).

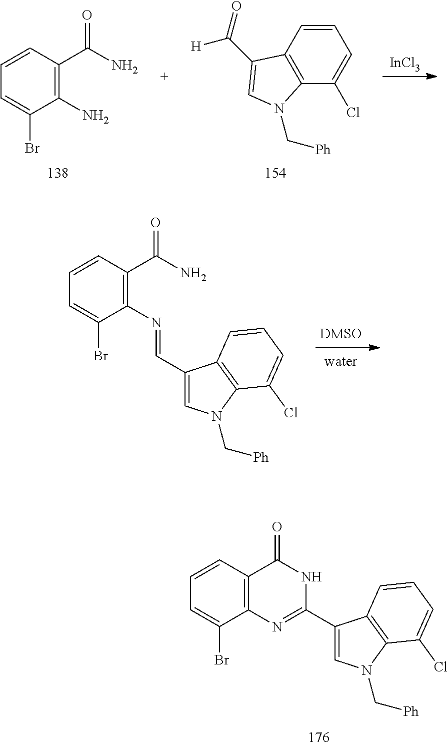

A 25 mL round bottom flask was charged with 138 (0.0797 g, 0.37 mmol, 1 eq), 154 (0.100 g, 0.37 mmol, 1 eq) and a stir bar. The vial was evacuated and back-filled with Ar (×3). Anhydrous CH₃CN (5 mL) was added. After 5 min the stirred mixture was treated with InCl₃ (0.025 g, 0.11 mmol, ≈30 mol %). The reaction was heated to 40° C. overnight. LCMS indicated the presence of the intermediate imine, but no starting materials or product. Mass spectrum (ESI⁺): m/z=466/468 [M+1]observed. The reaction was cooled to room temperature, and the solids were collected via vacuum filtration. After drying the solids were transferred to a 20 mL screw-top vial along with the stir bar. DMSO (3 mL) and water (0.3 mL) were added. The resulting mixture was heated to 120° C. overnight. After cooling to room temperature the reaction was diluted with water. The solids were collected via vacuum filtration. 0.113 g of crude 176 was collected and used without purification. Mass spectrum (ESI⁺): m/z=464/466 [M+1]observed.

The following quinazolinones were synthesized in manner similar to that described for 176 above. Formation of the imine was performed at room temperature to 40° C. Conversion to the quinazolinone was carried out at temperatures of 80 to 150° C.

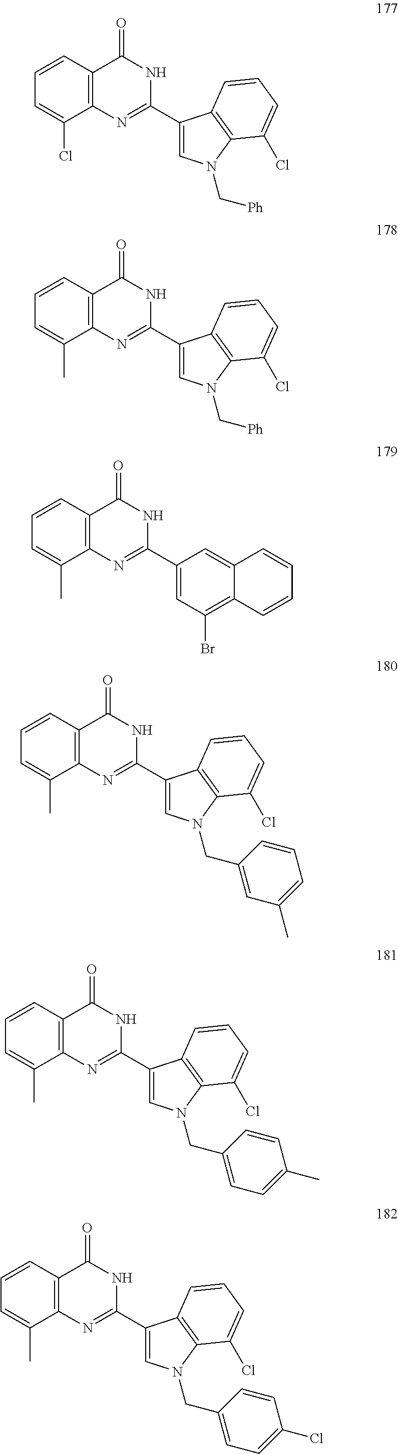 [SMILES: O=c1[nH]c(-c2cn(Cc3ccccc3)c3c(Cl)cccc23)nc2c(Cl)cccc12 ; Cc1cccc2c(=O)[nH]c(-c3cn(Cc4ccccc4)c4c(Cl)cccc34)nc12 ; Cc1cccc2c(=O)[nH]c(-c3cc(Br)c4ccccc4c3)nc12 ; Cc1cccc(Cn2cc(-c3nc4c(C)cccc4c(=O)[nH]3)c3cccc(Cl)c32)c1 ; Cc1ccc(Cn2cc(-c3nc4c(C)cccc4c(=O)[nH]3)c3cccc(Cl)c32)cc1 ; Cc1cccc2c(=O)[nH]c(-c3cn(Cc4ccc(Cl)cc4)c4c(Cl)cccc34)nc12]

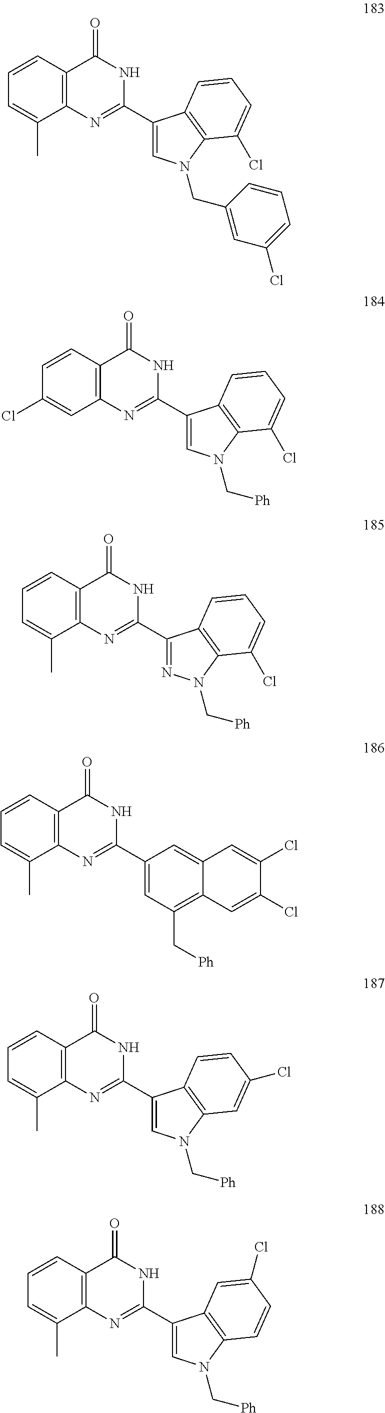 [SMILES: Cc1cccc2c(=O)[nH]c(-c3cn(Cc4cccc(Cl)c4)c4c(Cl)cccc34)nc12 ; O=c1[nH]c(-c2cn(Cc3ccccc3)c3c(Cl)cccc23)nc2cc(Cl)ccc12 ; Cc1cccc2c(=O)[nH]c(-c3nn(Cc4ccccc4)c4c(Cl)cccc34)nc12 ; Cc1cccc2c(=O)[nH]c(-c3cc(Cc4ccccc4)c4cc(Cl)c(Cl)cc4c3)nc12 ; Cc1cccc2c(=O)[nH]c(-c3cn(Cc4ccccc4)c4cc(Cl)ccc34)nc12 ; Cc1cccc2c(=O)[nH]c(-c3cn(Cc4ccccc4)c4ccc(Cl)cc34)nc12]

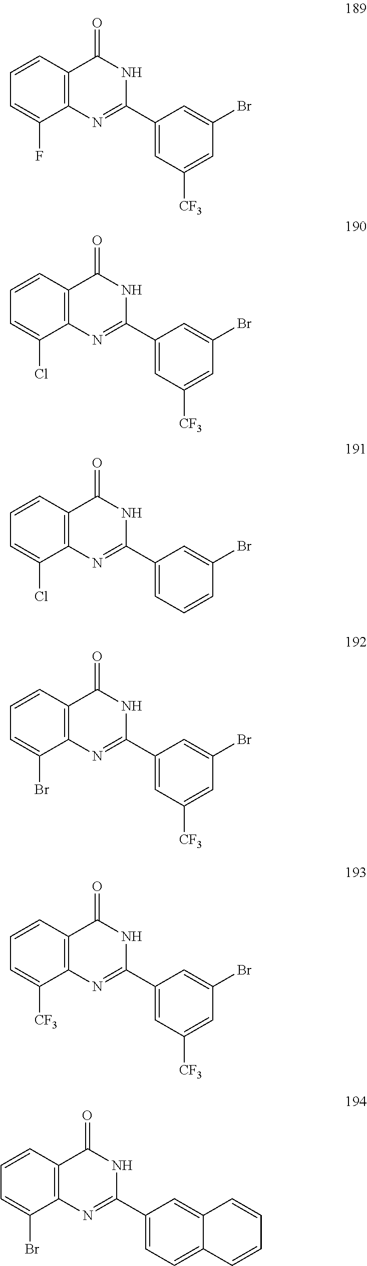 [SMILES: O=c1[nH]c(-c2cc(Br)cc(C(F)(F)F)c2)nc2c(F)cccc12 ; O=c1[nH]c(-c2cc(Br)cc(C(F)(F)F)c2)nc2c(Cl)cccc12 ; O=c1[nH]c(-c2cccc(Br)c2)nc2c(Cl)cccc12 ; O=c1[nH]c(-c2cc(Br)cc(C(F)(F)F)c2)nc2c(Br)cccc12 ; O=c1[nH]c(-c2cc(Br)cc(C(F)(F)F)c2)nc2c(C(F)(F)F)cccc12 ; O=c1[nH]c(-c2ccc3ccccc3c2)nc2c(Br)cccc12]

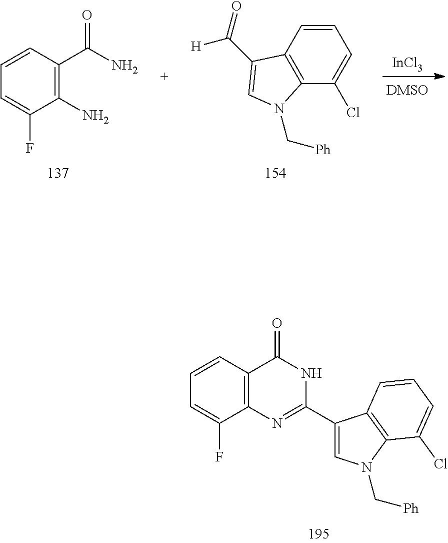

A 20 mL screw-top vial was charged with 137 (0.0571 g, 0.37 mmol, 1 eq), 154 (0.100 g, 0.37 mmol, 1 eq) and a stir bar. The vial was evacuated and back-filled with Ar (×3). Anhydrous DMSO (2 mL) was added. The resulting stirred solution was treated with InCl₃ (0.0246 g, 0.1 mmol, 0.27 eq). The vial was closed with the lid, and the reaction was heated to 100° C. overnight. LCMS indicated the reaction was not complete. The reaction was heated to 125° C. for 5 h. After cooling to room temperature the reaction was diluted with water. The solids were collected via vacuum filtration yielding 0.090 g (0.22 mmol, 60% yield) of crude 195 as a solid. The material was carried forward with purification. Mass spectrum (ESI⁺): m/z=404 [M+1]observed.

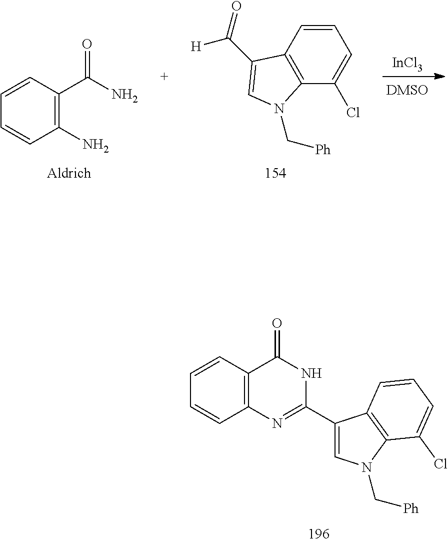

Anthranilamide was converted to 196 in a manner similar to that described above for 195.

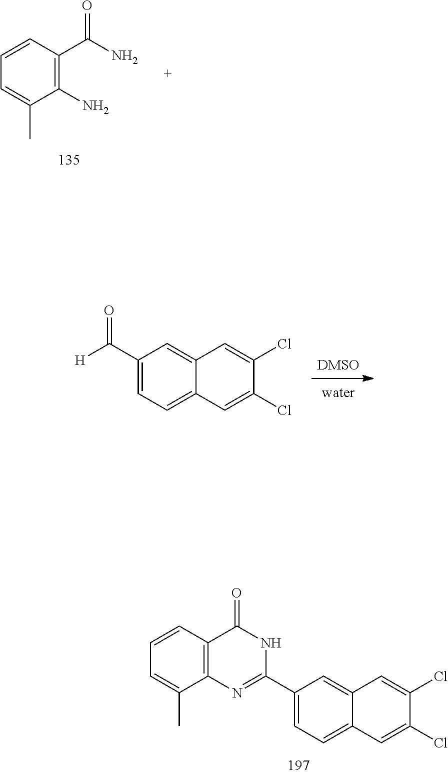 [SMILES: Cc1cccc(C(N)=O)c1N ; O=Cc1ccc2cc(Cl)c(Cl)cc2c1 ; Cc1cccc2c(=O)[nH]c(-c3ccc4cc(Cl)c(Cl)cc4c3)nc12]

A 20 mL screw-top vial was charged with 135 (0.0451 g, 0.30 mmol, 1 eq), 164 (0.0676 g, 0.3 mmol, 1 eq) and a stir bar. DMSO (3 mL) was added along with a few drops of water. The vial was loosely sealed with the lid, and the reaction was heated to 150° C. overnight. After cooling to room temperature the reaction was diluted with water, and the solids were collected via vacuum filtration. The filter cake was triturated with 10% DCM in hexanes and dried yielding 0.0493 g (0.14 mmol, 46% yield) of 197 as a tan solid. Mass spectrum (ESI⁺): m/z=355 [M+1]observed. The crude was used without purification.

The following quinazolinones were synthesized in manner similar to that described for 197 above at temperatures of 100 to 150° C.

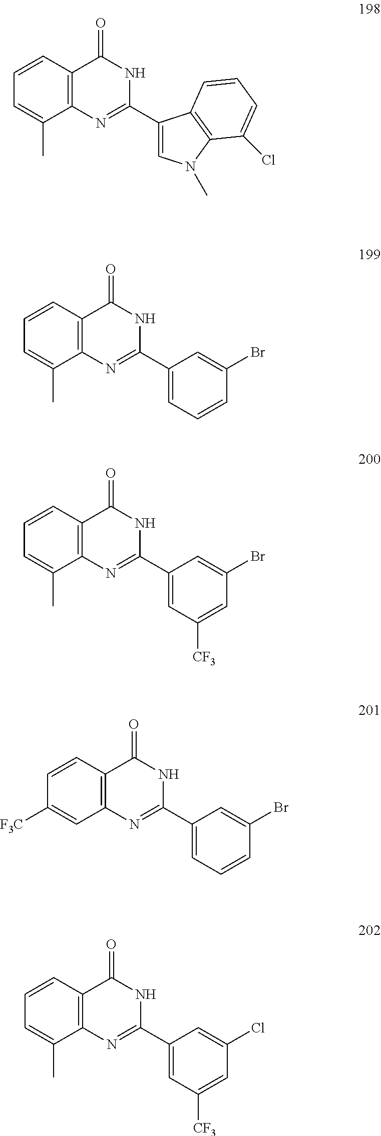 [SMILES: Cc1cccc2c(=O)[nH]c(-c3cn(C)c4c(Cl)cccc34)nc12 ; Cc1cccc2c(=O)[nH]c(-c3cccc(Br)c3)nc12 ; Cc1cccc2c(=O)[nH]c(-c3cc(Br)cc(C(F)(F)F)c3)nc12 ; O=c1[nH]c(-c2cccc(Br)c2)nc2cc(C(F)(F)F)ccc12 ; Cc1cccc2c(=O)[nH]c(-c3cc(Cl)cc(C(F)(F)F)c3)nc12]

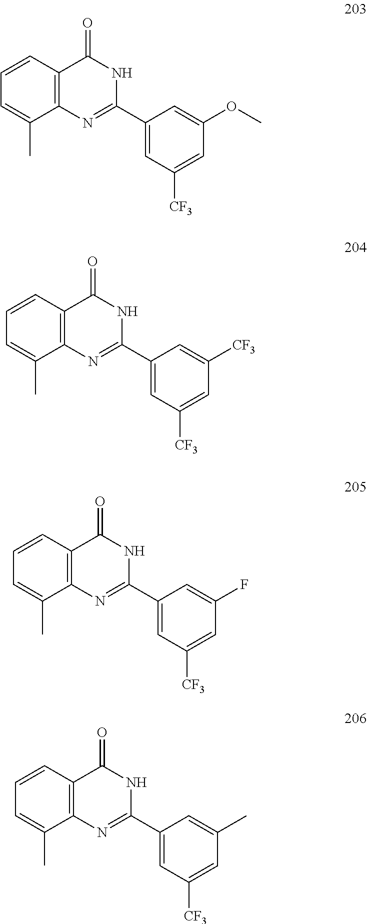 [SMILES: COc1cc(-c2nc3c(C)cccc3c(=O)[nH]2)cc(C(F)(F)F)c1 ; Cc1cccc2c(=O)[nH]c(-c3cc(C(F)(F)F)cc(C(F)(F)F)c3)nc12 ; Cc1cccc2c(=O)[nH]c(-c3cc(F)cc(C(F)(F)F)c3)nc12 ; Cc1cc(-c2nc3c(C)cccc3c(=O)[nH]2)cc(C(F)(F)F)c1]

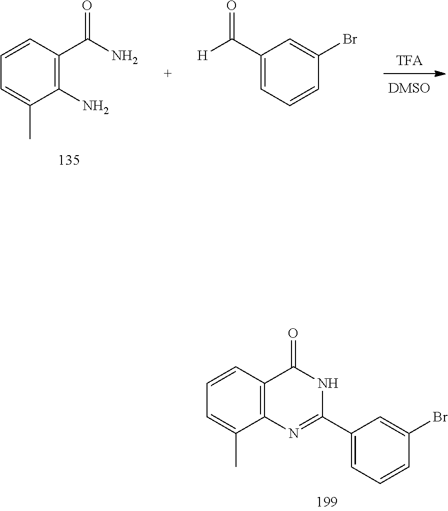 [SMILES: Cc1cccc(C(N)=O)c1N ; O=Cc1cccc(Br)c1 ; Cc1cccc2c(=O)[nH]c(-c3cccc(Br)c3)nc12]

A 100 mL round bottom flask was charged with 135 (0.400 g, 2.7 mmol, 1 eq) and a stir bar. DMSO (30 mL) was added followed by 3-bromobenzaldehyde (0.31 mL, 0.498 g, 2.7 mmol, 1 eq). The resulting stirred solution was treated with TFA (20 μL, 0.0303 g, 0.27 mmol, 0.1 eq). After 15 min the reaction was heated to 125° C. overnight. After cooling to room temperature the mixture was diluted with water. The solids were collected via vacuum filtration. The filter cake was triturated with water (×3) and 10% DCM in hexanes (×3). After drying 0.731 g (2.3 mmol, 87% yield) of 199 was isolated as an off-white solid. Mass spectrum (ESI⁺): m/z=315/317 [M+1]observed.

The following quinazolinones were synthesized in manner similar at temperatures ranging from 80 to 150° C.

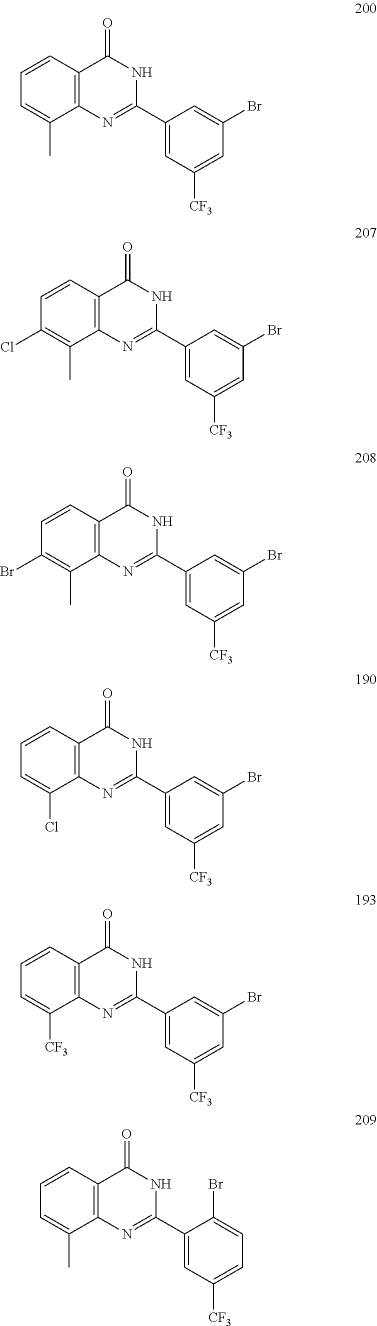 [SMILES: Cc1cccc2c(=O)[nH]c(-c3cc(Br)cc(C(F)(F)F)c3)nc12 ; Cc1c(Cl)ccc2c(=O)[nH]c(-c3cc(Br)cc(C(F)(F)F)c3)nc12 ; Cc1c(Br)ccc2c(=O)[nH]c(-c3cc(Br)cc(C(F)(F)F)c3)nc12 ; O=c1[nH]c(-c2cc(Br)cc(C(F)(F)F)c2)nc2c(Cl)cccc12 ; O=c1[nH]c(-c2cc(Br)cc(C(F)(F)F)c2)nc2c(C(F)(F)F)cccc12 ; Cc1cccc2c(=O)[nH]c(-c3cc(C(F)(F)F)ccc3Br)nc12]

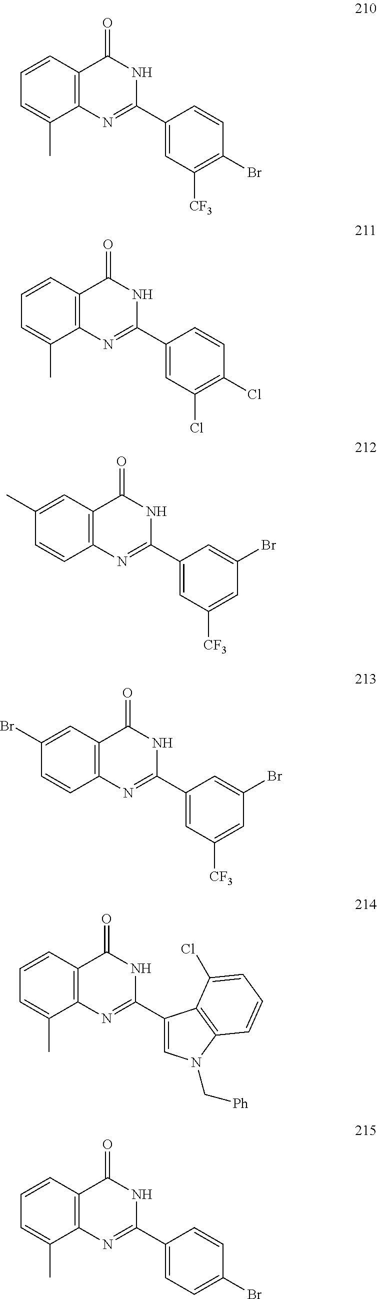 [SMILES: Cc1cccc2c(=O)[nH]c(-c3ccc(Br)c(C(F)(F)F)c3)nc12 ; Cc1cccc2c(=O)[nH]c(-c3ccc(Cl)c(Cl)c3)nc12 ; Cc1ccc2nc(-c3cc(Br)cc(C(F)(F)F)c3)[nH]c(=O)c2c1 ; O=c1[nH]c(-c2cc(Br)cc(C(F)(F)F)c2)nc2ccc(Br)cc12 ; Cc1cccc2c(=O)[nH]c(-c3cn(Cc4ccccc4)c4cccc(Cl)c34)nc12 ; Cc1cccc2c(=O)[nH]c(-c3ccc(Br)cc3)nc12]

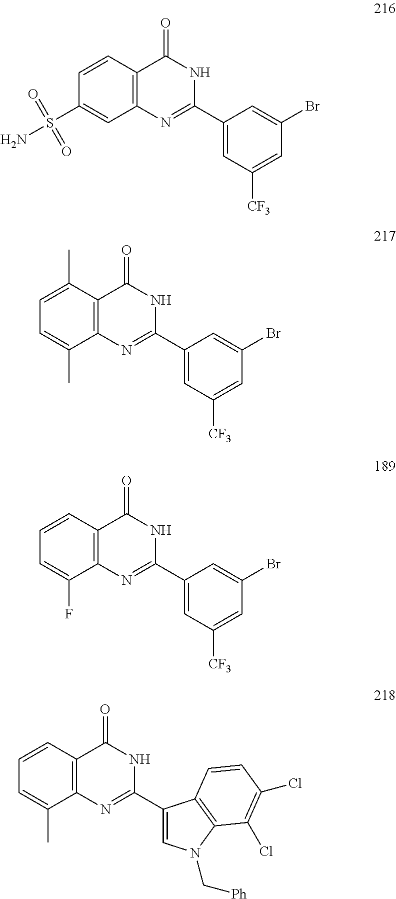 [SMILES: NS(=O)(=O)c1ccc2c(=O)[nH]c(-c3cc(Br)cc(C(F)(F)F)c3)nc2c1 ; Cc1ccc(C)c2c(=O)[nH]c(-c3cc(Br)cc(C(F)(F)F)c3)nc12 ; O=c1[nH]c(-c2cc(Br)cc(C(F)(F)F)c2)nc2c(F)cccc12 ; Cc1cccc2c(=O)[nH]c(-c3cn(Cc4ccccc4)c4c(Cl)c(Cl)ccc34)nc12]

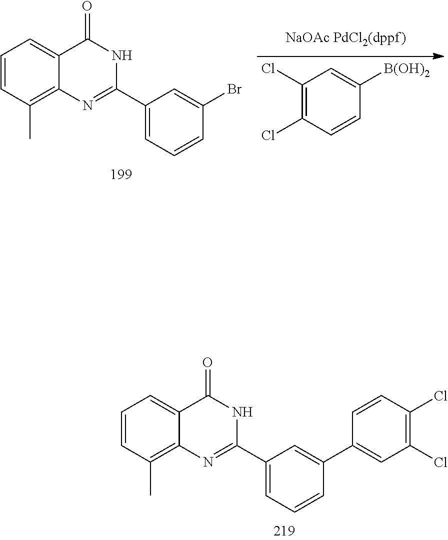

A 20 mL screw-top vial was charged with 199 (0.070 g, 0.22 mmol, 1 eq), 3,4-dichlorophenylboronic acid (Combi-Blocks, 0.0646 g, 0.33 mmol, 1.5 eq), NaOAc (0.0820 g, 1.0 mmol, 4.5 eq), and a stir bar. 1,4-dioxane/water (10:1, 5 mL) was added. The vial was sealed with a septum. The resulting stirred mixture was sparged with Ar (10 min). PdCl₂(dppf) (0.0325 g, 0.04 mmol, 0.2 eq) was added. The vial was closed with the lid. The reaction was heated to 90° C. overnight. After cooling to room temperature the reaction was diluted with water. The solids were collected via vacuum filtration. The filter cake was triturated with water (×3), 10% DCM in hexanes (×3), and hexanes (×1). After drying 0.0835 g (0.22 mmol, 99% yield) of 219 was isolated as an ash-colored solid. Mass spectrum (ESI⁺): m/z=381 [M+1]observed.

The following quinazolinones were synthesized in manner similar to that described for 219 above.

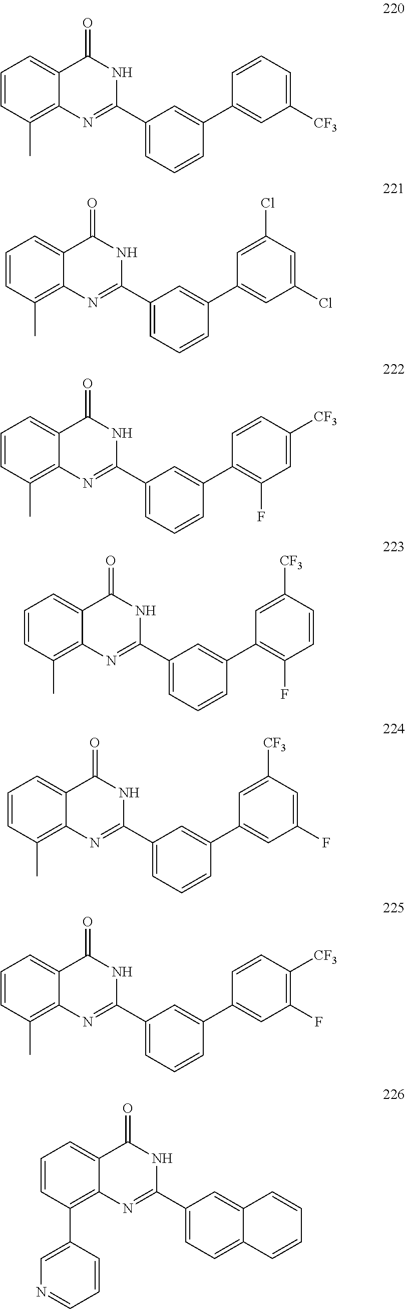

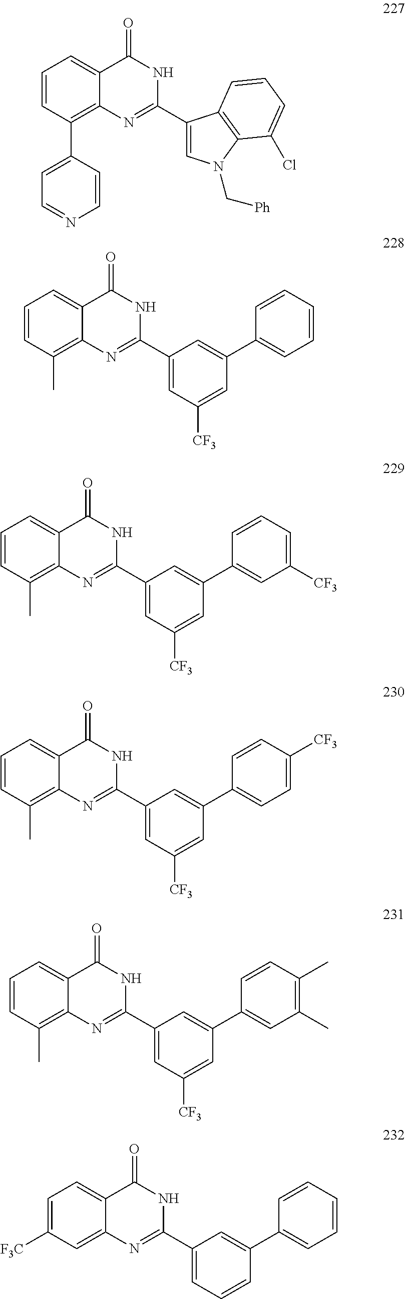 [SMILES: O=c1[nH]c(-c2cn(Cc3ccccc3)c3c(Cl)cccc23)nc2c(-c3ccncc3)cccc12 ; Cc1cccc2c(=O)[nH]c(-c3cc(-c4ccccc4)cc(C(F)(F)F)c3)nc12 ; Cc1cccc2c(=O)[nH]c(-c3cc(-c4cccc(C(F)(F)F)c4)cc(C(F)(F)F)c3)nc12 ; Cc1cccc2c(=O)[nH]c(-c3cc(-c4ccc(C(F)(F)F)cc4)cc(C(F)(F)F)c3)nc12 ; Cc1ccc(-c2cc(-c3nc4c(C)cccc4c(=O)[nH]3)cc(C(F)(F)F)c2)cc1C ; O=c1[nH]c(-c2cccc(-c3ccccc3)c2)nc2cc(C(F)(F)F)ccc12]

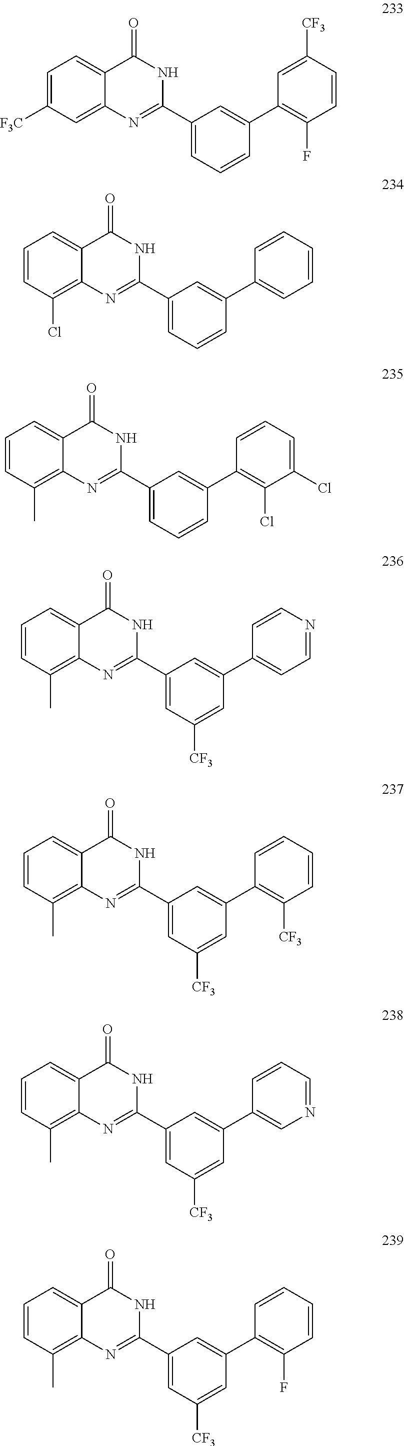

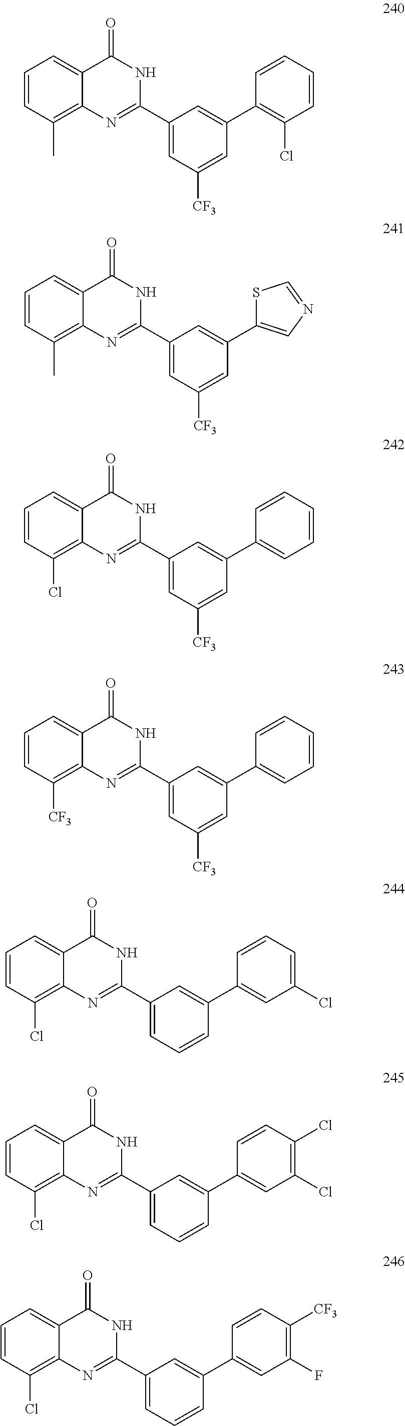

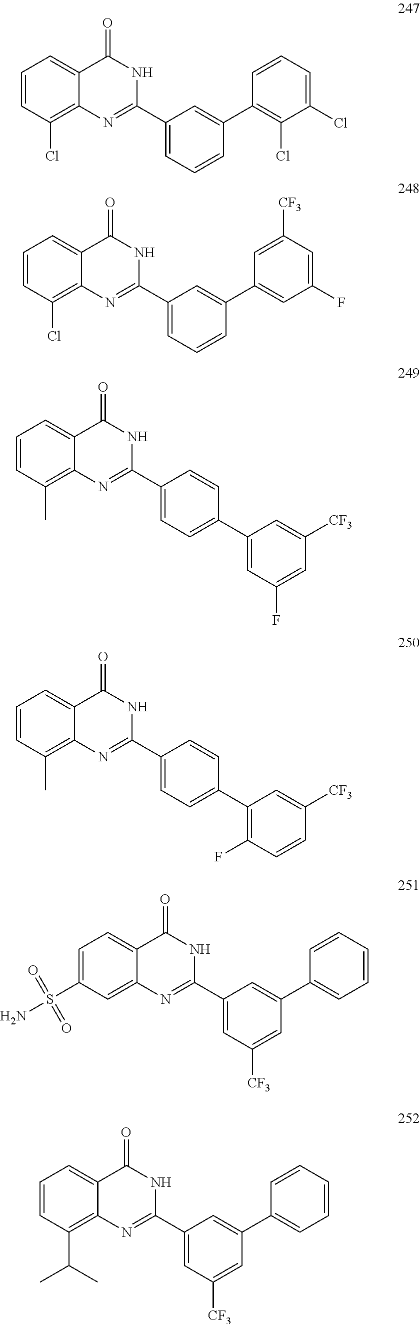

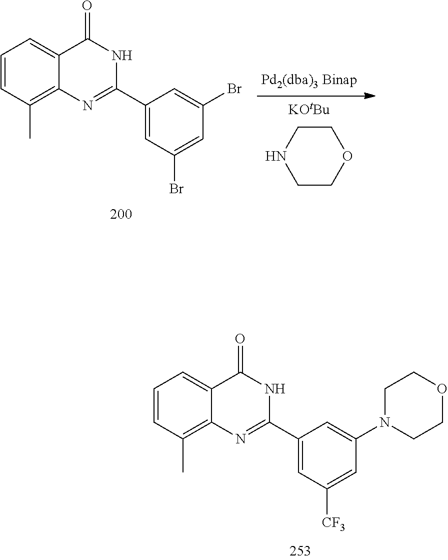 [SMILES: Cc1cccc2c(=O)[nH]c(-c3cc(Br)cc(Br)c3)nc12 ; C1COCCN1 ; Cc1cccc2c(=O)[nH]c(-c3cc(N4CCOCC4)cc(C(F)(F)F)c3)nc12 ; "KOtBu"]

A 20 mL screw-top vial was charged with 200 (0.100 g, 0.26 mmol, 1 eq), KO^{t}Bu (0.0439 g, 0.39 mmol, 1.5 eq), Pd₂(dba)₃ (0.0239 g, 0.026 mmol, 10 mol %), binap (0.0325 g, 0.052 mmol, 20 mol %), and a stir bar. The vial was evacuated and back-filled with Ar (×3). A solution of morpholine (57 μL, 0.0568 g, 0.65 mmol, 2.5 eq) in anhydrous 1,4-dioxane (3 mL) was sparged with Ar (10 min) and added to the vial. The vial was closed with the lid, and the reaction was heated to 100° C. overnight. LCMS indicated the presence of unreacted 200. The reaction was heated to 120° C. overnight. After cooling to room temperature the reaction was diluted with water/hexanes, and the solids were collected via vacuum filtration. Purification of the crude via flash chromatography eluting with 0-60% EtOAc in hexanes yielded 0.0616 g (0.16 mmol, 61% yield) of 253 as a tan solid. Mass spectrum (ESI⁺): m/z=390 [M+1]observed.

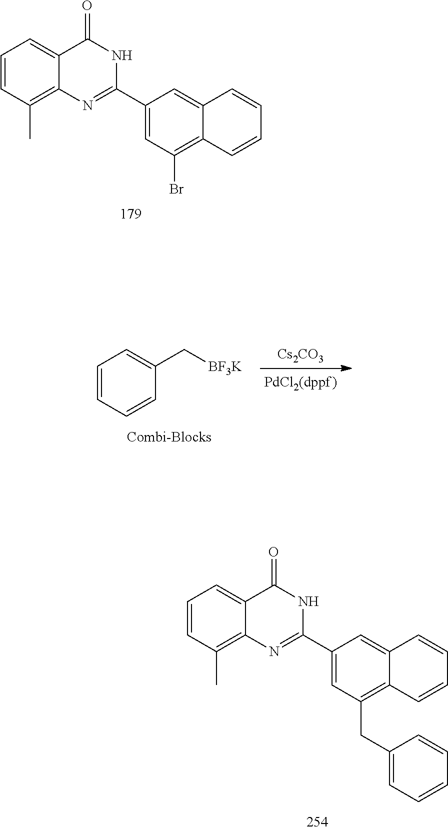

A 20 mL screw-top vial was charged with 179 (0.100 g, 0.27 mmol, 1 eq), potassium benzyltrifluoroborate (0.0678 g, 0.34 mmol, 1.25 eq), Cs₂CO₃ (0.2676 g, 0.82 mmol, 3 eq) and a stir bar. 1,4-dioxane/water (5:1, 5 mL) was added. The resulting stirred mixture was sparged with Ar (10 min). PdCl₂(dppf) (0.020 g, 0.027 mmol, 0.1 eq) was added. The vial was closed with the lid. The reaction was heated to 90° C. overnight. After cooling to room temperature the mixture was diluted with water/hexanes. The solids were collected via vacuum filtration. The filter cake was triturated with water (×1) and 10% DCM in hexanes (×1). After drying 0.0645 g (0.17 mmol, 63% yield) of 254 was isolated as a dark colored solid. Mass spectrum (ESI⁺): m/z=377 [M+1]observed.

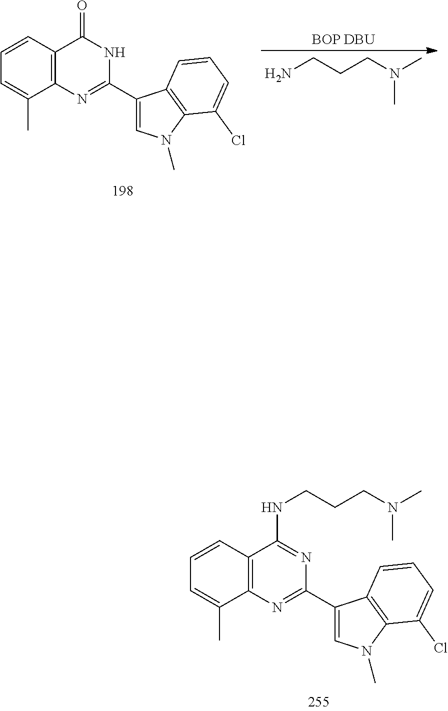

A 25 mL round bottom flask was charged with 198 (0.0464 g, 0.14 mmol, 1 eq), BOP (0.0824 g, 0.19 mmol, 1.3 eq), and a stir bar. The vial was evacuated and back-filled with Ar (×3). Anhydrous DMF (2 mL) was added with stirring. After 5 min DBU (32 μL, 0.033 g, 0.21 mmol, 1.5 eq) was added followed by 3-dimethylamino-1-propylamine (Aldrich, 27 μL, 0.022 g, 0.21 mmol, 1.5 eq) 15 min later. The reaction was stirred at room temperature for 40 min before heating to 55° C. After 3 h the heating was turned off, and the reaction was stirred overnight. The reaction was diluted with EtOAc/water, and the layers were separated. The organic layer was washed with water (×2), brine (×1), and dried over Na₂SO₄. The solids were filtered off, and the volatiles were removed via rotary evaporation. Purification via flash chromatography on silica gel eluting with 0-20% MeOH in DCM yielded 0.0028 g (0.007 mmol, 4.8% yield) of 255. ¹H NMR (CDCl₃, 300 MHz) δ 8.84 (d, J=6.9 Hz, 1H), 8.02 (s, 1H), 7.87 (broad s, 1H), 7.67 (d, J=7.8 Hz, 1H), 7.55 (d, J=6.3 Hz, 1H), 7.27-7.13 (m, 3H), 4.25 (s, 3H), 3.92 (broad s, 2H), 2.83-2.80 (m, 5H), 2.51 (s, 6H), 2.11-2.07 (m, 2H). Mass spectrum (ESI⁺): m/z=408 [M+1].

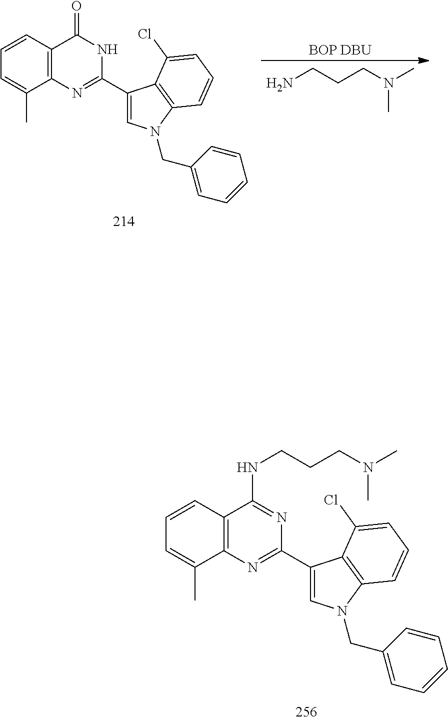

A 25 mL round bottom flask was charged with 214 (0.040 g, 0.1 mmol, 1 eq) and a stir bar. The flask was evacuated and back-filled with Ar (×3). Anhydrous DMF (4 mL) was added. The resulting stirred suspension was treated with DBU (22.8 μL, 0.0247 g, 0.16 mmol, 1.6 eq). After 5 min the resulting solution was treated with 3-dimethylamino-1-propylamine (25.2 μL, 0.0204 g, 0.20 mmol, 2 eq). BOP (0.0619 g, 0.14 mmol, 1.4 eq) was added 10 min later. The reaction was stirred overnight. The reaction was diluted with water/MTBE. After stirring for 10 min the mixture was filtered, rinsing with MTBE. The layers were separated. The organic layer was washed with water (×3), brine (×1), and dried over Na₂SO₄. The solids were filtered off and the volatiles were removed via rotary evaporation. Purification via flash chromatography on silica gel eluting with 0-20% solvent B (B=5% conc. NH₃(aq) in MeOH) in EtOAc yielded 0.0039 g (0.008 mmol, 8% yield) of 256. ¹H NMR (CDCl₃, 300 MHz) δ 8.21 (broad s, 1H), 7.81-7.80 (m, 1H), 7.54-7.52 (m, 2H), 7.32-7.18 (m, 8H), 7.11-7.08 (m, 1H), 5.35 (s, 2H), 3.86 (broad s, 2H), 2.74 (s, 3H), 2.64-2.61 (m, 2H), 2.40 (s, 6H), 2.07-2.04 (m, 2H). Mass spectrum (ESI⁺): m/z=484 [M+1].

The following quinazolines were synthesized from the appropriate quinazolinone in a manner similar to that described for either 255 or 256 above using either CH₃CN or DMF as solvent. The amines were purchased from the vendor indicated and used without purification.

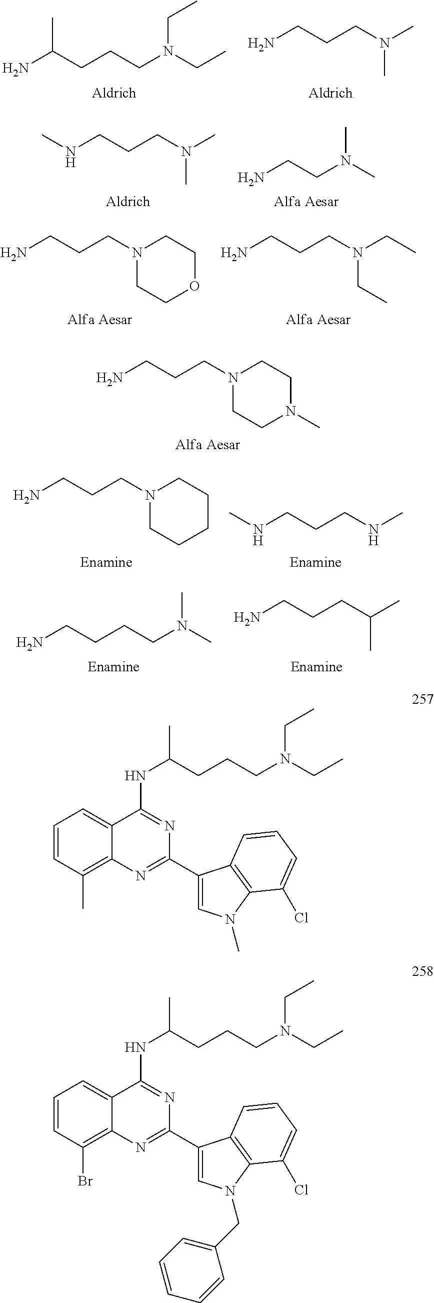

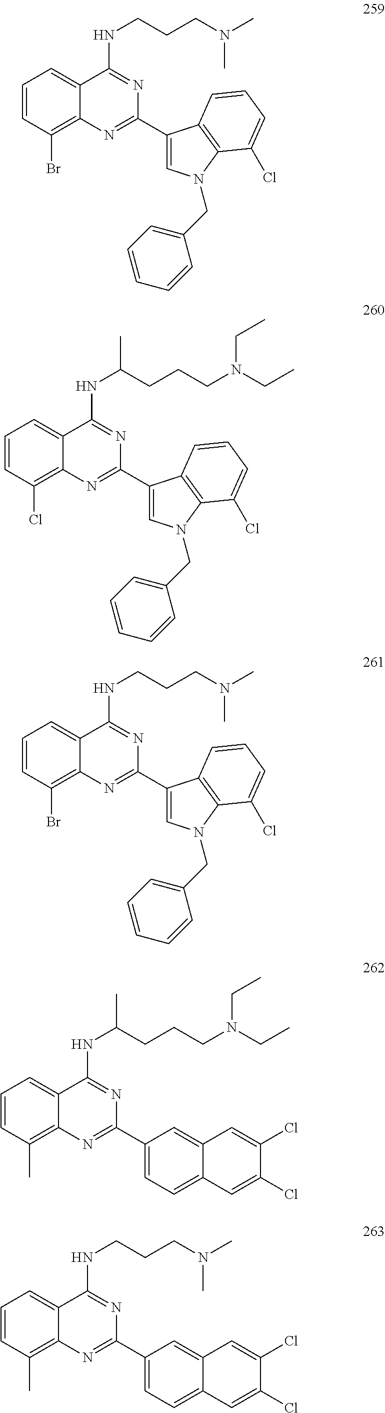

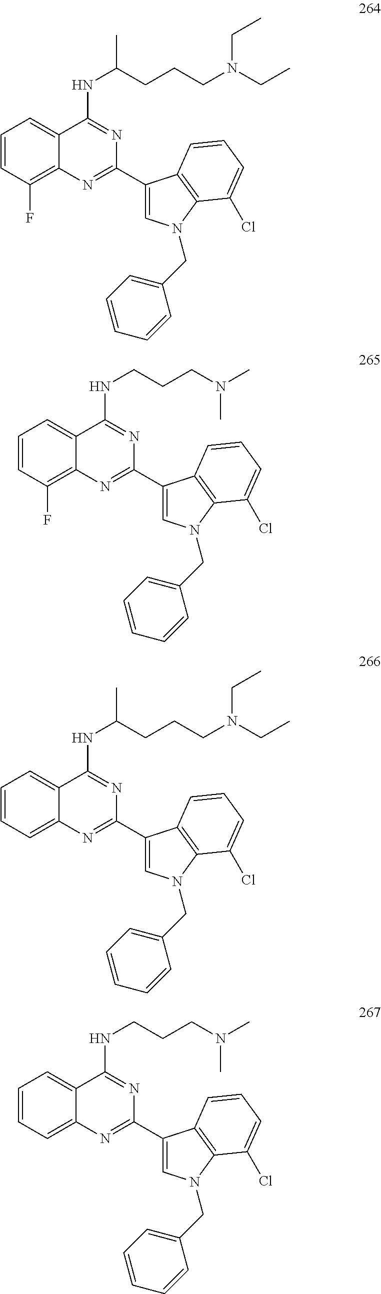

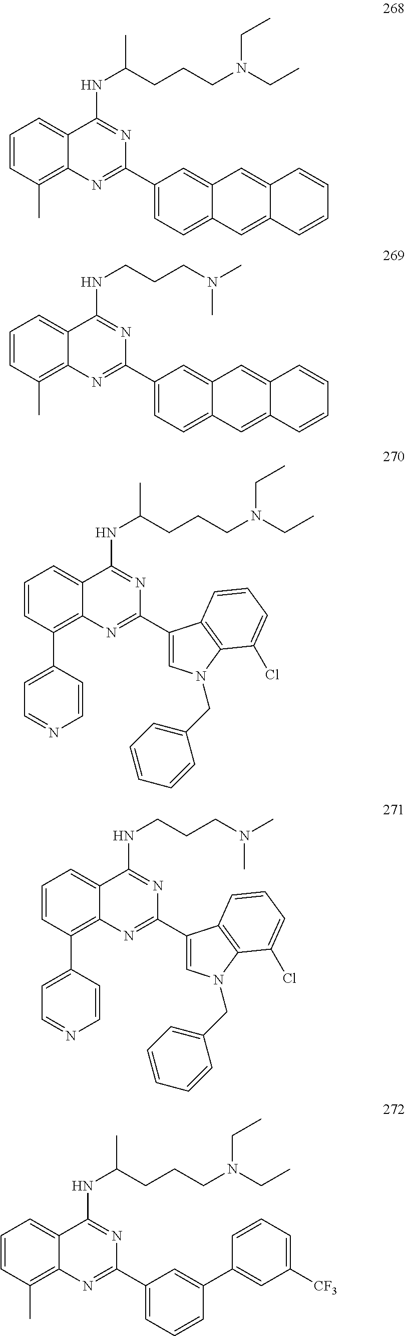

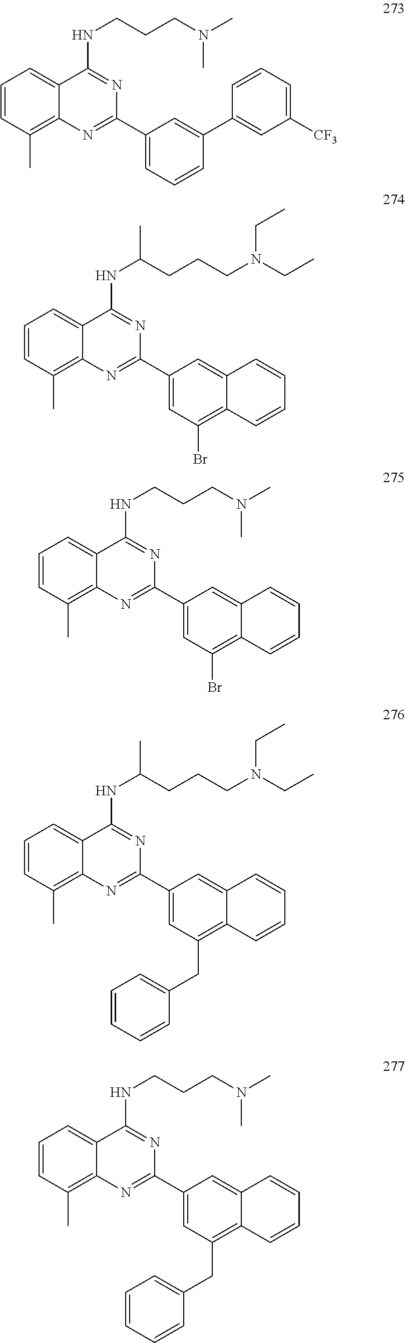

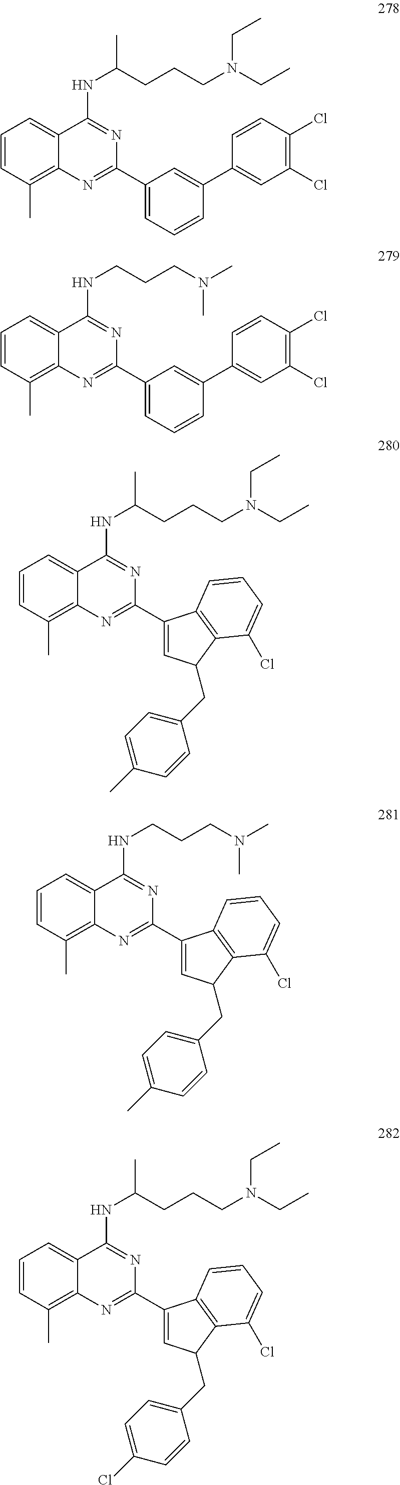

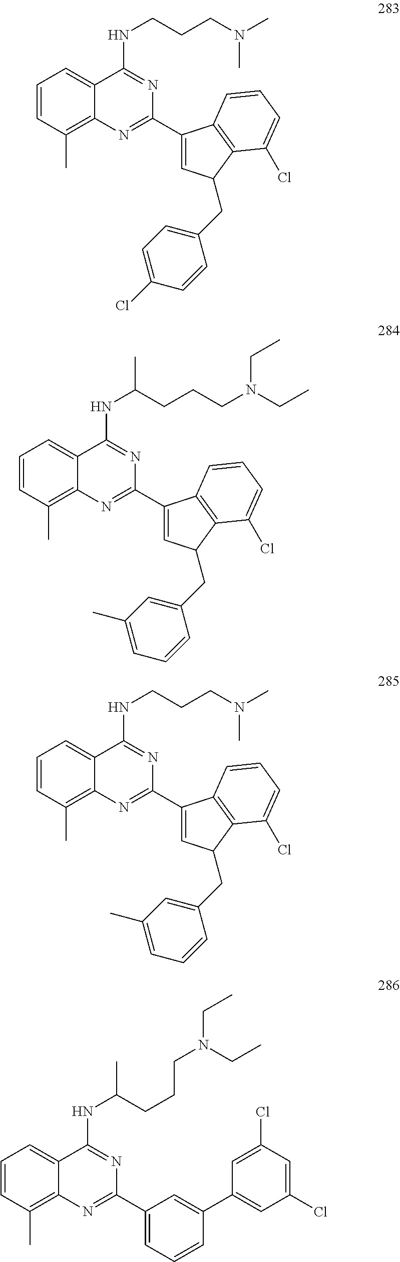

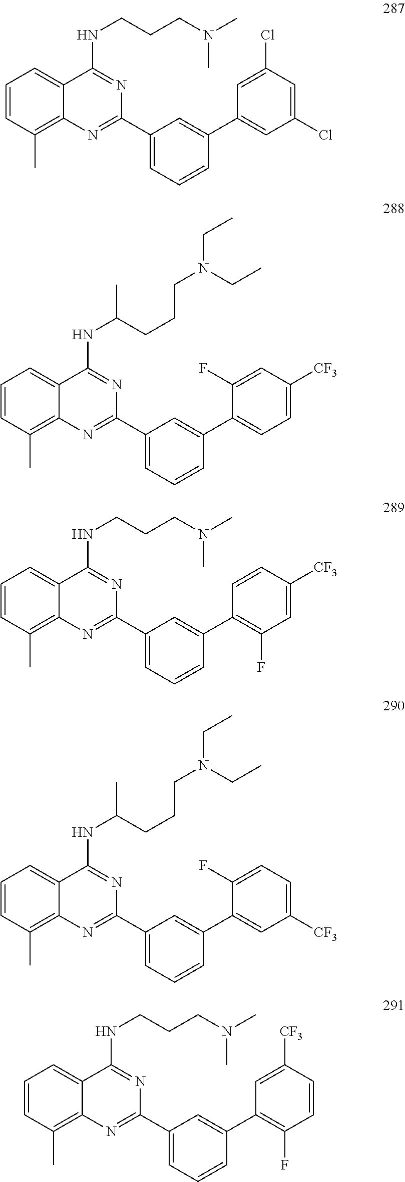

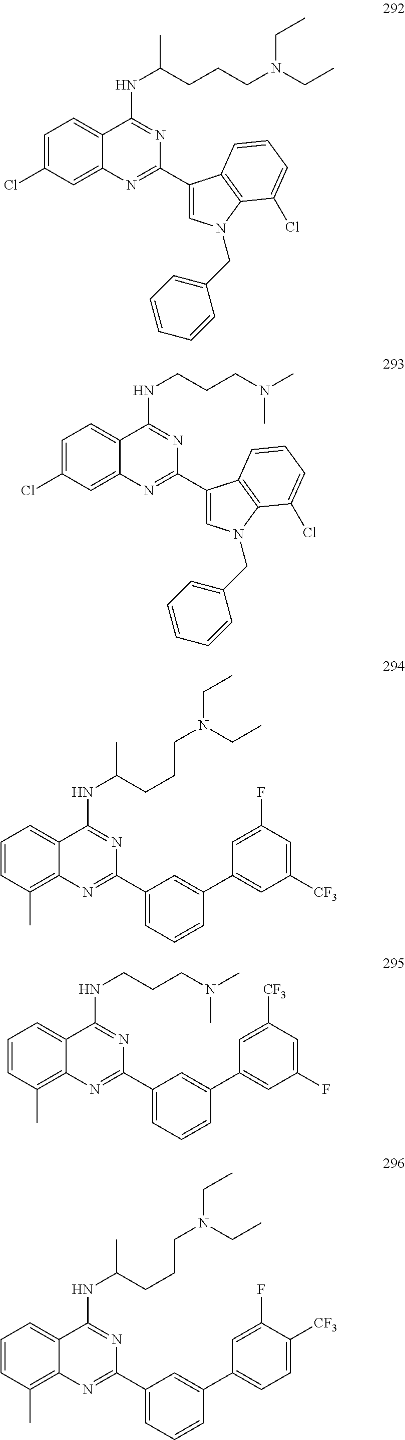

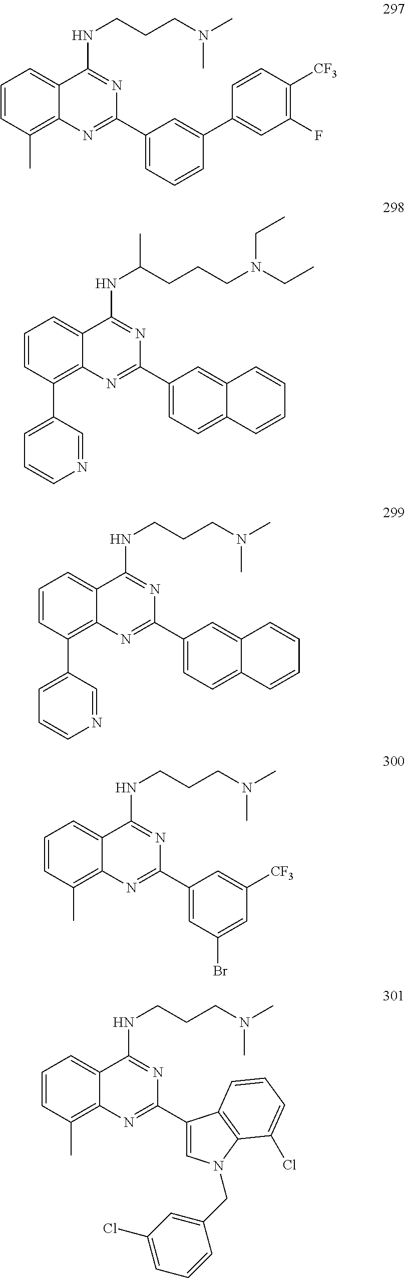

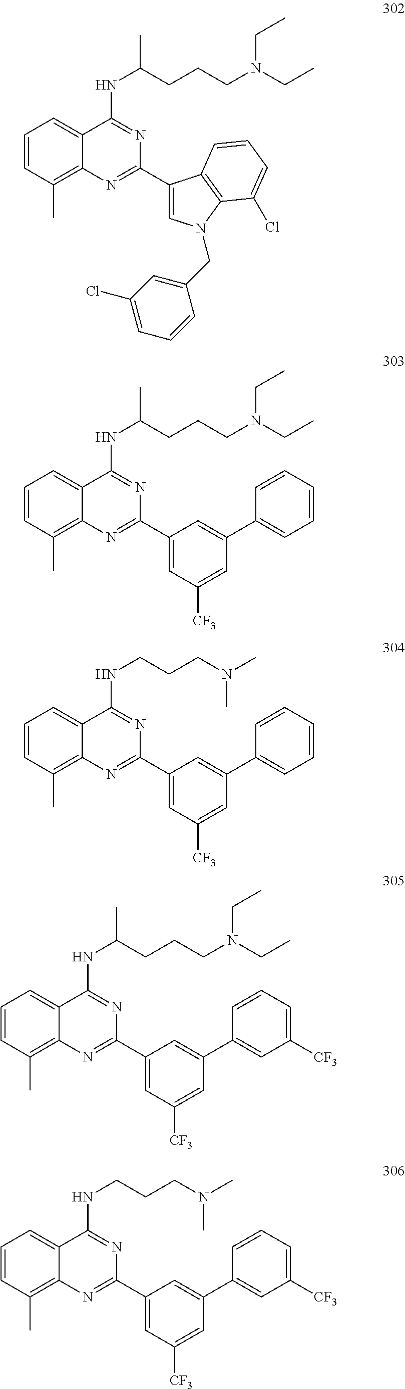

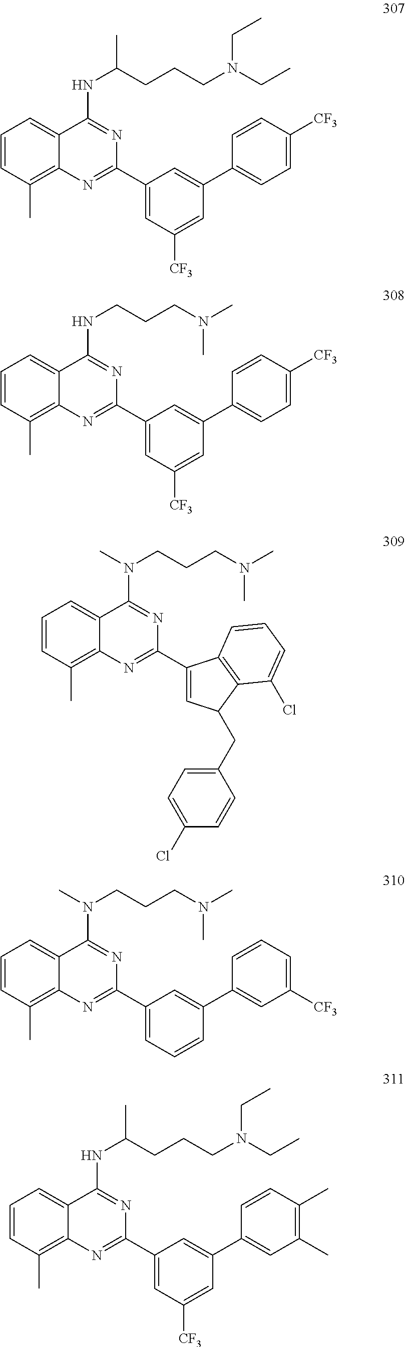

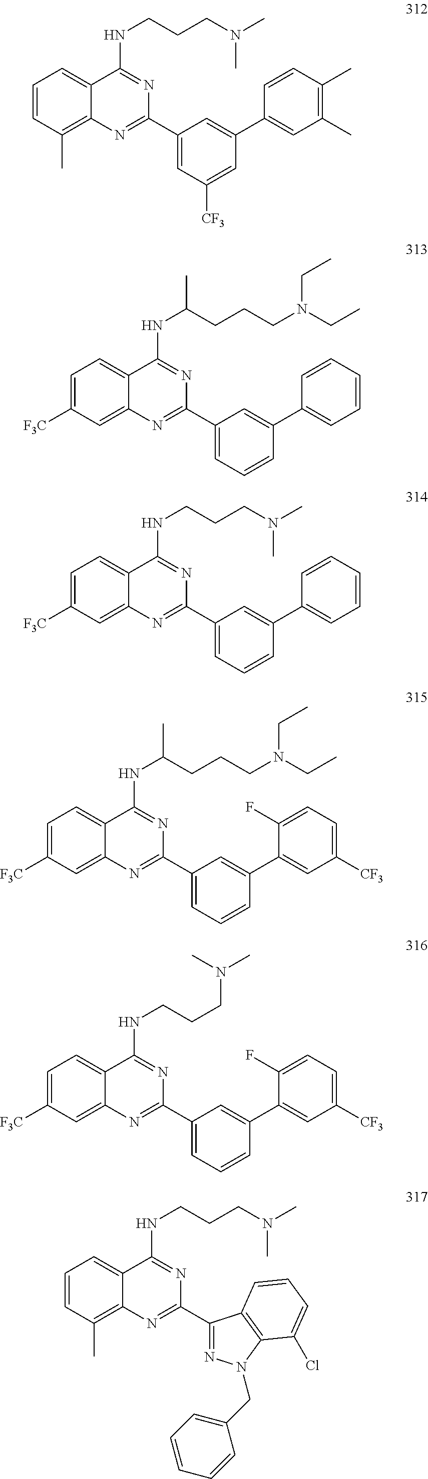

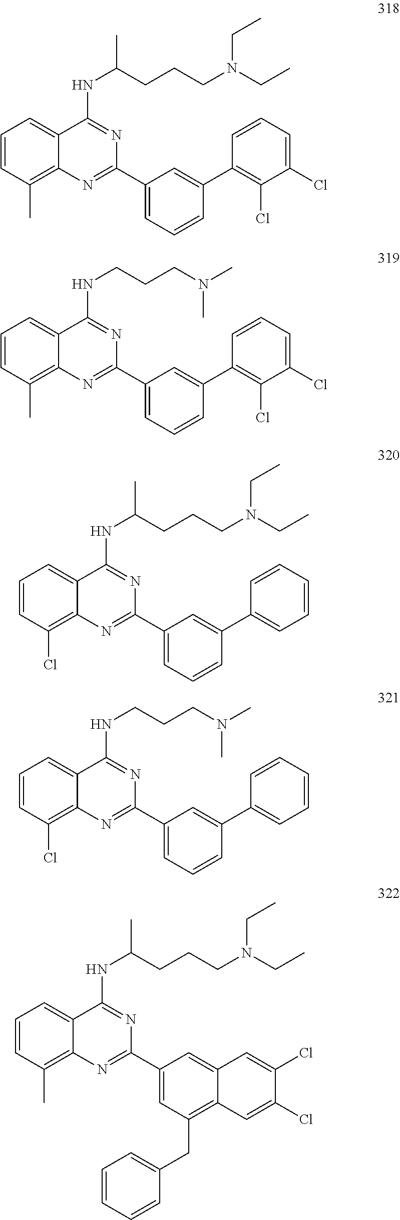

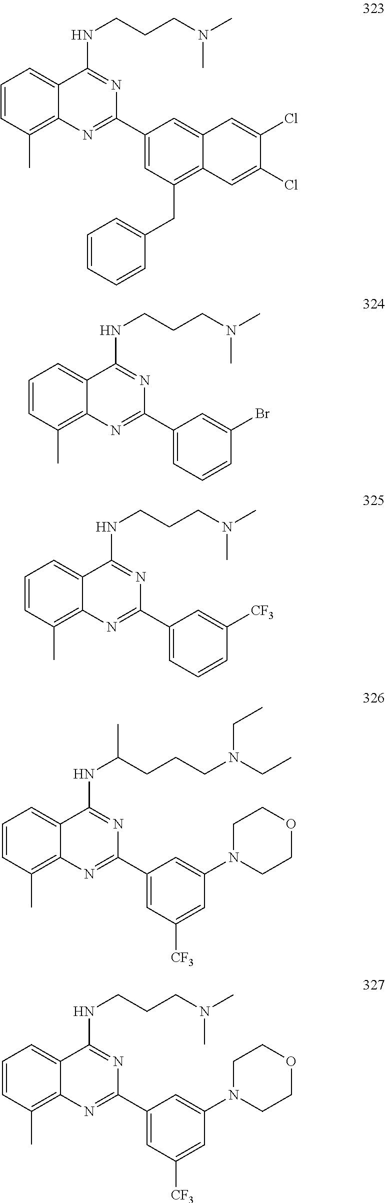

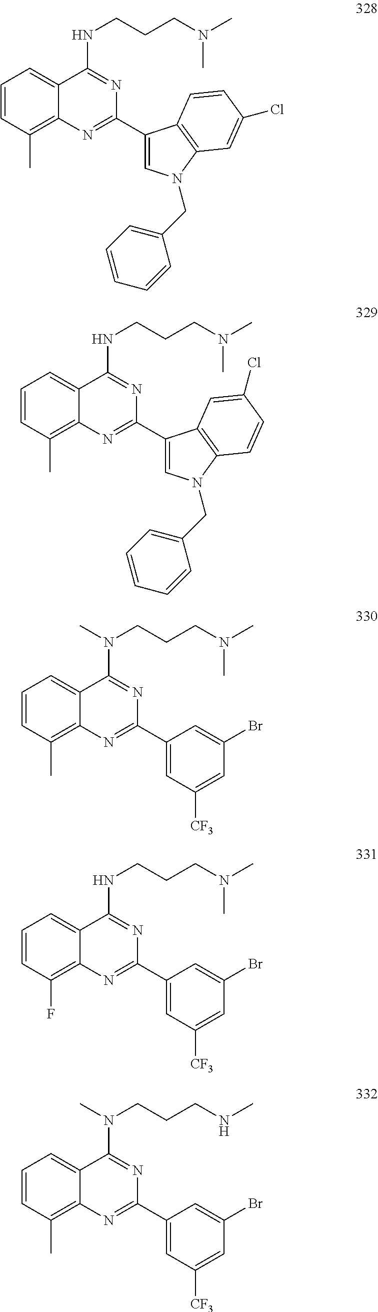

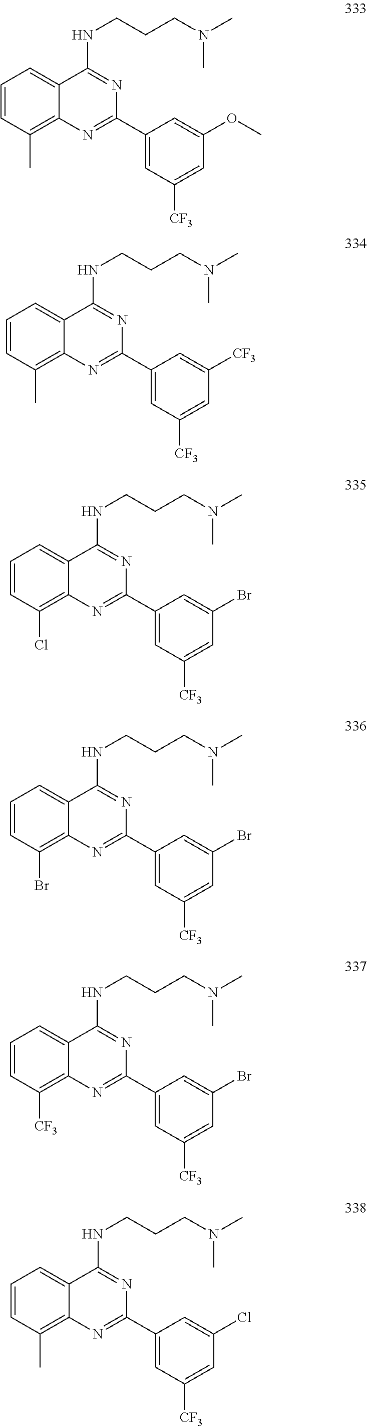

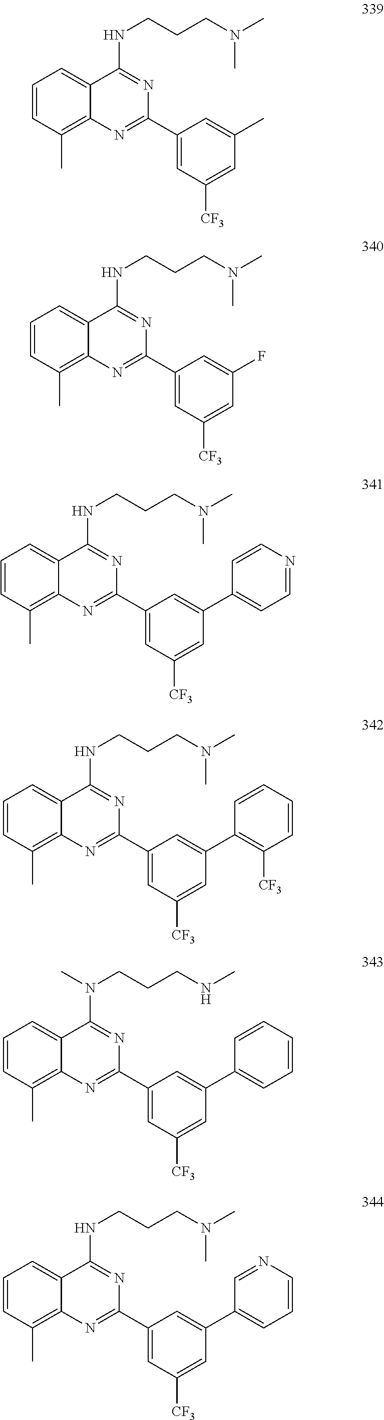

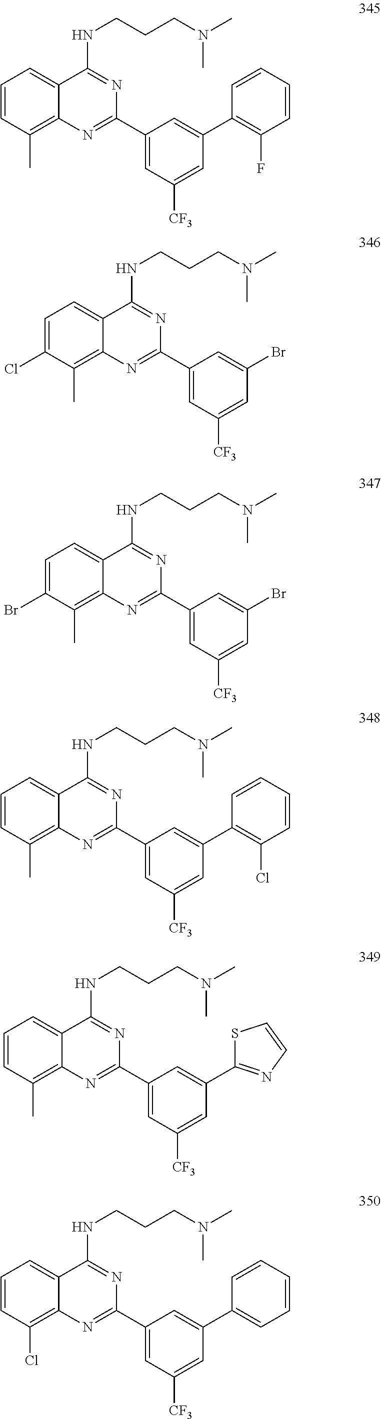

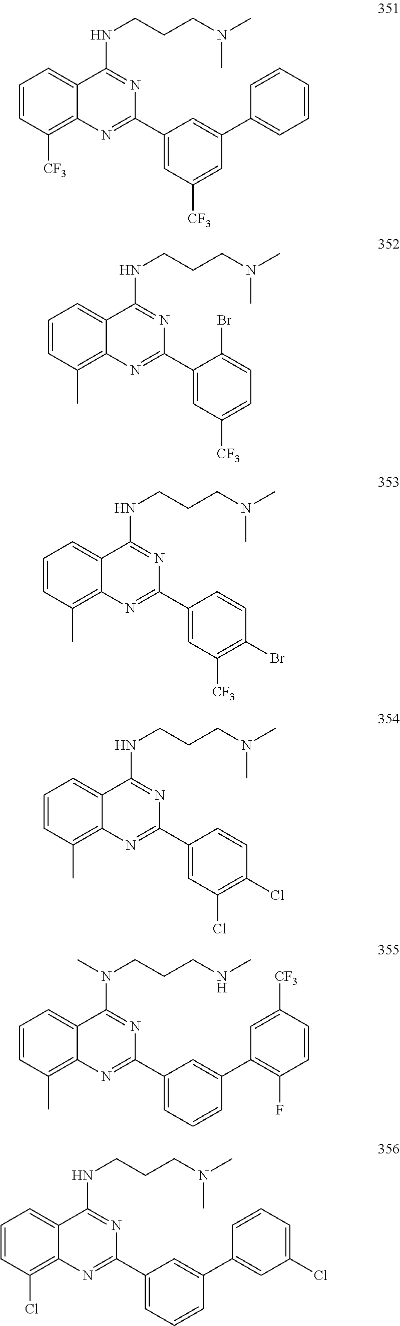

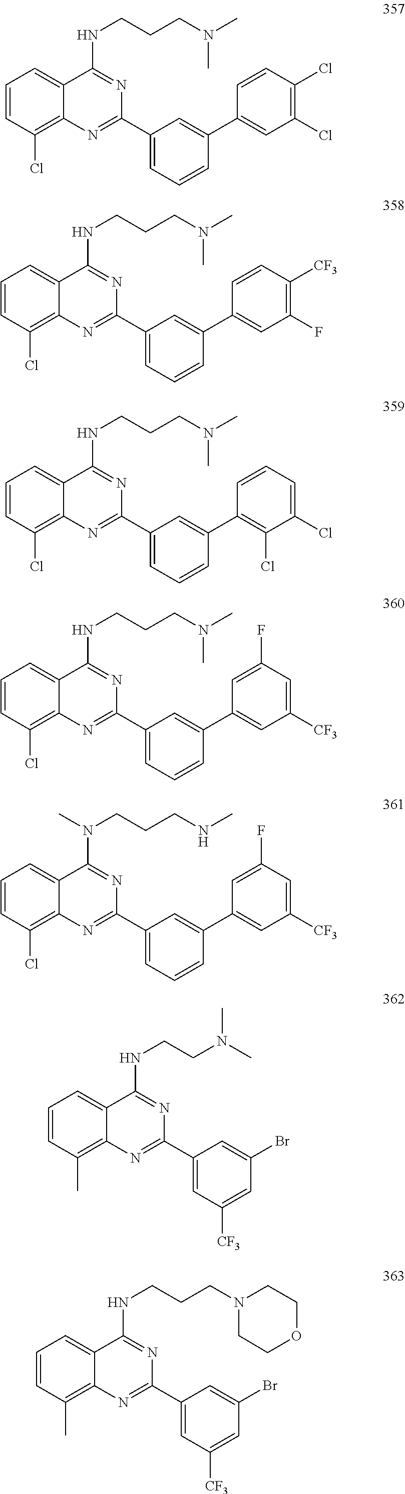

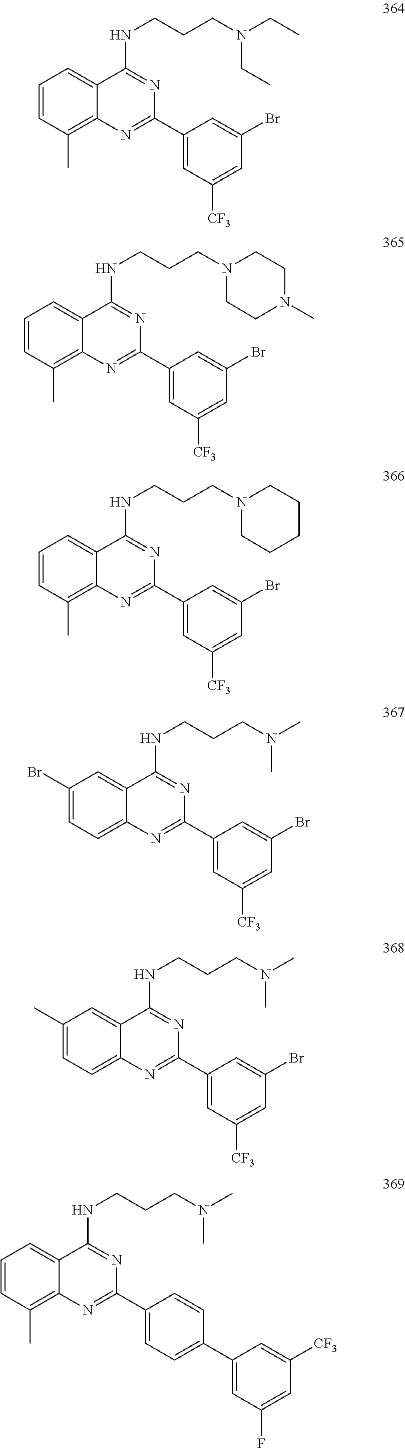

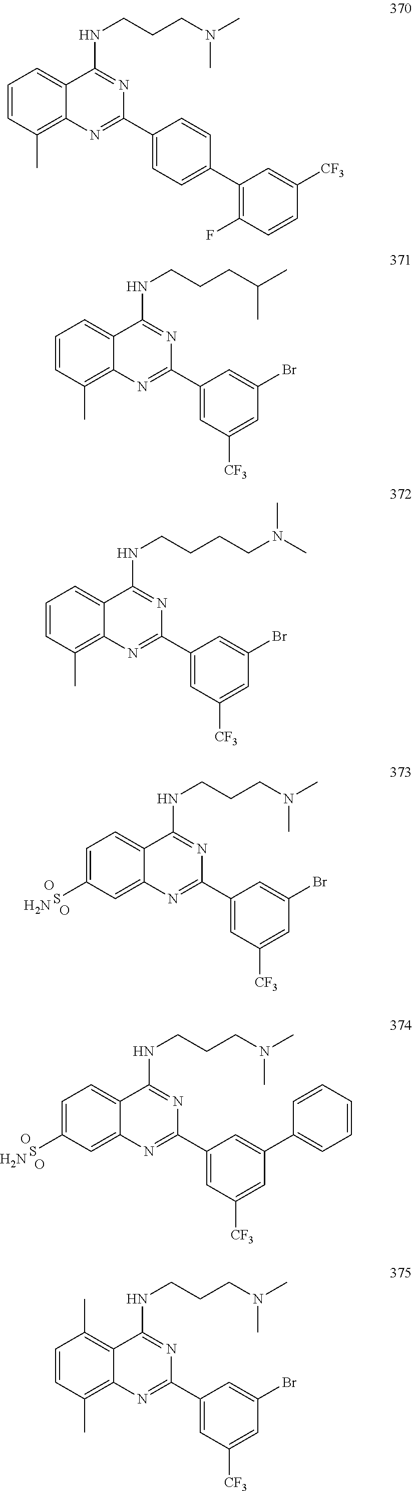

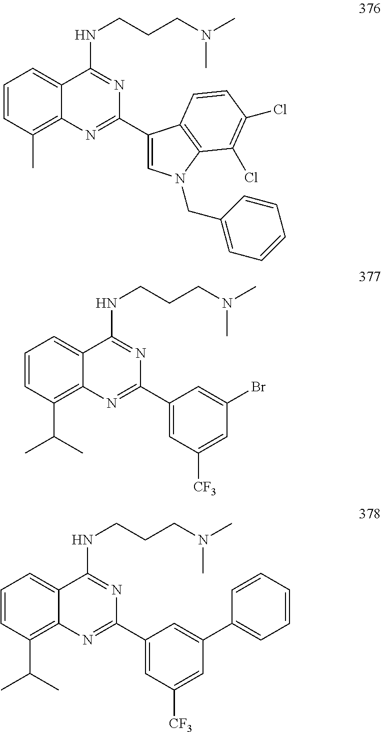

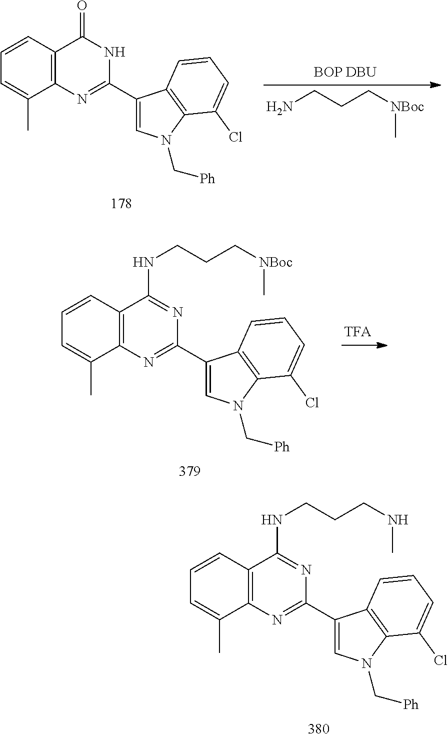 [SMILES: Cc1cccc2c(=O)[nH]c(-c3cn(Cc4ccccc4)c4c(Cl)cccc34)nc12 ; "BOP DBU" ; CN(CCCN)C(=O)OC(C)(C)C ; Cc1cccc2c(NCCCN(C)C(=O)OC(C)(C)C)nc(-c3cn(Cc4ccccc4)c4c(Cl)cccc34)nc12 ; CNCCCNc1nc(-c2cn(Cc3ccccc3)c3c(Cl)cccc23)nc2c(C)cccc12]

178 was converted to 379 in a manner similar to that described above for 255. Mass spectrum (ESI⁺): m/z=570 [M+1]observed.

A 10 mL pear-shaped flask was charged with 379 (0.020 g, 0.035 mmol, 1 eq) and a stir bar. The flask was evacuated and back-filled with Ar (×3). Anhydrous DCM (1.5 mL) was added and the reaction was cooled to 0° C. TFA (0.2 mL, 0.289 g, 2.6 mmol, large excess) was added dropwise. After 30 min the volatiles were removed via rotary evaporation. The residue was diluted with water and adjusted to pH≈9 with 1N NaOH. The mixture was extracted with EtOAc (×1), and the layers were separated. The organic layer was washed with water (×1), brine (×1), and dried over Na₂SO₄. The solids were filtered off, and the volatiles were removed via rotary evaporation. Purification via plug filtration through silica gel eluting sequentially with 1:1 EtOAc/DCM and 20% MeOH in DCM yielded 0.0125 g (0.027 mmol, 76% yield) of 380. ¹H NMR (CDCl₃, 300 MHz) δ 8.96-8.93 (m, 1H), 8.21 (s, 1H), 7.65 (broad s, 1H), 7.58-7.53 (m, 2H), 7.29-7.20 (m, 7H), 7.10-7.08 (m, 2H), 5.89 (s, 2H), 3.88 (broad s, 2H), 2.89-2.85 (m, 2H), 2.81 (s, 3H), 2.43 (s, 3H), 2.00-1.97 (m, 2H). Mass spectrum (ESI⁺): m/z=470 [M+1].

The following quinazolines were synthesized from the appropriate quinazolinone in a manner similar to that described for 380 above. The amines were purchased from the vendor indicated and used without purification.

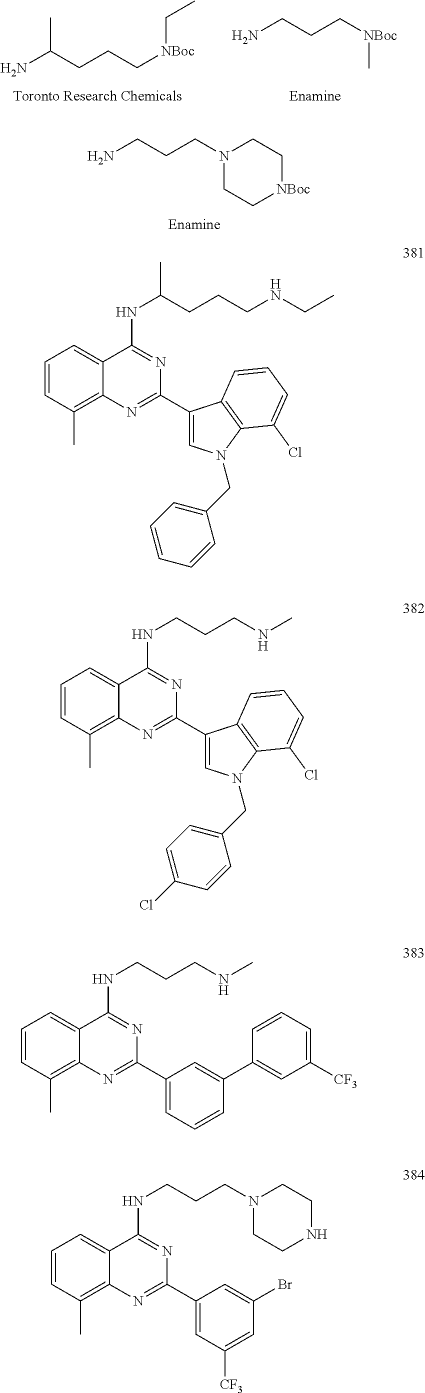

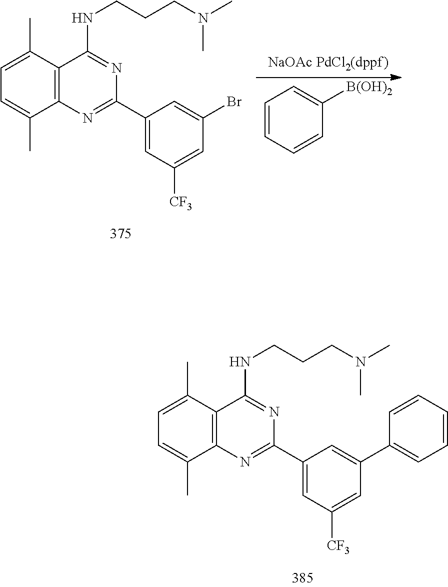

A 20 mL screw-top vial was charged with 375 (0.020 g, 0.042 mmol, 1 eq),

phenylboronic acid (0.086 g, 0.071 mmol, 1.7 eq), NaOAc (0.0174 g, 0.21 mmol, 5.1 eq), and a stir bar. 1,4-dioxane/water (10:1, 5 mL) was added, and the vial was sealed with a septum. The resulting stirred mixture was sparged with Ar (10 min). PdCl₂(dppf) (0.0061 g, 0.008 mmol, 0.2 eq) was added. The vial was sealed with the lid, and the reaction was heated to 90° C. overnight. After cooling to room temperature the mixture was diluted with MTBE/water. After stirring for 10 min the mixture was filtered. The layers were separated. The organic layer was washed with water (×3), brine (×1), and dried over Na₂SO₄. The solids were filtered off and the volatiles were removed via rotary evaporation. Purification via flash chromatography on silica gel eluting with 0-20% solvent B (B=5% conc. NH₃(aq) in MeOH) in EtOAc yielded impure product. Purification via prep TLC eluting with 14% (×1) and 17% (×1) MeOH in DCM yielded 0.0039 g (0.008 mmol, 20% yield) of 385. ¹H NMR (CDCl₃, 300 MHz) δ 9.02 (s, 1H), 8.83 (s, 1H), 7.89 (s, 1H), 7.74-7.72 (m, 2H), 7.53-7.42 (m, 5H), 7.08-7.06 (m, 1H), 3.89-3.88 (m, 2H), 2.84 (d, J=3.0 Hz, 3H), 2.73 (d, J=3.3 Hz, 3H), 2.56-2.55 (m, 2H), 2.28 (d, J=3.3 Hz, 6H), 1.96-1.94 (m, 2H). Mass spectrum (ESI⁺): m/z=479 [M+1].

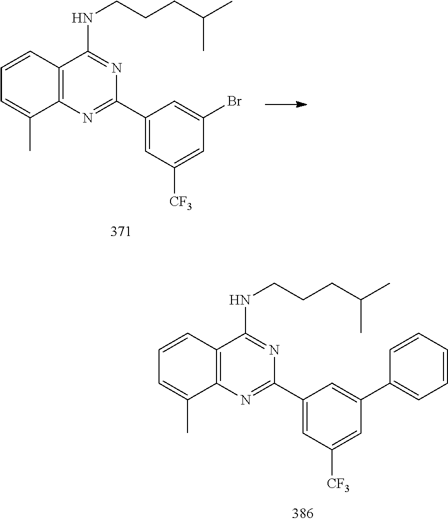 [SMILES: Cc1cccc2c(NCCCC(C)C)nc(-c3cc(Br)cc(C(F)(F)F)c3)nc12 ; Cc1cccc2c(NCCCC(C)C)nc(-c3cc(-c4ccccc4)cc(C(F)(F)F)c3)nc12]

386 was synthesized from 371 in a manner similar to that described for 385 above.

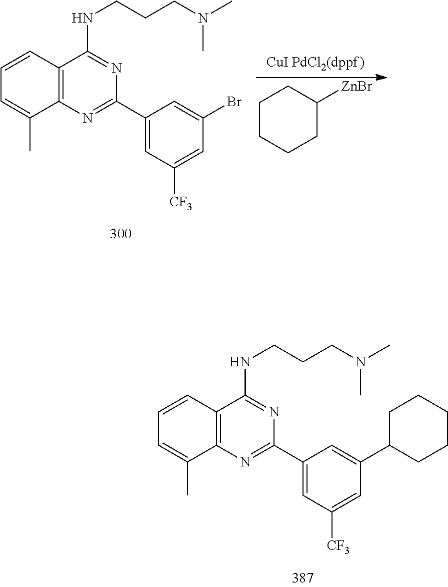

An oven-dried 2-necked, 50 mL round bottom flask and stir bar were cooled to room temperature under vacuum. 300 (0.020 g, 0.042 mmol, 1 eq) was added, and the flask was evacuated and back-filled with Ar (×3). Anhydrous THF (4 mL) was added. CuI (0.0037 g, 0.019 mmol, 0.45 eq) and PdCl₂(dppf) (0.0047 g, 0.006 mmol, 0.15 eq) were added with stirring. Cyclohexylzinc bromide (Alfa Aesar, 0.5M in THF, 0.13 mL, 0.064 mmol, 1.5 eq) was added via slow dropwise addition. The reaction was stirred overnight. LCMS indicated only partial conversion. The reaction was heated to 40° C. No progress was evident after 4 h. Additional CuI (0.0074 g), PdCl₂(dppf) (0.0094 g), and Cyclohexylzinc bromide (0.26 mL) were added. The reaction was stirred overnight at 40° C. A small amount of progress was evident. The temperature was increased to 50° C. After 4 days at 50° C. no progress was evident. The reaction was cooled to room temperature and diluted with MTBE/water. After stirring for 10 min the mixture was filtered, rinsing with MTBE. The layers were separated. The organic layer was washed with water (×3), brine (×1), and dried over Na₂SO₄. The solids were filtered off, and the volatiles were removed via rotary evaporation. Purification via prep TLC eluting with EtOAc/MeOH/conc. NH₃(aq) (90:9.5:0.5) (×3) yielded 0.0051 g (0.011 mmol, 25% yield) of 387. ¹H NMR (CDCl₃, 300 MHz) δ 8.67-8.62 (m, 2H), 8.44 (broad s, 1H), 7.89 (s, 1H), 7.56-7.48 (m, 3H), 7.32-7.28 (m, 1H), 3.89 (broad s, 2H), 2.79 (s, 3H), 2.70-2.62 (m, 3H), 2.38 (s, 6H), 1.99-1.88 (m, 6H), 1.58-1.43 (m, 4H), 1.32-1.25 (m, 2H). Mass spectrum (ESI⁺): m/z=471 [M+1].

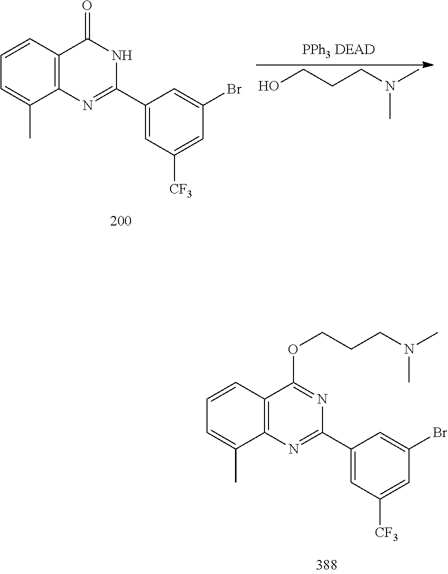

A 2-necked, 50 mL round bottom flask was charged with PPh₃ (0.1232 g, 0.47 mmol, 2 eq) and a stir bar. The flask was evacuated and back-filled with Ar (×3). Anhydrous 1,4-dioxane (10 mL) was added. DEAD (56 μL, 0.0614 g, 0.35 mmol, 1.5 eq) was added dropwise to the resulting stirred solution. After 5 min 3-dimethylamino-1-propanol (Alfa Aesar, 55 μL, 0.0485 g, 0.47 mmol, 2 eq) was added dropwise. 200 (0.090 g, 0.23 mmol, 1 eq) was added, and the reaction was stirred overnight. The volatiles were removed via rotary evaporation. The residue was partitioned between EtOAc/water, and the layers were separated. The organic layer was washed with water (×2), brine (×1), and dried over Na₂SO₄. The solids were filtered off, and the volatiles were removed via rotary evaporation. Purification via flash chromatography on silica gel eluting with 0-20% solvent B (B=5% conc. NH₃(aq) in MeOH) in EtOAc yielded the impure product. Purification via flash chromatography on silica gel eluting with 0-10% MeOH in DCM yielded 0.0030 g, (0.0064 mmol, 2.7% yield) of 388. ¹H NMR (CDCl₃, 300 MHz) δ 8.93 (s, 2H), 8.81 (s, 1H), 8.07-8.02 (m, 1H), 7.85 (s, 1H), 7.72-7.67 (m, 1H), 7.49-7.45 (m, 1H), 4.77-4.71 (m, 2H), 2.81 (s, 3H), 2.61-2.53 (m, 2H), 2.38 (s, 6H), 2.19-2.12 (m, 2H). Mass spectrum (ESI⁺): m/z=468/470 [M+1].

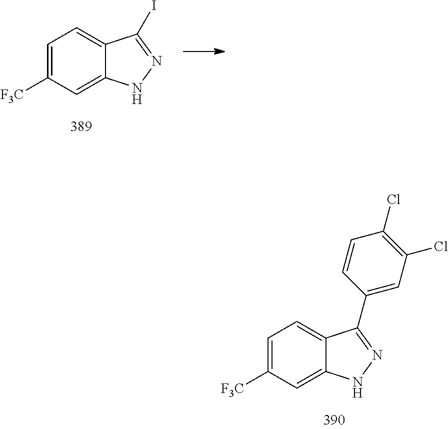 [SMILES: FC(F)(F)c1ccc2c(I)n[nH]c2c1 ; FC(F)(F)c1ccc2c(-c3ccc(Cl)c(Cl)c3)n[nH]c2c1]

To the iodo compound 389 (30 mg, 0.16 mmol), in dioxane\water (3\1 ml), 3,4-dichloro phenyl boronic acid (37 mg, 0.19 mmol), sodium acetate (62 mg, 0.76 mmol) and PdCl₂(dppf) (23 mg, 0.03 mmol) were added. Then the reaction mixture was heated to 100° C. for 16 h. Then reaction mixture was cooled and diluted with ethyl acetate. Organic layer was separated and washed with water, brine and dried. Crude residue was column chromatographed to yield 390 (3-(3,4-dichlorophenyl)-6-(trifluoromethyl)-1H-indazole) in 65% yield. ¹H NMR (CDCl₃): 10.41 (br s, 1H), 8.1 (m, 2H), 7.68-7.72 (m, 2H), 7.60 (m, 1H), 7.53 (d, 1H). Mass spectrum (ESI⁺): m/z=331 [M+1].

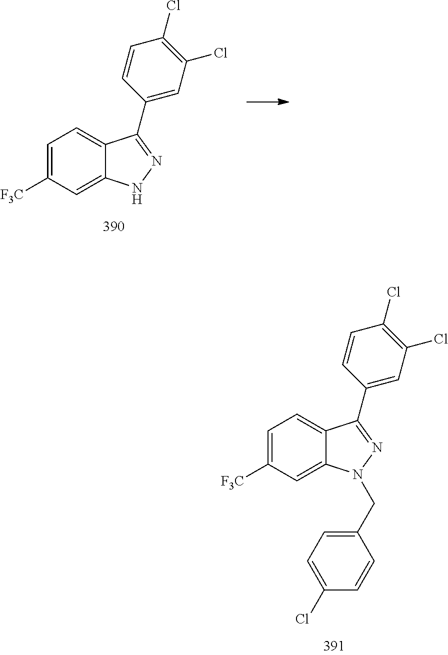 [SMILES: FC(F)(F)c1ccc2c(-c3ccc(Cl)c(Cl)c3)n[nH]c2c1 ; FC(F)(F)c1ccc2c(-c3ccc(Cl)c(Cl)c3)nn(Cc3ccc(Cl)cc3)c2c1]

To the dichloro compound 390 (16 mg, 0.05 mmol) in DMF (3 ml), cesium carbonate (19.5 mg, 0.06 mmol) was added. After stirring for 10 minutes at room temperature, 4-chlorobenzyl bromide (12 mg, 0.06 mmol) was added and stirred overnight at room temperature. Then reaction mixture was quenched with water, extracted with ethyl acetate. Organic layer was washed with water, brine and dried. Crude residue was column chromatographed to yield 391 (1-(4-chlorobenzyl)-3-(3,4-dichlorophenyl)-6-(trifluoromethyl)-1H-indazole) in 65% yield. ¹H NMR (CDCl₃): 8.1 (d, 2H), 7.82 (d, 1H), 7.68 (s, 1H), 7.58 (d, 1H), 7.44 (d, 1H), 7.25-7.32 (m, 3H), 7.18 (d, 1H), 5.61 (s, 2H). Mass spectrum (ESI⁺): m/z=455 [M+1].

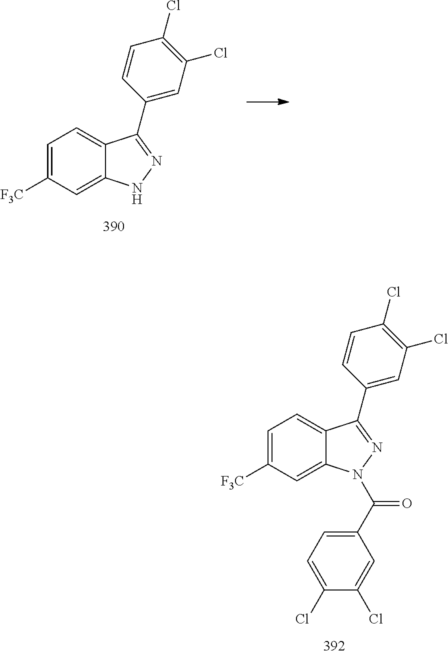 [SMILES: FC(F)(F)c1ccc2c(-c3ccc(Cl)c(Cl)c3)n[nH]c2c1 ; O=C(c1ccc(Cl)c(Cl)c1)n1nc(-c2ccc(Cl)c(Cl)c2)c2ccc(C(F)(F)F)cc21]

To the dichloro compound 390 (26 mg, 0.08 mmol) in DMF (3 ml), DMAP (10 mg, 0.08 mmol) was added followed by trimethylamine (16 mg, 0.16 mmol). After stirring for 10 minutes at room temperature, 3, 4-dichlorobenzoyl chloride (17 mg, 0.08 mmol) was added and stirred overnight at room temperature. Then reaction mixture was quenched with water, extracted with ethyl acetate. Organic layer was washed with water, brine and dried. Crude residue was column chromatographed to yield 392 ((3,4-dichlorophenyl)(3-(3,4-dichlorophenyl)-6-(trifluoromethyl)-1H-indazol-1-yl)methanone) in 72% yield. ¹H NMR (CDCl₃): 8.88 (s, 1H), 8.31 (s, 1H), 8.16-8.0 (m, 3H), 7.82-7.76 (m, 2H), 7.60 (d, 2H). Mass spectrum (ESI⁺): m/z=503 [M+1].

### 5-Chloro-3-(3,4-dichlorophenyl)-1H-indole

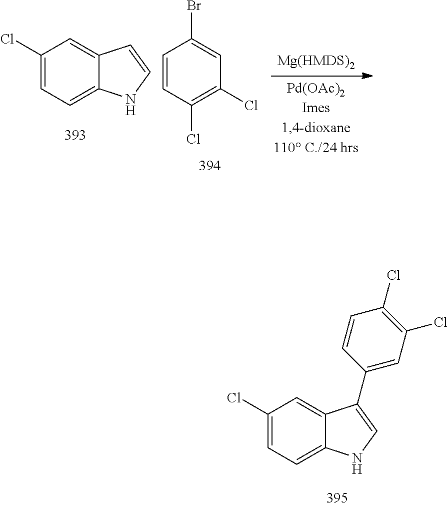

In a 40 ml septum screw-top vial with stir bar was placed 393, (5-chloroindole, 1.0 g, 6.6 mmol, 1 eq) and flushed with argon. 1,4-Dioxane (10 ml) was added and then magnesium bis(hexamethyldisilazide)(Aldrich, 5 g, 14.4 mmol, 2.2 eq). This mixture was heated to 65° C. for 30 min.

In a separate screw-top vial was placed argon, Pd(OAc)₂ (Aldrich, 37.04 mg, 0.165 mmol, 2.5%), and Imes [(1,3-bis-(2′,4′,6′-trimethylphenyl)imidazolium chloride), Aldrich, 56.26 mg, 0.165 mmol, 2.5%]followed by 1,4-dioxane (10 ml) and vigorous stirring with gentle heating. To this Pd(OAc)₂ mixture was added 394, [3,4-dichlorobromobenzene (Combi-Blocks, 1.79 g, 1.02 ml, 7.92 mmol, 1.2 eq)]. This mixture was then added to the vial containing the indole magnesium salt. Finally, the entire mixture was heated to 110° C. for 24 hours. Upon completion, the reaction was diluted with dichloromethane (200 ml) and vacuum filtered through Celite. The solvent was removed by rotary evaporation to give a reddish-black residue. This residue as purified by first passing through a pad of silica gel eluting with 9/1 hexane/DCM. Subsequent purification steps included flash chromatography on silica gel eluting with a solvent gradient of 3-10% EtOAc in hexane. After removing the solvent by rotary evaporation the resulting solid was triturated with Et₂O and dried under vacuum to provide 0.51 grams of 395 (26% yield) as a white solid. ¹H NMR (DMSO-d₆, 400 MHz) δ 11.67 (bs, 1H, NH), 7.94 7.94 (d, 2.8 hz, 1H), 7.88 (d, 1.96 hz, 1H), 7.83 (d, 1.95 hz, 1H), 7.69-7.71 (m, 1H), 7.65 (d, 8.2 hz, 1H), 7.48 (d, 8.98 hz, 1H), 7.18 (dd, 8.59 hz, 1.95 hz, 1H).

### 5-(3-(trifluoromethyl)phenyl)-1H-indole

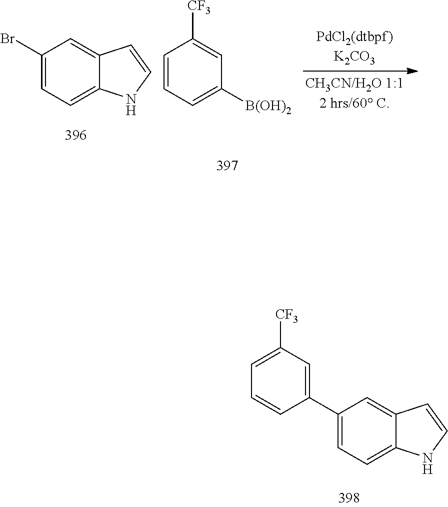

In a 40 ml septum screw-top vial with stir bar was added 396 (5-bromoindole, 1 g, 5.101 mmol, 1 eq), 397 (3-trifluoromethyl phenylboronic acid, 1.163 g, 6.12 mmol, 1.2 eq), PdCl₂(dtbpf) [1,1′ Bis(di-tert-butylphosphino)ferrocene]dichloropalladium II], Aldrich, 33.24 mg, 0.051 mmol, 1 mol %), K₂CO₃ (1.057 g, 7.65 mmol, 1.5 eq) and 1:1 CH₃CN/water (11 ml). The mixture was heated to 60° C. for 2 hrs and then cooled to room temp. The lower layer that formed while cooling was removed with a pipette. Purification with flash chromatography on silica gel eluting with 95/5 hexane/EtOAc followed by additional silica gel chromatography eluting with a gradient 5-10% DCM in hexane provided 398 (720 mg, 54% yield). ¹H NMR (DMSO-d₆, 400 MHz) δ 11.2 (bs, 1H), 7.93-7.99 (m, 3H), 7.65-7.69 (m, 2H), 7.52-7.54 (m, 1H), 7.42-7.48 (m, 2H), 6.54 (s, 1H).

### (E)-3-(3,4-dichlorostyryl)-6-fluoro-1-(2-(4-methylpiperazin-1-yl)ethyl-1H-indole

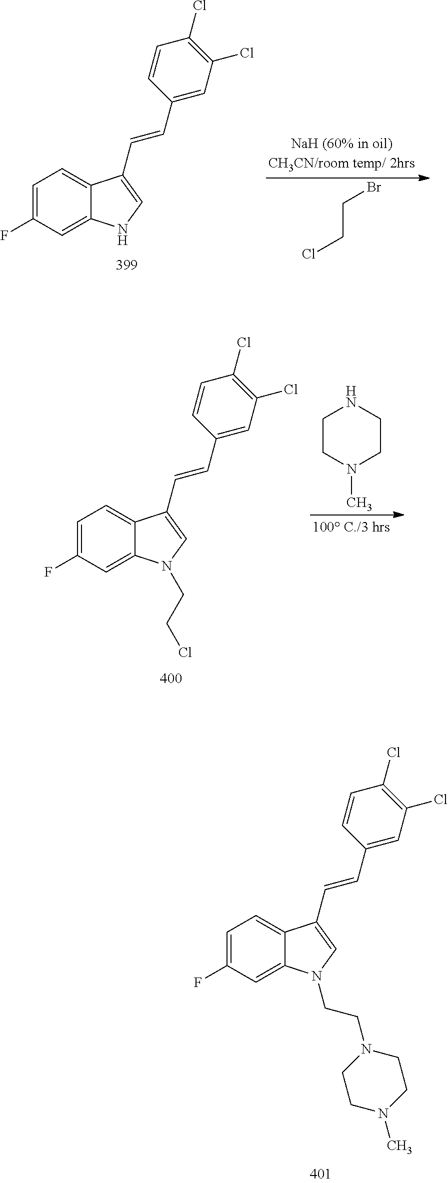

(E)-1-(2-chloroethyl)-3-(3,4-dichlorostyryl)-6-fluoro-1H-indole. ¹H NMR (400 MHz, DMSO-d₆): δ 11.40 (bs, 1H, NH), 8.74-8.78 (m, 1H), 8.34-8.40 (m, 1H), 7.96-8.06 (m, 2H), 7.68 (s, 1H), 7.50-7.58 (d, 1H), 7.32-7.38 (m, 1H), 7.18-7.24 (m, 1H), 7.06-7.14 (d, 1H), 6.98-7.20 (m, 1H).

Compound 399 was prepared in a similar manner as compound 130.

In a dry 250 ml round bottom flask with stir bar was placed 399 (200 mg, 0.653, 1 eq) and dissolved with 10 ml of anhydrous CH₃CN. The flask was flushed with argon and NaH (60% in oil) was added (450 mg of 60% in oil), 11.15 mmol, 17 eq—large excess intended). The mixture was stirred at room temp for 45 min and then bromochloroethane (d=1.723 g/ml, 0.5 ml, 6 mmol, 9.19 eq) was added dropwise. After 2 hours, the reaction was quenched by very careful addition of (drop-by-drop) water and eventually partitioned with water/EtOAc followed by a brine wash. Dried with Na₂SO₄, filtered and removed the solvent with rotary evaporation to give an amber color residue. Purification with Si-gel flash chromatography eluting with 3% EtOAc in hexane provided the desired chloroethylindole intermediate compound 400. (200 mg, 83% yield). ¹H NMR (DMSO-d₆, 400 MHz) δ 8.05-8.09 (m, 1H), 7.89 (s, 1H), 7.74 (s, 1H), 7.49-7.59 (m, 4H), 7.00-7.11 (m, 2H), 4.6 (t, 2H), 4.0 (t, 2H).

### (E)-3-(3,4-dichlorostyryl)-6-fluoro-1-(2-(4-methylpiperazin-1-yl)ethyl-1H-indole

Placed the intermediate compound 400 (100 mg, 0.271 mmol, 1 eq) in a 40 ml septum screw-top vial and flushed with argon. Added the 4-methylpiperazine (0.903 g, 1.0 ml, 9.0 mmol, 33 eq) and placed on a hotplate-stirrer at 100° C. for 3 hrs and then stir overnight at room temperature. The reaction was taken up in EtOAc and rotary evaporated to give the crude residue. The residue was partitioned with EtOAc and 0.5M HCl and further extracted the aqueous with EtOAc. The aqueous layer was made basic with sat'd. aq. Na₂CO₃ and the product extracted into EtOAc. The organic layer was then dried (Na₂SO₄), filtered and rotary evaporated to give the crude product. Purification with flash chromatography on silica gel eluting with 5% MeOH in dichloromethane provided 401. ¹H NMR (DMSO-d₆, 400 MHz) δ 8.02-8.06 (m, 1H), 7.88 (s, 1H), 7.69 (s, 1H), 7.41-7.58 (m, 4H), 6.97-7.08 (m, 2H), 4.27 (t, 2H), 2.63 (t, 2H), 2.44 (bs, 4H), 2.28 (bs, 4H), 2.12 (s, 3H).

### 5-Bromo-N-(3,4-dichlorophenyl)-1H-indole-3-carboxamide

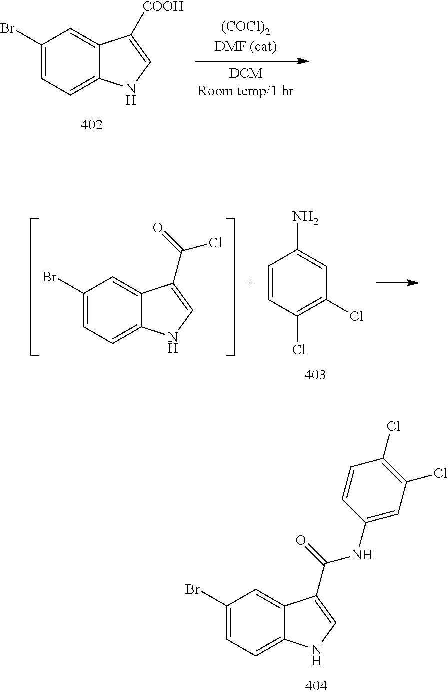

In a flame dried 250 ml round bottom flask with stir bar was placed argon, 402 (1 (5-bromoindole-3-carboxylic acid, 0.5 g, 2.08 mmol, 1 eq) and 20 ml of anhydrous dichloromethane (DCM). Oxalyl chloride (2M in DCM, 2.08 ml, 4.16 mmol, 2 eq) was added. Then 1-2 drops of DMF were added as a catalyst and stirred for 1 hr at room temp. The reaction was placed on a dry rotary evaporator to remove all volatiles to give a solid residue. To the residue was added 20 ml anhydrous DCM and taken up in syringe needle and added dropwise to a separate round bottom flask containing 403, (3,4-dichloroaniline, 405 mg, 2.5 mmol, 1.2 eq), Et₃N (0.725 ml) and 50 ml of DCM. Stir overnight at room temperature. The solvent was then removed with a rotary evaporator and the residue was partitioned with EtOAc and water. The EtOAc layer was washed with 1N HCl (3×50 ml), sat'd. NaHCO₃, brine and dried (Na₂SO₄), filtered and rotary evaporated to give a solid residue. Triturate with EtOAc and dried with vacuum to give 404 (295 mg, 37% from the indole). ¹H NMR (DMSO-d₆) δ 12.0 (s, 1H), 10.03 (s, 1H), 8.33-8.36 (m, 2H), 8.17-8.18 (d, J=2.34 hz, 1H), 7.71-7.73 (m, 1H), 7.59 (d, J=8.98 hz, 1H), 7.48 (d, J=8.59 hz, 1H), 7.32-7.35 (m, 1H).

### 5-Bromo-3-(3,4-dichlorophenethyl)-1H-indole

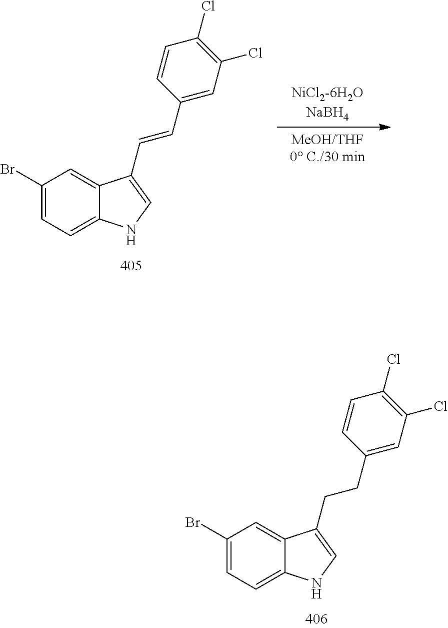 [SMILES: Clc1ccc(/C=C/c2c[nH]c3ccc(Br)cc23)cc1Cl ; Clc1ccc(CCc2c[nH]c3ccc(Br)cc23)cc1Cl]

Compound 405 was prepared in a similar manner as compound 130. ¹H NMR (400 MHz, DMSO-d₆): δ 8.04-8.12 (m, 1H), 7.89 (s, 1H), 7.81 (s, 1H), 7.42-7.60 (m, 4H), 7.30-7.38 (m, 2H), 7.14-7.20 (m, 2H), 7.06-7.13 (d, J=16.8 hz, 1H), 6.98-7.04 (m, 1H), 5.41 (s, 2H).

In a 250 ml round bottom flask with stir bar was placed the styryl indole 405 (634 mg, 1.737 mmol, 1 eq) and 40 ml of 2:1 MeOH/THF. Added the green colored NiCl₂.6H₂O (330.34 mg, 1.3898 mmol, 0.8 eq) and cooled in an ice bath. Then added the NaBH₄ very slowly. After 30 min, the reaction was quenched with water and extracted with EtOAc. The organic layer was dried (Na₂SO₄), filtered and suspended onto Si-gel. Silica gel chromatography eluting with a gradient of 0-1% hexane/EtOAc provided 406. ¹H NMR (DMSO-d₆, 400 MHz) δ 11.0 (bs, 1H), 7.69 (d, J=1.83 hz, 1H), 7.55 (d, J=2 hz, 1H), 7.50 (d, J=8.2 hz, 1H), 7.29 (d, J=8.6 hz, 1H), 7.24 (m, 1H), 7.15 (m, 2H), 2.90-3.01 (m, 4H).

### N-((5-bromo-1H-indol-3-yl)methyl-3,4-dichloroaniline

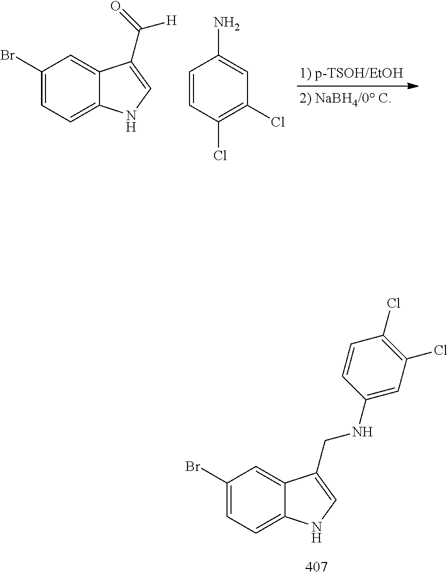

In a 250 ml round bottom flask placed p-toluenesulfonic acid (153.6 mg, 0.892 mmol, 0.1 eq), 3,4-dichloroaniline (1.445 g, 8.92 mmol, 1 eq), 5-bromoindole-3-carboxaldehyde (2.0 g, 8.92 mmol, 1 eq) and 80 ml absolute EtOH. The resulting mixture was stirred overnight at room temperature. Cooled (ice bath) and then added NaBH₄ (1.021 g, 27 mmol, 3 eq) and stirred at 0° C. for 1 hr. Then added 100 ml water and extracted with EtOAc, washed with brine, dried (Na₂SO₄), filtered and solvent removed by rotary evaporation. Silica gel chromatography (95/5 hexanes/EtOAc) provided 407 as a thick oil which solidified under vacuum. ¹H NMR (DMSO-d₆, 400 MHz) δ 11.1 (s, 1H), 7.82 (d, 1H), 7.40 (d, 1H), 7.32 (d, J=8.54 hz, 1H), 7.16-7.24 (m, 2H), 6.84 (d, 1H), 6.62-6.68 (m, 1H), 6.46-6.5 (m, 1H).

### 2-amino-1-(5-chloro-3-(3,4-dichlorophenyl)-1H-indol-1-yl)ethan-1-one hydrochloride

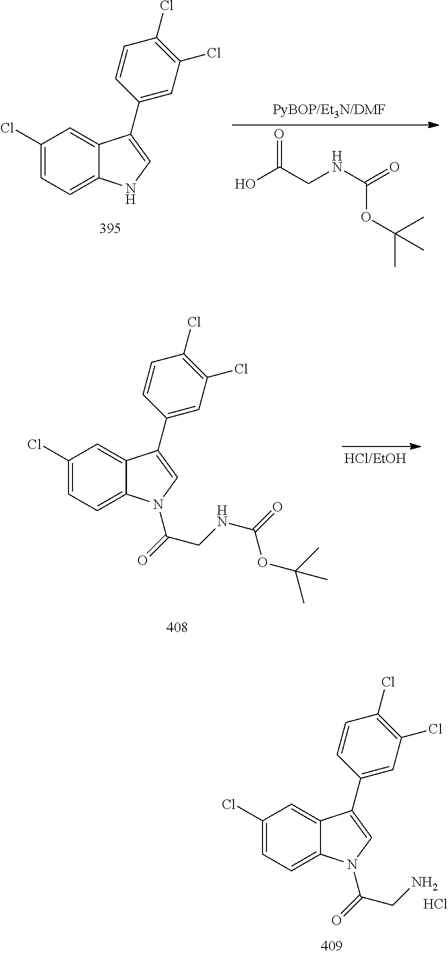 [SMILES: Clc1ccc2[nH]cc(-c3ccc(Cl)c(Cl)c3)c2c1 ; CC(C)(C)OC(=O)NCC(=O)O ; CC(C)(C)OC(=O)NCC(=O)n1cc(-c2ccc(Cl)c(Cl)c2)c2cc(Cl)ccc21 ; NCC(=O)n1cc(-c2ccc(Cl)c(Cl)c2)c2cc(Cl)ccc21 ; Cl]

### tert-butyl (2-(5-chloro-3-(3,4-dichlorophenyl)-1H-indol-1-yl)-2-oxoethyl)carbamate

In a 250 round bottom flask added 395 (5-chloro-3-(3,4-dichlorophenyl)-1H-indole, 500 mg, 1.686 mmol, 1 eq), N-Boc glycine (1 mg, 2.107 mmol, 1.25 eq), PyBOP (Aldrich, 1.316 grams, 2.529 mmol, 1.5 eq), Triethylamine (341.2 mg, 0.47 ml, 3.372 mmol, 2 eq) and 2 ml of DMF. Stirred for 3 hrs at rt. Poured into 300 ml water and extracted with EtOAc (3×200 ml). TLC (80/20 hexanes/EtOAc) of the EtOAc layer showed a major product spot Rf=0.45 Dried (Na₂SO₄), filtered, concentrated to give a clearish oil (1.78 grams) as crude product. Purification using a Si-gel pad and eluting with 80/20 hexanes/EtOAc produced the desired Boc protected product 408 as a white solid intermediate. ¹H NMR (400 MHz) δ 8.48-8.40 (m, 2H), 8.06-8.02 (m, 1H), 7.90-7.87 (m, 1H), 7.82-7.54 (m, 2H), 7.51-7.44 (M, 1H), 7.42-7.36 (m, 1H), 4.52-4.50 (m, 2H), 1.49-1.40 (s, 9H).

### 2-amino-1-(5-chloro-3-(3,4-dichlorophenyl)-1H-indol-1-yl)ethan-1-one hydrochloride

In a 40 ml screw-top vial with stir bar, placed the Boc-glycine indole intermediate 408 (800 mg, 1.763 mmol, 1 eq), 20 ml EtOH and then conc. HCl (0.735 ml, 8.82 mmol, 5 eq). Heated the mixture to 70° C. for 2 h and stirred overnight. Evaporated the solvent in vacuo to give a white solid. Rinsed the solid with water and then triturated with EtOAc to give 409. ¹H NMR (400 MHz); δ 8.54-8.68 (bs, 3H, NH3), 8.42-8.46 (m, 2H), 8.04-8.07 (d, 1H), 7.92-7.95 (d, 1H), 7.776-7.84 (m, 2H), 7.52-7.56 (m, 1H), 4.63 (s, 2H).

### 7-Chloro-2-methylquinazolin-4(3H)-one

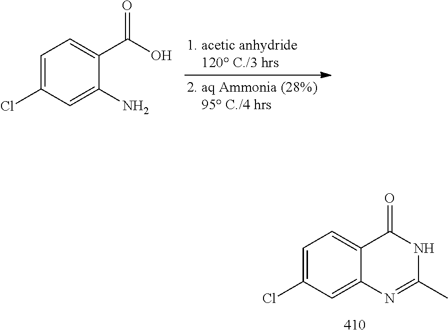

Placed 2-amino-4-chlorobenzoic acid (10.0 g, 58.28 mmol, 1 eq) in a 1000 ml round bottom flask with a stir bar. Added acetic anhydride (21.6 g, 20 ml, 211.5 mmol, 3.6 eq) and placed on a pre-heated oil bath at 120° C. for 3 h with stirring. This mixture was then placed on a rotary evaporator (50° C.) to remove excess acetic anhydride which gave a dry solid. To this was added 100 ml of ammonium hydroxide (28% NH₃) and heated to 95° C. for 4 h. Cooled, vacuum filtered and washed the solid with water (500 ml), then Et₂O (100 ml), and dried in vacuo to give 10 grams of 410. ¹H NMR (400 MHz, DMSO-d₆), S 12.3 (bs, 1H), 8.0-8.1 (m, 1H), 7.61 (s, 1H), 7.4-7.5 (m, 1H), 2.35 (s, 3H).

### (E)-7-Chloro-2-(3,4-dichlorostyryl)quinazolin-4(3H)-one

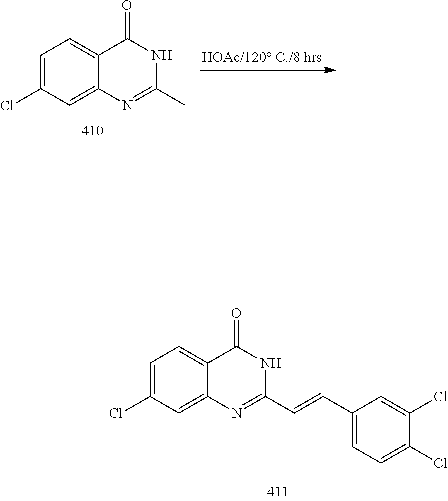 [SMILES: Cc1nc2cc(Cl)ccc2c(=O)[nH]1 ; O=c1[nH]c(/C=C/c2ccc(Cl)c(Cl)c2)nc2cc(Cl)ccc12]

7-Chloro-2-methylquinazolin-4(3H)-one (1.0 g, 5.14 mmol, 1 eq) was placed in a 40 ml screw-top vial with a stir bar. Added 3,4-dichlorobenzaldehyde (1.0 g, 5.71 mmol) was added and acetic acid (15 ml) was added to the mixture. The mixture was placed on a pre-heated hotplate-stirrer (120° C.) for 8 hrs. The reaction was cooled and the precipitate was vacuum filtered, washed with water, sat'd NaHCO₃ solution, water and a small amount of diethyl ether to remove the excess aldehyde. The solid was dried in a vacuum desiccator to give 1.80 grams of 411. (E)-7-chloro-2-(3,4-dichlorostyryl)quinazolin-4(3H)-one. ¹H NMR (400 MHz, DMSO-d₆): δ 12.4 (bs, 1H), 8.1 (d, 1H), 7.86-7.96 (m, 2H), 7.64-7.76 (m, 3H), 7.50-7.55 (m, 1H), 7.65 (d, 1H).

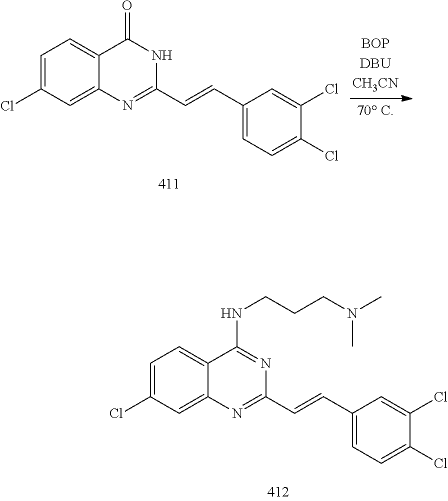 [SMILES: O=c1[nH]c(/C=C/c2ccc(Cl)c(Cl)c2)nc2cc(Cl)ccc12 ; CN(C)CCCNc1nc(/C=C/c2ccc(Cl)c(Cl)c2)nc2cc(Cl)ccc12]

### (E)-N1-(7-chloro-2-(3,4-dichlorostyryl)quinazolin-4-yl)-N3,N3-dimethylpropane-1,3-diamine

In a 250 ml round bottom flask was placed (E)-7-Chloro-2-(3,4-dichlorostyryl)quinazolin-4(3H)-one (3.0 g, 8.532 mmol, 1 eq) and BOP (Benzotriazol-1-yloxy)tris(dimethylamino)phosphonium hexafluorophosphate) (4.96 g, 11.22 mmol, 1.3 eq) with acetonitrile (120 ml). DBU (1,8-Diazabicyclo[5.4.0]undec-7-ene) (1.949 g, 1.915 ml, 12.8 mmol, 1.5 eq) was added to the mixture and stirred 10-15 minutes. The amine 3-(dimethylamino)-1-propylamine (1.4875 g, 1.876 ml, 12.8 mmol, 1.5 eq) was then added and the mixture was heated to 75° C. overnight. The solvent was removed in vacuo and the residue partitioned with EtOAc and brine. The organic layer was dried (Na2SO4), filtered and concentrated in vacuo. Purification by SiO₂ flash chromatography (9:1 DCM:MeOH) provided 412. ¹H NMR (400 MHz, DMSO-d₆): δ 8.44 (t, 1H, NH), 8.21 (d, J=8.5 hz, 1H), 8.0 (d, J=1.5 hz, 1H), 7.86 (d, J=16 hz, 1H), 7.62-7.74 (m, 3H), 7.48-7.54 (m, 1H), 7.21 (d, J=15.6 hz, 1H), 3.60-3.70 (m, 2H), 2.34-2.42 (t, 2H), 2.19 (s, 6H), 1.80-1.90 (m, 2H).

### 1-(5-chloro-3-(3,4-dichlorophenyl)-1H-indol-1-yl)-2-(dimethylamino)ethan-1-one

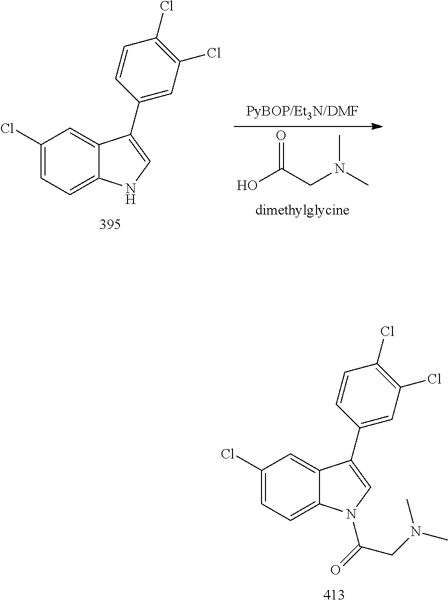

### 1-(5-chloro-3-(3,4-dichlorophenyl)-1H-indol-1-yl)-2-(dimethylamino)ethan-1-one

In a 40 ml screw-top vial with stirbar added 5-chloro-3-(3,4-dichlorophenyl)-1H-indole (500 mg, 1.686 mmol, 1 eq), Dimethylglycine (217 mg, 2.107 mmol, 1.25 eq), PyBOP (1.316 grams, 2.529 mmol, 1.5 eq), Triethylamine (341.2 mg, 0.47 ml, 3.372 mmol, 2 eq) and 2 ml of DMF. Stirred overnight (16 hrs) at room temp. Poured into 300 ml water and extracted with EtOAc (4×150 ml) with brine. Dried (Na₂SO₄), filtered, evaporated in vacuo to give the crude product which was suspended onto Si-gel. Flash chromatography (gradient; DCM-99/1 DCM/MeOH) provided 413. ¹H NMR (400 MHz, DMSO-d₆); δ 8.44 (d, 1H), 8.39 (s, 1H), 7.99-8.02 (m, 1H), 7.85-7.88 (m, 1H), 7.75-7.79 (m, 2H), 7.44-7.49 (m, 1H), 3.95 (s, 2H), 2.36 (s, 6H).

### 3-(5-chloro-3-(3,4-dichlorophenyl)-1H-indol-1-yl)-N,N-dimethylpropan-1-amine

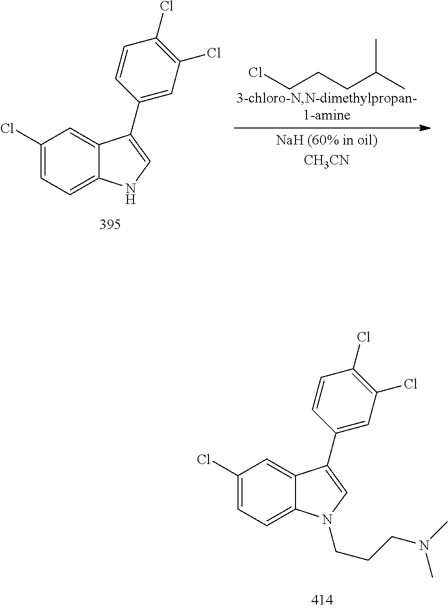

In a 250 ml round bottom flask with stir bar, dissolve 3-(5-chloro-3-(3,4-dichlorophenyl)-1H-indol-1-yl)-N,N-dimethylpropan-1-amine (200 mg, 0.6743 mmol, 1 eq) in 20 ml anhydrous acetonitrile and flushed with argon. Added NaH (60% in oil, 67.4 mg, 1.69 mmol, 2.5 eq). Stirred 45 minutes at room temp followed by heating to 50° C. in oil bath for 3 hrs. The solvent was removed in vacuo. The resulting crude material was purified via flash chromatography (eluting with 95/5 EtOAc/MeOH+0.05% NH₄OH) to produce 414. ¹H NMR (400 MHz, DMSO-d₆): δ 7.98-8.02 (s, 1H), 7.82-7.89 (m, 2H), 7.58-7.71 (m, 3H), 7.20-7.27 (m, 1H), 4.20-4.28 (t, 2H), 2.08-2.20 (m, 8H), 1.88-1.98 (m, 2H).

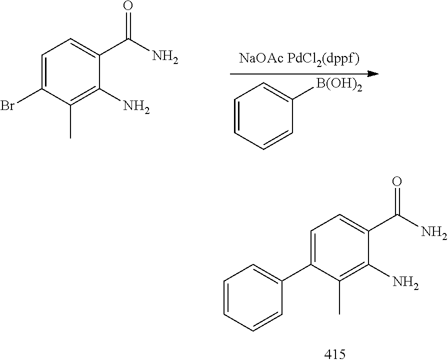 [SMILES: Cc1c(Br)ccc(C(N)=O)c1N ; OB(O)c1ccccc1 ; Cc1c(-c2ccccc2)ccc(C(N)=O)c1N]

A 20 mL screw-top vial was charged with 2-amino-4-bromo-3-methylbenzamide (0.065 g, 0.28 mmol, 1 eq), phenylboronic acid (0.0519 g, 0.43 mmol, 1.5 eq), NaOAc (0.105 g, 1.28 mmol, 4.5 eq), and a stir bar. 1,4-Dioxane/water (10:1, 5 mL) was added. The vial was sealed with a septum. The resulting stirred mixture was sparged with Ar (10 min). PdCl₂(dppf) (0.0415 g, 0.056 mmol, 0.2 eq) was added. The vial was closed with the lid. The reaction was heated to 90° C. overnight. After cooling to room temperature the reaction was diluted with water and adjusted to pH≈2-3 with 1N HCl. The mixture was filtered. The filtrate was extracted with DCM (×4). The combined DCM fractions were washed with brine (×1) and dried over Na₂SO₄. The solids were filtered off, and the volatiles were removed via rotary evaporation. Purification via flash chromatography on silica gel eluting with 0-60% EtOAc in hexanes yielded 0.0542 g, (0.24 mmol, 84% yield) of 415 as a pale yellow solid. Mass spectrum (ESI⁺): m/z=381 [M+1]observed.

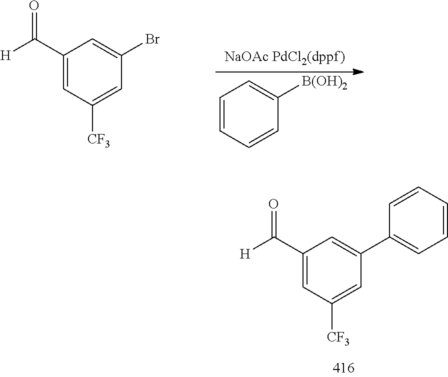 [SMILES: O=Cc1cc(Br)cc(C(F)(F)F)c1 ; OB(O)c1ccccc1 ; O=Cc1cc(-c2ccccc2)cc(C(F)(F)F)c1]

A 20 mL screw top vial was charged with 3-bromo-5-(trifluoromethyl)benzaldehyde (0.360 g, 1.4 mmol, 1 eq) and a stir bar. 1,4-Dioxane/water (10:1, 6 mL) was added. NaOAc (0.5253 g, 6.4 mmol, 4.5 eq) and phenylboronic acid (0.260 g, 2.1 mmol, 1.5 eq) were added. The vial was sealed with a septum, and the resulting stirred mixture was sparged with Ar (5 min). PdCl₂(dppf) (0.104 g, 0.14 mmol, 0.1 eq) was added. The vial was closed with the lid, and the reaction was heated to 90° C. overnight. After cooling to room temperature the reaction was diluted with water/MTBE. The mixture was filtered through Celite, and the layers were separated. The organic layer was washed with water (×2), brine (×1), and dried over Na₂SO₄. The solids were filtered off, and the volatiles were removed via rotary evaporation. The residue was dissolved in DCM and silica gel (4 g) was added. The volatiles were removed via rotary evaporation. Purification via flash chromatography on silica gel eluting with 0-15% EtOAc in hexanes yielded 0.2962 g (1.17 mmol, 82% yield) of 416 as a yellow oil.

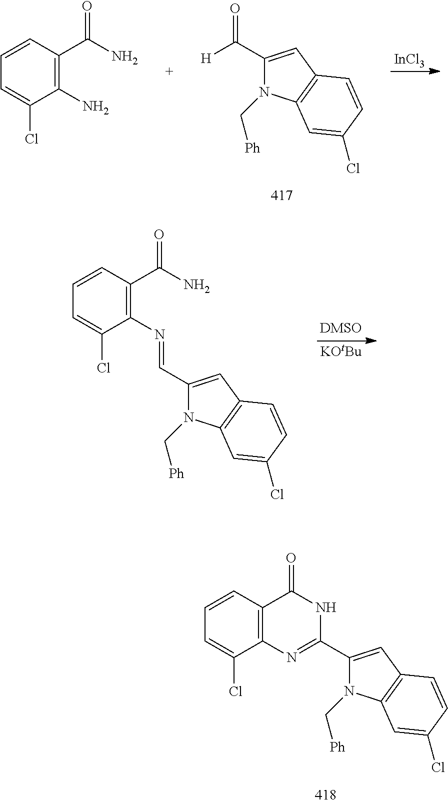 [SMILES: NC(=O)c1cccc(Cl)c1N ; O=Cc1cc2ccc(Cl)cc2n1Cc1ccccc1 ; "InCl3" ; NC(=O)c1cccc(Cl)c1/N=C/c1cc2ccc(Cl)cc2n1Cc1ccccc1 ; O=c1[nH]c(-c2cc3ccc(Cl)cc3n2Cc2ccccc2)nc2c(Cl)cccc12 ; "KOtBu"]

417 was prepared in an analogous manner as 150. A 50 mL round bottom flask was charged with 2-amino-3-chlorobenzamide (0.0504 g, 0.2965 mmol, 1 eq), 417 (0.080 mg, 0.2965 mmol, 1 eq) and a stir bar. The flask was evacuated and back-filled with N₂ (3×). Anhydrous CH₃CN (4 mL) was added with stirring. After 15 min, InCl₃ (0.019 g, 0.089 mmol, 0.3 eq) was added. The reaction was stirred at room temperature for 30 min before heating to 50° C. for 30 min, then left at room temperature overnight. The slurry was diluted with water. The solids (imine) were collected via vacuum filtration. The solids were transferred to a 20 mL sealed tube and dried under vacuum (0.085 g, 0.26 mmol, yield 68%), DMSO (1.6 mL) and a stir bar were added. Then potassium tert-butoxide (0.017 g, 0.1515 mmol, 1.0 eq.) was added. The vial was sealed, and the reaction mixture was heated to 100-120° C. overnight. After completion of the reaction the mixture diluted with water, solids were collected via vacuum filtration. Upon drying 0.07 g (overall yield of 56.17% from two steps) of 418 was collected. This material was used without purification.

The following benzamides were prepared in an analogous manner as 135,

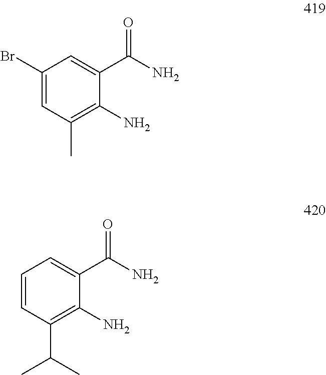

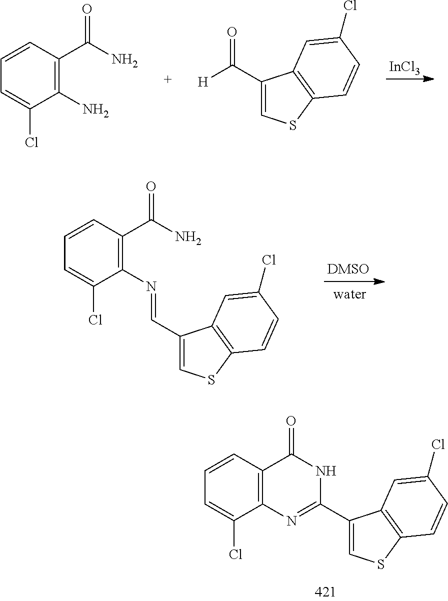

421 was prepared in an analogous manner as 176.

Using 415, 416, 419, and 420, the following compounds were prepared in an analogous manner as 199.

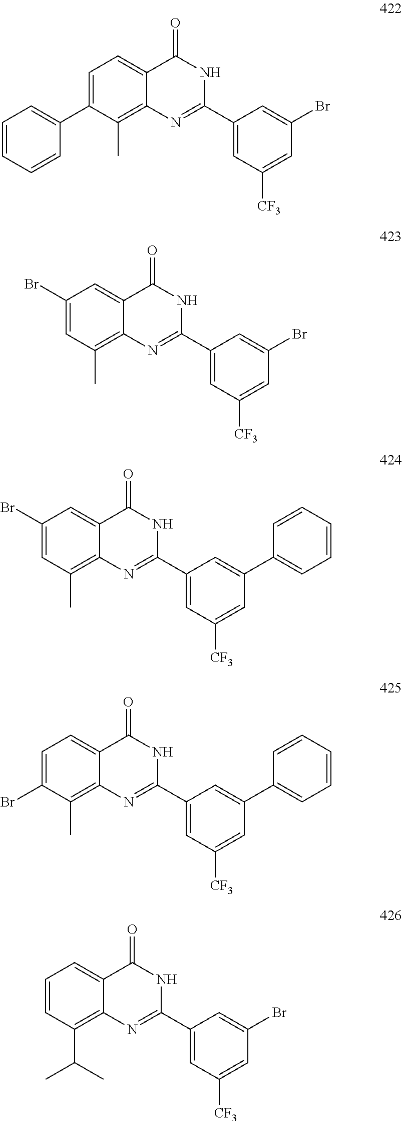 [SMILES: Cc1c(-c2ccccc2)ccc2c(=O)[nH]c(-c3cc(Br)cc(C(F)(F)F)c3)nc12 ; Cc1cc(Br)cc2c(=O)[nH]c(-c3cc(Br)cc(C(F)(F)F)c3)nc12 ; Cc1cc(Br)cc2c(=O)[nH]c(-c3cc(-c4ccccc4)cc(C(F)(F)F)c3)nc12 ; Cc1c(Br)ccc2c(=O)[nH]c(-c3cc(-c4ccccc4)cc(C(F)(F)F)c3)nc12 ; CC(C)c1cccc2c(=O)[nH]c(-c3cc(Br)cc(C(F)(F)F)c3)nc12]

The following compounds were prepared in an analogous manner as 219.

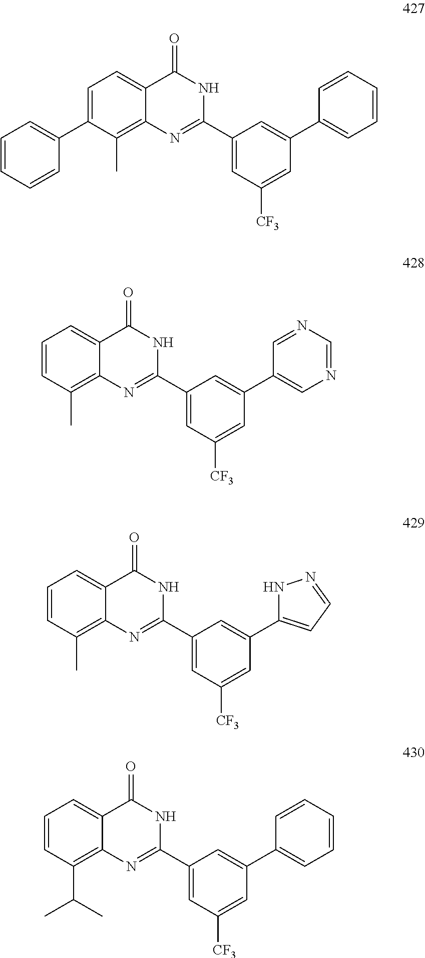 [SMILES: Cc1c(-c2ccccc2)ccc2c(=O)[nH]c(-c3cc(-c4ccccc4)cc(C(F)(F)F)c3)nc12 ; Cc1cccc2c(=O)[nH]c(-c3cc(-c4cncnc4)cc(C(F)(F)F)c3)nc12 ; Cc1cccc2c(=O)[nH]c(-c3cc(-c4ccn[nH]4)cc(C(F)(F)F)c3)nc12 ; CC(C)c1cccc2c(=O)[nH]c(-c3cc(-c4ccccc4)cc(C(F)(F)F)c3)nc12]

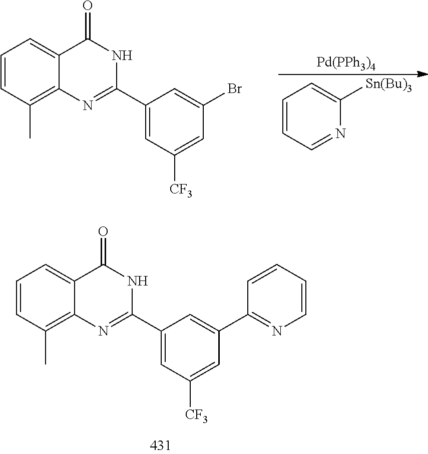

A 20 mL screw-top vial was charged with 2-(3-bromo-5-(trifluoromethyl)phenyl)-8-methylquinazolin-4(3H)-one (0.040 g, 0.10 mmol, 1 eq), 2-(tributylstannyl)pyridine (Combi-Blocks, 0.0576 g, 0.16 mmol, 1.5 eq), and a stir bar. The vial was evacuated and back-filled with Ar (×3). Anhydrous 1,4-dioxane (4 mL) was added. The vial was sealed with a septum, and the resulting stirred mixture was sparged with Ar (10 min). Pd(PPh₃)₄(0.0121 g, 0.01 mmol, 0.1 eq) was added. The vial was closed with the lid. The reaction was stirred at 90° C. over the weekend. After cooling to room temperature the reaction was diluted with water. The solids were collected via vacuum filtration. The filter cake was triturated with water (×3), 5% DCM in hexanes (×3), and hexanes (×1). After drying 0.026 g (0.068 mmol, 65% yield) of 431 was collected as an ash colored solid. Mass spectrum (ESI⁺): m/z=382 [M+1]observed.

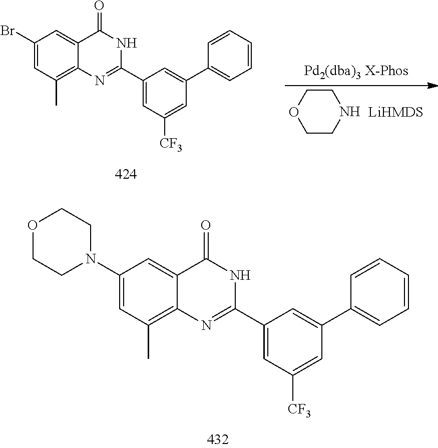

A 25 mL round bottom was charged with 10 (0.040 g, 0.087 mmol, 1 eq), Pd₂(dba)₃ (0.0080 g, 0.009 mmol, 0.1 eq), X-Phos (0.0083 g, 0.017 mmol, 0.2 eq) and a stir bar. The flask was evacuated and back-filled with Ar (×3). Anhydrous THF (4 mL) was added. The resulting dark purple solution was treated with morpholine (11.4 μL, 0.0114 g, 0.13 mmol, 1.5 eq) with stirring. After 5 min LiHMDS (1.0 M in THF, 0.44 mL, 0.44 mmol, 5 eq) was added dropwise to the resulting brown mixture. After 1 h the reaction was cooled to 0° C. and quenched with water. The solution was adjusted to pH≈7 with 1N HCl. The solids were collected via vacuum filtration. The filter cake was triturated with water (×3), 10% DCM in hexanes (×3), hexanes (×1), and dried. 0.0282 g (0.061 mmol, 70% yield) of 432 was isolated as a pale green solid. Mass spectrum (ESI⁺): m/z=466 [M+1]observed.

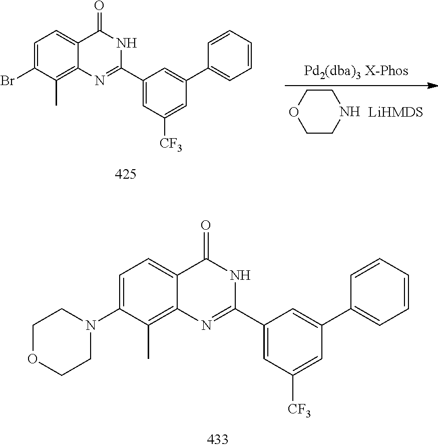

433 was prepared in an analogous manner as 432.

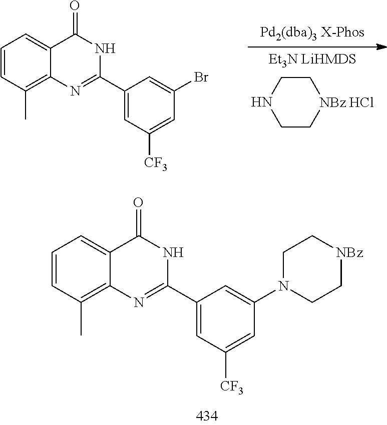

A 25 mL round bottom flask was charged with 2-(3-bromo-5-(trifluoromethyl)phenyl)-8-methylquinazolin-4(3H)-one (0.0501 g, 0.13 mmol, 1 eq), Pd₂(dba)₃ (0.0120 g, 0.013 mmol, 0.1 eq), X-Phos (0.0125 g, 0.026 mmol, 0.2 eq) and a stir bar. The flask was evacuated and back-filled with Ar (3×). Anhydrous THF (4 mL) was added. After stirring for 5 min the resulting purple solution was treated with 1-benzylpiperazine hydrochloride (Matrix, 0.0417 g, 0.2 mmol, 1.5 eq) and Et₃N (27 μL, 0.0198 g, 0.2 mmol, 1.5 eq). LiHMDS (1.0 M in THF, 0.78 mL, 0.78 mmol, 6 eq) was added 10 min later. After 1 h the volatiles were removed via rotary evaporation. The residue was diluted with water and adjusted to pH≈7 with 1N HCl. The solids were collected via vacuum filtration. The filter cake was triturated with water (3×), 10% DCM in hexanes (3×), hexanes (1×), and dried. 0.062 g (0.13 mmol, 100% yield) of 434 was isolated as a gray solid. Mass spectrum (ESI⁺): m/z=493 [M+1]observed.

The following compounds were prepared in an analogous manner as 256. 1,3-Diaminopropane was purchased and used without purification.

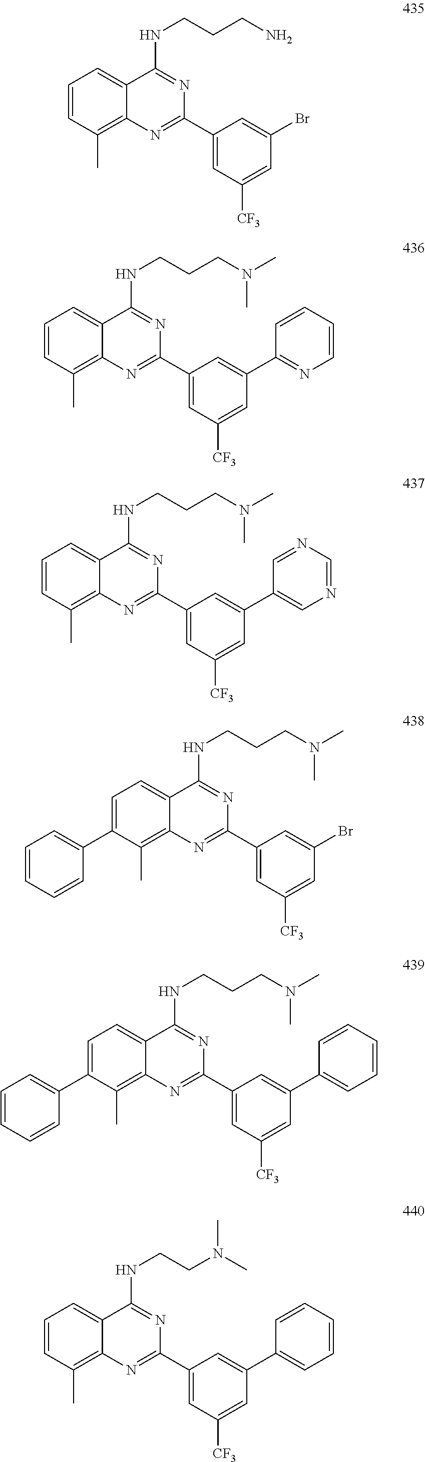

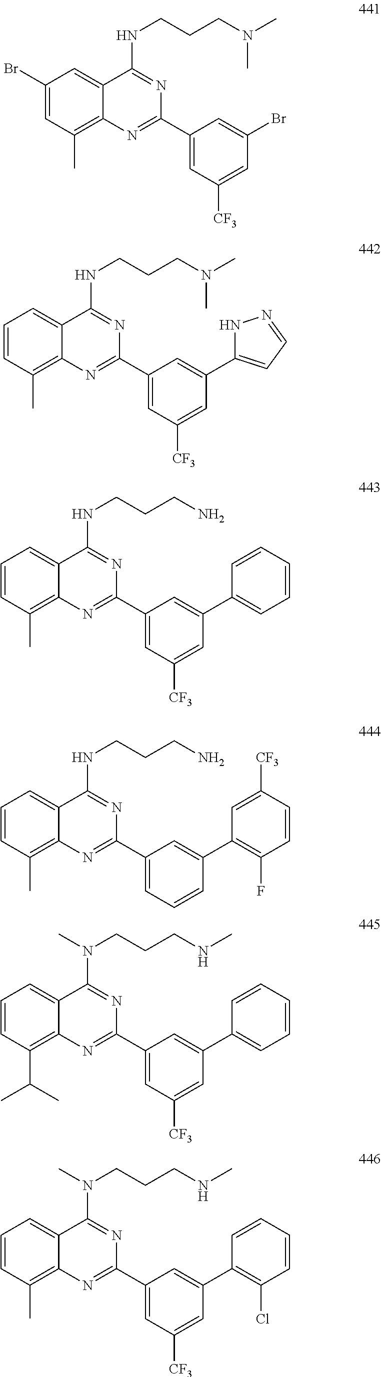

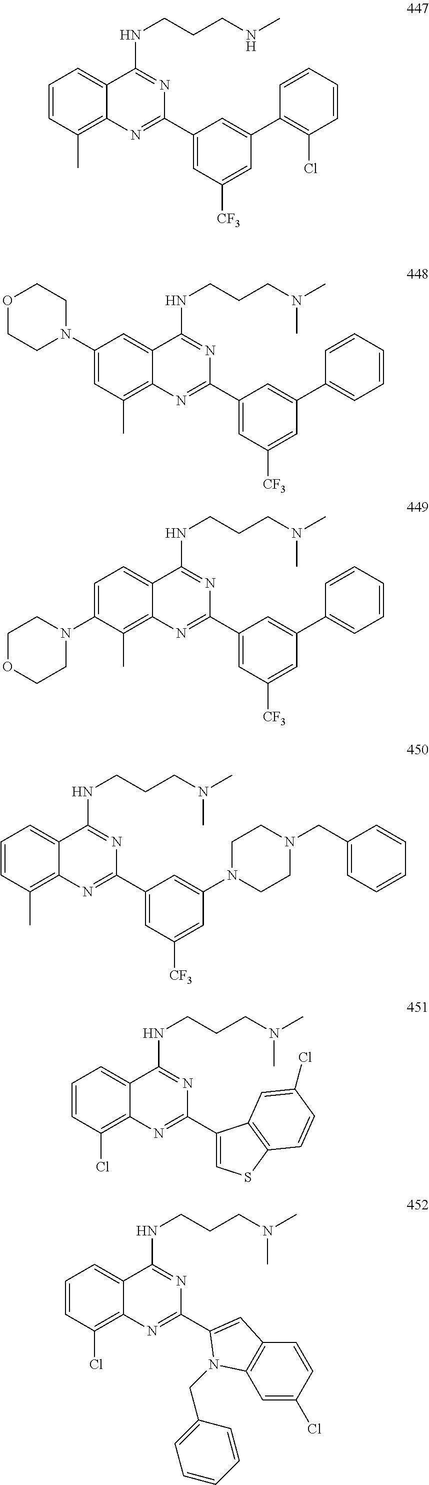

The following compounds were prepared in an analogous manner as 380. Tert-butyl N-[3-(methylamino)propyl]carbamate hydrobromide was purchased and used without purification.

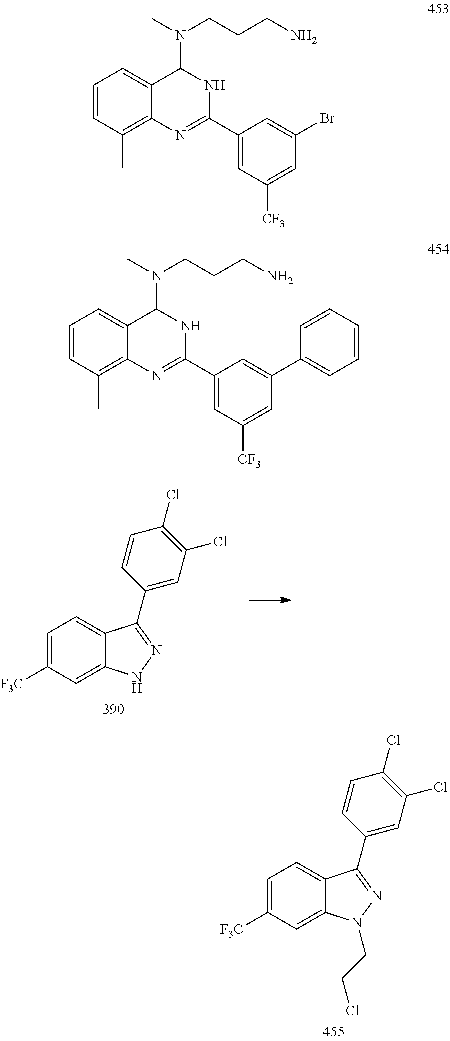 [SMILES: Cc1cccc2c1N=C(c1cc(Br)cc(C(F)(F)F)c1)NC2N(C)CCCN ; Cc1cccc2c1N=C(c1cc(-c3ccccc3)cc(C(F)(F)F)c1)NC2N(C)CCCN ; FC(F)(F)c1ccc2c(-c3ccc(Cl)c(Cl)c3)n[nH]c2c1 ; FC(F)(F)c1ccc2c(-c3ccc(Cl)c(Cl)c3)nn(CCCl)c2c1]

To 390 (60 mg, 0.18 mmol) in DMSO (3 ml), potassium tert-butoxide (81 mg, 0.72 mmol) was added followed by 2-chloro-N,N-dimethylethan-1-amine (52 mg, 0.36 mmol). After stirring at 80° C. for 2 d, the reaction mixture was cooled to room temperature. Then reaction mixture was quenched with water and extracted with ethyl acetate. The organic layer was washed with water, brine and dried. The crude residue was purified by column chromatography to yield 455 in 10% yield. Mass spectrum (ESI⁺): m/z=393 [M+1].

### 2-(dimethylamino)ethyl 5-chloro-3-(3,4-dichlorophenyl)-1H-indole-1-carboxylate

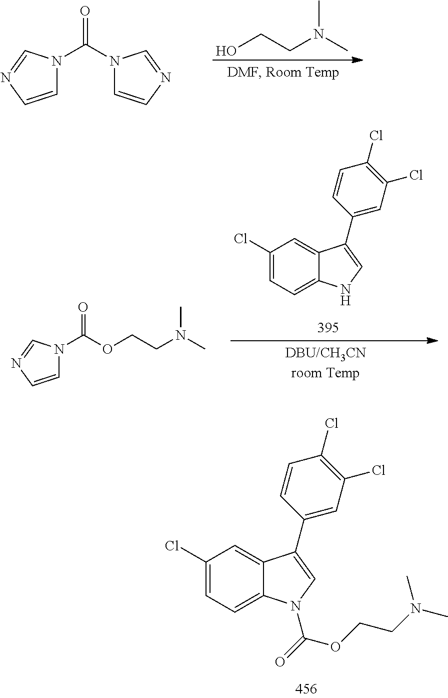

Under an atmosphere of Ar (g) in a 40 ml screw-top vial was placed carbodiimidazole (1.14 g, 9 mmol, 1 eq) and anhydrous dichloromethane (20 ml). The reaction mixture was stirred at 0° C. Dimethylaminoethanol (0.604 ml, 6 mmol, 0.66 eq) was added dropwise. The reaction was stirred at 0° C. for 1 hour and then overnight at room temperature. DCM (200 mL) was added. The mixture was washed with water (2×50 ml) and the layers were separated. The organic layer was dried (Na₂SO₄), filtered, and concentrated to give the imidazole intermediate as a clear oil. 395 (200 mg, 0.674 mmol, 1 eq) was placed in a 40 ml screw-top vial with stir bar and dissolved in dry CH₃CN (3 ml). The imidazole intermediate was added at room temperature followed by the addition of DBU (20 mg, 0.1349 mmol, 0.2 eq). The reaction was stirred at room temperature for 4 h and then the solvent was removed in vacuuo to give a residue. The residue as partitioned with EtOAc and water, and dried (Na₂SO₄). The organic layer was filtered and concentrated to give a white solid. Purification via silica gel chromatography (DCM to 95/5 DCM/MeOH) provided the pure carbamate product 456. ¹H NMR (DMSO-d₆, 400 MHz): δ 8.18-8.24 (m, 2H), 7.99 (s, 1H), 7.86 (d, 1H), 7.74 (m, 2H), 7.48 (dd, 1H), 4.52 (t, 2H), 2.71 (t, 2H), 2.24 (s, 6H).

### E-1-(5-bromo-3-(3,4-dichlorostyryl)-1H-indol-1-yl)-2-(dimethylamino)ethan-1-one

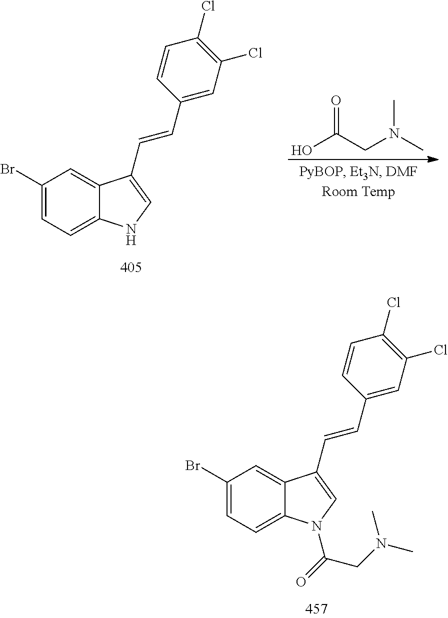

In a 40 ml screw-top vial under Ar (g) with a stirbar, was added DMF (5 mL), 405 (500 mg, 1.36 mmol, 1 eq), dimethylglycine (224.8 mg, 2.18 mmol, 1.6 eq), PyBOP (1.063 g, 2.043 mmol, 1.5 eq) and Et₃N (275.6 mg, 0.38 ml, 2.724 mmol, 2 eq). The reaction mixture was stirred overnight at room temperature, and then poured into 300 ml of water/brine before extraction with EtOAc (3×200 ml). The organic layer was dried (Na₂SO₄), filtered, and concentrated to give a white crude solid. Purification with silica gel chromatography (1:1 hexanes/EtOAc) provided 457. ¹H NMR (DMSO-d₆, 400 MHz): δ 8.37 (s, 1H), 8.32 (d, 1H), 8.29 (s, 1H), 8.03 (s, 1H), 7.54-7.68 (m, 4H), 7.33 (d, 1H), 3.84 (s, 2H), 2.37 (s, 6H).

### 1-(5-chloro-3-(3,4-dichlorophenyl)-1H-indol-11yl)-2-(2-methoxyethoxy)ethan-1-one

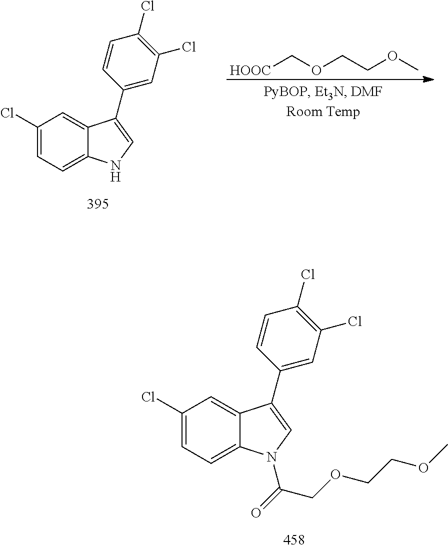

In a 40 ml screw-top vial under Ar (g) with a stirbar, was added DMF (3 mL), 395 (500 mg, 1.686 mol, 1 eq), 2-(2-methoxyethoxy)acetic acid (362 mg, 2.698 mmol, 1.6 eq), PyBOP (1.316 grams, 2.529 mmol, 1.5 eq, Aldrich), and Et₃N (341.2 mg, 0.47 ml, 3.372 mmol, 2 eq). The reaction mixture was stirred overnight at room temperature and then poured into 300 ml water before extraction with EtOAc. Concentration provided the crude product. Silica gel chromatography (80/20 hexanes/ethyl acetate) provided 458. ¹H NMR (DMSO-d₆, 400 MHz): δ 8.41 (d, 1H), 8.28 (s, 1H), 7.95 (s, 1H), 7.83 (d, 1H), 7.73 (s, 2H), 7.41 (dd, 1H), 4.90 (s, 2H), 3.76 (m, 2H), 3.55 9 m, 2H), 3.28 (s, 3H).

### (S)-2-amino-1-(5-chloro-3-(3,4-dichlorophenyl)-1H-indol-1-yl)butan-1-one methanesulfonate salt

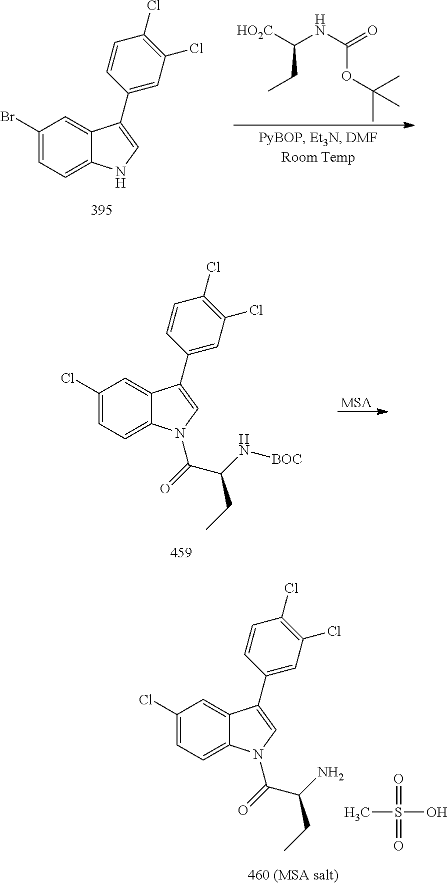

In a 40 ml screw-top vial under Ar (g), was added 395 (500 mg, 1.686 mmol, 1 eq), BOC-L-aminobutyric acid (549 mg, 2.7 mmol, 1.6 eq), PyBOP (1.316 grams, 2.529 mmol, 1.5 eq, Aldrich), and Et₃N (341.2 mg, 0.47 ml, 3.372 mmol, 2 eq). The reaction mixture was stirred overnight at room temperature and then poured into 300 ml water/brine before extraction with EtOAc (3×200 ml). The organic layer was dried (Na₂SO₄), filtered and concentrated to remove the solvent to give crude 459. Silica gel chromatography (80/20 hexanes/EtOAc) provided pure 459.

To 459 (260 mg, 0.5417 mmol, 1 eq) in a 40 ml screw-top vial was added MeOH (30 ml) and methanesulfonic acid (0.11 ml, 156.2 mg, 3 eq). The reaction mixture was stirred overnight at room temperature. The solvent was removed in vacuuo to give a residue which was washed with a small amount of EtOAc to afford 460 as the MSA salt. 1H NMR (DMSO-d₆, 400 MHz): δ 8.59 (s, 1H), 8.52 (brs, 3H), 8.47 (d, 1H), 8.04 (d, 1H), 7.94 (d, 1H), 7.78-7.84 (m, 2H), 7.56 (dd, 1H), 5.09 (bs, 1H), 2.31 (s, 3H, msa), 1.90-2.10 (m, 2H), 0.99 (t, 3H).

### 3,3′-(propane-2,2-diyl)bis(5,6-dichloro-1H-indole)

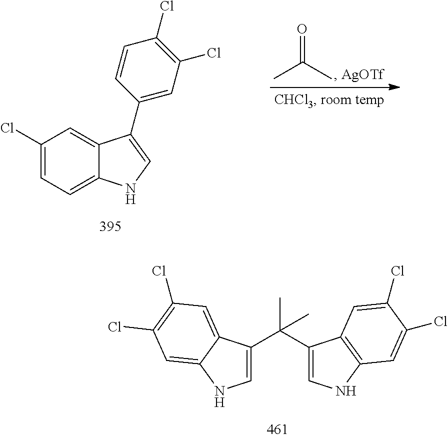

In a 40 ml screw-top vial under Ar (g) with a stir bar was added AgOTf (68.8 mg, 0.268 mmol, 5 mol %), 395 (1.0 grams, 5.375 mmol, 1 eq), acetone (0.395 ml, 5.375 mmol, 1 eq) and anhydrous chloroform (3 ml). This mixture was stirred overnight at room temperature. Then EtOAc was added, and the mixture was filtered through 1″ silica gel to remove silver solids. Evaporation of the solvent gave the crude material. Silica gel purification (90/10 hexanes/EtOAc) provided 461 as a white semi-solid. ¹H NMR (DMSO-d₆, 400 MHz): δ 11.19 (br s, 2H, NH), 7.56 (s, 2H), 7.52 (m, 1H), 7.14 (s, 2H), 1.77 (s, 6H).

### (E)-5-chloro-3-(,4-dichlorstyryl)-2-methyl-1H-indole

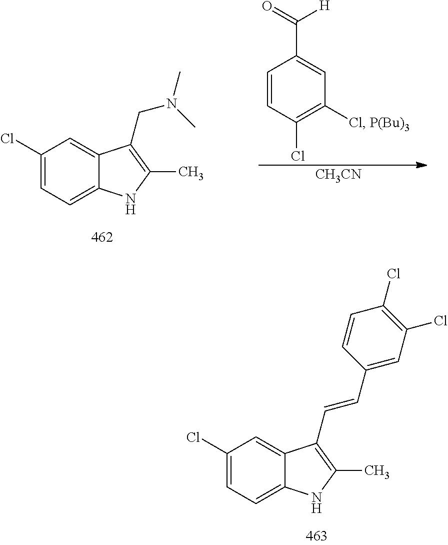

In a 250 ml round bottom flask flushed with Ar (g), was added 462 (which was made in the same manner as 129) (3.58 grams crude, 16.07 mmol, 1 eq) and CH₃CN (80 ml). 3,4-Dichlorobenzaldehyde (4.2 grams, 24 mmol, 1.5 eq) was then added followed by tri n-butylphosphine (4.86 grams, 6 ml, 24 mmol, 1.5 eq). This mixture was placed on a pre-heated oil bath (81° C.) and heated overnight. The solvent was removed in-vacuuo to give a residue which was dissolved in a minimum amount of EtOAc/hexanes and passed through a silica gel pad with gravity (90/10 hexanes/EtOAc to 1:1 hexanes/EtOAc) to give the crude product. Further purification with a silica gel column (80/20 hexanes/EtOAc to 70/30 hexanes/EtOAc) provided the pure product 463 as a white solid. ¹H NMR (DMSO-d₆, 400 MHz): δ 11.5 (br s, 1H, NH), 8.01 (dd, 2H), 7.68 (dd, 1H), 7.55 (d, 1H), 7.48 (d, 1H, trans H), 7.39 (d, 1H), 7.10 (dd, 1H), 6.07 (d, 1H, trans H), 2.56 (s, 3H).

### (E)-N¹-(2-(3,4-dichlorostyryl)-7-methoxy-8-methylquinazolin-4-yl)-N³,N³-dimethylpropane-1,3-diamine

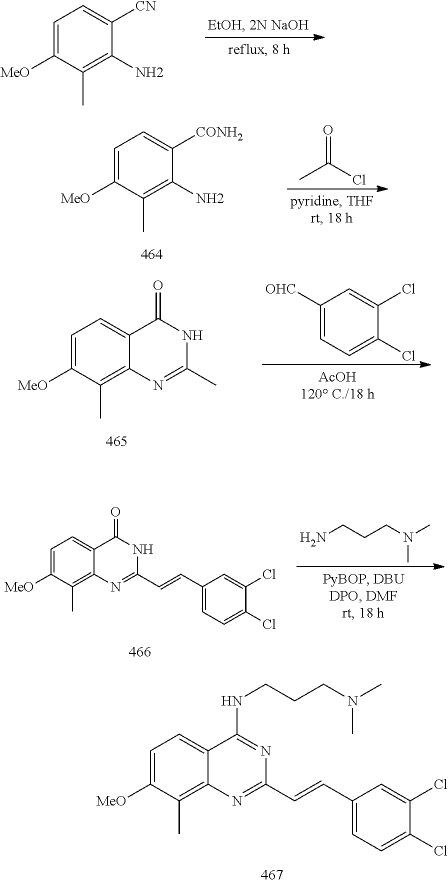

A 100 mL round bottom flask was charged with ethanol (6.4 mL) and a stir bar. 2-Amino-4-methoxy-3-methylbenzonitrile (400 mg, 2.46 mmol, 1 eq.) was added followed by 2 N NaOH solution (12.8 mL). The resulting mixture was heated to reflux for 8 h. The mixture was brought to room temperature, diluted with water (30 mL) and extracted with a EtOAc:THF (9:1) mixture (3×30 mL). The combined organic layer was dried over Na₂SO₄, filtered and evaporated to provide 320 mg of 464 (72%).

A 50 mL round bottom flask was charged with THF (7 mL) and stir bar. 464 (320 mg, 1.77 mmol, 1 eq.) and pyridine (0.186 mL, 1.3 eq.) were added, followed by acetyl chloride (0.28 mL, 3.90 mmol, 2.2 eq.). The resulting mixture was stirred overnight at room temperature. After completion of the reaction, the reaction mixture was concentrated under reduced pressure to give crude material, which was treated with 2N NaOH (30 mL) and filtered. The filtrate was neutralized with 6 N HCl, and solids were filtered and washed with water. Upon drying, 465 was obtained (190 mg, 52.39% yield). Purity 94.85% by LCMS, m/z: 205.2 [M+1].

A 25 mL round bottom flask was charged with acetic acid (1 mL) and stir bar. 465 (50 mg, 0.244 mmol, 1 eq.) and 3,4-dichlorobenzaldehyde (43 mg, 0.244 mmol, 1 eq.) were added. The resulting mixture was stirred overnight at 120° C. After completion of the reaction, the mixture was cooled to room temperature and diluted with methanol. Solids were filtered and dried to obtain 466 (65 mg, 73.8% yield).

A 50 mL round bottom flask with stir bar was charged with 466 (140 mg, 0.3885 mmol, 1 eq.). The flask was evacuated and back-filled with N₂ (3×). Anhydrous DMF (15 mL) was added followed by (benzotriazol-1-yloxy)tripyrrolidinophosphonium hexafluorophosphate (0.262 g, 0.504 mmol, 1.3 eq.). The resulting stirred suspension was treated with 1,8-diazabicyclo[5.4.0]undec-7-ene (0.087 mL, 0.582 mmol, 1.5 eq.) and diphenylether (0.0616 mL, 0.3885 mmol, 1 eq.). A homogeneous solution rapidly formed. After 10 min, N1,N1-dimethylpropane-1,3-diamine (0.0727 mL, 0.582 mmol, 1.5 eq) was added. The reaction mixture was stirred at room temperature for 16 h. After completion of the reaction, the mixture was partitioned between EtOAc/water, and the layers were separated. The organic layer was washed with water (2×), brine (1×), and dried over Na₂SO₄. The solids were filtered off, and the volatiles were removed via rotary evaporation. Purification by preparative TLC, eluting with 6% MeOH in DCM, yielded 0.20 mg (11.5% yield) of the desired product (467) as a sticky pale yellow semi solid. ¹H NMR (CDCl₃): δ 7.79 (m, 2H), 7.62 (s, 1H), 7.4 (m, 2H), 7.09 (m, 2H), 3.85 (s, 3H), 3.65 (t, 2H), 2.55 (m, 2H), 2.4 (s, 3H), 2.30 (s, 6H), 1.91 (m, 2H). Mass spectrum (ESI⁺): m/z=445.3 [M+1].

### Example 2

### Assay for RecA's ssDNA-Dependent ATPase Activity

A coupled enzyme ATPase assay, similar to that described in Wigle and Singleton, BioorgMed Chem Lett. 17(12):3249-53 (2007), was used to screen compounds for in vitro activity against RecA. A solution of the compound to be tested dissolved in DMSO (5 μL of 20× concentrated stock) was added to 90 μL of a master mix containing the following components at their final concentrations in the appropriate wells of flat-bottomed black 96-well plates (Corning Life Sciences, Corning N.Y.) at 37° C.: 0.5 μM E. coli RecA (New England BioLabs), 10 mM Mg(OAc)2 (Sigma, St. Louis, Mo.), 500 μM ATP (Sigma), 250 μM phosphoenolpyruvate (Sigma), 100 μM amplex red (Invitrogen, Carlsbad, Calif.), 1 U/mL pyruvate kinase (Sigma), 1 U/mL pyruvate oxidase (Thermo Fisher Scientific, Waltham, Mass.), 1 U/mL horseradish peroxidase (Sigma), and 20 mM Tris*HOAc, pH 7.5 (Boston BioProducts, Ashland, Mass.). The total volume of the assay solution was 100 μL. Exemplary compounds of the invention were tested at concentrations of 40 μM, 16 μM, 6.4 μM, 2.6 μM, 1.0 μM, 0.41 μM, 100 μM, prepared from serial dilutions of a stock solution of 100 μM compound.

The fluorescence emission at 595 nm (excitation at 485 nm) was then recorded using a microplate reader (PolarStar Optima, BMG Labtech, Ortenberg, Germany) for approximately 25 min to establish the baseline fluorescence.

To initiate the reactions, poly(dT) (5 μL of a 100 μM-nts stock solution) was added to each well to give a final DNA concentration of 5 μM-nts. Fluorescence emission was recorded for an additional 90 min. Positive control reactions lacking the test compound were included to measure the unimpeded RecA activity. Negative control reactions without RecA or without RecA and the poly(dT) were also included to mimic the complete inhibition of RecA-mediated ATP hydrolysis.

### Assay for Ciprofloxacin-Induced SOS Reporter Gene Expression

An SOS reporter gene assay was used to assess the ability of the RecA inhibitor compounds to block the SOS response to ciprofloxacin. Details of the testing methodology are provided in, for example, Sandler et al., Genetics 143(1):5-13 (1996) and McCool et al., Mol Microbiol. 53(5):1343-57 (2004). The SOS genes are regulated by the transcriptional repressor LexA and the DNA-binding protein RecA. When DNA damage occurs, RecA binds to ssDNA, and this protein-DNA complex then induces the autocleavage of LexA, resulting in the depression of LexA-bound promoters. The expression of the cell-division inhibitor SulA is highly up-regulated during the SOS response. Therefore, a reporter construct wherein the sulA promoter (sulAp) is fused to the lacZ gene can be used to measure the level of the SOS response in cells using an assay for β-galactosidase activity.

E. coli strain DM4000 contains the sulAp-lacZ reporter gene inserted at the X attachment site. Two additional strains containing the same sulAp-lacZ reporter gene were used as RecA-independent controls in this assay. JC19098 encodes an un-cleaveable LexA repressor; therefore, the sulA promoter-dependent β-galactosidase expression is always off. The complementary control strain SS1492 lacks LexA, and β-galactosidase expression is always on, independent of RecA's activity state. Additional controls used for each strain included wells lacking ciprofloxacin (no SOS response) and wells lacking ciprofloxacin and compound (uninhibited SOS response).

On the day prior to performing the SOS assay, E. coli cells were inoculated into 3 mL of LB broth and grown overnight at 37° C. in an orbital shaker. On the day of the assay, the overnight culture was inoculated into fresh LB at a 1:100 dilution and grown to an OD₆₀₀>0.5. After reaching an appropriate density, the cells were diluted to OD₆₀₀=0.01. If used in the experiment, Polymyxin B nonapeptide (PMBN; Sigma) was added to the cells to a final concentration of 4 μg/mL. Then, 46 μL of diluted cells were added to the wells of a sterile white round-bottom 96-well plate (Corning Life Sciences). Compounds diluted in DMSO (2 μL of a 25× concentrated stock solution) were added to the cells to yield final concentrations of 100, 33, 11, and 3.7 M. The plates were sealed with a breathable sterile film (Sigma) and incubated on a shaker at room temperature for 20 min.

Following the 20-min pre-incubation period, ciprofloxacin (2 μL; Hospira, Inc., Lake Forest, Ill.) was added to the cells at a final concentration of 20 ng/mL to induce the SOS response. The plates were again sealed with a breathable sterile film and incubated at 37° C. with shaking for 1 h. After this incubation period, lysozyme (2 μL of an 8 mg/mL solution; final concentration of approximately 0.3 mg/mL) was added to each well, and the plates were incubated at room temperature on a shaker for 20 min to allow cell lysis. Beta-Glo reagent (30 μL; Promega; Madison, Wis.) was then added to each well, and the plate was incubated for an additional 40 min at room temperature (10 min with shaking, 20 min still, 10 min with shaking).

Luminescence was measured on a plate reader with the gain adjustment set to 40% of the maximum on a well with one of the highest luminescence values on the plate (e.g., a well containing ciprofloxacin without inhibitor). For assays performed both with and without PMBN, the gain was set separately for each condition.

### Assay for Shifts in the Minimum Inhibitory Concentration (MIC) of Antibiotics

Minimum inhibitory concentration (MIC) shift assays were performed to determine if the test compounds could alter the MIC of existing antibacterial drugs (e.g., ciprofloxacin and colistin) for E. coli, Acinetobacter baumannii, Klebsiella pneumoniae, Pseudomonas aeruginosa, and Enterobacter cloacae. The MIC shift assay is based on the standard broth microdilution MIC determination protocol of Wiegand et al, Nat Protoc. 3(2):163-75 (2008), with slight modifications that allow for the screening of up to seven test compounds in a single plate.

On the day before an assay, bacteria were grown overnight in cation-adjusted Mueller Hinton broth (CAMHB; Teknova, Hollister, Calif.) at 37° C. in an orbital shaker and sub-cultured at a 1:200 dilution the following morning. The cells were grown to OD₆₀₀=0.5 to 0.8 and then diluted to OD₆₀₀=0.01. From a serial dilution of each antibiotic at (50 μL of 2× concentrated stock solutions) several concentrations were added to the wells of a sterile, transparent round-bottom 96-well plate (Phenix Research Products, Candler, N.C.), and diluted cells (50 μL) were added to the plates for a starting OD₆₀₀ of 0.005. Then, a test compound (2.5 μL) was added to each well to give a final concentration of 4 or 10 μM. A row of wells lacking test compound but containing the antibiotic was used to determine the MIC of that antibiotic alone. A column of wells lacking both antibiotic and test compound served as a growth control, and an additional column containing only CAMHB was included as a sterile blank control.

The plates were incubated for 18 to 20 h at 37° C. in an orbital shaker, and the cell density (OD₆₀₀) was determined the following day in a microplate reader (PolarStar Optima, BMG Labtech). The MIC shift was determined by comparing the MIC of the antibiotic alone to the MIC of the antibiotic plus test compound.

### Assay for Potentiation of Antibacterial Killing

An antibiotic potentiation assay was used to evaluate the effects of different concentrations of test compound on the sensitivity of bacterial strains to varying concentrations of antibiotics. It is carried out in same manner as an assay for a shift in minimum inhibitory concentration (MIC), but with varying concentrations of inhibitor and antibiotic.

Briefly, each member of a two-fold dilution series (50 μL of 2× concentrated stock solutions) of an antibiotic (e.g., ciprofloxacin or colistin) in CAMHB was added to a microplate with the highest concentration in the left column of the plate. Mid-log phase cells were diluted to OD₆₀₀=0.01 in CAMBH, and 50 μL of each cell diluted cell suspension were added to each well, with the exception of the sterile blank control. Each member of a two-fold serial dilution of the test compound (2.5 μL of 50× concentrated stocks solutions) were added to the wells so that the top row of the plate contained the highest concentration of inhibitor. The inhibitor concentration decreased down the plate, and the last row contained only vehicle (DMSO). The plates were incubated for 18 to 20 h at 37° C. in an orbital shaker, and the cell density (OD₆₀₀) was determined the following day in a microplate reader (PolarStar Optima, BMG Labtech).

Using the above described in vitro methods, exemplary compounds of the invention were tested for their activity. Select results from the tests are shown in Tables 16-19.

TABLE 16

| | | | Colistin MIC | |
|---|---|---|---|---|
| | | | shift (Compound | |
| | | | concentration: 10 μM) | |
| | | | Acinetobacter | E.coli |
| | | | baumannii | (SCR1) |
| | | | (Ab23) (MIC | (MIC of |
| | | | of colistin | colistin |
| | RecA | SOS | alone: | alone: |
| | Inhibition | Attenuation | >8.0 | 0.5-1.0 |
| Compound ID | IC₅₀ (μM) | EC₅₀ (μM)* | μg/mL) | μg/mL) |
| 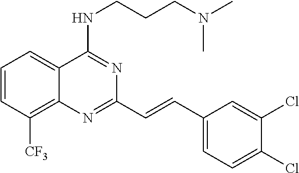 [SMILES: CN(C)CCCNc1nc(/C=C/c2ccc(Cl)c(Cl)c2)nc2c(C(F)(F)F)cccc12] MLJ-02-070A | 12 | <6 uM | >128X | 16-32X |
| 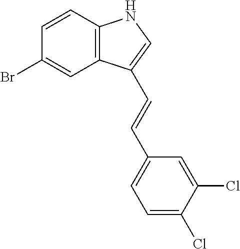 [SMILES: Clc1ccc(/C=C/c2c[nH]c3ccc(Br)cc23)cc1Cl] MLJ-02-086B | 15 | 8 uM | >64X | 8-16X |

*SOS attenuation EC₅₀ in the presence of PMBN (4 μg/mL)

TABLE 17

| | Colistin MIC Shift (compd. concentration: 4.0 μM) | |
|---|---|---|
| | A. baumannii (MDR Ab) | E. coli (SCR1) |
| Compound No.* | (MIC: 0.5-1.0 μg/mL) | (MIC: 0.5-1.0 μg/mL) |
| 353 | 8x | 8x |
| 492 | 8x | 8x |
| 343 | 8x | 16x |
| 342 | 8x | 8x |
| 339 | 8x | 8x |
| 337 | 4x | 4x |
| 338 | 4x | 4x |
| 505 | 4x | 8x |
| 506 | 4x | 8x |
| 487 | 8x | 16x |
| 345 | 4x | 16x |
| 348 | 8x | 16x |
| 361 | 4x | 4x |
| 360 | 4x | 4x |
| 429 | 2x | 4x |

*For corresponding chemical structure, see Tables 2-15.

TABLE 18

| | MIC Shift (Compd. No. 429* |
|---|---|
| Antibiotic | Concentration: 8 μM)** |
| Meropenem | ≧16x |
| Tobramycin | ≧16x |
| Ciprofloxacin | ≧32x |
| Levofloxacin | ≧64x |

*For corresponding chemical structure, see Table 5.
**Tested againstA. baumannii (Ab23)

TABLE 19

| | | | | Colistin with compound* (at 10 μM) or compound* alone (at 10 μM) | | | | |
|---|---|---|---|---|---|---|---|---|
| | | | | | Colistin & | | Colistin & | |
| | MMX or | Culture | | Cmpd No. | Cmpd No. 429 | Cmpd. No. | Cmpd No. 343 | |
| Isolate | ATCC No. | Type | Colistin | 429 alone^{2,3} | MIC shift | 343 alone^{2,4} | MIC shift | Polymyxin B |
| Escherichia | 25922 | QC; | 0.12 | 0.015, >32 | 8 | 0.015, >32 | 8 | 0.12 |
| coli | 102 | Colistin-S | (0.25-2)¹ | | | | | (0.25-2)¹ |
| Pseudomonas | 27853 | QC; | 0.25 | 0.12, >32 | 2 | 0.06, >32 | 4 | 0.25 |
| aeruginosa | 103 | Colistin-S | (0.5-4)¹ | | | | | (0.5-2)¹ |
| Pseudomonas | 1582 | Colistin-I | 4 | 0.25, >32 | 16 | 0.5, >32 | 2 | 4 |
| aeruginosa | | | | | | | | |
| Pseudomonas | 6977 | Colistin-R | >64 | 1, >32 | 64 | 1, >32 | 64 | 64 |
| aeruginosa | | | | | | | | |
| Klebsiella | 13883 | Colistin-S | 0.12 | 0.03, >32 | 4 | 0.015, >32 | 8 | 0.5 |
| pneumoniae | 214 | | | | | | | |
| Klebsiella | 2237 | Colistin-R | 32 | 0.25, >32 | 128 | 0.25, >32 | 128 | 32 |
| pneumoniae | | | | | | | | |
| Acinetobacter | 19606 | Colistin-S | 0.25 | 0.03, >32 | 8 | 0.015, 16 | 17 | 0.25 |
| baumannii | 1630 | | | | | | | |
| Acinetobacter | Ab23 | Colistin-R | 64 | 0.03, >32 | 2133 | 0.015, 8 | 4267 | 64 |
| baumannii | | | | | | | | |
| Serratia | 6147 | Colistin-R | >64 | 0.25, >32 | 256 | 0.25, >32 | 256 | >64 |
| marcescens | | | | | | | | |
| Providencia | 6644 | Colistin-R | >64 | 16, >32 | 4 | >16, >32 | 4 | >64 |
| stuartii | | | | | | | | |
| Proteus | 6240 | Colistin-R | >64 | 0.06, >32 | 1067 | >16, >32 | 4 | >64 |
| vulgaris | | | | | | | | |
| Burkholderia | 547 | Colistin-R | >64 | >16, >32 | 4 | >16, >32 | 4 | >64 |
| cepacia | | | | | | | | |
| Staphylococcus | 43300 | MRSA | >64 | ≦0.015, 2 | 4267 | ≦0.015, 1 | 4267 | 64 |
| aureus | 5999 | | | | | | | |
| Staphylococcus | 29213 | MRSA | >64 | ≦0.015, 2 | 4267 | ≦0.015, 1 | 4267 | 645 |
| aureus | 100 | | | | | | | |

*Corresponding chemical structure shown in Tables 2-15.
¹CLSI QC range shown in parentheses.
²Precipitation at drug concentration 64 μg/mL in MHB II medium.
³Tested in combination with colistin at 3 μg/mL
⁴Tested in combination with colistin at 5 μg/mL

In addition, the following compounds demonstrated activity as colistin potentiators.

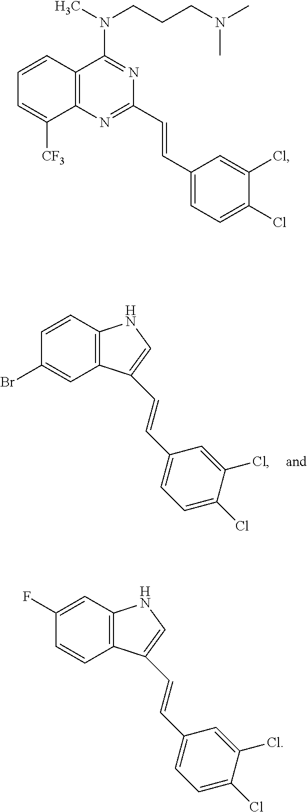 [SMILES: CN(C)CCCN(C)c1nc(/C=C/c2ccc(Cl)c(Cl)c2)nc2c(C(F)(F)F)cccc12 ; Clc1ccc(/C=C/c2c[nH]c3ccc(Br)cc23)cc1Cl ; "and" ; Fc1ccc2c(/C=C/c3ccc(Cl)c(Cl)c3)c[nH]c2c1]

The following compounds demonstrated activity as potent SOS inhibitors.

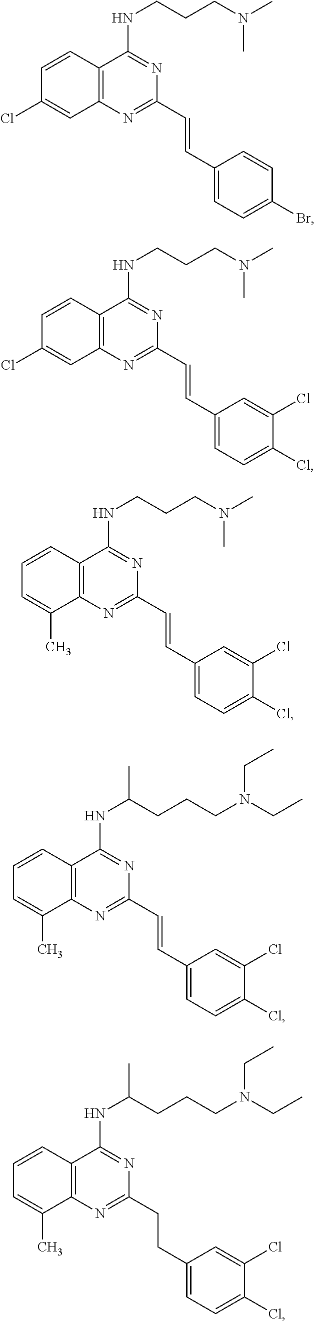

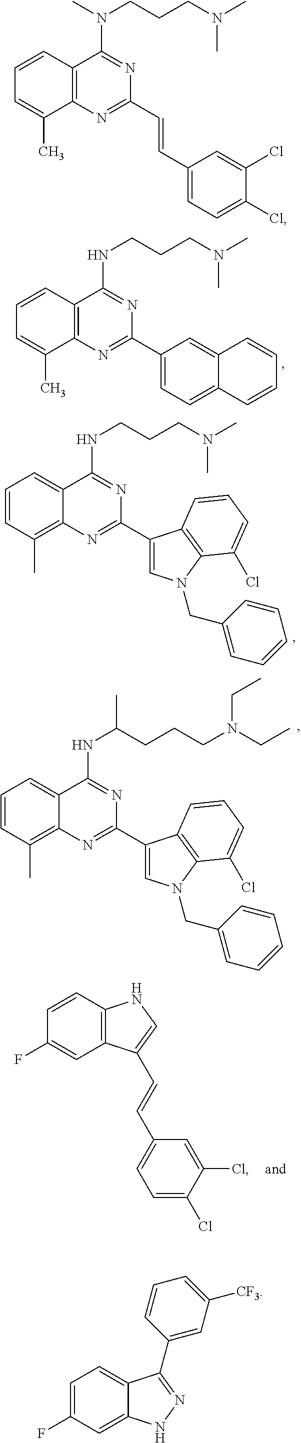

Exemplary data is shown in Tables 20-22. See Tables 2-15 for the corresponding compound chemical structures.

TABLE 20

| | colistin MIC shift, Ab23 | |
|---|---|---|
| No. | at (compd concentration) | |
| 301 | 8x | (8 μM) |
| 302 | 16x | (4 μM) |
| 303 | nc* | (4 μM) |
| 304 | nc | (4 μM) |
| 305 | 4x | (4 μM) |
| 306 | 4x | (8 μM) |
| 307 | 4x | (8 μM) |
| 308 | nc | (8 μM) |
| 309 | 4x | (10 μM) |
| 310 | 8x | (10 μM) |
| 311 | nc | (10 μM) |
| 312 | 16x | (4 μM) |
| 313 | 8x | (4 μM) |
| 314 | 8x | (4 μM) |
| 315 | 4-8x | (4 μM) |
| 316 | 2-8x | (4 μM) |
| 317 | 2x | (4 μM) |
| 318 | 2x | (4 μM) |
| 319 | 2x | (4 μM) |
| 320 | 2x | (4 μM) |
| 321 | 8x | (10 μM) |
| 322 | nc | (4 μM) |
| 323 | nc | (4 μM) |
| 324 | nc | (4 μM) |
| 325 | 4x | (4 μM) |
| 326 | nc | (4 μM) |
| 327 | 8-16x | (4 μM) |
| 328 | nc | (4 μM) |
| 329 | 8x | (4 μM) |
| 330 | 2x | (4 μM) |
| 331 | 16x | (4 μM) |
| 332 | 16x | (4 μM) |
| 333 | nc | (4 μM) |
| 334 | nc | (4 μM) |
| 335 | 16x | (4 μM) |
| 336 | 8-16x | (2 μM) |
| 337 | 8-16x | (2 μM) |
| 338 | 8-16x | (2 μM) |
| 339 | 8-16x | (2 μM) |
| 340 | 8-16x | (2 μM) |
| 341 | 8-16x | (2 μM) |
| 342 | 8-16x | (2 μM) |
| 343 | 8-16x | (2 μM) |
| 344 | 8-16x | (2 μM) |
| 345 | 8-16x | (2 μM) |
| 346 | 8-16x | (2 μM) |
| 347 | 8-16x | (2 μM) |
| 348 | 8-16x | (2 μM) |
| 349 | 8-16x | (2 μM) |
| 350 | 8-16x | (2 μM) |
| 351 | 8-16x | (2 μM) |
| 352 | 8-16x | (2 μM) |
| 353 | 8-16x | (2 μM) |
| 354 | 8-16x | (2 μM) |
| 355 | 8-16x | (2 μM) |
| 356 | 16-64x | (4 μM) |
| 357 | 4-8x | (4 μM) |
| 358 | nc | (8 μM) |
| 359 | 16x | (4 μM) |
| 360 | 16x | (2 μM) |
| 361 | 16x | (2 μM) |
| 362 | 16x | (2 μM) |
| 363 | 32-64x (4 μM); | |
| | ≧16x (2 μM) | |
| 364 | 32-64x | (4 μM) |
| 365 | 16-64x (4 μM); | |
| | ≧16x (2 μM) | |
| 366 | 16-32x | (4 μM) |
| 367 | 16-32x | (4 μM) |
| 368 | 16-32x | (4 μM) |
| 369 | 32x | (4 μM) |
| 370 | 32x | (4 μM) |
| 371 | 32x | (4 μM) |
| 372 | 8-32x | (4 μM) |
| 373 | 8-16x | (4 μM) |
| 374 | 16x | (4 μM) |
| 375 | 2x | (4 μM) |
| 376 | nc | (4 μM) |
| 377 | nc | (4 μM) |
| 378 | 16-32 | (4 μM) |
| 379 | 8x | (4 μM) |
| 380 | 8x | (4 μM) |
| 381 | nc | (4 μM) |
| 382 | nc | (4 μM) |
| 383 | 32x (4 μM); | |
| | ≧16x (2μM) | |
| 384 | 8-16x | (4 μM) |
| 385 | 8-16 | (4 μM) |
| 386 | 16x | (4 μM) |
| 387 | 16x | (4 μM) |
| 388 | 16x | (4 μM) |
| 389 | 16x | (4 μM) |
| 390 | 16x | (4 μM) |
| 391 | 16x | (4 μM) |
| 392 | 16x | (4 μM) |
| 393 | 16x | (4 μM) |
| 394 | 8x | (4 μM) |
| 395 | 8x | (4 μM) |
| 396 | 8x | (4 μM) |
| 397 | 8x | (4 μM) |
| 398 | 4x | (4 μM) |
| 399 | 8x | (10 μM) |
| 400 | nc | (4 μM) |
| 401 | nc | (4 μM) |
| 402 | nc | (4 μM) |
| 403 | nc | (4 μM) |
| 404 | nc | (4 μM) |
| 405 | nc | (4 μM) |
| 406 | nc | (4 μM) |
| 407 | nc | (4 μM) |
| 408 | nc | (4 μM) |
| 409 | nc | (4 μM) |
| 410 | nc | (4 μM) |
| 411 | 8x | (4 μM) |
| 412 | nc | (4 μM) |
| 413 | nc | (4 μM) |
| 414 | nc | (4 μM) |
| 415 | nc | (4 μM) |
| 416 | 4x | (10 μM) |
| 417 | 8x | (10 μM) |
| 418 | 16 | (4 μM) |
| 419 | nc | (8 μM) |
| 420 | 16x | (2 μM) |
| 421 | nc | (8 μM) |
| 422 | nc | (8 μM) |
| 423 | nc | (8 μM) |
| 424 | 4x | (2 μM) |
| 425 | nc | (10 μM) |
| 426 | nc | (10 μM) |
| 427 | nc | (8 μM) |
| 428 | 4x | (8 μM) |
| 429 | 4x (1 μM); | |
| | ≧16x (2μM) | |
| 430 | 16x | (2 μM) |
| 431 | 4x (1 μM); | |
| | ≧16x (2 μM) | |
| 432 | 8x | (2 μM) |
| 433 | nc | (8 μM) |
| 434 | 32x | (4 μM) |
| 435 | 16x | (10 μM) |
| 436 | 4x | (2 μM) |
| 437 | nc | (8 μM) |
| 438 | 2x | (4 μM) |
| 439 | nc | (4 μM) |
| 440 | ≧16x | (2 μM) |
| 441 | ≧16x | (2 μM) |

| | |
|---|---|
| 442 | 16x (4 μM); |
| | 8x (2μM) |

*nc = no changeover or no shift

TABLE 21

| | colistin MIC shift, Ab23 | |
|---|---|---|
| No. | at (compd concentration) | |
| 443 | 16x | (4 μM) |
| 444 | 16x | (4 μM) |
| 445 | 16x | (8 μM) |
| 446 | 8-16x | (4 μM) |
| 447 | 4x | (4 μM) |
| 448 | 4x | (4 μM) |
| 449 | 2x | (10 μM) |
| 450 | 2x | (10 μM) |
| 451 | 8x | (10 μM) |
| 452 | 4x | (4 μM) |
| 453 | 8x | (4 μM) |
| 454 | 4x | (4 μM) |
| 455 | 4x | (10 μM) |
| 456 | nc* | (10 μM) |
| 457 | 16x | (4 μM) |
| 458 | 16x | (10 μM) |
| 459 | 8x | (10 μM) |
| 460 | 32x | (4 μM) |
| 461 | 64x | (4 μM) |
| 462 | 64x | (4 μM) |
| 463 | nd** | |
| 464 | nd | |
| 465 | 8-16x | (4 μM) |
| 466 | 8-16x | (4 μM) |
| 467 | 16x | (4 μM) |
| 468 | 16-32x | (4 μM) |
| 469 | 16x | (4 μM) |
| 470 | 2x | (4 μM) |
| 471 | 8x | (4 μM) |
| 472 | 16x | (4 μM) |
| 473 | 16x | (4 μM) |
| 474 | 32x | (4 μM) |
| 475 | 4-8x | (4 μM) |
| 476 | 16x | (4 μM) |
| 477 | 8x | (4 μM) |
| 478 | 4x | (4 μM) |
| 479 | 8x | (4 μM) |
| 480 | 4x | (4 μM) |
| 481 | 8x | (4 μM) |
| 482 | 32x | (4 μM) |
| 483 | 16x | (4 μM) |
| 484 | 16x | (4 μM) |
| 485 | 8x | (10 μM) |
| 486 | 4x | (4 μM) |
| 487 | 32x | (4 μM) |
| 488 | 32x | (4 μM) |
| 489 | 16x | (4 μM) |
| 490 | 16x | (4 μM) |
| 491 | 32x | (4 μM) |
| 492 | 32x | (4 μM) |
| 493 | 16x | (4 μM) |
| 494 | 16x | (4 μM) |
| 495 | 16x | (4 μM) |
| 496 | 16x | (4 μM) |
| 497 | 8-16x | (4 μM) |
| 498 | 8x | (10 μM) |
| 499 | 16x | (8 μM) |
| 500 | 4x | (10 μM) |
| 501 | 8x | (10 μM) |
| 502 | 32x | (4 μM) |
| 503 | 4x | (10 μM) |
| 504 | 16x | (8 μM) |
| 505 | 32x | (4 μM) |
| 506 | 16x | (4 μM) |
| 507 | 8x | (4 μM) |
| 508 | 8x | (4 μM) |
| 509 | 64x | (4 μM) |
| 510 | 16x | (4 μM) |
| 511 | 64x | (4 μM) |
| 512 | 64x | (4 μM) |
| 513 | 32x | (4 μM) |
| 514 | 16x | (4 μM) |
| 515 | 8x | (4 μM) |
| 516 | 16x | (10 μM) |
| 517 | nd | |
| 518 | nd | |
| 519 | nd | |
| 520 | nd | |
| 521 | nd | |
| 522 | nd | |
| 523 | nd | |
| 524 | nd | |
| 525 | 1x | (10 μM) |
| 526 | 1x | (4 μM) |
| 527 | 8x | (10 μM) |
| 528 | 8x | (10 μM) |
| 529 | 8x | (10 μM) |
| 530 | 4x | (10 μM) |
| 531 | 4x | (10 μM) |
| 532 | 4x | (10 μM) |
| 533 | 1x | (10 μM) |
| 534 | 1x | (10 μM) |
| 535 | 1x | (10 μM) |
| 536 | 16x | (10 μM) |
| 537 | 1x | (10 μM) |
| 538 | 1x | (10 μM) |
| 539 | 4x | (10 μM) |
| 540 | 4x | (2 μM) |
| 541 | 32x | (4 μM) |
| 542 | 1x | (10 μM) |
| 543 | 1x | (4 μM) |
| 544 | 8x | (4 μM) |
| 545 | 1x | (10 μM) |
| 546 | 1x | (10 μM) |
| 547 | 1x | (10 μM) |
| 548 | 16x | (10 μM) |
| 549 | 32x | (10 μM) |
| 550 | 64x | (10 μM) |
| 551 | 32x | (10 μM) |
| 552 | 8x | (10 μM) |
| 553 | 1x | (10 μM) |
| 554 | 8x | (10 μM) |
| 555 | 32x | (10 μM) |
| 556 | 4x | (10 μM) |
| 557 | 16x | (10 μM) |
| 558 | 1x | (10 μM) |
| 559 | 8x | (10 μM) |
| 560 | nc | (10 μM) |
| 561 | nc | (10 μM) |
| 562 | 8x | (10 μM) |
| 563 | 8x | (10 μM) |
| 564 | 4x | (4 μM) |
| 565 | 2-4x | (4 μM) |
| 566 | 8x | (4 μM) |
| 567 | 16x | (4 μM) |
| 568 | 4x | (4 μM) |
| 569 | 8x | (4 μM) |
| 570 | 2x | (10 μM) |
| 571 | 4x | (4 μM) |
| 572 | 4x | (4 μM) |
| 573 | 16x | (4 μM) |
| 574 | 8x | (10 μM) |
| 575 | 16x | (4 μM) |
| 576 | 8x | (4 μM) |
| 577 | 16x | (4 μM) |
| 578 | 16x | (4 μM) |
| 579 | 16x | (4 μM) |
| 580 | 8x | (4 μM) |
| 581 | 4x | (4 μM) |
| 582 | 8x | (4 μM) |
| 583 | 4x | (4 μM) |
| 584 | 8x | (4 μM) |
| 585 | 8x | (4 μM) |
| 586 | 16x | (4 μM) |
| 587 | 16x | (10 μM) |
| 588 | nc | (8 μM) |
| 589 | nd | |
| 590 | 8x | (4 μM) |

*nc = no changeover or no shift;
**nd = not determined

TABLE 22

| | colistin MIC shift, Ab23 | |
|---|---|---|
| No. | at (cpd concentration) | |
| 335a | 8x | (4 μM) |
| 362a | 8x | (4 μM) |
| 362b | nc* | (8 μM) |
| 362c | nc | (8 μM) |
| 362d | 32x | (4 μM) |
| 362e | 32x | (4 μM) |
| 362f | 64x | (4 μM) |
| 362g | 16x | (4 μM) |
| 362h | nc | (4 μM) |
| 440a | 4x | (4 μM) |
| 491a | 128x | (4 μM) |
| 524a | 128x | (4 μM) |
| 524b | 16x | (4 μM) |
| 524c | 2x | (4 μM) |
| 524d | 16-32x | (4 μM) |
| 524e | 4x | (4 μM) |
| 524f | 32x | (4 μM) |
| 524g | 16x | (4 μM) |
| 524h | 16x | (4 μM) |
| 524i | 128x | (4 μM) |
| 524j | 32x | (4 μM) |
| 524k | 128x | (4 μM) |
| 524m | 128x | (4 μM) |
| 524n | 32x | (2 μM) |
| 559a | 8x | (8 μM) |
| 559b | 8x | (8 μM) |
| 559c | 2x | (8 μM) |
| 559d | nc | (8 μM) |
| 591 | 32x | (4 μM) |
| 592 | 32x | (4 μM) |

*nc = no changeover or no shift

### Example 3

### Fractional Inhibitory Concentration Index (FICI)

The fractional inhibitory concentration index (FICI) was determined in combination with colistin using the broth microdilution susceptibility test method as described by the CLSI (Methods for Dilution Antimicrobial Susceptibility Tests for Bacteria That Grow Aerobically; Approved Standard-Ninth Edition. 2012. CLSI document M07-A9. CLIS, 940 West Valley Road, Suite 1400, Wayne, Pa. 19087-1898 USA). This checkerboard assay was set up with decreasing concentrations of colistin across the plate from left to right and decreasing concentrations of potentiator from top to bottom. The FICI values were then calculated using the methods of Lorian et al. (Antibiotics in Laboratory Medicine—Fifth Edition. 2005. Lippincott, Williams, & Wilkins, Philadelphia, Pa.). A panel of Gram-negative bacteria with different colistin susceptibilities were tested.

MIC shifts and FICI results for four compounds (shown below) are listed in Table 17. Each of the tested compounds synergized with colistin in the three species of colistin-resistant bacteria tested.

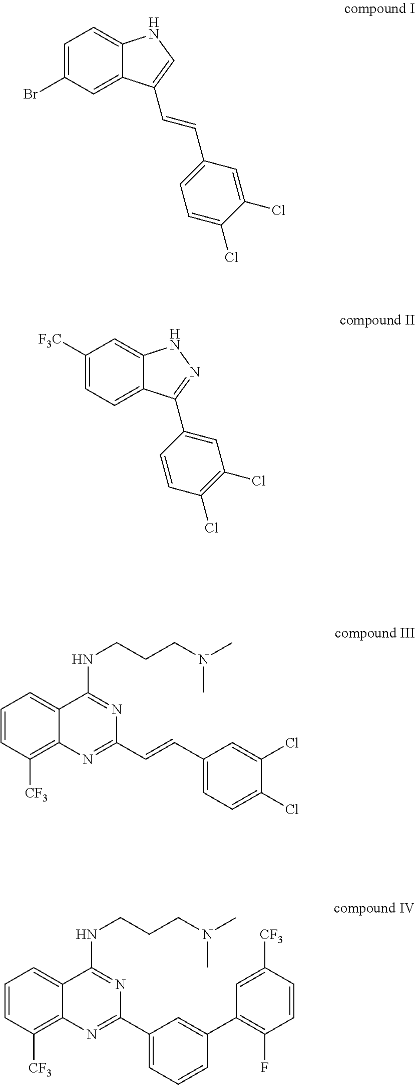 [SMILES: Clc1ccc(/C=C/c2c[nH]c3ccc(Br)cc23)cc1Cl ; FC(F)(F)c1ccc2c(-c3ccc(Cl)c(Cl)c3)n[nH]c2c1 ; CN(C)CCCNc1nc(/C=C/c2ccc(Cl)c(Cl)c2)nc2c(C(F)(F)F)cccc12 ; CN(C)CCCNc1nc(-c2cccc(-c3cc(C(F)(F)F)ccc3F)c2)nc2c(C(F)(F)F)cccc12]

TABLE 17
MIC Shift/FICI Values of Colistin and
Potentiator in Resistant Bacteria*

| colistin- | | | | |
|---|---|---|---|---|
| resistant | compound | compound | compound | compound |
| organism | I | II | III | IV |
| P. aeruginosa | 16x/0.26 | 16x/0.26 | 33x/0.19 | 33x/0.27 |
| K. pneumoniae | 64x/0.30 | 32x/0.39 | 533x/0.17 | 512x/0.21 |
| A. baumannii | 133x/0.25 | 267x/0.15 | 133x/0.21 | 133x/0.24 |

*FICI values of ≦0.5 indicate synergy

### Example 4

### Time-Kill Assay

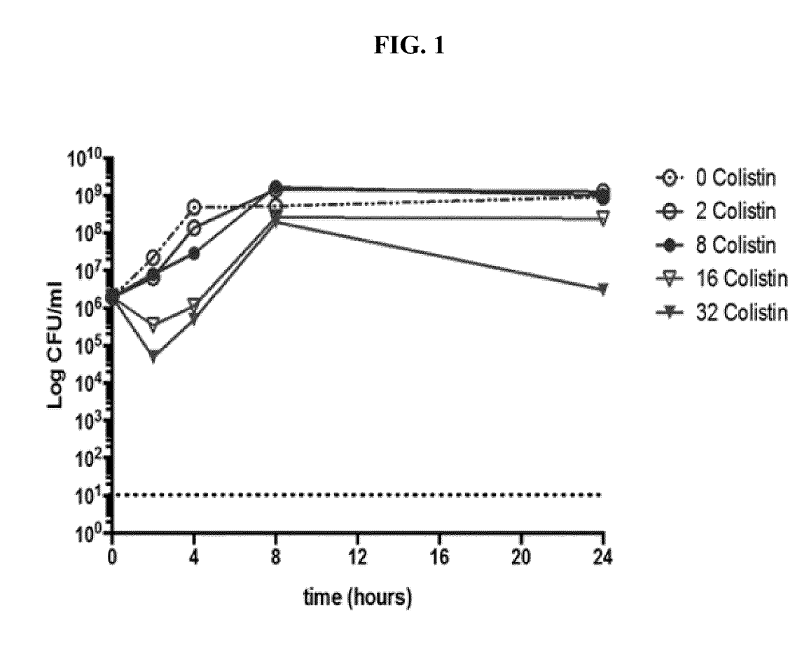

A time-kill assay was used to determine if the addition of potentiator compounds reduced the amount of colistin required to kill Ab23. Colistin and potentiator were diluted to 1000× final concentrations in water and DMSO, respectively. Then, 3 μl of the appropriate dilutions were added to culture tubes. An overnight culture of Ab23 was diluted to 10⁶ CFU/ml, and 3 mL of cells were added to the tubes. The cultures were incubated at 37° C. in an orbital shaker. An aliquot of the starting inoculum was serially diluted in CAMHB and spread on LB agar plates for CFU determination. At 2, 4, 8, and 24 hours, aliquots were removed from each of the culture tubes, diluted, and spread LB agar plates. The plates were incubated overnight at 37° C. The following day, the number of CFUs on each plate was counted, and the number of bacterial in each culture condition was calculated. Exemplary results (for select compounds from Example 3) are shown in FIGS. 1-4. Colistin (“C”) was tested at 2 and 8 g/mL. The select compounds (“P”) were tested at 2 and 10 g/mL. The dotted line indicates the limit of detection.

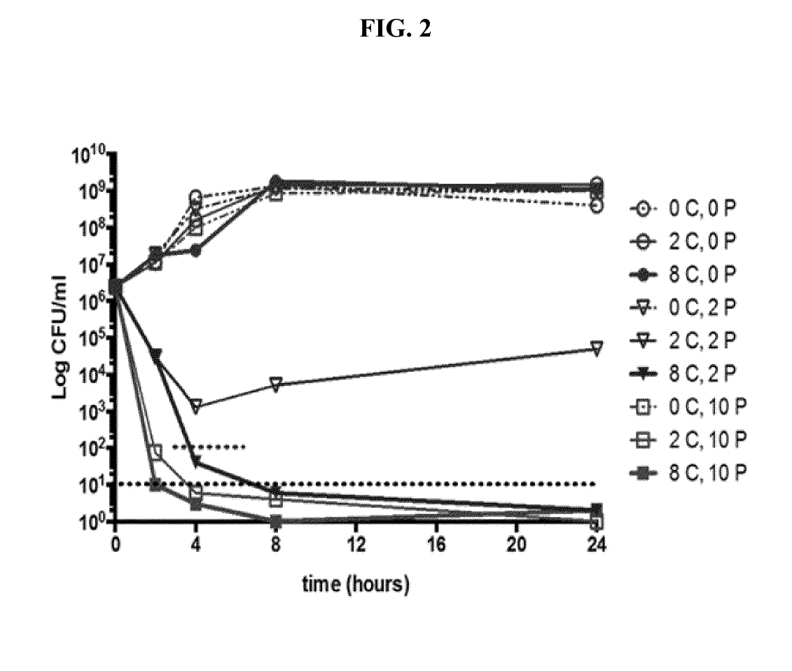

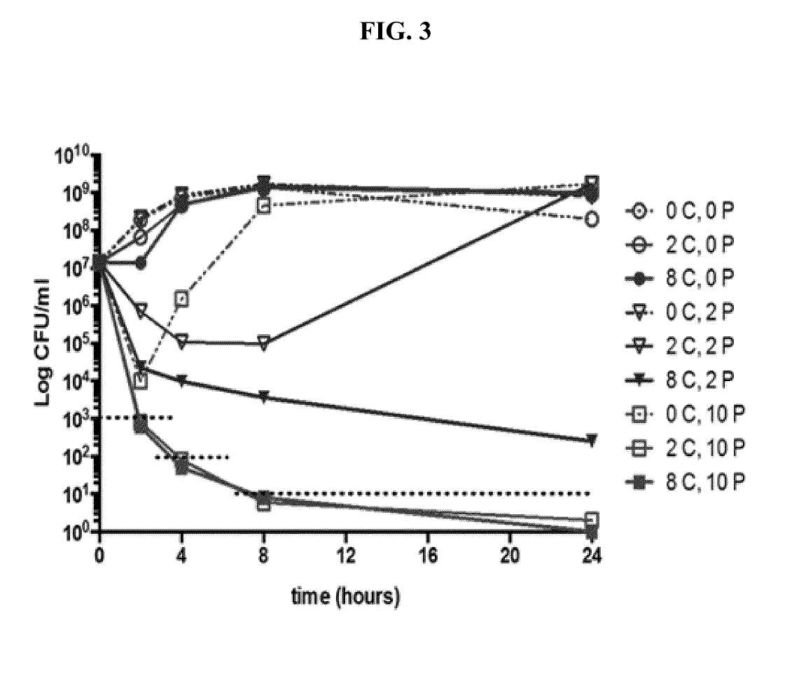

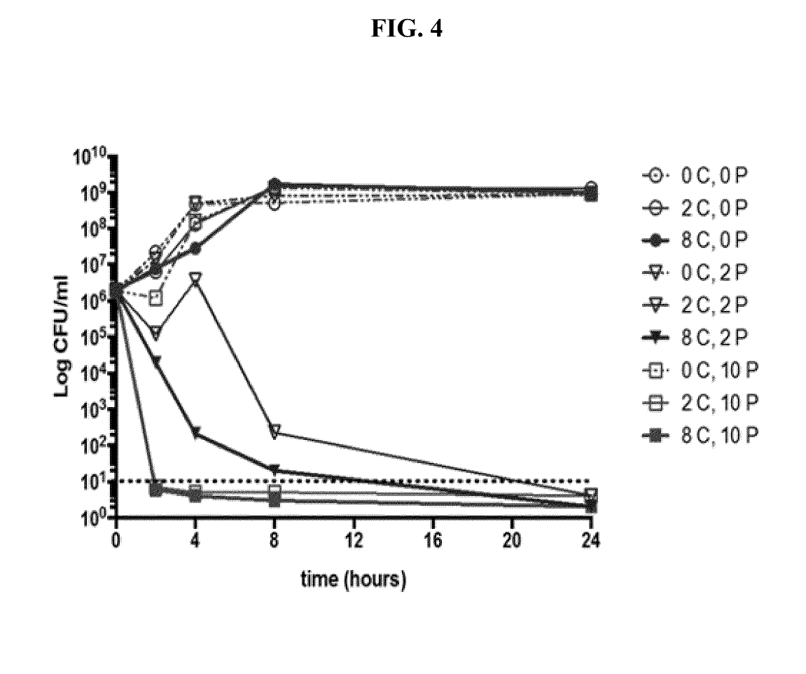

The results of the time-kill assays show that the tested compounds decreased the amount of colistin required to kill Ab23 cells. As shown in FIG. 1, colistin alone was not effective against Ab23. In FIG. 2, only 2 M of compound I was required for effect. As shown in FIG. 3 below, 10 μM of compound III in the absence of colistin reduced Ab23 in two hours, although the effect was not sustained. In contrast, compound I and compound II (FIGS. 2 and 4) had no effect on the cells in the absence of colistin; however, these compounds did potentiate colistin, particularly in the 10 M concentration of potentiator. Overall, in the presence of colistin, compound I, compound II, and compound III were all potent synergists of colistin with compound III demonstrating antimicrobial activity by itself.

### Example 5

### Activity of Compounds of the Invention in Mice

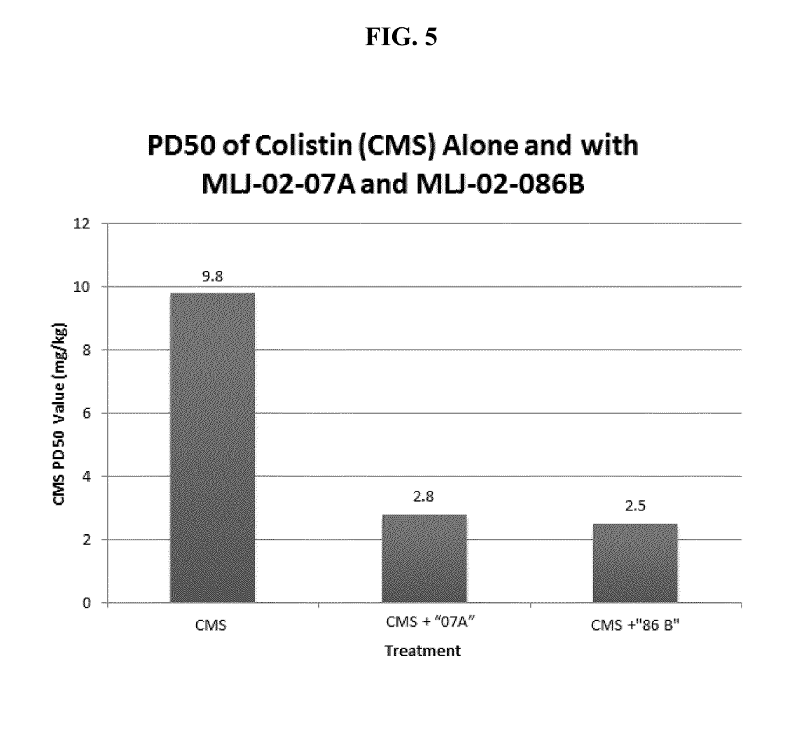

Fully immunocompetent BALB/c mice (n=6/group) were administered approximately 8 log₁₀ CFU of Acinetobacter baumannii clinical isolate Ab23, which induces a bacteremia that is typically fatal within 10 or fewer days. Ab23 is a colistin resistant strain of A baumannii with an MIC value >8-16 fold higher that wild-type A. baumannii. Initial experiments determined the Protective Dose 50% (PD50) against mortality for colistin menthanesulfonate (CMS), a colistin prodrug, administered a time 0, 12, 24, 36, and 48 hr after inoculate administration was 9.8 mg/kg. In subsequent, separate experiments each test compound was administered at 10 mg/kg q12h for 5 doses as above with CMS doses of 2.5, 5, 10, 15 and 20 mg/kg per dose and the new PD50 determined by standard regression analysis. Groups administered CMS alone at 10 mg/kg, and vehicle alone were included as positive and negative controls. As shown in FIG. 5, each test compound reduced the CMS PD50 value by approximately 75%, indicative of a 4-fold greater potency of CMS administered with the test compound compared to CMS alone. The positive and negative control groups replicated previously observed mortality rates.

### Example 6

### Activity of Compounds of the Invention in Humans

One or more appropriate doses of a compound of the invention are administered to a human patient for the treatment of a bacterial infection. Expected results from treatment with the compound, in addition to a reduction or elimination of any signs of the bacterial infection, are reduction and/or elimination of symptoms of infection such as fever, inflammation, and/or pain. The compound of the invention may be selected from Tables 1-15. The bacterial infection may be caused by A. baumannii, Streptococci bacteria, E. coli, or other bacteria.

### Example 7

### Activity of Compounds of the Invention in Humans

One or more appropriate doses of a compound of the invention and an antimicrobial agent are administered to a human patient for the treatment of a bacterial infection. Expected results from treatment with the compound, in addition to a reduction or elimination of any signs of the bacterial infection, are reduction and/or elimination of symptoms of infection such as fever, inflammation, and/or pain. The compound of the invention may be selected from Tables 1-15. The bacterial infection may be caused by A. baumannii, Streptococci bacteria, E. coli, or other bacteria. Examples of the antimicrobial agent include, but are not limited to, a macrocyclic antibiotic, a quinolone antibiotic, a beta-lactam antibiotic, and an aminoglycoside antibiotic. In particular, the antimicrobial agent may be colistin, polymyxin B, meropenem, tobramycin, ciprofloxacin, or levofloxacin.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

What is claimed is:

1. A compound of formula Ia, formula IIa, or formula IIIa: 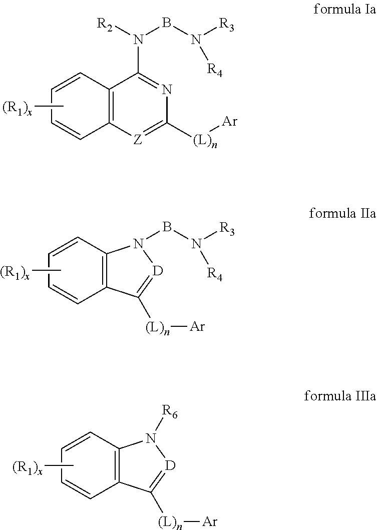
wherein
each L is independently selected from the group consisting of C₁-C₆ alkylene, C₂-C₆ alkenylene, C₂-C₆ alkynylene, —(C₁-C₆ alkylene)-O—, —(C₁-C₆ alkylene)-NR₇—, —NR₇—(C₁-C₆ alkylene)-, —O—(C₁-C₆ alkylene)-, —(C₁-C₆ alkylene)-O—(C₁-C₆ alkylene), —(C₁-C₆ alkylene)-NR₇—(C₁-C₆ alkylene), —NR₇C(O)—, —C(O)—, —C(O)C(O)NR₇—, —C(O)NR₇—, —C(O)NR₇-(alkylene)-, —C(O)—(C₁-C₆ alkylene)-, —(C₁-C₆ alkylene)-C(O)—, and —(C₁-C₆ alkylene)-C(O)—(C₁-C₆ alkylene)-,
n is 0 or 1,
Ar is aryl, optionally substituted with one to five R₅ groups; or heteroaryl, optionally substituted with one to four R₅ groups,
B is an alkylene group, optionally interrupted by an oxygen, a carbonyl (C═O), or sulfonyl group (SO₂), and optionally substituted by one to four groups, which are independently C₁-C₆ alkyl, C₁-C₆ haloalkyl, C₁-C₆ alkoxy, or C₁-C₆ haloalkoxy,
D is CR₇ or N,
each R₁ is independently halogen, C₁-C₆ alkyl, C₁-C₆ haloalkyl, C₁-C₆ alkoxy, C₁-C₆ haloalkoxy, (R₇)₂N—(C₁-C₆ alkylene)-, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl, or optionally substituted heteroaryl; or two R₁ groups are linked together to form an optionally substituted 5- or 6-membered aromatic moiety or an optionally substituted 4- to 7-membered non-aromatic cyclic moiety,
R₂ is hydrogen, optionally substituted C₁-C₆ alkyl, C(O)R₇, or C₁-C₆-alkyloxycarbonyl,
R₃ and R₄ are independently hydrogen, optionally substituted C₁-C₆ alkyl, optionally substituted aryl, C(O)R₇, or C₁-C₆-alkyloxycarbonyl; or R₃ and R₄, together with the nitrogen to which they are attached, form a 4- to 6-membered heterocycle, optionally substituted with one or more groups independently selected from oxo, C₁-C₆ alkyl, C₁-C₆ haloalkyl, C₁-C₆ alkoxy, C₁-C₆ haloalkoxy, SO₂R₈, C(O)OR₇, C(O)N(R₇)₂, C(O)R₇, NR₇SO₂R₈, NR₇C(O)OR₇, NR₇C(O)N(R₇)₂, and NR₇C(O)R₇,
each R₅ is independently halogen, C₁-C₆ alkyl, C₁-C₆ haloalkyl, C₁-C₆ hydroxyalkyl, C₁-C₆ alkoxy, C₁-C₆ haloalkoxy, nitro, cyano, SO₂R₈, SO₂N(R₇)₂, C(O)OR₇, C(O)N(R₇)₂, C(O)R₇, N(R₇)₂, NR₇C(O)R₈, NR₇C(O)N(R₇)₂, NR₇C(O)OR₈, NR₇SO₂R₈, optionally substituted aryl-(C₁-C₆ alkylene), optionally substituted aryl, optionally substituted heterocyclyl-(C₁-C₆ alkylene), optionally substituted heterocyclyl, optionally substituted C₃-C₇ cycloalkyl, or optionally substituted heteroaryl; and/or two R₅ groups are linked together to form an optionally substituted 5- or 6-membered aromatic moiety or an optionally substituted 4- to 7-membered non-aromatic cyclic moiety,
R₆ is hydrogen, optionally substituted C₁-C₆ alkyl, optionally substituted aryl-(C₁-C₆ alkylene), optionally substituted C₁-C₆-alkylcarbonyl, or C₁-C₆-alkyloxycarbonyl,
each R₇ is independently hydrogen, C₁-C₆ alkyl, or C₁-C₆ haloalkyl, and
each R₈ is independently C₁-C₆ alkyl or C₁-C₆ haloalkyl,
Z is CH or N,
x is 0, 1, 2, 3, or 4, and
if x is 0, Ar is not unsubstituted;
or a stereoisomer or a tautomer or a pharmaceutically acceptable salt thereof.

2. The compound of formula Ia as claimed in claim 1 or a stereoisomer or a tautomer or a pharmaceutically acceptable salt thereof.

3. The compound of formula Ia as claimed in claim 1, or a stereoisomer or a tautomer or a pharmaceutically acceptable salt thereof, wherein n is 1.

4. The compound of formula Ia as claimed in claim 1, or a stereoisomer or a tautomer or a pharmaceutically acceptable salt thereof, wherein n is 0.

5. The compound of formula Ia as claimed in claim 1, or a stereoisomer or a tautomer or a pharmaceutically acceptable salt thereof, wherein Z is N.

6. The compound of formula Ia as claimed in claim 1, or a stereoisomer or a tautomer or a pharmaceutically acceptable salt thereof, wherein Z is CH.

7. The compound of formula IIa as claimed in claim 1 or a stereoisomer or a tautomer or a pharmaceutically acceptable salt thereof.

8. The compound of formula IIa as claimed in claim 7, or a stereoisomer or a tautomer or a pharmaceutically acceptable salt thereof, wherein D is CR₇.

9. The compound of formula IIa as claimed in claim 7, or a stereoisomer or a tautomer or a pharmaceutically acceptable salt thereof, wherein D is N.

10. The compound of formula IIIa as claimed in claim 1, or a stereoisomer or a tautomer or a pharmaceutically acceptable salt thereof.

11. The compound of formula IIIa as claimed in claim 10, or a stereoisomer or a tautomer or a pharmaceutically acceptable salt thereof, wherein D is CR₇.

12. The compound of formula IIIa as claimed in claim 10, or a stereoisomer or a tautomer or a pharmaceutically acceptable salt thereof, wherein D is N.

13. The compound of claim 1 selected from the group consisting of the compounds in Tables 1-15.

14. A method of treating a microbial infection in a subject in need thereof, the method comprising administering a compound of claim 1.

15. The method of claim 14, further comprising administering an antimicrobial agent.

16. A method of increasing the activity of an antimicrobial agent, the method comprising administering the antimicrobial agent in combination with at least one compound that potentiates the activity of the antimicrobial agent.

17. The method of claim 16, wherein the at least one compound that potentiates the activity of the antimicrobial agent inhibits the SOS response in bacteria.

18. The method of claim 16, wherein the at least one compound that potentiates the activity of the antimicrobial agent is a compound of claim 1.

19. A composition comprising a compound of claim 1.

20. The composition of claim 19, further comprising an antimicrobial agent.
